(19) United States
(12) Patent Application Publication
(10) Pub. No.: US 2022/0288154 A1
(43) Pub. Date: Sep. 15, 2022

(54) GNRH ANTAGONISTS FOR THE TREATMENT OF ESTROGEN-DEPENDENT DISORDERS

(71) Applicant: ObsEva S.A., Plan-les-Ouates (CH)

(72) Inventors: Jean-Pierre GOTTELAND, Geneva (CH); Elke BESTEL, Geneva (CH)

(21) Appl. No.: 17/633,456

(22) PCT Filed: Aug. 7, 2020
(86) PCT No.: PCT/EP2020/072301

§ 371 Date: Feb. 7, 2022

Related U.S. Application Data
(60) Provisional application No. 63/048,526, filed on Jul. 6, 2020.
(60) Provisional application No. 63/007,632, filed on Apr. 9, 2020.
(60) Provisional application No. 62/914,064, filed on Oct. 11, 2019.
(60) Provisional application No. 62/884,437, filed on Aug. 8, 2019.

(51) Int. Cl. A61K 38/09; A61P 15/02; A61K 31/519; A61P 5/24

(52) U.S. Cl. CPC A61K 38/09; A61P 15/02; A61K 31/519; A61P 5/24

(57) ABSTRACT

The present disclosure provides compositions and methods for treating estrogen-dependent disorders, such as disorders of the female reproductive system, including uterine fibroids and endometriosis, among others. Compounds described herein that may be used to treat such indications include gonadotropin-releasing hormone (GnRH) antagonists. Suitable GnRH antagonists useful in conjunction with the compositions and methods described herein include thieno[3,4d]pyrimidine derivatives, such as 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid and the choline salt thereof, among others. Using the compositions and methods described herein, GnRH antagonists may be periodically administered to a patient, such as one or more times per day, week, or month, over the course of an extended treatment period, such as a treatment period having a duration of multiple years. Advantageously, using the compositions and methods of the present disclosure, the GnRH antagonist may be administered to a patient over a lengthy treatment period without inducing adverse side effects, such as a loss in bone mineral density, which is otherwise known to be a risk associated with GnRH antagonism.

### FIELD OF THE INVENTION

The invention relates to the therapeutic treatment of disorders of the female reproductive system, including uterine fibroids and endometriosis, among others, and reduction of symptoms associated therewith.

### BACKGROUND OF THE INVENTION

Estrogen-dependent disorders represent a challenging class of diseases that have a high incidence in the general population and are often associated with particularly severe symptomology. Uterine fibroids, for example, also referred to as leiomyomata, are among the most common benign tumors in women. Symptoms associated with uterine fibroids commonly include heavy or prolonged menstrual bleeding, pelvic pressure and pelvic organ compression, back pain, and adverse reproductive outcomes. Heavy menstrual bleeding may lead to iron deficiency anemia, a key symptom of uterine fibroids and the leading cause of surgical interventions that may include hysterectomy. Endometriosis is another estrogen-dependent gynecological condition, characterized by the presence of endometrial-like tissue outside the uterus. A chronic inflammatory reaction induced by the ectopic endometrial cells, endometriosis may result in infertility and a variety of pain symptoms including dysmenorrhea, dyspareunia, chronic pelvic pain, dysuria, and dyschezia, among others.

Additional examples of estrogen-dependent diseases include adenomyosis and rectovaginal endometriosis, which are particularly severe endometrial growth disorders characterized by the invasion of endometrial tissue into the uterine myometrium and rectovaginal zones, respectively. The term adenomyosis or uterine adenomyosis is used to describe the presence of both endometrial glands and stroma deep within the myometrium. This condition is associated with hypertrophy and hyperplasia of the subjacent muscle cells, which may ultimately result in an altered size and globulous morphology of the uterus. Due to the severity of this disorder, one of the key symptoms is strong menstrual and even non-menstrual pelvic pain with abnormal uterine bleeding. Like adenomyosis, rectovaginal endometriosis patients present with a variety of pain symptoms including dysmenorrhea, dyspareunia, chronic pelvic pain, dysuria, and dyschezia. Treatment options for rectovaginal endometriosis are limited. Since medical therapies are either ineffective or have considerable side effects, rectovaginal endometriosis patients often undergo surgical procedures to reduce the endometrial node, and may even be subject to resection of the bowel if the node infiltrates the rectal or sigmoidal wall.

There exists a need for new and improved therapies for alleviating the symptoms associated with these and other estrogen-dependent disorders, as well as for treating their underlying pathology.

### SUMMARY OF THE INVENTION

The present disclosure relates to compositions and methods for the treatment of estrogen-dependent disorders, such as uterine fibroids and endometriosis, among others. Using the compositions and methods of the disclosure, a patient, such as a female human patient, may be administered a gonadotropin-releasing hormone (GnRH) antagonist so as to treat the underlying biochemical etiology of one or more of these diseases and/or to alleviate one or more symptoms associated with such conditions. Estrogen-dependent diseases, such as uterine fibroids and endometriosis, among others, emerge due to elevated concentrations of circulating β17-estradiol (E2) in a patient. Particularly, endogenous E2 levels that exceed 60 pg/ml can engender the onset of uterine fibroids, endometriosis, and other estrogen-dependent disorders. Without being limited by mechanism, a patient suffering from an estrogen-dependent disorder may be administered a GnRH antagonist so as to reduce the serum concentration of follicle-stimulating hormone (FSH) and/or luteinizing hormone (LH) in the patient, thereby suppressing ovarian release of E2. The diminished E2 concentration induced by the GnRH antagonist can result in a beneficial effect on symptomology, manifesting in a patient having uterine fibroids, for example, as a reduction in uterine fibroid volume and/or uterine blood loss. Similarly, in the context of a patient having endometriosis, using the compositions and methods described herein, the patient may be administered a GnRH antagonist so as to reduce endogenous E2 levels, thereby diminishing the volume of endometrial tissue extending outside of the uterus and/or alleviating such symptoms as global pelvic pain, dysmenorrhea, dyspareunia, and dyschezia. The compositions and methods of the disclosure can also be used to treat particularly severe endometrial growth disorders, including adenomyosis and rectovaginal endometriosis, among other pathologies mediated by excessive E2 production.

GnRH antagonists that may be used in conjunction with the compositions and methods described herein include optionally substituted thieno[3,4d]pyrimidine derivatives, such as 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid or the choline salt thereof. In some embodiments, the GnRH antagonist is an optionally substituted 3-aminoalkyl pyrimidine-2,4(1H,3H)-dione derivative, such as sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate, also referred to as elagolix, or the carboxylic acid conjugate thereof. The GnRH antagonist may be, for example, an optionally substituted thieno[2,3d]pyrimidine derivative, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, also referred to as relugolix, or a pharmaceutically acceptable salt thereof. In some embodiments, the GnRH antagonist is an optionally substituted propane-1,3-dione derivative, such as (2R)—N-{5-[3-(2,5-difluorophenyl)-2-(1,3-dihydro-2H-benzimidazol-2-ylidene)-3-oxopropanoyl]-2-fluorobenzene-1-sulfonyl}-2-hydroxypropanimidamide, also referred to as opigolix or ASP-1707. Additional GnRH antagonists that may be used in conjunction with the compositions and methods described herein include SKI2670 and BAY-784, as well as derivatives and variants thereof, among others.

Although GnRH antagonist treatment can have the beneficial effect of reducing serum E2 concentration in a patient, and thus, treat the underlying etiology of an estrogen-dependent disease, excessive suppression of endogenous E2 can have harmful side effects. This phenomenon is due, at least in part, to the finding that, although excessive serum E2 levels can promote the development of an estrogen-dependent disease, serum E2 concentration is also positively correlated with bone mineral density (Gallagher, Rheumatic Disease Clinics of North America 27:143-162 (2001)). Accordingly, when serum E2 levels in a patient (e.g., a female human patient) are reduced to certain low concentrations, the patient may experience a reduction in bone mineral density.

In addition to regulating bone mineralization, levels of circulating E2 can influence a patient's concentration of various serum lipids. Particularly, excessive depletion of endogenous E2 has the potential to cause an increase in serum cholesterol and low-density lipoproteins. Thus, patients that are treated for an estrogen-dependent disease (e.g., uterine fibroids, endometriosis, adenomyosis, or rectovaginal endometriosis) have the potential to exhibit elevations in cholesterol and other undesirable serum lipids if their E2 levels are excessively reduced.

The present disclosure is based, in part, on the discovery that GnRH antagonists, and particularly optionally substituted thieno[3,4d]pyrimidine derivatives, such as 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid or a pharmaceutically acceptable salt thereof (e.g., the choline salt thereof), can safely be administered to a patient, such as a human patient suffering from an estrogen-dependent disease, over the course of extended treatment periods, such as those having durations of one year or longer.

Surprisingly, the GnRH antagonist may be administered to the patient over the course of such lengthy treatment periods without inducing significant adverse side effects that are often associated with reduced estrogen levels. During these extended treatment periods, patients not only exhibit a reduction in estrogen-dependent disease symptomology (e.g., a decrease in uterine bleeding among patients having uterine fibroids and a reduction in pelvic pain, dysmenorrhea, dyspareunia, and/or dyschezia in patients having endometriosis, among other symptoms), but also do not experience a substantial reduction in bone mineral density. Additionally, patients treated over extended periods of time with a GnRH antagonist, such as an optionally substituted thieno[3,4d]pyrimidine derivative, (e.g., 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid or a pharmaceutically acceptable salt thereof, such as the choline salt thereof) may exhibit reductions in estrogen-dependent disease symptomology without having their serum cholesterol levels significantly increased. These observations are unexpected, given the involvement of estrogen in bone mineralization and cholesterol regulation, as well as in the onset of estrogen-dependent pathologies.

These surprising treatment outcomes give rise to a series of beneficial dosing regimens. For example, using the compositions and methods describe herein, a patient suffering from an estrogen-dependent disease may be administered a GnRH antagonist (e.g., one or more times per day, week, or month) over the course of extended periods of time, such as over the course of a treatment period lasting one or more years (e.g., a treatment period lasting from one to five years, or more, such as a treatment period lasting 52 weeks, 53 weeks, 54 weeks, 55 weeks, 56 weeks, 57 weeks, 58 weeks, 59 weeks, 60 weeks, 61 weeks, 62 weeks, 63 weeks, 64 weeks, 65 weeks, 66 weeks, 67 weeks, 68 weeks, 69 weeks, 70 weeks, 71 weeks, 72 weeks, 73 weeks, 74 weeks, 75 weeks, 76 weeks, 77 weeks, 78 weeks, 79 weeks, 80 weeks, 81 weeks, 82 weeks, 83 weeks, 84 weeks, 85 weeks, 86 weeks, 87 weeks, 88 weeks, 89 weeks, 90 weeks, 91 weeks, 92 weeks, 93 weeks, 94 weeks, 95 weeks, 96 weeks, 97 weeks, 98 weeks, 99 weeks, 100 weeks, 101 weeks, 102 weeks, 103 weeks, 104 weeks, 105 weeks, 106 weeks, 107 weeks, 108 weeks, 109 weeks, 110 weeks, 111 weeks, 112 weeks, 113 weeks, 114 weeks, 115 weeks, 116 weeks, 117 weeks, 118 weeks, 119 weeks, 120 weeks, 121 weeks, 122 weeks, 123 weeks, 124 weeks, 125 weeks, 126 weeks, 127 weeks, 128 weeks, 129 weeks, 130 weeks, 131 weeks, 132 weeks, 133 weeks, 134 weeks, 135 weeks, 136 weeks, 137 weeks, 138 weeks, 139 weeks, 140 weeks, 141 weeks, 142 weeks, 143 weeks, 144 weeks, 145 weeks, 146 weeks, 147 weeks, 148 weeks, 149 weeks, 150 weeks, or more). During the treatment period, the patient may exhibit a sustained reduction in disease symptomology, a manifestation of the ability of the GnRH antagonist to treat the underlying molecular etiology of the disease by reducing the patient's endogenous estrogen concentration. Advantageously, the GnRH antagonist may alleviate such symptoms without engendering side effects that are associated with hypoestrogenemia, even when the antagonist is periodically administered to the patient over long periods of time.

In a first aspect (“A1”), the disclosure features a method of treating an estrogen-dependent disease in a patient (e.g., a mammalian patient, such as a female human patient) in need thereof. The estrogen-dependent disease may be, for example, uterine fibroids, endometriosis, adenomyosis, or rectovaginal endometriosis. The method may include periodically administering to the patient a therapeutically effective amount of a GnRH antagonist over the course of a treatment period, such as a treatment period having a duration of at least 40 weeks, at least 41 weeks, at least 42 weeks, at least 43 wees, at least 44 weeks, at least 45 weeks, at least 46 weeks, at least 47 weeks, at least 48 weeks, at least 49 weeks, at least 50 weeks, at least 51 weeks, or at least 52 weeks, among other treatment period durations described herein.

In an additional aspect (“A2”), the disclosure features a method of reducing the volume of menstrual blood loss, inducing amenorrhea, reducing the volume of one or more uterine fibroids, preserving hemoglobin, and/or reducing the concentration of follicle-stimulating hormone (FSH), luteinizing hormone (LH), or β17-estradiol (E2) in a human patient diagnosed as having uterine fibroids. The method may include periodically administering to the patient a therapeutically effective amount of a GnRH antagonist over the course of a treatment period, such as a treatment period having a duration of at least 40 weeks, at least 41 weeks, at least 42 weeks, at least 43 wees, at least 44 weeks, at least 45 weeks, at least 46 weeks, at least 47 weeks, at least 48 weeks, at least 49 weeks, at least 50 weeks, at least 51 weeks, or at least 52 weeks, among other treatment period durations described herein.

In an additional aspect (“A3”), the disclosure features a method of reducing the volume of one or more endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having endometriosis. The method may include periodically administering to the patient a therapeutically effective amount of a GnRH antagonist over the course of a treatment period, such as a treatment period having a duration of at least 40 weeks, at least 41 weeks, at least 42 weeks, at least 43 wees, at least 44 weeks, at least 45 weeks, at least 46 weeks, at least 47 weeks, at least 48 weeks, at least 49 weeks, at least 50 weeks, at least 51 weeks, or at least 52 weeks, among other treatment period durations described herein.

In an additional aspect (“A4”), the disclosure features a method of reducing uterine volume, reducing the thickness of the anterior and/or posterior region of the uterine myometrium, reducing pelvic pain, reducing dysmenorrhea, reducing dyspareunia, reducing dyschezia, reducing uterine tenderness, reducing uterine bleeding, inducing amenorrhea, and/or reducing the concentration of FSH, LH, or E2 in a human patient diagnosed as having adenomyosis. The method may include periodically administering to the patient a therapeutically effective amount of a GnRH antagonist over the course of a treatment period, such as a treatment period having a duration of at least 40 weeks, at least 41 weeks, at least 42 weeks, at least 43 wees, at least 44 weeks, at least 45 weeks, at least 46 weeks, at least 47 weeks, at least 48 weeks, at least 49 weeks, at least 50 weeks, at least 51 weeks, or at least 52 weeks, among other treatment period durations described herein.

In an additional aspect (“A5”), the disclosure features a method of reducing the volume of one or more rectovaginal endometriosis nodes, reducing the volume of one or more type III rectovaginal endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having rectovaginal endometriosis. The method may include periodically administering to the patient a therapeutically effective amount of a GnRH antagonist over the course of a treatment period, such as a treatment period having a duration of at least 40 weeks, at least 41 weeks, at least 42 weeks, at least 43 wees, at least 44 weeks, at least 45 weeks, at least 46 weeks, at least 47 weeks, at least 48 weeks, at least 49 weeks, at least 50 weeks, at least 51 weeks, or at least 52 weeks, among other treatment period durations described herein.

In another aspect (“A6”), the disclosure features a method of treating an estrogen-dependent disease (e.g., uterine fibroids, endometriosis, adenomyosis, and/or rectovaginal endometriosis) in a patient (e.g., a female human patient) in need thereof by:
a) periodically administering to the patient a therapeutically effective amount of a GnRH antagonist during a treatment period,
b) monitoring the patient's total cholesterol level, and, if the patient does not exhibit a significant increase in total cholesterol level relative to a measurement of the patient's total cholesterol level obtained prior to the treatment period (i.e., prior to the onset of treatment with the GnRH antagonist),
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit a significant increase in total cholesterol level relative to a measurement of the patient's total cholesterol level obtained prior to the treatment period (i.e., prior to the onset of treatment with the GnRH antagonist), and
c) continuing to periodically administer the GnRH antagonist to the patient for the remainder of the treatment period.

In another aspect (“A7”), the disclosure features a method of treating an estrogen-dependent disease (e.g., uterine fibroids, endometriosis, adenomyosis, and/or rectovaginal endometriosis) in a patient (e.g., a female human patient) in need thereof by:
a) periodically administering to the patient a therapeutically effective amount of a GnRH antagonist during a treatment period,
b) monitoring the patient's low-density lipoprotein-cholesterol level, and, if the patient does not exhibit a significant increase in low-density lipoprotein-cholesterol relative to a measurement of the patient's low-density lipoprotein-cholesterol level obtained prior to the treatment period (i.e., prior to the onset of treatment with the GnRH antagonist),
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit a significant increase in low-density lipoprotein-cholesterol relative to a measurement of the patient's low-density lipoprotein-cholesterol level obtained prior to the treatment period (i.e., prior to the onset of treatment with the GnRH antagonist), and
c) continuing to periodically administer the GnRH antagonist to the patient for the remainder of the treatment period.

In another aspect (“A8”), the disclosure features a method of treating an estrogen-dependent disease (e.g., uterine fibroids, endometriosis, adenomyosis, and/or rectovaginal endometriosis) in a patient (e.g., a female human patient) in need thereof by:
a) periodically administering to the patient a therapeutically effective amount of a GnRH antagonist during a treatment period,
b) monitoring the patient's ratio of low-density lipoprotein-cholesterol to high-density lipoprotein-cholesterol, and, if the patient does not exhibit a significant increase in their ratio of ratio of low-density lipoprotein-cholesterol to high-density lipoprotein-cholesterol relative to a measurement of the patient's ratio of low-density lipoprotein-cholesterol to high-density lipoprotein-cholesterol obtained prior to the treatment period (i.e., prior to the onset of treatment with the GnRH antagonist),
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit a significant increase in their ratio of ratio of low-density lipoprotein-cholesterol to high-density lipoprotein-cholesterol relative to a measurement of the patient's ratio of low-density lipoprotein-cholesterol to high-density lipoprotein-cholesterol obtained prior to the treatment period (i.e., prior to the onset of treatment with the GnRH antagonist), and
c) continuing to periodically administer the GnRH antagonist to the patient for the remainder of the treatment period.

In another aspect (“A9”), the disclosure features a method of treating an estrogen-dependent disease (e.g., uterine fibroids, endometriosis, adenomyosis, and/or rectovaginal endometriosis) in a patient (e.g., a female human patient) in need thereof by:
a) periodically administering to the patient a therapeutically effective amount of a GnRH antagonist during a treatment period,
b) monitoring the patient's low-density lipoprotein-cholesterol level, and, if the patient does not exhibit an increase in low-density lipoprotein-cholesterol level to a level greater than 160 mg/dl,
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit an increase in low-density lipoprotein-cholesterol level to a level greater than 160 mg/dl, and
c) continuing to periodically administer the GnRH antagonist to the patient for the remainder of the treatment period

In another aspect (“A10”), the disclosure features a method of treating an estrogen-dependent disease (e.g., uterine fibroids, endometriosis, adenomyosis, and/or rectovaginal endometriosis) in a patient (e.g., a female human patient) in need thereof by:
a) periodically administering to the patient a therapeutically effective amount of a GnRH antagonist during a treatment period,
b) monitoring the patient's low-density lipoprotein-cholesterol level, and, if the patient does not exhibit an increase in low-density lipoprotein-cholesterol level from a level less than 160 mg/dl (e.g., from a level less than 130 mg/dl) to a level greater than 190 mg/dl,
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit an increase in low-density lipoprotein-cholesterol level from a level less than 160 mg/dl (e.g., from a level less than 130 mg/dl) to a level greater than 190 mg/dl, and
c) continuing to periodically administer the GnRH antagonist to the patient for the remainder of the treatment period.

In another aspect (“A11”), the disclosure features a method of treating an estrogen-dependent disease (e.g., uterine fibroids, endometriosis, adenomyosis, and/or rectovaginal endometriosis) in a patient (e.g., a female human patient) in need thereof by:
a) periodically administering to the patient a therapeutically effective amount of a GnRH antagonist during a treatment period,
b) monitoring the patient's serum triglyceride level, and, if the patient does not exhibit an increase in serum triglyceride level of greater than 30% relative to a measurement of the patient's serum triglyceride level obtained prior to the treatment period (i.e., prior to the onset of treatment with the GnRH antagonist),
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit an increase in serum triglyceride level of greater than 30% relative to a measurement of the patient's serum triglyceride level obtained prior to the treatment period (i.e., prior to the onset of treatment with the GnRH antagonist), and
c) continuing to periodically administer the GnRH antagonist to the patient for the remainder of the treatment period.

In another aspect (“A12”), the disclosure features a method of treating an estrogen-dependent disease (e.g., uterine fibroids, endometriosis, adenomyosis, and/or rectovaginal endometriosis) in a patient (e.g., a female human patient) in need thereof by:
a) periodically administering to the patient a therapeutically effective amount of a GnRH antagonist during a treatment period,
b) monitoring the patient's serum triglyceride level, and, if the patient does not exhibit an increase in serum triglyceride level to a level greater than 200 mg/dl,
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit an increase in serum triglyceride level to a level greater than 200 mg/dl, and
c) continuing to periodically administer the GnRH antagonist to the patient for the remainder of the treatment period.

In another aspect (“A13”), the disclosure features a method of treating an estrogen-dependent disease (e.g., uterine fibroids, endometriosis, adenomyosis, and/or rectovaginal endometriosis) in a patient (e.g., a female human patient) in need thereof by:
a) periodically administering to the patient a therapeutically effective amount of a GnRH antagonist during a treatment period,
b) monitoring the patient's serum triglyceride level, and, if the patient does not exhibit an increase in serum triglyceride level from a level less than 300 mg/dl to a level greater than 500 mg/dl,
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit an increase in serum triglyceride level from a level less than 300 mg/dl to a level greater than 500 mg/dl, and
c) continuing to periodically administer the GnRH antagonist to the patient for the remainder of the treatment period.

In another aspect (“A14”), the disclosure features a method of (i) reducing the volume of menstrual blood loss in a human patient diagnosed as having uterine fibroids, (ii) inducing amenorrhea in a human patient diagnosed as having uterine fibroids, (iii) reducing the volume of one or more uterine fibroids in a human patient diagnosed as having uterine fibroids, (iv) preserving or increasing hemoglobin in a human patient diagnosed as having uterine fibroids, and/or (v) reducing the concentration of FSH, LH, or E2 in a human patient diagnosed as having uterine fibroids by:
a) periodically administering to the patient a therapeutically effective amount of a GnRH antagonist during a treatment period,
b) monitoring the patient's total cholesterol level, and, if the patient does not exhibit a significant increase in total cholesterol level relative to a measurement of the patient's total cholesterol level obtained prior to the treatment period (i.e., prior to the onset of treatment with the GnRH antagonist),
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit a significant increase in total cholesterol level relative to a measurement of the patient's total cholesterol level obtained prior to the treatment period (i.e., prior to the onset of treatment with the GnRH antagonist), and
c) continuing to periodically administer the GnRH antagonist to the patient for the remainder of the treatment period.

In another aspect (“A15”), the disclosure features a method of (i) reducing the volume of menstrual blood loss in a human patient diagnosed as having uterine fibroids, (ii) inducing amenorrhea in a human patient diagnosed as having uterine fibroids, (iii) reducing the volume of one or more uterine fibroids in a human patient diagnosed as having uterine fibroids, (iv) preserving or increasing hemoglobin in a human patient diagnosed as having uterine fibroids, and/or (v) reducing the concentration of FSH, LH, or E2 in a human patient diagnosed as having uterine fibroids by:
a) periodically administering to the patient a therapeutically effective amount of a GnRH antagonist during a treatment period,
b) monitoring the patient's low-density lipoprotein-cholesterol level, and, if the patient does not exhibit a significant increase in low-density lipoprotein-cholesterol relative to a measurement of the patient's low-density lipoprotein-cholesterol level obtained prior to the treatment period (i.e., prior to the onset of treatment with the GnRH antagonist),
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit a significant increase in low-density lipoprotein-cholesterol relative to a measurement of the patient's low-density lipoprotein-cholesterol level obtained prior to the treatment period (i.e., prior to the onset of treatment with the GnRH antagonist), and
c) continuing to periodically administer the GnRH antagonist to the patient for the remainder of the treatment period.

In another aspect (“A16”), the disclosure features a method of (i) reducing the volume of menstrual blood loss in a human patient diagnosed as having uterine fibroids, (ii) inducing amenorrhea in a human patient diagnosed as having uterine fibroids, (iii) reducing the volume of one or more uterine fibroids in a human patient diagnosed as having uterine fibroids, (iv) preserving or increasing hemoglobin in a human patient diagnosed as having uterine fibroids, and/or (v) reducing the concentration of FSH, LH, or E2 in a human patient diagnosed as having uterine fibroids by:
a) periodically administering to the patient a therapeutically effective amount of a GnRH antagonist during a treatment period,
b) monitoring the patient's ratio of low-density lipoprotein-cholesterol to high-density lipoprotein-cholesterol, and, if the patient does not exhibit a significant increase in their ratio of ratio of low-density lipoprotein-cholesterol to high-density lipoprotein-cholesterol relative to a measurement of the patient's ratio of low-density lipoprotein-cholesterol to high-density lipoprotein-cholesterol obtained prior to the treatment period (i.e., prior to the onset of treatment with the GnRH antagonist),
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit a significant increase in their ratio of ratio of low-density lipoprotein-cholesterol to high-density lipoprotein-cholesterol relative to a measurement of the patient's ratio of low-density lipoprotein-cholesterol to high-density lipoprotein-cholesterol obtained prior to the treatment period (i.e., prior to the onset of treatment with the GnRH antagonist), and
c) continuing to periodically administer the GnRH antagonist to the patient for the remainder of the treatment period.

In another aspect (“A17”), the disclosure features a method of (i) reducing the volume of menstrual blood loss in a human patient diagnosed as having uterine fibroids, (ii) inducing amenorrhea in a human patient diagnosed as having uterine fibroids, (iii) reducing the volume of one or more uterine fibroids in a human patient diagnosed as having uterine fibroids, (iv) preserving or increasing hemoglobin in a human patient diagnosed as having uterine fibroids, and/or (v) reducing the concentration of FSH, LH, or E2 in a human patient diagnosed as having uterine fibroids by:
a) periodically administering to the patient a therapeutically effective amount of a GnRH antagonist during a treatment period,
b) monitoring the patient's low-density lipoprotein-cholesterol level, and, if the patient does not exhibit an increase in low-density lipoprotein-cholesterol level to a level greater than 160 mg/dl,
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit an increase in low-density lipoprotein-cholesterol level to a level greater than 160 mg/dl, and
c) continuing to periodically administer the GnRH antagonist to the patient for the remainder of the treatment period

In another aspect (“A18”), the disclosure features a method of (i) reducing the volume of menstrual blood loss in a human patient diagnosed as having uterine fibroids, (ii) inducing amenorrhea in a human patient diagnosed as having uterine fibroids, (iii) reducing the volume of one or more uterine fibroids in a human patient diagnosed as having uterine fibroids, (iv) preserving or increasing hemoglobin in a human patient diagnosed as having uterine fibroids, and/or (v) reducing the concentration of FSH, LH, or E2 in a human patient diagnosed as having uterine fibroids by:
a) periodically administering to the patient a therapeutically effective amount of a GnRH antagonist during a treatment period,
b) monitoring the patient's low-density lipoprotein-cholesterol level, and, if the patient does not exhibit an increase in low-density lipoprotein-cholesterol level from a level less than 160 mg/dl (e.g., from a level less than 130 mg/dl) to a level greater than 190 mg/dl,
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit an increase in low-density lipoprotein-cholesterol level from a level less than 160 mg/dl (e.g., from a level less than 130 mg/dl) to a level greater than 190 mg/dl, and
c) continuing to periodically administer the GnRH antagonist to the patient for the remainder of the treatment period.

In another aspect (“A19”), the disclosure features a method of (i) reducing the volume of menstrual blood loss in a human patient diagnosed as having uterine fibroids, (ii) inducing amenorrhea in a human patient diagnosed as having uterine fibroids, (iii) reducing the volume of one or more uterine fibroids in a human patient diagnosed as having uterine fibroids, (iv) preserving or increasing hemoglobin in a human patient diagnosed as having uterine fibroids, and/or (v) reducing the concentration of FSH, LH, or E2 in a human patient diagnosed as having uterine fibroids by:
a) periodically administering to the patient a therapeutically effective amount of a GnRH antagonist during a treatment period,
b) monitoring the patient's serum triglyceride level, and, if the patient does not exhibit an increase in serum triglyceride level of greater than 30% relative to a measurement of the patient's serum triglyceride level obtained prior to the treatment period (i.e., prior to the onset of treatment with the GnRH antagonist),
c) continuing to periodically administer the GnRH antagonist to the patient for the remainder of the treatment period.
   For example, in some embodiments of this aspect, the method includes:
a) periodically administering to the patient a therapeutically effective amount of a GnRH antagonist during a treatment period,
b) determining that the patient does not exhibit an increase in serum triglyceride level of greater than 30% relative to a measurement of the patient's serum triglyceride level obtained prior to the treatment period (i.e., prior to the onset of treatment with the GnRH antagonist), and
c) continuing to periodically administer the GnRH antagonist to the patient for the remainder of the treatment period.

In another aspect (“A20”), the disclosure features a method of (i) reducing the volume of menstrual blood loss in a human patient diagnosed as having uterine fibroids, (ii) inducing amenorrhea in a human patient diagnosed as having uterine fibroids, (iii) reducing the volume of one or more uterine fibroids in a human patient diagnosed as having uterine fibroids, (iv) preserving or increasing hemoglobin in a human patient diagnosed as having uterine fibroids, and/or (v) reducing the concentration of FSH, LH, or E2 in a human patient diagnosed as having uterine fibroids by:
a) periodically administering to the patient a therapeutically effective amount of a GnRH antagonist during a treatment period,
b) monitoring the patient's serum triglyceride level, and, if the patient does not exhibit an increase in serum triglyceride level to a level greater than 200 mg/dl,
c) continuing to periodically administer the GnRH antagonist to the patient for the remainder of the treatment period.
   For example, in some embodiments of this aspect, the method includes:
a) periodically administering to the patient a therapeutically effective amount of a GnRH antagonist during a treatment period,
b) determining that the patient does not exhibit an increase in serum triglyceride level to a level greater than 200 mg/dl, and
c) continuing to periodically administer the GnRH antagonist to the patient for the remainder of the treatment period.

In another aspect (“A21”), the disclosure features a method of (i) reducing the volume of menstrual blood loss in a human patient diagnosed as having uterine fibroids, (ii) inducing amenorrhea in a human patient diagnosed as having uterine fibroids, (iii) reducing the volume of one or more uterine fibroids in a human patient diagnosed as having uterine fibroids, (iv) preserving or increasing hemoglobin in a human patient diagnosed as having uterine fibroids, and/or (v) reducing the concentration of FSH, LH, or E2 in a human patient diagnosed as having uterine fibroids by:
a) periodically administering to the patient a therapeutically effective amount of a GnRH antagonist during a treatment period,
b) monitoring the patient's serum triglyceride level, and, if the patient does not exhibit an increase in serum triglyceride level from a level less than 300 mg/dl to a level greater than 500 mg/dl,
c) continuing to periodically administer the GnRH antagonist to the patient for the remainder of the treatment period.
   For example, in some embodiments of this aspect, the method includes:
a) periodically administering to the patient a therapeutically effective amount of a GnRH antagonist during a treatment period,
b) determining that the patient does not exhibit an increase in serum triglyceride level from a level less than 300 mg/dl to a level greater than 500 mg/dl, and
c) continuing to periodically administer the GnRH antagonist to the patient for the remainder of the treatment period.

In another aspect (“A22”), the disclosure features a method of (i) reducing the volume of one or more endometriosis nodes in a human patient diagnosed as having endometriosis, (ii) reducing pelvic pain in a human patient diagnosed as having endometriosis, (iii) reducing dysmenorrhea in a human patient diagnosed as having endometriosis, (iv) reducing dyspareunia in a human patient diagnosed as having endometriosis, (v) reducing dyschezia in a human patient diagnosed as having endometriosis, (vi) reducing uterine bleeding in a human patient diagnosed as having endometriosis, (vii) inducing amenorrhea in a human patient diagnosed as having endometriosis, and/or (viii) reducing the concentration of FSH, LH, or E2 in a human patient diagnosed as having endometriosis by:
a) periodically administering to the patient a therapeutically effective amount of a GnRH antagonist during a treatment period,
b) monitoring the patient's total cholesterol level, and, if the patient does not exhibit a significant increase in total cholesterol level relative to a measurement of the patient's total cholesterol level obtained prior to the treatment period (i.e., prior to the onset of treatment with the GnRH antagonist),
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit a significant increase in total cholesterol level relative to a measurement of the patient's total cholesterol level obtained prior to the treatment period (i.e., prior to the onset of treatment with the GnRH antagonist), and
c) continuing to periodically administer the GnRH antagonist to the patient for the remainder of the treatment period.

In another aspect (“A23”), the disclosure features a method of (i) reducing the volume of one or more endometriosis nodes in a human patient diagnosed as having endometriosis, (ii) reducing pelvic pain in a human patient diagnosed as having endometriosis, (iii) reducing dysmenorrhea in a human patient diagnosed as having endometriosis, (iv) reducing dyspareunia in a human patient diagnosed as having endometriosis, (v) reducing dyschezia in a human patient diagnosed as having endometriosis, (vi) reducing uterine bleeding in a human patient diagnosed as having endometriosis, (vii) inducing amenorrhea in a human patient diagnosed as having endometriosis, and/or (viii) reducing the concentration of FSH, LH, or E2 in a human patient diagnosed as having endometriosis by:
a) periodically administering to the patient a therapeutically effective amount of a GnRH antagonist during a treatment period,
b) monitoring the patient's low-density lipoprotein-cholesterol level, and, if the patient does not exhibit a significant increase in low-density lipoprotein-cholesterol relative to a measurement of the patient's low-density lipoprotein-cholesterol level obtained prior to the treatment period (i.e., prior to the onset of treatment with the GnRH antagonist),
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit a significant increase in low-density lipoprotein-cholesterol relative to a measurement of the patient's low-density lipoprotein-cholesterol level obtained prior to the treatment period (i.e., prior to the onset of treatment with the GnRH antagonist), and
c) continuing to periodically administer the GnRH antagonist to the patient for the remainder of the treatment period.

In another aspect (“A24”), the disclosure features a method of (i) reducing the volume of one or more endometriosis nodes in a human patient diagnosed as having endometriosis, (ii) reducing pelvic pain in a human patient diagnosed as having endometriosis, (iii) reducing dysmenorrhea in a human patient diagnosed as having endometriosis, (iv) reducing dyspareunia in a human patient diagnosed as having endometriosis, (v) reducing dyschezia in a human patient diagnosed as having endometriosis, (vi) reducing uterine bleeding in a human patient diagnosed as having endometriosis, (vii) inducing amenorrhea in a human patient diagnosed as having endometriosis, and/or (viii) reducing the concentration of FSH, LH, or E2 in a human patient diagnosed as having endometriosis by:
a) periodically administering to the patient a therapeutically effective amount of a GnRH antagonist during a treatment period,
b) monitoring the patient's ratio of low-density lipoprotein-cholesterol to high-density lipoprotein-cholesterol, and, if the patient does not exhibit a significant increase in their ratio of ratio of low-density lipoprotein-cholesterol to high-density lipoprotein-cholesterol relative to a measurement of the patient's ratio of low-density lipoprotein-cholesterol to high-density lipoprotein-cholesterol obtained prior to the treatment period (i.e., prior to the onset of treatment with the GnRH antagonist),
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit a significant increase in their ratio of ratio of low-density lipoprotein-cholesterol to high-density lipoprotein-cholesterol relative to a measurement of the patient's ratio of low-density lipoprotein-cholesterol to high-density lipoprotein-cholesterol obtained prior to the treatment period (i.e., prior to the onset of treatment with the GnRH antagonist), and
c) continuing to periodically administer the GnRH antagonist to the patient for the remainder of the treatment period.

In another aspect (“A25”), the disclosure features a method of (i) reducing the volume of one or more endometriosis nodes in a human patient diagnosed as having endometriosis, (ii) reducing pelvic pain in a human patient diagnosed as having endometriosis, (iii) reducing dysmenorrhea in a human patient diagnosed as having endometriosis, (iv) reducing dyspareunia in a human patient diagnosed as having endometriosis, (v) reducing dyschezia in a human patient diagnosed as having endometriosis, (vi) reducing uterine bleeding in a human patient diagnosed as having endometriosis, (vii) inducing amenorrhea in a human patient diagnosed as having endometriosis, and/or (viii) reducing the concentration of FSH, LH, or E2 in a human patient diagnosed as having endometriosis by:
a) periodically administering to the patient a therapeutically effective amount of a GnRH antagonist during a treatment period,
b) monitoring the patient's low-density lipoprotein-cholesterol level, and, if the patient does not exhibit an increase in low-density lipoprotein-cholesterol level to a level greater than 160 mg/dl,
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit an increase in low-density lipoprotein-cholesterol level to a level greater than 160 mg/dl, and
c) continuing to periodically administer the GnRH antagonist to the patient for the remainder of the treatment period

In another aspect (“A26”), the disclosure features a method of (i) reducing the volume of one or more endometriosis nodes in a human patient diagnosed as having endometriosis, (ii) reducing pelvic pain in a human patient diagnosed as having endometriosis, (iii) reducing dysmenorrhea in a human patient diagnosed as having endometriosis, (iv) reducing dyspareunia in a human patient diagnosed as having endometriosis, (v) reducing dyschezia in a human patient diagnosed as having endometriosis, (vi) reducing uterine bleeding in a human patient diagnosed as having endometriosis, (vii) inducing amenorrhea in a human patient diagnosed as having endometriosis, and/or (viii) reducing the concentration of FSH, LH, or E2 in a human patient diagnosed as having endometriosis by:
a) periodically administering to the patient a therapeutically effective amount of a GnRH antagonist during a treatment period,
b) monitoring the patient's low-density lipoprotein-cholesterol level, and, if the patient does not exhibit an increase in low-density lipoprotein-cholesterol level from a level less than 160 mg/dl (e.g., from a level less than 130 mg/dl) to a level greater than 190 mg/dl,
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit an increase in low-density lipoprotein-cholesterol level from a level less than 160 mg/dl (e.g., from a level less than 130 mg/dl) to a level greater than 190 mg/dl, and
c) continuing to periodically administer the GnRH antagonist to the patient for the remainder of the treatment period.

In another aspect (“A27”), the disclosure features a method of (i) reducing the volume of one or more endometriosis nodes in a human patient diagnosed as having endometriosis, (ii) reducing pelvic pain in a human patient diagnosed as having endometriosis, (iii) reducing dysmenorrhea in a human patient diagnosed as having endometriosis, (iv) reducing dyspareunia in a human patient diagnosed as having endometriosis, (v) reducing dyschezia in a human patient diagnosed as having endometriosis, (vi) reducing uterine bleeding in a human patient diagnosed as having endometriosis, (vii) inducing amenorrhea in a human patient diagnosed as having endometriosis, and/or (viii) reducing the concentration of FSH, LH, or E2 in a human patient diagnosed as having endometriosis by:
a) periodically administering to the patient a therapeutically effective amount of a GnRH antagonist during a treatment period,
b) monitoring the patient's serum triglyceride level, and, if the patient does not exhibit an increase in serum triglyceride level of greater than 30% relative to a measurement of the patient's serum triglyceride level obtained prior to the treatment period (i.e., prior to the onset of treatment with the GnRH antagonist),
c) continuing to periodically administer the GnRH antagonist to the patient for the remainder of the treatment period.
   For example, in some embodiments of this aspect, the method includes:
a) periodically administering to the patient a therapeutically effective amount of a GnRH antagonist during a treatment period,
b) determining that the patient does not exhibit an increase in serum triglyceride level of greater than 30% relative to a measurement of the patient's serum triglyceride level obtained prior to the treatment period (i.e., prior to the onset of treatment with the GnRH antagonist), and
c) continuing to periodically administer the GnRH antagonist to the patient for the remainder of the treatment period.

In another aspect (“A28”), the disclosure features a method of (i) reducing the volume of one or more endometriosis nodes in a human patient diagnosed as having endometriosis, (ii) reducing pelvic pain in a human patient diagnosed as having endometriosis, (iii) reducing dysmenorrhea in a human patient diagnosed as having endometriosis, (iv) reducing dyspareunia in a human patient diagnosed as having endometriosis, (v) reducing dyschezia in a human patient diagnosed as having endometriosis, (vi) reducing uterine bleeding in a human patient diagnosed as having endometriosis, (vii) inducing amenorrhea in a human patient diagnosed as having endometriosis, and/or (viii) reducing the concentration of FSH, LH, or E2 in a human patient diagnosed as having endometriosis by:
a) periodically administering to the patient a therapeutically effective amount of a GnRH antagonist during a treatment period,
b) monitoring the patient's serum triglyceride level, and, if the patient does not exhibit an increase in serum triglyceride level to a level greater than 200 mg/dl,
c) continuing to periodically administer the GnRH antagonist to the patient for the remainder of the treatment period.
   For example, in some embodiments of this aspect, the method includes:
a) periodically administering to the patient a therapeutically effective amount of a GnRH antagonist during a treatment period,
b) determining that the patient does not exhibit an increase in serum triglyceride level to a level greater than 200 mg/dl, and
c) continuing to periodically administer the GnRH antagonist to the patient for the remainder of the treatment period.

In another aspect (“A29”), the disclosure features a method of (i) reducing the volume of one or more endometriosis nodes in a human patient diagnosed as having endometriosis, (ii) reducing pelvic pain in a human patient diagnosed as having endometriosis, (iii) reducing dysmenorrhea in a human patient diagnosed as having endometriosis, (iv) reducing dyspareunia in a human patient diagnosed as having endometriosis, (v) reducing dyschezia in a human patient diagnosed as having endometriosis, (vi) reducing uterine bleeding in a human patient diagnosed as having endometriosis, (vii) inducing amenorrhea in a human patient diagnosed as having endometriosis, and/or (viii) reducing the concentration of FSH, LH, or E2 in a human patient diagnosed as having endometriosis by:
a) periodically administering to the patient a therapeutically effective amount of a GnRH antagonist during a treatment period,
b) monitoring the patient's serum triglyceride level, and, if the patient does not exhibit an increase in serum triglyceride level from a level less than 300 mg/dl to a level greater than 500 mg/dl,
c) continuing to periodically administer the GnRH antagonist to the patient for the remainder of the treatment period.
   For example, in some embodiments of this aspect, the method includes:
a) periodically administering to the patient a therapeutically effective amount of a GnRH antagonist during a treatment period,
b) determining that the patient does not exhibit an increase in serum triglyceride level from a level less than 300 mg/dl to a level greater than 500 mg/dl, and
c) continuing to periodically administer the GnRH antagonist to the patient for the remainder of the treatment period.

In another aspect (“A30”), the disclosure features a method of (i) reducing the volume of one or more rectovaginal endometriosis nodes in a human patient diagnosed as having rectovaginal endometriosis, (ii) reducing the volume of one or more type III rectovaginal endometriosis nodes in a human patient diagnosed as having rectovaginal endometriosis, (iii) reducing pelvic pain in a human patient diagnosed as having rectovaginal endometriosis, (iv) reducing dysmenorrhea in a human patient diagnosed as having rectovaginal endometriosis, (v) reducing dyspareunia in a human patient diagnosed as having rectovaginal endometriosis, (vi) reducing dyschezia in a human patient diagnosed as having rectovaginal endometriosis, (vii) reducing uterine bleeding in a human patient diagnosed as having rectovaginal endometriosis, (viii) inducing amenorrhea in a human patient diagnosed as having rectovaginal endometriosis, and/or (ix) and/or (viii) reducing the concentration of FSH, LH, or E2 in a human patient diagnosed as having rectovaginal endometriosis by:
a) periodically administering to the patient a therapeutically effective amount of a GnRH antagonist during a treatment period,
b) monitoring the patient's total cholesterol level, and, if the patient does not exhibit a significant increase in total cholesterol level relative to a measurement of the patient's total cholesterol level obtained prior to the treatment period (i.e., prior to the onset of treatment with the GnRH antagonist),
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit a significant increase in total cholesterol level relative to a measurement of the patient's total cholesterol level obtained prior to the treatment period (i.e., prior to the onset of treatment with the GnRH antagonist), and
c) continuing to periodically administer the GnRH antagonist to the patient for the remainder of the treatment period.

In another aspect (“A31”), the disclosure features a method of (i) reducing the volume of one or more rectovaginal endometriosis nodes in a human patient diagnosed as having rectovaginal endometriosis, (ii) reducing the volume of one or more type III rectovaginal endometriosis nodes in a human patient diagnosed as having rectovaginal endometriosis, (iii) reducing pelvic pain in a human patient diagnosed as having rectovaginal endometriosis, (iv) reducing dysmenorrhea in a human patient diagnosed as having rectovaginal endometriosis, (v) reducing dyspareunia in a human patient diagnosed as having rectovaginal endometriosis, (vi) reducing dyschezia in a human patient diagnosed as having rectovaginal endometriosis, (vii) reducing uterine bleeding in a human patient diagnosed as having rectovaginal endometriosis, (viii) inducing amenorrhea in a human patient diagnosed as having rectovaginal endometriosis, and/or (ix) and/or (viii) reducing the concentration of FSH, LH, or E2 in a human patient diagnosed as having rectovaginal endometriosis by:
a) periodically administering to the patient a therapeutically effective amount of a GnRH antagonist during a treatment period,
b) monitoring the patient's low-density lipoprotein-cholesterol level, and, if the patient does not exhibit a significant increase in low-density lipoprotein-cholesterol relative to a measurement of the patient's low-density lipoprotein-cholesterol level obtained prior to the treatment period (i.e., prior to the onset of treatment with the GnRH antagonist),
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit a significant increase in low-density lipoprotein-cholesterol relative to a measurement of the patient's low-density lipoprotein-cholesterol level obtained prior to the treatment period (i.e., prior to the onset of treatment with the GnRH antagonist), and
c) continuing to periodically administer the GnRH antagonist to the patient for the remainder of the treatment period.

In another aspect (“A32”), the disclosure features a method of (i) reducing the volume of one or more rectovaginal endometriosis nodes in a human patient diagnosed as having rectovaginal endometriosis, (ii) reducing the volume of one or more type III rectovaginal endometriosis nodes in a human patient diagnosed as having rectovaginal endometriosis, (iii) reducing pelvic pain in a human patient diagnosed as having rectovaginal endometriosis, (iv) reducing dysmenorrhea in a human patient diagnosed as having rectovaginal endometriosis, (v) reducing dyspareunia in a human patient diagnosed as having rectovaginal endometriosis, (vi) reducing dyschezia in a human patient diagnosed as having rectovaginal endometriosis, (vii) reducing uterine bleeding in a human patient diagnosed as having rectovaginal endometriosis, (viii) inducing amenorrhea in a human patient diagnosed as having rectovaginal endometriosis, and/or (ix) and/or (viii) reducing the concentration of FSH, LH, or E2 in a human patient diagnosed as having rectovaginal endometriosis by:
a) periodically administering to the patient a therapeutically effective amount of a GnRH antagonist during a treatment period,
b) monitoring the patient's ratio of low-density lipoprotein-cholesterol to high-density lipoprotein-cholesterol, and, if the patient does not exhibit a significant increase in their ratio of ratio of low-density lipoprotein-cholesterol to high-density lipoprotein-cholesterol relative to a measurement of the patient's ratio of low-density lipoprotein-cholesterol to high-density lipoprotein-cholesterol obtained prior to the treatment period (i.e., prior to the onset of treatment with the GnRH antagonist),
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit a significant increase in their ratio of ratio of low-density lipoprotein-cholesterol to high-density lipoprotein-cholesterol relative to a measurement of the patient's ratio of low-density lipoprotein-cholesterol to high-density lipoprotein-cholesterol obtained prior to the treatment period (i.e., prior to the onset of treatment with the GnRH antagonist), and
c) continuing to periodically administer the GnRH antagonist to the patient for the remainder of the treatment period.

In another aspect (“A33”), the disclosure features a method of (i) reducing the volume of one or more rectovaginal endometriosis nodes in a human patient diagnosed as having rectovaginal endometriosis, (ii) reducing the volume of one or more type III rectovaginal endometriosis nodes in a human patient diagnosed as having rectovaginal endometriosis, (iii) reducing pelvic pain in a human patient diagnosed as having rectovaginal endometriosis, (iv) reducing dysmenorrhea in a human patient diagnosed as having rectovaginal endometriosis, (v) reducing dyspareunia in a human patient diagnosed as having rectovaginal endometriosis, (vi) reducing dyschezia in a human patient diagnosed as having rectovaginal endometriosis, (vii) reducing uterine bleeding in a human patient diagnosed as having rectovaginal endometriosis, (viii) inducing amenorrhea in a human patient diagnosed as having rectovaginal endometriosis, and/or (ix) and/or (viii) reducing the concentration of FSH, LH, or E2 in a human patient diagnosed as having rectovaginal endometriosis by:
a) periodically administering to the patient a therapeutically effective amount of a GnRH antagonist during a treatment period,
b) monitoring the patient's low-density lipoprotein-cholesterol level, and, if the patient does not exhibit an increase in low-density lipoprotein-cholesterol level to a level greater than 160 mg/dl,
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit an increase in low-density lipoprotein-cholesterol level to a level greater than 160 mg/dl, and
c) continuing to periodically administer the GnRH antagonist to the patient for the remainder of the treatment period

In another aspect (“A34”), the disclosure features a method of (i) reducing the volume of one or more rectovaginal endometriosis nodes in a human patient diagnosed as having rectovaginal endometriosis, (ii) reducing the volume of one or more type III rectovaginal endometriosis nodes in a human patient diagnosed as having rectovaginal endometriosis, (iii) reducing pelvic pain in a human patient diagnosed as having rectovaginal endometriosis, (iv) reducing dysmenorrhea in a human patient diagnosed as having rectovaginal endometriosis, (v) reducing dyspareunia in a human patient diagnosed as having rectovaginal endometriosis, (vi) reducing dyschezia in a human patient diagnosed as having rectovaginal endometriosis, (vii) reducing uterine bleeding in a human patient diagnosed as having rectovaginal endometriosis, (viii) inducing amenorrhea in a human patient diagnosed as having rectovaginal endometriosis, and/or (ix) and/or (viii) reducing the concentration of FSH, LH, or E2 in a human patient diagnosed as having rectovaginal endometriosis by:
a) periodically administering to the patient a therapeutically effective amount of a GnRH antagonist during a treatment period,
b) monitoring the patient's low-density lipoprotein-cholesterol level, and, if the patient does not exhibit an increase in low-density lipoprotein-cholesterol level from a level less than 160 mg/dl (e.g., from a level less than 130 mg/dl) to a level greater than 190 mg/dl,
c) continuing to periodically administer the GnRH antagonist to the patient for the remainder of the treatment period.

For example, in some embodiments of this aspect, the method includes:
a) periodically administering to the patient a therapeutically effective amount of a GnRH antagonist during a treatment period,
b) determining that the patient does not exhibit an increase in low-density lipoprotein-cholesterol level from a level less than 160 mg/dl (e.g., from a level less than 130 mg/dl) to a level greater than 190 mg/dl, and
c) continuing to periodically administer the GnRH antagonist to the patient for the remainder of the treatment period.

In another aspect (“A35”), the disclosure features a method of (i) reducing the volume of one or more rectovaginal endometriosis nodes in a human patient diagnosed as having rectovaginal endometriosis, (ii) reducing the volume of one or more type III rectovaginal endometriosis nodes in a human patient diagnosed as having rectovaginal endometriosis, (iii) reducing pelvic pain in a human patient diagnosed as having rectovaginal endometriosis, (iv) reducing dysmenorrhea in a human patient diagnosed as having rectovaginal endometriosis, (v) reducing dyspareunia in a human patient diagnosed as having rectovaginal endometriosis, (vi) reducing dyschezia in a human patient diagnosed as having rectovaginal endometriosis, (vii) reducing uterine bleeding in a human patient diagnosed as having rectovaginal endometriosis, (viii) inducing amenorrhea in a human patient diagnosed as having rectovaginal endometriosis, and/or (ix) and/or (viii) reducing the concentration of FSH, LH, or E2 in a human patient diagnosed as having rectovaginal endometriosis by:
a) periodically administering to the patient a therapeutically effective amount of a GnRH antagonist during a treatment period,
b) monitoring the patient's serum triglyceride level, and, if the patient does not exhibit an increase in serum triglyceride level of greater than 30% relative to a measurement of the patient's serum triglyceride level obtained prior to the treatment period (i.e., prior to the onset of treatment with the GnRH antagonist),
c) continuing to periodically administer the GnRH antagonist to the patient for the remainder of the treatment period.
   For example, in some embodiments of this aspect, the method includes:
a) periodically administering to the patient a therapeutically effective amount of a GnRH antagonist during a treatment period,
b) determining that the patient does not exhibit an increase in serum triglyceride level of greater than 30% relative to a measurement of the patient's serum triglyceride level obtained prior to the treatment period (i.e., prior to the onset of treatment with the GnRH antagonist), and
c) continuing to periodically administer the GnRH antagonist to the patient for the remainder of the treatment period.

In another aspect (“A36”), the disclosure features a method of (i) reducing the volume of one or more rectovaginal endometriosis nodes in a human patient diagnosed as having rectovaginal endometriosis, (ii) reducing the volume of one or more type III rectovaginal endometriosis nodes in a human patient diagnosed as having rectovaginal endometriosis, (iii) reducing pelvic pain in a human patient diagnosed as having rectovaginal endometriosis, (iv) reducing dysmenorrhea in a human patient diagnosed as having rectovaginal endometriosis, (v) reducing dyspareunia in a human patient diagnosed as having rectovaginal endometriosis, (vi) reducing dyschezia in a human patient diagnosed as having rectovaginal endometriosis, (vii) reducing uterine bleeding in a human patient diagnosed as having rectovaginal endometriosis, (viii) inducing amenorrhea in a human patient diagnosed as having rectovaginal endometriosis, and/or (ix) and/or (viii) reducing the concentration of FSH, LH, or E2 in a human patient diagnosed as having rectovaginal endometriosis by:
a) periodically administering to the patient a therapeutically effective amount of a GnRH antagonist during a treatment period,
b) monitoring the patient's serum triglyceride level, and, if the patient does not exhibit an increase in serum triglyceride level to a level greater than 200 mg/dl,
c) continuing to periodically administer the GnRH antagonist to the patient for the remainder of the treatment period.
   For example, in some embodiments of this aspect, the method includes:
a) periodically administering to the patient a therapeutically effective amount of a GnRH antagonist during a treatment period,
b) determining that the patient does not exhibit an increase in serum triglyceride level to a level greater than 200 mg/dl, and
c) continuing to periodically administer the GnRH antagonist to the patient for the remainder of the treatment period.

In another aspect (“A37”), the disclosure features a method of (i) reducing the volume of one or more rectovaginal endometriosis nodes in a human patient diagnosed as having rectovaginal endometriosis, (ii) reducing the volume of one or more type III rectovaginal endometriosis nodes in a human patient diagnosed as having rectovaginal endometriosis, (iii) reducing pelvic pain in a human patient diagnosed as having rectovaginal endometriosis, (iv) reducing dysmenorrhea in a human patient diagnosed as having rectovaginal endometriosis, (v) reducing dyspareunia in a human patient diagnosed as having rectovaginal endometriosis, (vi) reducing dyschezia in a human patient diagnosed as having rectovaginal endometriosis, (vii) reducing uterine bleeding in a human patient diagnosed as having rectovaginal endometriosis, (viii) inducing amenorrhea in a human patient diagnosed as having rectovaginal endometriosis, and/or (ix) and/or (viii) reducing the concentration of FSH, LH, or E2 in a human patient diagnosed as having rectovaginal endometriosis by:
a) periodically administering to the patient a therapeutically effective amount of a GnRH antagonist during a treatment period,
b) monitoring the patient's serum triglyceride level, and, if the patient does not exhibit an increase in serum triglyceride level from a level less than 300 mg/dl to a level greater than 500 mg/dl,
c) continuing to periodically administer the GnRH antagonist to the patient for the remainder of the treatment period.
   For example, in some embodiments of this aspect, the method includes:
a) periodically administering to the patient a therapeutically effective amount of a GnRH antagonist during a treatment period,
b) determining that the patient does not exhibit an increase in serum triglyceride level from a level less than 300 mg/dl to a level greater than 500 mg/dl, and
c) continuing to periodically administer the GnRH antagonist to the patient for the remainder of the treatment period.

In some embodiments of any of the above aspects (A1-A37) or embodiments thereof, the treatment period has a duration of from about 2 weeks to about 2 years, or more. In some embodiments, the treatment period has a duration of from about 4 weeks to about 52 weeks. In some embodiments, the treatment period has a duration of from about 4 weeks to about 48 weeks. In some embodiments, the treatment period has a duration of from about 4 weeks to about 44 weeks. In some embodiments, the treatment period has a duration of from about 4 weeks to about 40 weeks. In some embodiments, the treatment period has a duration of from about 4 weeks to about 36 weeks. In some embodiments, the treatment period has a duration of from about 4 weeks to about 32 weeks. In some embodiments, the treatment period has a duration of from about 4 weeks to about 28 weeks. In some embodiments, the treatment period has a duration of from about 4 weeks to about 24 weeks. In some embodiments, the treatment period has a duration of from about 4 weeks to about 20 weeks. In some embodiments, the treatment period has a duration of from about 4 weeks to about 16 weeks. In some embodiments, the treatment period has a duration of from about 4 weeks to about 12 weeks. In some embodiments, the treatment period has a duration of from about 4 weeks to about 8 weeks. In some embodiments, the treatment period has a duration of at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least 51 weeks, at least 52 weeks, at least 53 weeks, at least 54 weeks, at least 55 weeks, at least 56 weeks, at least 57 weeks, at least 58 weeks, at least 59 weeks, at least 60 weeks, at least 61 weeks, at least 62 weeks, at least 63 weeks, at least 64 weeks, at least 65 weeks, at least 66 weeks, at least 67 weeks, at least 68 weeks, at least 69 weeks, at least 70 weeks, at least 71 weeks, at least 72 weeks, at least 73 weeks, at least 74 weeks, at least 75 weeks, at least 76 weeks, at least 77 weeks, at least 78 weeks, at least 79 weeks, at least 80 weeks, at least 81 weeks, at least 82 weeks, at least 83 weeks, at least 84 weeks, at least 85 weeks, at least 86 weeks, at least 87 weeks, at least 88 weeks, at least 89 weeks, at least 90 weeks, at least 91 weeks, at least 92 weeks, at least 93 weeks, at least 94 weeks, at least 95 weeks, at least 96 weeks, at least 97 weeks, at least 98 weeks, at least 99 weeks, at least 100 weeks, at least 101 weeks, at least 102 weeks, at least 103 weeks, at least 104 weeks, at least 105 weeks, at least 106 weeks, at least 107 weeks, at least 108 weeks, at least 109 weeks, at least 110 weeks, at least 111 weeks, at least 112 weeks, at least 113 weeks, at least 114 weeks, at least 115 weeks, at least 116 weeks, at least 117 weeks, at least 118 weeks, at least 119 weeks, at least 120 weeks, at least 121 weeks, at least 122 weeks, at least 123 weeks, at least 124 weeks, at least 125 weeks, at least 126 weeks, at least 127 weeks, at least 128 weeks, at least 129 weeks, at least 130 weeks, at least 131 weeks, at least 132 weeks, at least 133 weeks, at least 134 weeks, at least 135 weeks, at least 136 weeks, at least 137 weeks, at least 138 weeks, at least 139 weeks, at least 140 weeks, at least 141 weeks, at least 142 weeks, at least 143 weeks, at least 144 weeks, at least 145 weeks, at least 146 weeks, at least 147 weeks, at least 148 weeks, at least 149 weeks, at least 150 weeks, at least 151 weeks, at least 152 weeks, at least 153 weeks, at least 154 weeks, at least 155 weeks, at least 156 weeks, at least 157 weeks, at least 158 weeks, at least 159 weeks, at least 160 weeks, at least 161 weeks, at least 162 weeks, at least 163 weeks, at least 164 weeks, at least 165 weeks, at least 166 weeks, at least 167 weeks, at least 168 weeks, at least 169 weeks, at least 170 weeks, at least 171 weeks, at least 172 weeks, at least 173 weeks, at least 174 weeks, at least 175 weeks, at least 176 weeks, at least 177 weeks, at least 178 weeks, at least 179 weeks, at least 180 weeks, at least 181 weeks, at least 182 weeks, at least 183 weeks, at least 184 weeks, at least 185 weeks, at least 186 weeks, at least 187 weeks, at least 188 weeks, at least 189 weeks, at least 190 weeks, at least 191 weeks, at least 192 weeks, at least 193 weeks, at least 194 weeks, at least 195 weeks, at least 196 weeks, at least 197 weeks, at least 198 weeks, at least 199 weeks, at least 200 weeks, at least 201 weeks, at least 202 weeks, at least 203 weeks, at least 204 weeks, at least 205 weeks, at least 206 weeks, at least 207 weeks, at least 208 weeks, at least 209 weeks, at least 210 weeks, at least 211 weeks, at least 212 weeks, at least 213 weeks, at least 214 weeks, at least 215 weeks, at least 216 weeks, at least 217 weeks, at least 218 weeks, at least 219 weeks, at least 220 weeks, at least 221 weeks, at least 222 weeks, at least 223 weeks, at least 224 weeks, at least 225 weeks, at least 226 weeks, at least 227 weeks, at least 228 weeks, at least 229 weeks, at least 230 weeks, at least 231 weeks, at least 232 weeks, at least 233 weeks, at least 234 weeks, at least 235 weeks, at least 236 weeks, at least 237 weeks, at least 238 weeks, at least 239 weeks, at least 240 weeks, at least 241 weeks, at least 242 weeks, at least 243 weeks, at least 244 weeks, at least 245 weeks, at least 246 weeks, at least 247 weeks, at least 248 weeks, at least 249 weeks, at least 250 weeks, at least 251 weeks, at least 252 weeks, at least 253 weeks, at least 254 weeks, at least 255 weeks, at least 256 weeks, at least 257 weeks, at least 258 weeks, at least 259 weeks, at least 260 weeks, or more.

In some embodiments, the treatment period has a duration of at least about 12 months, such as a duration of at least 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, or more.

In some embodiments, the treatment period has a duration of from about 12 months to about 60 months, such as a duration of from about 12 months to about 59 months, from about 12 months to about 58 months, from about 12 months to about 57 months, from about 12 months to about 56 months, from about 12 months to about 55 months, from about 12 months to about 54 months, from about 12 months to about 53 months, from about 12 months to about 52 months, from about 12 months to about 51 months, from about 12 months to about 50 months, from about 12 months to about 49 months, from about 12 months to about 48 months, from about 12 months to about 47 months, from about 12 months to about 46 months, from about 12 months to about 45 months, from about 12 months to about 44 months, from about 12 months to about 43 months, from about 12 months to about 42 months, from about 12 months to about 41 months, from about 12 months to about 40 months, from about 12 months to about 39 months, from about 12 months to about 38 months, from about 12 months to about 37 months, from about 12 months to about 36 months, from about 12 months to about 35 months, from about 12 months to about 34 months, from about 12 months to about 33 months, from about 12 months to about 32 months, from about 12 months to about 31 months, from about 12 months to about 30 months, from about 12 months to about 29 months, from about 12 months to about 28 months, from about 12 months to about 27 months, from about 12 months to about 26 months, from about 12 months to about 25 months, from about 12 months to about 24 months, from about 12 months to about 23 months, from about 12 months to about 22 months, from about 12 months to about 21 months, from about 12 months to about 20 months, from about 12 months to about 19 months, or from about 12 months to about 18 months, among other lengths.

In some embodiments, the treatment period has a duration of about 24 weeks, 25 weeks, 26 weeks, 27 weeks, 28 weeks, 29 weeks, 30 weeks, 31 weeks, 32 wees, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or more.

In some embodiments of the disclosure, the GnRH antagonist is a compound represented by formula (I)

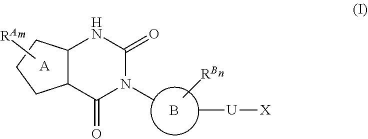

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof.

In some embodiments of formula (I), the ring A is a thiophene ring represented by formula (IIa)

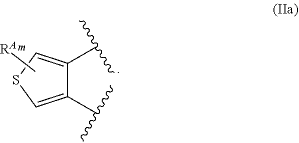

In some embodiments of formula (I) or (IIa), m is 1.

In some embodiments of formula (I) or (IIa), the ring A is an optionally substituted thiophene ring represented by formula (IIb)

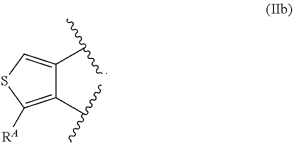

In some embodiments of formula (I), (IIa), or (IIb), each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

In some embodiments of formula (I), (IIa), or (IIb), each R^{A} is COOH or pharmaceutically acceptable salt thereof.

In some embodiments of formula (I), (IIa), or (IIb), the ring B is an optionally substituted benzene ring, pyridine ring, or thiophene ring.

In some embodiments of formula (I), (IIa), or (IIb), the ring B is represented by a formula selected from the group consisting of:

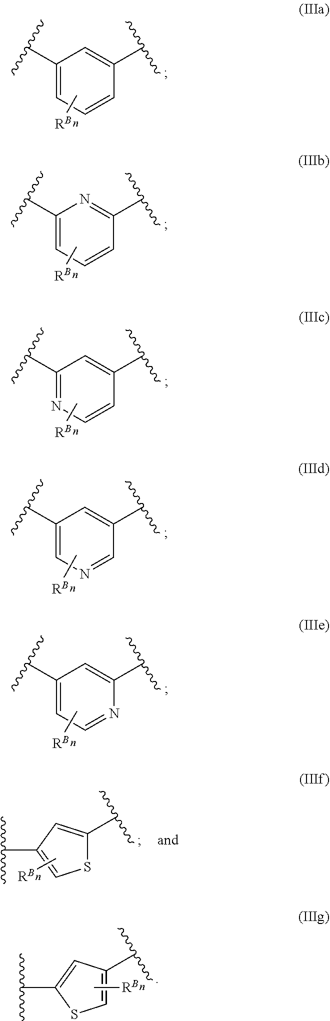

In some embodiments of formula (I), (IIa), (IIb), or any one of (IIIa)-(IIIg), n is 2.

In some embodiments of formula (I), (IIa), (IIb), or any one of (IIIa)-(IIIg), the ring B is represented by a formula selected from the group consisting of:

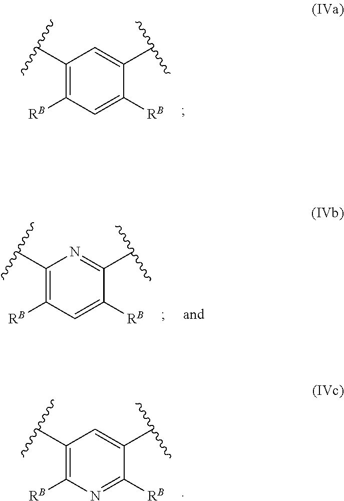

In some embodiments of formula (I), (IIa), (IIb), any one of (IIIa)-(IIIg), or any one of (IVa)-(IVc), each R^{B} is independently a halogen atom, an optionally substituted lower alkyl group, or OW⁴, wherein each W⁴ is independently a hydrogen atom or an optionally substituted lower alkyl group.

In some embodiments of formula (I), (IIa), (IIb), any one of (IIIa)-(IIIg), or any one of (IVa)-(IVc), each R^{B} is independently a fluorine atom, chlorine atom, bromine atom, methyl group, or methoxy group.

In some embodiments of formula (I), (IIa), (IIb), any one of (IIIa)-(IIIg), or any one of (IVa)-(IVc), U is a single bond.

In some embodiments of formula (I), (IIa), (IIb), any one of (IIIa)-(IIIg), or any one of (IVa)-(IVc), X is a group represented by —O-L-Y.

In some embodiments of formula (I), (IIa), (IIb), any one of (IIIa)-(IIIg), or any one of (IVa)-(IVc), L is a methylene group.

In some embodiments of formula (I), (IIa), (IIb), any one of (IIIa)-(IIIg), or any one of (IVa)-(IVc), Y is an optionally substituted benzene ring represented by formula (V)

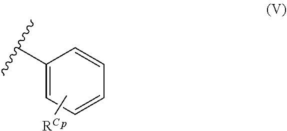

wherein each R^{C} is independently a halogen atom, an optionally substituted lower alkyl group, or OW⁹, wherein each W⁹ is independently a hydrogen atom or an optionally substituted lower alkyl group; and

p is an integer from 0 to 3.

In some embodiments of formula (I), (IIa), (IIb), any one of (IIIa)-(IIIg), any one of (IVa)-(IVc), or (V), Y is a substituted benzene ring represented by formula (Va)

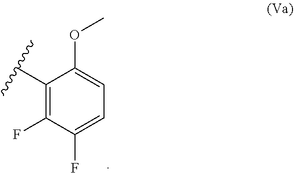 [SMILES: *c1c(OC)ccc(F)c1F]

In some embodiments, the compound is represented by formula (Ia)

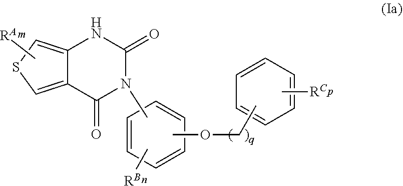

wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

q is an integer from 0 to 3;

each R^{C} is independently a halogen atom, an optionally substituted lower alkyl group, or OW⁹, wherein each W⁹ is independently a hydrogen atom or an optionally substituted lower alkyl group; and

p is an integer from 0 to 3;

or a pharmaceutically acceptable salt thereof.

In some embodiments, the compound is represented by formula (Ib)

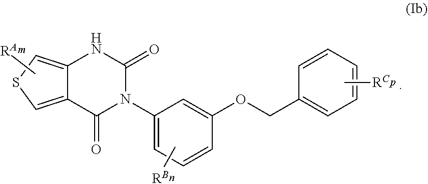

In some embodiments, the compound is represented by formula (Ic)

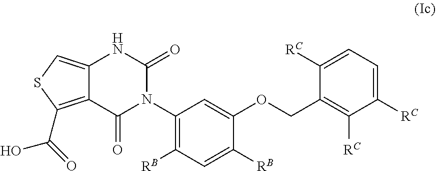

or a pharmaceutically acceptable salt thereof.

In some embodiments, the compound is 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid, represented by formula (VI)

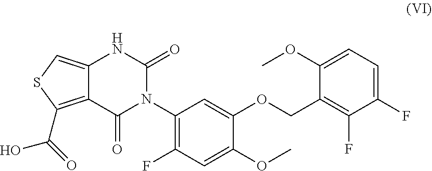 [SMILES: COc1cc(F)c(-n2c(=O)[nH]c3csc(C(=O)O)c3c2=O)cc1OCc1c(OC)ccc(F)c1F]

or a pharmaceutically acceptable salt thereof.

In some embodiments, the compound is the choline salt of the compound represented by formula (VI), choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate. It is to be understood that references herein to a compound represented by formula (VI) specifically include the choline salt of compound (VI), which is represented by formula (VIa), below.

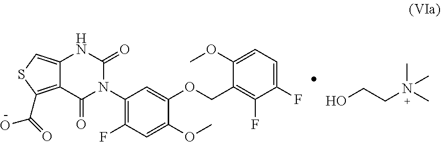

In some embodiments, the choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate is in a crystalline state.

In some embodiments, the choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate exhibits characteristic X-ray powder diffraction (XRPD) peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ.

In some embodiments, the choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm.

In some embodiments, the choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate exhibits ¹⁹F solid-state NMR peaks centered at about −151.8 ppm, −145.2 ppm, and −131.6 ppm.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is orally administered to the patient.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 25 mg to about 400 mg per dose during the treatment period (e.g., in the recited amount or in an equivalent amount of a pharmaceutically acceptable salt, such as a choline salt), such as an amount of about 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, or 400 mg of the compound of any one of formulas (I), (Ia)-(Ic), (IIa), (IIb), (IIIa)-(IIIg), (IVa)-(IVc), (V), (Va), or (VI) during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 35 mg to about 65 mg per dose during the treatment period (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per dose during the treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 50 mg per dose during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 60 mg to about 90 mg per dose during the treatment period (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per dose during the treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 75 mg per dose during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 50 mg to about 150 mg per dose during the treatment period (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per dose during the treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 100 mg per dose during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 150 mg to about 250 mg per dose during the treatment period (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per dose during the treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 200 mg per dose during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more doses (i.e., one or more times) per day, week, or month during the treatment period, such as from 1 to 10 times per day during the treatment period (e.g., 1 time, 2 times, 3 times, 4 times, 5 times, 6 times, 7 times, 8 times, 9 times, or 10 times per day, such as 1 time or 2 times per day), 1 to 100 times per week during the treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the treatment period, the compound of any one of formulas (I), (Ia)-(Ic), (IIa), (IIb), (IIIa)-(IIIg), (IVa)-(IVc), (V), (Va), or (VI) (e.g., (VIa)) may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more doses per day during the treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (I), (Ia)-(Ic), (IIa), (IIb), (IIIa)-(IIIg), (IVa)-(IVc), (V), (Va), or (VI) (e.g., (VIa)) may be administered to the patient in one or more unit dosage forms that collectively constitute a single dose. For example, at a given time within the treatment period, a patient may be administered a single dose of the compound of a specified amount, such as a single dose of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, or more (e.g., in the recited amount or in an equivalent amount of a pharmaceutically acceptable salt, such as a choline salt), by administration of one or more unit dosage forms of the compound to the patient. As a non-limiting example, a single dose of 200 mg of the compound may be administered to the subject by way of two individual 100-mg unit dosage forms of the compound. The two 100-mg unit dosage forms collectively constitute a single 200-mg dose of the compound if administered to the patient at substantially the same time.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 35 mg to about 65 mg per day during the treatment period (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per day during the treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 50 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 60 mg to about 90 mg per day during the treatment period (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per day during the treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 75 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 50 mg to about 150 mg per day during the treatment period (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per day during the treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 100 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 150 mg to about 250 mg per day during the treatment period (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per day during the treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 200 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in a single dose per day during the treatment period.

For example, the compound may be administered to the patient in an amount (e.g., a single dose) of from about 35 mg to about 65 mg per day during the treatment period (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per day during the treatment period (e.g., in the recited amount or in an equivalent amount of a pharmaceutically acceptable salt, such as a choline salt). In some embodiments, the compound is administered to the patient in an amount (e.g., a single dose) of about 50 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount (e.g., a single dose) of from about 60 mg to about 90 mg per day during the treatment period (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per day during the treatment period (e.g., in the recited amount or in an equivalent amount of a pharmaceutically acceptable salt, such as a choline salt). In some embodiments, the compound is administered to the patient in an amount (e.g., a single dose) of about 75 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount (e.g., a single dose) of from about 50 mg to about 150 mg per day during the treatment period (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per day during the treatment period (e.g., in the recited amount or in an equivalent amount of a pharmaceutically acceptable salt, such as a choline salt). In some embodiments, the compound is administered to the patient in an amount (e.g., a single dose) of about 100 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount (e.g., a single dose) of from about 150 mg to about 250 mg per day during the treatment period (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per day during the treatment period (e.g., in the recited amount or in an equivalent amount of a pharmaceutically acceptable salt, such as a choline salt). In some embodiments, the compound is administered to the patient in an amount (e.g., a single dose) of about 200 mg per day during the treatment period (e.g., in the recited amount or in an equivalent amount of a pharmaceutically acceptable salt, such as a choline salt).

In some embodiments of the disclosure, the GnRH antagonist is a compound represented by any one of formulas (VII)-(XIV), below, such as elagolix, relugolix, or opigolix (ASP1707). In some embodiments, the GnRH antagonist is BAY-784 or SK-2706.

For example, in some embodiments, the GnRH antagonist is a compound represented by formula

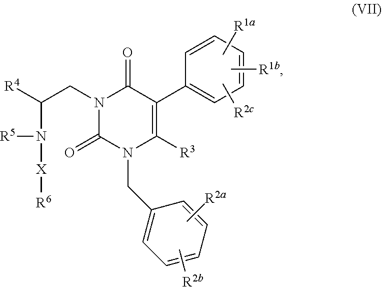

wherein R₁ₐ, R_{1b} and R_{1c} are the same or different and are each independently hydrogen, halogen, C₁₋₄alkyl, hydroxy or alkoxy, or R₁ₐ and R_{1b} taken together form —OCH₂O— or —OCH₂CH₂—;

R₂ₐ and R_{2b} are the same or different and are each independently hydrogen, halogen, trifluoromethyl, cyano or —SO₂CH₃;

R₃ is hydrogen or methyl;

R₄ is phenyl or C₃₋₇alkyl;

R₅ is hydrogen or C₁₋₄alkyl;

R₆ is —COOH or an acid isostere; and

X is C₁₋₆alkanediyl optionally substituted with from 1 to 3 C₁₋₆alkyl groups;

or a pharmaceutically acceptable salt thereof.

In some embodiments, the GnRH antagonist is a compound represented by formula (VIII)

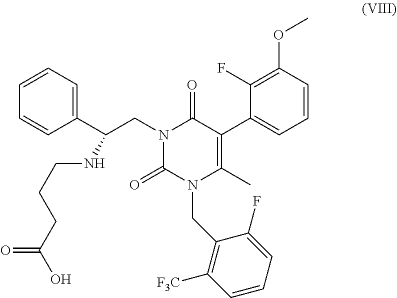 [SMILES: COc1cccc(-c2c(C)n(Cc3c(F)cccc3C(F)(F)F)c(=O)n(C[C@H](NCCCC(=O)O)c3ccccc3)c2=O)c1F]

or a pharmaceutically acceptable salt thereof. In some embodiments, the GnRH antagonist is the sodium salt of the compound represented by formula (VIII), which is represented by formula (IX), below.

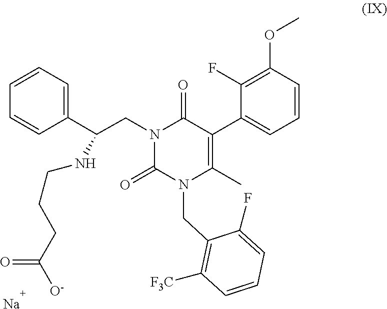

In some embodiments, the compound of any one of formulas (VII)-(IX) is administered to the patient in an amount of from about 50 mg to about 650 mg per dose during the treatment period (e.g., in the recited amount or in an equivalent amount of a pharmaceutically acceptable salt, such as a sodium salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, or 650 mg per dose during the treatment period (e.g., in the recited amount or in an equivalent amount of a pharmaceutically acceptable salt, such as a sodium salt). In some embodiments, the compound of any one of formulas (VII)-(IX) is administered to the patient in an amount of about 150 mg per dose during the treatment period, 300 mg per dose during the treatment period, 400 mg per dose during the treatment period, or 600 mg per dose during the treatment period (e.g., in the recited amount or in an equivalent amount of a pharmaceutically acceptable salt, such as a sodium salt).

In some embodiments, the compound of any one of formulas (VII)-(IX) is administered to the patient in one or more doses (i.e., one or more times) per day, week, or month during the treatment period, such as from 1 to 10 times per day during the treatment period (e.g., 1 time, 2 times, 3 times, 4 times, 5 times, 6 times, 7 times, 8 times, 9 times, or 10 times per day, such as 1 time or 2 times per day), 1 to 100 times per week during the treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the treatment period, the compound of any one of formulas (VII)-(IX) may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (VII)-(IX) is administered to the patient in one or more doses per day during the treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (VII)-(IX) may be administered to the patient in one or more unit dosage forms that collectively constitute a single dose. For example, a patient may be administered a single dose of the compound of a specified amount, such as a single dose of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, or more (e.g., in the recited amount or in an equivalent amount of a pharmaceutically acceptable salt, such as a sodium salt), by administration of one or more unit dosage forms of the compound to the patient. As a non-limiting example, a single dose of 300 mg of the compound may be administered to the subject by way of two individual 150-mg unit dosage forms of the compound. The two 150-mg unit dosage forms collectively constitute a single 300-mg dose of the compound if administered to the patient at substantially the same time.

In some embodiments, the compound of any one of formulas (VII)-(IX) is administered to the patient in one or more daily doses (e.g., from 1 to 10 doses, such as 1, 2, 3, 4, 5, 6, 7, 8, 9, or 10 doses, or more) totaling from about 50 mg to about 650 mg per day during the treatment period (e.g., in the recited amount or in an equivalent amount of a pharmaceutically acceptable salt, such as a sodium salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, or 650 mg per day during the treatment period (e.g., in the recited amount or in an equivalent amount of a pharmaceutically acceptable salt, such as a sodium salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 150 mg per day during the treatment period, 300 mg per day during the treatment period, 400 mg per day during the treatment period (e.g., 200 mg administered twice daily), or 600 mg per day during the treatment period (e.g., 300 mg administered twice daily) (e.g., in the recited amount or in an equivalent amount of a pharmaceutically acceptable salt, such as a sodium salt).

In some embodiments, the GnRH antagonist is a compound represented by formula (X)

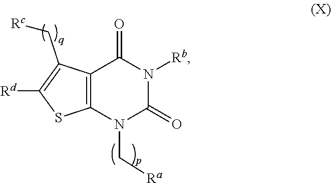

wherein R^{a} is a hydrogen atom, an optionally substituted aryl group (such as an aryl group that may have 1 to 5 substituents selected from halogen, nitro, cyano, amino, a carboxyl group that may be esterified or amidated, an alkylenedioxy, alkyl, alkoxy, alkylthio, alkylsulfinyl, and alkylsulfonyl), an optionally substituted cycloalkyl group, or an optionally substituted heterocyclic group;

R^{b} is an optionally substituted nitrogen-containing heterocyclic group;

R^{c} is an optionally substituted amino group;

R^{d} is an optionally substituted aryl group;

p is an integer from 0 to 3; and

q is an integer from 0 to 3;

or a pharmaceutically acceptable salt thereof.

In some embodiments, the GnRH antagonist is a compound represented by formula (XI)

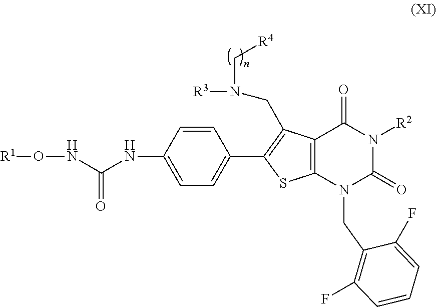

wherein R¹ is C₁₋₄alkyl;

R² is (1) a C₁₋₆alkyl which may have a substituent selected from the group consisting of (1′) a hydroxy group, (2′) a C₁₋₄alkoxy, (3′) a C₁₋₄alkoxy-carbonyl, (4′) a di-C₁₋₄alkyl-carbamoyl, (5′) a 5- to 7-membered nitrogen-containing heterocyclic group, (6′) a C₁₋₄alkyl-carbonyl and (7′) a halogen, (2) a C₃₋₈ cycloalkyl which may have (1′) a hydroxy group or (2′) a mono-C₁₋₄alkyl-carbonylamino, (3) a 5- to 7-membered nitrogen-containing heterocyclic group which may have a substituent selected from the group consisting of (1′) a halogen, (2′) a hydroxy group, (3′) a C₁₋₄alkyl and (4′) a C₁₋₄alkoxy, (4) a phenyl which may have a substituent selected from the group consisting of (1′) a halogen, (2′) a C₁₋₄ alkoxy-C₁₋₄ alkyl, (3′) a mono-C₁₋₄alkyl-carbamoyl-C₁₋₄alkyl, (4′) a C₁₋₄alkoxy and (5′) a mono-C₁₋₄alkylcarbamoyl-C₁₋₄alkoxy, or (5) a C₁₋₄alkoxy;

R³ is C₁₋₄alkyl;

R⁴ is (1) hydrogen, (2) C₁₋₄alkoxy, (3) C₆₋₁₀aryl, (4) N—C₁₋₄alkyl-N—C₁₋₄alkylsulfonylamino, (5) hydroxyl, or (6) a 5- to 7-membered nitrogen-containing heterocyclic group which may have a substituent selected from the group consisting of (1′) oxo, (2′) a C₁₋₄alkyl, (3′) a hydroxy-C₁₋₄alkyl, (4′) a C₁₋₄alkoxy-carbonyl, (5′) a mono-C₁₋₄alkyl-carbamoyl and (6′) a C₁₋₄alkylsulfonyl; and

n is an integer from 1 to 4;

optionally provided that when R² is a phenyl which may have a substituent, R⁴ is a 5- to 7-membered nitrogen-containing heterocyclic group which may have a substituent selected from the group consisting of (1) oxo, (2) hydroxy-C₁₋₄alkyl, (3) C₁₋₄alkoxy-carbonyl, (4) mono-C₁₋₄alkyl-carbamoyl and (5) C₁₋₄alkylsulfonyl;

or a pharmaceutically acceptable salt thereof. In some embodiments, the GnRH antagonist is a compound represented by formula (XII), below.

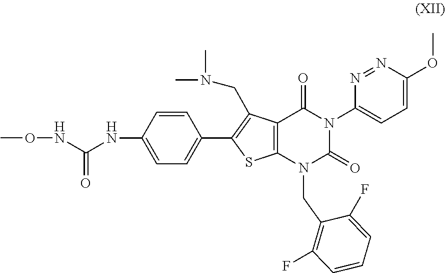

In some embodiments, the compound of any one of formulas (X)-(XII) is administered to the patient in an amount of from about 10 mg to about 60 mg per dose during the treatment period (e.g., in the recited amount or in an equivalent amount of a pharmaceutically acceptable salt, such as a chlorine salt), such as an amount of about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg per dose during the treatment period (e.g., in the recited amount or in an equivalent amount of a pharmaceutically acceptable salt, such as a chlorine salt). In some embodiments, the compound of any one of formulas (X)-(XII) is administered to the patient in an amount of about 40 mg per dose during the treatment period (e.g., in the recited amount or in an equivalent amount of a pharmaceutically acceptable salt, such as a chlorine salt).

In some embodiments, the compound of any one of formulas (X)-(XII) is administered to the patient in one or more doses (i.e., one or more times) per day, week, or month during the treatment period, such as from 1 to 10 times per day during the treatment period (e.g., 1 time, 2 times, 3 times, 4 times, 5 times, 6 times, 7 times, 8 times, 9 times, or 10 times per day, such as 1 time or 2 times per day), 1 to 100 times per week during the treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the treatment period, the compound of any one of formulas (X)-(XII) may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (X)-(XII) is administered to the patient in one or more doses per day during the treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (I)-(XII) may be administered to the patient in one or more unit dosage forms that collectively constitute a single dose. For example, a patient may be administered a single dose of the compound of a specified amount, such as a single dose of 25 mg, 50 mg, 75 mg, 100 mg, or more (e.g., in the recited amount or in an equivalent amount of a pharmaceutically acceptable salt, such as a chlorine salt), by administration of one or more unit dosage forms of the compound to the patient. As a non-limiting example, a single dose of 40 mg of the compound may be administered to the subject by way of two individual 20-mg unit dosage forms of the compound. The two 20-mg unit dosage forms collectively constitute a single 40-mg dose of the compound if administered to the patient at substantially the same time.

In some embodiments, the compound of any one of formulas (X)-(XII) is administered to the patient in one or more daily doses (e.g., from 1 to 10 doses, such as 1, 2, 3, 4, 5, 6, 7, 8, 9, or 10 doses, or more) totaling from about 10 mg to about 60 mg per day during the treatment period (e.g., in the recited amount or in an equivalent amount of a pharmaceutically acceptable salt, such as a chlorine salt), such as an amount of about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg per day during the treatment period (e.g., in the recited amount or in an equivalent amount of a pharmaceutically acceptable salt, such as a chlorine salt). In some embodiments, the compound is administered to the patient in one or more daily doses (e.g., from 1 to 10 doses, such as 1, 2, 3, 4, 5, 6, 7, 8, 9, or 10 doses, or more) totaling from about 20 mg to about 50 mg per day during the treatment period (e.g., in the recited amount or in an equivalent amount of a pharmaceutically acceptable salt, such as a chlorine salt), such as an amount of about 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg per day during the treatment period (e.g., in the recited amount or in an equivalent amount of a pharmaceutically acceptable salt, such as a chlorine salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 40 mg per day during the treatment period (e.g., a single daily dose of 40 mg) (e.g., in the recited amount or in an equivalent amount of a pharmaceutically acceptable salt, such as a chlorine salt).

In some embodiments, the GnRH antagonist is a compound represented by formula (XIII)

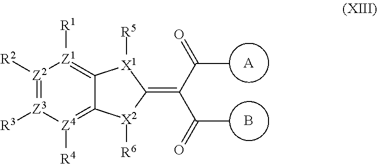

wherein R¹, R², R³ and R⁴ are the same or different, and are each independently selected from hydrogen, nitro, cyano, halogen, an optionally substituted hydrocarbon group, an optionally substituted heterocyclic group, hydroxy, alkoxy, carboxy, optionally substituted acyl-O—, optionally substituted acyl, a substituent —S(O)n₁₀₁- (wherein n₁₀₁ is an integer of 0 to 2), H—S(O)n₁₀₁-, optionally substituted carbamoyl, optionally substituted sulfamoyl, optionally substituted amino, and two adjacent groups selected from the group of R¹, R², R³ and R⁴ may combine to form an aryl or a carbocyclic (e.g., cycloalkenyl) group;

R⁵ and R⁶ are the same or different and are each independently selected from hydrogen, halogen, optionally substituted hydrocarbon, and optionally substituted amino,

X¹ and X² are the same or different and are each independently selected from N, S and O;

A and B are the same or different and are each independently selected from optionally substituted aryl and optionally substituted heterocyclyl, and

Z¹, Z², Z³ and Z⁴ are each independently selected from C and N; optionally provided that 1) when X¹ and X² each is S or O, one or both of the corresponding R⁵ and R⁶ are absent; and/or 2) when one to four of Z¹, Z², Z³ and/or Z⁴ are N, the corresponding R¹, R², R³ and/or R⁴ are absent;

or a pharmaceutically acceptable salt thereof. In some embodiments, the GnRH antagonist is a compound represented by formula (XIV), below.

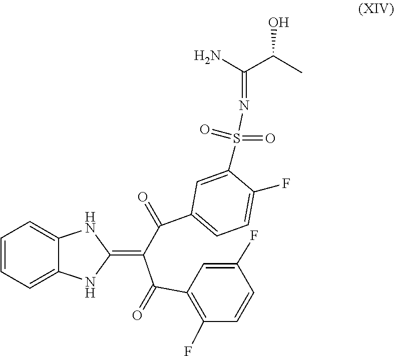 [SMILES: C[C@@H](O)/C(N)=N/S(=O)(=O)c1cc(C(=O)C(C(=O)c2cc(F)ccc2F)=C2Nc3ccccc3N2)ccc1F]

In some embodiments, the compound of any one of formulas (XIII) and (XIV), or SKI2670 or BAY-784, is administered to the patient in one or more doses (i.e., one or more times) per day, week, or month during the treatment period, such as from 1 to 10 times per day during the treatment period (e.g., 1 time, 2 times, 3 times, 4 times, 5 times, 6 times, 7 times, 8 times, 9 times, or 10 times per day, such as 1 time or 2 times per day), 1 to 100 times per week during the treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the treatment period, the compound of any one of formulas (XIII) and (XIV), or SKI2670 or BAY-784, may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (XIII) and (XIV), or SKI2670 or BAY-784, is administered to the patient in one or more doses per day during the treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

Additionally, using the compositions and methods of the disclosure, a patient that is being treated, or that has previously been treated, with a GnRH antagonist that is not a compound represented by formula (I), herein, may undergo a change in therapy so as to be administered a compound of formula (I). The change in therapy to a compound of formula (I) may be made, for example, in view of the surprising and beneficial treatment outcomes that such a compound effectuates (as described, e.g., in the working examples herein).

For example, in a further aspect (“A38”), the disclosure features a method of treating an estrogen-dependent disease, such as uterine fibroids, endometriosis, adenomyosis, or rectovaginal endometriosis, in a human patient in need thereof by periodically administering to the patient (e.g., during a treatment period, such as a treatment period having a duration described herein) a therapeutically effective amount of a GnRH antagonist represented by formula (I)

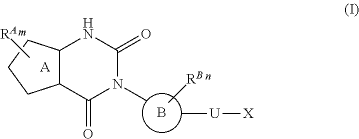

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof,

wherein the patient has previously been administered a GnRH antagonist that is not a compound represented by formula (I). In some embodiments, the GnRH antagonist that has previously been administered to the patient is a compound represented by any one of formulas (VII)-(XIV), herein, such as elagolix, relugolix, or opigolix (ASP1707).

In another aspect (“A39”), the disclosure features a method of reducing the volume of menstrual blood loss, inducing amenorrhea, reducing the volume of one or more uterine fibroids, preserving hemoglobin, and/or reducing the concentration of FSH, LH, or E2 in a human patient diagnosed as having uterine fibroids by periodically administering to the patient (e.g., during a treatment period, such as a treatment period having a duration described herein) a therapeutically effective amount of a GnRH antagonist represented by formula (I)

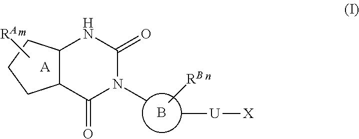

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof,

wherein the patient has previously been administered a GnRH antagonist that is not a compound represented by formula (I). In some embodiments, the GnRH antagonist that has previously been administered to the patient is a compound represented by any one of formulas (VII)-(XIV), herein, such as elagolix, relugolix, or opigolix (ASP1707).

In an additional aspect (“A40”), the disclosure features a method of reducing the volume of one or more endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having endometriosis by periodically administering to the patient (e.g., during a treatment period, such as a treatment period having a duration described herein) a therapeutically effective amount of a GnRH antagonist represented by formula (I)

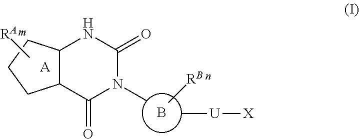

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁸ independently are a hydrogen atom or an optionally substituted lower alkyl group, or W⁶ and W⁸ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof,

wherein the patient has previously been administered a GnRH antagonist that is not a compound represented by formula (I). In some embodiments, the GnRH antagonist that has previously been administered to the patient is a compound represented by any one of formulas (VII)-(XIV), herein, such as elagolix, relugolix, or opigolix (ASP1707).

In another aspect (“A41”), the disclosure features a method of reducing uterine volume, reducing the thickness of the anterior and/or posterior region of the uterine myometrium, reducing pelvic pain, reducing dysmenorrhea, reducing dyspareunia, reducing dyschezia, reducing uterine tenderness, reducing uterine bleeding, inducing amenorrhea, and/or reducing the concentration of FSH, LH, or E2 in a human patient diagnosed as having adenomyosis by periodically administering to the patient (e.g., during a treatment period, such as a treatment period having a duration described herein) a therapeutically effective amount of a GnRH antagonist represented by formula (I)

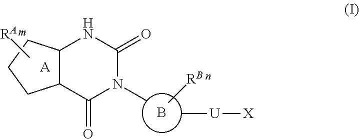

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof,

wherein the patient has previously been administered a GnRH antagonist that is not a compound represented by formula (I). In some embodiments, the GnRH antagonist that has previously been administered to the patient is a compound represented by any one of formulas (VII)-(XIV), herein, such as elagolix, relugolix, or opigolix (ASP1707).

In another aspect (“A42”), the disclosure features a method of reducing the volume of one or more rectovaginal endometriosis nodes, reducing the volume of one or more type III rectovaginal endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having rectovaginal endometriosis by periodically administering to the patient (e.g., during a treatment period, such as a treatment period having a duration described herein) a therapeutically effective amount of a GnRH antagonist represented by formula (I)

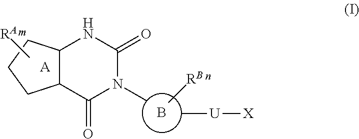

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof,

wherein the patient has previously been administered a GnRH antagonist that is not a compound represented by formula (I). In some embodiments, the GnRH antagonist that has previously been administered to the patient is a compound represented by any one of formulas (VII)-(XIV), herein, such as elagolix, relugolix, or opigolix (ASP1707).

In some embodiments of formula (I), above, the ring A is a thiophene ring represented by formula (IIa)

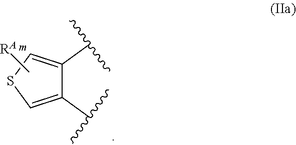

In some embodiments of formula (I) or (IIa), above, m is 1.

In some embodiments of formula (I) or (IIa), above, the ring A is an optionally substituted thiophene ring represented by formula (IIb)

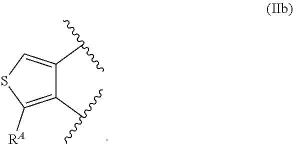

In some embodiments of formula (I), (IIa), or (IIb), above, each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

In some embodiments of formula (I), (IIa), or (IIb), above, each R^{A} is COOH or pharmaceutically acceptable salt thereof.

In some embodiments of formula (I), (IIa), or (IIb), above, the ring B is an optionally substituted benzene ring, pyridine ring, or thiophene ring.

In some embodiments of formula (I), (IIa), or (IIb), above, the ring B is represented by a formula selected from the group consisting of:

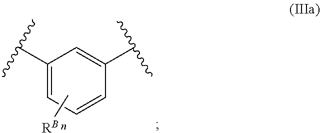

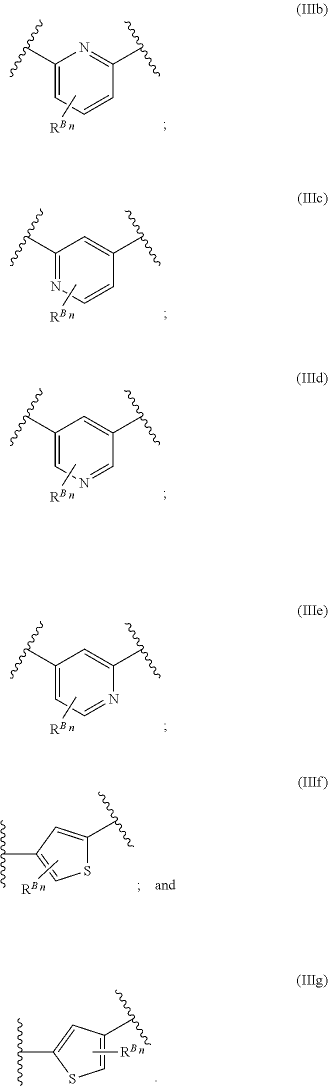

In some embodiments of formula (I), (IIa), (IIb), or any one of (IIIa)-(IIIg), above, n is 2.

In some embodiments of formula (I), (IIa), (IIb), or any one of (IIIa)-(IIIg), above, the ring B is represented by a formula selected from the group consisting of:

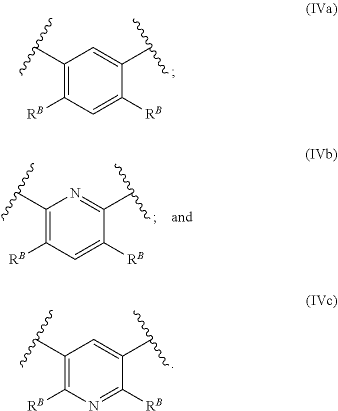

In some embodiments of formula (I), (IIa), (IIb), any one of (IIIa)-(IIIg), or any one of (IVa)-(IVc), above, each R^{B} is independently a halogen atom, an optionally substituted lower alkyl group, or OW⁴, wherein each W⁴ is independently a hydrogen atom or an optionally substituted lower alkyl group.

In some embodiments of formula (I), (IIa), (IIb), any one of (IIIa)-(IIIg), or any one of (IVa)-(IVc), above, each R^{B} is independently a fluorine atom, chlorine atom, bromine atom, methyl group, or methoxy group.

In some embodiments of formula (I), (IIa), (IIb), any one of (IIIa)-(IIIg), or any one of (IVa)-(IVc), above, U is a single bond.

In some embodiments of formula (I), (IIa), (IIb), any one of (IIIa)-(IIIg), or any one of (IVa)-(IVc), above, X is a group represented by —O-L-Y.

In some embodiments of formula (I), (IIa), (IIb), any one of (IIIa)-(IIIg), or any one of (IVa)-(IVc), above, L is a methylene group.

In some embodiments of formula (I), (IIa), (IIb), any one of (IIIa)-(IIIg), or any one of (IVa)-(IVc), above, Y is an optionally substituted benzene ring represented by formula (V)

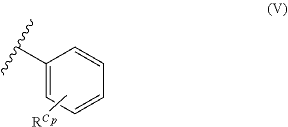

wherein each R^{C} is independently a halogen atom, an optionally substituted lower alkyl group, or OW⁹, wherein each W⁹ is independently a hydrogen atom or an optionally substituted lower alkyl group; and

p is an integer from 0 to 3.

In some embodiments of formula (I), (IIa), (IIb), any one of (IIIa)-(IIIg), any one of (IVa)-(IVc), or (V), above, Y is a substituted benzene ring represented by formula (Va)

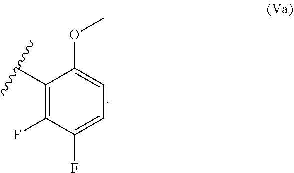 [SMILES: *c1c(OC)ccc(F)c1F]

In some embodiments, the compound presently administered to the patient is represented by formula (Ia)

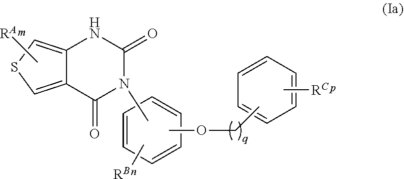

wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

q is an integer from 0 to 3;

each R^{C} is independently a halogen atom, an optionally substituted lower alkyl group, or OW⁹, wherein each W⁹ is independently a hydrogen atom or an optionally substituted lower alkyl group; and

p is an integer from 0 to 3;

or a pharmaceutically acceptable salt thereof.

In some embodiments, the compound presently administered to the patient is represented by formula (Ib)

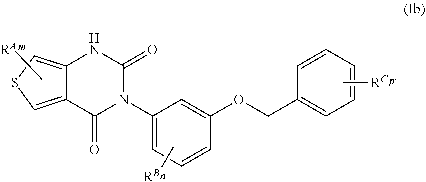

In some embodiments, the compound presently administered to the patient is represented by formula (Ic)

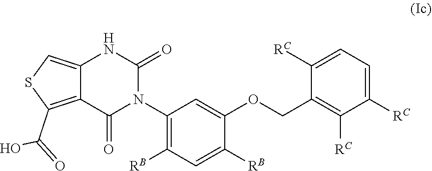

or a pharmaceutically acceptable salt thereof.

In some embodiments, the compound presently administered to the patient is 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid, represented by formula (VI)

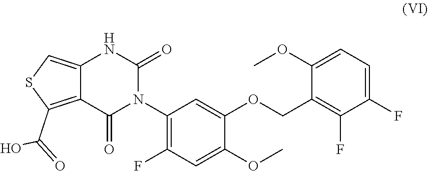 [SMILES: COc1cc(F)c(-n2c(=O)[nH]c3csc(C(=O)O)c3c2=O)cc1OCc1c(OC)ccc(F)c1F]

or a pharmaceutically acceptable salt thereof.

In some embodiments, the compound presently administered to the patient is the choline salt of the compound represented by formula (VI), choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate. It is to be understood that references herein to a compound represented by formula (VI) specifically include the choline salt of compound (VI), which is represented by formula (VIa), below.

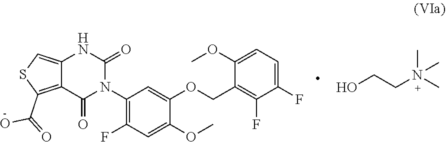

In some embodiments, the choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate is in a crystalline state.

In some embodiments, the choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate exhibits characteristic X-ray powder diffraction (XRPD) peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ.

In some embodiments, the choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm.

In some embodiments, the choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate exhibits ¹⁹F solid-state NMR peaks centered at about −151.8 ppm, −145.2 ppm, and −131.6 ppm.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is orally administered to the patient.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 25 mg to about 400 mg per dose during the treatment period (e.g., in the recited amount or in an equivalent amount of a pharmaceutically acceptable salt, such as a choline salt), such as an amount of about 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, or 400 mg of the compound of any one of formulas (I), (Ia)-(Ic), (IIa), (IIb), (IIIa)-(IIIg), (IVa)-(IVc), (V), (Va), or (VI) during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 35 mg to about 65 mg per dose during the treatment period (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per dose during the treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 50 mg per dose during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 60 mg to about 90 mg per dose during the treatment period (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per dose during the treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 75 mg per dose during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 50 mg to about 150 mg per dose during the treatment period (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per dose during the treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 100 mg per dose during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 150 mg to about 250 mg per dose during the treatment period (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per dose during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 200 mg per dose during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more doses (i.e., one or more times) per day, week, or month during the treatment period, such as from 1 to 10 times per day during the treatment period (e.g., 1 time, 2 times, 3 times, 4 times, 5 times, 6 times, 7 times, 8 times, 9 times, or 10 times per day, such as 1 time or 2 times per day), 1 to 100 times per week during the treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the treatment period, the compound of any one of formulas (I), (Ia)-(Ic), (IIa), (IIb), (IIIa)-(IIIg), (IVa)-(IVc), (V), (Va), or (VI) (e.g., (VIa)) may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more doses per day during the treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (I), (Ia)-(Ic), (IIa), (IIb), (IIIa)-(IIIg), (IVa)-(IVc), (V), (Va), or (VI) (e.g., (VIa)) may be administered to the patient in one or more unit dosage forms that collectively constitute a single dose. For example, at a given time within the treatment period, a patient may be administered a single dose of the compound of a specified amount, such as a single dose of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, or more (e.g., in the recited amount or in an equivalent amount of a pharmaceutically acceptable salt, such as a choline salt), by administration of one or more unit dosage forms of the compound to the patient. As a non-limiting example, a single dose of 200 mg of the compound may be administered to the subject by way of two individual 100-mg unit dosage forms of the compound. The two 100-mg unit dosage forms collectively constitute a single 200-mg dose of the compound if administered to the patient at substantially the same time.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 35 mg to about 65 mg per day during the treatment period (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per day during the treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 50 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 60 mg to about 90 mg per day during the treatment period (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per day during the treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 75 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 50 mg to about 150 mg per day during the treatment period (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per day during the treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 100 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 150 mg to about 250 mg per day during the treatment period (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per day during the treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 200 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in a single dose per day during the treatment period.

For example, the compound may be administered to the patient in an amount (e.g., a single dose) of from about 35 mg to about 65 mg per day during the treatment period (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per day during the treatment period (e.g., in the recited amount or in an equivalent amount of a pharmaceutically acceptable salt, such as a choline salt). In some embodiments, the compound is administered to the patient in an amount (e.g., a single dose) of about 50 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount (e.g., a single dose) of from about 60 mg to about 90 mg per day during the treatment period (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per day during the treatment period (e.g., in the recited amount or in an equivalent amount of a pharmaceutically acceptable salt, such as a choline salt). In some embodiments, the compound is administered to the patient in an amount (e.g., a single dose) of about 75 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount (e.g., a single dose) of from about 50 mg to about 150 mg per day during the treatment period (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per day during the treatment period (e.g., in the recited amount or in an equivalent amount of a pharmaceutically acceptable salt, such as a choline salt). In some embodiments, the compound is administered to the patient in an amount (e.g., a single dose) of about 100 mg per day during the treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount (e.g., a single dose) of from about 150 mg to about 250 mg per day during the treatment period (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per day during the treatment period (e.g., in the recited amount or in an equivalent amount of a pharmaceutically acceptable salt, such as a choline salt). In some embodiments, the compound is administered to the patient in an amount (e.g., a single dose) of about 200 mg per day during the treatment period (e.g., in the recited amount or in an equivalent amount of a pharmaceutically acceptable salt, such as a choline salt).

In some embodiments, the GnRH antagonist that has previously been administered to the patient (e.g., during a prior treatment period) is a compound represented by formula (VII)

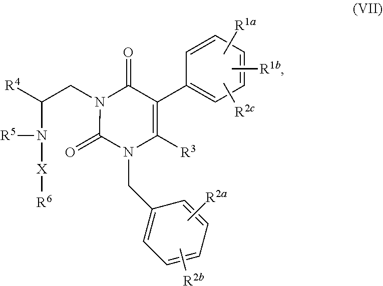

wherein R₁ₐ, R_{1b} and R_{1c} are the same or different and are each independently hydrogen, halogen, C₁₋₄alkyl, hydroxy or alkoxy, or R₁ₐ and R_{1b} taken together form —OCH₂O— or —OCH₂CH₂—;

R₂ₐ and R_{2b} are the same or different and are each independently hydrogen, halogen, trifluoromethyl, cyano or —SO₂CH₃;

R₃ is hydrogen or methyl;

R₄ is phenyl or C₃₋₇alkyl;

R₅ is hydrogen or C₁₋₄alkyl;

R₆ is —COOH or an acid isostere; and

X is C₁₋₆alkanediyl optionally substituted with from 1 to 3 C₁₋₆alkyl groups;

or a pharmaceutically acceptable salt thereof.

In some embodiments, the GnRH antagonist that has previously been administered to the patient (e.g., during the prior treatment period) is a compound represented by formula (VIII)

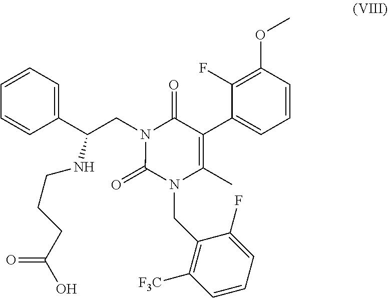 [SMILES: COc1cccc(-c2c(C)n(Cc3c(F)cccc3C(F)(F)F)c(=O)n(C[C@H](NCCCC(=O)O)c3ccccc3)c2=O)c1F]

or a pharmaceutically acceptable salt thereof. In some embodiments, the GnRH antagonist that has previously been administered to the patient (e.g., during the prior treatment period) is the sodium salt of the compound represented by formula (VIII), which is represented by formula (IX), below.

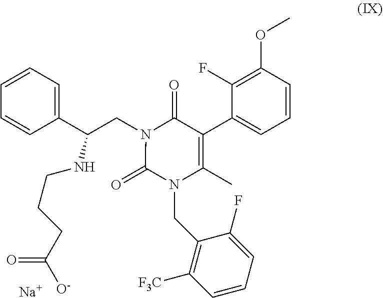 [SMILES: COc1cccc(-c2c(C)n(Cc3c(F)cccc3C(F)(F)F)c(=O)n(C[C@H](NCCCC(=O)[O-])c3ccccc3)c2=O)c1F ; [Na+]]

In some embodiments, the compound of any one of formulas (VII)-(IX) has previously been administered to the patient in an amount of from about 50 mg to about 650 mg per dose during the prior treatment period (e.g., in the recited amount or in an equivalent amount of a pharmaceutically acceptable salt, such as a sodium salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, or 650 mg per dose during the prior treatment period (e.g., in the recited amount or in an equivalent amount of a pharmaceutically acceptable salt, such as a sodium salt). In some embodiments, the compound of any one of formulas (VII)-(IX) has previously been administered to the patient in an amount of about 150 mg per dose during the prior treatment period, 300 mg per dose during the prior treatment period, 400 mg per dose during the prior treatment period, or 600 mg per dose during the prior treatment period (e.g., in the recited amount or in an equivalent amount of a pharmaceutically acceptable salt, such as a sodium salt).

In some embodiments, the compound of any one of formulas (VII)-(IX) has previously been administered to the patient in one or more doses (i.e., one or more times) per day, week, or month during the prior treatment period, such as from 1 to 10 times per day during the prior treatment period (e.g., 1 time, 2 times, 3 times, 4 times, 5 times, 6 times, 7 times, 8 times, 9 times, or 10 times per day, such as 1 time or 2 times per day), 1 to 100 times per week during the prior treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the prior treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the prior treatment period, the compound of any one of formulas (VII)-(IX) may have been administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (VII)-(IX) may have been administered to the patient in one or more doses per day during the prior treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (VII)-(IX) may have been administered to the patient in one or more unit dosage forms that collectively constitute a single dose. For example, a patient may have been administered a single dose of the compound of a specified amount, such as a single dose of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, or more (e.g., in the recited amount or in an equivalent amount of a pharmaceutically acceptable salt, such as a sodium salt), by administration of one or more unit dosage forms of the compound to the patient. As a non-limiting example, a single dose of 300 mg of the compound may have been administered to the subject by way of two individual 150-mg unit dosage forms of the compound. The two 150-mg unit dosage forms collectively constitute a single 300-mg dose of the compound if administered to the patient at substantially the same time.

In some embodiments, the compound of any one of formulas (VII)-(IX) has previously been administered to the patient in one or more daily doses (e.g., from 1 to 10 doses, such as 1, 2, 3, 4, 5, 6, 7, 8, 9, or 10 doses, or more) totaling from about 50 mg to about 650 mg per day during the prior treatment period (e.g., in the recited amount or in an equivalent amount of a pharmaceutically acceptable salt, such as a sodium salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, or 650 mg per day during the prior treatment period (e.g., in the recited amount or in an equivalent amount of a pharmaceutically acceptable salt, such as a sodium salt). In some embodiments, the compound has previously been administered to the patient in one or more daily doses (e.g., from 1 to 10 doses, such as 1, 2, 3, 4, 5, 6, 7, 8, 9, or 10 doses, or more) totaling an amount of about 150 mg per day during the prior treatment period, 300 mg per day during the prior treatment period, 400 mg per day during the prior treatment period (e.g., 200 mg administered twice daily), or 600 mg per day during the prior treatment period (e.g., 300 mg administered twice daily) (e.g., in the recited amount or in an equivalent amount of a pharmaceutically acceptable salt, such as a sodium salt).

In some embodiments, the GnRH antagonist that has previously been administered to the patient (e.g., during the prior treatment period) is a compound represented by formula (X)

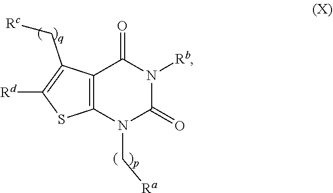

wherein R^{a} is a hydrogen atom, an optionally substituted aryl group (such as an aryl group that may have 1 to 5 substituents selected from halogen, nitro, cyano, amino, a carboxyl group that may be esterified or amidated, an alkylenedioxy, alkyl, alkoxy, alkylthio, alkylsulfinyl, and alkylsulfonyl), an optionally substituted cycloalkyl group, or an optionally substituted heterocyclic group;

R^{b} is an optionally substituted nitrogen-containing heterocyclic group;

R^{c} is an optionally substituted amino group;

R^{d} is an optionally substituted aryl group;
p is an integer from 0 to 3; and

q is an integer from 0 to 3;

or a pharmaceutically acceptable salt thereof.

In some embodiments, the GnRH antagonist that has previously been administered to the patient (e.g., during the prior treatment period) is a compound represented by formula (XI)

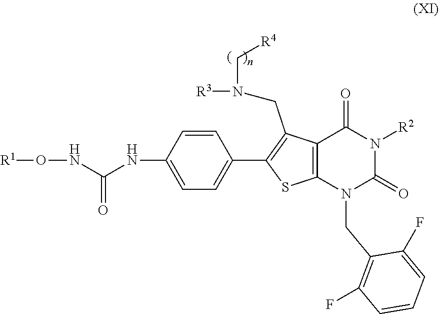

wherein R¹ is C₁₋₄alkyl;

R² is (1) a C₁₋₆alkyl which may have a substituent selected from the group consisting of (1′) a hydroxy group, (2′) a C₁₋₄alkoxy, (3′) a C₁₋₄alkoxy-carbonyl, (4′) a di-C₁₋₄alkyl-carbamoyl, (5′) a 5- to 7-membered nitrogen-containing heterocyclic group, (6′) a C₁₋₄alkyl-carbonyl and (7′) a halogen, (2) a C₃₋₈ cycloalkyl which may have (1′) a hydroxy group or (2′) a mono-C₁₋₄alkyl-carbonylamino, (3) a 5- to 7-membered nitrogen-containing heterocyclic group which may have a substituent selected from the group consisting of (1′) a halogen, (2′) a hydroxy group, (3′) a C₁₋₄alkyl and (4′) a C₁₋₄alkoxy, (4) a phenyl which may have a substituent selected from the group consisting of (1′) a halogen, (2′) a C₁₋₄alkoxy-C₁₋₄alkyl, (3′) a mono-C₁₋₄alkyl-carbamoyl-C₁₋₄alkyl, (4′) a C₁₋₄alkoxy and (5′) a mono-C₁₋₄alkylcarbamoyl-C₁₋₄alkoxy, or (5) a C₁₋₄alkoxy;

R³ is C₁₋₄alkyl;

R⁴ is (1) hydrogen, (2) C₁₋₄alkoxy, (3) C₆₋₁₀aryl, (4) N—C₁₋₄alkyl-N—C₁₋₄alkylsulfonylamino, (5) hydroxyl, or (6) a 5- to 7-membered nitrogen-containing heterocyclic group which may have a substituent selected from the group consisting of (1′) oxo, (2′) a C₁₋₄alkyl, (3′) a hydroxy-C₁₋₄alkyl, (4′) a C₁₋₄alkoxy-carbonyl, (5′) a mono-C₁₋₄ alkyl-carbamoyl and (6′) a C₁₋₄ alkylsulfonyl; and

n is an integer from 1 to 4;

optionally provided that when R² is a phenyl which may have a substituent, R⁴ is a 5- to 7-membered nitrogen-containing heterocyclic group which may have a substituent selected from the group consisting of (1) oxo, (2) hydroxy-C₁₋₄alkyl, (3) C₁₋₄alkoxy-carbonyl, (4) mono-C₁₋₄alkyl-carbamoyl and (5) C₁₋₄alkylsulfonyl;

or a pharmaceutically acceptable salt thereof. In some embodiments, the GnRH antagonist that has previously been administered to the patient (e.g., during the prior treatment period) is a compound represented by formula (XII), below.

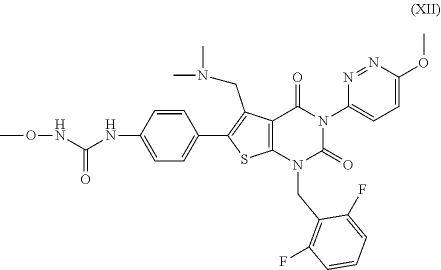

In some embodiments, the compound of any one of formulas (X)-(XII) has previously been administered to the patient in an amount of from about 10 mg to about 60 mg per dose during the prior treatment period (e.g., in the recited amount or in an equivalent amount of a pharmaceutically acceptable salt, such as a chlorine salt), such as an amount of about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg per dose during the prior treatment period (e.g., in the recited amount or in an equivalent amount of a pharmaceutically acceptable salt, such as a chlorine salt). In some embodiments, the compound of any one of formulas (X)-(XII) has previously been administered to the patient in an amount of about 40 mg per dose during the prior treatment period (e.g., in the recited amount or in an equivalent amount of a pharmaceutically acceptable salt, such as a chlorine salt).

In some embodiments, the compound of any one of formulas (X)-(XII) has previously been administered to the patient in one or more doses (i.e., one or more times) per day, week, or month during the prior treatment period, such as from 1 to 10 times per day during the prior treatment period (e.g., 1 time, 2 times, 3 times, 4 times, 5 times, 6 times, 7 times, 8 times, 9 times, or 10 times per day, such as 1 time or 2 times per day), 1 to 100 times per week during the prior treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the prior treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the prior treatment period, the compound of any one of formulas (X)-(XII) may have been administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (X)-(XII) has been administered to the patient in one or more doses per day during the prior treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (I)-(XII) may have been administered to the patient in one or more unit dosage forms that collectively constitute a single dose. For example, a patient may have been administered a single dose of the compound of a specified amount, such as a single dose of 25 mg, 50 mg, 75 mg, 100 mg, or more (e.g., in the recited amount or in an equivalent amount of a pharmaceutically acceptable salt, such as a chlorine salt), by administration of one or more unit dosage forms of the compound to the patient. As a non-limiting example, a single dose of 40 mg of the compound may have been administered to the subject by way of two individual 20-mg unit dosage forms of the compound. The two 20-mg unit dosage forms collectively constitute a single 40-mg dose of the compound if administered to the patient at substantially the same time.

In some embodiments, the compound of any one of formulas (X)-(XII) has previously been administered to the patient in one or more daily doses (e.g., from 1 to 10 doses, such as 1, 2, 3, 4, 5, 6, 7, 8, 9, or 10 doses, or more) totaling from about 10 mg to about 60 mg per day during the prior treatment period (e.g., in the recited amount or in an equivalent amount of a pharmaceutically acceptable salt, such as a chlorine salt), such as an amount of about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg per day during the prior treatment period (e.g., in the recited amount or in an equivalent amount of a pharmaceutically acceptable salt, such as a chlorine salt). In some embodiments, the compound has been administered to the patient in one or more daily doses (e.g., from 1 to 10 doses, such as 1, 2, 3, 4, 5, 6, 7, 8, 9, or 10 doses, or more) totaling from about 20 mg to about 50 mg per day during the prior treatment period (e.g., in the recited amount or in an equivalent amount of a pharmaceutically acceptable salt, such as a chlorine salt), such as an amount of about 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg per day during the prior treatment period (e.g., in the recited amount or in an equivalent amount of a pharmaceutically acceptable salt, such as a chlorine salt). In some embodiments, the compound has been administered to the patient in one or more daily doses (e.g., from 1 to 10 doses, such as 1, 2, 3, 4, 5, 6, 7, 8, 9, or 10 doses, or more) totaling an amount of about 40 mg per day during the prior treatment period (e.g., a single daily dose of 40 mg) (e.g., in the recited amount or in an equivalent amount of a pharmaceutically acceptable salt, such as a chlorine salt).

In some embodiments, the GnRH antagonist that has previously been administered to the patient (e.g., during the prior treatment period) is a compound represented by formula (XIII)

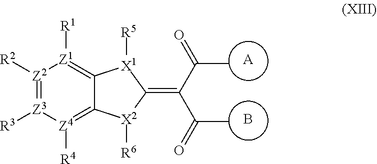

wherein R¹, R², R³ and R⁴ are the same or different, and are each independently selected from hydrogen, nitro, cyano, halogen, an optionally substituted hydrocarbon group, an optionally substituted heterocyclic group, hydroxy, alkoxy, carboxy, optionally substituted acyl-O—, optionally substituted acyl, a substituent —S(O)n₁₀₁- (wherein n₁₀₁ is an integer of 0 to 2), H—S(O)n₁₀₁-, optionally substituted carbamoyl, optionally substituted sulfamoyl, optionally substituted amino, and two adjacent groups selected from the group of R¹, R², R³ and R⁴ may combine to form an aryl or a carbocyclic (e.g., cycloalkenyl) group;

R⁵ and R⁶ are the same or different and are each independently selected from hydrogen, halogen, optionally substituted hydrocarbon, and optionally substituted amino,

X¹ and X² are the same or different and are each independently selected from N, S and O;

A and B are the same or different and are each independently selected from optionally substituted aryl and optionally substituted heterocyclyl, and

Z¹, Z², Z³ and Z⁴ are each independently selected from C and N; optionally provided that 1) when X¹ and X² each is S or O, one or both of the corresponding R⁵ and R⁶ are absent; and/or 2) when one to four of Z¹, Z², Z³ and/or Z⁴ are N, the corresponding R¹, R², R³ and/or R⁴ are absent;

or a pharmaceutically acceptable salt thereof. In some embodiments, the GnRH antagonist that has previously been administered to the patient (e.g., during the prior treatment period) is a compound represented by formula (XIV), below.

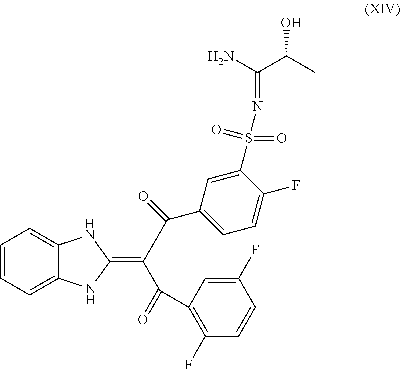 [SMILES: C[C@@H](O)/C(N)=N/S(=O)(=O)c1cc(C(=O)C(C(=O)c2cc(F)ccc2F)=C2Nc3ccccc3N2)ccc1F]

In some embodiments, the compound of any one of formulas (XIII) and (XIV), or SKI2670 or BAY-784, has previously been administered to the patient in one or more doses (i.e., one or more times) per day, week, or month during the prior treatment period, such as from 1 to 10 times per day during the prior treatment period (e.g., 1 time, 2 times, 3 times, 4 times, 5 times, 6 times, 7 times, 8 times, 9 times, or 10 times per day, such as 1 time or 2 times per day), 1 to 100 times per week during the prior treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the prior treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the prior treatment period, the compound of any one of formulas (XIII) and (XIV), or SKI2670 or BAY-784, may have been administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (XIII) and (XIV), or SKI2670 or BAY-784, has been administered to the patient in one or more doses per day during the prior treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

In some embodiments of the disclosure, add-back therapy is administered (e.g., periodically administered) to the patient during the treatment period.

In some embodiments, the add-back therapy is administered to the patient concurrently with the GnRH antagonist, prior to administration of the GnRH antagonist, or following administration of the GnRH antagonist. In some embodiments, add-back therapy is administered as a fixed dose combination containing a GnRH antagonist, estrogen, and one or more additional agents, such as a progestin, in a single pharmaceutical composition. For instance, add-back therapy may be administered as a fixed dose combination of a GnRH antagonist, estrogen (e.g., in the form of β17-estradiol, ethinyl estradiol, or a conjugated estrogen, such as a conjugated equine estrogen) and/or a progestin (e.g., norethindrone or a compound that is metabolized in vivo to produce norethindrone, such as an ester of norethindrone that is de-esterified in vivo to produce norethindrone, for instance, norethindrone acetate (also referred to herein as “NETA”), among other agents, such as progesterone, norgestimate, medroxyprogesterone, and drospirenone) in the form of a single pharmaceutical composition, such as a single tablet, capsule, gel cap, powder, liquid solution, or liquid suspension. In some embodiments, the add-back therapy is administered orally, transdermally, or intravaginally.

In some embodiments, the add-back therapy is administered to the patient in one or more doses per day, week, month, or year, such as daily, for example, from 1 to 10 times daily, or more (e.g., 1, 2, 3, 4, 5, 6, 7, 8, 9, 10, or more, times daily). In some embodiments, the add-back therapy is administered to the patient once daily, for example, concurrently with the GnRH antagonist. For example, the GnRH antagonist may be administered to the patient orally, and concurrently with oral administration of the GnRH antagonist, the add-back therapy may be administered to the patient orally, transdermally, or intravaginally. In some embodiments, the add-back therapy is administered to the patient in the form of a pharmaceutical composition that further includes the GnRH antagonist, such as a single tablet, capsule, gel cap, powder, liquid solution, or liquid suspension, for instance, as described above and herein.

In some embodiments, the add-back therapy is administered to the patient once daily, following administration of the GnRH antagonist. For example, the GnRH antagonist may be administered to the patient orally, and following oral administration of the GnRH antagonist, the add-back therapy may be administered to the patient orally, transdermally, or intravaginally.

In some embodiments, the add-back therapy is administered to the patient once daily, prior to administration of the GnRH antagonist. For example, the GnRH antagonist may be administered to the patient orally, and prior to oral administration of the GnRH antagonist, the add-back therapy may be administered to the patient orally, transdermally, or intravaginally.

In some embodiments, the add-back therapy includes an estrogen. In some embodiments, the estrogen is selected from the group consisting of β17-estradiol, ethinyl estradiol, and conjugated estrogens, such as conjugated equine estrogens.

In some embodiments, the estrogen is β17-estradiol. The β17-estradiol may be administered to the patient, for example, at a dose of from about 0.1 mg to about 2.5 mg, such as at a dose of about 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, or 2.5 mg, for instance, by oral administration. In some embodiments, the β17-estradiol is administered to the patient at a dose of 1.0 mg (e.g., once daily), for instance, by oral administration. In some embodiments, the β17-estradiol is administered to the patient at a dose of 0.5 mg (e.g., once daily), for instance, by oral administration.

The β17-estradiol may be administered to the patient one or more times per day, week, or month. The β17-estradiol may be administered to the patient, for example, in an amount of from about 0.1 mg/day to about 2.5 mg/day, such as in an amount of about 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, 2.0 mg/day, 2.1 mg/day, 2.2 mg/day, 2.3 mg/day, 2.4 mg/day, or 2.5 mg/day, for instance, by oral administration. In some embodiments, the β17-estradiol is administered to the patient in an amount of 1.0 mg/day, for instance, by oral administration. In some embodiments, the β17-estradiol is administered to the patient in an amount of 0.5 mg/day, for instance, by oral administration.

In some embodiments, the estrogen is ethinyl estradiol. The ethinyl estradiol may be administered to the patient, for example, at a dose of from about 1.0 μg to about 6.0 μg, such as at a dose of about 1.0 μg, 1.1 μg, 1.2 μg, 1.3 μg, 1.4 μg, 1.5 μg, 1.6 μg, 1.7 μg, 1.8 μg, 1.9 μg, 2.0 μg, 2.1 μg, 2.2 μg, 2.3 μg, 2.4 μg, 2.5 μg, 2.6 μg, 2.7 μg, 2.8 μg, 2.9 μg, 3.0 μg, 3.1 μg, 3.2 μg, 3.3 μg, 3.4 μg, 3.5 μg, 3.6 μg, 3.7 μg, 3.8 μg, 3.9 μg, 4.0 μg, 4.1 μg, 4.2 μg, 4.2 μg, 4.3 μg, 4.4 μg, 4.5 μg, 4.6 μg, 4.7 μg, 4.8 μg, 4.9 μg, 5.0 μg, 5.1 μg, 5.2 μg, 5.3 μg, 5.4 μg, 5.5 μg, 5.6 μg, 5.7 μg, 5.8 μg, 5.9 μg, or 6.0 μg, for instance, by oral administration. In some embodiments, the ethinyl estradiol is administered to the patient at a dose of 5.0 μg, for instance, by oral administration. In some embodiments, the ethinyl estradiol is administered to the patient at a dose of 2.5 μg, for instance, by oral administration.

The ethinyl estradiol may be administered to the patient one or more times per day, week, or month. The ethinyl estradiol may be administered to the patient, for example, in an amount of about 1.0 μg/day to about 6.0 μg/day, such as in an amount of about 1.0 μg/day, 1.1 μg/day, 1.2 μg/day, 1.3 μg/day, 1.4 μg/day, 1.5 μg/day, 1.6 μg/day, 1.7 μg/day, 1.8 μg/day, 1.9 μg/day, 2.0 μg/day, 2.1 μg/day, 2.2 μg/day, 2.3 μg/day, 2.4 μg/day, 2.5 μg/day, 2.6 μg/day, 2.7 μg/day, 2.8 μg/day, 2.9 μg/day, 3.0 μg/day, 3.1 μg/day, 3.2 μg/day, 3.3 μg/day, 3.4 μg/day, 3.5 μg/day, 3.6 μg/day, 3.7 μg/day, 3.8 μg/day, 3.9 μg/day, 4.0 μg/day, 4.1 μg/day, 4.2 μg/day, 4.2 μg/day, 4.3 μg/day, 4.4 μg/day, 4.5 μg/day, 4.6 μg/day, 4.7 μg/day, 4.8 μg/day, 4.9 μg/day, 5.0 μg/day, 5.1 μg/day, 5.2 μg/day, 5.3 μg/day, 5.4 μg/day, 5.5 μg/day, 5.6 μg/day, 5.7 μg/day, 5.8 μg/day, 5.9 μg/day, or 6.0 μg/day, for instance, by oral administration. In some embodiments, the ethinyl estradiol is administered to the patient in an amount of 5.0 μg/day, for instance, by oral administration. In some embodiments, the ethinyl estradiol is administered to the patient in an amount of 2.5 μg/day, for instance, by oral administration.

In some embodiments, the estrogen is a conjugated estrogen, such as a conjugated equine estrogen. The conjugated estrogen may be administered to the patient, for example, at a dose of from about 0.1 mg to about 2.0 mg, such as at a dose of about 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, or 2.0 mg, for instance, by oral administration. In some embodiments, the conjugated estrogen is administered to the patient at a dose of 0.625 mg, for instance, by oral administration. In some embodiments, the conjugated estrogen is administered to the patient at a dose of 0.45 mg, for instance, by oral administration. In some embodiments, the conjugated estrogen is administered to the patient at a dose of 0.3 mg, for instance, by oral administration.

The conjugated estrogen may be administered to the patient one or more times per day, week, or month. The conjugated estrogen may be administered to the patient, for example, in an amount of from about 0.1 mg/day to about 2.0 mg/day, such as in an amount of about 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, or 2.0 mg/day, for instance, by oral administration. In some embodiments, the conjugated estrogen is administered to the patient in an amount of 0.625 mg/day, for instance, by oral administration. In some embodiments, the conjugated estrogen is administered to the patient in an amount of 0.45 mg/day, for instance, by oral administration. In some embodiments, the conjugated estrogen is administered to the patient in an amount of 0.3 mg/day, for instance, by oral administration. In some embodiments, the add-back therapy includes a progestin. In some embodiments, the progestin is selected from the group consisting of norethindrone or an ester thereof, such as norethindrone acetate, or another agent such as progesterone, norgestimate, medroxyprogesterone, or drospirenone.

In some embodiments, the progestin is norethindrone or a compound that is metabolized in vivo to produce norethindrone, such as an ester of norethindrone that is de-esterified in vivo to produce norethindrone, for instance, norethindrone acetate.

In some embodiments, the progestin is norethindrone. The norethindrone may be administered to the patient, for example, at a dose of from about 0.05 mg to about 5.0 mg, such as at a dose of about 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, or 5.0 mg, for instance, by oral administration. In some embodiments, the norethindrone is administered to the patient at a dose of 1.0 mg, for instance, by oral administration. In some embodiments, the norethindrone is administered to the patient at a dose of 0.5 mg, for instance, by oral administration. In some embodiments, the norethindrone is administered to the patient at a dose of 0.1 mg, for instance, by oral administration.

The norethindrone may be administered to the patient one or more times per day, week, or month. The norethindrone may be administered to the patient, for example, in an amount of from about 0.05 mg/day to about 5.0 mg/day, such as in an amount of about 0.05 mg/day, 0.06 mg/day, 0.07 mg/day, 0.08 mg/day, 0.09 mg/day, 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, 2.0 mg/day, 2.1 mg/day, 2.2 mg/day, 2.3 mg/day, 2.4 mg/day, 2.5 mg/day, 2.6 mg/day, 2.7 mg/day, 2.8 mg/day, 2.9 mg/day, 3.0 mg/day, 3.1 mg/day, 3.2 mg/day, 3.3 mg/day, 3.4 mg/day, 3.5 mg/day, 3.6 mg/day, 3.7 mg/day, 3.8 mg/day, 3.9 mg/day, 4.0 mg/day, 4.1 mg/day, 4.2 mg/day, 4.3 mg/day, 4.4 mg/day, 4.5 mg/day, 4.6 mg/day, 4.7 mg/day, 4.8 mg/day, 4.9 mg/day, or 5.0 mg/day, for instance, by oral administration. In some embodiments, the norethindrone is administered to the patient in an amount of 1.0 mg/day, for instance, by oral administration. In some embodiments, the norethindrone is administered to the patient in an amount of 0.5 mg/day, for instance, by oral administration. In some embodiments, the norethindrone is administered to the patient in an amount of 0.1 mg/day, for instance, by oral administration.

In some embodiments, the progestin is norethindrone acetate. The norethindrone acetate may be administered to the patient, for example, at a dose of from about 0.05 mg to about 5.0 mg, such as at a dose of about 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, or 5.0 mg, for instance, by oral administration. In some embodiments, the norethindrone acetate is administered to the patient at a dose of 1.0 mg (e.g., once daily), for instance, by oral administration. In some embodiments, the norethindrone acetate is administered to the patient at a dose of 0.5 mg (e.g., once daily), for instance, by oral administration. In some embodiments, the norethindrone acetate is administered to the patient at a dose of 0.1 mg (e.g., once daily), for instance, by oral administration.

The norethindrone acetate may be administered to the patient one or more times per day, week, or month. The norethindrone acetate may be administered to the patient, for example, in an amount of from about 0.05 mg/day to about 5.0 mg/day, such as in an amount of about 0.05 mg/day, 0.06 mg/day, 0.07 mg/day, 0.08 mg/day, 0.09 mg/day, 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, 2.0 mg/day, 2.1 mg/day, 2.2 mg/day, 2.3 mg/day, 2.4 mg/day, 2.5 mg/day, 2.6 mg/day, 2.7 mg/day, 2.8 mg/day, 2.9 mg/day, 3.0 mg/day, 3.1 mg/day, 3.2 mg/day, 3.3 mg/day, 3.4 mg/day, 3.5 mg/day, 3.6 mg/day, 3.7 mg/day, 3.8 mg/day, 3.9 mg/day, 4.0 mg/day, 4.1 mg/day, 4.2 mg/day, 4.3 mg/day, 4.4 mg/day, 4.5 mg/day, 4.6 mg/day, 4.7 mg/day, 4.8 mg/day, 4.9 mg/day, or 5.0 mg/day, for instance, by oral administration. In some embodiments, the norethindrone acetate is administered to the patient in an amount of 1.0 mg/day, for instance, by oral administration. In some embodiments, the norethindrone acetate is administered to the patient in an amount of 0.5 mg/day, for instance, by oral administration. In some embodiments, the norethindrone acetate is administered to the patient in an amount of 0.1 mg/day, for instance, by oral administration.

In some embodiments, the progestin is progesterone. The progesterone may be administered to the patient, for example, at a dose of from about 50 mg to about 250 mg, such as a dose of about 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, or 250 mg, for instance, by oral administration. In some embodiments, the progesterone is administered to the patient at a dose of 200 mg, for instance, by oral administration. In some embodiments, the progesterone is administered to the patient at a dose of 100 mg, for instance, by oral administration.

The progesterone may be administered to the patient one or more times per day, week, or month. The progesterone may be administered to the patient, for example, in an amount of from about 50 mg/day to about 250 mg/day, such as a dose of about 50 mg/day, 55 mg/day, 60 mg/day, 65 mg/day, 70 mg/day, 75 mg/day, 80 mg/day, 85 mg/day, 90 mg/day, 95 mg/day, 100 mg/day, 105 mg/day, 110 mg/day, 115 mg/day, 120 mg/day, 125 mg/day, 130 mg/day, 135 mg/day, 140 mg/day, 145 mg/day, 150 mg/day, 155 mg/day, 160 mg/day, 165 mg/day, 170 mg/day, 175 mg/day, 180 mg/day, 185 mg/day, 190 mg/day, 195 mg/day, 200 mg/day, 205 mg/day, 210 mg/day, 215 mg/day, 220 mg/day, 225 mg/day, 230 mg/day, 235 mg/day, 240 mg/day, 245 mg/day, or 250 mg/day, for instance, by oral administration. In some embodiments, the progesterone is administered to the patient in an amount of 200 mg/day, for instance, by oral administration. In some embodiments, the progesterone is administered to the patient in an amount of 100 mg/day, for instance, by oral administration.

In some embodiments, the progestin is norgestimate. The norgestimate may be administered to the patient, for example, at a dose of from about 0.01 mg to about 2.0 mg, such as at a dose of about 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, or 2.0 mg, for instance, by oral administration. In some embodiments, the norgestimate is administered to the patient at a dose of 0.09 mg, for instance, by oral administration.

The norgestimate may be administered to the patient one or more times per day, week, or month. The norgestimate may be administered to the patient, for example, in an amount of from about 0.01 mg/day to about 2.0 mg/day, such as in an amount of about 0.01 mg/day, 0.02 mg/day, 0.03 mg/day, 0.04 mg/day, 0.05 mg/day, 0.06 mg/day, 0.07 mg/day, 0.08 mg/day, 0.09 mg/day, 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, or 2.0 mg/day, for instance, by oral administration. In some embodiments, the norgestimate is administered to the patient in an amount of 0.09 mg/day, for instance, by oral administration.

In some embodiments, the progestin is medroxyprogesterone. The medroxyprogesterone may be administered to the patient, for example, at a dose of from about 0.5 mg to about 10.0 mg, such as at a dose of about 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, 8.0 mg, 8.1 mg, 8.2 mg, 8.3 mg, 8.4 mg, 8.5 mg, 8.6 mg, 8.7 mg, 8.8 mg, 8.9 mg, 9.0 mg, 9.1 mg, 9.2 mg, 9.3 mg, 9.4 mg, 9.5 mg, 9.6 mg, 9.7 mg, 9.8 mg, 9.9 mg, or 10.0 mg, for instance, by oral administration. In some embodiments, the medroxyprogesterone is administered to the patient at a dose of 5.0 mg, for instance, by oral administration. In some embodiments, the medroxyprogesterone is administered to the patient at a dose of 2.5 mg, for instance, by oral administration. In some embodiments, the medroxyprogesterone is administered to the patient at a dose of 1.5 mg, for instance, by oral administration.

The medroxyprogesterone may be administered to the patient one or more times per day, week, or month. The medroxyprogesterone may be administered to the patient, for example, in an amount of from about 0.5 mg/day to about 10.0 mg/day, such as in an amount of about 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, 2.0 mg/day, 2.1 mg/day, 2.2 mg/day, 2.3 mg/day, 2.4 mg/day, 2.5 mg/day, 2.6 mg/day, 2.7 mg/day, 2.8 mg/day, 2.9 mg/day, 3.0 mg/day, 3.1 mg/day, 3.2 mg/day, 3.3 mg/day, 3.4 mg/day, 3.5 mg/day, 3.6 mg/day, 3.7 mg/day, 3.8 mg/day, 3.9 mg/day, 4.0 mg/day, 4.1 mg/day, 4.2 mg/day, 4.3 mg/day, 4.4 mg/day, 4.5 mg/day, 4.6 mg/day, 4.7 mg/day, 4.8 mg/day, 4.9 mg/day, 5.0 mg/day, 5.1 mg/day, 5.2 mg/day, 5.3 mg/day, 5.4 mg/day, 5.5 mg/day, 5.6 mg/day, 5.7 mg/day, 5.8 mg/day, 5.9 mg/day, 6.0 mg/day, 6.1 mg/day, 6.2 mg/day, 6.3 mg/day, 6.4 mg/day, 6.5 mg/day, 6.6 mg/day, 6.7 mg/day, 6.8 mg/day, 6.9 mg/day, 7.0 mg/day, 7.1 mg/day, 7.2 mg/day, 7.3 mg/day, 7.4 mg/day, 7.5 mg/day, 7.6 mg/day, 7.7 mg/day, 7.8 mg/day, 7.9 mg/day, 8.0 mg/day, 8.1 mg/day, 8.2 mg/day, 8.3 mg/day, 8.4 mg/day, 8.5 mg/day, 8.6 mg/day, 8.7 mg/day, 8.8 mg/day, 8.9 mg/day, 9.0 mg/day, 9.1 mg/day, 9.2 mg/day, 9.3 mg/day, 9.4 mg/day, 9.5 mg/day, 9.6 mg/day, 9.7 mg/day, 9.8 mg/day, 9.9 mg/day, or 10.0 mg/day, for instance, by oral administration. In some embodiments, the medroxyprogesterone is administered to the patient in an amount of 5.0 mg/day, for instance, by oral administration. In some embodiments, the medroxyprogesterone is administered to the patient in an amount of 2.5 mg/day, for instance, by oral administration. In some embodiments, the medroxyprogesterone is administered to the patient in an amount of 1.5 mg/day, for instance, by oral administration.

In some embodiments, the progestin is drospirenone. The drospirenone may be administered to the patient, for example, at a dose of from about 0.1 mg to about 1.0 mg, such as at a dose of about 0.1 mg, 0.2 mg, 0.3 mg, 0.4 mg, 0.5 mg, 0.6 mg, 0.7 mg, 0.8 mg, 0.9 mg, or 1.0 mg, for instance, by oral administration. In some embodiments, the drospirenone is administered to the patient at a dose of 0.5 mg, for instance, by oral administration. In some embodiments, the drospirenone is administered to the patient at a dose of 0.25 mg, for instance, by oral administration.

The drospirenone may be administered to the patient one or more times per day, week, or month. The drospirenone may be administered to the patient, for example, in an amount of from about 0.1 mg/day to about 1.0 mg/day, such as in an amount of about 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, or 1.0 mg/day, for instance, by oral administration. In some embodiments, the drospirenone is administered to the patient in an amount of 0.5 mg/day, for instance, by oral administration. In some embodiments, the drospirenone is administered to the patient in an amount of 0.25 mg/day, for instance, by oral administration.

In some embodiments, the add-back therapy includes an estrogen and a progestin. In some embodiments, the add-back therapy includes β17-estradiol and norethindrone or a compound that is metabolized in vivo to produce norethindrone, such as an ester of norethindrone that is de-esterified in vivo to produce norethindrone, for instance, norethindrone acetate.

In some embodiments, the add-back therapy includes from about 0.75 mg to about 1.25 mg of β17-estradiol, e.g., administered orally, and from about 0.25 mg to about 0.75 mg of norethindrone or a compound that is metabolized in vivo to produce norethindrone, such as an ester of norethindrone that is de-esterified in vivo to produce norethindrone, for instance, norethindrone acetate, e.g., administered orally. In some embodiments, the add-back therapy includes 1.0 mg of β17-estradiol, e.g., administered orally, and 0.5 mg of norethindrone or a compound that is metabolized in vivo to produce norethindrone, such as an ester of norethindrone that is de-esterified in vivo to produce norethindrone, for instance, norethindrone acetate, e.g., administered orally. In some embodiments, the add-back therapy includes 1.0 mg of β17-estradiol, e.g., administered orally, and, in the same pharmaceutical composition, 0.5 mg of norethindrone or a compound that is metabolized in vivo to produce norethindrone, such as an ester of norethindrone that is de-esterified in vivo to produce norethindrone, for instance, norethindrone acetate, e.g., administered orally. In some embodiments, the add-back therapy includes 1.0 mg of β17-estradiol, e.g., administered orally, and, in a separate pharmaceutical composition, 0.5 mg of norethindrone or a compound that is metabolized in vivo to produce norethindrone, such as an ester of norethindrone that is de-esterified in vivo to produce norethindrone, for instance, norethindrone acetate, e.g., administered orally.

In some embodiments, the GnRH antagonist is administered to the patient in a fixed-dose composition that contains the GnRH antagonist (e.g., in an amount of about 100 mg or about 200 mg of the compound of any one of formulas (I)-(VI)), from about 0.75 mg to about 1.25 mg of β17-estradiol, and from about 0.25 mg to about 0.75 mg of norethindrone or a compound that is metabolized in vivo to produce norethindrone, such as an ester of norethindrone that is de-esterified in vivo to produce norethindrone, for instance, norethindrone acetate. In some embodiments, the GnRH antagonist is administered to the patient in a fixed-dose composition that contains the GnRH antagonist (e.g., in an amount of about 100 mg or about 200 mg of the compound of any one of formulas (I)-(VI)), about 1.0 mg of β17-estradiol (e.g., 1.0 mg of β17-estradiol), and about 0.5 mg of norethindrone or a compound that is metabolized in vivo to produce norethindrone, such as an ester of norethindrone that is de-esterified in vivo to produce norethindrone, for instance, norethindrone acetate (e.g., 0.5 mg of norethindrone or a compound that is metabolized in vivo to produce norethindrone, such as an ester of norethindrone that is de-esterified in vivo to produce norethindrone, for instance, norethindrone acetate). In some embodiments, the GnRH antagonist is administered to the patient in a fixed-dose composition that contains the GnRH antagonist (e.g., in an amount of about 100 mg or about 200 mg of the compound of any one of formulas (I)-(VI)), 1.0 mg of β17-estradiol, and 0.5 mg of norethindrone acetate.

In some embodiments, the above fixed-dose composition is administered to the patient in one or more doses per 12 hours, 24 hours, 48 hours, 72 hours, week, month, or year, such as in from 1 to 10 doses per 12 hours (e.g., 1 dose every 12 hours, 2 doses every 12 hours, 3 doses every 12 hours, 4 doses every 12 hours, 5 doses every 12 hours, 6 doses every 12 hours, 7 doses every 12 hours, 8 doses every 12 hours, 9 doses every 12 hours, or 10 doses every 12 hours), from 1 to 10 doses per 24 hours (e.g., 1 dose every 24 hours, 2 doses every 24 hours, 3 doses every 24 hours, 4 doses every 24 hours, 5 doses every 24 hours, 6 doses every 24 hours, 7 doses every 24 hours, 8 doses every 24 hours, 9 doses every 24 hours, or 10 doses every 24 hours), from 1 to 10 doses per 48 hours (e.g., 1 dose every 48 hours, 2 doses every 48 hours, 3 doses every 48 hours, 4 doses every 48 hours, 5 doses every 48 hours, 6 doses every 48 hours, 7 doses every 48 hours, 8 doses every 48 hours, 9 doses every 48 hours, or 10 doses every 48 hours), from 1 to 10 doses per 72 hours (e.g., 1 dose every 72 hours, 2 doses every 72 hours, 3 doses every 72 hours, 4 doses every 72 hours, 5 doses every 72 hours, 6 doses every 72 hours, 7 doses every 72 hours, 8 doses every 72 hours, 9 doses every 72 hours, or 10 doses every 72 hours), from 1 to 10 doses per week (e.g., 1 dose every week, 2 doses every week, 3 doses every week, 4 doses every week, 5 doses every week, 6 doses every week, 7 doses every week, 8 doses every week, 9 doses every week, or 10 doses every week), or from 1 to 60 doses per month (e.g., from 30-60 doses per month, such as 1 time daily, 2 times daily, 3 times daily, 4 times daily, 5 times daily, 6 times daily, 7 times daily, 8 times daily, 9 times daily, 10 times daily, 7 times weekly, 8 times weekly, 9 times weekly, 10 times weekly, 11 times weekly, 12 times weekly, 13 times weekly, 14 times weekly, or more), among others. In some embodiments, the above fixed-dose composition is administered to the patient once daily.

In some embodiments, the add-back therapy includes from about 0.25 mg to about 0.75 mg of β17-estradiol, e.g., administered orally, and from about 0.05 mg to about 0.2 mg of norethindrone or a compound that is metabolized in vivo to produce norethindrone, such as an ester of norethindrone that is de-esterified in vivo to produce norethindrone, for instance, norethindrone acetate, e.g., administered orally. In some embodiments, the add-back therapy includes 0.5 mg of β17-estradiol, e.g., administered orally, and 0.1 mg of norethindrone or a compound that is metabolized in vivo to produce norethindrone, such as an ester of norethindrone that is de-esterified in vivo to produce norethindrone, for instance, norethindrone acetate, e.g., administered orally. In some embodiments, the add-back therapy includes 0.5 mg of β17-estradiol, e.g., administered orally, and, in the same pharmaceutical composition, 0.1 mg of norethindrone or a compound that is metabolized in vivo to produce norethindrone, such as an ester of norethindrone that is de-esterified in vivo to produce norethindrone, for instance, norethindrone acetate, e.g., administered orally. In some embodiments, the add-back therapy includes 0.5 mg of β17-estradiol, e.g., administered orally, and, in a separate pharmaceutical composition, 0.1 mg of norethindrone or a compound that is metabolized in vivo to produce norethindrone, such as an ester of norethindrone that is de-esterified in vivo to produce norethindrone, for instance, norethindrone acetate, e.g., administered orally.

In some embodiments, the GnRH antagonist is administered to the patient in a fixed-dose composition that contains the GnRH antagonist (e.g., in an amount of about 100 mg or about 200 mg of the compound of any one of formulas (I)-(VI)), from about 0.25 mg to about 0.75 mg of β17-estradiol, and from about 0.05 mg to about 0.2 mg of norethindrone or a compound that is metabolized in vivo to produce norethindrone, such as an ester of norethindrone that is de-esterified in vivo to produce norethindrone, for instance, norethindrone acetate. In some embodiments, the GnRH antagonist is administered to the patient in a fixed-dose composition that contains the GnRH antagonist (e.g., in an amount of about 100 mg or about 200 mg of the compound of any one of formulas (I)-(VI)), about 0.5 mg of β17-estradiol (e.g., 0.5 mg of β17-estradiol), and about 0.1 mg of norethindrone or a compound that is metabolized in vivo to produce norethindrone, such as an ester of norethindrone that is de-esterified in vivo to produce norethindrone, for instance, norethindrone acetate (e.g., 0.1 mg of norethindrone or a compound that is metabolized in vivo to produce norethindrone, such as an ester of norethindrone that is de-esterified in vivo to produce norethindrone, for instance, norethindrone acetate). In some embodiments, the GnRH antagonist is administered to the patient in a fixed-dose composition that contains the GnRH antagonist (e.g., in an amount of about 100 mg or about 200 mg of the compound of any one of formulas (I)-(VI)), 0.5 mg of β17-estradiol, and 0.1 mg of norethindrone acetate.

In some embodiments of any of the foregoing aspects (A1-A42) or embodiments of the disclosure, the patient is a pre-menopausal female of from about 18 to about 48 years of age, such as a patient of 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 years of age.

In some embodiments, the patient exhibits a serum concentration of FSH of about 20 IU/L or less prior to administration of the GnRH antagonist to the patient, such as a serum concentration of FSH of from about 5 IU/L to about 20 IU/L (e.g., a serum concentration of FSH of about 5 IU/L, 6 IU/L, 7 IU/L, 8 IU/L, 9 IU/L, 10 IU/L, 11 IU/L, 12 IU/L, 13 IU/L, 14 IU/L, 15 IU/L, 16 IU/L, 17 IU/L, 18 IU/L, 19 IU/L, or 20 μl/L.

In some embodiments, the patient exhibits a rectal (type II) and/or vaginal (type III) endometriosis node of at least 2 cm prior to administration of the GnRH antagonist to the patient. The length of the type II and/or type III endometriosis node may be assessed, for example, by way of magnetic resonance imaging (MRI).

In some embodiments, the patient exhibits a junctional-zone width of about 12 mm or more prior to administration of the GnRH antagonist to the patient, such as a junctional zone width of from about 12 mm to about 20 mm, or more (e.g., a junctional zone width of from about 12 mm to about 20 mm, from about 12 mm to about 19 mm, from about 12 mm to about 18 mm, from about 12 mm to about 17 mm, from about 12 mm to about 16 mm, from about 12 mm to about 15 mm, from about 12 mm to about 14 mm, or more) prior to administration of the GnRH antagonist to the patient. The junctional zone width may be assessed, for example, by way of MRI.

In some embodiments, the patient exhibits a reduction in serum concentration of FSH, LH, and/or E2 following administration of the GnRH antagonist to the patient. The reduction in serum concentration of LH, FSH, and/or E2 may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period).

In some embodiments, the patient exhibits a reduction in uterine bleeding following administration of the GnRH antagonist to the patient. The reduction in dyspareunia may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period). The reduction in uterine bleeding may be assessed by way of an alkaline hematin method, for example, as described herein.

In some embodiments, the patient exhibits amenorrhea following administration of the GnRH antagonist to the patient. The amenorrhea may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period).

In some embodiments, the patient exhibits a reduction in the volume of one or more rectovaginal endometriosis nodes following administration of the GnRH antagonist to the patient. The reduction in the volume of the one or more rectovaginal endometriosis nodes may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period). The reduction in the volume of the one or more rectovaginal endometriosis nodes may be assessed, for example, by way of MRI and/or TVUS.

In some embodiments, the patient exhibits a reduction in bowel involvement of one or more type III endometriosis nodes following administration of the GnRH antagonist to the patient. The reduction in bowel involvement of one or more type III endometriosis nodes may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period). The reduction in bowel involvement of one or more type III endometriosis nodes may be assessed, for example, by way of MRI.

In some embodiments, the patient exhibits a reduction in pelvic pain following administration of the GnRH antagonist to the patient. The reduction in pelvic pain may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period). The reduction in pelvic pain may be assessed by way of a modified Biberoglu & Behrman (mB&B) score, Numerical Rating Scale (NRS) score, or Verbal Rating Scale (VRS) score.

In some embodiments, the patient exhibits a reduction in dysmenorrhea following administration of the GnRH antagonist to the patient. The reduction in dysmenorrhea may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period). The reduction in dysmenorrhea may be assessed by way of an mB&B score, NRS score, or VRS score.

In some embodiments, the patient exhibits a reduction in dyspareunia following administration of the GnRH antagonist to the patient. The reduction in dyspareunia may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period). The reduction in dyspareunia may be assessed by way of an mB&B score, NRS score, or VRS score.

In some embodiments, the patient exhibits a reduction in dyschezia following administration of the GnRH antagonist to the patient. The reduction in dyschezia may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period). The reduction in dyschezia may be assessed by way of an mB&B score, NRS score, or VRS score.

In some embodiments, the patient exhibits a reduction in uterine volume following administration of the GnRH antagonist to the patient. The reduction in uterine volume may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period). The reduction in uterine volume may be assessed, for example, by way of MRI or transvaginal ultrasound (TVUS).

In some embodiments, the patient exhibits a reduction in the thickness of the anterior and/or posterior region of the uterine myometrium following administration of the GnRH antagonist to the patient. The reduction in the thickness of the anterior and/or posterior region of the uterine myometrium may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period).

In some embodiments, the patient exhibits a reduction in uterine tenderness following administration of the GnRH antagonist to the patient. The reduction in uterine tenderness may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period).

In some embodiments, the patient exhibits a reduction in the diameter of a junctional zone of adenomyosis following administration of the GnRH antagonist to the patient. The reduction in the diameter of a junctional zone of adenomyosis may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period).

In some embodiments, the patient exhibits an improvement in Endometriosis Health Profile questionnaire (EHP-30) score following administration of the GnRH antagonist to the patient. The improvement in the EHP-30 score may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period).

In some embodiments, the patient exhibits a positive Patient Global Impression of Change (PGIC) score following administration of the GnRH antagonist to the patient. The positive PGIC score may be effectuated within from about one day to about 36 weeks of commencement of the treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the treatment period (e.g., within from about 12 weeks to about 24 weeks of commencement of the treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the treatment period).

In some embodiments of any of the foregoing aspects (A1-A42) or embodiments of the disclosure, the method further includes monitoring the patient's bone mineral density at the end of the treatment period. For example, at the end of the treatment period, the patient's bone mineral density may be determined, e.g., using bone mineral density assessment techniques described herein, and may then be compared to a measurement of the patient's bone mineral density obtained prior to, or during, the treatment period.

In some embodiments of any of the foregoing aspects (A1-A42) or embodiments of the disclosure, the patient does not exhibit a reduction (e.g., a significant reduction) in bone mineral density at the end of the treatment period relative to a measurement of the patient's bone mineral density obtained prior to, or during, the treatment period. For example, in some embodiments, the patient does not exhibit a reduction in bone mineral density of greater than 5% at the end of the treatment period relative to a measurement of the patient's bone mineral density obtained prior to, or during, the treatment period. In some embodiments, the patient does not exhibit a reduction in bone mineral density of greater than 4% at the end of the treatment period relative to a measurement of the patient's bone mineral density obtained prior to, or during, the treatment period. In some embodiments, the patient does not exhibit a reduction in bone mineral density of greater than 3% at the end of the treatment period relative to a measurement of the patient's bone mineral density obtained prior to, or during, the treatment period. In some embodiments, the patient does not exhibit a reduction in bone mineral density of greater than 2% at the end of the treatment period relative to a measurement of the patient's bone mineral density obtained prior to, or during, the treatment period. In some embodiments, the patient does not exhibit a reduction in bone mineral density of greater than 1% at the end of the treatment period relative to a measurement of the patient's bone mineral density obtained prior to, or during, the treatment period.

In some embodiments of any of the foregoing aspects (A1-A42) or embodiments of the disclosure, the method includes determining that the patient does not exhibit a reduction (e.g., a significant reduction) in bone mineral density at the end of the treatment period relative to a measurement of the patient's bone mineral density obtained prior to, or during, the treatment period. For example, in some embodiments, the method includes determining that the patient does not exhibit a reduction in bone mineral density of greater than 5% at the end of the treatment period relative to a measurement of the patient's bone mineral density obtained prior to, or during, the treatment period. In some embodiments, the method includes determining that the patient does not exhibit a reduction in bone mineral density of greater than 4% at the end of the treatment period relative to a measurement of the patient's bone mineral density obtained prior to, or during, the treatment period. In some embodiments, the method includes determining that the patient does not exhibit a reduction in bone mineral density of greater than 3% at the end of the treatment period relative to a measurement of the patient's bone mineral density obtained prior to, or during, the treatment period. In some embodiments, the method includes determining that the patient does not exhibit a reduction in bone mineral density of greater than 2% at the end of the treatment period relative to a measurement of the patient's bone mineral density obtained prior to, or during, the treatment period. In some embodiments, the method includes determining that the patient does not exhibit a reduction in bone mineral density of greater than 1% at the end of the treatment period relative to a measurement of the patient's bone mineral density obtained prior to, or during, the treatment period.

Techniques for assessing bone mineral density that may be used in conjunction with the methods of the present disclosure include, for example, dual energy X-ray absorptiometry, such as in the spine and/or femur of the patient. In some embodiments, bone mineral density is assessed by comparing the concentration of bone specific alkaline phosphatase (BAP) in a sample isolated from the patient following the administration to the concentration of BAP in a sample isolated from the patient prior to administration of the GnRH antagonist. In some embodiments, the bone mineral density is assessed by comparing the concentration of deoxypyridinoline (DPD) in a sample isolated from the patient following the administration to the concentration of DPD in a sample isolated from the patient prior to administration of the GnRH antagonist. In some embodiments, the bone mineral density is assessed by comparing the concentration of type I collagen C-terminal telopeptide (CTX) in a sample isolated from the patient following the administration to the concentration of CTX in a sample isolated from the patient prior to administration of the GnRH antagonist. In some embodiments, the bone mineral density is assessed by comparing the concentration of procollagen 1 N-terminal peptide (P1NP) in a sample isolated from the patient following the administration to the concentration of P1NP in a sample isolated from the patient prior to administration of the GnRH antagonist.

In some embodiments of any of the foregoing aspects (A1-A42) or embodiments of the disclosure, the method includes monitoring the patient's total cholesterol level during the treatment period. The method may further include determining that the patient does not exhibit an increase (e.g., a significant increase) in total cholesterol level relative to a measurement of the patient's total cholesterol level obtained prior to the treatment period, and continuing to administer (e.g., periodically) the GnRH antagonist to the patient during the treatment period (e.g., for the remainder of the treatment period). For example, the method may include monitoring the patient's total cholesterol level after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in total cholesterol level (e.g., of greater than 20% (e.g., determining that the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1%)) relative to a measurement of the patient's total cholesterol level obtained prior to the treatment period, and continuing to administer (e.g., periodically) the GnRH antagonist to the patient during the treatment period (e.g., for the remainder of the treatment period). In some embodiments, the method includes monitoring the patient's total cholesterol level after 6 weeks of treatment with the GnRH antagonist, determining that the patient does not exhibit an increase in total cholesterol level of greater than 20% (e.g., determining that the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's total cholesterol level obtained prior to the treatment period, and continuing to administer (e.g., periodically) the GnRH antagonist to the patient during the treatment period (e.g., for the remainder of the treatment period). In some embodiments, the method includes monitoring the patient's total cholesterol level after 12 weeks of treatment with the GnRH antagonist, determining that the patient does not exhibit an increase in total cholesterol level of greater than 20% (e.g., determining that the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's total cholesterol level obtained prior to the treatment period, and continuing to administer (e.g., periodically) the GnRH antagonist to the patient during the treatment period (e.g., for the remainder of the treatment period). In some embodiments, the method includes monitoring the patient's total cholesterol level after 18 weeks of treatment with the GnRH antagonist, determining that the patient does not exhibit an increase in total cholesterol level of greater than 20% (e.g., determining that the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's total cholesterol level obtained prior to the treatment period, and continuing to administer (e.g., periodically) the GnRH antagonist to the patient during the treatment period (e.g., for the remainder of the treatment period). In some embodiments, the method includes monitoring the patient's total cholesterol level after 24 weeks of treatment with the GnRH antagonist, determining that the patient does not exhibit an increase in total cholesterol level of greater than 20% (e.g., determining that the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's total cholesterol level obtained prior to the treatment period, and continuing to administer (e.g., periodically) the GnRH antagonist to the patient during the treatment period (e.g., for the remainder of the treatment period). In some embodiments, the method includes monitoring the patient's total cholesterol level after 30 weeks of treatment with the GnRH antagonist, determining that the patient does not exhibit an increase in total cholesterol level of greater than 20% (e.g., determining that the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's total cholesterol level obtained prior to the treatment period, and continuing to administer (e.g., periodically) the GnRH antagonist to the patient during the treatment period (e.g., for the remainder of the treatment period). In some embodiments, the method includes monitoring the patient's total cholesterol level after 36 weeks of treatment with the GnRH antagonist, determining that the patient does not exhibit an increase in total cholesterol level of greater than 20% (e.g., determining that the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's total cholesterol level obtained prior to the treatment period, and continuing to administer (e.g., periodically) the GnRH antagonist to the patient during the treatment period (e.g., for the remainder of the treatment period). In some embodiments, the method includes monitoring the patient's total cholesterol level after 38 weeks of treatment with the GnRH antagonist, determining that the patient does not exhibit an increase in total cholesterol level of greater than 20% (e.g., determining that the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's total cholesterol level obtained prior to the treatment period, and continuing to administer (e.g., periodically) the GnRH antagonist to the patient during the treatment period (e.g., for the remainder of the treatment period). In some embodiments, the method includes monitoring the patient's total cholesterol level after 42 weeks of treatment with the GnRH antagonist, determining that the patient does not exhibit an increase in total cholesterol level of greater than 20% (e.g., determining that the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's total cholesterol level obtained prior to the treatment period, and continuing to administer (e.g., periodically) the GnRH antagonist to the patient during the treatment period (e.g., for the remainder of the treatment period). In some embodiments, the method includes monitoring the patient's total cholesterol level after 48 weeks of treatment with the GnRH antagonist, determining that the patient does not exhibit an increase in total cholesterol level of greater than 20% (e.g., determining that the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's total cholesterol level obtained prior to the treatment period, and continuing to administer (e.g., periodically) the GnRH antagonist to the patient during the treatment period (e.g., for the remainder of the treatment period). In some embodiments, the method includes monitoring the patient's total cholesterol level after 52 weeks of treatment with the GnRH antagonist, determining that the patient does not exhibit an increase in total cholesterol level of greater than 20% (e.g., determining that the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's total cholesterol level obtained prior to the treatment period, and continuing to administer (e.g., periodically) the GnRH antagonist to the patient during the treatment period (e.g., for the remainder of the treatment period).

In some embodiments of any of the foregoing aspects (A1-A42) or embodiments of the disclosure, the patient does not exhibit an increase (e.g., a significant increase) in total cholesterol level at the end of the treatment period relative to a measurement of the patient's total cholesterol level obtained prior to, or during, the treatment period. In some embodiments, the patient does not exhibit an increase in total cholesterol level (e.g., of greater than 20%) after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist (e.g., the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1% after from about 6 weeks to about 52 weeks of treatment with the GnRH antagonist) relative to a measurement of the patient's total cholesterol level obtained prior to the treatment period. In some embodiments, the patient does not exhibit an increase in total cholesterol level of greater than 20% after 6 weeks of treatment with the GnRH antagonist (e.g., the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1% after 6 weeks of treatment with the GnRH antagonist) relative to a measurement of the patient's total cholesterol level obtained prior to the treatment period. In some embodiments, the patient does not exhibit an increase in total cholesterol level of greater than 20% after 12 weeks of treatment with the GnRH antagonist (e.g., the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1% after 12 weeks of treatment with the GnRH antagonist) relative to a measurement of the patient's total cholesterol level obtained prior to the treatment period. In some embodiments, the patient does not exhibit an increase in total cholesterol level of greater than 20% after 18 weeks of treatment with the GnRH antagonist (e.g., the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1% after 18 weeks of treatment with the GnRH antagonist) relative to a measurement of the patient's total cholesterol level obtained prior to the treatment period. In some embodiments, the patient does not exhibit an increase in total cholesterol level of greater than 20% after 24 weeks of treatment with the GnRH antagonist (e.g., the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1% after 24 weeks of treatment with the GnRH antagonist) relative to a measurement of the patient's total cholesterol level obtained prior to the treatment period. In some embodiments, the patient does not exhibit an increase in total cholesterol level of greater than 20% after 30 weeks of treatment with the GnRH antagonist (e.g., the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1% after 30 weeks of treatment with the GnRH antagonist) relative to a measurement of the patient's total cholesterol level obtained prior to the treatment period. In some embodiments, the patient does not exhibit an increase in total cholesterol level of greater than 20% after 36 weeks of treatment with the GnRH antagonist (e.g., the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1% after 36 weeks of treatment with the GnRH antagonist) relative to a measurement of the patient's total cholesterol level obtained prior to the treatment period. In some embodiments, the patient does not exhibit an increase in total cholesterol level of greater than 20% after 38 weeks of treatment with the GnRH antagonist (e.g., the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1% after 38 weeks of treatment with the GnRH antagonist) relative to a measurement of the patient's total cholesterol level obtained prior to the treatment period. In some embodiments, the patient does not exhibit an increase in total cholesterol level of greater than 20% after 42 weeks of treatment with the GnRH antagonist (e.g., the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1% after 42 weeks of treatment with the GnRH antagonist) relative to a measurement of the patient's total cholesterol level obtained prior to the treatment period. In some embodiments, the patient does not exhibit an increase in total cholesterol level of greater than 20% after 48 weeks of treatment with the GnRH antagonist (e.g., the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1% after 48 weeks of treatment with the GnRH antagonist) relative to a measurement of the patient's total cholesterol level obtained prior to the treatment period. In some embodiments, the patient does not exhibit an increase in total cholesterol level of greater than 20% after 52 weeks of treatment with the GnRH antagonist (e.g., the patient does not exhibit an increase in total cholesterol level of greater than 20%, 19%, 18%, 17%, 16%, 15%, 14%, 13%, 12%, 11%, 10%, 9%, 8%, 7%, 6%, 5%, 4%, 3%, 2%, or 1% after 52 weeks of treatment with the GnRH antagonist) relative to a measurement of the patient's total cholesterol level obtained prior to the treatment period.

In some embodiments of any of the foregoing aspects (A1-A42) or embodiments of the disclosure, the method includes monitoring the patient's low-density lipoprotein-cholesterol level during the treatment period. The method may further include determining that the patient does not exhibit an increase (e.g., a significant increase) in low-density lipoprotein-cholesterol level relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period). For example, the method may include monitoring the patient's low-density lipoprotein-cholesterol level after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in low-density lipoprotein-cholesterol level (e.g., of greater than 40% (e.g., determining that the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%)) relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 6 weeks of treatment with the GnRH antagonist, determining that the patient does not exhibit an increase in low-density lipoprotein-cholesterol level of greater than 40% (e.g., determining that the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 12 weeks of treatment with the GnRH antagonist, determining that the patient does not exhibit an increase in low-density lipoprotein-cholesterol level of greater than 40% (e.g., determining that the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 18 weeks of treatment with the GnRH antagonist, determining that the patient does not exhibit an increase in low-density lipoprotein-cholesterol level of greater than 40% (e.g., determining that the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 24 weeks of treatment with the GnRH antagonist, determining that the patient does not exhibit an increase in low-density lipoprotein-cholesterol level of greater than 40% (e.g., determining that the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 30 weeks of treatment with the GnRH antagonist, determining that the patient does not exhibit an increase in low-density lipoprotein-cholesterol level of greater than 40% (e.g., determining that the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 36 weeks of treatment with the GnRH antagonist, determining that the patient does not exhibit an increase in low-density lipoprotein-cholesterol level of greater than 40% (e.g., determining that the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 38 weeks of treatment with the GnRH antagonist, determining that the patient does not exhibit an increase in low-density lipoprotein-cholesterol level of greater than 40% (e.g., determining that the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 42 weeks of treatment with the GnRH antagonist, determining that the patient does not exhibit an increase in low-density lipoprotein-cholesterol level of greater than 40% (e.g., determining that the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 48 weeks of treatment with the GnRH antagonist, determining that the patient does not exhibit an increase in low-density lipoprotein-cholesterol level of greater than 40% (e.g., determining that the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 52 weeks of treatment with the GnRH antagonist, determining that the patient does not exhibit an increase in low-density lipoprotein-cholesterol level of greater than 40% (e.g., determining that the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period).

In some embodiments of any of the foregoing aspects (A1-A42) or embodiments of the disclosure, the patient does not exhibit an increase (e.g., a significant increase) in low-density lipoprotein-cholesterol level at the end of the treatment period relative to a measurement of the patient's low-density lipoprotein-cholesterol level obtained prior to, or during, the treatment period. In some embodiments, the patient does not exhibit an increase in low-density lipoprotein-cholesterol level (e.g., of greater than 40%) after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist (e.g., the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after from about 6 weeks to about 52 weeks of treatment with the GnRH antagonist) relative to a measurement of the patient's low-density lipoprotein-cholesterol level obtained prior to the treatment period. In some embodiments, the patient does not exhibit an increase in low-density lipoprotein-cholesterol level of greater than 40% after 6 weeks of treatment with the GnRH antagonist (e.g., the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 6 weeks of treatment with the GnRH antagonist) relative to a measurement of the patient's low-density lipoprotein-cholesterol level obtained prior to the treatment period. In some embodiments, the patient does not exhibit an increase in low-density lipoprotein-cholesterol level of greater than 40% after 12 weeks of treatment with the GnRH antagonist (e.g., the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 12 weeks of treatment with the GnRH antagonist) relative to a measurement of the patient's low-density lipoprotein-cholesterol level obtained prior to the treatment period. In some embodiments, the patient does not exhibit an increase in low-density lipoprotein-cholesterol level of greater than 40% after 18 weeks of treatment with the GnRH antagonist (e.g., the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 18 weeks of treatment with the GnRH antagonist) relative to a measurement of the patient's low-density lipoprotein-cholesterol level obtained prior to the treatment period. In some embodiments, the patient does not exhibit an increase in low-density lipoprotein-cholesterol level of greater than 40% after 24 weeks of treatment with the GnRH antagonist (e.g., the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 24 weeks of treatment with the GnRH antagonist) relative to a measurement of the patient's low-density lipoprotein-cholesterol level obtained prior to the treatment period. In some embodiments, the patient does not exhibit an increase in low-density lipoprotein-cholesterol level of greater than 40% after 30 weeks of treatment with the GnRH antagonist (e.g., the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 30 weeks of treatment with the GnRH antagonist) relative to a measurement of the patient's low-density lipoprotein-cholesterol level obtained prior to the treatment period. In some embodiments, the patient does not exhibit an increase in low-density lipoprotein-cholesterol level of greater than 40% after 36 weeks of treatment with the GnRH antagonist (e.g., the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 36 weeks of treatment with the GnRH antagonist) relative to a measurement of the patient's low-density lipoprotein-cholesterol level obtained prior to the treatment period. In some embodiments, the patient does not exhibit an increase in low-density lipoprotein-cholesterol level of greater than 40% after 38 weeks of treatment with the GnRH antagonist (e.g., the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 38 weeks of treatment with the GnRH antagonist) relative to a measurement of the patient's low-density lipoprotein-cholesterol level obtained prior to the treatment period. In some embodiments, the patient does not exhibit an increase in low-density lipoprotein-cholesterol level of greater than 40% after 42 weeks of treatment with the GnRH antagonist (e.g., the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 42 weeks of treatment with the GnRH antagonist) relative to a measurement of the patient's low-density lipoprotein-cholesterol level obtained prior to the treatment period. In some embodiments, the patient does not exhibit an increase in low-density lipoprotein-cholesterol level of greater than 40% after 48 weeks of treatment with the GnRH antagonist (e.g., the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 48 weeks of treatment with the GnRH antagonist) relative to a measurement of the patient's low-density lipoprotein-cholesterol level obtained prior to the treatment period. In some embodiments, the patient does not exhibit an increase in low-density lipoprotein-cholesterol level of greater than 40% after 52 weeks of treatment with the GnRH antagonist (e.g., the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 52 weeks of treatment with the GnRH antagonist) relative to a measurement of the patient's low-density lipoprotein-cholesterol level obtained prior to the treatment period.

In some embodiments of any of the foregoing aspects (A1-A42) or embodiments of the disclosure, the method includes monitoring the patient's low-density lipoprotein-cholesterol level after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in low-density lipoprotein-cholesterol level to a level greater than 160 mg/dl (e.g., determining that the patient does not exhibit an increase in low-density lipoprotein-cholesterol level to a level greater than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl), and continuing to administer (e.g., periodically) the GnRH antagonist to the patient during the treatment period (e.g., for the remainder of the treatment period). In some embodiments, the method includes monitoring the patient's low-density lipoprotein-cholesterol level after 6 weeks of treatment with the GnRH antagonist, determining that the patient does not exhibit an increase in low-density lipoprotein-cholesterol level to a level greater than 160 mg/dl (e.g., determining that the patient does not exhibit an increase in low-density lipoprotein-cholesterol level to a level greater than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl), and continuing to administer (e.g., periodically) the GnRH antagonist to the patient during the treatment period (e.g., for the remainder of the treatment period). In some embodiments, the method includes monitoring the patient's low-density lipoprotein-cholesterol level after 12 weeks of treatment with the GnRH antagonist, determining that the patient does not exhibit an increase in low-density lipoprotein-cholesterol level to a level greater than 160 mg/dl (e.g., determining that the patient does not exhibit an increase in low-density lipoprotein-cholesterol level to a level greater than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl), and continuing to administer (e.g., periodically) the GnRH antagonist to the patient during the treatment period (e.g., for the remainder of the treatment period). In some embodiments, the method includes monitoring the patient's low-density lipoprotein-cholesterol level after 18 weeks of treatment with the GnRH antagonist, determining that the patient does not exhibit an increase in low-density lipoprotein-cholesterol level to a level greater than 160 mg/dl (e.g., determining that the patient does not exhibit an increase in low-density lipoprotein-cholesterol level to a level greater than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl), and continuing to administer (e.g., periodically) the GnRH antagonist to the patient during the treatment period (e.g., for the remainder of the treatment period). In some embodiments, the method includes monitoring the patient's low-density lipoprotein-cholesterol level after 24 weeks of treatment with the GnRH antagonist, determining that the patient does not exhibit an increase in low-density lipoprotein-cholesterol level to a level greater than 160 mg/dl (e.g., determining that the patient does not exhibit an increase in low-density lipoprotein-cholesterol level to a level greater than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl), and continuing to administer (e.g., periodically) the GnRH antagonist to the patient during the treatment period (e.g., for the remainder of the treatment period). In some embodiments, the method includes monitoring the patient's low-density lipoprotein-cholesterol level after 30 weeks of treatment with the GnRH antagonist, determining that the patient does not exhibit an increase in low-density lipoprotein-cholesterol level to a level greater than 160 mg/dl (e.g., determining that the patient does not exhibit an increase in low-density lipoprotein-cholesterol level to a level greater than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl), and continuing to administer (e.g., periodically) the GnRH antagonist to the patient during the treatment period (e.g., for the remainder of the treatment period). In some embodiments, the method includes monitoring the patient's low-density lipoprotein-cholesterol level after 36 weeks of treatment with the GnRH antagonist, determining that the patient does not exhibit an increase in low-density lipoprotein-cholesterol level to a level greater than 160 mg/dl (e.g., determining that the patient does not exhibit an increase in low-density lipoprotein-cholesterol level to a level greater than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl), and continuing to administer (e.g., periodically) the GnRH antagonist to the patient during the treatment period (e.g., for the remainder of the treatment period). In some embodiments, the method includes monitoring the patient's low-density lipoprotein-cholesterol level after 38 weeks of treatment with the GnRH antagonist, determining that the patient does not exhibit an increase in low-density lipoprotein-cholesterol level to a level greater than 160 mg/dl (e.g., determining that the patient does not exhibit an increase in low-density lipoprotein-cholesterol level to a level greater than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl), and continuing to administer (e.g., periodically) the GnRH antagonist to the patient during the treatment period (e.g., for the remainder of the treatment period). In some embodiments, the method includes monitoring the patient's low-density lipoprotein-cholesterol level after 42 weeks of treatment with the GnRH antagonist, determining that the patient does not exhibit an increase in low-density lipoprotein-cholesterol level to a level greater than 160 mg/dl (e.g., determining that the patient does not exhibit an increase in low-density lipoprotein-cholesterol level to a level greater than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl), and continuing to administer (e.g., periodically) the GnRH antagonist to the patient during the treatment period (e.g., for the remainder of the treatment period). In some embodiments, the method includes monitoring the patient's low-density lipoprotein-cholesterol level after 48 weeks of treatment with the GnRH antagonist, determining that the patient does not exhibit an increase in low-density lipoprotein-cholesterol level to a level greater than 160 mg/dl (e.g., determining that the patient does not exhibit an increase in low-density lipoprotein-cholesterol level to a level greater than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl), and continuing to administer (e.g., periodically) the GnRH antagonist to the patient during the treatment period (e.g., for the remainder of the treatment period). In some embodiments, the method includes monitoring the patient's low-density lipoprotein-cholesterol level after 52 weeks of treatment with the GnRH antagonist, determining that the patient does not exhibit an increase in low-density lipoprotein-cholesterol level to a level greater than 160 mg/dl (e.g., determining that the patient does not exhibit an increase in low-density lipoprotein-cholesterol level to a level greater than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl), and continuing to administer (e.g., periodically) the GnRH antagonist to the patient during the treatment period (e.g., for the remainder of the treatment period).

In some embodiments of any of the foregoing aspects (A1-A42) or embodiments of the disclosure, the patient does not exhibit an increase in low-density lipoprotein-cholesterol level to a level greater than 160 mg/dl at the end of the treatment period. In some embodiments, the patient does not exhibit an increase in low-density lipoprotein-cholesterol level to a level greater than 160 mg/dl after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist. In some embodiments, the patient does not exhibit an increase in low-density lipoprotein-cholesterol level to a level greater than 160 mg/dl after 6 weeks of treatment with the GnRH antagonist. In some embodiments, the patient does not exhibit an increase in low-density lipoprotein-cholesterol level to a level greater than 160 mg/dl after 12 weeks of treatment with the GnRH antagonist. In some embodiments, the patient does not exhibit an increase in low-density lipoprotein-cholesterol level to a level greater than 160 mg/dl after 18 weeks of treatment with the GnRH antagonist. In some embodiments, the patient does not exhibit an increase in low-density lipoprotein-cholesterol level to a level greater than 160 mg/dl after 24 weeks of treatment with the GnRH antagonist. In some embodiments, the patient does not exhibit an increase in low-density lipoprotein-cholesterol level to a level greater than 160 mg/dl after 30 weeks of treatment with the GnRH antagonist. In some embodiments, the patient does not exhibit an increase in low-density lipoprotein-cholesterol level to a level greater than 160 mg/dl after 36 weeks of treatment with the GnRH antagonist. In some embodiments, the patient does not exhibit an increase in low-density lipoprotein-cholesterol level to a level greater than 160 mg/dl after 38 weeks of treatment with the GnRH antagonist. In some embodiments, the patient does not exhibit an increase in low-density lipoprotein-cholesterol level to a level greater than 160 mg/dl after 42 weeks of treatment with the GnRH antagonist. In some embodiments, the patient does not exhibit an increase in low-density lipoprotein-cholesterol level to a level greater than 160 mg/dl after 48 weeks of treatment with the GnRH antagonist. In some embodiments, the patient does not exhibit an increase in low-density lipoprotein-cholesterol level to a level greater than 160 mg/dl after 52 weeks of treatment with the GnRH antagonist.

In some embodiments of any of the foregoing aspects (A1-A42) or embodiments of the disclosure, the method includes monitoring the patient's low-density lipoprotein-cholesterol level after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in low-density lipoprotein-cholesterol level from a level less than 160 mg/dl (e.g., from a level less than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl) to a level greater than 190 mg/dl, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 6 weeks of treatment with the GnRH antagonist, determining that the patient does not exhibit an increase in low-density lipoprotein-cholesterol level from a level less than 160 mg/dl (e.g., from a level less than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl) to a level greater than 190 mg/dl, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 12 weeks of treatment with the GnRH antagonist, determining that the patient does not exhibit an increase in low-density lipoprotein-cholesterol level from a level less than 160 mg/dl (e.g., from a level less than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl) to a level greater than 190 mg/dl, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 18 weeks of treatment with the GnRH antagonist, determining that the patient does not exhibit an increase in low-density lipoprotein-cholesterol level from a level less than 160 mg/dl (e.g., from a level less than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl) to a level greater than 190 mg/dl, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 24 weeks of treatment with the GnRH antagonist, determining that the patient does not exhibit an increase in low-density lipoprotein-cholesterol level from a level less than 160 mg/dl (e.g., from a level less than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl) to a level greater than 190 mg/dl, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 30 weeks of treatment with the GnRH antagonist, determining that the patient does not exhibit an increase in low-density lipoprotein-cholesterol level from a level less than 160 mg/dl (e.g., from a level less than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl) to a level greater than 190 mg/dl, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 36 weeks of treatment with the GnRH antagonist, determining that the patient does not exhibit an increase in low-density lipoprotein-cholesterol level from a level less than 160 mg/dl (e.g., from a level less than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl) to a level greater than 190 mg/dl, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 38 weeks of treatment with the GnRH antagonist, determining that the patient does not exhibit an increase in low-density lipoprotein-cholesterol level from a level less than 160 mg/dl (e.g., from a level less than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl) to a level greater than 190 mg/dl, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 42 weeks of treatment with the GnRH antagonist, determining that the patient does not exhibit an increase in low-density lipoprotein-cholesterol level from a level less than 160 mg/dl (e.g., from a level less than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl) to a level greater than 190 mg/dl, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 48 weeks of treatment with the GnRH antagonist, determining that the patient does not exhibit an increase in low-density lipoprotein-cholesterol level from a level less than 160 mg/dl (e.g., from a level less than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl) to a level greater than 190 mg/dl, and continuing to administer (e.g., periodically) the GnRH antagonist to the patient during the treatment period (e.g., for the remainder of the treatment period). In some embodiments, the method includes monitoring the patient's low-density lipoprotein-cholesterol level after 52 weeks of treatment with the GnRH antagonist, determining that the patient does not exhibit an increase in low-density lipoprotein-cholesterol level from a level less than 160 mg/dl (e.g., from a level less than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl) to a level greater than 190 mg/dl, and continuing to administer (e.g., periodically) the GnRH antagonist to the patient during the treatment period (e.g., for the remainder of the treatment period).

In some embodiments of any of the foregoing aspects (A1-A42) or embodiments of the disclosure, the patient does not exhibit an increase in low-density lipoprotein-cholesterol level from a level less than 160 mg/dl to a level greater than 190 mg/dl at the end of the treatment period. In some embodiments, the patient does not exhibit an increase in low-density lipoprotein-cholesterol level from a level less than 160 mg/dl to a level greater than 190 mg/dl after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist. In some embodiments, the patient does not exhibit an increase in low-density lipoprotein-cholesterol level from a level less than 160 mg/dl to a level greater than 190 mg/dl after 6 weeks of treatment with the GnRH antagonist. In some embodiments, the patient does not exhibit an increase in low-density lipoprotein-cholesterol level from a level less than 160 mg/dl to a level greater than 190 mg/dl after 12 weeks of treatment with the GnRH antagonist. In some embodiments, the patient does not exhibit an increase in low-density lipoprotein-cholesterol level from a level less than 160 mg/dl to a level greater than 190 mg/dl after 18 weeks of treatment with the GnRH antagonist. In some embodiments, the patient does not exhibit an increase in low-density lipoprotein-cholesterol level from a level less than 160 mg/dl to a level greater than 190 mg/dl after 24 weeks of treatment with the GnRH antagonist. In some embodiments, the patient does not exhibit an increase in low-density lipoprotein-cholesterol level from a level less than 160 mg/dl to a level greater than 190 mg/dl after 30 weeks of treatment with the GnRH antagonist. In some embodiments, the patient does not exhibit an increase in low-density lipoprotein-cholesterol level from a level less than 160 mg/dl to a level greater than 190 mg/dl after 36 weeks of treatment with the GnRH antagonist. In some embodiments, the patient does not exhibit an increase in low-density lipoprotein-cholesterol level from a level less than 160 mg/dl to a level greater than 190 mg/dl after 38 weeks of treatment with the GnRH antagonist. In some embodiments, the patient does not exhibit an increase in low-density lipoprotein-cholesterol level from a level less than 160 mg/dl to a level greater than 190 mg/dl after 42 weeks of treatment with the GnRH antagonist. In some embodiments, the patient does not exhibit an increase in low-density lipoprotein-cholesterol level from a level less than 160 mg/dl to a level greater than 190 mg/dl after 48 weeks of treatment with the GnRH antagonist. In some embodiments, the patient does not exhibit an increase in low-density lipoprotein-cholesterol level from a level less than 160 mg/dl to a level greater than 190 mg/dl after 52 weeks of treatment with the GnRH antagonist.

For example, in some embodiments of any of the foregoing aspects (A1-A42) or embodiments of the disclosure, the patient does not exhibit an increase in low-density lipoprotein-cholesterol level from a level less than 130 mg/dl to a level greater than 190 mg/dl at the end of the treatment period. In some embodiments, the patient does not exhibit an increase in low-density lipoprotein-cholesterol level from a level less than 130 mg/dl to a level greater than 190 mg/dl after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist. In some embodiments, the patient does not exhibit an increase in low-density lipoprotein-cholesterol level from a level less than 130 mg/dl to a level greater than 190 mg/dl after 6 weeks of treatment with the GnRH antagonist. In some embodiments, the patient does not exhibit an increase in low-density lipoprotein-cholesterol level from a level less than 130 mg/dl to a level greater than 190 mg/dl after 12 weeks of treatment with the GnRH antagonist. In some embodiments, the patient does not exhibit an increase in low-density lipoprotein-cholesterol level from a level less than 130 mg/dl to a level greater than 190 mg/dl after 18 weeks of treatment with the GnRH antagonist. In some embodiments, the patient does not exhibit an increase in low-density lipoprotein-cholesterol level from a level less than 130 mg/dl to a level greater than 190 mg/dl after 24 weeks of treatment with the GnRH antagonist. In some embodiments, the patient does not exhibit an increase in low-density lipoprotein-cholesterol level from a level less than 130 mg/dl to a level greater than 190 mg/dl after 30 weeks of treatment with the GnRH antagonist. In some embodiments, the patient does not exhibit an increase in low-density lipoprotein-cholesterol level from a level less than 130 mg/dl to a level greater than 190 mg/dl after 36 weeks of treatment with the GnRH antagonist. In some embodiments, the patient does not exhibit an increase in low-density lipoprotein-cholesterol level from a level less than 130 mg/dl to a level greater than 190 mg/dl after 38 weeks of treatment with the GnRH antagonist. In some embodiments, the patient does not exhibit an increase in low-density lipoprotein-cholesterol level from a level less than 130 mg/dl to a level greater than 190 mg/dl after 42 weeks of treatment with the GnRH antagonist. In some embodiments, the patient does not exhibit an increase in low-density lipoprotein-cholesterol level from a level less than 130 mg/dl to a level greater than 190 mg/dl after 48 weeks of treatment with the GnRH antagonist. In some embodiments, the patient does not exhibit an increase in low-density lipoprotein-cholesterol level from a level less than 130 mg/dl to a level greater than 190 mg/dl after 52 weeks of treatment with the GnRH antagonist.

In some embodiments of any of the foregoing aspects (A1-A42) or embodiments of the disclosure, the method includes monitoring the patient's ratio of low-density lipoprotein-cholesterol to high-density lipoprotein-cholesterol during the treatment period. The method may further include determining that the patient does not exhibit an increase (e.g., a significant increase) in their ratio of low-density lipoprotein-cholesterol to high-density lipoprotein-cholesterol relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period). In some embodiments, the method includes monitoring the patient's ratio of low-density lipoprotein-cholesterol to high-density lipoprotein-cholesterol after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in their ratio of low-density lipoprotein-cholesterol to high-density lipoprotein-cholesterol (e.g., of greater than 40% (e.g., determining that the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%)) relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period). In some embodiments, the method includes monitoring the patient's ratio of low-density lipoprotein-cholesterol to high-density lipoprotein-cholesterol after 6 weeks of treatment with the GnRH antagonist, determining that the patient does not exhibit an increase in their ratio of low-density lipoprotein-cholesterol to high-density lipoprotein-cholesterol of greater than 40% (e.g., determining that the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period). In some embodiments, the method includes monitoring the patient's ratio of low-density lipoprotein-cholesterol to high-density lipoprotein-cholesterol after 12 weeks of treatment with the GnRH antagonist, determining that the patient does not exhibit an increase in their ratio of low-density lipoprotein-cholesterol to high-density lipoprotein-cholesterol of greater than 40% (e.g., determining that the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period). In some embodiments, the method includes monitoring the patient's ratio of low-density lipoprotein-cholesterol to high-density lipoprotein-cholesterol after 18 weeks of treatment with the GnRH antagonist, determining that the patient does not exhibit an increase in their ratio of low-density lipoprotein-cholesterol to high-density lipoprotein-cholesterol of greater than 40% (e.g., determining that the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period). In some embodiments, the method includes monitoring the patient's ratio of low-density lipoprotein-cholesterol to high-density lipoprotein-cholesterol after 24 weeks of treatment with the GnRH antagonist, determining that the patient does not exhibit an increase in their ratio of low-density lipoprotein-cholesterol to high-density lipoprotein-cholesterol of greater than 40% (e.g., determining that the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period). In some embodiments, the method includes monitoring the patient's ratio of low-density lipoprotein-cholesterol to high-density lipoprotein-cholesterol after 30 weeks of treatment with the GnRH antagonist, determining that the patient does not exhibit an increase in their ratio of low-density lipoprotein-cholesterol to high-density lipoprotein-cholesterol of greater than 40% (e.g., determining that the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period). In some embodiments, the method includes monitoring the patient's ratio of low-density lipoprotein-cholesterol to high-density lipoprotein-cholesterol after 36 weeks of treatment with the GnRH antagonist, determining that the patient does not exhibit an increase in their ratio of low-density lipoprotein-cholesterol to high-density lipoprotein-cholesterol of greater than 40% (e.g., determining that the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period). In some embodiments, the method includes monitoring the patient's ratio of low-density lipoprotein-cholesterol to high-density lipoprotein-cholesterol after 38 weeks of treatment with the GnRH antagonist, determining that the patient does not exhibit an increase in their ratio of low-density lipoprotein-cholesterol to high-density lipoprotein-cholesterol of greater than 40% (e.g., determining that the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period). In some embodiments, the method includes monitoring the patient's ratio of low-density lipoprotein-cholesterol to high-density lipoprotein-cholesterol after 42 weeks of treatment with the GnRH antagonist, determining that the patient does not exhibit an increase in their ratio of low-density lipoprotein-cholesterol to high-density lipoprotein-cholesterol of greater than 40% (e.g., determining that the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period). In some embodiments, the method includes monitoring the patient's ratio of low-density lipoprotein-cholesterol to high-density lipoprotein-cholesterol after 48 weeks of treatment with the GnRH antagonist, determining that the patient does not exhibit an increase in their ratio of low-density lipoprotein-cholesterol to high-density lipoprotein-cholesterol of greater than 40% (e.g., determining that the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period). In some embodiments, the method includes monitoring the patient's ratio of low-density lipoprotein-cholesterol to high-density lipoprotein-cholesterol after 52 weeks of treatment with the GnRH antagonist, determining that the patient does not exhibit an increase in their ratio of low-density lipoprotein-cholesterol to high-density lipoprotein-cholesterol of greater than 40% (e.g., determining that the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period).

In some embodiments of any of the foregoing aspects (A1-A42) or embodiments of the disclosure, the patient does not exhibit an increase (e.g., a significant increase) in their ratio of low-density lipoprotein-cholesterol to high-density lipoprotein-cholesterol at the end of the treatment period relative to a measurement of their ratio of low-density lipoprotein-cholesterol to high-density lipoprotein-cholesterol obtained prior to, or during, the treatment period. In some embodiments, the patient does not exhibit an increase in their ratio of low-density lipoprotein-cholesterol to high-density lipoprotein-cholesterol (e.g., of greater than 40%) after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist (e.g., the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after from about 6 weeks to about 52 weeks of treatment with the GnRH antagonist) relative to a measurement of their ratio of low-density lipoprotein-cholesterol to high-density lipoprotein-cholesterol obtained prior to the treatment period. In some embodiments, the patient does not exhibit an increase in their ratio of low-density lipoprotein-cholesterol to high-density lipoprotein-cholesterol of greater than 40% after 6 weeks of treatment with the GnRH antagonist (e.g., the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 6 weeks of treatment with the GnRH antagonist) relative to a measurement of their ratio of low-density lipoprotein-cholesterol to high-density lipoprotein-cholesterol obtained prior to the treatment period. In some embodiments, the patient does not exhibit an increase in their ratio of low-density lipoprotein-cholesterol to high-density lipoprotein-cholesterol of greater than 40% after 12 weeks of treatment with the GnRH antagonist (e.g., the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 12 weeks of treatment with the GnRH antagonist) relative to a measurement of their ratio of low-density lipoprotein-cholesterol to high-density lipoprotein-cholesterol obtained prior to the treatment period. In some embodiments, the patient does not exhibit an increase in their ratio of low-density lipoprotein-cholesterol to high-density lipoprotein-cholesterol of greater than 40% after 18 weeks of treatment with the GnRH antagonist (e.g., the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 18 weeks of treatment with the GnRH antagonist) relative to a measurement of their ratio of low-density lipoprotein-cholesterol to high-density lipoprotein-cholesterol obtained prior to the treatment period. In some embodiments, the patient does not exhibit an increase in their ratio of low-density lipoprotein-cholesterol to high-density lipoprotein-cholesterol of greater than 40% after 24 weeks of treatment with the GnRH antagonist (e.g., the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 24 weeks of treatment with the GnRH antagonist) relative to a measurement of their ratio of low-density lipoprotein-cholesterol to high-density lipoprotein-cholesterol obtained prior to the treatment period. In some embodiments, the patient does not exhibit an increase in their ratio of low-density lipoprotein-cholesterol to high-density lipoprotein-cholesterol of greater than 40% after 30 weeks of treatment with the GnRH antagonist (e.g., the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 30 weeks of treatment with the GnRH antagonist) relative to a measurement of their ratio of low-density lipoprotein-cholesterol to high-density lipoprotein-cholesterol obtained prior to the treatment period. In some embodiments, the patient does not exhibit an increase in their ratio of low-density lipoprotein-cholesterol to high-density lipoprotein-cholesterol of greater than 40% after 36 weeks of treatment with the GnRH antagonist (e.g., the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 36 weeks of treatment with the GnRH antagonist) relative to a measurement of their ratio of low-density lipoprotein-cholesterol to high-density lipoprotein-cholesterol obtained prior to the treatment period. In some embodiments, the patient does not exhibit an increase in their ratio of low-density lipoprotein-cholesterol to high-density lipoprotein-cholesterol of greater than 40% after 38 weeks of treatment with the GnRH antagonist (e.g., the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 38 weeks of treatment with the GnRH antagonist) relative to a measurement of their ratio of low-density lipoprotein-cholesterol to high-density lipoprotein-cholesterol obtained prior to the treatment period. In some embodiments, the patient does not exhibit an increase in their ratio of low-density lipoprotein-cholesterol to high-density lipoprotein-cholesterol of greater than 40% after 42 weeks of treatment with the GnRH antagonist (e.g., the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 42 weeks of treatment with the GnRH antagonist) relative to a measurement of their ratio of low-density lipoprotein-cholesterol to high-density lipoprotein-cholesterol obtained prior to the treatment period. In some embodiments, the patient does not exhibit an increase in their ratio of low-density lipoprotein-cholesterol to high-density lipoprotein-cholesterol of greater than 40% after 48 weeks of treatment with the GnRH antagonist (e.g., the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 48 weeks of treatment with the GnRH antagonist) relative to a measurement of their ratio of low-density lipoprotein-cholesterol to high-density lipoprotein-cholesterol obtained prior to the treatment period. In some embodiments, the patient does not exhibit an increase in their ratio of low-density lipoprotein-cholesterol to high-density lipoprotein-cholesterol of greater than 40% after 52 weeks of treatment with the GnRH antagonist (e.g., the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 52 weeks of treatment with the GnRH antagonist) relative to a measurement of their ratio of low-density lipoprotein-cholesterol to high-density lipoprotein-cholesterol obtained prior to the treatment period.

In some embodiments of any of the foregoing aspects (A1-A42) or embodiments of the disclosure, the method includes monitoring the patient's serum triglyceride level during the treatment period. The method may further include determining that the patient does not exhibit an increase (e.g., a significant increase) in serum triglyceride level relative to a measurement of the patient's triglyceride level obtained prior to the treatment period, and continuing to administer (e.g., periodically) the GnRH antagonist to the patient during the treatment period (e.g., for the remainder of the treatment period). In some embodiments, the method includes monitoring the patient's serum triglyceride level after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in serum triglyceride level (e.g., of greater than 50% (e.g., determining that the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%)) relative to a measurement of the patient's triglyceride level obtained prior to the treatment period, and continuing to administer (e.g., periodically) the GnRH antagonist to the patient during the treatment period (e.g., for the remainder of the treatment period). In some embodiments, the method includes monitoring the patient's serum triglyceride level after 6 weeks of treatment with the GnRH antagonist, determining that the patient does not exhibit an increase in serum triglyceride level of greater than 50% (e.g., determining that the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's triglyceride level obtained prior to the treatment period, and continuing to administer (e.g., periodically) the GnRH antagonist to the patient during the treatment period (e.g., for the remainder of the treatment period). In some embodiments, the method includes monitoring the patient's serum triglyceride level after 12 weeks of treatment with the GnRH antagonist, determining that the patient does not exhibit an increase in serum triglyceride level of greater than 50% (e.g., determining that the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's triglyceride level obtained prior to the treatment period, and continuing to administer (e.g., periodically) the GnRH antagonist to the patient during the treatment period (e.g., for the remainder of the treatment period). In some embodiments, the method includes monitoring the patient's serum triglyceride level after 18 weeks of treatment with the GnRH antagonist, determining that the patient does not exhibit an increase in serum triglyceride level of greater than 50% (e.g., determining that the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's triglyceride level obtained prior to the treatment period, and continuing to administer (e.g., periodically) the GnRH antagonist to the patient during the treatment period (e.g., for the remainder of the treatment period). In some embodiments, the method includes monitoring the patient's serum triglyceride level after 24 weeks of treatment with the GnRH antagonist, determining that the patient does not exhibit an increase in serum triglyceride level of greater than 50% (e.g., determining that the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's triglyceride level obtained prior to the treatment period, and continuing to administer (e.g., periodically) the GnRH antagonist to the patient during the treatment period (e.g., for the remainder of the treatment period). In some embodiments, the method includes monitoring the patient's serum triglyceride level after 30 weeks of treatment with the GnRH antagonist, determining that the patient does not exhibit an increase in serum triglyceride level of greater than 50% (e.g., determining that the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's triglyceride level obtained prior to the treatment period, and continuing to administer (e.g., periodically) the GnRH antagonist to the patient during the treatment period (e.g., for the remainder of the treatment period). In some embodiments, the method includes monitoring the patient's serum triglyceride level after 36 weeks of treatment with the GnRH antagonist, determining that the patient does not exhibit an increase in serum triglyceride level of greater than 50% (e.g., determining that the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's triglyceride level obtained prior to the treatment period, and continuing to administer (e.g., periodically) the GnRH antagonist to the patient during the treatment period (e.g., for the remainder of the treatment period). In some embodiments, the method includes monitoring the patient's serum triglyceride level after 38 weeks of treatment with the GnRH antagonist, determining that the patient does not exhibit an increase in serum triglyceride level of greater than 50% (e.g., determining that the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's triglyceride level obtained prior to the treatment period, and continuing to administer (e.g., periodically) the GnRH antagonist to the patient during the treatment period (e.g., for the remainder of the treatment period). In some embodiments, the method includes monitoring the patient's serum triglyceride level after 42 weeks of treatment with the GnRH antagonist, determining that the patient does not exhibit an increase in serum triglyceride level of greater than 50% (e.g., determining that the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's triglyceride level obtained prior to the treatment period, and continuing to administer (e.g., periodically) the GnRH antagonist to the patient during the treatment period (e.g., for the remainder of the treatment period). In some embodiments, the method includes monitoring the patient's serum triglyceride level after 48 weeks of treatment with the GnRH antagonist, determining that the patient does not exhibit an increase in serum triglyceride level of greater than 50% (e.g., determining that the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's triglyceride level obtained prior to the treatment period, and continuing to administer (e.g., periodically) the GnRH antagonist to the patient during the treatment period (e.g., for the remainder of the treatment period). In some embodiments, the method includes monitoring the patient's serum triglyceride level after 52 weeks of treatment with the GnRH antagonist, determining that the patient does not exhibit an increase in serum triglyceride level of greater than 50% (e.g., determining that the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) relative to a measurement of the patient's triglyceride level obtained prior to the treatment period, and continuing to administer (e.g., periodically) the GnRH antagonist to the patient during the treatment period (e.g., for the remainder of the treatment period).

In some embodiments of any of the foregoing aspects (A1-A42) or embodiments of the disclosure, the patient does not exhibit an increase (e.g., a significant increase) in serum triglyceride level at the end of the treatment period relative to a measurement of the patient's triglyceride level obtained prior to, or during, the treatment period. In some embodiments, the patient does not exhibit an increase in serum triglyceride level (e.g., of greater than 50%) after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist (e.g., the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after from about 6 weeks to about 52 weeks of treatment with the GnRH antagonist) relative to a measurement of the patient's triglyceride level obtained prior to the treatment period. In some embodiments, the patient does not exhibit an increase in serum triglyceride level of greater than 50% after 6 weeks of treatment with the GnRH antagonist (e.g., the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 6 weeks of treatment with the GnRH antagonist) relative to a measurement of the patient's triglyceride level obtained prior to the treatment period. In some embodiments, the patient does not exhibit an increase in serum triglyceride level of greater than 50% after 12 weeks of treatment with the GnRH antagonist (e.g., the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 12 weeks of treatment with the GnRH antagonist) relative to a measurement of the patient's triglyceride level obtained prior to the treatment period. In some embodiments, the patient does not exhibit an increase in serum triglyceride level of greater than 50% after 18 weeks of treatment with the GnRH antagonist (e.g., the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 18 weeks of treatment with the GnRH antagonist) relative to a measurement of the patient's triglyceride level obtained prior to the treatment period. In some embodiments, the patient does not exhibit an increase in serum triglyceride level of greater than 50% after 24 weeks of treatment with the GnRH antagonist (e.g., the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 24 weeks of treatment with the GnRH antagonist) relative to a measurement of the patient's triglyceride level obtained prior to the treatment period. In some embodiments, the patient does not exhibit an increase in serum triglyceride level of greater than 50% after 30 weeks of treatment with the GnRH antagonist (e.g., the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 30 weeks of treatment with the GnRH antagonist) relative to a measurement of the patient's triglyceride level obtained prior to the treatment period. In some embodiments, the patient does not exhibit an increase in serum triglyceride level of greater than 50% after 36 weeks of treatment with the GnRH antagonist (e.g., the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 36 weeks of treatment with the GnRH antagonist) relative to a measurement of the patient's triglyceride level obtained prior to the treatment period. In some embodiments, the patient does not exhibit an increase in serum triglyceride level of greater than 50% after 38 weeks of treatment with the GnRH antagonist (e.g., the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 38 weeks of treatment with the GnRH antagonist) relative to a measurement of the patient's triglyceride level obtained prior to the treatment period. In some embodiments, the patient does not exhibit an increase in serum triglyceride level of greater than 50% after 42 weeks of treatment with the GnRH antagonist (e.g., the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 42 weeks of treatment with the GnRH antagonist) relative to a measurement of the patient's triglyceride level obtained prior to the treatment period. In some embodiments, the patient does not exhibit an increase in serum triglyceride level of greater than 50% after 48 weeks of treatment with the GnRH antagonist (e.g., the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 48 weeks of treatment with the GnRH antagonist) relative to a measurement of the patient's triglyceride level obtained prior to the treatment period. In some embodiments, the patient does not exhibit an increase in serum triglyceride level of greater than 50% after 52 weeks of treatment with the GnRH antagonist (e.g., the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% after 52 weeks of treatment with the GnRH antagonist) relative to a measurement of the patient's triglyceride level obtained prior to the treatment period.

In some embodiments of any of the foregoing aspects (A1-A42) or embodiments of the disclosure, the method includes monitoring the patient's serum triglyceride level during the treatment period, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period). In some embodiments, the method includes monitoring the patient's serum triglyceride level after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period). In some embodiments, the method includes monitoring the patient's serum triglyceride level after 6 weeks of treatment with the GnRH antagonist, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period). In some embodiments, the method includes monitoring the patient's serum triglyceride level after 12 weeks of treatment with the GnRH antagonist, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period). In some embodiments, the method includes monitoring the patient's serum triglyceride level after 18 weeks of treatment with the GnRH antagonist, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period). In some embodiments, the method includes monitoring the patient's serum triglyceride level after 24 weeks of treatment with the GnRH antagonist, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period). In some embodiments, the method includes monitoring the patient's serum triglyceride level after 30 weeks of treatment with the GnRH antagonist, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period). In some embodiments, the method includes monitoring the patient's serum triglyceride level after 36 weeks of treatment with the GnRH antagonist, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period). In some embodiments, the method includes monitoring the patient's serum triglyceride level after 38 weeks of treatment with the GnRH antagonist, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period). In some embodiments, the method includes monitoring the patient's serum triglyceride level after 42 weeks of treatment with the GnRH antagonist, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period). In some embodiments, the method includes monitoring the patient's serum triglyceride level after 48 weeks of treatment with the GnRH antagonist, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period). In some embodiments, the method includes monitoring the patient's serum triglyceride level after 52 weeks of treatment with the GnRH antagonist, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period).

In some embodiments of any of the foregoing aspects (A1-A42) or embodiments of the disclosure, the patient does not exhibit an increase in serum triglyceride level to a level greater than 200 mg/dl at the end of the treatment period. In some embodiments, the patient does not exhibit an increase in serum triglyceride level to a level greater than 200 mg/dl after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist. In some embodiments, the patient does not exhibit an increase in serum triglyceride level to a level greater than 200 mg/dl after 6 weeks of treatment with the GnRH antagonist. In some embodiments, the patient does not exhibit an increase in serum triglyceride level to a level greater than 200 mg/dl after 12 weeks of treatment with the GnRH antagonist. In some embodiments, the patient does not exhibit an increase in serum triglyceride level to a level greater than 200 mg/dl after 18 weeks of treatment with the GnRH antagonist. In some embodiments, the patient does not exhibit an increase in serum triglyceride level to a level greater than 200 mg/dl after 24 weeks of treatment with the GnRH antagonist. In some embodiments, the patient does not exhibit an increase in serum triglyceride level to a level greater than 200 mg/dl after 30 weeks of treatment with the GnRH antagonist. In some embodiments, the patient does not exhibit an increase in serum triglyceride level to a level greater than 200 mg/dl after 36 weeks of treatment with the GnRH antagonist. In some embodiments, the patient does not exhibit an increase in serum triglyceride level to a level greater than 200 mg/dl after 38 weeks of treatment with the GnRH antagonist. In some embodiments, the patient does not exhibit an increase in serum triglyceride level to a level greater than 200 mg/dl after 42 weeks of treatment with the GnRH antagonist. In some embodiments, the patient does not exhibit an increase in serum triglyceride level to a level greater than 200 mg/dl after 48 weeks of treatment with the GnRH antagonist. In some embodiments, the patient does not exhibit an increase in serum triglyceride level to a level greater than 200 mg/dl after 52 weeks of treatment with the GnRH antagonist.

In some embodiments of any of the foregoing aspects (A1-A42) or embodiments of the disclosure, the method includes monitoring the patient's serum triglyceride level during the treatment period, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period). In some embodiments, the method includes monitoring the patient's serum triglyceride level after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period). In some embodiments, the method includes monitoring the patient's serum triglyceride level after 6 weeks of treatment with the GnRH antagonist, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period). In some embodiments, the method includes monitoring the patient's serum triglyceride level after 12 weeks of treatment with the GnRH antagonist, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period). In some embodiments, the method includes monitoring the patient's serum triglyceride level after 18 weeks of treatment with the GnRH antagonist, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period). In some embodiments, the method includes monitoring the patient's serum triglyceride level after 24 weeks of treatment with the GnRH antagonist, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period). In some embodiments, the method includes monitoring the patient's serum triglyceride level after 30 weeks of treatment with the GnRH antagonist, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period). In some embodiments, the method includes monitoring the patient's serum triglyceride level after 36 weeks of treatment with the GnRH antagonist, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period). In some embodiments, the method includes monitoring the patient's serum triglyceride level after 38 weeks of treatment with the GnRH antagonist, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period). In some embodiments, the method includes monitoring the patient's serum triglyceride level after 42 weeks of treatment with the GnRH antagonist, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period). In some embodiments, the method includes monitoring the patient's serum triglyceride level after 48 weeks of treatment with the GnRH antagonist, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period). In some embodiments, the method includes monitoring the patient's serum triglyceride level after 52 weeks of treatment with the GnRH antagonist, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period).

In some embodiments of any of the foregoing aspects (A1-A42) or embodiments of the disclosure, the patient does not exhibit an increase in serum triglyceride level from a level less than 300 mg/dl to a level greater than 500 mg/dl at the end of the treatment period. In some embodiments, the patient does not exhibit an increase in serum triglyceride level from a level less than 300 mg/dl to a level greater than 500 mg/dl after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist. In some embodiments, the patient does not exhibit an increase in serum triglyceride level from a level less than 300 mg/dl to a level greater than 500 mg/dl after 6 weeks of treatment with the GnRH antagonist. In some embodiments, the patient does not exhibit an increase in serum triglyceride level from a level less than 300 mg/dl to a level greater than 500 mg/dl after 12 weeks of treatment with the GnRH antagonist. In some embodiments, the patient does not exhibit an increase in serum triglyceride level from a level less than 300 mg/dl to a level greater than 500 mg/dl after 18 weeks of treatment with the GnRH antagonist. In some embodiments, the patient does not exhibit an increase in serum triglyceride level from a level less than 300 mg/dl to a level greater than 500 mg/dl after 24 weeks of treatment with the GnRH antagonist. In some embodiments, the patient does not exhibit an increase in serum triglyceride level from a level less than 300 mg/dl to a level greater than 500 mg/dl after 30 weeks of treatment with the GnRH antagonist. In some embodiments, the patient does not exhibit an increase in serum triglyceride level from a level less than 300 mg/dl to a level greater than 500 mg/dl after 36 weeks of treatment with the GnRH antagonist. In some embodiments, the patient does not exhibit an increase in serum triglyceride level from a level less than 300 mg/dl to a level greater than 500 mg/dl after 38 weeks of treatment with the GnRH antagonist. In some embodiments, the patient does not exhibit an increase in serum triglyceride level from a level less than 300 mg/dl to a level greater than 500 mg/dl after 42 weeks of treatment with the GnRH antagonist. In some embodiments, the patient does not exhibit an increase in serum triglyceride level from a level less than 300 mg/dl to a level greater than 500 mg/dl after 48 weeks of treatment with the GnRH antagonist. In some embodiments, the patient does not exhibit an increase in serum triglyceride level from a level less than 300 mg/dl to a level greater than 500 mg/dl after 52 weeks of treatment with the GnRH antagonist.

In some embodiments of the disclosure, the patient's total cholesterol level, low-density lipoprotein-cholesterol level, high-density lipoprotein-cholesterol level, serum triglyceride level, and/or ratio of low-density lipoprotein-cholesterol to high-density lipoprotein-cholesterol is assessed in a sample obtained from the patient, such as a sample of blood obtained from the patient. For example, in some embodiments, the measurement of the patient's total cholesterol level, low-density lipoprotein-cholesterol level, high-density lipoprotein-cholesterol level, serum triglyceride level, and/or ratio of low-density lipoprotein-cholesterol to high-density lipoprotein-cholesterol obtained prior to, or during, the treatment period is obtained by assessing the patient's total cholesterol level, low-density lipoprotein-cholesterol level, high-density lipoprotein-cholesterol level, serum triglyceride level, and/or ratio of low-density lipoprotein-cholesterol to high-density lipoprotein-cholesterol in a sample (e.g., a blood sample) obtained from the patient prior to, or during, the treatment period. In some embodiments, the measurement of the patient's total cholesterol level, low-density lipoprotein-cholesterol level, high-density lipoprotein-cholesterol level, serum triglyceride level, and/or ratio of low-density lipoprotein-cholesterol to high-density lipoprotein-cholesterol obtained after a period of time during the treatment period (e.g., a period of from about 6 weeks to about 52 weeks during the treatment period) is obtained by assessing the patient's total cholesterol level, low-density lipoprotein-cholesterol level, high-density lipoprotein-cholesterol level, serum triglyceride level, and/or ratio of low-density lipoprotein-cholesterol to high-density lipoprotein-cholesterol in a sample (e.g., a blood sample) obtained from the patient after a period of time during the treatment period (e.g., a period of from about 6 weeks to about 52 weeks during the treatment period).

In another aspect (“A43”), the disclosure features a kit containing a GnRH antagonist, such as a GnRH antagonist of any of the above aspects (A1-A42) or embodiments of the disclosure. The kit may further contain a package insert, such as a package insert instructing a user of the kit to administer the GnRH antagonist to a patient in accordance with the method of any one of the foregoing aspects (A1-A42) or embodiments of the disclosure. In some embodiments, the GnRH antagonist contained within the kit is a compound represented by formula (I)

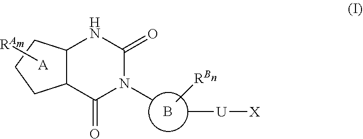

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof.

In some embodiments of formula (I), the ring A is a thiophene ring represented by formula (IIa)

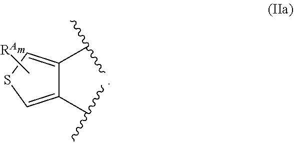

In some embodiments of formula (I) or (IIa), m is 1.

In some embodiments of formula (I) or (IIa), the ring A is an optionally substituted thiophene ring represented by formula (IIb)

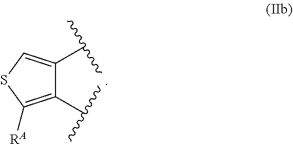

In some embodiments of formula (I), (IIa), or (IIb), each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

In some embodiments of formula (I), (IIa), or (IIb), each R^{A} is COOH or pharmaceutically acceptable salt thereof.

In some embodiments of formula (I), (IIa), or (IIb), the ring B is an optionally substituted benzene ring, pyridine ring, or thiophene ring.

In some embodiments of formula (I), (IIa), or (IIb), the ring B is represented by a formula selected from the group consisting of:

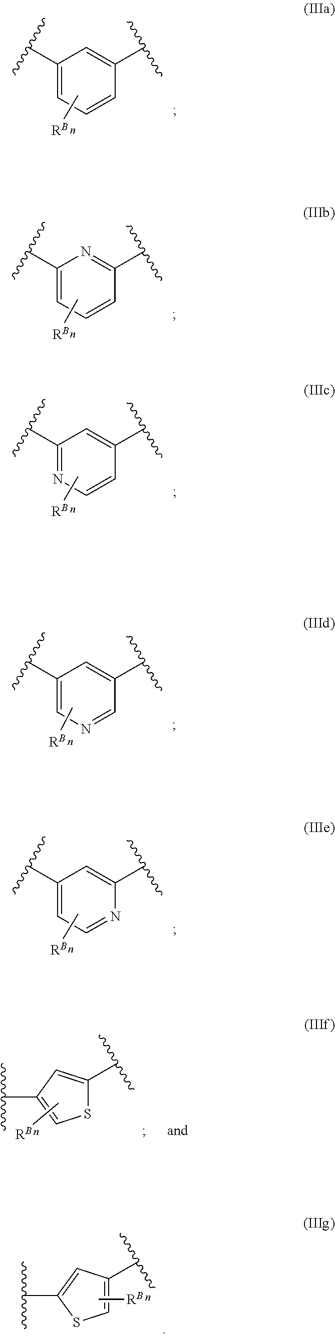

In some embodiments of formula (I), (IIa), (IIb), or any one of (IIIa)-(IIIg), n is 2. In some embodiments of formula (I), (IIa), (IIb), or any one of (IIIa)-(IIIg), the ring B is represented by a formula selected from the group consisting of:

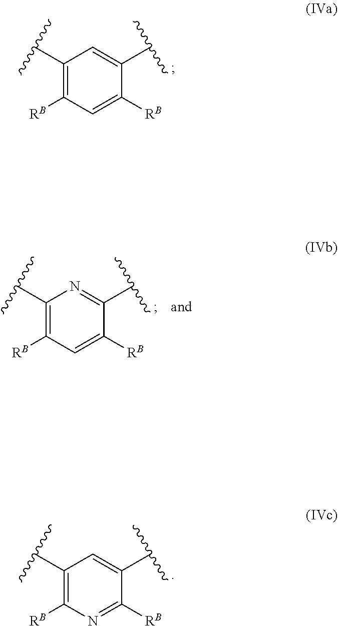

In some embodiments of formula (I), (IIa), (IIb), any one of (IIIa)-(IIIg), or any one of (IVa)-(IVc), each R^{B} is independently a halogen atom, an optionally substituted lower alkyl group, or OW⁴, wherein each W⁴ is independently a hydrogen atom or an optionally substituted lower alkyl group.

In some embodiments of formula (I), (IIa), (IIb), any one of (IIIa)-(IIIg), or any one of (IVa)-(IVc), each R^{B} is independently a fluorine atom, chlorine atom, bromine atom, methyl group, or methoxy group.

In some embodiments of formula (I), (IIa), (IIb), any one of (IIIa)-(IIIg), or any one of (IVa)-(IVc), U is a single bond.

In some embodiments of formula (I), (IIa), (IIb), any one of (IIIa)-(IIIg), or any one of (IVa)-(IVc), X is a group represented by —O-L-Y.

In some embodiments of formula (I), (IIa), (IIb), any one of (IIIa)-(IIIg), or any one of (IVa)-(IVc), L is a methylene group.

In some embodiments of formula (I), (IIa), (IIb), any one of (IIIa)-(IIIg), or any one of (IVa)-(IVc), Y is an optionally substituted benzene ring represented by formula (V)

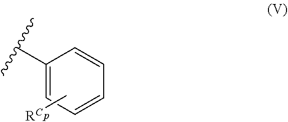

wherein each R^{C} is independently a halogen atom, an optionally substituted lower alkyl group, or OW⁹, wherein each W⁹ is independently a hydrogen atom or an optionally substituted lower alkyl group; and

p is an integer from 0 to 3.

In some embodiments of formula (I), (IIa), (IIb), any one of (IIIa)-(IIIg), any one of (IVa)-(IVc), or (V), Y is a substituted benzene ring represented by formula (Va)

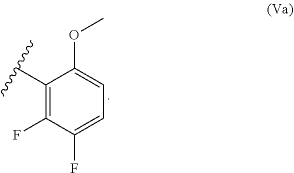 [SMILES: *c1c(OC)ccc(F)c1F]

In some embodiments, the compound is represented by formula (Ia)

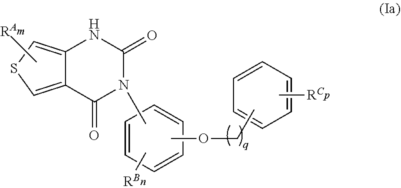

wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

q is an integer from 0 to 3;

each R^{C} is independently a halogen atom, an optionally substituted lower alkyl group, or OW⁹, wherein each W⁹ is independently a hydrogen atom or an optionally substituted lower alkyl group; and

p is an integer from 0 to 3;

or a pharmaceutically acceptable salt thereof.

In some embodiments, the compound is represented by formula (Ib)

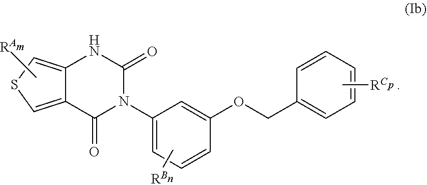

In some embodiments, the compound is represented by formula (Ic)

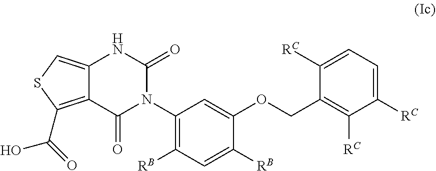

or a pharmaceutically acceptable salt thereof.

In some embodiments, the compound is 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid, represented by formula (VI)

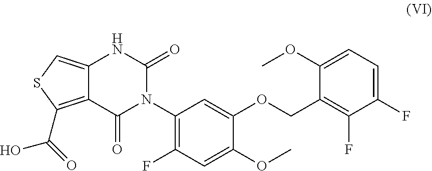 [SMILES: COc1cc(F)c(-n2c(=O)[nH]c3csc(C(=O)O)c3c2=O)cc1OCc1c(OC)ccc(F)c1F]

or a pharmaceutically acceptable salt thereof.

In some embodiments, the compound is the choline salt of the compound represented by formula (VI), choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate.

In some embodiments, the choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate is in a crystalline state.

In some embodiments, the choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate exhibits characteristic X-ray powder diffraction (XRPD) peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ.

In some embodiments, the choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm.

In some embodiments, the choline 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate exhibits ¹⁹F solid-state NMR peaks centered at about −151.8 ppm, −145.2 ppm, and −131.6 ppm.

In some embodiments, the GnRH antagonist contained within the kit is a compound represented by formula (VII)

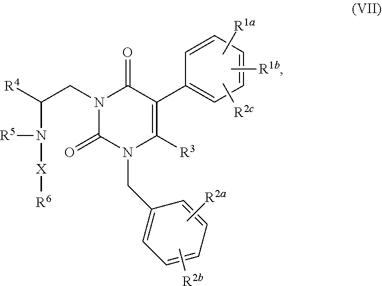

wherein R₁ₐ, R_{1b} and R_{1c} are the same or different and are each independently hydrogen, halogen, C₁₋₄alkyl, hydroxy or alkoxy, or R₁ₐ and R_{1b} taken together form —OCH₂O— or —OCH₂CH₂—;

R₂ₐ and R_{2b} are the same or different and are each independently hydrogen, halogen, trifluoromethyl, cyano or —SO₂CH₃;

R₃ is hydrogen or methyl;

R₄ is phenyl or C₃₋₇alkyl;

R₅ is hydrogen or C₁₋₄alkyl;

R₆ is —COOH or an acid isostere; and

X is C₁₋₆alkanediyl optionally substituted with from 1 to 3 C₁₋₆alkyl groups;

or a pharmaceutically acceptable salt thereof.

In some embodiments, the GnRH antagonist is a compound represented by formula (VIII)

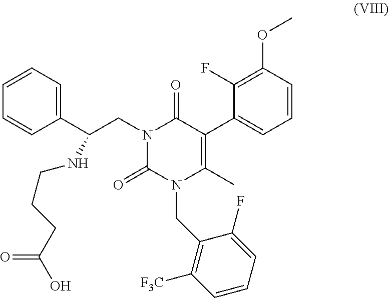 [SMILES: COc1cccc(-c2c(C)n(Cc3c(F)cccc3C(F)(F)F)c(=O)n(C[C@H](NCCCC(=O)O)c3ccccc3)c2=O)c1F]

or a pharmaceutically acceptable salt thereof. In some embodiments, the GnRH antagonist is the sodium salt of the compound represented by formula (VIII), which is represented by formula (IX), below.

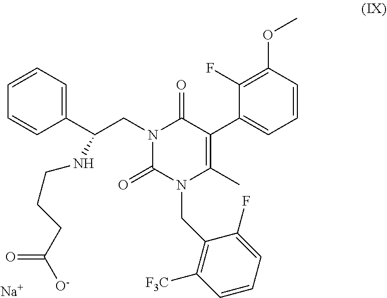 [SMILES: [Na+] ; COc1cccc(-c2c(C)n(Cc3c(F)cccc3C(F)(F)F)c(=O)n(C[C@H](NCCCC(=O)[O-])c3ccccc3)c2=O)c1F]

In some embodiments, the GnRH antagonist contained within the kit is a compound represented by formula (X)

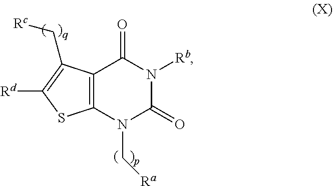

wherein R^{a} is a hydrogen atom, an optionally substituted aryl group (such as an aryl group that may have 1 to 5 substituents selected from halogen, nitro, cyano, amino, a carboxyl group that may be esterified or amidated, an alkylenedioxy, alkyl, alkoxy, alkylthio, alkylsulfinyl, and alkylsulfonyl), an optionally substituted cycloalkyl group, or an optionally substituted heterocyclic group;

R^{b} is an optionally substituted nitrogen-containing heterocyclic group;

R^{c} is an optionally substituted amino group;

R^{d} is an optionally substituted aryl group;

p is an integer from 0 to 3; and

q is an integer from 0 to 3;

or a pharmaceutically acceptable salt thereof.

In some embodiments, the GnRH antagonist is a compound represented by formula (XI)

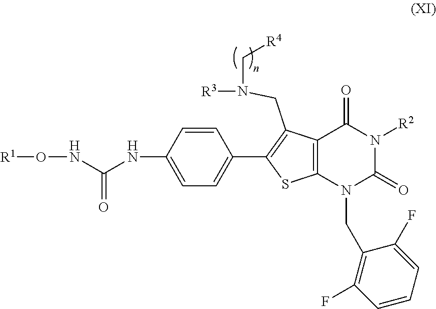

wherein R¹ is C₁₋₄alkyl;

R² is (1) a C₁₋₆alkyl which may have a substituent selected from the group consisting of (1′) a hydroxy group, (2′) a C₁₋₄alkoxy, (3′) a C₁₋₄alkoxy-carbonyl, (4′) a di-C₁₋₄alkyl-carbamoyl, (5′) a 5- to 7-membered nitrogen-containing heterocyclic group, (6′) a C₁₋₄alkyl-carbonyl and (7′) a halogen, (2) a C₃₋₈ cycloalkyl which may have (1′) a hydroxy group or (2′) a mono-C₁₋₄alkyl-carbonylamino, (3) a 5- to 7-membered nitrogen-containing heterocyclic group which may have a substituent selected from the group consisting of (1′) a halogen, (2′) a hydroxy group, (3′) a C₁₋₄alkyl and (4′) a C₁₋₄alkoxy, (4) a phenyl which may have a substituent selected from the group consisting of (1′) a halogen, (2′) a C₁₋₄alkoxy-C₁₋₄alkyl, (3′) a mono-C₁₋₄alkyl-carbamoyl-C₁₋₄alkyl, (4′) a C₁₋₄alkoxy and (5′) a mono-C₁₋₄alkylcarbamoyl-C₁₋₄alkoxy, or (5) a C₁₋₄alkoxy;

R³ is C₁₋₄alkyl;

R⁴ is (1) hydrogen, (2) C₁₋₄alkoxy, (3) C₆₋₁₀aryl, (4) N—C₁₋₄alkyl-N—C₁₋₄alkylsulfonylamino, (5) hydroxyl, or (6) a 5- to 7-membered nitrogen-containing heterocyclic group which may have a substituent selected from the group consisting of (1′) oxo, (2′) a C₁₋₄alkyl, (3′) a hydroxy-C₁₋₄alkyl, (4′) a C₁₋₄alkoxy-carbonyl, (5′) a mono-C₁₋₄alkyl-carbamoyl and (6′) a C₁₋₄alkylsulfonyl; and

n is an integer from 1 to 4;

optionally provided that when R² is a phenyl which may have a substituent, R⁴ is a 5- to 7-membered nitrogen-containing heterocyclic group which may have a substituent selected from the group consisting of (1) oxo, (2) hydroxy-C₁₋₄alkyl, (3) C₁₋₄alkoxy-carbonyl, (4) mono-C₁₋₄alkyl-carbamoyl and (5) C₁₋₄alkylsulfonyl;

or a pharmaceutically acceptable salt thereof. In some embodiments, the GnRH antagonist is a compound represented by formula (XII), below.

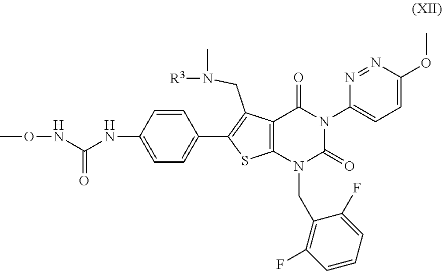

In some embodiments, the GnRH antagonist contained within the kit is a compound represented by formula (XIII)

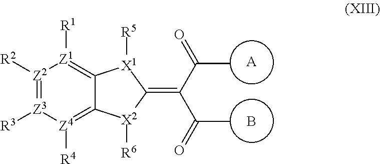

wherein R¹, R², R³ and R⁴ are the same or different, and are each independently selected from hydrogen, nitro, cyano, halogen, an optionally substituted hydrocarbon group, an optionally substituted heterocyclic group, hydroxy, alkoxy, carboxy, optionally substituted acyl-O—, optionally substituted acyl, a substituent —S(O)n₁₀₁- (wherein n₁₀₁ is an integer of 0 to 2), H—S(O)n₁₀₁-, optionally substituted carbamoyl, optionally substituted sulfamoyl, optionally substituted amino, and two adjacent groups selected from the group of R¹, R², R³ and R⁴ may combine to form an aryl or a carbocyclic (e.g., cycloalkenyl) group;

R⁵ and R⁶ are the same or different and are each independently selected from hydrogen, halogen, optionally substituted hydrocarbon, and optionally substituted amino,

X¹ and X² are the same or different and are each independently selected from N, S and O;

A and B are the same or different and are each independently selected from optionally substituted aryl and optionally substituted heterocyclyl, and

Z¹, Z², Z³ and Z⁴ are each independently selected from C and N; optionally provided that 1) when X¹ and X² each is S or O, one or both of the corresponding R⁵ and R⁶ are absent; and/or 2) when one to four of Z¹, Z², Z³ and/or Z⁴ are N, the corresponding R¹, R², R³ and/or R⁴ are absent;

or a pharmaceutically acceptable salt thereof. In some embodiments, the GnRH antagonist is a compound represented by formula (XIV), below.

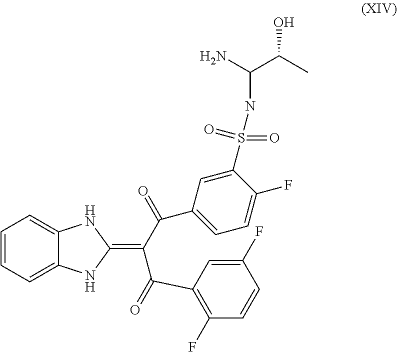 [SMILES: C[C@@H](O)C(N)NS(=O)(=O)c1cc(C(=O)C(C(=O)c2cc(F)ccc2F)=C2Nc3ccccc3N2)ccc1F]

In some embodiments, the GnRH antagonist contained within the kit is SKI2670 or BAY-784, or a variant or derivative thereof.

### Definitions

As used herein, the term “about” refers to a value that is within 10% above or below the value being described. For instance, a value of “about 5 mg” refers to a quantity that is from 4.5 mg to 5.5 mg.

As used herein, the term “abnormal uterine bleeding” refers to uterine blood loss that occurs either at an inappropriate time during a patient's menstrual cycle or in an amount that exceeds typical menstrual blood loss, such as “heavy menstrual blood loss” and “menorrhagia,” which refer to menstrual blood loss of 80 ml or more (e.g., 80 ml, 90 ml, 100 ml, 110 ml, 120 ml, 130 ml, 140 ml, 150 ml, 160 ml, 170 ml, 180 ml, 190 ml, 200 ml, or more) per menstrual cycle (The Menorrhagia Research Group. Quantification of menstrual blood loss. The Obstetrician & Gynaecologist 6:88-92 (2004)).

As used herein, the term “add-back therapy” refers to the administration of estrogen during a treatment regimen, such as treatment with a GnRH antagonist (e.g., 3-[2-fluoro-5-(2,3-difluoro-6-methoxybenzyloxy)-4-methoxyphenyl]-2,4-dioxo-1,2,3,4-tetrahydrothieno[3,4d]pyrimidine-5-carboxylic acid or a pharmaceutically acceptable salt thereof, such as the choline salt thereof, as described herein), so as to counteract side effects that may otherwise be associated with excessive suppression of estradiol. Such side effects may include, for example, a reduction in bone mineral density (BMD). A patient's BMD may be assessed by dual energy X-ray absorptiometry, for instance, in the spine or femur of the patient. Add-back therapy may be administered to a patient according to the methods described herein so as to mitigate a reduction in BMD caused by the administration of a GnRH antagonist. For instance, add-back therapy may be administered to a patient undergoing GnRH antagonist therapy such that the patient does not exhibit a reduction in BMD of greater than 5% (e.g., no greater than 5%, 4%, 3%, 2%, 1%, 0.9%, 0.8%, 0.7%, 0.6%, 0.5%, 0.4%, 0.3%, 0.2%, 0.1%, or less). Add-back therapy may include estrogen in the form of β17-estradiol, ethinyl estrogen, or a conjugated estrogen, such as a conjugated equine estrogen, and may further include one or more additional agents, such as a progestin (e.g., norethindrone or a compound that is metabolized in vivo to produce norethindrone, such as an ester of norethindrone that is de-esterified in vivo to produce norethindrone, for instance, norethindrone acetate, among other progestins such as progesterone, norgestimate, medroxyprogesterone, and drospirenone). Add-back therapy may be formulated for oral administration, such as in the form of a tablet, capsule, gel cap, powder, liquid solution, or liquid suspension. Add-back therapy may feature a co-formulation containing estrogen (e.g., in the form of β17-estradiol) and an additional agent such as a progestin (e.g., norethindrone or a compound that is metabolized in vivo to produce norethindrone, such as an ester of norethindrone that is de-esterified in vivo to produce norethindrone, for instance, norethindrone acetate). For instance, add-back therapy may be administered to a patient in the form of a single tablet, capsule, gel cap, powder, liquid solution, or liquid suspension that contains both estrogen (e.g., in the form of β17-estradiol) and a progestin (e.g., norethindrone or a compound that is metabolized in vivo to produce norethindrone, such as an ester of norethindrone that is de-esterified in vivo to produce norethindrone, for instance, norethindrone acetate). In some embodiments, add-back therapy is administered as a fixed dose combination containing a GnRH antagonist, estrogen, and one or more additional agents, such as a progestin, in a single pharmaceutical composition. For instance, add-back therapy may be administered as a fixed dose combination of a GnRH antagonist, estrogen (e.g., in the form of E2) and a progestin (e.g., norethindrone or a compound that is metabolized in vivo to produce norethindrone, such as an ester of norethindrone that is de-esterified in vivo to produce norethindrone, for instance, norethindrone acetate) in the form of a single pharmaceutical composition, such as a single tablet, capsule, gel cap, powder, liquid solution, or liquid suspension.

As used herein, a compound, such as a GnRH antagonist, estrogen, or progestin, among others, that is “administered” to a patient, such as a patient having an estrogen-dependent disease described herein, may be administered in an electrostatically neutral and/or nonionized form (e.g., in the form of a neutral carboxylic acid, a neutral amine, and the like) and/or in the form of a pharmaceutically acceptable salt, particularly if the compound contains a substituent that readily ionizes at physiological pH. For example, a compound containing a carboxylic acid substituent may be administered to a patient (e.g., a patient suffering from an estrogen-dependent disease described herein) in the form of the neutral, uncharged carboxylic acid and/or in the form of a carboxylate salt containing a pharmaceutically acceptable cation. Similarly, a compound containing an amine substituent may be administered to the patient in the form of the neutral, uncharged amine and/or in the form of an ammonium salt containing a pharmaceutically acceptable anion.

For example, as used herein, a GnRH antagonist of the disclosure, such as an optionally substituted thieno[3,4d]pyrimidine compound containing a carboxylic acid substituent, may be “administered” to a patient in the form of the neutral, uncharged carboxylic acid and/or in the form of a pharmaceutically acceptable salt (e.g., a salt containing the corresponding carboxylate anion and a pharmaceutically acceptable cation). Accordingly, as used herein, a GnRH antagonist of the formula 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid may be “administered” to a patient in the form of the neutral, uncharged carboxylic acid and/or in the form of a pharmaceutically acceptable salt (e.g., a salt containing the corresponding 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate anion and a pharmaceutically acceptable cation). For example, a GnRH antagonist of the formula 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid may be “administered” to a patient in the form of the neutral, uncharged carboxylic acid and/or in the form of a choline salt (i.e., a salt containing the corresponding 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate anion and a choline cation).

For example, as used herein, a GnRH antagonist of the disclosure, such as an optionally substituted 3-aminoalkyl pyrimidine-2,4(1H,3H)-dione compound containing a carboxylic acid substituent, may be “administered” to a patient in the form of the neutral, uncharged carboxylic acid and/or in the form of a pharmaceutically acceptable salt (e.g., a salt containing the corresponding carboxylate anion and a pharmaceutically acceptable cation). Accordingly, as used herein, a GnRH antagonist of the formula 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoic acid may be “administered” to a patient in the form of the neutral, uncharged carboxylic acid and/or in the form of a pharmaceutically acceptable salt (e.g., a salt containing the corresponding 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate anion and a pharmaceutically acceptable cation). For example, a GnRH antagonist of the formula 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoic acid may be “administered” to a patient in the form of the neutral, uncharged carboxylic acid and/or in the form of a sodium salt (i.e., a salt containing the corresponding 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate anion and a sodium cation).

For example, as used herein, a GnRH antagonist of the disclosure, such as an optionally substituted thieno[2,3d]pyrimidine compound containing an amine substituent, may be “administered” to a patient in the form of the neutral, uncharged amine and/or in the form of a pharmaceutically acceptable salt (e.g., a salt containing the corresponding ammonium cation and a pharmaceutically acceptable anion). Accordingly, as used herein, a GnRH antagonist of the formula N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea may be “administered” to a patient in the form of the neutral, uncharged amine and/or in the form of a pharmaceutically acceptable salt (e.g., a salt containing the corresponding, protonated N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea cation and a pharmaceutically acceptable anion). For example, a GnRH antagonist of the formula N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea may be “administered” to a patient in the form of the neutral, uncharged amine and/or in the form of a chloride salt (i.e., a salt containing the corresponding, protonated N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea cation and a chloride anion).

As used herein, a compound, such as a GnRH antagonist, estrogen, or progestin, among others, that is “administered” to a patient (e.g., a patient having an estrogen-dependent disease described herein) in a recited amount (e.g., per dose, per day, per week, per month, etc.) may be administered to the patient in the recited amount of an electrostatically neutral and/or nonionized form of the compound or in an equivalent amount of a pharmaceutically acceptable salt of the compound. As used herein, an amount of a pharmaceutically acceptable salt of a compound that is “equivalent” to a recited amount of the compound is an amount of the pharmaceutically acceptable salt that contains the same molar quantity of the compound as that contained by the recited amount of the compound. One can readily calculate the amount of a pharmaceutically acceptable salt of a compound that is “equivalent” to a recited amount of the compound using standard stoichiometry calculations known in the art.

Accordingly, as used herein, a GnRH antagonist of the disclosure, such as an optionally substituted thieno[3,4d]pyrimidine compound containing a carboxylic acid substituent, that is “administered” to a patient in a recited amount (e.g., per dose, per day, per week, per month, etc.) may be administered to the patient in the recited amount of an electrostatically neutral and/or nonionized of the compound or in an equivalent amount of a pharmaceutically acceptable salt of the compound. For example, as an optionally substituted thieno[3,4d]pyrimidine compound containing a carboxylic acid substituent, such as 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid, that is “administered” to a patient in a recited amount may be administered to the patient in the recited amount of the neutral, uncharged form of the compound or in an equivalent amount of a pharmaceutically acceptable salt of the compound (e.g., a salt containing the corresponding 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate anion and a pharmaceutically acceptable cation, such as a choline cation). Accordingly, an optionally substituted thieno[3,4d]pyrimidine compound containing a carboxylic acid substituent, such as 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid, that is “administered” to a patient in a recited amount, such as a recited amount of from 25 mg to 400 mg (e.g., 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, or 400 mg) may be administered to the patient in the recited amount of the neutral, uncharged form of the compound or in an equivalent amount of a pharmaceutically acceptable salt of the compound (e.g., a salt containing the corresponding 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylate anion and a pharmaceutically acceptable cation, such as a choline cation.

Accordingly, as used herein, a GnRH antagonist of the disclosure, such as an optionally substituted 3-aminoalkyl pyrimidine-2,4(1H,3H)-dione compound containing a carboxylic acid substituent, that is “administered” to a patient in a recited amount (e.g., per dose, per day, per week, per month, etc.) may be administered to the patient in the recited amount of an electrostatically neutral and/or nonionized form of the compound or in an equivalent amount of a pharmaceutically acceptable salt of the compound. For example, as an optionally substituted 3-aminoalkyl pyrimidine-2,4(1H,3H)-dione compound containing a carboxylic acid substituent, such as 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoic acid, that is “administered” to a patient in a recited amount may be administered to the patient in the recited amount of the neutral, uncharged form of the compound or in an equivalent amount of a pharmaceutically acceptable salt of the compound (e.g., a salt containing the corresponding 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate anion and a pharmaceutically acceptable cation, such as a sodium cation). Accordingly, an optionally substituted 3-aminoalkyl pyrimidine-2,4(1H,3H)-dione compound containing a carboxylic acid substituent, such as 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoic acid, that is “administered” to a patient in a recited amount, such as a recited amount of from 50 mg to 650 mg (e.g., 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, or 650 mg) may be administered to the patient in the recited amount of the neutral, uncharged form of the compound or in an equivalent amount of a pharmaceutically acceptable salt of the compound (e.g., a salt containing the corresponding 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate anion and a pharmaceutically acceptable cation, such as a sodium cation.

Accordingly, as used herein, a GnRH antagonist of the disclosure, such as an optionally substituted thieno[2,3d]pyrimidine compound containing an amine substituent, that is “administered” to a patient in a recited amount (e.g., per dose, per day, per week, per month, etc.) may be administered to the patient in the recited amount of an electrostatically neutral and/or nonionized form of the compound or in an equivalent amount of a pharmaceutically acceptable salt of the compound. For example, as an optionally substituted thieno[2,3d]pyrimidine compound containing an amine substituent, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, that is “administered” to a patient in a recited amount may be administered to the patient in the recited amount of the neutral, uncharged form of the compound or in an equivalent amount of a pharmaceutically acceptable salt of the compound (e.g., a salt containing the corresponding, protonated N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea cation and a pharmaceutically acceptable anion, such as a chloride anion). Accordingly, an optionally substituted thieno[2,3d]pyrimidine compound containing an amine substituent, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, that is “administered” to a patient in a recited amount, such as a recited amount of from 10 mg to 60 mg (e.g., 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg) may be administered to the patient in the recited amount of the neutral, uncharged form of the compound or in an equivalent amount of a pharmaceutically acceptable salt of the compound (e.g., a salt containing the corresponding, protonated N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea cation and a pharmaceutically acceptable anion, such as a chloride anion.

As used herein, the term “affinity” refers to the strength of a binding interaction between two molecules, such as a ligand and a receptor. The term “Kᵢ”, as used herein, is intended to refer to the inhibition constant of an antagonist for a particular molecule of interest, and can be expressed as a molar concentration (M). Kᵢ values for antagonist-target interactions can be determined, e.g., using methods established in the art. Methods that can be used to determine the Kᵢ of an antagonist for a molecular target include competitive binding experiments, e.g., as described in U.S. Pat. No. 9,040,693. The term “K_{d}”, as used herein, is intended to refer to the dissociation constant, which can be obtained, e.g., from the ratio of the rate constant for the dissociation of the two molecules (k_{d}) to the rate constant for the association of the two molecules (kₐ) and is expressed as a molar concentration (M). K_{d} values for receptor-ligand interactions can be determined, e.g., using methods established in the art. Methods that can be used to determine the K_{d} of a receptor-ligand interaction include surface plasmon resonance, e.g., through the use of a biosensor system such as a BIACORE® system.

As used herein, the term “amenorrhea” refers to the absence or near absence of uterine blood loss in a female patient, such as a female human patient undergoing GnRH antagonist treatment according to a dosing regimen described herein. As such, amenorrhea is a clinical indicator of reduced menstrual blood loss, such as reduced menstrual blood loss in a patient suffering from an estrogen-dependent disease (e.g., uterine fibroids, endometriosis (such as rectovaginal endometriosis), and adenomyosis, among others described herein) and undergoing GnRH antagonist treatment according to a dosing regimen described herein.

As used herein, the terms “benefit” and “response” are used interchangeably in the context of a subject undergoing therapy for the treatment of an estrogen-dependent disease described herein. These terms refers to any clinical improvement in the subject's condition. For example, clinical benefits in the context of a subject administered a gonadotropin-releasing hormone (GnRH) antagonist for the treatment of uterine fibroids, one of the estrogen-dependent diseases described herein, include, without limitation, (i) a reduction in serum concentration of FSH, LH, and/or E2 following administration of the GnRH antagonist to the patient, (ii) a reduction in uterine bleeding following administration of the GnRH antagonist to the patient; (iii) achievement of amenorrhea following administration of the GnRH antagonist to the patient; (iv) a reduction in the volume of one or more uterine fibroids following administration of the GnRH antagonist to the patient, (v) a reduction in pelvic pain following administration of the GnRH antagonist to the patient; (vi) a reduction in dysmenorrhea following administration of the GnRH antagonist to the patient; (vii) a reduction in dyspareunia following administration of the GnRH antagonist to the patient; (viii) a reduction in dyschezia following administration of the GnRH antagonist to the patient; and (ix) an improvement in the patient's overall well-being as determined, for example, by an improvement in the patient's Endometriosis Health Profile questionnaire (EHP-30) score following administration of the GnRH antagonist to the patient and/or by way of a positive Patient Global Impression of Change (PGIC) score following administration of the GnRH antagonist to the patient.

Similarly, exemplary clinical benefits in the context of a subject administered a GnRH antagonist for the treatment of endometriosis (e.g., rectovaginal endometriosis) include, without limitation, (i) a reduction in serum concentration of FSH, LH, and/or E2 following administration of the GnRH antagonist to the patient, (ii) a reduction in the volume of one or more endometriosis nodes (e.g., one or more rectovaginal endometriosis nodes) following administration of the GnRH antagonist to the patient, (iii) a reduction in bowel involvement of one or more type III endometriosis nodes following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine bleeding following administration of the GnRH antagonist to the patient; (ix) achievement of amenorrhea following administration of the GnRH antagonist to the patient; and (x) an improvement in the patient's overall well-being as determined by an improvement in the patient's EHP-30 score following administration of the GnRH antagonist to the patient and/or by way of a positive PGIC score following administration of the GnRH antagonist to the patient.

Exemplary clinical benefits in the context of a subject administered a GnRH antagonist for the treatment of adenomyosis, another estrogen-dependent disease described herein, include, without limitation, (i) a reduction in serum concentration of FSH, LH, and/or E2 following administration of the GnRH antagonist to the patient, (ii) a reduction in uterine volume following administration of the GnRH antagonist to the patient, (iii) a reduction in the thickness of the anterior and/or posterior region of the uterine myometrium following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine tenderness following administration of the GnRH antagonist to the patient; (ix) a reduction in uterine bleeding following administration of the GnRH antagonist to the patient; (x) achievement of amenorrhea following administration of the GnRH antagonist to the patient; (xi) a reduction in the diameter of a junctional zone of adenomyosis following administration of the GnRH antagonist to the patient; and (xii) an improvement in the patient's overall well-being as determined by an improvement in the patient's EHP-30 score following administration of the GnRH antagonist to the patient and/or by way of a positive PGIC score following administration of the GnRH antagonist to the patient.

As used herein, the term “Biberoglu and Behrman scale” or “B&B scale” or a modification thereof, such as a “modified Biberoglu and Behrman scale” refers to a multi-point scale that can be used to indicate the severity of one or more symptoms experienced by patient suffering from an estrogen-dependent disease, such as endometriosis, among others. A B&B score can be assessed by verbally prompting the patient to indicate the degree of function or quality of life being experienced. A B&B score can be used, e.g., to assess the severity of such symptoms as dysmenorrhea, dyspareunia, chronic pelvic pain, pelvic tenderness, and induration, among others. Methods of determining a B&B score are described in detail, e.g., in Biberoglu and Behrman, Am. J. Obstet. Gynecol. 139:645 (1981). As used herein, the term “crystalline” or “crystalline form” means having a physical state that is a regular three-dimensional array of atoms, ions, molecules or molecular assemblies. Crystalline forms have lattice arrays of building blocks called asymmetric units that are arranged according to well-defined symmetries into unit cells that are repeated in three-dimensions. In contrast, the term “amorphous” or “amorphous form” refers to an unorganized (no orderly) structure. The physical state of a therapeutic compound may be determined by exemplary techniques such as x-ray diffraction, polarized light microscopy and/or differential scanning calorimetry.

As used herein, the term “dose” refers to the quantity of a therapeutic agent, such as a GnRH antagonist described herein, that is administered to a subject at a particular instant for the treatment of a disorder or condition, such as to treat or ameliorate one or more symptoms of an estrogen-dependent disease described herein (e.g., uterine fibroids, endometriosis, such as rectovaginal endometriosis, and/or uterine fibroids). A therapeutic agent as described herein may be administered in a single dose or in multiple doses over the course of a treatment period, as defined herein. In each case, the therapeutic agent may be administered using one or more “unit dosage forms” of the therapeutic agent, a term that refers to a one or more discrete compositions containing a therapeutic agent that collectively constitute a single dose of the agent. For instance, a single dose of 200 mg of a therapeutic agent may be administered using, e.g., two 100 mg unit dosage forms of the therapeutic agent. The unit dosage forms may be, for example, solid unit dosage forms, such as tablets or capsules, among others.

As used herein, the term “dual energy X-ray absorptiometry” (DEXA) refers to a spectroscopic method of measuring bone mineral density in a patient (e.g., a human patient) in which X-ray radiation of two distinct frequencies are transmitted towards a target bone of the patient. The absorption of the transmitted radiation can subsequently be correlated with a measure of the bone mineral density within the target bone. Methods of determining bone mineral density using DEXA are described in detail, e.g., in Mazess et al., American Journal of Clinical Nutrition 51:1106-1112 (1990).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “Endometriosis Health Profile-30” or “EHP-30” refers to a questionnaire that can be used to evaluate quality of life in patient suffering from an estrogen-dependent disease, such as endometriosis, among others. A score obtained from this questionnaire (i.e., an “EHP-30 score”) may provide an indication of the patient's degree of pain, feeling of control and powerlessness, emotional well-being, social support, and/or self-image. Exemplary methods that can be used to perform an EHP-30 questionnaire and procedures for interpreting the scores obtained therefrom are known in the art are described, e.g., in Renouvel et al., Journal de Gynécologie Obstétrique et Biologie de la Reproduction 38:404-410 (2009), the disclosure of which is incorporated herein by reference as it pertains to methods for conducting and evaluating an EHP-30 questionnaire.

As used herein, the term “estrogen-dependent disease” refers to a disease or condition that is exacerbated or caused by excessive, inappropriate, or unregulated estrogen (e.g., β17-estradiol) production and/or an aberrant physiological response to estrogen. Estrogen-dependent diseases include those exacerbated or caused by circulating β17-estradiol levels in excess of, for example, about 60 μg/ml. Examples of such diseases include uterine fibroids, endometriosis (e.g., rectovaginal endometriosis), and adenomyosis. Additional examples of estrogen-dependent diseases include, without limitation, benign prostatic hypertrophy, precocious puberty, premenstrual syndrome, polycystic ovary syndrome, hirsutism, short stature, sleep disorders, acne, baldness, and irritable bowel syndrome, among others.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “gonadotropin-releasing hormone antagonist” or “GnRH antagonist” refers to a compound that specifically binds the GnRH receptor and is capable of inhibiting receptor signaling, e.g., such that release of one or more gonadotropins (such as follicle-stimulating hormone and luteinizing hormone) is inhibited. GnRH antagonists for use with the compositions and methods described herein include thieno[3,4d]pyrimidine derivatives and variants, such as those described in U.S. Pat. No. 9,040,693, the disclosure of which is incorporated herein by reference in its entirety. Exemplary GnRH antagonists include 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid or a pharmaceutically acceptable salt thereof, such as the choline salt thereof, e.g., as described in U.S. Pat. No. 9,169,266, the disclosure of which is incorporated herein by reference in its entirety. Additional examples of GnRH antagonists that may be used in conjunction with the compositions and methods described herein include optionally substituted 3-aminoalkyl pyrimidine-2,4(1H,3H)-dione derivatives, such as sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate, also referred to as elagolix, or the carboxylic acid conjugate thereof, and related compounds described in U.S. Pat. No. 7,056,927, the disclosure of which is incorporated herein by reference in its entirety. Further examples of GnRH antagonists that may be used in conjunction with the compositions and methods described herein include optionally substituted thieno[2,3d]pyrimidine derivatives, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, also referred to as relugolix, or a pharmaceutically acceptable salt thereof, and related compounds described in U.S. Pat. No. 7,300,935, the disclosure of which is incorporated herein by reference in its entirety. Additional examples of GnRH antagonists that may be used in conjunction with the compositions and methods described herein include optionally substituted propane-1,3-dione derivatives, such as (2R)—N-{5-[3-(2,5-difluorophenyl)-2-(1,3-dihydro-2H-benzimidazol-2-ylidene)-3-oxopropanoyl]-2-fluorobenzene-1-sulfonyl}-2-hydroxypropanimidamide, also referred to as opigolix or ASP-1707, and related compounds described in U.S. Pat. No. 6,960,591, the disclosure of which is incorporated herein by reference in its entirety.

As used herein, the term “IC₅₀” refers to the concentration of a substance (antagonist) that reduces the efficacy of a reference agonist or the constitutive activity of a biological target by 50%, e.g., as measured in a competitive ligand binding assay. Exemplary competitive ligand binding assays include competitive radioligand binding assays, competitive enzyme-linked immunosorbant assays (ELISA), and fluorescence anisotropy-based assays, among others known in the art.

As used herein in the context of a GnRH antagonist and add-back therapy, the term “in combination with” refers to administration of the GnRH antagonist and add-back therapy agent(s) such that the later-administered of these substances is provided to the patient at a time when there is still a detectable concentration in the patient's blood of the earlier-administered of these substances. The GnRH antagonist and add-back therapy need not be administered at the exact same moment for these substances to be administered “in combination with” one another.

As used herein, for example, in the context of detecting and quantifying one or more of a patient's serum lipids, the term “level” refers to a concentration of the analyte of interest, such as a concentration of total cholesterol, low-density lipoprotein-cholesterol, high-density lipoprotein-cholesterol, or triglycerides, in vivo in the patient or in an ex vivo sample obtained from the patient. A level of an analyte of interest in a patient, such as a level of one or more of the foregoing lipids in a patient, may be assessed, for example, in a sample obtained from the patient, such as a sample of blood serum obtained from the patient. Similarly, as used herein, a “ratio” of a first analyte to a second analyte, such as a ratio of a patient's low-density lipoprotein to high-density lipoprotein, refers to a numerical ratio of the level of the first analyte (e.g., as assessed in a sample obtained from the patient, such as a blood serum sample) to a level of the second analyte (e.g., as assessed in a sample obtained from the patient, such as a blood serum sample). As used herein, a level of an analyte of interest or a ratio of one analyte of interest to another analyte of interest are said to increase “significantly” if, for example, the level or ratio increases by 50% or more (e.g., by 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, or more) relative, for example, to a previous measurement of the level of ratio. For example, a level of a patient's total cholesterol, low-density lipoprotein-cholesterol, high-density lipoprotein-cholesterol, or triglycerides is considered to increase significantly if the level of the patient's total cholesterol, low-density lipoprotein-cholesterol, high-density lipoprotein-cholesterol, or triglycerides, as the case may be, increases by 50% (e.g., by 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, or more) relative to a previous measurement of the patient's total cholesterol, low-density lipoprotein-cholesterol, high-density lipoprotein-cholesterol, or triglycerides, such as a measurement of the patient's total cholesterol, low-density lipoprotein-cholesterol, high-density lipoprotein-cholesterol, or triglycerides obtained prior to commencement of treatment with a therapeutic agent (e.g., a GnRH antagonist, such as a GnRH antagonist described herein).

As used herein in the context of one or more serum lipids, such as total cholesterol, low-density lipoprotein-cholesterol, high-density lipoprotein-cholesterol, or triglycerides, the term “monitor” means to conduct a test on a patient (e.g., a patient undergoing treatment for an estrogen-dependent disease described herein by administration of a GnRH antagonist) in order to determine the patient's level of the indicated serum lipid(s) and/or the patient's ratio of one serum lipid to another, as context dictates. The test may involve, for example, obtaining a sample from the patient, such as a sample of blood serum, and detecting the indicated serum lipid(s) in the sample ex vivo. In some embodiments of the present disclosure, a patient undergoing treatment for an estrogen-dependent disease described herein by periodic administration of a GnRH antagonist during a treatment period has their level of one or more serum lipids and/or their ratio of one serum lipid to another “monitored” during the treatment period. This language shall be understood to mean that the patient is (i) periodically administered the GnRH antagonist and then, after a certain duration of treatment, the patient is (ii) subjected to a test during the treatment period in order to determine the patient's level of the indicated serum lipid(s) and/or the patient's ratio of one serum lipid to another, as context dictates. The test may involve, for example, obtaining a sample from the patient, such as a sample of blood serum, and quantifying the indicated serum lipid(s) in the sample ex vivo. In some embodiments, the patient may then resume treatment with the GnRH antagonist for the remainder of the treatment period, for example, if the patient is determined to not exhibit a significant increase in the level of the indicated serum lipid(s) and/or the ratio of one serum lipid to another, as is described herein.

In some embodiments of the disclosure, one or more of a patient's serum lipid levels (such as total cholesterol, low-density lipoprotein-cholesterol, high-density lipoprotein-cholesterol, or triglycerides), and/or the patient's ratio of one serum lipid to another (such as the patient's ratio of low-density lipoprotein-cholesterol to high-density lipoprotein-cholesterol), is monitored during a treatment period in which the patient is administered (e.g., periodically administered) a GnRH antagonist, such as a GnRH antagonist described herein. In some embodiments, the one or more serum lipid levels, and/or the patient's ratio of one serum lipid to another, are monitored from about 1 day to about 1 year after commencement of treatment with the GnRH antagonist (e.g., from about 1 week to about 52 weeks after commencement of the treatment period, from about 2 weeks to about 52 weeks after commencement of the treatment period, from about 3 weeks to about 52 weeks after commencement of the treatment period, from about 4 weeks to about 52 weeks after commencement of the treatment period, from about 5 weeks to about 52 weeks after commencement of the treatment period, from about 6 weeks to about 52 weeks after commencement of the treatment period, from about 7 weeks to about 52 weeks after commencement of the treatment period, from about 8 weeks to about 52 weeks after commencement of the treatment period, from about 9 weeks to about 52 weeks after commencement of the treatment period, from about 10 weeks to about 52 weeks after commencement of the treatment period, from about 11 weeks to about 52 weeks after commencement of the treatment period, from about 12 weeks to about 52 weeks after commencement of the treatment period, from about 13 weeks to about 52 weeks after commencement of the treatment period, from about 14 weeks to about 52 weeks after commencement of the treatment period, from about 15 weeks to about 52 weeks after commencement of the treatment period, from about 16 weeks to about 52 weeks after commencement of the treatment period, from about 17 weeks to about 52 weeks after commencement of the treatment period, from about 18 weeks to about 52 weeks after commencement of the treatment period, from about 19 weeks to about 52 weeks after commencement of the treatment period, from about 20 weeks to about 52 weeks after commencement of the treatment period, from about 21 weeks to about 52 weeks after commencement of the treatment period, from about 22 weeks to about 52 weeks after commencement of the treatment period, from about 23 weeks to about 52 weeks after commencement of the treatment period, or from about 24 weeks to about 52 weeks after commencement of the treatment period, or more (e.g., about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks, or more, after commencement of the treatment period).

As used herein, the term “menstrual cycle” refers to a recurring cycle of physiological changes in females, such as human females, that is associated with reproductive fertility. While the cycle length may vary from woman to woman, 28 days is generally taken as representative of the average ovulatory cycle in human females.

As used herein, the term “Numerical Rating Score” (NRS) refers to a score within an 11-point numerical scale of 0-10 that indicates the degree of pain experienced by a patient, such as a patient having an estrogen-dependent disease described herein. For instance, a score of 0 may indicate the patient is experiencing no pain, while scores from 1-3 may indicate that the patient is experiencing mild pain. A score of from 4-6 may indicate that the patient is experiencing moderate pain, and a score of from 7-10 may indicate that the patient is experiencing severe pain. Typically, to determine a NRS score, the patient is asked to indicate the level of pain currently being experienced, as well as the pain experienced at its most intense and least intense occurrences. Methods for determining a NRS are described in detail, e.g., in McCaffery et al., Pain: Clinical Manual for Nursing Practice. Baltimore (1993), the disclosure of which is incorporated herein by reference as it pertains to methods for obtaining and evaluating an NRS.

As used herein, the term “pharmaceutical composition” means a mixture containing a therapeutic compound to be administered to a patient, such as a mammal, e.g., a human, in order to prevent, treat or control a particular disease or condition affecting the mammal, such as preterm labor or dysmenorrhea.

As used herein, the term “pharmaceutically acceptable” refers to those compounds, materials, compositions and/or dosage forms, which are suitable for contact with the tissues of a patient, such as a mammal (e.g., a human) without excessive toxicity, irritation, allergic response and other problem complications commensurate with a reasonable benefit/risk ratio.

As used herein in the context of administration of a therapeutic agent, the term “periodically” refers to administration of the agent two or more times over the course of a treatment period (e.g., two or more times daily, weekly, monthly, or yearly).

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a patient.

As used herein in the context of a reduction in bone mineral density loss, the term “significant” refers to a percentage of bone mineral density loss that exceeds a safe limit, as assessed, for example, by a physician of skill in the art. Examples of “significant” reductions in bone mineral density are reductions in excess of 5%. For example, as used herein, a patient (e.g., a patient suffering from an estrogen-dependent disease, such as uterine fibroids, endometriosis, adenomyosis, or rectovaginal endometriosis) is considered to exhibit a “significant” reduction in bone mineral density following a treatment period in which the patient is periodically administered a GnRH antagonist if the patient exhibits a loss in bone mineral density of greater than 5% at the end of the treatment period relative to a measurement of the patient's bone mineral density obtained prior to, or during, the treatment period. A patient is not considered to exhibit a “significant” reduction in bone mineral density following such a treatment period if the patient exhibits a reduction in bone mineral density of less than 5% at the end of the treatment period relative to a measurement of the patient's bone mineral density obtained prior to, or during, the treatment period, such as a reduction in bone mineral density of 5%, 4%, 3%, 2%, 1%, 0.9%, 0.8%, 0.7%, 0.6%, 0.5%, 0.4%, 0.3%, 0.2%, 0.1%, or less at the end of the treatment period relative to a measurement of the patient's bone mineral density obtained prior to, or during, the treatment period.

As used herein, the phrases “specifically binds” and “binds” refer to a binding reaction which is determinative of the presence of a particular protein in a heterogeneous population of proteins and other biological molecules that is recognized, e.g., by a ligand with particularity. A ligand (e.g., a protein, proteoglycan, or glycosaminoglycan) that specifically binds to a protein will bind to the protein, e.g., with a K_{D} of less than 100 nM. For example, a ligand that specifically binds to a protein may bind to the protein with a K_{D} of up to 100 nM (e.g., between 1 pM and 100 nM). A ligand that does not exhibit specific binding to a protein or a domain thereof may exhibit a K_{D} of greater than 100 nM (e.g., greater than 200 nM, 300 nM, 400 nM, 500 nM, 600 nm, 700 nM, 800 nM, 900 nM, 1 μM, 100 μM, 500 μM, or 1 mM) for that particular protein or domain thereof. A variety of assay formats may be used to determine the affinity of a ligand fora specific protein. For example, solid-phase ELISA assays are routinely used to identify ligands that specifically bind a target protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the terms “subject” and “patient” are used interchangeably and refer to an organism, such as a mammal (e.g., a human) that receives treatment for an estrogen-dependent disease described herein, such as uterine fibroids, endometriosis (e.g., rectovaginal endometriosis) and/or adenomyosis using the compositions and methods described herein and/or that is diagnosed as having one or more of these diseases in accordance with the methods described herein. Examples of patients include pre-menopausal female human patients. For instance, uterine fibroids patients in need of treatment in accordance with the compositions and methods described herein include those experiencing heavy menstrual bleeding (i.e., blood loss in excess of 80 ml per menstrual cycle). Examples of adenomyosis patients in need of treatment using a GnRH antagonist according to the methods described herein include, e.g., adenomyosis patients diagnosed as having a junctional-zone width of about 12 mm or more prior to administration of the GnRH antagonist to the patient (e.g., a junctional zone width of from about 12 mm to about 20 mm, from about 12 mm to about 19 mm, from about 12 mm to about 18 mm, from about 12 mm to about 17 mm, from about 12 mm to about 16 mm, from about 12 mm to about 15 mm, from about 12 mm to about 14 mm, or more, prior to administration of the GnRH antagonist to the patient). Examples of endometriosis patients (e.g., rectovaginal endometriosis patients) in need of treatment using a GnRH antagonist according to the methods described herein include, e.g., rectovaginal endometriosis patients exhibiting a rectal (type II) and/or vaginal (type III) endometriosis node of at least 2 cm prior to administration of the GnRH antagonist to the patient, such as a rectal (type II) and/or vaginal (type III) endometriosis node of from about 2 cm to about 10 cm, or more (e.g., a rectal (type II) and/or vaginal (type III) endometriosis node of from about 2 cm to about 9 cm, from about 2 cm to about 8 cm, from about 2 cm to about 7 cm, from about 2 cm to about 6 cm, from about 2 cm to about 5 cm, or from about 2 cm to about 4 cm, or more) prior to administration of the GnRH antagonist to the patient.

As used herein in the context of a GnRH antagonist, the term “therapeutically effective amount” refers to the quantity of a GnRH antagonist that, when administered to a patient (e.g., a patient suffering from an estrogen-dependent disease), is capable of promoting a reduction in endogenous ß17-estradiol levels to concentrations that are less likely to trigger the onset of, or sustain the progression of, an estrogen-dependent disease, such as a concentration of less than about 60 μg/ml in circulating blood. Exemplary therapeutically effective amounts of a GnRH antagonist include, e.g., from about 50 mg to about 200 mg of a compound represented by any one of formulas (I)-(VIa), among other dosage quantities described herein.

As used herein, the terms “treat” or “treatment” refer to therapeutic treatment, in which the object is to prevent or slow down (lessen) an undesired physiological change or disorder in a human patient, such as the progression of an estrogen-dependent disease described herein, including uterine fibroids, endometriosis (e.g., rectovaginal endometriosis) and adenomyosis, among others. Beneficial or desired clinical results include, but are not limited to, alleviation of symptoms, such as a reduction in pelvic pain, a reduction in dysmenorrhea, a reduction in dyspareunia, a reduction in dyschezia, and a reduction in uterine bleeding, among other desired benefits described herein. As a non-limiting example, a patient, such as a female human patient, suffering from uterine fibroids may considered to be treated using a GnRH antagonist described herein if the patient exhibits (i) a reduction in uterine blood loss (e.g., elimination of heavy menstrual blood loss) following administration of the GnRH antagonist to the patient; and/or (ii) induction of amenorrhea following administration of the GnRH antagonist to the patient.

Similarly, clinical indicators of successful treatment of a patient having endometriosis (e.g., rectovaginal endometriosis) using a GnRH antagonist described herein include (i) a reduction in the volume of one or more endometriosis nodes (e.g., rectovaginal endometriosis nodes) following administration of the GnRH antagonist to the patient; (ii) a reduction in bowel involvement of one or more type III endometriosis nodes following administration of the GnRH antagonist to the patient; (iii) a reduction in pelvic pain following administration of the GnRH antagonist to the patient; (iv) a reduction in dysmenorrhea following administration of the GnRH antagonist to the patient; (v) a reduction in dyspareunia following administration of the GnRH antagonist to the patient; (vi) a reduction in dyschezia following administration of the GnRH antagonist to the patient; (vii) a reduction in uterine tenderness following administration of the GnRH antagonist to the patient; (viii) a reduction in uterine bleeding following administration of the GnRH antagonist to the patient; (ix) achievement of amenorrhea following administration of the GnRH antagonist to the patient; and/or (x) an improvement in the patient's overall well-being as determined by an improvement in the patient's EHP-30 score following administration of the GnRH antagonist to the patient and/or the observation of a positive PGIC score following administration of the GnRH antagonist to the patient.

Clinical indicators of successful treatment of a patient having adenomyosis, another estrogen-dependent disease described herein, include, without limitation, (i) a reduction in uterine volume following administration of the GnRH antagonist to the patient; (ii) a reduction in the thickness of the anterior and/or posterior region of the uterine myometrium following administration of the GnRH antagonist to the patient; (iii) a reduction in pelvic pain following administration of the GnRH antagonist to the patient; (iv) a reduction in dysmenorrhea following administration of the GnRH antagonist to the patient; (v) a reduction in dyspareunia following administration of the GnRH antagonist to the patient; (vi) a reduction in dyschezia following administration of the GnRH antagonist to the patient; (vii) a reduction in uterine tenderness following administration of the GnRH antagonist to the patient; (viii) a reduction in uterine bleeding following administration of the GnRH antagonist to the patient; (ix) achievement of amenorrhea following administration of the GnRH antagonist to the patient; (x) a reduction in the diameter of a junctional zone of adenomyosis following administration of the GnRH antagonist to the patient; and/or (xi) an improvement in the patient's overall well-being as determined by an improvement in the patient's EHP-30 score following administration of the GnRH antagonist to the patient and/or the observation of a positive PGIC score following administration of the GnRH antagonist to the patient.

As used herein, the term “treatment period” refers to a duration of time over which a patient may be periodically administered a therapeutic agent, such as a GnRH antagonist described herein. Treatment periods as described herein may have a duration of, for example, several days, weeks, or months. For instance, a treatment period for administration of a thieno[3,4d]pyrimidine derivative, such as 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid or a pharmaceutically acceptable salt thereof, such as the choline salt thereof, may last for from about four weeks to about six months (e.g., from about 28 days to about 180 days, about 30 days to about 175 days, about 35 days to about 170 days, about 40 days to about 165 days, about 45 days to about 160 days, about 50 days to about 155 days, about 55 days to about 150 days, about 60 days to about 145 days, about 65 days to about 140 days, about 70 days to about 135 days, about 75 days, to about 130 days, about 80 days to about 125 days, about 85 days, to about 120 days, or about 90 days to about 115 days). In some embodiments, the GnRH antagonist is periodically administered to the patient over a treatment period of from about eight weeks to about sixteen weeks (e.g., from about 60 days to about 110 days, about 65 days to about 105 days, about 70 days to about 100 days, about 75 days to about 95 days, or about 80 days, to about 90 days). In some embodiments, the GnRH antagonist is periodically administered to the patient over a treatment period of about twelve weeks. In some embodiments, the GnRH antagonist is periodically administered to the patient over a treatment period of from about 20 weeks to about 30 weeks (e.g., from about 140 days to about 210 days, about 150 days to about 100 days, about 60 days to about 90 days, about 65 days to about 85 days, or about 68 days). In some embodiments, the GnRH antagonist is periodically administered to the patient over a treatment period of about 24 weeks. For the avoidance of doubt, the phrase “over the course of a treatment period” and “over a treatment period,” when used in the context of a therapeutic agent that is periodically administered to a patient, signify that the recited therapeutic agent (e.g., a GnRH antagonist described herein) is administered to the patient throughout the full duration of the treatment period at the indicated frequency.

As used herein, the term “Verbal Rating Score” (VRS) refers to a subjective multi-point scale used to indicate the level of pain being experienced by a patient undergoing therapy or that has previously undergone therapy for a disease or condition, such as an estrogen-dependent disease described herein. The VRS may be a five-point scale and can be assessed by prompting the patient with one or more questions in order to determine the level of pain currently being experienced by the patient. Methods for assessing a VRS are described in detail, e.g., in Jensen et al., Journal of Pain and Symptom Management 41:1073-1093 (2011), the disclosure of which is incorporated herein by reference as it pertains to methods for obtaining and evaluating a VRS.

As used herein, the term “aryl” refers to an unsaturated aromatic carbocyclic group of from 6 to 14 carbon atoms having a single ring (e.g., optionally substituted phenyl) or multiple condensed rings (e.g., optionally substituted naphthyl). Exemplary aryl groups include phenyl, naphthyl, phenanthrenyl, and the like.

As used herein, the term “cycloalkyl” refers to a monocyclic cycloalkyl group having from 3 to 8 carbon atoms, such as cyclopropyl, cyclobutyl, cyclopentyl, cyclohexyl, cycloheptyl, cyclooctyl, and the like.

As used herein, the term “halogen atom” refers to a fluorine atom, a chlorine atom, a bromine atom, or an iodine atom.

As used herein, the term “heteroaryl” refers to a monocyclic heteroaromatic, or a bicyclic or a tricyclic fused-ring heteroaromatic group. Exemplary heteroaryl groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “heterocycloalkyl” refers to a 3 to 8-membered heterocycloalkyl group having 1 or more heteroatoms, such as a nitrogen atom, an oxygen atom, a sulfur atom, and the like, and optionally having 1 or 2 oxo groups such as pyrrolidinyl, piperidinyl, oxopiperidinyl, morpholinyl, piperazinyl, oxopiperazinyl, thiomorpholinyl, azepanyl, diazepanyl, oxazepanyl, thiazepanyl, dioxothiazepanyl, azokanyl, tetrahydrofuranyl, tetrahydropyranyl, and the like.

As used herein, the terms “lower alkyl” and “C₁₋₆ alkyl” refer to an optionally branched alkyl moiety having from 1 to 6 carbon atoms, such as methyl, ethyl, propyl, isopropyl, butyl, isobutyl, sec-butyl, tert-butyl, pentyl, isopentyl, neopentyl, tert-pentyl, hexyl, and the like.

As used herein, the term “lower alkylene” refers to an optionally branched alkylene group having from 1 to 6 carbon atoms, such as methylene, ethylene, methylmethylene, trimethylene, dimethylmethylene, ethylmethylene, methylethylene, propylmethylene, isopropylmethylene, dimethylethylene, butylmethylene, ethylmethylmethylene, pentamethylene, diethylmethylene, dimethyltrimethylene, hexamethylene, diethylethylene and the like.

As used herein, the term “lower alkenyl” refers to an optionally branched alkenyl moiety having from 2 to 6 carbon atoms, such as vinyl, allyl, 1-propenyl, isopropenyl, 1-butenyl, 2-butenyl, 2-methylallyl, and the like.

As used herein, the term “lower alkynyl” refers to an optionally branched alkynyl moiety having from 2 to 6 carbon atoms, such as ethynyl, 2-propynyl, and the like.

As used herein, the term “optionally fused” refers to a cyclic chemical group that may be fused with a ring system, such as cycloalkyl, heterocycloalkyl, aryl, or heteroaryl. Exemplary ring systems that may be fused to an optionally fused chemical group include, e.g., indolyl, isoindolyl, benzofuranyl, isobenzofuranyl, benzothiophenyl, benzoxazolyl, benzothiazolyl, benzoisoxazolyl, benzoisothiazolyl, indazolyl, benzimidazolyl, quinolinyl, isoquinolinyl, phthalazinyl, quinoxalinyl, quinazolinyl, cinnolinyl, indolizinyl, naphthyridinyl, pteridinyl, indanyl, naphtyl, 1,2,3,4-tetrahydronaphthyl, indolinyl, isoindolinyl, 2,3,4,5-tetrahydrobenzo[b]oxepinyl, 6,7,8,9-tetrahydro-5H-benzocycloheptenyl, chromanyl, and the like.

As used herein, the term “optionally substituted” refers to a chemical moiety that may have one or more (e.g., 1, 2, 3, 4, 5, 6, 7, 8, 9, 10, or more chemical substituents, such as lower alkyl, lower alkenyl, lower alkynyl, cycloalkyl, heterocyclolalkyl, aryl, alkylaryl, heteroaryl, alkylheteroaryl, amino, ammonium, acyl, acyloxy, acylamino, aminocarbonyl, alkoxycarbonyl, ureido, carbamate, sulfinyl, sulfonyl, alkoxy, sulfanyl, halogen, carboxy, trihalomethyl, cyano, hydroxy, mercapto, nitro, and the like. An optionally substituted chemical moiety may contain, e.g., neighboring substituents that have undergone ring closure, such as ring closure of vicinal functional substituents, thus forming, e.g., lactams, lactones, cyclic anhydrides, acetals, thioacetals, or aminals formed by ring closure, for instance, in order to generate protecting group.

As used herein, the term “sulfinyl” refers to the chemical moiety “—S(O)—R” in which R represents, e.g., hydrogen, aryl, heteroaryl, optionally substituted alkyl, optionally substituted alkenyl, or optionally substituted alkynyl.

As used herein, the term “sulfonyl” refers to the chemical moiety “—SO₂—R” in which R represents, e.g., hydrogen, aryl, heteroaryl, optionally substituted alkyl, optionally substituted alkenyl, or optionally substituted alkynyl.

One of skill in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure is to be understood as representing any such isomeric or isotopic form, individually or in combination.

### BRIEF DESCRIPTION OF THE FIGURES

FIG. 1 is a graph showing the effects of extended administration of a GnRH antagonist, 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid, on endometriosis-associated pain in human female human patients, as described in Example 1, below. Values shown represent the mean±95% confidence interval for pain scores from baseline through 52 weeks of continuous, once-daily treatment.

FIG. 2 is a graph showing the effects of extended administration of a GnRH antagonist, 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid, on bone mineral density (BMD) in human female human patients, as described in Example 1, below. Values represent the percent change in BMD as assessed in the lumbar spine.

FIGS. 3A and 3B are graphs showing the effects of extended administration of a GnRH antagonist, 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid, on quality of life in human female human patients, as described in Example 1, below. FIG. 3A shows the effects of GnRH antagonist treatment on quality of life after 12 weeks of continuous treatment, and FIG. 3B shows the effects of GnRH antagonist treatment on quality of life after 52 weeks of continuous treatment. Quality of life was assessed at both timepoints using the Endometriosis Health Profile-30 questionnaire.

FIG. 4 is a graph showing the response rate among endometriosis patients treated with 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid in accordance with the protocol described in Example 1, below. “Responders” were considered to be those patients that exhibited at least a 30% reduction in overall pelvic pain relative to baseline.

FIG. 5 is a graph showing the improvement in quality of life achieved among endometriosis patients treated with 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid in accordance with the protocol described in Example 1, below. Quality of life is measured in FIG. 5 using the Patient Global Impression of Change (PGIC) survey. Proportions of patients reporting “much” or “very much” improvement are shown along the y axis.

FIG. 6 is a graph showing the effects of continuous administration of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid on overall pelvic pain among endometriosis patients treated in accordance with the protocol described in Example 1, below.

FIG. 7 is a graph showing the effects of continuous administration of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid on dysmenorrhea among endometriosis patients treated in accordance with the protocol described in Example 1, below.

FIG. 8 is a graph showing the effects of continuous administration of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid on non-menstrual pelvic pain among endometriosis patients treated in accordance with the protocol described in Example 1, below.

FIG. 9 is a graph showing the effects of continuous administration of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid on dyspareunia among endometriosis patients treated in accordance with the protocol described in Example 1, below.

FIG. 10 is a graph showing the effects of continuous administration of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid on dyschezia among endometriosis patients treated in accordance with the protocol described in Example 1, below.

FIG. 11 is a graph showing the effects of continuous administration of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid on difficulty in doing daily activities among endometriosis patients treated in accordance with the protocol described in Example 1, below.

FIG. 12 is a graph showing the effects of continuous administration of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid on total cholesterol levels among endometriosis patients treated in accordance with the protocol described in Example 1, below. Values along the y axis represent the mean (±95% confidence interval) serum lipid level at each indicated timepoint.

FIG. 13 is a graph showing the effects of continuous administration of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid on serum triglyceride levels among endometriosis patients treated in accordance with the protocol described in Example 1, below. Values along the y axis represent the mean (±95% confidence interval) serum lipid level at each indicated timepoint.

FIG. 14 is a graph showing the effects of continuous administration of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid on high-density lipoprotein-cholesterol levels among endometriosis patients treated in accordance with the protocol described in Example 1, below. Values along the y axis represent the mean (±95% confidence interval) serum lipid level at each indicated timepoint.

FIG. 15 is a graph showing the effects of continuous administration of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid on low-density lipoprotein-cholesterol levels among endometriosis patients treated in accordance with the protocol described in Example 1, below. Values along the y axis represent the mean (±95% confidence interval) serum lipid level at each indicated timepoint.

FIG. 16 is a graph showing the effects of continuous administration of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid on the ratio of low-density lipoprotein-cholesterol level to high-density lipoprotein-cholesterol level among endometriosis patients treated in accordance with the protocol described in Example 1, below. Values along the y axis represent the mean (±95% confidence interval) lipid ratio at each indicated timepoint.

FIG. 17 is a graph showing the effects of continuous administration of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid on serum estradiol level among endometriosis patients treated in accordance with the protocol described in Example 1, below. Values along the y axis represent the mean (±95% confidence interval) lipid ratio at each indicated timepoint. FAS denotes the “full analysis set” of patients, which are those that were randomized and treated with the GnRH antagonist for the initial 24 weeks of the study described in Example 1. TEAS denotes the “treatment extension analysis set” of patients, which are those that completed 52 weeks of continuous treatment with the GnRH antagonist.

FIG. 18 is a graph showing the effect of compound (VI) on menstrual bleeding patterns in uterine fibroids patients treated with compound (VI) for 24 weeks in accordance with the procedure described in Example 3, below. Values along the y axis represent the proportion of patients exhibiting menstrual blood loss of less than ≤80 mL, as assessed by way of the alkaline hematin method, and a 50% reduction in menstrual blood loss relative to baseline. Error bars represent 95% confidence intervals.

FIG. 19 is a graph showing the effect of compound (VI) on menstrual bleeding patterns in uterine fibroids patients treated with compound (VI) for 52 weeks in accordance with the procedure described in Example 3, below. Values along the y axis represent the proportion of patients exhibiting menstrual blood loss of less than ≤80 mL, as assessed by way of the alkaline hematin method, and a 50% reduction in menstrual blood loss relative to baseline. Error bars represent 95% confidence intervals.

FIGS. 20A and 20B are graphs showing the effect of compound (VI) on menstrual bleeding patterns in uterine fibroids patients treated with compound (VI) for 52 weeks in accordance with the procedure described in Example 3, below. Particularly, FIGS. 20A and 20B show the effect of compound (VI) on menstrual blood loss as assessed at various timepoints during the PRIMROSE 1 and PRIMROSE 2 trials described in Example 3, below. Values along the y axis represent the average change from baseline (CFB) in menstrual blood loss. Error bars represent 95% confidence intervals.

FIGS. 21A and 21B are graphs showing the effect of compound (VI) on pain levels in uterine fibroids patients treated with compound (VI) for 24 weeks in accordance with the procedure described in Example 3, below. Particularly, FIGS. 21A and 21B show the effect of compound (VI) on pain as assessed at various timepoints during the PRIMROSE 1 and PRIMROSE 2 trials by way of a Verbal Rating Score (VRS). Values along the y axis represent the average change from baseline (CFB) in pain score (FIG. 21A) and the proportion of patients with a score of 1 or less after 24 weeks of treatment (FIG. 21B).

FIG. 22 is a graph showing the effect of compound (VI) on bone mineral density levels in uterine fibroids patients treated with compound (VI) for 24 weeks and 52 weeks in accordance with the procedure described in Example 3, below. Bone mineral density was assessed in the lumbar spine. Values along the y axis represent the average change from baseline (CFB) in bone mineral density throughout the PRIMROSE 1 and PRIMROSE 2 studies, descried in Example 3.

### DETAILED DESCRIPTION

The compositions and methods of the present disclosure can be used to treat a variety of estrogen-dependent disorders, including uterine fibroids and endometriosis, among others. For example, using the compositions and methods described herein, a patient, such as a female human patient, may be administered a gonadotropin-releasing hormone (GnRH) antagonist in order to not only treat the cause of the estrogen-dependent pathology, but also to alleviate one or more symptoms associated these conditions. Exemplary disease that are induced by elevated estrogen production, and that can be treated using the compositions and methods described herein, include, without limitation, uterine fibroids, endometriosis (e.g., rectovaginal endometriosis), and adenomyosis, among others.

GnRH antagonists that may be used for the treatment of an estrogen-dependent disorder as described herein include optionally substituted thieno[3,4d]pyrimidine derivatives, such as 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid or the choline salt thereof. Other GnRH antagonists useful in conjunction with the compositions and methods of the disclosure include optionally substituted 3-aminoalkyl pyrimidine-2,4(1H,3H)-dione derivatives, such as sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate, also referred to as elagolix, or the carboxylic acid conjugate thereof. In some embodiments, the GnRH antagonist is an optionally substituted thieno[2,3d]pyrimidine derivative, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, also referred to as relugolix, or a pharmaceutically acceptable salt thereof. In some embodiments, the GnRH antagonist is an optionally substituted propane-1,3-dione derivative, such as (2R)—N-{5-[3-(2,5-difluorophenyl)-2-(1,3-dihydro-2H-benzimidazol-2-ylidene)-3-oxopropanoyl]-2-fluorobenzene-1-sulfonyl}-2-hydroxypropanimidamide, also referred to as opigolix or ASP-1707. Additional GnRH antagonists that may be used in conjunction with the compositions and methods described herein include SKI2670 and BAY-784, as well as derivatives and variants thereof, among others.

Estrogen-dependent diseases, such as uterine fibroids, endometriosis (e.g., rectovaginal endometriosis), and adenomyosis, among others described herein, may be effectuated by circulating concentrations of β17-estradiol (E2) in excess of about 60 μg/ml. Using the compositions and methods described herein, a GnRH antagonist may be administered to the patient so as to suppress E2 production in order to correct the underlying physiological cause of the disease and ameliorate one or more symptoms associated therewith. However, excessive depletion of endogenous E2 (for example, to levels of less than about 10 μg/ml) may lead to undesirable side effects, such as a reduction in bone mineral density or an elevation in serum cholesterol concentration.

The present disclosure is based, in part, on the surprising observation that administration of a GnRH antagonist, such as an optionally substituted thieno[3,4d]pyrimidine derivative (e.g., 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid or the choline salt thereof), can be periodically administered to a patient (e.g., a human patient suffering from an estrogen-dependent disease) over extended period of time, such as over the course of a treatment period lasting multiple years. Unexpectedly, administration of the GnRH antagonist over such lengthy treatment periods effectuates a sustained reduction in estrogen-dependent disease symptomology without inducing side effects that are associated with estrogen depletion, such as loss of bone mineral density or increases in serum cholesterol.

The sections that follow provide a detailed description of GnRH antagonists and other agents that may be used in conjunction with the compositions and methods of the disclosure, as well as a description of various estrogen-dependent diseases that may be treated using these therapeutics.

### GnRH Antagonists

### Thieno[3,4d]pyrimidines

GnRH antagonists for use with the compositions and methods described herein include thieno[3,4d]pyrimidine derivatives and variants, such as those described in U.S. Pat. No. 9,040,693, the disclosure of which is incorporated herein by reference in its entirety. Exemplary GnRH antagonists include those represented by formula (I)

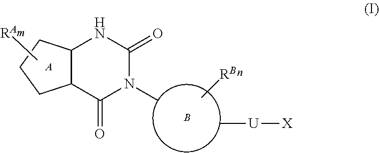

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group;

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof.

In some embodiments, the ring A is a thiophene ring represented by formula (IIa)

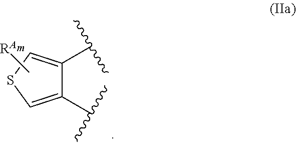

In some embodiments, m is 1 or 2. In some embodiments, m is 1. For instance, the ring A may be an optionally substituted thiophene ring represented by formula (IIb)

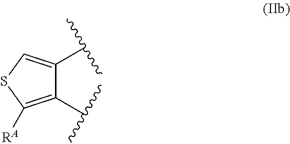

Each R^{A} may independently be, for example, a halogen atom (e.g., fluorine, chlorine, bromine, or iodine), an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group. In some embodiments, each R^{A} is COOH or pharmaceutically acceptable salt thereof.

In some embodiments, ring B is an optionally substituted benzene ring, pyridine ring, or thiophene ring. For instance, ring B may be represented by a formula selected from the group consisting of:

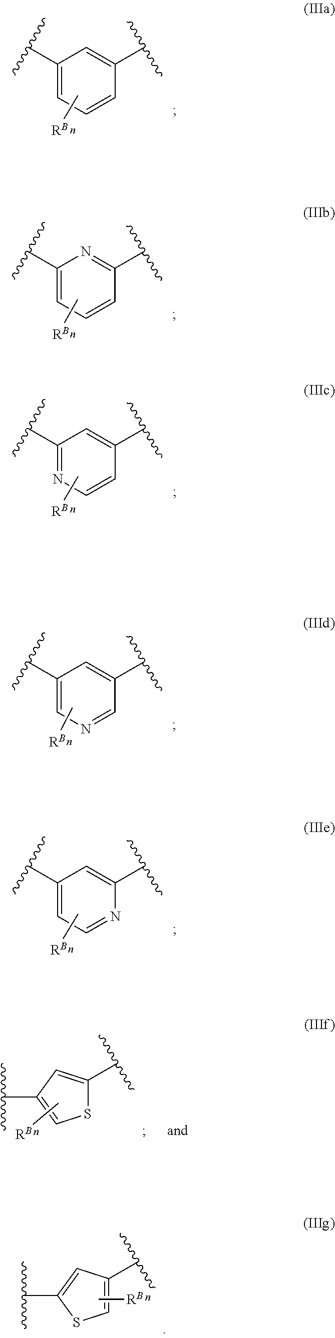

In some embodiments, n is 1 or 2. For instance, in some embodiments, n is 1. Ring B may be, for example, represented by a formula selected from the group consisting of:

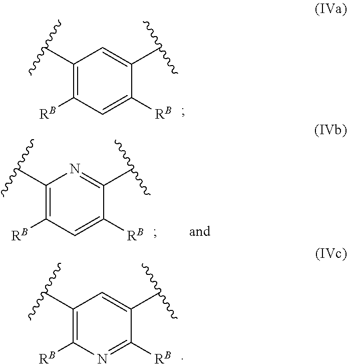

In some embodiments, each R^{B} is independently a halogen atom, an optionally substituted lower alkyl group, or OW⁴, wherein each W⁴ is independently a hydrogen atom or an optionally substituted lower alkyl group. For instance, each R^{B} may be independently a fluorine atom, chlorine atom, bromine atom, methyl group, or methoxy group.

In some embodiments, U is a single bond. X may be, for example, a group represented by —O-L-Y. L may be, for example, a methylene group. In some embodiments, Y is an optionally substituted benzene ring represented by formula (V)

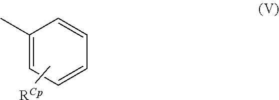

wherein each R^{C} is independently a halogen atom, an optionally substituted lower alkyl group, or OW⁹, wherein each W⁹ is independently a hydrogen atom or an optionally substituted lower alkyl group; and

p is an integer from 0 to 3.

In some embodiments, Y is a substituted benzene ring represented by formula (Va)

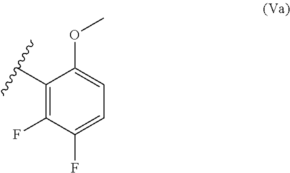 [SMILES: *c1c(OC)ccc(F)c1F]

For example, GnRH antagonists that may be used for the treatment of the endometrial growth disorders described herein include those thieno[3,4d]pyrimidine compounds described in Table 1, below. The synthesis and characterization of these compounds is reported, for instance, in U.S. Pat. No. 9,040,693, incorporated herein by reference.

TABLE 1
Exemplary Thieno[3,4d]pyrimidine GnRH Antagonists Useful for the Treatment of Estrogen-Dependent Diseases

| No. | Compound | Reported ¹H NMR spectral properties |
|---|---|---|
| 1 | 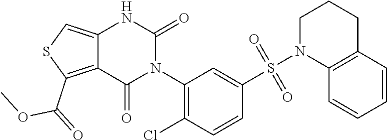 [SMILES: COC(=O)c1scc2[nH]c(=O)n(-c3cc(S(=O)(=O)N4CCCc5ccccc54)ccc3Cl)c(=O)c12] | (CDCl₃) 1.6-1.75 (2H, m), 2.45-2.6 (2H, m), 3.7-3.85 (2H, m), 3.94 (3H, s), 6.82 (1H, s), 7.0-7.15 (2H, m), 7.15-7.25 (1H, m), 7.49 (1H, dd, J = 8.4 Hz, 2.0 Hz), 7.54 (1H, d, J = 8.4 Hz), 7.6 (1H, d, J = 2.0 Hz), 7.7-7.8 (1H, m), 9.47 (1H, s) |
| 2 | 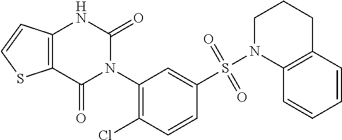 [SMILES: O=c1[nH]c2ccsc2c(=O)n1-c1cc(S(=O)(=O)N2CCCc3ccccc32)ccc1Cl] | (CDCl₃) 1.6-1.75 (2H, m), 2.4-2.6 (2H, m), 3.7-3.8 (1H, m), 3.8-3.9 (1H, m), 6.82 (1H, d, J = 5.2 Hz), 7.0-7.15 (2H, m), 7.15-7.25 (1H, m), 7.46 (1H, dd, J = 8.3 Hz, 2.1 Hz), 7.54 (1H, d, J = 8.3 Hz), 7.65 (1H, d, J = 2.1 Hz), 7.7-7.8 (2H, m), 9.65 (1H, s) |
| 3 | 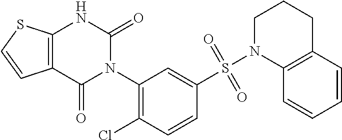 [SMILES: O=c1[nH]c2sccc2c(=O)n1-c1cc(S(=O)(=O)N2CCCc3ccccc32)ccc1Cl] | (CDCl₃) 1.6-1.75 (2H, m), 2.4-2.6 (2H, m), 3.7-3.9 (2H, m), 6.89 (1H, d, J = 5.8 Hz), 7.0-7.15 (2H, m), 7.15-7.25 (1H, m), 7.32 (1H, d, J = 5.8 Hz), 7.46 (1H, dd, J = 8.4 Hz, 2.1 Hz), 7.55 (1H, d, J = 8.4 Hz), 7.64 (1H, d, J = 2.1 Hz), 7.75- 7.8 (1H, m), 9.54 (1H, brs) |
| 4 | 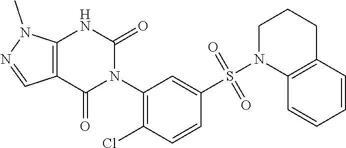 [SMILES: Cn1ncc2c(=O)n(-c3cc(S(=O)(=O)N4CCCc5ccccc54)ccc3Cl)c(=O)[nH]c21] | (CDCl₃) 1.6-1.8 (2H, m), 2.4-2.6 (2H, m), 3.65-3.8 (4H, m), 3.8-3.9 (1H, m), 7.0-7.15 (2H, m), 7.15-7.25 (1H, m), 7.49 (1H, dd, J = 8.4 Hz, 1.9 Hz), 7.55 (1H, d, J = 8.4 Hz), 7.59 (1H, d, J = 1.9 Hz), 7.75 (1H, d, J = 7.5 Hz), 7.91 (1H, s) |
| 5 | 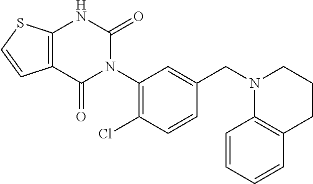 [SMILES: O=c1[nH]c2sccc2c(=O)n1-c1cc(CN2CCCc3ccccc32)ccc1Cl] | (CDCl₃) 1.95-2.05 (2H, m), 2.75-2.85 (2H, m), 3.3-3.35 (2H, m), 4.48 (2H, s), 6.5 (1H, d, J = 8.4 Hz), 6.55-6.56 (1H, m), 6.81 (1H, d, J = 5.6 Hz), 6.9-7.0 (2H, m), 7.2-7.35 (3H, m), 7.51 (1H, d, J = 8.4 Hz), 9.94 (1H, brs) |
| 6 | 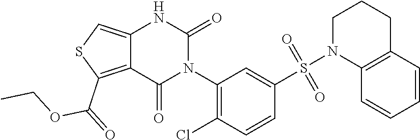 [SMILES: CCOC(=O)c1scc2[nH]c(=O)n(-c3cc(S(=O)(=O)N4CCCc5ccccc54)ccc3Cl)c(=O)c12] | (CDCl₃) 1.38 (3H, t, J = 7.1 Hz), 1.6- 1.75 (2H, m), 2.4-2.6 (2H, m), 3.7-3.85 (2H, m), 4.41 (2H, q, J = 7.1 Hz), 6.82 (1H, s), 7.0-7.15 (2H, m), 7.15-7.25 (1H, m), 7.47 (1H, dd, J = 8.5 Hz, 2.2 Hz), 7.53 (1H, d, J = 8.5 Hz), 7.6 (1H, d, J = 2.2 Hz), 7.7-7.8 (1H, m), 9.11 (1H, s) |
| 7 | 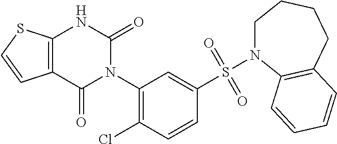 [SMILES: O=c1[nH]c2sccc2c(=O)n1-c1cc(S(=O)(=O)N2CCCCc3ccccc32)ccc1Cl] | (CDCl₃) 1.2-1.35 (2H, m), 1.75-1.9 (2H, m), 2.4-2.55 (2H, m), 3.2-4.3 (2H, br), 6.87 (1H, d, J = 5.3 Hz), 7.1-7.2 (3H, m), 7.25-7.35 (2H, m), 7.63 (1H, d, J = 8.5 Hz), 7.69 (1H, dd, J = 8.5 Hz, 1.9 Hz), 7.78 (1H, d, J = 1.9 Hz), 9.5-10.2 (1H, br) |
| 8 | 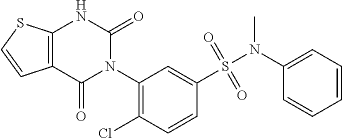 [SMILES: CN(c1ccccc1)S(=O)(=O)c1ccc(Cl)c(-n2c(=O)[nH]c3sccc3c2=O)c1] | (CDCl₃) 3.2 (3H, s), 6.87 (1H, d, J = 5.4 Hz), 7.1-7.15 (2H, m), 7.2-7.35 (4H, m), 7.54 (1H, dd, J = 8.4 Hz, 2.0 Hz), 7.6 (1H, d, J = 2.0 Hz), 7.64 (1H, d, J = 8.4 Hz), 9.5-11.0 (1H, br) |
| 9 | 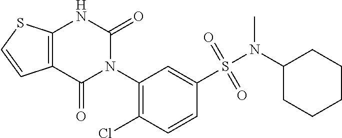 [SMILES: CN(C1CCCCC1)S(=O)(=O)c1ccc(Cl)c(-n2c(=O)[nH]c3sccc3c2=O)c1] | (CDCl₃) 0.9-1.8 (10H, m), 2.77 (3H, s), 3.7-3.8 (1H, m), 6.87 (1H, d, J = 5.7 Hz), 7.3 (1H, d, J = 5.7 Hz), 7.69 (1H, d, J = 8.1 Hz), 7.8-7.9 (2H, m), 9.5-10.5 (1H, br) |
| 10 | 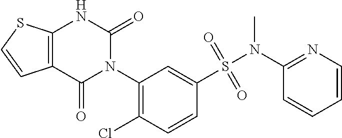 [SMILES: CN(c1ccccn1)S(=O)(=O)c1ccc(Cl)c(-n2c(=O)[nH]c3sccc3c2=O)c1] | (DMSO-d₆) 3.26 (3H, s), 7.19 (1H, d, J = 5.7 Hz), 7.22 (1H, d, J = 5.7 Hz), 7.25- 7.3 (1H, m), 7.45-7.55 (1H, m), 7.62 (1H, dd, J = 8.5 Hz, 2.1 Hz), 7.8-7.9 (2H, m), 8.04 (1H, d, J = 2.1 Hz), 8.3-8.4 (1H, m), 12.54 (1H, s) |
| 11 | 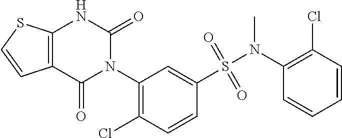 [SMILES: CN(c1ccccc1Cl)S(=O)(=O)c1ccc(Cl)c(-n2c(=O)[nH]c3sccc3c2=O)c1] | (DMSO-d₆) 3.16 (3H, s), 7.05 (1H, d, J = 7.8 Hz), 7.15-7.45 (4H, m), 7.5-7.8 (1H, m), 7.75-7.81 (3H, m), 12.55 (1H, s) |
| 12 | 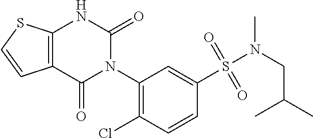 [SMILES: CC(C)CN(C)S(=O)(=O)c1ccc(Cl)c(-n2c(=O)[nH]c3sccc3c2=O)c1] | (DMSO-d₆) 0.8-0.9 (6H, m), 1.75-1.95 (1H, m), 2.6-2.8 (5H, m), 7.15-7.25 (2H, m), 7.8-7.95 (2H, m), 8.0-8.1 (1H, m), 12.53 (1H, s) |
| 13 | 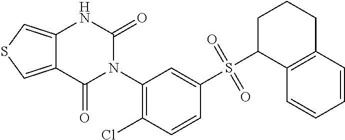 [SMILES: O=c1[nH]c2cscc2c(=O)n1-c1cc(S(=O)(=O)C2CCCc3ccccc32)ccc1Cl] | (DMSO-d₆) 1.6-1.75 (2H, m), 2.4-2.55 (2H, m), 3.7-3.85 (2H, m), 6.95 (1H, d, J = 3.1 Hz), 7.05-7.25 (3H, m), 7.52 (1H, dd, J = 8.5 Hz, 2.0 Hz), 7.56 (1H, d, J = 8.1 Hz), 7.78 (1H, d, J = 8.5 Hz), 8.04 (1H, d, J = 2.0 Hz), 8.52 (1H, d, J = 3.1 Hz), 11.53 (1H, s) |
| 14 | 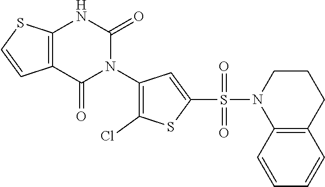 [SMILES: O=c1[nH]c2sccc2c(=O)n1-c1cc(S(=O)(=O)N2CCCc3ccccc32)sc1Cl] | (CDCl₃) 1.75-1.9 (2H, m), 2.55-2.7 (2H, m), 3.75-3.95 (2H, m), 6.9 (1H, d, J = 5.5 Hz), 7.05-7.25 (4H, m), 7.29 (1H, d, J = 5.5 Hz), 7.74 (1H, d, J = 8.2 Hz), 10.17 (1H, s) |
| 15 | 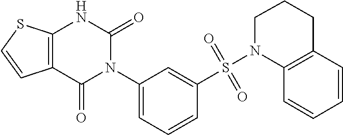 [SMILES: O=c1[nH]c2sccc2c(=O)n1-c1cccc(S(=O)(=O)N2CCCc3ccccc32)c1] | (DMSO-d₆) 1.6-1.7 (2H, m), 2.4-2.5 (2H, m), 3.7-3.8 (2H, m), 7.05-7.1 (2H, m), 7.1-7.25 (3H, m), 7.45-7.65 (4H, m), 7.74 (1H, d, J = 1.0 Hz), 12.37 (1H, s) |
| 16 | 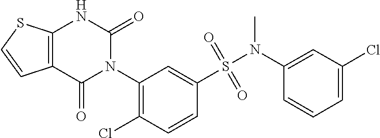 [SMILES: CN(c1cccc(Cl)c1)S(=O)(=O)c1ccc(Cl)c(-n2c(=O)[nH]c3sccc3c2=O)c1] | (DMSO-d₆) 3.18 (3H, s), 7.0-7.1 (1H, m), 7.15-7.25 (3H, m), 7.35-7.4 (2H, m), 7.6 (1H, dd, J = 8.5 Hz, 2.3 Hz), 7.87 (1H, d, J = 8.5 Hz), 7.96 (1H, d, J = 2.3 Hz), 12.54 (1H, s) |
| 17 | 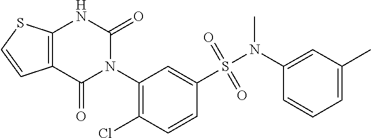 [SMILES: Cc1cccc(N(C)S(=O)(=O)c2ccc(Cl)c(-n3c(=O)[nH]c4sccc4c3=O)c2)c1] | (DMSO-d₆) 2.26 (3H, s), 3.15 (3H, s), 6.8-6.95 (2H, m), 7.05-7.25 (4H, m), 7.6 (1H, dd, J = 8.4 Hz, 2.3 Hz), 7.8-7.9 (2H, m), 12.54 (1H, s) |
| 18 | 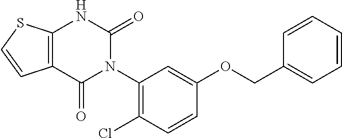 [SMILES: O=c1[nH]c2sccc2c(=O)n1-c1cc(OCc2ccccc2)ccc1Cl] | (DMSO-d₆) 5.1 (2H, s), 7.14 (1H, dd, J = 9.0 Hz, 2.9 Hz), 7.18 (1H, d, J = 5.6 Hz), 7.22 (1H, d, J = 5.6 Hz), 7.25 (1H, d, J = 2.9 Hz), 7.3-7.5 (5H, m), 7.53 (1H, d, J = 9.0 Hz), 12.48 (1H, s) |
| 19 | 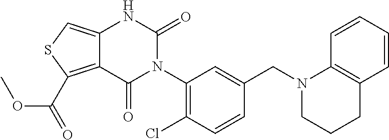 [SMILES: COC(=O)c1scc2[nH]c(=O)n(-c3cc(CN4CCCc5ccccc54)ccc3Cl)c(=O)c12] | (DMSO-d₆) 1.85-2.0 (2H, m), 2.7-2.8 (2H, m), 3.3-3.4 (2H, m), 3.81 (3H, s), 4.5 (2H, s), 6.4-6.5 (2H, m), 6.85-6.95 (2H, m), 7.2 (1H, s), 7.31 (1H, dd, J = 8.2 Hz, 2.1 Hz), 7.4 (1H, d, J = 2.1 Hz), 7.56 (1H, d, J = 8.2 Hz), 11.61 (1H, s) |
| 20 | 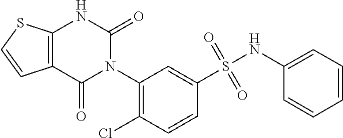 [SMILES: O=c1[nH]c2sccc2c(=O)n1-c1cc(S(=O)(=O)Nc2ccccc2)ccc1Cl] | (DMSO-d₆) 7.05-7.3 (7H, m), 7.75-7.9 (2H, m), 7.95-8.05 (1H, m), 10.48 (1H, s), 12.56 (1H, s) |
| 21 | 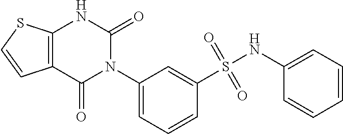 [SMILES: O=c1[nH]c2sccc2c(=O)n1-c1cccc(S(=O)(=O)Nc2ccccc2)c1] | (DMSO-d₆) 7.0-7.3 (7H, m), 7.55-7.7 (2H, m), 7.75-7.85 (2H, m), 10.4 (1H, s), 12.4 (1H, s) |
| 22 | 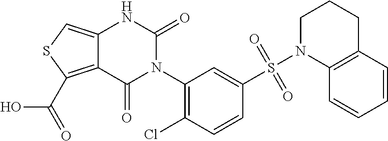 [SMILES: O=C(O)c1scc2[nH]c(=O)n(-c3cc(S(=O)(=O)N4CCCc5ccccc54)ccc3Cl)c(=O)c12] | (DMSO-d₆) 1.6-1.7 (2H, m), 2.4-2.55 (2H, m), 3.7-3.8 (2H, m), 7.05-7.25 (3H, m), 7.39 (1H, s), 7.55 (1H, d, J = 8.2 Hz), 7.6 (1H, dd, J = 8.5 Hz, 2.4 Hz), 7.83 (1H, d, J = 8.5 Hz), 8.09 (1H, d, J = 2.4 Hz), 12.03 (1H, s), 14.23 (1H, s) |
| 23 | 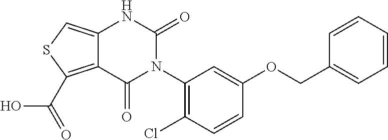 [SMILES: O=C(O)c1scc2[nH]c(=O)n(-c3cc(OCc4ccccc4)ccc3Cl)c(=O)c12] | (DMSO-d₆) 5.11 (2H, s), 7.19 (1H, dd, J = 9.0 Hz, 2.9 Hz), 7.3-7.45 (5H, m), 7.45-7.5 (2H, m), 7.57 (1H, d, J = 9.0 Hz), 12.04 (1H, s), 14.45 (1H, brs) |
| 24 | 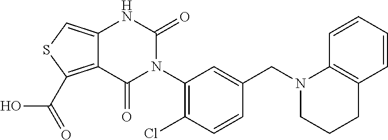 [SMILES: O=C(O)c1scc2[nH]c(=O)n(-c3cc(CN4CCCc5ccccc54)ccc3Cl)c(=O)c12] | (DMSO-d₆) 1.85-2.0 (2H, m), 2.7-2.8 (2H, m), 3.3-3.4 (2H, m), 4.52 (2H, s), 6.44 (1H, d, J = 8.2 Hz), 6.45-6.5 (1H, m), 6.85-6.95 (2H, m), 7.36 (1H, d, J = 8.2 Hz), 7.38 (1H, s), 7.48 (1H, d, J = 2.1 Hz), 7.61 (1H, d, J = 8.2 Hz), 12.0 (1H, s), 14.45 (1H, brs) |
| 25 | 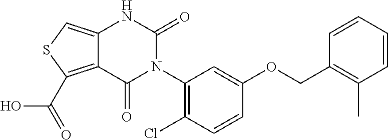 [SMILES: Cc1ccccc1COc1ccc(Cl)c(-n2c(=O)[nH]c3csc(C(=O)O)c3c2=O)c1] | (DMSO-d₆) 2.33 (3H, s), 5.09 (2H, s), 7.15-7.3 (4H, m), 7.35 (1H, d, J = 2.7 Hz), 7.41 (1H, s), 7.43 (1H, d, J = 7.7 Hz), 7.58 (1H, d, J = 9.0 Hz), 12.04 (1H, s), 14.44 (1H, s) |
| 26 | 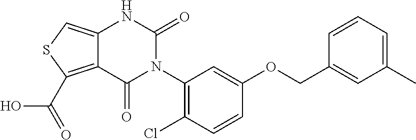 [SMILES: Cc1cccc(COc2ccc(Cl)c(-n3c(=O)[nH]c4csc(C(=O)O)c4c3=O)c2)c1] | (DMSO-d₆) 2.32 (3H, s), 5.07 (2H, s), 7.1-7.35 (6H, m), 7.41 (1H, s), 7.57 (1H, d, J = 8.7 Hz), 12.04 (1H, s), 14.45 (1H, brs) |
| 27 | 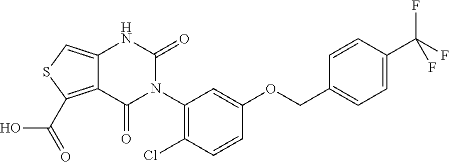 [SMILES: O=C(O)c1scc2[nH]c(=O)n(-c3cc(OCc4ccc(C(F)(F)F)cc4)ccc3Cl)c(=O)c12] | (DMSO-d₆) 5.24 (2H, s), 7.2 (1H, dd, J = 8.8 Hz, 3.0 Hz), 7.35 (1H, d, J = 3.0 Hz), 7.41 (1H, s), 7.59 (1H, d, J = 8.8 Hz), 7.69 (2H, d, J = 8.2 Hz), 7.78 (2H, d, J = 8.2 Hz), 12.04 (1H, s), 14.43 (1H, s) |
| 28 | 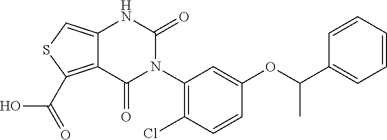 [SMILES: CC(Oc1ccc(Cl)c(-n2c(=O)[nH]c3csc(C(=O)O)c3c2=O)c1)c1ccccc1] | (DMSO-d₆) 1.5-1.6 (3H, m), 5.45-5.55 (1H, m), 7.0-7.1 (1H, m), 7.23 (1H, dd, J = 6.1 Hz, 2.9 Hz), 7.25-7.5 (7H, m), 11.95-12.1 (1H, m), 14.42 (1H, s) |
| 29 | 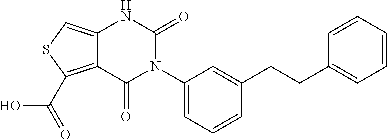 [SMILES: O=C(O)c1scc2[nH]c(=O)n(-c3cccc(CCc4ccccc4)c3)c(=O)c12] | (DMSO-d₆) 2.85-3.0 (4H, m), 7.15-7.35 (8H, m), 7.38 (1H, s), 7.41 (1H, t, J = 7.7 Hz), 11.92 (1H, s), 14.94 (1H, brs) |
| 30 | 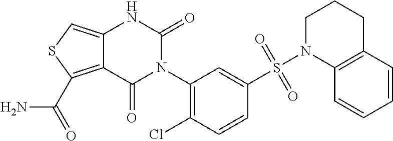 [SMILES: NC(=O)c1scc2[nH]c(=O)n(-c3cc(S(=O)(=O)N4CCCc5ccccc54)ccc3Cl)c(=O)c12] | (DMSO-d₆) 1.6-1.7 (2H, m), 2.4-2.55 (2H, m), 3.7-3.85 (2H, m), 7.0-7.2 (3H, m), 7.24 (1H, s), 7.56 (1H, d, J = 8.3 Hz), 7.59 (1H, dd, J = 8.5, 2.1 Hz), 7.81 (1H, d, J = 8.5 Hz), 8.05-8.15 (2H, m), 9.55 (1H, s), 11.81 (1H, s) |
| 31 | 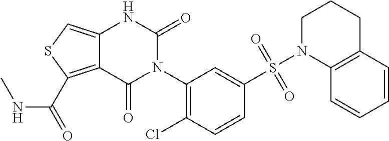 [SMILES: CNC(=O)c1scc2[nH]c(=O)n(-c3cc(S(=O)(=O)N4CCCc5ccccc54)ccc3Cl)c(=O)c12] | (CDCl₃) 1.65-1.75 (2H, m), 2.4-2.6 (2H, m), 2.99 (3H, d, J = 4.7 Hz), 3.7-3.9 (2H, m), 6.91 (1H, s), 6.95-7.15 (2H, m), 7.15-7.25 (1H, m), 7.5-7.65 (3H, m), 7.7-7.8 (1H, m), 8.91 (1H, s), 10.05- 10.15 (1H, m) |
| 32 | 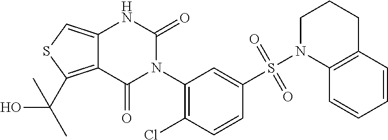 [SMILES: CC(C)(O)c1scc2[nH]c(=O)n(-c3cc(S(=O)(=O)N4CCCc5ccccc54)ccc3Cl)c(=O)c12] | (CDCl₃) 1.55 (6H, s), 1.6-1.75 (2H, m), 2.4-2.55 (2H, m), 3.7-3.9 (2H, m), 6.04 (1H, s), 6.49 (1H, s), 7.0-7.15 (2H, m), 7.15-7.25 (2H, m), 7.5-7.55 (1H, m), 7.55-7.6 (2H, m), 7.76 (1H, d, J = 8.3 Hz), 8.41 (1H, s) |
| 33 | 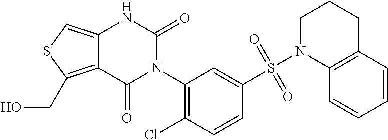 [SMILES: O=c1[nH]c2csc(CO)c2c(=O)n1-c1cc(S(=O)(=O)N2CCCc3ccccc32)ccc1Cl] | (DMSO-d₆) 1.6-1.75 (2H, m), 2.45-2.55 (2H, m), 3.7-3.85 (2H, m), 4.95-5.05 (2H, m), 5.99 (1H, t, J = 5.5 Hz), 6.73 (1H, s), 7.05-7.15 (2H, m), 7.15-7.25 (1H, m), 7.5-7.6 (2H, m), 7.78 (1H, d, J = 8.5 Hz), 7.97 (1H, d, J = 2.3 Hz), 11.41 (1H, s) |
| 34 | 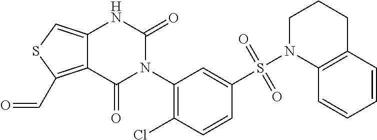 [SMILES: O=Cc1scc2[nH]c(=O)n(-c3cc(S(=O)(=O)N4CCCc5ccccc54)ccc3Cl)c(=O)c12] | (DMSO-d₆) 1.6-1.75 (2H, m), 2.45-2.55 (2H, m), 3.7-3.8 (2H, m), 7.05-7.15 (2H, m), 7.15-7.25 (1H, m), 7.5-7.6 (3H, m), 7.82 (1H, d, J = 8.5 Hz), 8.09 (1H, d, J = 2.1 Hz), 10.5-10.55 (1H, m), 11.88 (1H, s) |
| 35 | 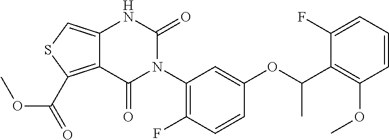 [SMILES: COC(=O)c1scc2[nH]c(=O)n(-c3cc(OC(C)c4c(F)cccc4OC)ccc3F)c(=O)c12] | (DMSO-d₆) 1.64 (3H, d, J = 6.5 Hz), 3.8- 3.9 (6H, m), 5.79 (1H, q, J = 6.5 Hz), 6.7-6.8 (1H, m), 6.85-6.95 (2H, m), 6.95- 7.05 (1H, m), 7.15-7.25 (2H, m), 7.25- 7.35 (1H, m), 11.63 (1H, s) |
| 36 | 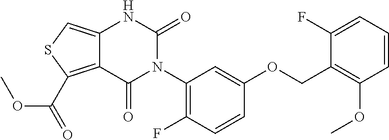 [SMILES: COC(=O)c1scc2[nH]c(=O)n(-c3cc(OCc4c(F)cccc4OC)ccc3F)c(=O)c12] | (DMSO-d₆) 3.83 (3H, s), 3.85 (3H, s), 4.99 (2H, s), 6.85-7.0 (2H, m), 7.1-7.2 (2H, m), 7.21 (1H, s), 7.25-7.35 (1H, m), 7.4-7.5 (1H, m), 11.68 (1H, s) |
| 37 | 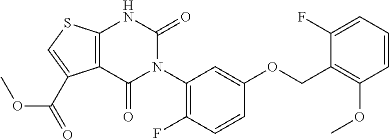 [SMILES: COC(=O)c1csc2[nH]c(=O)n(-c3cc(OCc4c(F)cccc4OC)ccc3F)c(=O)c12] | (DMSO-d₆) 3.75 (3H, s), 3.85 (3H, s), 4.99 (2H, s), 6.85-7.0 (2H, m), 7.1-7.2 (2H, m), 7.25-7.35 (1H, m), 7.4-7.5 (1H, m), 7.65 (1H, s), 12.63 (1H, s) |
| 38 | 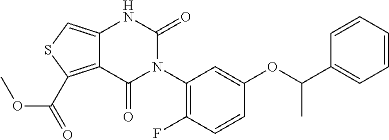 [SMILES: COC(=O)c1scc2[nH]c(=O)n(-c3cc(OC(C)c4ccccc4)ccc3F)c(=O)c12] | (DMSO-d₆) 1.54 (3H, d, J = 6.3 Hz), 3.8- 3.85 (3H, m), 5.46 (1H, q, J = 6.3 Hz), 6.9-7.0 (1H, m), 7.05-7.11 (1H, m), 7.15- 7.45 (7H, m), 11.63 (1H, s) |
| 39 | 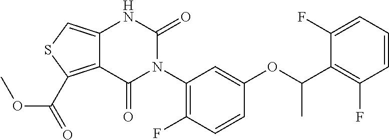 [SMILES: COC(=O)c1scc2[nH]c(=O)n(-c3cc(OC(C)c4c(F)cccc4F)ccc3F)c(=O)c12] | (DMSO-d₆) 1.7 (3H, d, J = 6.6 Hz), 3.8- 3.85 (3H, m), 5.7-5.8 (1H, m), 6.9-7.0 (1H, m), 7.05-7.15 (3H, m), 7.15-7.3 (2H, m), 7.35-7.5 (1H, m), 11.63 (1H, s) |
| 40 | 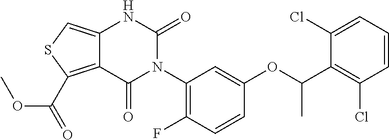 [SMILES: COC(=O)c1scc2[nH]c(=O)n(-c3cc(OC(C)c4c(Cl)cccc4Cl)ccc3F)c(=O)c12] | (DMSO-d₆) 1.71 (3H, d, J = 6.6 Hz), 3.82 (3H, s), 5.95-6.05 (1H, m), 6.8-6.9 (1H, m), 7.0-7.05 (1H, m), 7.15-7.3 (2H, m), 7.3-7.4 (1H, m), 7.45-7.5 (2H, m), 11.63 (1H, s) |
| 41 | 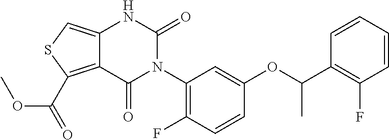 [SMILES: COC(=O)c1scc2[nH]c(=O)n(-c3cc(OC(C)c4ccccc4F)ccc3F)c(=O)c12] | (DMSO-d₆) 1.58 (3H, d, J = 6.3 Hz), 3.8- 3.85 (3H, m), 5.66 (1H, q, J = 6.3 Hz), 6.9-7.0 (1H, m), 7.05-7.15 (1H, m), 7.15-7.3 (4H, m), 7.3-7.4 (1H, m), 7.45- 7.55 (1H, m), 11.63 (1H, s) |
| 42 | 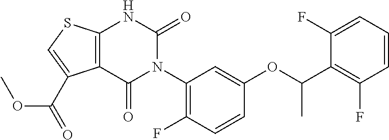 [SMILES: COC(=O)c1csc2[nH]c(=O)n(-c3cc(OC(C)c4c(F)cccc4F)ccc3F)c(=O)c12] | (DMSO-d₆) 1.69 (3H, d, J = 6.4 Hz), 3.7- 3.75 (3H, m), 5.7-5.8 (1H, m), 6.9-7.0 (1H, m), 7.05-7.15 (3H, m), 7.2-7.3 (1H, m), 7.35-7.5 (1H, m), 7.6-7.65 (1H, m), 12.59 (1H, s) |
| 43 | 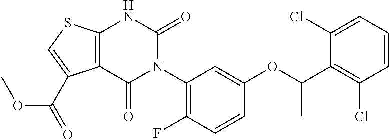 [SMILES: COC(=O)c1csc2[nH]c(=O)n(-c3cc(OC(C)c4c(Cl)cccc4Cl)ccc3F)c(=O)c12] | (DMSO-d₆) 1.71 (3H, d, J = 6.6 Hz), 3.74 (3H, s), 5.95-6.05 (1H, m), 6.8-6.9 (1H, m), 6.95-7.05 (1H, m), 7.2-7.3 (1H, m), 7.3-7.4 (1H, m), 7.4-7.5 (2H, m), 7.6-7.65 (1H, m), 12.59 (1H, s) |
| 44 | 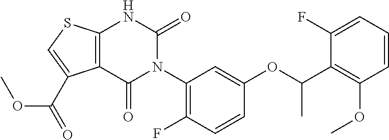 [SMILES: COC(=O)c1csc2[nH]c(=O)n(-c3cc(OC(C)c4c(F)cccc4OC)ccc3F)c(=O)c12] | (DMSO-d₆) 1.64 (3H, d, J = 6.7 Hz), 3.7- 3.8 (3H, m), 3.8-3.9 (3H, m), 5.75-5.85 (1H, m), 6.7-6.8 (1H, m), 6.85-6.95 (2H, m), 6.95-7.05 (1H, m), 7.15-7.25 (1H, m), 7.25-7.35 (1H, m), 7.63 (1H, s), 12.58 (1H, s) |
| 45 | 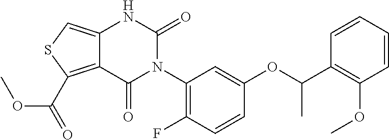 [SMILES: COC(=O)c1scc2[nH]c(=O)n(-c3cc(OC(C)c4ccccc4OC)ccc3F)c(=O)c12] | (DMSO-d₆) 1.51 (3H, d, J = 6.3 Hz), 3.8- 3.9 (6H, m), 5.6-5.7 (1H, m), 6.8-6.9 (1H, m), 6.9-7.0 (1H, m), 7.0-7.1 (2H, m), 7.15-7.3 (3H, m), 7.3-7.4 (1H, m), 11.6 (1H, s) |
| 46 | 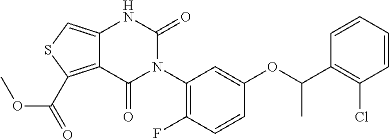 [SMILES: COC(=O)c1scc2[nH]c(=O)n(-c3cc(OC(C)c4ccccc4Cl)ccc3F)c(=O)c12] | (DMSO-d₆) 1.57 (3H, d, J = 6.3 Hz), 3.8- 3.85 (3H, m), 5.6-5.7 (1H, m), 6.8-6.9 (1H, m), 7.05-7.1 (1H, m), 7.15-7.3 (2H, m), 7.3-7.4 m), 7.45-7.5 (1H, m), 7.5-7.6 (1H, m), 11.61 (1H, s) |
| 47 | 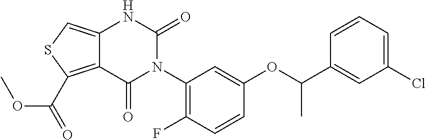 [SMILES: COC(=O)c1scc2[nH]c(=O)n(-c3cc(OC(C)c4cccc(Cl)c4)ccc3F)c(=O)c12] | (DMSO-d₆) 1.54 (3H, d, J = 6.2 Hz), 3.75-3.85 (3H, m), 5.45-5.55 (1H, m), 6.95-7.05 (1H, m), 7.05-7.15 (1H, m), 7.15-7.25 (2H, m), 7.3-7.45 (3H, m), 7.49 (1H, s), 11.61 (1H, s) |
| 48 | 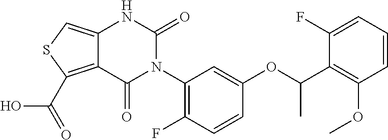 [SMILES: COc1cccc(F)c1C(C)Oc1ccc(F)c(-n2c(=O)[nH]c3csc(C(=O)O)c3c2=O)c1] | (DMSO-d₆) 1.65 (3H, d, J = 6.5 Hz), 3.8- 3.9 3H, m), 5.75-5.85 (1H, m), 6.7-6.8 (1H, m), 6.85-7.0 (2H, m), 7.05-7.1 (1H, m), 7.2-7.35 (2H, m), 7.37 (1H, d, J = 3.6 Hz), 12.01 (1H, s), 14.43 (1H, s) |
| 49 | 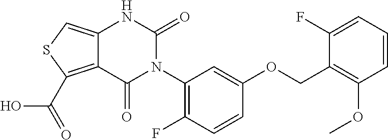 [SMILES: COc1cccc(F)c1COc1ccc(F)c(-n2c(=O)[nH]c3csc(C(=O)O)c3c2=O)c1] | (DMSO-d₆) 3.85 (3H, s), 5.0 (2H, s), 6.88 (1H, t, J = 8.7 Hz), 6.95 (1H, d, J = 8.7 Hz), 7.15-7.25 (2H, m), 7.3-7.5 (3H, m), 12.06 (1H, s), 14.43 (1H, s) |
| 50 | 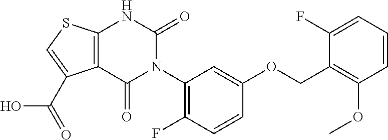 [SMILES: COc1cccc(F)c1COc1ccc(F)c(-n2c(=O)[nH]c3scc(C(=O)O)c3c2=O)c1] | (DMSO-d₆) 3.85 (3H, s), 5.0 (2H, s), 6.88 (1H, t, J = 8.6 Hz), 6.95 (1H, d, J = 8.6 Hz), 7.1-7.2 (1H, m), 7.2-7.25 (1H, m), 7.3-7.4 (1H, m), 7.4-7.5 (1H, m), 7.94 (1H, s), 13.04 (1H, s), 13.93 (1H, s) |
| 51 | 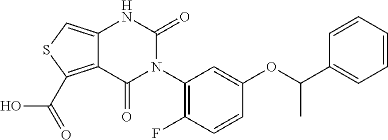 [SMILES: CC(Oc1ccc(F)c(-n2c(=O)[nH]c3csc(C(=O)O)c3c2=O)c1)c1ccccc1] | (DMSO-d₆) 1.5-1.6 (3H, m), 5.4-5.5 (1H, m), 6.95-7.05 (1H, m), 7.1-7.2 (1H, m), 7.2-7.4 (5H, m), 7.4-7.45 (2H, m), 11.95-12.05 (1H, m), 14.42 (1H, s) |
| 52 | 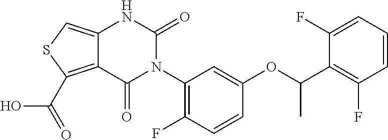 [SMILES: CC(Oc1ccc(F)c(-n2c(=O)[nH]c3csc(C(=O)O)c3c2=O)c1)c1c(F)cccc1F] | (DMSO-d₆) 1.7 (3H, d, J = 6.6 Hz), 5.76 (1H, q, J = 6.6 Hz), 7.0-7.2 (4H, m), 7.25-7.35 (1H, m), 7.35-7.5 (2H, m), 11.95-12.05 (1H, m), 14.42 (1H, s) |
| 53 | 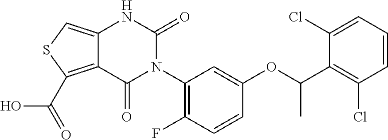 [SMILES: CC(Oc1ccc(F)c(-n2c(=O)[nH]c3csc(C(=O)O)c3c2=O)c1)c1c(Cl)cccc1Cl] | (DMSO-d₆) 1.65-1.75 (3H, m), 6.03 (1H, q, J = 6.6 Hz), 6.85-6.95 (1H, m), 7.05- 7.15 (1H, m), 7.25-7.4 (3H, m), 7.4-7.5 (2H, m), 11.95-12.05 (1H, m), 14.4 (1H, s) |
| 54 | 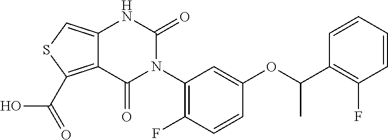 [SMILES: CC(Oc1ccc(F)c(-n2c(=O)[nH]c3csc(C(=O)O)c3c2=O)c1)c1ccccc1F] | (DMSO-d₆) 1.55-1.65 (3H, m), 5.65 (1H, q, J = 6.5 Hz), 7.0-7.05 (1H, m), 7.15- 7.25 (3H, m), 7.25-7.4 (3H, m), 7.45- 7.55 (1H, m), 11.95-12.05 (1H, m), 14.42 (1H, s) |
| 55 | 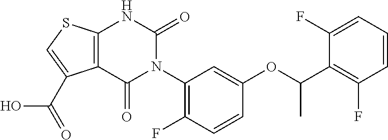 [SMILES: CC(Oc1ccc(F)c(-n2c(=O)[nH]c3scc(C(=O)O)c3c2=O)c1)c1c(F)cccc1F] | (DMSO-d₆) 1.65-1.75 (3H, m), 5.7-5.8 (1H, m), 6.95-7.42 (4H, m), 7.2-7.5 (2H, m), 7.93 (1H, d, J = 7.2 Hz), 12.98 (1H, s), 13.85-14.0 (1H, m) |
| 56 | 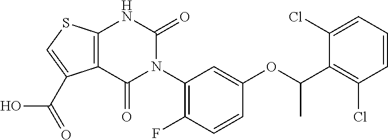 [SMILES: CC(Oc1ccc(F)c(-n2c(=O)[nH]c3scc(C(=O)O)c3c2=O)c1)c1c(Cl)cccc1Cl] | (DMSO-d₆) 1.72 (3H, d, J = 6.6 Hz), 6.0- 6.1 (1H, m), 6.85-6.95 (1H, m), 7.05-7.1 (1H, m), 7.25-7.4 (2H, m), 7.45-7.5 (2H, m), 7.92 (1H, d, J = 11.1 Hz), 12.98 (1H, brs), 13.85-14.0 (1H, m) |
| 57 | 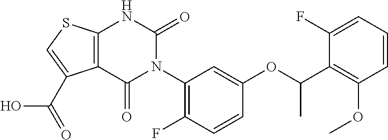 [SMILES: COc1cccc(F)c1C(C)Oc1ccc(F)c(-n2c(=O)[nH]c3scc(C(=O)O)c3c2=O)c1] | (DMSO-d₆) 1.65 (3H, d, J = 6.6 Hz), 3.8- 3.9 (3H, m), 5.79 (1H, q, J = 6.6 Hz), 6.7-6.8 (1H, m), 6.85-6.95 (2H, m), 7.0- 7.1 (1H, m), 7.2-7.35 (2H, m), 7.85-8.0 (1H, m), 12.98 (1H, brs), 13.85-14.05 (1H, m) |
| 58 | 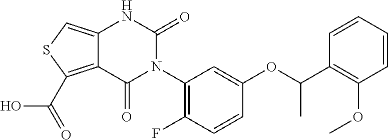 [SMILES: COc1ccccc1C(C)Oc1ccc(F)c(-n2c(=O)[nH]c3csc(C(=O)O)c3c2=O)c1] | (DMSO-d₆) 1.52 (3H, d, J = 6.3 Hz), 3.8- 3.9 (3H, m), 5.64 (1H, q, J = 6.3 Hz), 6.85-7.0 (2H, m), 7.0-7.15 (2H, m), 7.2- 7.4 (4H, m), 11.95-12.0 (1H, m), 14.41 (1H, s) |
| 59 | 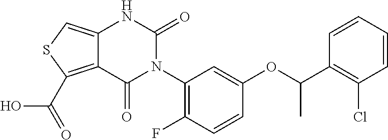 [SMILES: CC(Oc1ccc(F)c(-n2c(=O)[nH]c3csc(C(=O)O)c3c2=O)c1)c1ccccc1Cl] | (DMSO-d₆) 1.45-1.65 (3H, m), 5.55-5.8 (1H, m), 6.8-7.7 (8H, m), 11.98 (1H, s), 14.39 (1H, s) |
| 60 | 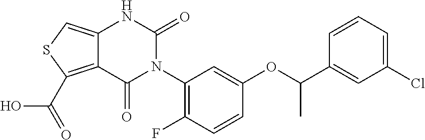 [SMILES: CC(Oc1ccc(F)c(-n2c(=O)[nH]c3csc(C(=O)O)c3c2=O)c1)c1cccc(Cl)c1] | (DMSO-d₆) 1.45-1.65 (3H, m), 5.4-5.6 (1H, m), 6.95-7.6 (8H, m), 11.99 (1H, s), 14.39 (1H, s) |
| 61 | 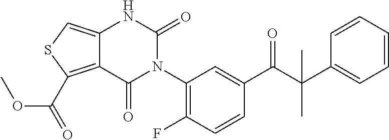 [SMILES: COC(=O)c1scc2[nH]c(=O)n(-c3cc(C(=O)C(C)(C)c4ccccc4)ccc3F)c(=O)c12] | (DMSO-d₆) 1.56 (3H, s), 1.57 (3H, s), 3.82 (3H, s), 7.15-7.45 (8H, m), 7.8-7.9 (1H, m), 11.68 (1H, s) |
| 62 | 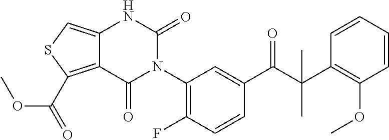 [SMILES: COC(=O)c1scc2[nH]c(=O)n(-c3cc(C(=O)C(C)(C)c4ccccc4OC)ccc3F)c(=O)c12] | (DMSO-d₆) 1.5-1.6 (6H, m), 3.34 (3H, s), 3.82 (3H, s), 6.84 (1H, d, J = 8.2 Hz), 7.0-7.1 (1H, m), 7.1-7.3 (3H, m), 7.3-7.4 (1H, m), 7.5-7.6 (1H, m), 7.8-7.9 (1H, m), 11.63 (1H, s) |
| 63 | 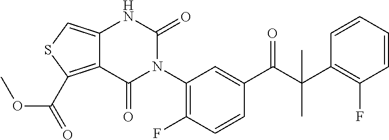 [SMILES: COC(=O)c1scc2[nH]c(=O)n(-c3cc(C(=O)C(C)(C)c4ccccc4F)ccc3F)c(=O)c12] | (DMSO-d₆) 1.59 (3H, s), 1.6 (3H, s), 3.82 (3H, s), 7.05-7.15 (1H, m), 7.19 (1H, s), 7.2-7.4 (3H, m), 7.4-7.5 (1H, m), 7.65-7.75 (1H, m), 7.9-8.0 (1H, m), 11.7 (1H, s) |
| 64 | 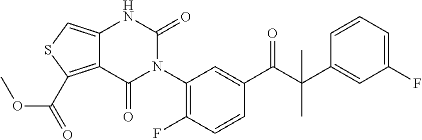 [SMILES: COC(=O)c1scc2[nH]c(=O)n(-c3cc(C(=O)C(C)(C)c4cccc(F)c4)ccc3F)c(=O)c12] | (DMSO-d₆) 1.57 (3H, s), 1.58 (3H, s), 3.82 (3H, s), 7.06 (1H, d, J = 8.4 Hz), 7.1-7.25 (3H, m), 7.25-7.35 (1H, m), 7.35-7.45 (2H, m), 7.8-7.9 (1H, m), 11.68 (1H, s) |
| 65 | 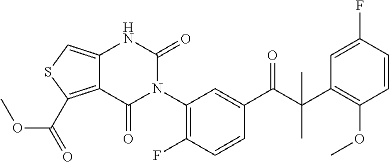 [SMILES: COC(=O)c1scc2[nH]c(=O)n(-c3cc(C(=O)C(C)(C)c4cc(F)ccc4OC)ccc3F)c(=O)c12] | (DMSO-d₆) 1.54 (6H, s), 3.31 (3H, s), 3.82 (3H, s), 6.8-6.9 (1H, m), 7.0-7.1 (1H, m), 7.15-7.25 (2H, m), 7.3-7.45 (2H, m), 7.8-7.9 (1H, m), 11.63 (1H, s) |
| 66 | 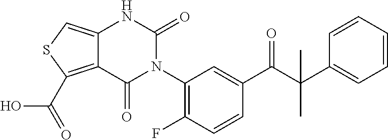 [SMILES: CC(C)(C(=O)c1ccc(F)c(-n2c(=O)[nH]c3csc(C(=O)O)c3c2=O)c1)c1ccccc1] | (DMSO-d₆) 1.55-1.6 (6H, m), 7.25-7.45 (8H, m), 7.9-8.0 (1H, m), 12.0 (1H, s), 14.29 (1H, s) |
| 67 | 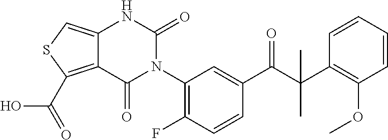 [SMILES: COc1ccccc1C(C)(C)C(=O)c1ccc(F)c(-n2c(=O)[nH]c3csc(C(=O)O)c3c2=O)c1] | (DMSO-d₆) 1.55 (3H, s), 1.56 (3H, s), 3.33 (3H, s), 6.84 (1H, d, J = 8.2 Hz), 7.0-7.1 (1H, m), 7.15-7.3 (2H, m), 7.37 (1H, s), 7.4-7.5 (1H, m), 7.5-7.55 (1H, m), 7.9-7.95 (1H, m), 11.99 (1H, s), 14.35 (1H, s) |
| 68 | 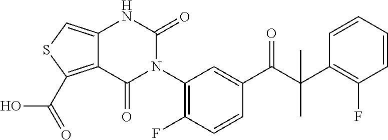 [SMILES: CC(C)(C(=O)c1ccc(F)c(-n2c(=O)[nH]c3csc(C(=O)O)c3c2=O)c1)c1ccccc1F] | (DMSO-d₆) 1.59 (3H, s), 1.62 (3H, s), 7.0-7.1 (1H, m), 7.25-7.4 (4H, m), 7.5- 7.6 (1H, m), 7.65-7.75 (1H, m), 7.95- 8.05 (1H, m), 12.01 (1H, s), 14.29 (1H, s) |
| 69 | 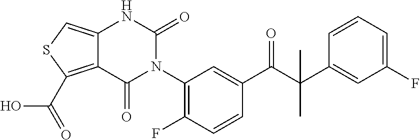 [SMILES: CC(C)(C(=O)c1ccc(F)c(-n2c(=O)[nH]c3csc(C(=O)O)c3c2=O)c1)c1cccc(F)c1] | (DMSO-d₆) 1.58 (3H, s), 1.59 (3H, s), 7.0-7.25 (3H, m), 7.3-7.45 (3H, m), 7.45-7.55 (1H, m), 7.9-7.95 (1H, m), 12.02 (1H, s), 14.29 (1H, s) |
| 70 | 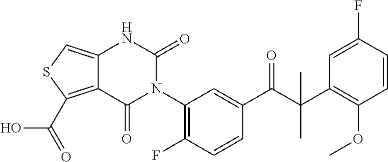 [SMILES: COc1ccc(F)cc1C(C)(C)C(=O)c1ccc(F)c(-n2c(=O)[nH]c3csc(C(=O)O)c3c2=O)c1] | (DMSO-d₆) 1.54 (3H, s), 1.56 (3H, s), 3.31 (3H, s), 6.8-6.9 (1H, m), 7.0-7.1 (1H, m), 7.26 (1H, t, J = 9.2 Mz), 7.3-7.4 (2H, m), 7.5-7.6 (1H, m), 7.85-7.95 (1H, m), 11.99 (1H, s), 14.36 (1H, s) |
| 71 | 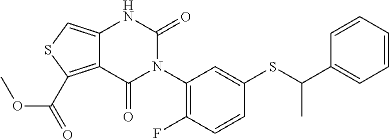 [SMILES: COC(=O)c1scc2[nH]c(=O)n(-c3cc(SC(C)c4ccccc4)ccc3F)c(=O)c12] | (DMSO-d₆) 1.55-1.6 (3H, m), 3.82 (3H, s), 4.6-4.7 (1H, m), 7.1-7.2 (2H, m), 7.2- 7.45 (8H, m), 11.49 (1H, s) |
| 72 | 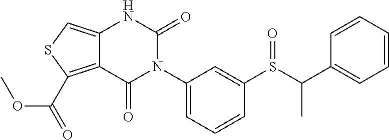 [SMILES: COC(=O)c1scc2[nH]c(=O)n(-c3cccc(S(=O)C(C)c4ccccc4)c3)c(=O)c12] | (DMSO-d₆) 1.36 (3H, d, J = 7.2 Hz), 3.82 (3H, s), 4.05-4.15 (1H, m), 7.15- 7.65 (10H, m), 11.54 (1H, s) |
| 73 | 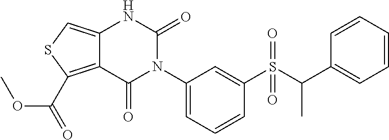 [SMILES: COC(=O)c1scc2[nH]c(=O)n(-c3cccc(S(=O)(=O)C(C)c4ccccc4)c3)c(=O)c12] | (DMSO-d₆) 1.56 (3H, d, J = 7.1 Hz), 3.83 (3H, s), 4.71 (1H, q, J = 7.1 Hz), 7.18 (1H, s), 7.2-7.35 (5H, m), 7.6-7.7 (3H, m), 7.75-7.8 (1H, m), 11.56 (1H, s) |
| 74 | 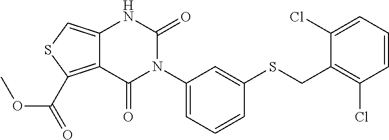 [SMILES: COC(=O)c1scc2[nH]c(=O)n(-c3cccc(SCc4c(Cl)cccc4Cl)c3)c(=O)c12] | (DMSO-d₆) 3.82 (3H, s), 4.44 (2H, s), 7.1-7.25 (2H, m), 7.3-7.4 (1H, m), 7.4- 7.45 (3H, m), 7.45-7.55 (2H, m), 11.5 (1H, s) |
| 75 | 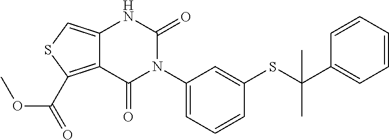 [SMILES: COC(=O)c1scc2[nH]c(=O)n(-c3cccc(SC(C)(C)c4ccccc4)c3)c(=O)c12] | (DMSO-d₆) 1.64 (6H, s), 3.82 (3H, s), 7.05-7.25 (4H, m), 7.25-7.4 (4H, m), 7.4-7.5 (2H, m), 11.45 (1H, s) |
| 76 | 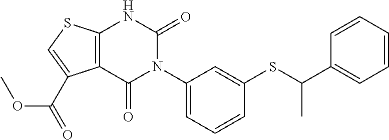 [SMILES: COC(=O)c1csc2[nH]c(=O)n(-c3cccc(SC(C)c4ccccc4)c3)c(=O)c12] | (DMSO-d₆) 1.57 (3H, d, J = 6.9 Hz), 3.74 (3H, s), 4.66 (1H, q, J = 6.9 Hz), 7.1-7.15 (1H, m), 7.2-7.45 (8H, m), 7.59 (1H, s), 12.44 (1H, s) |
| 77 | 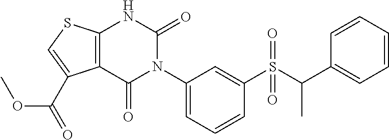 [SMILES: COC(=O)c1csc2[nH]c(=O)n(-c3cccc(S(=O)(=O)C(C)c4ccccc4)c3)c(=O)c12] | (DMSO-d₆) 1.56 (3H, d, J = 6.8 Hz), 3.76 (3H, s), 4.65-4.75 (1H, m), 7.2-7.35 (5H, m), 7.6-7.75 (4H, m), 7.78 (1H, s), 12.52 (1H, s) |
| 78 | 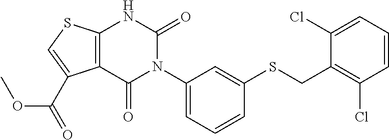 [SMILES: COC(=O)c1csc2[nH]c(=O)n(-c3cccc(SCc4c(Cl)cccc4Cl)c3)c(=O)c12] | (DMSO-d₆) 3.74 (3H, s), 4.44 (2H, s), 7.15-7.25 (1H, m), 7.3-7.55 (6H, m), 7.59 (1H, s), 12.45 (1H, s) |
| 79 | 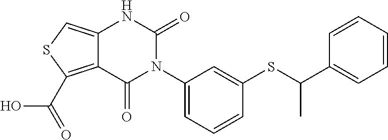 [SMILES: CC(Sc1cccc(-n2c(=O)[nH]c3csc(C(=O)O)c3c2=O)c1)c1ccccc1] | (DMSO-d₆) 1.58 (3H, d, J = 6.9 Hz), 4.65 (1H, q, J = 6.9 Hz), 7.2-7.5 (10H, m), 11.93 (1H, s), 14.87 (1H, s) |
| 80 | 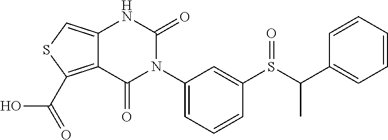 [SMILES: CC(c1ccccc1)S(=O)c1cccc(-n2c(=O)[nH]c3csc(C(=O)O)c3c2=O)c1] | (DMSO-d₆) 1.39 (3H, d, J = 7.3 Hz), 4.05-4.15 (1H, m), 7.2-7.8 (10H, m), 11.95 (1H, s), 14.8 (1H, s) |
| 81 | 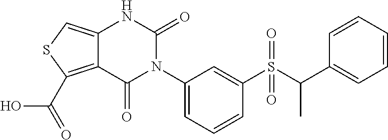 [SMILES: CC(c1ccccc1)S(=O)(=O)c1cccc(-n2c(=O)[nH]c3csc(C(=O)O)c3c2=O)c1] | (DMSO-d₆) 1.59 (3H, d, J = 7.7 Hz), 4.72 (1H, q, J = 7.7 Hz), 7.2-7.35 (5H, m), 7.39 (1H, s), 7.65-7.9 (4H, m), 11.96 (1H, s), 14.73 (1H, s) |
| 82 | 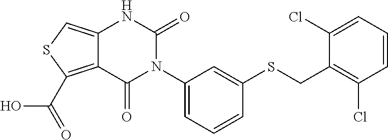 [SMILES: O=C(O)c1scc2[nH]c(=O)n(-c3cccc(SCc4c(Cl)cccc4Cl)c3)c(=O)c12] | (DMSO-d₆) 4.44 (2H, s), 7.25-7.45 (3H, m), 7.45-7.55 (5H, m), 11.93 (1H, s), 14.87 (1H, s) |
| 83 | 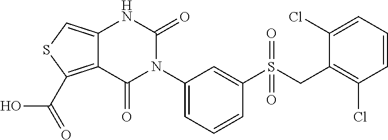 [SMILES: O=C(O)c1scc2[nH]c(=O)n(-c3cccc(S(=O)(=O)Cc4c(Cl)cccc4Cl)c3)c(=O)c12] | (DMSO-d₆) 4.94 (2H, s), 7.35-7.55 (4H, m), 7.75-7.85 (3H, m), 7.95-8.0 (1H, m), 11.96 (1H, s), 14.75 (1H, s) |
| 84 | 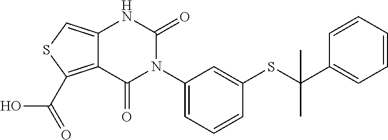 [SMILES: CC(C)(Sc1cccc(-n2c(=O)[nH]c3csc(C(=O)O)c3c2=O)c1)c1ccccc1] | (DMSO-d₆) 1.65 (6H, s), 7.1-7.5 (10H, m), 11.88 (1H, s), 14.84 (1H, s) |
| 85 | 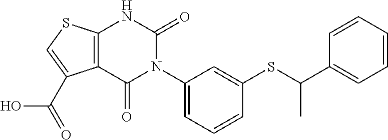 [SMILES: CC(Sc1cccc(-n2c(=O)[nH]c3scc(C(=O)O)c3c2=O)c1)c1ccccc1] | (DMSO-d₆) 1.58 (3H, d, J = 7.0 Hz), 4.66 (1H, q, J = 7.0 Hz), 7.15-7.45 (9H, m), 8.0 (1H, s), 12.94 (1H, s), 14.43 (1H, s) |
| 86 | 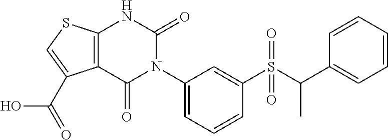 [SMILES: CC(c1ccccc1)S(=O)(=O)c1cccc(-n2c(=O)[nH]c3scc(C(=O)O)c3c2=O)c1] | (DMSO-d₆) 1.58 (3H, d, J = 7.0 Hz), 4.72 (1H, q, J = 7.0 Hz), 7.2-7.35 (5H, m), 7.65-7.8 (3H, m), 7.84 (1H, s), 7.98 (1H, s), 12.96 (1H, s), 14.26 (1H, s) |
| 87 | 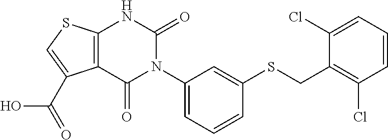 [SMILES: O=C(O)c1csc2[nH]c(=O)n(-c3cccc(SCc4c(Cl)cccc4Cl)c3)c(=O)c12] | (DMSO-d₆) 4.45 (2H, s), 7.25-7.3 (1H, m), 7.3-7.4 (1H, m), 7.4-7.55 (5H, m), 8.0 (1H, s), 12.94 (1H, s), 14.42 (1H, s) |
| 88 | 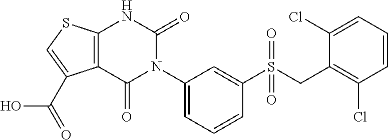 [SMILES: O=C(O)c1csc2[nH]c(=O)n(-c3cccc(S(=O)(=O)Cc4c(Cl)cccc4Cl)c3)c(=O)c12] | (DMSO-d₆) 4.94 (2H, s), 7.35-7.45 (1H, m), 7.45-7.55 (2H, m), 7.7-7.85 (3H, m), 7.95-8.0 (2H, m), 12.96 (1H, s), 14.3 (1H, s) |
| 89 | 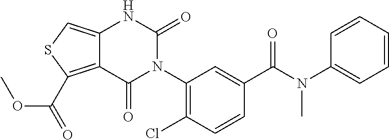 [SMILES: COC(=O)c1scc2[nH]c(=O)n(-c3cc(C(=O)N(C)c4ccccc4)ccc3Cl)c(=O)c12] | (DMSO-d₆) 3.37 (3H, s), 3.83 (3H, s), 7.15-7.25 (5H, m), 7.25-7.35 (2H, m), 7.44 (1H, d, J = 8.1 Hz), 7.56 (1H, d, J = 1.9 Hz), 11.63 (1H, s) |
| 90 | 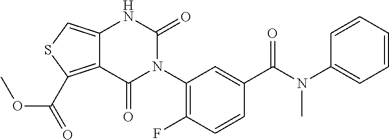 [SMILES: COC(=O)c1scc2[nH]c(=O)n(-c3cc(C(=O)N(C)c4ccccc4)ccc3F)c(=O)c12] | (DMSO-d₆) 3.37 (3H, s), 3.83 (3H, s), 7.15-7.35 (8H, m), 7.53 (1H, dd, J = 7.3 Hz, 1.9 Hz), 11.63 (1H, s) |
| 91 | 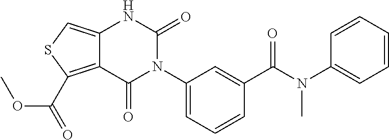 [SMILES: COC(=O)c1scc2[nH]c(=O)n(-c3cccc(C(=O)N(C)c4ccccc4)c3)c(=O)c12] | (DMSO-d₆) 3.37 (3H, s), 3.82 (3H, s), 7.1-7.4 10H, m), 11.46 (1H, s) |
| 92 | 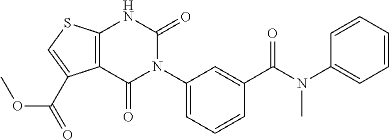 [SMILES: COC(=O)c1csc2[nH]c(=O)n(-c3cccc(C(=O)N(C)c4ccccc4)c3)c(=O)c12] | (DMSO-d₆) 3.37 (3H, s), 3.74 (3H, s), 7.1-7.4 (9H, m), 7.58 (1H, s), 12.42 (1H, s) |
| 93 | 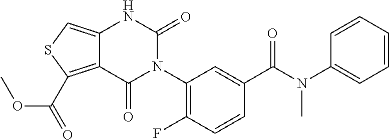 [SMILES: COC(=O)c1scc2[nH]c(=O)n(-c3cc(C(=O)N(C)c4ccccc4)ccc3F)c(=O)c12] | (DMSO-d₆) 3.38 (3H, s), 7.15-7.4 (8H, m), 7.5-7.6 (1H, m), 11.96 (1H, s), 14.34 (1H, s) |
| 94 | 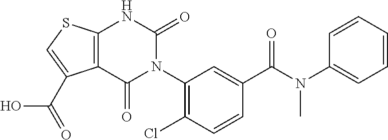 [SMILES: CN(C(=O)c1ccc(Cl)c(-n2c(=O)[nH]c3scc(C(=O)O)c3c2=O)c1)c1ccccc1] | (DMSO-d₆) 3.37 (3H, s), 7.15-7.4 (7H, m), 7.44 (1H, d, J = 8.7 Hz), 7.72 (1H, s) |
| 95 | 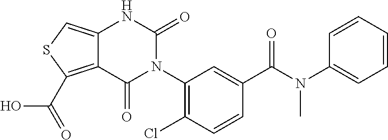 [SMILES: CN(C(=O)c1ccc(Cl)c(-n2c(=O)[nH]c3csc(C(=O)O)c3c2=O)c1)c1ccccc1] | (DMSO-d₆) 3.38 (3H, s), 7.15-7.25 (3H, m), 7.25-7.35 (3H, m), 7.38 (1H, s), 7.5 (1H, d, J = 7.5 Hz), 7.58 (1H, d, J = 1.9 Hz), 11.98 (1H, s), 14.33 (1H, s) |
| 96 | 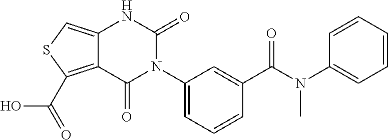 [SMILES: CN(C(=O)c1cccc(-n2c(=O)[nH]c3csc(C(=O)O)c3c2=O)c1)c1ccccc1] | (DMSO-d₆) 3.37 (3H, s), 7.15-7.4 (10H, m), 11.89 (1H, s), 14.81 (1H, s) |
| 97 | 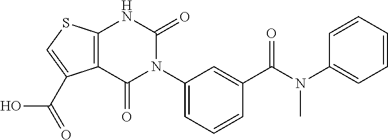 [SMILES: CN(C(=O)c1cccc(-n2c(=O)[nH]c3scc(C(=O)O)c3c2=O)c1)c1ccccc1] | (DMSO-d₆) 3.37 (3H, s), 7.15-7.4 (9H, m), 7.99 (1H, s), 12.9 (1H, s), 14.37 (1H, s) |
| 98 | 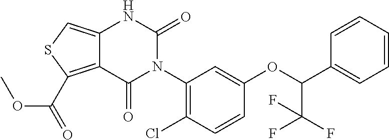 [SMILES: COC(=O)c1scc2[nH]c(=O)n(-c3cc(OC(c4ccccc4)C(F)(F)F)ccc3Cl)c(=O)c12] | (DMSO-d₆) 3.75-3.85 (3H, m), 6.3-6.4 (1H, m), 7.05-7.15 (1H, m), 7.2 (1H, s), 7.32 (1H, dd, J = 7.6 Hz, 2.9 Hz), 7.45- 7.55 (4H, m), 7.55-7.65 (2H, m), 11.66 (1H, s) |
| 99 | 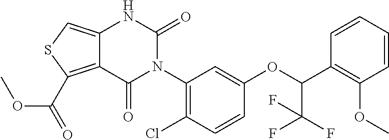 | (DMSO-d₆) 3.75-3.95 (6H, m), 6.2-6.35 (1H, m), 6.9-7.0 (1H, m), 7.0-7.1 (1H, m), 7.17 (1H, d, J = 8.0 Hz), 7.21 (1H, s), 7.31 (1H, dd, J = 6.4 Hz, 3.0 Hz), 7.4-7.55 (3H, m), 11.64 (1H, s) |
| 100 | 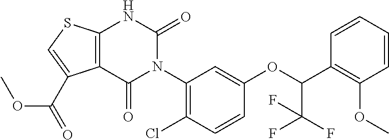 | (DMSO-d₆) 3.7-3.75 (3H, m), 3.85-3.95 (3H, m), 6.2-6.35 (1H, m), 6.9-7.0 (1H, m), 7.04 (1H, t, J = 7.6 Hz), 7.15-7.2 (1H, m), 7.25-7.35 (1H, m), 7.4-7.55 (3H, m), 7.64 (1H, s), 12.6 (1H, s) |
| 101 | 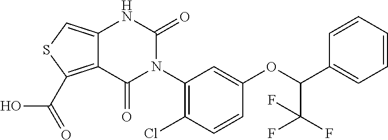 [SMILES: O=C(O)c1scc2[nH]c(=O)n(-c3cc(OC(c4ccccc4)C(F)(F)F)ccc3Cl)c(=O)c12] | (DMSO-d₆) 6.3-6.4 (1H, m), 7.1-7.2 (1H, m), 7.35-7.4 (2H, m), 7.45-7.55 (3H, m), 7.55-7.65 (3H, m), 11.95-12.1 (1H, m), 14.32 (1H, s) |
| 102 | 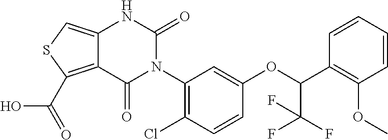 [SMILES: COc1ccccc1C(Oc1ccc(Cl)c(-n2c(=O)[nH]c3csc(C(=O)O)c3c2=O)c1)C(F)(F)F] | (DMSO-d₆) 3.85-3.95 (3H, m), 6.25-6.35 (1H, m), 6.95-7.1 (2H, m), 7.15-7.2 (1H, m), 7.35-7.5 (4H, m), 7.56 (1H, d, J = 8.9 Hz), 12.0-12.1 (1H, m), 14.34 (1H, s) |
| 103 | 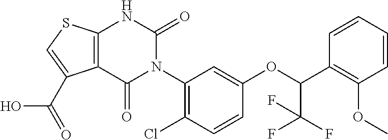 [SMILES: COc1ccccc1C(Oc1ccc(Cl)c(-n2c(=O)[nH]c3scc(C(=O)O)c3c2=O)c1)C(F)(F)F] | (DMSO-d₆) 3.85-3.95 (3H, m), 6.25-6.35 (1H, m), 6.95-7.1 (2H, m), 7.15-7.2 (1H, m), 7.3-7.4 (1H, m), 7.4-7.5 (2H, m), 7.55 (1H, d, J = 9.0 Hz), 7.92 (1H, d, J = 4.5 Hz), 13.0 (1H, brs), 13.8-13.95 (1H, m) |
| 104 | 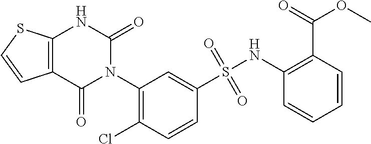 [SMILES: COC(=O)c1ccccc1NS(=O)(=O)c1ccc(Cl)c(-n2c(=O)[nH]c3sccc3c2=O)c1] | (CDCl3) 3.89 (3H, s), 6.84 (1H, d, J = 5.8 Hz), 7.05-7.1 (1H, m), 7.28 (1H, d, J = 5.8 Hz), 7.45-7.55 (1H, m), 7.59 (1H, d, J = 8.5 Hz), 7.65-7.75 (1H, m), 7.8- 7.85 (1H, m), 7.85-8.0 (2H, m), 10.06 (1H, s), 10.75 (1H, s) |
| 105 | 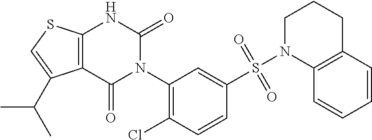 [SMILES: CC(C)c1csc2[nH]c(=O)n(-c3cc(S(=O)(=O)N4CCCc5ccccc54)ccc3Cl)c(=O)c12] | (CDCl3) 1.6-1.75 (2H, m), 2.45-2.55 (2H, m), 3.45-3.6 (1H, m), 3.7-3.85 (2H, m), 6.45 (1H, d, J = 0.5 Hz), 7.01 (1H, d, J = 7.0 Hz), 7.05-7.25 (2H, m), 7.5-7.6 (3H, m), 7.76 (1H, d, J = 7.7 Hz), 10.68 (1H, s) |
| 106 | 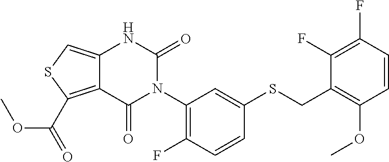 [SMILES: COC(=O)c1scc2[nH]c(=O)n(-c3cc(SCc4c(OC)ccc(F)c4F)ccc3F)c(=O)c12] | (DMSO-d6) 3.74 (3H, s), 3.83 (3H, s), 4.15-4.2 (2H, m), 6.75-6.85 (1H, m), 7.21 (1H, s), 7.25-7.4 (2H, m), 7.4-7.5 (1H, m), 7.5-7.55 (1H, m), 11.65 (1H, s) |
| 107 | 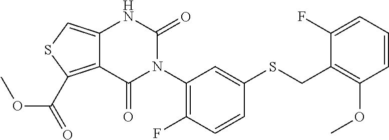 [SMILES: COC(=O)c1scc2[nH]c(=O)n(-c3cc(SCc4c(F)cccc4OC)ccc3F)c(=O)c12] | (DMSO-d6) 3.77 (3H, s), 3.83 (3H, s), 4.15 (2H, s), 6.75-6.9 (2H, m), 7.15-7.55 (5H, m), 11.65 (1H, s) |
| 108 | 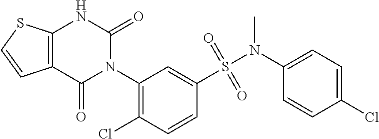 [SMILES: CN(c1ccc(Cl)cc1)S(=O)(=O)c1ccc(Cl)c(-n2c(=O)[nH]c3sccc3c2=O)c1] | (CDCl3) 3.16 (3H, s), 6.88 (1H, d, J = 5.8 Hz), 7.0-7.1 (2H, m), 7.25-7.35 (3H, m), 7.5-7.6 (2H, m), 7.6-7.7 (1H, m), 9.5-11.0 (1H, br) |
| 109 | 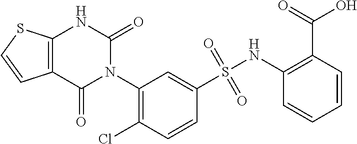 [SMILES: O=C(O)c1ccccc1NS(=O)(=O)c1ccc(Cl)c(-n2c(=O)[nH]c3sccc3c2=O)c1] | (DMSO-d6) 7.1-7.25 (3H, m), 7.44 (1H, d, J = 8.5 Hz), 7.5-7.6 (1H, m), 7.87 (1H, d, J = 8.5 Hz), 7.9-8.0 (2H, m), 8.18 (1H, d, J = 2.2 Hz), 11.0-12.0 (1H, br), 12.52 (1H, s) |
| 110 | 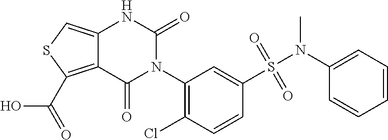 [SMILES: CN(c1ccccc1)S(=O)(=O)c1ccc(Cl)c(-n2c(=O)[nH]c3csc(C(=O)O)c3c2=O)c1] | (CDCl3) 3.22 (3H, s), 7.05-7.15 (3H, m), 7.25-7.35 (3H, m), 7.45-7.55 (1H, m), 7.71 (1H, s), 9.22 (1H, s), 14.14 (1H, s) |
| 111 | 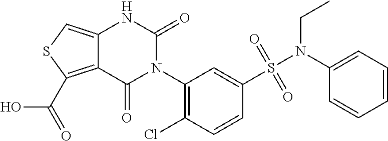 [SMILES: CCN(c1ccccc1)S(=O)(=O)c1ccc(Cl)c(-n2c(=O)[nH]c3csc(C(=O)O)c3c2=O)c1] | (CDCl3) 1.11 (3H, t, J = 7.1 Hz), 3.5-3.6 (1H, m), 3.65-3.8 (1H, m), 7.05-7.1 (2H, m), 7.13 (1H, s), 7.25-7.35 (3H, m), 7.55 (1H, d, J = 2.2 Hz), 7.7 (1H, d, J = 8.4 Hz), 7.77 (1H, dd, J = 8.4 Hz, 2.2 Hz), 9.22 (1H, s), 14.17 (1H, s) |
| 112 | 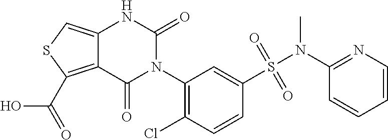 [SMILES: CN(c1ccccn1)S(=O)(=O)c1ccc(Cl)c(-n2c(=O)[nH]c3csc(C(=O)O)c3c2=O)c1] | (CDCl3) 3.33 (3H, s), 7.1 (1H, s), 7.15- 7.2 (1H, m), 7.57 (1H, d, J = 8.2 Hz), 7.6-7.75 (4H, m), 8.3-8.4 (1H, m), 9.05 (1H, s), 14.09 (1H, s) |
| 113 | 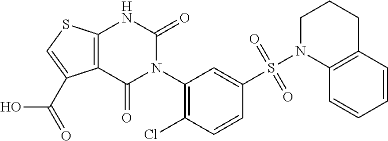 [SMILES: O=C(O)c1csc2[nH]c(=O)n(-c3cc(S(=O)(=O)N4CCCc5ccccc54)ccc3Cl)c(=O)c12] | (DMSO-d6) 1.6-1.75 (2H, m), 2.45-2.55 (2H, m), 3.7-3.85 (2H, m), 6.9-7.0 (1H, m), 7.05-7.25 (3H, m), 7.5-7.6 (2H, m), 7.78 (1H, d, J = 8.5 Hz), 8.0-8.05 (1H, m), 8.5-8.55 (1H, m), 11.52 (1H, s) |
| 114 | 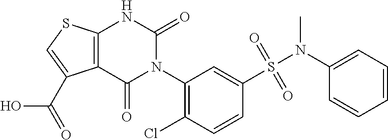 [SMILES: CN(c1ccccc1)S(=O)(=O)c1ccc(Cl)c(-n2c(=O)[nH]c3scc(C(=O)O)c3c2=O)c1] | (DMSO-d6) 3.17 (3H, s), 7.05-7.15 (2H, m), 7.25-7.4 (3H, m), 7.63 (1H, dd, J = 8.6 Hz, 2.2 Hz), 7.85-7.95 (3H, m), 12.5- 13.5 (1H, br), 13.79 (1H, s) |
| 115 | 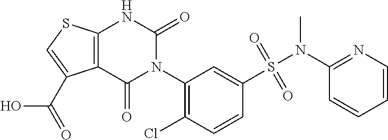 [SMILES: CN(c1ccccn1)S(=O)(=O)c1ccc(Cl)c(-n2c(=O)[nH]c3scc(C(=O)O)c3c2=O)c1] | (DMSO-d6) 3.26 (3H, s), 7.25-7.3 (1H, m), 7.45-7.55 (1H, m), 7.65-7.75 (1H, m), 7.8-7.95 (3H, m), 8.05-8.1 (1H, m), 8.3-8.4 (1H, m) |
| 116 | 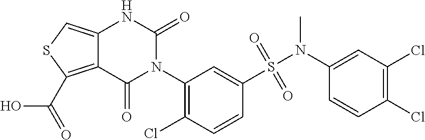 [SMILES: CN(c1ccc(Cl)c(Cl)c1)S(=O)(=O)c1ccc(Cl)c(-n2c(=O)[nH]c3csc(C(=O)O)c3c2=O)c1] | (CDCl3) 3.17 (3H, s), 6.9-7.0 (1H, m), 7.13 (1H, s), 7.29 (1H, d, J = 2.5 Hz), 7.39 (1H, d, J = 8.4 Hz), 7.51 (1H, d, J = 1.2 Hz), 7.7-7.8 (2H, m), 9.12 (1H, s), 14.05 (1H, s) |
| 117 | 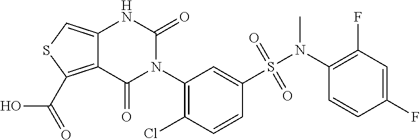 [SMILES: CN(c1ccc(F)cc1F)S(=O)(=O)c1ccc(Cl)c(-n2c(=O)[nH]c3csc(C(=O)O)c3c2=O)c1] | (CDCl3) 3.25 (3H, s), 6.75-6.9 (2H, m), 7.13 (1H, s), 7.2-7.3 (1H, m), 7.63 (1H, d, J = 2.1 Hz), 7.75 (1H, d, J = 8.6 Hz), 7.8-7.85 (1H, m), 9.03 (1H, s), 14.11 (1H, s) |
| 118 | 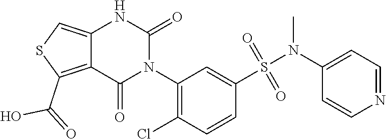 [SMILES: CN(c1ccncc1)S(=O)(=O)c1ccc(Cl)c(-n2c(=O)[nH]c3csc(C(=O)O)c3c2=O)c1] | (CDCl3) 3.22 (3H, s), 7.08 (1H, s), 7.15- 7.25 (2H, m), 7.38 (1H, d, J = 1.7 Hz), 7.7-7.8 (2H, m), 8.55-8.65 (2H, m) |
| 119 | 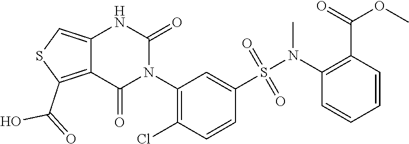 [SMILES: COC(=O)c1ccccc1N(C)S(=O)(=O)c1ccc(Cl)c(-n2c(=O)[nH]c3csc(C(=O)O)c3c2=O)c1] | (DMSO-d6) 3.32 (3H, s), 3.8 (3H, s), 6.9-6.95 (1H, m), 7.35-7.55 (3H, m), 7.7-7.85 (2H, m), 7.9-8.0 (2H, m), 12.03 (1H, s), 14.29 (1H, s) |
| 120 | 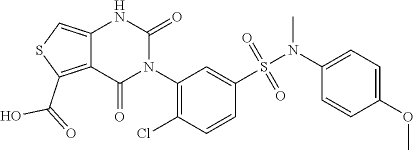 [SMILES: COc1ccc(N(C)S(=O)(=O)c2ccc(Cl)c(-n3c(=O)[nH]c4csc(C(=O)O)c4c3=O)c2)cc1] | (DMSO-d6) 3.12 (3H, s), 3.73 (3H, s), 6.85-6.9 (2H, m), 6.95-7.0 (2H, m), 7.39 (1H, s), 7.68 (1H, dd, J = 8.4 Hz, 2.3 Hz), 7.85-7.95 (2H, m), 12.03 (1H, s), 14.3 (1H, s) |
| 121 | 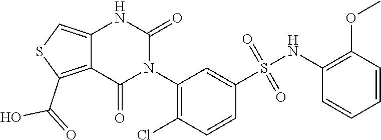 [SMILES: COc1ccccc1NS(=O)(=O)c1ccc(Cl)c(-n2c(=O)[nH]c3csc(C(=O)O)c3c2=O)c1] | (DMSO-d6) 3.51 (3H, s), 6.85-6.95 (2H, m), 7.15-7.25 (2H, m), 7.38 (1H, s), 7.7- 7.8 (1H, m), 7.85-7.95 (1H, m), 7.95-8.0 (1H, m), 9.74 (1H, s), 12.02 (1H, s), 13.5-15.0 (1H, br) |
| 122 | 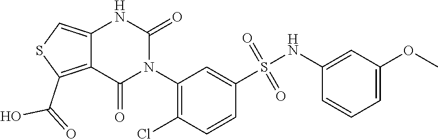 [SMILES: COc1cccc(NS(=O)(=O)c2ccc(Cl)c(-n3c(=O)[nH]c4csc(C(=O)O)c4c3=O)c2)c1] | (DMSO-d6) 3.67 (3H, s), 6.6-6.75 (3H, m), 7.1-7.2 (1H, m), 7.38 (1H, s), 7.84 (1H, dd, J = 8.5 Hz, 2.2 Hz), 7.89 (1H, d, J = 8.5 Hz), 8.13 (1H, d, J = 2.2 Hz), 10.49 (1H, s), 12.04 (1H, s), 14.22 (1H, s) |
| 123 | 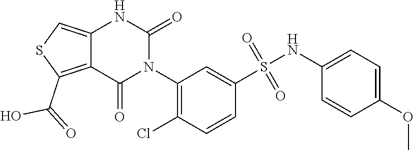 [SMILES: COc1ccc(NS(=O)(=O)c2ccc(Cl)c(-n3c(=O)[nH]c4csc(C(=O)O)c4c3=O)c2)cc1] | (DMSO-d6) 3.69 (3H, s), 6.83 (2H, d, J = 8.9 Hz), 6.99 (2H, d, J = 8.9 Hz), 7.38 (1H, s), 7.75 (1H, dd, J = 8.5 Hz, 2.0 Hz), 7.88 (1H, d, J = 8.5 Hz), 7.99 (1H, d, J = 2.0 Hz), 10.08 (1H, s), 12.03 (1H, s), 14.24 (1H, s) |
| 124 | 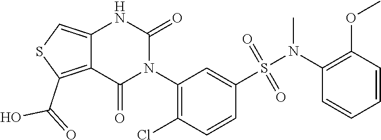 [SMILES: COc1ccccc1N(C)S(=O)(=O)c1ccc(Cl)c(-n2c(=O)[nH]c3csc(C(=O)O)c3c2=O)c1] | (DMSO-d6) 3.17 (3H, s), 3.47 (3H, s), 6.9-7.0 (2H, m), 7.15-7.2 (1H, m), 7.25- 7.35 (1H, m), 7.39 (1H, s), 7.71 (1H, dd, J = 8.6 Hz, 2.2 Hz), 7.9 (1H, d, J = 8.6 Hz), 8.02 (1H, d, J = 2.2 Hz), 12.02 (1H, s), 14.31 (1H, s) |
| 125 | 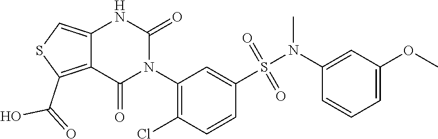 [SMILES: COc1cccc(N(C)S(=O)(=O)c2ccc(Cl)c(-n3c(=O)[nH]c4csc(C(=O)O)c4c3=O)c2)c1] | (DMSO-d6) 3.18 (3H, s), 3.69 (3H, s), 6.55-6.7 (2H, m), 6.8-6.9 (1H, m), 7.2- 7.3 (1H, m), 7.35-7.4 (1H, m), 7.6-7.7 (1H, m), 7.85-8.05 (2H, m), 12.04 (1H, s), 14.26 (1H, s) |
| 126 | 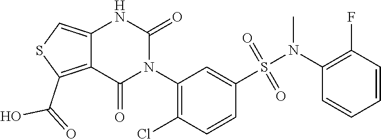 [SMILES: CN(c1ccccc1F)S(=O)(=O)c1ccc(Cl)c(-n2c(=O)[nH]c3csc(C(=O)O)c3c2=O)c1] | (DMSO-d6) 3.18 (3H, s), 7.1-7.35 (3H, m), 7.35-7.45 (2H, m), 7.81 (1H, d, J = 2.3 Hz), 7.95 (1H, d, J = 8.5 Hz), 8.03 (1H, d, J = 2.3 Hz), 12.03 (1H, s), 14.92 (1H, s) |
| 127 | 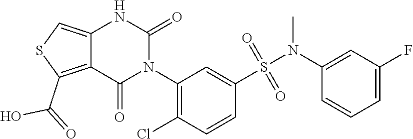 [SMILES: CN(c1cccc(F)c1)S(=O)(=O)c1ccc(Cl)c(-n2c(=O)[nH]c3csc(C(=O)O)c3c2=O)c1] | (DMSO-d6) 3.19 (3H, s), 6.95-7.05 (2H, m), 7.1-7.2 (1H, m), 7.35-7.45 (2H, m), 7.7-7.75 (1H, m), 7.9-8.0 (2H, m), 12.04 (1H, s), 14.27 (1H, s) |
| 128 | 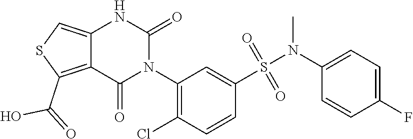 [SMILES: CN(c1ccc(F)cc1)S(=O)(=O)c1ccc(Cl)c(-n2c(=O)[nH]c3csc(C(=O)O)c3c2=O)c1] | (DMSO-d6) 3.15 (3H, s), 7.05-7.25 (4H, m), 7.38 (1H, s), 7.69 (1H, dd, J = 8.5 Hz, 2.5 Hz), 7.9 (1H, d, J = 2.5 Hz), 7.93 (1H, d, J = 8.5 Hz), 12.05 (1H, s), 14.29 (1H, s) |
| 129 | 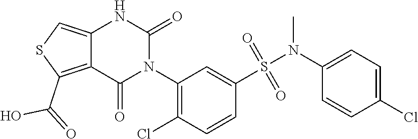 [SMILES: CN(c1ccc(Cl)cc1)S(=O)(=O)c1ccc(Cl)c(-n2c(=O)[nH]c3csc(C(=O)O)c3c2=O)c1] | (DMSO-d6) 3.15 (3H, s), 7.1-7.5 (2H, m), 7.35-7.45 (3H, m), 7.7 (1H, dd, J = 8.6 Hz, 2.1 Hz), 7.9-7.95 (2H, m), 12.05 (1H, s), 14.28 (1H, s) |
| 130 | 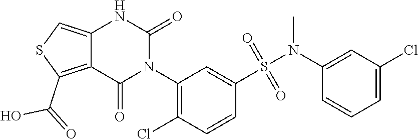 [SMILES: CN(c1cccc(Cl)c1)S(=O)(=O)c1ccc(Cl)c(-n2c(=O)[nH]c3csc(C(=O)O)c3c2=O)c1] | (DMSO-d6) 3.19 (3H, s), 7.05-7.1 (1H, m), 7.2-7.25 (1H, m), 7.35-7.45 (3H, m), 7.72 (1H, dd, J = 8.5 Hz, 2.1 Hz), 7.9- 8.0 (2H, m), 12.06 (1H, s), 14.3 (1H, s) |
| 131 | 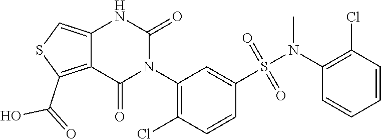 [SMILES: CN(c1ccccc1Cl)S(=O)(=O)c1ccc(Cl)c(-n2c(=O)[nH]c3csc(C(=O)O)c3c2=O)c1] | (DMSO-d6) 3.16 (3H, s), 7.0-7.1 (1H, m), 7.25-7.45 (3H, m), 7.55-7.6 (1H, m), 7.85-7.9 (1H, m), 7.97 (1H, d, J = 8.5 Hz), 8.1 (1H, s), 12.06 (1H, s), 14.31 (1H, s) |
| 132 | 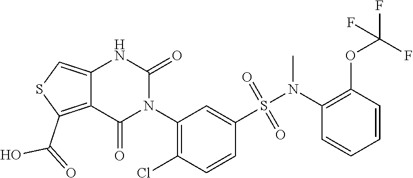 [SMILES: CN(c1ccccc1OC(F)(F)F)S(=O)(=O)c1ccc(Cl)c(-n2c(=O)[nH]c3csc(C(=O)O)c3c2=O)c1] | (DMSO-d6) 3.13 (3H, s), 7.0-7.1 (1H, m), 7.3-7.4 (2H, m), 7.45-7.55 (2H, m), 7.86 (1H, dd, J = 8.5 Hz, 2.4 Hz), 7.98 (1H, d, J = 8.5 Hz), 8.02 (1H, d, J = 2.4 Hz), 12.03 (1H, s), 14.3 (1H, s) |
| 133 | 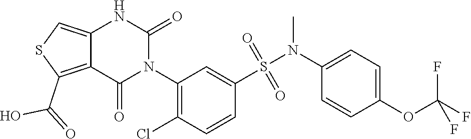 [SMILES: CN(c1ccc(OC(F)(F)F)cc1)S(=O)(=O)c1ccc(Cl)c(-n2c(=O)[nH]c3csc(C(=O)O)c3c2=O)c1] | (DMSO-d6) 3.17 (3H, s), 7.2-7.3 (2H, m), 7.3-7.4 (3H, m), 7.71 (1H, dd, J = 8.5 Hz, 2.1 Hz), 7.9 (1H, d, J = 2.1 Hz), 7.93 (1H, d, J = 8.5 Hz), 12.0 (1H, s), 14.25 (1H, s) |
| 134 | 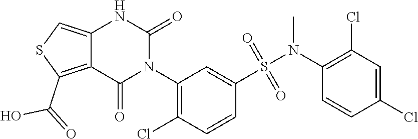 [SMILES: CN(c1ccc(Cl)cc1Cl)S(=O)(=O)c1ccc(Cl)c(-n2c(=O)[nH]c3csc(C(=O)O)c3c2=O)c1] | (DMSO-d6) 3.15 (3H, s), 7.06 (1H, d, J = 8.5 Hz), 7.35-7.45 (2H, m), 7.76 (1H, d, J = 2.4 Hz), 7.85-7.9 (1H, m), 7.97 (1H, d, J = 8.54 Hz), 8.07 (1H, d, J = 2.2 Hz), 12.03 (1H, s), 14.27 (1H, s) |
| 135 | 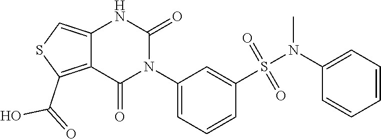 [SMILES: CN(c1ccccc1)S(=O)(=O)c1cccc(-n2c(=O)[nH]c3csc(C(=O)O)c3c2=O)c1] | (DMSO-d6) 3.16 (3H, s), 7.05-7.1 (2H, m), 7.25-7.4 (4H, m), 7.55-7.65 (1H, m), 7.7-7.8 (3H, m), 11.95 (1H, s), 14.77 (1H, s) |
| 136 | 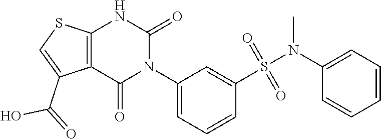 [SMILES: CN(c1ccccc1)S(=O)(=O)c1cccc(-n2c(=O)[nH]c3scc(C(=O)O)c3c2=O)c1] | (DMSO-d6) 3.16 (3H, 2), 7.05-7.1 (2H, m), 7.25-7.4 (3H, m), 7.55-7.65 (1H, m), 7.7-7.8 (3H, m), 7.98 (1H, s), 12.95 (1H, s), 14.31 (1H, s) |
| 137 | 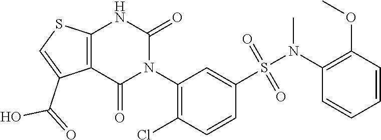 [SMILES: COc1ccccc1N(C)S(=O)(=O)c1ccc(Cl)c(-n2c(=O)[nH]c3scc(C(=O)O)c3c2=O)c1] | (DMSO-d6) 3.16 (3H, s), 3.47 (3H, s), 6.85-7.0 (2H, m), 7.1-7.2 (1H, m), 7.25- 7.35 (1H, m), 7.69 (1H, dd, J = 8.4 Hz, 2.1 Hz), 7.85-7.95 (2H, m), 7.99 (1H, d, J = 2.1 Hz), 12.5-13.5 (1H, br), 13.83 (1H, brs) |
| 138 | 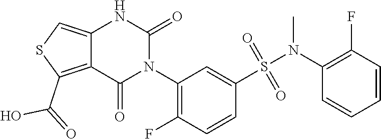 [SMILES: CN(c1ccccc1F)S(=O)(=O)c1ccc(F)c(-n2c(=O)[nH]c3csc(C(=O)O)c3c2=O)c1] | (DMSO-d6) 3.17 (3H, s), 7.1-7.25 (2H, m), 7.25-7.35 (1H, m), 7.35-7.45 (2H, m), 7.65-7.75 (1H, m), 7.8-7.9 (1H, m), 8.02 (1H, dd, J = 6.6 Hz, 2.3 Hz), 12.03 (1H, s), 14.31 (1H, s) |
| 139 | 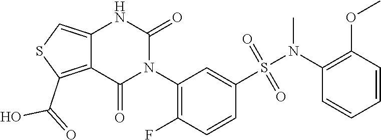 [SMILES: COc1ccccc1N(C)S(=O)(=O)c1ccc(F)c(-n2c(=O)[nH]c3csc(C(=O)O)c3c2=O)c1] | (DMSO-d6) 3.16 (3H, s), 3.47 (3H, s), 6.9-7.0 (2H, m), 7.19 (1H, dd, J = 8.0 Hz, 1.5 Hz), 7.3-7.35 (1H, m), 7.37 (1H, s), 7.6-7.7 (1H, m), 7.7-7.8 (1H, m), 8.0 (1H, dd, J = 6.6 Hz, 2.5 Hz), 12.02 (1H, s), 14.32 (1H, s) |
| 140 | 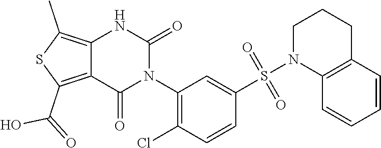 [SMILES: Cc1sc(C(=O)O)c2c(=O)n(-c3cc(S(=O)(=O)N4CCCc5ccccc54)ccc3Cl)c(=O)[nH]c12] | (DMSO-d6) 1.6-1.7 (2H, m), 2.45-2.55 (5H, m), 3.7-3.8 (2H, m), 7.05-7.25 (3H, m), 7.56 (1H, d, J = 8.5 Hz), 7.6-7.65 (1H, m), 7.84 (1H, d, J = 8.5 Hz), 8.1 (1H, d, J = 2.4 Hz), 11.94 (1H, s), 14.22 (1H, s) |
| 141 | 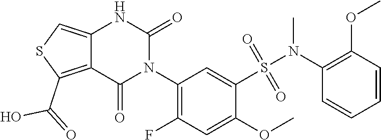 [SMILES: COc1ccccc1N(C)S(=O)(=O)c1cc(-n2c(=O)[nH]c3csc(C(=O)O)c3c2=O)c(F)cc1OC] | (CD3OD) 3.36 (3H, s), 3.54 (3H, s), 4.01 (3H, s), 6.85-6.95 (2H, m), 7.15- 7.35 (4H, m), 7.69 (1H, d, J = 8.4 Hz) |
| 142 | 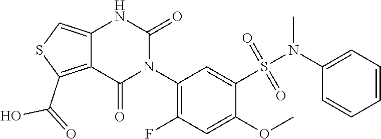 [SMILES: COc1cc(F)c(-n2c(=O)[nH]c3csc(C(=O)O)c3c2=O)cc1S(=O)(=O)N(C)c1ccccc1] | (DMSO-d6) 3.3 (3H, s), 3.87 (3H, s), 7.15-7.4 (6H, m), 7.42 (1H, d, J = 11.7 Hz), 7.92 (1H, d, J = 8.3 Hz), 11.95 (1H, s), 14.39 (1H, s) |
| 143 | 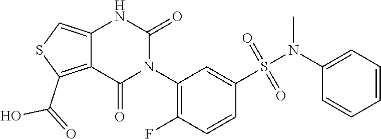 [SMILES: CN(c1ccccc1)S(=O)(=O)c1ccc(F)c(-n2c(=O)[nH]c3csc(C(=O)O)c3c2=O)c1] | (DMSO-d6) 3.17 (3H, s), 7.05-7.15 (2H, m), 7.25-7.4 (4H, m), 7.6-7.75 (2H, m), 7.9-8.0 (1H, m), 12.02 (1H, s), 14.29 (1H, s) |
| 144 | 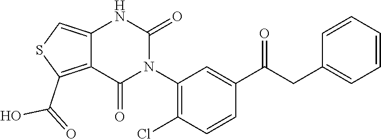 [SMILES: O=C(Cc1ccccc1)c1ccc(Cl)c(-n2c(=O)[nH]c3csc(C(=O)O)c3c2=O)c1] | (DMSO-d6) 4.42 (2H, s), 7.2-7.45 (6H, m), 7.86 (1H, d, J = 8.5 Hz), 8.2 (1H, dd, J = 8.5 Hz, 2.2 Hz), 8.29 (1H, d, J = 2.2 Hz), 12.06 (1H, brs), 14.32 (1H, brs) |
| 145 | 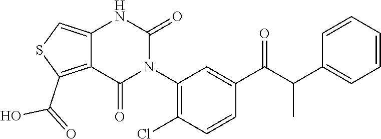 [SMILES: CC(C(=O)c1ccc(Cl)c(-n2c(=O)[nH]c3csc(C(=O)O)c3c2=O)c1)c1ccccc1] | (DMSO-d6) 1.4-1.5 (3H, m), 4.9-5.0 (1H, m), 7.15-7.45 (6H, m), 7.7-7.8 (1H, m), 8.1-8.15 (1H, m), 8.2-8.3 (1H, m), 12.0-12.1 (1H, m), 14.2-14.35 (1H, m) |
| 146 | 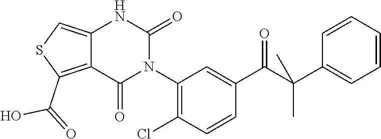 [SMILES: CC(C)(C(=O)c1ccc(Cl)c(-n2c(=O)[nH]c3csc(C(=O)O)c3c2=O)c1)c1ccccc1] | (DMSO-d6) 1.57 (6H, s), 7.25-7.45 (7H, m), 7.5-7.6 (1H, m), 7.9-8.0 (1H, m), 11.9-12.1 (1H, br), 14.2-14.4 (1H, br) |
| 147 | 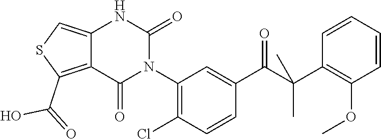 [SMILES: COc1ccccc1C(C)(C)C(=O)c1ccc(Cl)c(-n2c(=O)[nH]c3csc(C(=O)O)c3c2=O)c1] | (DMSO-d6) 1.55 (3H, s), 1.57 (3H, s), 3.33 (3H, s), 6.8-6.85 (1H, m), 7.0-7.1 (1H, m), 7.2-7.3 (1H, m), 7.3-7.5 (3H, m), 7.5-7.55 (1H, m), 7.94 (1H, d, J = 2.4 Hz), 12.0 (1H, s), 14.37 (1H, brs) |
| 148 | 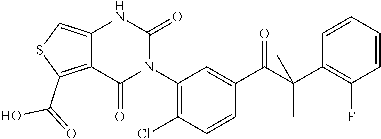 [SMILES: CC(C)(C(=O)c1ccc(Cl)c(-n2c(=O)[nH]c3csc(C(=O)O)c3c2=O)c1)c1ccccc1F] | (DMSO-d6) 1.6 (3H, s), 1.62 (3H, s), 7.0-7.15 (1H, m), 7.25-7.4 (3H, m), 7.47 (1H, dd, J = 8.7 Hz, 2.0 Hz), 7.57 (1H, d, J = 8.7 Hz), 7.65-7.75 (1H, m), 8.01 (1H, d, J = 2.0 Hz), 12.03 (1H, s), 14.3 (1H, brs) |
| 149 | 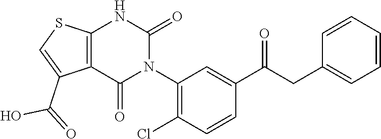 [SMILES: O=C(Cc1ccccc1)c1ccc(Cl)c(-n2c(=O)[nH]c3scc(C(=O)O)c3c2=O)c1] | (DMSO-d6) 4.42 (2H, s), 7.2-7.35 (5H, m), 7.86 (1H, d, J = 8.5 Hz), 7.94 (1H, s), 8.15-8.3 (2H, m), 13.84 (1H, s) |
| 150 | 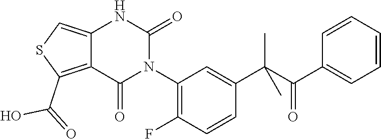 [SMILES: CC(C)(C(=O)c1ccccc1)c1ccc(F)c(-n2c(=O)[nH]c3csc(C(=O)O)c3c2=O)c1] | (DMSO-d6) 1.55 (6H, s), 7.25-7.55 (8H, m), 7.6-7.65 (1H, m), 11.99 (1H, s), 14.46 (1H, brs) |
| 151 | 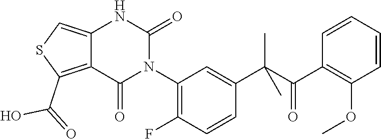 [SMILES: COc1ccccc1C(=O)C(C)(C)c1ccc(F)c(-n2c(=O)[nH]c3csc(C(=O)O)c3c2=O)c1] | (DMSO-d6) 1.49 (3H, s), 1.5 (3H, s), 3.66 (3H, s), 6.55-6.65 (1H, m), 6.75- 6.85 (1H, m), 6.95-7.05 (1H, m), 7.25- 7.5 (4H, m), 7.5-7.6 (1H, m), 11.99 (1H, s), 14.5 (1H, s) |
| 152 | 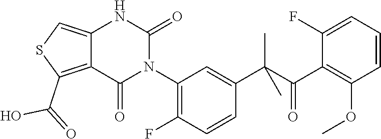 [SMILES: COc1cccc(F)c1C(=O)C(C)(C)c1ccc(F)c(-n2c(=O)[nH]c3csc(C(=O)O)c3c2=O)c1] | (DMSO-d6) 1.536 (3H, s), 1.543 (3H, s), 3.61 (3H, s), 6.75-6.85 (2H, m), 7.3-7.4 (3H, m), 7.45-7.6 (2H, m), 11.98 (1H, s), 14.5 (1H, s) |
| 153 | 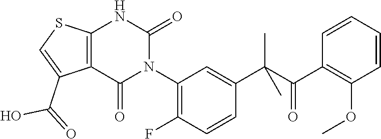 [SMILES: COc1ccccc1C(=O)C(C)(C)c1ccc(F)c(-n2c(=O)[nH]c3scc(C(=O)O)c3c2=O)c1] | (DMSO-d6) 1.49 (3H, s), 1.5 (3H, s), 3.66 (3H, s), 6.55-6.65 (1H, m), 6.75- 6.85 (1H, m), 6.95-7.05 (1H, m), 7.25- 7.55 (4H, m), 7.94 (1H, s), 12.8-13.1 (1H, br), 14.01 (1H, s) |
| 154 | 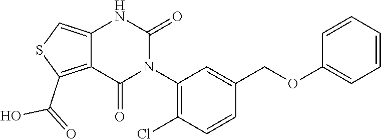 [SMILES: O=C(O)c1scc2[nH]c(=O)n(-c3cc(COc4ccccc4)ccc3Cl)c(=O)c12] | (DMSO-d6) 5.15 (2H, s), 6.9-7.1 (3H, m), 7.25-7.35 (2H, m), 7.4 (1H, s), 7.55- 7.65 (1H, m), 7.65-7.75 (2H, m), 12.05 (1H, s), 14.42 (1H, s) |
| 155 | 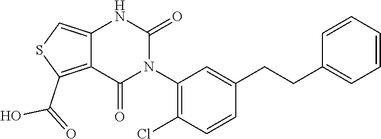 [SMILES: O=C(O)c1scc2[nH]c(=O)n(-c3cc(CCc4ccccc4)ccc3Cl)c(=O)c12] | (DMSO-d6) 2.85-2.95 (4H, m), 7.1-7.6 (9H, m), 12.04 (1H, s), 14.46 (1H, s) |
| 156 | 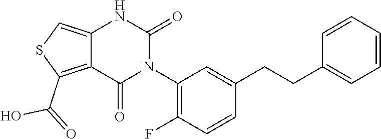 [SMILES: O=C(O)c1scc2[nH]c(=O)n(-c3cc(CCc4ccccc4)ccc3F)c(=O)c12] | (DMSO-d6) 2.8-3.0 (4H, m), 7.15-7.45 (9H, m), 12.03 (1H, s), 14.46 (1H, brs) |
| 157 | 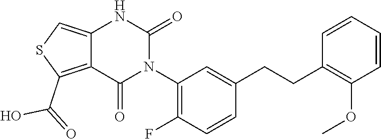 [SMILES: COc1ccccc1CCc1ccc(F)c(-n2c(=O)[nH]c3csc(C(=O)O)c3c2=O)c1] | (DMSO-d6) 2.8-2.9 (4H, m), 3.79 (3H, s), 6.8-6.9 (1H, m), 6.9-7.0 (1H, m), 7.1-7.25 (2H, m), 7.25-7.45 (4H, m), 12.02 (1H, s), 14.48 (1H, brs) |
| 158 | 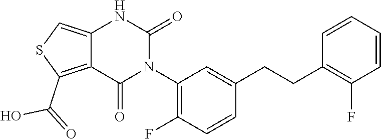 [SMILES: O=C(O)c1scc2[nH]c(=O)n(-c3cc(CCc4ccccc4F)ccc3F)c(=O)c12] | (DMSO-d6) 2.85-3.0 (4H, m), 7.05-7.2 (2H, m), 7.2-7.45 (6H, m), 12.03 (1H, s), 14.46 (1H, brs) |
| 159 | 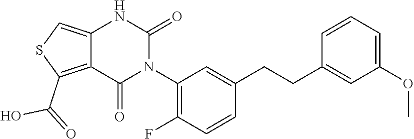 [SMILES: COc1cccc(CCc2ccc(F)c(-n3c(=O)[nH]c4csc(C(=O)O)c4c3=O)c2)c1] | (DMSO-d6) 2.8-2.95 (4H, m), 3.72 (3H, s), 6.7-6.85 (3H, m), 7.15-7.25 (1H, m), 7.25-7.45 (4H, m), 11.95-12.1 (1H, br), 14.35-14.55 (1H, br) |
| 160 | 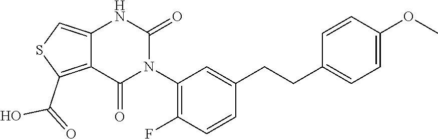 [SMILES: COc1ccc(CCc2ccc(F)c(-n3c(=O)[nH]c4csc(C(=O)O)c4c3=O)c2)cc1] | (DMSO-d6) 2.75-2.95 (4H, m), 3.71 (3H, s), 6.8-6.9 (2H, m), 7.1-7.2 (2H, m), 7.25-7.45 (4H, m), 12.01 (1H, brs), 14.4- 14.55 (1H, br) |
| 161 | 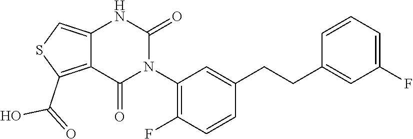 [SMILES: O=C(O)c1scc2[nH]c(=O)n(-c3cc(CCc4cccc(F)c4)ccc3F)c(=O)c12] | (DMSO-d6) 2.85-3.0 (4H, m), 6.95-7.05 (1H, m), 7.05-7.15 (2H, m), 7.25-7.45 (5H, m), 12.03 (1H, brs), 14.3-14.6 (1H, br) |
| 162 | 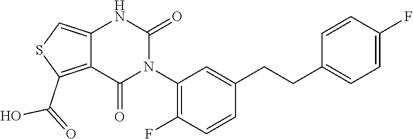 [SMILES: O=C(O)c1scc2[nH]c(=O)n(-c3cc(CCc4ccc(F)cc4)ccc3F)c(=O)c12] | (DMSO-d6) 2.8-2.95 (4H, m), 7.05-7.15 (2H, m), 7.2-7.45 (6H, m), 12.03 (1H, brs), 14.3-14.6 (1H, br) |
| 163 | 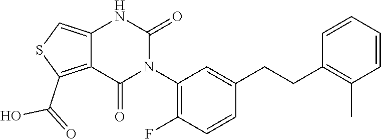 [SMILES: Cc1ccccc1CCc1ccc(F)c(-n2c(=O)[nH]c3csc(C(=O)O)c3c2=O)c1] | (DMSO-d6) 2.28 (3H, s), 2.8-2.9 (4H, m), 7.05-7.25 (4H, m), 7.25-7.5 (4H, m), 12.04 (1H, brs), 14.47 (1H, brs) |
| 164 | 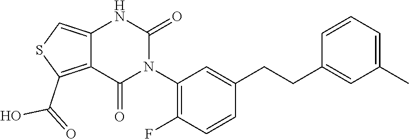 [SMILES: Cc1cccc(CCc2ccc(F)c(-n3c(=O)[nH]c4csc(C(=O)O)c4c3=O)c2)c1] | (DMSO-d6) 2.27 (3H, s), 2.8-2.95 (4H, m), 6.95-7.1 (3H, m), 7.1-7.2 (1H, m), 7.25-7.45 (4H, m), 12.03 (1H, brs), 14.47 (1H, brs) |
| 165 | 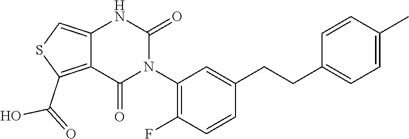 [SMILES: Cc1ccc(CCc2ccc(F)c(-n3c(=O)[nH]c4csc(C(=O)O)c4c3=O)c2)cc1] | (DMSO-d6) 2.25 (3H, s), 2.8-2.95 (4H, m), 7.05-7.15 (4H, m), 7.25-7.45 (4H, m), 12.03 (1H, brs), 14.35-14.6 (1H, br) |
| 166 | 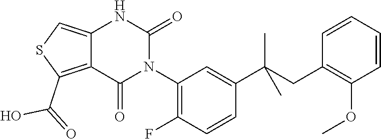 [SMILES: COc1ccccc1CC(C)(C)c1ccc(F)c(-n2c(=O)[nH]c3csc(C(=O)O)c3c2=O)c1] | (DMSO-d6) 1.23 (3H, s), 1.25 (3H, s), 2.85 (2H, s), 3.67 (3H, s), 6.7-6.8 (2H, m), 6.85-6.95 (1H, m), 7.1-7.2 (1H, m), 7.25-7.35 (1H, m), 7.35-7.45 (2H, m), 7.5-7.6 (1H, m), 12.0 (1H, s), 14.55 (1H, s) |
| 167 | 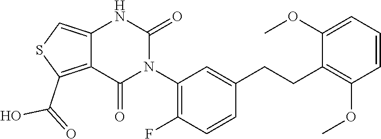 [SMILES: COc1cccc(OC)c1CCc1ccc(F)c(-n2c(=O)[nH]c3csc(C(=O)O)c3c2=O)c1] | (DMSO-d6) 2.65-2.75 (2H, m), 2.8-2.9 (2H, m), 3.75 (6H, s), 6.55-6.7 (2H, m), 7.1-7.2 (1H, m), 7.25-7.45 (4H, m), 12.0 (1H, s), 14.48 (1H, brs) |
| 168 | 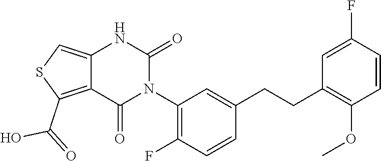 [SMILES: COc1ccc(F)cc1CCc1ccc(F)c(-n2c(=O)[nH]c3csc(C(=O)O)c3c2=O)c1] | (DMSO-d6) 2.8-2.95 (4H, m), 3.77 (3H, s), 6.9-7.1 (3H, m), 7.25-7.45 (4H, m), 12.01 (1H, s), 14.46 (1H, brs) |
| 169 | 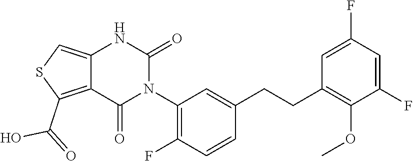 [SMILES: COc1c(F)cc(F)cc1CCc1ccc(F)c(-n2c(=O)[nH]c3csc(C(=O)O)c3c2=O)c1] | (DMSO-d6) 2.85-2.95 (4H, m), 3.75 (3H, s), 6.9-7.05 (1H, m), 7.05-7.2 (1H, m), 7.3-7.45 (4H, m), 12.01 (1H, s), 14.45 (1H, brs) |
| 170 | 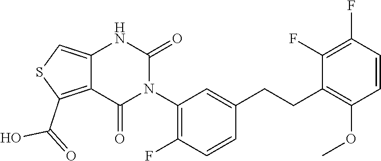 [SMILES: COc1ccc(F)c(F)c1CCc1ccc(F)c(-n2c(=O)[nH]c3csc(C(=O)O)c3c2=O)c1] | (DMSO-d6) 2.75-2.85 (2H, m), 2.85- 2.95 (2H, m), 3.77 (3H, s), 6.75-6.85 (1H, m), 7.15-7.35 (4H, m), 7.37 (1H, s), 11.99 (1H, s), 14.46 (1H, brs) |
| 171 | 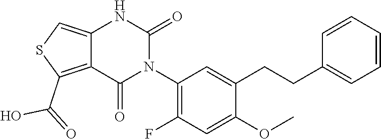 [SMILES: COc1cc(F)c(-n2c(=O)[nH]c3csc(C(=O)O)c3c2=O)cc1CCc1ccccc1] | (DMSO-d6) 2.75-2.9 (4H, m), 3.86 (3H, s), 7.08 (1H, d, J = 12.2 Hz), 7.15-7.35 (6H, m), 7.38 (1H, s), 11.97 (1H, s), 14.55 (1H, brs) |
| 172 | 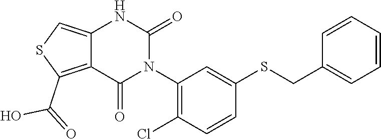 [SMILES: O=C(O)c1scc2[nH]c(=O)n(-c3cc(SCc4ccccc4)ccc3Cl)c(=O)c12] | (DMSO-d6) 4.28 (2H, s), 7.2-7.35 (3H, m), 7.35-7.5 (4H, m), 7.58 (1H, d, J = 8.5 Hz), 7.66 (1H, d, J = 2.2 Hz), 12.06 (1H, s), 14.41 (1H, s) |
| 173 | 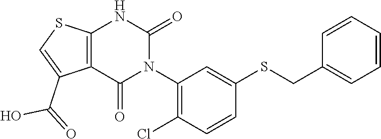 [SMILES: O=C(O)c1csc2[nH]c(=O)n(-c3cc(SCc4ccccc4)ccc3Cl)c(=O)c12] | (DMSO-d6) 4.29 (2H, s), 7.2-7.45 (6H, m), 7.57 (1H, d, J = 8.5 Hz), 7.64 (1H, d, J = 2.3 Hz), 7.94 (1H, s), 13.03 (1H, s), 13.94 (1H, s) |
| 174 | 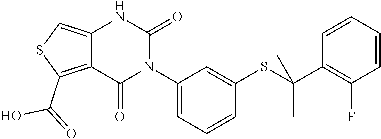 [SMILES: CC(C)(Sc1cccc(-n2c(=O)[nH]c3csc(C(=O)O)c3c2=O)c1)c1ccccc1F] | (DMSO-d6) 1.69 (6H, s), 7.0-7.2 (4H, m), 7.25-7.4 (5H, m), 11.89 (1H, s), 14.86 (1H, s) |
| 175 | 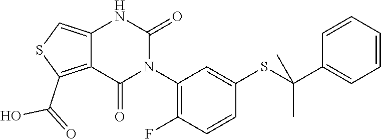 [SMILES: CC(C)(Sc1ccc(F)c(-n2c(=O)[nH]c3csc(C(=O)O)c3c2=O)c1)c1ccccc1] | (DMSO-d6) 4.24 (2H, s), 7.2-7.5 (8H, m), 7.62 (1H, dd, J = 6.7 Hz, 2.2 Hz), 12.05 (1H, s), 14.41 (1H, s) |
| 176 | 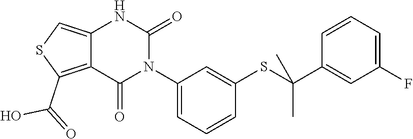 [SMILES: CC(C)(Sc1cccc(-n2c(=O)[nH]c3csc(C(=O)O)c3c2=O)c1)c1cccc(F)c1] | (DMSO-d6) 1.65 (6H, s), 7.0-7.1 (1H, m), 7.1-7.45 (8H, m), 11.88 (1H, s), 14.83 (1H, s) |
| 177 | 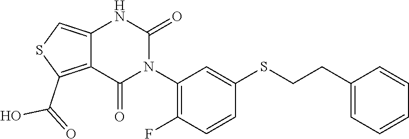 [SMILES: O=C(O)c1scc2[nH]c(=O)n(-c3cc(SCCc4ccccc4)ccc3F)c(=O)c12] | (DMSO-d6) 2.8-2.9 (2H, m), 3.15-3.25 (2H, m), 7.15-7.55 (8H, m), 7.58 (1H, dd, J = 6.8 Hz, 2.3 Hz) |
| 178 | 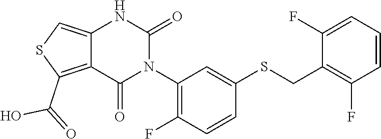 [SMILES: O=C(O)c1scc2[nH]c(=O)n(-c3cc(SCc4c(F)cccc4F)ccc3F)c(=O)c12] | (DMSO-d6) 4.21 (2H, s), 7.0-7.15 (2H, m), 7.3-7.45 (3H, m), 7.5-7.65 (2H, m), 12.0 (1H, s), 14.37 (1H, s) |
| 179 | 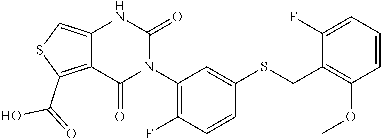 [SMILES: COc1cccc(F)c1CSc1ccc(F)c(-n2c(=O)[nH]c3csc(C(=O)O)c3c2=O)c1] | (DMSO-d6) 3.76 (3H, s), 4.15 (2H, s), 6.75-6.9 (2H, m), 7.25-7.35 (2H, m), 7.35-7.45 (1H, m), 7.45-7.6 (2H, m) |
| 180 | 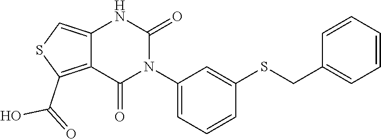 [SMILES: O=C(O)c1scc2[nH]c(=O)n(-c3cccc(SCc4ccccc4)c3)c(=O)c12] | (DMSO-d6) 4.27 (2H, s), 7.15-7.5 (10H, m), 11.93 (1H, s), 14.88 (1H, s) |
| 181 | 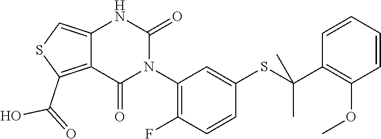 [SMILES: COc1ccccc1C(C)(C)Sc1ccc(F)c(-n2c(=O)[nH]c3csc(C(=O)O)c3c2=O)c1] | (DMSO-d6) 1.65-1.7 (6H, m), 3.86 (3H, s) 6.75-6.85 (1H, m), 7.0-7.15 (3H, m), 7.2-7.3 (2H, m), 7.37 (1H, s), 7.42 (1H, dd, J = 6.9 Hz, 2.2 Hz), 11.98 (1H, s), 14.38 (1H, s) |
| 182 | 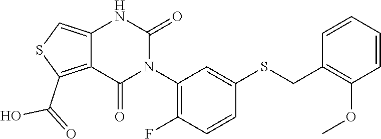 [SMILES: COc1ccccc1CSc1ccc(F)c(-n2c(=O)[nH]c3csc(C(=O)O)c3c2=O)c1] | (DMSO-d6) 3.79 (3H, s), 4.16 (2H, s), 6.8-6.9 (1H, m), 6.95-7.05 (1H, m), 7.2- 7.3 (2H, m), 7.3-7.5 (3H, m), 7.59 (1H, dd, J = 7.0 hz, 2.2 Hz), 12.02 (1H, s), 14.39 (1H, s) |
| 183 | 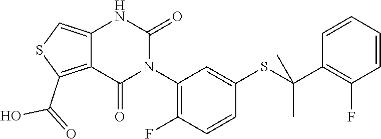 [SMILES: CC(C)(Sc1ccc(F)c(-n2c(=O)[nH]c3csc(C(=O)O)c3c2=O)c1)c1ccccc1F] | (DMSO-d6) 1.69 (6H, s), 7.0-7.35 (6H, m), 7.37 (1H, s), 7.46 (1H, dd, J = 6.7 Hz, 2.2 Hz), 11.98 (1H, s), 14.37 (1H, brs) |
| 184 | 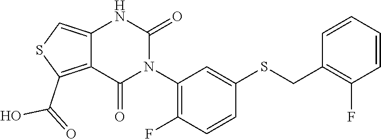 [SMILES: O=C(O)c1scc2[nH]c(=O)n(-c3cc(SCc4ccccc4F)ccc3F)c(=O)c12] | (DMSO-d6) 4.24 (2H, s), 7.05-7.25 (2H, m), 7.25-7.45 (4H, m), 7.45-7.55 (1H, m), 7.63 (1H, dd, J = 6.8 Hz, 2.5 Hz), 12.03 (1H, s), 14.38 (1H, s) |
| 185 | 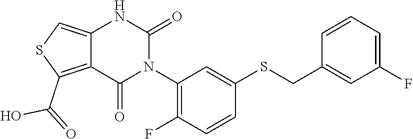 [SMILES: O=C(O)c1scc2[nH]c(=O)n(-c3cc(SCc4cccc(F)c4)ccc3F)c(=O)c12] | (DMSO-d6) 4.25 (2H, s), 7.0-7.1 (1H, m), 7.15-7.2 (2H, m), 7.25-7.5 (4H, m), 7.62 (1H, dd, J = 6.7 Hz, 2.3 Hz), 12.03 (1H, s), 14.37 (1H, s) |
| 186 | 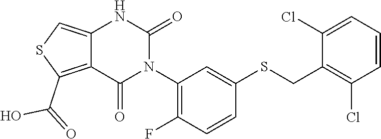 [SMILES: O=C(O)c1scc2[nH]c(=O)n(-c3cc(SCc4c(Cl)cccc4Cl)ccc3F)c(=O)c12] | (DMSO-d6) 4.4 (2H, s), 7.3-7.6 (6H, m), 7.65-7.75 (1H, m), 12.01 (1H, s), 14.37 (1H, s) |
| 187 | 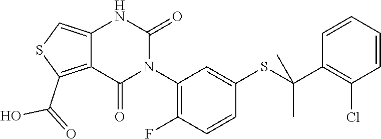 [SMILES: CC(C)(Sc1ccc(F)c(-n2c(=O)[nH]c3csc(C(=O)O)c3c2=O)c1)c1ccccc1Cl] | (DMSO-d6) 1.75-1.8 (6H, m), 7.05-7.15 (1H, m), 7.15-7.3 (4H, m), 7.37 (1H, s), 7.4-7.5 (2H, m), 11.97 (1H, s), 14.37 (1H, s) |
| 188 | 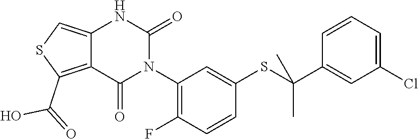 [SMILES: CC(C)(Sc1ccc(F)c(-n2c(=O)[nH]c3csc(C(=O)O)c3c2=O)c1)c1cccc(Cl)c1] | (DMSO-d6) 1.65 (6H, s), 7.15-7.25 (1H, m), 7.25-7.45 (6H, m), 7.5-7.6 (1H, m), 12.0 (1H, s), 14.38 (1H, s) |
| 189 | 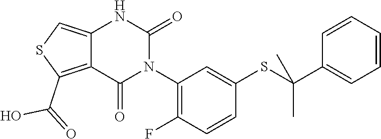 [SMILES: CC(C)(Sc1ccc(F)c(-n2c(=O)[nH]c3csc(C(=O)O)c3c2=O)c1)c1ccccc1] | (DMSO-d6) 1.64 (6H, s), 7.15-7.35 (5H, m), 7.37 (1H, s), 7.4-7.45 (2H, m), 7.52 (1H, dd, J = 7.3 Hz, 2.3 Hz), 11.99 (1H, s), 14.37 (1H, brs) |
| 190 | 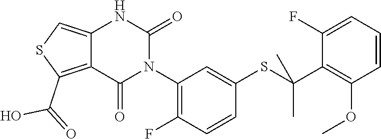 [SMILES: COc1cccc(F)c1C(C)(C)Sc1ccc(F)c(-n2c(=O)[nH]c3csc(C(=O)O)c3c2=O)c1] | (DMSO-d6) 1.75-1.85 (6H, m), 3.77 (3H, s), 6.6-6.7 (1H, m), 6.85 (1H, d, J = 8.1 Hz), 7.05-7.15 (1H, m), 7.2-7.35 (2H, m), 7.37 (1H, s), 7.4-7.45 (1H, m), 11.97 (1H, s), 14.4 (1H, brs) |
| 191 | 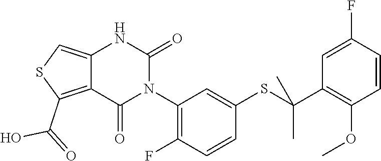 [SMILES: COc1ccc(F)cc1C(C)(C)Sc1ccc(F)c(-n2c(=O)[nH]c3csc(C(=O)O)c3c2=O)c1] | (DMSO-d6) 1.65-1.7 (6H, m), 3.85 (3H, s), 6.75-6.8 (1H, m), 7.0-7.1 (2H, m), 7.1-7.2 (1H, m), 7.25-7.35 (1H, m), 7.37 (1H, s), 7.4 (1H, dd, J = 7.3 Hz, 2.2 hz), 11.98 (1H, s), 14.39 (1H, brs) |
| 192 | 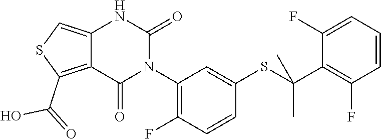 [SMILES: CC(C)(Sc1ccc(F)c(-n2c(=O)[nH]c3csc(C(=O)O)c3c2=O)c1)c1c(F)cccc1F] | (DMSO-d6) 1.81 (6H, s), 6.9-7.05 (2H, m), 7.1-7.2 (1H, m), 7.25-7.4 (3H, m), 7.45-7.5 (1H, m), 11.97 (1H, s), 14.38 (1H, brs) |
| 193 | 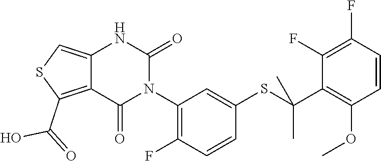 [SMILES: COc1ccc(F)c(F)c1C(C)(C)Sc1ccc(F)c(-n2c(=O)[nH]c3csc(C(=O)O)c3c2=O)c1] | (DMSO-d6) 1.8-1.85 (6H, m), 3.76 (3H, s), 6.75-6.85 (1H, m), 7.15-7.45 (5H, m), 11.98 (1H, s), 14.38 (1H, brs) |
| 194 | 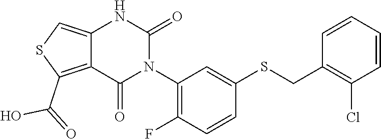 [SMILES: O=C(O)c1scc2[nH]c(=O)n(-c3cc(SCc4ccccc4Cl)ccc3F)c(=O)c12] | (DMSO-d6) 4.29 (2H, s), 7.2-7.35 (2H, m), 7.35-7.55 (5H, m), 7.6-7.7 (1H, m), 12.02 (1H, s), 14.38 (1H, brs) |
| 195 | 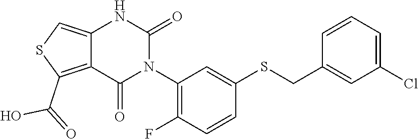 [SMILES: O=C(O)c1scc2[nH]c(=O)n(-c3cc(SCc4cccc(Cl)c4)ccc3F)c(=O)c12] | (DMSO-d6) 4.24 (2H, s), 7.25-7.5 (7H, m), 7.6-7.65 (1H, m), 12.03 (1H, s), 14.38 (1H, brs) |
| 196 | 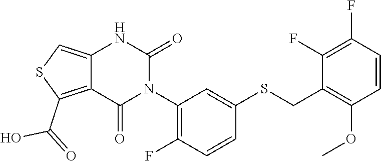 [SMILES: COc1ccc(F)c(F)c1CSc1ccc(F)c(-n2c(=O)[nH]c3csc(C(=O)O)c3c2=O)c1] | (DMSO-d6) 3.73 (3H, s), 4.16 (2H, s), 6.75-6.85 (1H, m), 7.1-7.6 (5H, m) |
| 197 | 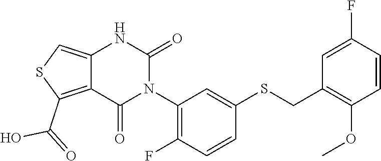 [SMILES: COc1ccc(F)cc1CSc1ccc(F)c(-n2c(=O)[nH]c3csc(C(=O)O)c3c2=O)c1] | (DMSO-d6) 3.77 (3H, s), 4.15 (2H, s), 6.95-7.15 (3H, m), 7.35-7.5 (3H, m), 7.6-7.65 (1H, m), 12.06 (1H, s), 14.41 (1H, s) |
| 198 | 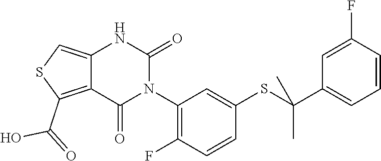 [SMILES: CC(C)(Sc1ccc(F)c(-n2c(=O)[nH]c3csc(C(=O)O)c3c2=O)c1)c1cccc(F)c1] | (DMSO-d6) 1.6-1.7 (6H, m), 7.0-7.1 (1H, m), 7.15-7.25 (3H, m), 7.3-7.4 (3H, m), 7.5-7.55 (1H, m), 12.01 (1H, s), 14.4 (1H, s) |
| 199 | 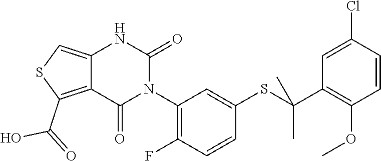 [SMILES: COc1ccc(Cl)cc1C(C)(C)Sc1ccc(F)c(-n2c(=O)[nH]c3csc(C(=O)O)c3c2=O)c1] | (DMSO-d6) 1.66 (3H, s), 1.67 (3H, s), 3.86 (3H, s), 7.0 (1H, d, J = 2.5 Hz), 7.06 (1H, d, J = 8.8 Hz), 7.1-7.2 (1H, m), 7.25-7.35 (2H, m), 7.38 (1H, s), 7.4-7.5 (1H, m), 12.01 (1H, s), 14.41 (1H, brs) |
| 200 | 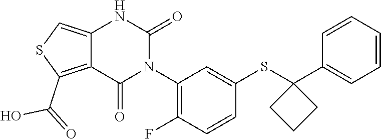 [SMILES: O=C(O)c1scc2[nH]c(=O)n(-c3cc(SC4(c5ccccc5)CCC4)ccc3F)c(=O)c12] | (DMSO-d6) 1.75-1.9 (1H, m), 2.15-2.3 (1H, m), 2.4-2.65 (4H, m), 7.0-7.2 (4H, m), 7.2-7.35 (3H, m), 7.37 (1H, s), 7.45- 7.5 (1H, m), 11.98 (1H, s), 14.41 (1H, brs) |
| 201 | 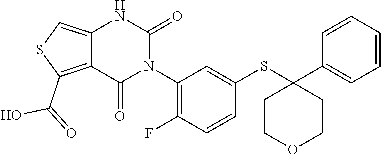 [SMILES: O=C(O)c1scc2[nH]c(=O)n(-c3cc(SC4(c5ccccc5)CCOCC4)ccc3F)c(=O)c12] | (DMSO-d6) 2.05-2.25 (4H, m), 3.5-3.65 (2H, m), 3.85-3.95 (2H, m), 6.85-6.95 (1H, m), 7.15-7.4 (8H, m), 11.98 (1H, s), 14.38 (1H, brs) |
| 202 | 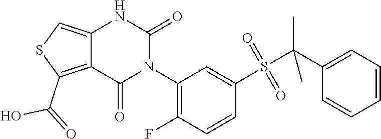 [SMILES: CC(C)(c1ccccc1)S(=O)(=O)c1ccc(F)c(-n2c(=O)[nH]c3csc(C(=O)O)c3c2=O)c1] | (DMSO-d6) 1.73 (6H, s), 7.3-7.4 (7H, m), 7.5-7.6 (1H, m), 7.95 (1H, dd, J = 6.7 Hz, 2.2 Hz), 12.01 (1H, s), 14.25 (1H, s) |
| 203 | 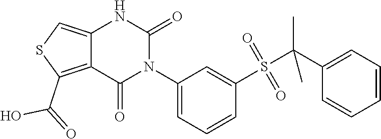 [SMILES: CC(C)(c1ccccc1)S(=O)(=O)c1cccc(-n2c(=O)[nH]c3csc(C(=O)O)c3c2=O)c1] | (DMSO-d6) 1.7 (6H, s), 6.89 (1H, s), 7.2-7.4 (6H, m), 7.5-7.7 (3H, m) |
| 204 | 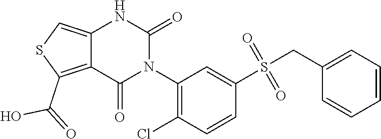 [SMILES: O=C(O)c1scc2[nH]c(=O)n(-c3cc(S(=O)(=O)Cc4ccccc4)ccc3Cl)c(=O)c12] | (DMSO-d6) 4.77 (2H, s), 7.15-7.25 (2H, m), 7.25-7.35 (3H, m), 7.39 (1H, s), 7.87 (1H, dd, J = 8.6 Hz, 2.2 Hz), 7.95 (1H, d, J = 8.6 Hz), 8.09 (1H, d, J = 2.2 Hz), 12.08 (1H, s), 14.26 (1H, s) |
| 205 | 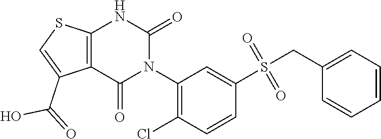 [SMILES: O=C(O)c1csc2[nH]c(=O)n(-c3cc(S(=O)(=O)Cc4ccccc4)ccc3Cl)c(=O)c12] | (DMSO-d6) 4.77 (2H, s), 7.15-7.25 (2H, m), 7.25-7.35 (3H, m), 7.8-8.0 (3H, m), 8.08 (1H, d, J = 2.3 Hz), 13.78 (1H, s) |
| 206 | 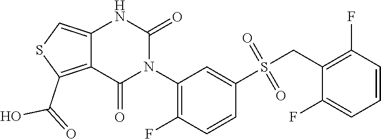 [SMILES: O=C(O)c1scc2[nH]c(=O)n(-c3cc(S(=O)(=O)Cc4c(F)cccc4F)ccc3F)c(=O)c12] | (DMSO-d6) 4.76 (2H, s), 7.05-7.15 (2H, m), 7.36 (1H, s), 7.4-7.55 (1H, m), 7.7- 7.8 (1H, m), 7.95-8.1 (2H, m), 12.02 (1H, s), 14.26 (1H, s) |
| 207 | 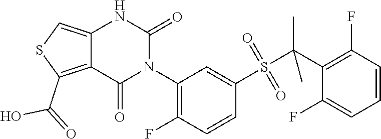 [SMILES: CC(C)(c1c(F)cccc1F)S(=O)(=O)c1ccc(F)c(-n2c(=O)[nH]c3csc(C(=O)O)c3c2=O)c1] | (DMSO-d6) 1.89 (6H, s), 7.0-7.1 (2H, m), 7.36 (1H, s), 7.4-7.5 (1H, m), 7.6-7.7 (2H, m), 8.0-8.01 (1H, m), 12.0 (1H, s), 14.28 (1H, s) |
| 208 | 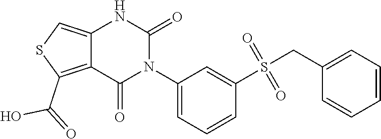 [SMILES: O=C(O)c1scc2[nH]c(=O)n(-c3cccc(S(=O)(=O)Cc4ccccc4)c3)c(=O)c12] | (DMSO-d6) 4.72 (2H, s), 7.15-7.25 (2H, m), 7.25-7.35 (3H, m), 7.39 (1H, s), 7.7- 7.95 (4H, m), 11.97 (1H, s), 14.72 (1H, s) |
| 209 | 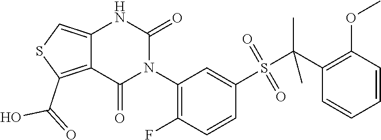 [SMILES: COc1ccccc1C(C)(C)S(=O)(=O)c1ccc(F)c(-n2c(=O)[nH]c3csc(C(=O)O)c3c2=O)c1] | (DMSO-d6) 1.86 (6H, s), 3.35 (3H, s), 6.83 (1H, d, J = 8.6 Hz), 6.9-7.0 (1H, m), 7.25-7.35 (2H, m), 7.37 (1H, s), 7.4-7.45 (1H, m), 7.5-7.6 (1H, m), 7.85-7.96 (1H, m), 12.0 (1H, s), 14.29 (1H, s) |
| 210 | 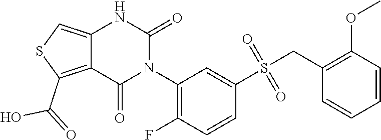 [SMILES: COc1ccccc1CS(=O)(=O)c1ccc(F)c(-n2c(=O)[nH]c3csc(C(=O)O)c3c2=O)c1] | (DMSO-d6) 3.46 (3H, s), 4.63 (2H, s), 6.85-6.95 (2H, m), 7.2-7.35 (2H, m), 7.37 (1H, s), 7.6-7.75 (2H, m), 7.9-8.0 (1H, m), 12.02 (1H, s), 14.26 (1H, s) |
| 211 | 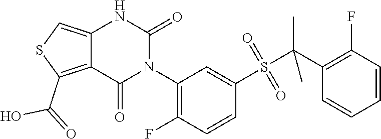 [SMILES: CC(C)(c1ccccc1F)S(=O)(=O)c1ccc(F)c(-n2c(=O)[nH]c3csc(C(=O)O)c3c2=O)c1] | (DMSO-d6) 1.81 (6H, s), 7.05-7.2 (2H, m), 7.3-7.45 (3H, m), 7.5-7.65 (2H, m), 7.98 (1H, dd, J = 6.7 Hz, 2.3 Hz), 12.01 (1H, s), 14.27 (1H, s) |
| 212 | 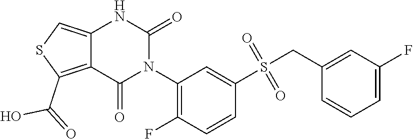 [SMILES: O=C(O)c1scc2[nH]c(=O)n(-c3cc(S(=O)(=O)Cc4cccc(F)c4)ccc3F)c(=O)c12] | (DMSO-d6) 4.81 (2H, s), 6.95-7.05 (2H, m), 7.1-7.2 (1H, m), 7.3-7.4 (2H, m), 7.7-7.75 (1H, m), 7.9-8.0 (1H, m), 8.04 (1H, dd, J = 6.8 Hz, 2.6 Hz), 12.05 (1H, s), 14.24 (1H, s) |
| 213 | 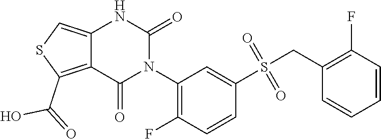 [SMILES: O=C(O)c1scc2[nH]c(=O)n(-c3cc(S(=O)(=O)Cc4ccccc4F)ccc3F)c(=O)c12] | (DMSO-d6) 4.7-4.8 (2H, m), 7.1-7.3 (3H, m), 7.35-7.45 (2H, m), 7.65-7.75 (1H, m), 7.9-8.0 (1H, m), 8.05 (1H, dd, J = 6.8 Hz, 2.5 Hz), 12.03 (1H, s), 14.25 (1H, s) |
| 214 | 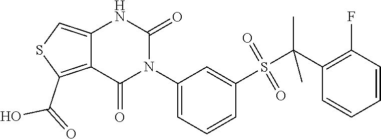 [SMILES: CC(C)(c1ccccc1F)S(=O)(=O)c1cccc(-n2c(=O)[nH]c3csc(C(=O)O)c3c2=O)c1] | (DMSO-d6) 1.75-1.85 (6H, m), 7.05-7.2 (2H, m), 7.3-7.5 (4H, m), 7.6-7.8 (3H, m), 11.91 (1H, s), 14.72 (1H, s) |
| 215 | 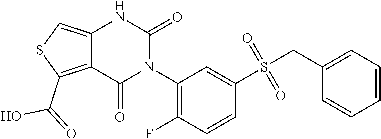 [SMILES: O=C(O)c1scc2[nH]c(=O)n(-c3cc(S(=O)(=O)Cc4ccccc4)ccc3F)c(=O)c12] | (DMSO-d6) 4.74 (2H, s), 7.15-7.2 (2H, m), 7.25-7.35 (3H, m), 7.37 (1H, s), 7.65-7.75 (1H, m), 7.85-7.95 (1H, m), 8.07 (1H, dd, J = 6.6 Hz, 2.5 Hz), 12.04 (1H, s), 14.24 (1H, s) |
| 216 | 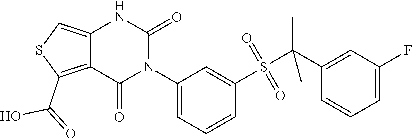 [SMILES: CC(C)(c1cccc(F)c1)S(=O)(=O)c1cccc(-n2c(=O)[nH]c3csc(C(=O)O)c3c2=O)c1] | (DMSO-d6) 1.72 (6H, s), 7.1-7.25 (3H, m), 7.3-7.45 (3H, m), 7.6-7.7 (1H, m), 7.7-7.8 (2H, m), 11.92 (1H, s), 14.7 (1H, s) |
| 217 | 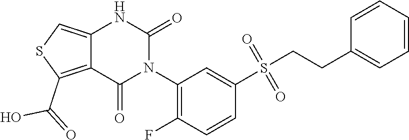 [SMILES: O=C(O)c1scc2[nH]c(=O)n(-c3cc(S(=O)(=O)CCc4ccccc4)ccc3F)c(=O)c12] | (DMSO-d6) 2.85-2.95 (2H, m), 3.65- 3.75 (2H, m), 7.15-7.3 (5H, m), 7.37 (1H, s), 7.7-7.8 (1H, m), 8.1-8.15 (1H, m), 8.27 (1H, dd, J = 6.6 Hz, 2.6 Hz), 12.05 (1H, s), 14.23 (1H, brs) |
| 218 | 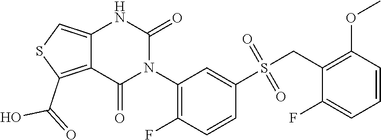 [SMILES: COc1cccc(F)c1CS(=O)(=O)c1ccc(F)c(-n2c(=O)[nH]c3csc(C(=O)O)c3c2=O)c1] | (DMSO-d6) 3.47 (3H, s), 4.62 (2H, s), 6.75 (1H, d, J = 8.4 Hz), 6.8-6.9 (1H, m), 7.3-7.4 (2H, m), 7.65-7.75 (1H, m), 7.8- 7.9 (1H, m), 7.95-8.05 (1H, m), 12.0 (1H, s), 14.27 (1H, brs) |
| 219 | 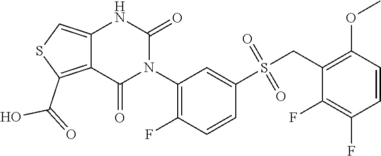 [SMILES: COc1ccc(F)c(F)c1CS(=O)(=O)c1ccc(F)c(-n2c(=O)[nH]c3csc(C(=O)O)c3c2=O)c1] | (DMSO-d6) 3.45 (3H, s), 4.68 (2H, s), 6.7-6.75 (1H, m), 7.35-7.45 (2H, m), 7.65-7.75 (1H, m), 7.85-8.0 (2H, m), 12.01 (1H, s), 14.25 (1H, brs) |
| 220 | 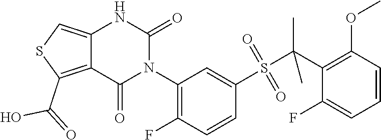 [SMILES: COc1cccc(F)c1C(C)(C)S(=O)(=O)c1ccc(F)c(-n2c(=O)[nH]c3csc(C(=O)O)c3c2=O)c1] | (DMSO-d6) 1.9-2.0 (6H, m), 3.38 (3H, s), 6.65-6.8 (2H, m), 7.3-7.4 (2H, m), 7.4-7.5 (1H, m), 7.55-7.6 (1H, m), 7.95- 8.0 (1H, m), 12.0 (1H, s), 14.28 (1H, brs) |
| 221 | 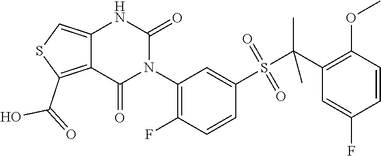 [SMILES: COc1ccc(F)cc1C(C)(C)S(=O)(=O)c1ccc(F)c(-n2c(=O)[nH]c3csc(C(=O)O)c3c2=O)c1] | (DMSO-d6) 1.85 (6H, s), 3.34 (3H, s), 6.84 (1H, dd, J = 9.1 Hz, 5.2 Hz), 7.1- 7.25 (2H, m), 7.37 (1H, s), 7.4-7.45 (1H, m), 7.55-7.65 (1H, m), 7.9-8.0 (1H, m), 12.0 (1H, s), 14.29 (1H, brs) |
| 222 | 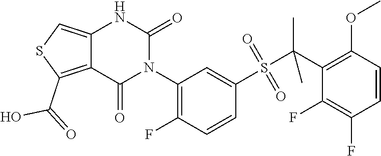 [SMILES: COc1ccc(F)c(F)c1C(C)(C)S(=O)(=O)c1ccc(F)c(-n2c(=O)[nH]c3csc(C(=O)O)c3c2=O)c1] | (DMSO-d6) 1.9-2.0 (6H, m), 3.36 (3H, s), 6.65-6.67 (1H, m), 7.35-7.45 (2H, m), 7.5-7.65 (2H, m), 7.95-8.0 (1H, m), 12.0 (1H, s), 14.28 (1H, brs) |
| 223 | 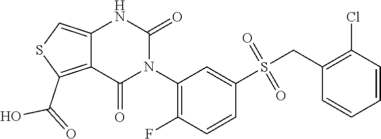 [SMILES: O=C(O)c1scc2[nH]c(=O)n(-c3cc(S(=O)(=O)Cc4ccccc4Cl)ccc3F)c(=O)c12] | (DMSO-d6) 4.85 (2H, s), 7.3-7.45 (5H, m), 7.65-7.75 (1H, m), 7.8-7.9 (1H, m), 8.05-8.1 (1H, m), 12.03 (1H, s), 14.25 (1H, brs) |
| 224 | 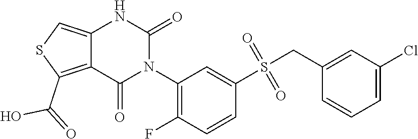 [SMILES: O=C(O)c1scc2[nH]c(=O)n(-c3cc(S(=O)(=O)Cc4cccc(Cl)c4)ccc3F)c(=O)c12] | (DMSO-d6) 4.81 (2H, s), 7.05-7.15 (1H, m), 7.25-7.45 (4H, m), 7.7-7.8 (1H, m), 7.9-8.0 (1H, m), 8.05-8.1 (1H, m), 12.05 (1H, s), 14.24 (1H, brs) |
| 225 | 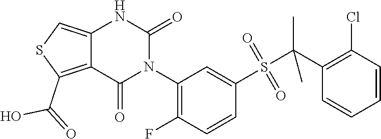 [SMILES: CC(C)(c1ccccc1Cl)S(=O)(=O)c1ccc(F)c(-n2c(=O)[nH]c3csc(C(=O)O)c3c2=O)c1] | (DMSO-d6) 1.95 (6H, s), 7.25-7.45 (5H, m), 7.5-7.65 (2H, m), 8.03 (1H, dd, J = 6.8 Hz, 2.4 Hz), 12.0 (1H, s), 14.28 (1H, s) |
| 226 | 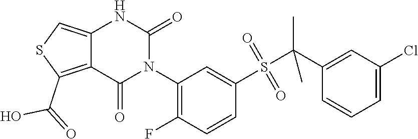 [SMILES: CC(C)(c1cccc(Cl)c1)S(=O)(=O)c1ccc(F)c(-n2c(=O)[nH]c3csc(C(=O)O)c3c2=O)c1] | (DMSO-d6) 1.72 (6H, s), 7.3-7.5 (6H, m), 7.55-7.65 (1H, m), 8.01 (1H, dd, J = 6.7 Hz, 2.3 Hz), 12.02 (1H, s), 14.25 (1H, s) |
| 227 | 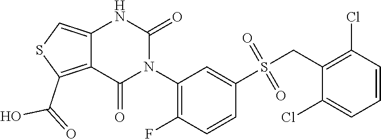 [SMILES: O=C(O)c1scc2[nH]c(=O)n(-c3cc(S(=O)(=O)Cc4c(Cl)cccc4Cl)ccc3F)c(=O)c12] | (DMSO-d6) 4.96 (2H, s), 7.35-7.45 (2H, m), 7.45-7.55 (2H, m), 7.65-7.55 (1H, m), 7.85-7.95 (1H, m), 8.15-8.2 (1H, m), 12.05 (1H, s), 14.28 (1H, s) |
| 228 | 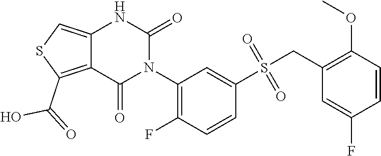 [SMILES: COc1ccc(F)cc1CS(=O)(=O)c1ccc(F)c(-n2c(=O)[nH]c3csc(C(=O)O)c3c2=O)c1] | (DMSO-d6) 3.43 (3H, s), 4.6-4.7 (2H, m), 6.85-6.9 (1H, m), 7.1-7.2 (2H, m), 7.37 (1H, s), 7.65-7.7 (1H, m), 7.75-7.85 (1H, m), 7.9-8.0 (1H, m), 12.04 (1H, s), 14.29 (1H, s) |
| 229 | 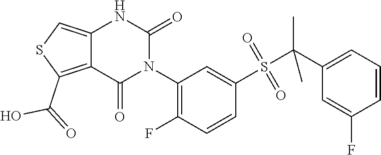 [SMILES: CC(C)(c1cccc(F)c1)S(=O)(=O)c1ccc(F)c(-n2c(=O)[nH]c3csc(C(=O)O)c3c2=O)c1] | (DMSO-d6) 1.72 (3H, s), 1.73 (3H, s), 7.15-7.25 (3H, m), 7.35-7.45 (2H, m), 7.45-7.55 (1H, m), 7.6 (1H, t, J = 9.1 Hz), 7.9-8.0 (1H, m), 12.04 (1H, s), 14.28 (1H, brs) |
| 230 | 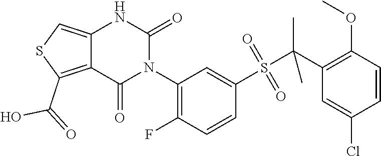 [SMILES: COc1ccc(Cl)cc1C(C)(C)S(=O)(=O)c1ccc(F)c(-n2c(=O)[nH]c3csc(C(=O)O)c3c2=O)c1] | (DMSO-d6) 1.86 (6H, s), 6.87 (1H, d, J = 8.8 Hz), 7.35-7.45 (4H, m), 7.55-7.65 (1H, m), 7.95-8.0 (1H, m), 12.03 (1H, s), 14.3 (1H, brs) |
| 231 | 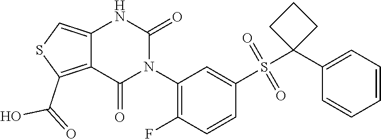 [SMILES: O=C(O)c1scc2[nH]c(=O)n(-c3cc(S(=O)(=O)C4(c5ccccc5)CCC4)ccc3F)c(=O)c12] | (DMSO-d6) 1.8-1.95 (1H, m), 2.0-2.15 (1H, m), 2.55-2.7 (2H, m), 3.0-3.15 (2H, m), 6.95-7.05 (2H, m), 7.2-7.35 (4H, m), 7.37 (1H, s), 7.45-7.55 (1H, m), 7.9- 8.0 (1H, m), 12.0 (1H, s), 14.28 (1H, brs) |
| 232 | 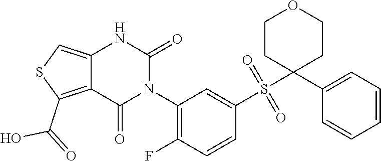 [SMILES: O=C(O)c1scc2[nH]c(=O)n(-c3cc(S(=O)(=O)C4(c5ccccc5)CCOCC4)ccc3F)c(=O)c12] | (DMSO-d6) 2.2-2.35 (2H, m), 3.05-3.2 (2H, m), 3.8-3.9 (2H, m), 7.2-7.4 (7H, m), 7.45-7.55 (1H, m), 7.8-7.9 (1H, m), 12.02 (1H, s), 14.25 (1H, brs) |
| 233 | 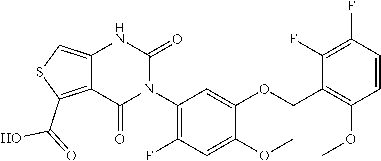 [SMILES: COc1cc(F)c(-n2c(=O)[nH]c3csc(C(=O)O)c3c2=O)cc1OCc1c(OC)ccc(F)c1F] | (DMSO-d6) 3.75-3.85 (6H, m), 4.96 (2H, s), 6.85-6.95 (1H, m), 7.13 (1H, d, J = 11.3 Hz), 7.26 (1H, d, J = 7.2 Hz), 7.39 (1H, s), 7.4-7.55 (1H, m), 12.0 (1H, s), 14.53 (1H, s) |
| 234 | 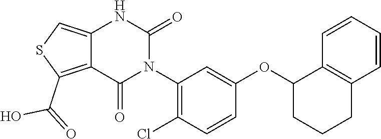 [SMILES: O=C(O)c1scc2[nH]c(=O)n(-c3cc(OC4CCCc5ccccc54)ccc3Cl)c(=O)c12] | (DMSO-d6) 1.7-2.1 (4H, m), 2.65-2.9 (2H, m), 5.45-5.5 (1H, m), 7.1-7.45 (7H, m), 7.57 (1H, d, J = 9.0 Hz), 12.0-12.1 (1H, m), 14.45 (1H, s) |
| 235 | 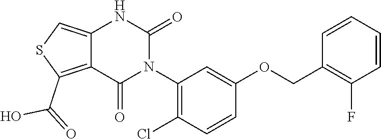 [SMILES: O=C(O)c1scc2[nH]c(=O)n(-c3cc(OCc4ccccc4F)ccc3Cl)c(=O)c12] | (DMSO-d6) 5.14 (2H, s), 7.15-7.5 (6H, m), 7.55-7.65 (2H, m), 12.05 (1H, s), 14.43 (1H, s) |
| 236 | 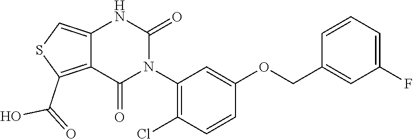 [SMILES: O=C(O)c1scc2[nH]c(=O)n(-c3cc(OCc4cccc(F)c4)ccc3Cl)c(=O)c12] | (DMSO-d6) 5.14 (2H, s), 7.15-7.25 (2H, m), 7.25-7.5 (5H, m), 7.58 (1H, d, J = 9.1 Hz), 12.05 (1H, s), 14.43 (1H, s) |
| 237 | 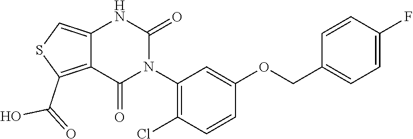 [SMILES: O=C(O)c1scc2[nH]c(=O)n(-c3cc(OCc4ccc(F)cc4)ccc3Cl)c(=O)c12] | (DMSO-d6) 5.09 (2H, s), 7.15-7.3 (3H, m), 7.32 (1H, d, J = 2.9 Hz), 7.41 (1H, s), 7.5-7.6 (3H, m), 12.04 (1H, s), 14.44 (1H, s) |
| 238 | 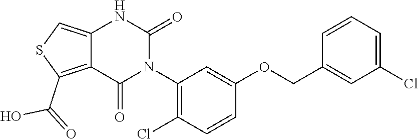 [SMILES: O=C(O)c1scc2[nH]c(=O)n(-c3cc(OCc4cccc(Cl)c4)ccc3Cl)c(=O)c12] | (DMSO-d6) 5.13 (2H, s), 7.19 (1H, dd, J = 9.0 Hz, 3.0 Hz), 7.3-7.5 (5H, m), 7.5- 7.6 (2H, m), 12.05 (1H, s), 14.44 (1H, s) |
| 239 | 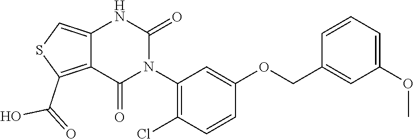 [SMILES: COc1cccc(COc2ccc(Cl)c(-n3c(=O)[nH]c4csc(C(=O)O)c4c3=O)c2)c1] | (DMSO-d6) 3.76 (3H, s), 5.08 (2H, s), 6.85-6.95 (1H, m), 7.0-7.05 (2H, m), 7.18 (1H, dd, J = 9.1 Hz, 3.1 Hz), 7.25- 7.35 (2H, m), 7.41 (1H, s), 7.57 (1H, d, J = 9.1 Hz), 12.04 (1H, s), 14.44 (1H, s) |
| 240 | 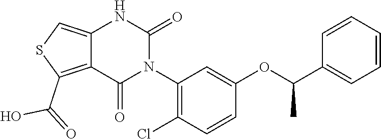 [SMILES: C[C@@H](Oc1ccc(Cl)c(-n2c(=O)[nH]c3csc(C(=O)O)c3c2=O)c1)c1ccccc1] | (DMSO-d6) 1.5-1.6 (3H, m), 5.45-5.55 (1H, m), 7.0-7.1 (1H, m), 7.23 (1H, dd, J = 6.1 Hz, 2.9 Hz), 7.25-7.5 (7H, m), 11.95-12.1 (1H, m), 14.42 (1H, s) |
| 241 | 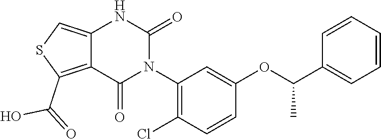 [SMILES: C[C@H](Oc1ccc(Cl)c(-n2c(=O)[nH]c3csc(C(=O)O)c3c2=O)c1)c1ccccc1] | (DMSO-d6) 1.5-1.6 (3H, m), 5.45-5.55 (1H, m), 7.0-7.1 (1H, m), 7.23 (1H, dd, J = 6.1 Hz, 2.9 Hz), 7.25-7.5 (7H, m), 11.95-12.1 (1H, m), 14.42 (1H, s) |
| 242 | 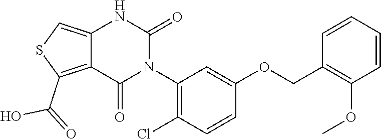 [SMILES: COc1ccccc1COc1ccc(Cl)c(-n2c(=O)[nH]c3csc(C(=O)O)c3c2=O)c1] | (DMSO-d6) 3.82 (3H, s), 5.04 (2H, s), 6.95-7.0 (1H, m), 7.06 (1H, d, J = 7.9 Hz), 7.17 (1H, dd, J = 9.1 Hz, 2.9 Hz), 7.3-7.45 (4H, m), 7.56 (1H, d, J = 9.1 Hz), 12.04 (1H, s), 14.46 (1H, s) |
| 243 | 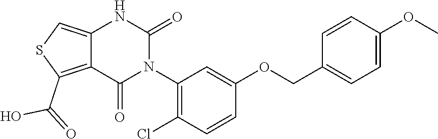 [SMILES: COc1ccc(COc2ccc(Cl)c(-n3c(=O)[nH]c4csc(C(=O)O)c4c3=O)c2)cc1] | (DMSO-d6) 3.76 (3H, s), 5.02 (2H, s), 6.9-7.0 (2H, m), 7.15-7.2 (1H, m), 7.3 (1H, d, J = 3.3 Hz), 7.35-7.45 (3H, m), 7.56 (1H, d, J = 9.0 Hz), 12.04 (1H, s), 14.46 (1H, s) |
| 244 | 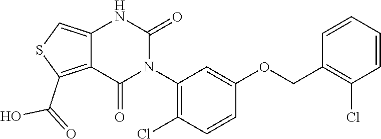 [SMILES: O=C(O)c1scc2[nH]c(=O)n(-c3cc(OCc4ccccc4Cl)ccc3Cl)c(=O)c12] | (DMSO-d6) 5.18 (2H, s), 7.02 (1H, s), 7.16 (1H, dd, J = 9.0 Hz, 2.7 Hz), 7.25- 7.4 (2H, m), 7.5-7.6 (2H, m), 7.8-7.9 (1H, m), 8.58 (1H, d, J = 4.5 Hz), 11.0- 12.5 (1H, br) |
| 245 | 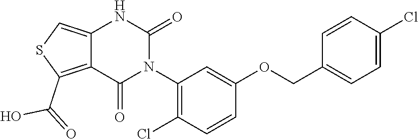 [SMILES: O=C(O)c1scc2[nH]c(=O)n(-c3cc(OCc4ccc(Cl)cc4)ccc3Cl)c(=O)c12] | (DMSO-d6) 5.11 (2H, s), 7.15-7.2 (1H, m), 7.32 (1H, d, J = 2.9 Hz), 7.41 (1H, s), 7.45-7.55 (4H, m), 7.57 (1H, d, J = 8.6 Hz), 12.06 (1H, s), 14.43 (1H, s) |
| 246 | 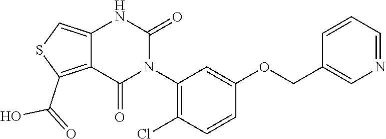 [SMILES: O=C(O)c1scc2[nH]c(=O)n(-c3cc(OCc4cccnc4)ccc3Cl)c(=O)c12] | (DMSO-d6) 5.16 (2H, s), 7.1-7.25 (2H, m), 7.31 (1H, d, J = 2.9 Hz), 7.4-7.5 (1H, m), 7.56 (1H, d, J = 9.0 Hz), 7.85-7.95 (1H, m), 8.5-8.6 (1H, m), 8.69 (1H, s), 11.0-13.0 (1H, br) |
| 247 | 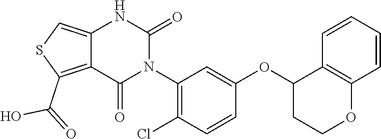 [SMILES: O=C(O)c1scc2[nH]c(=O)n(-c3cc(OC4CCOc5ccccc54)ccc3Cl)c(=O)c12] | (DMSO-d6) 2.1-2.25 (2H, m), 4.1-4.2 (1H, m), 4.25-4.35 (1H, m), 5.45-5.55 (1H, m), 6.8-6.95 (2H, m), 7.2-7.35 (3H, m), 7.35-7.45 (2H, m), 7.59 (1H, d, J = 8.7 Hz), 12.0-12.05 (1H, m), 14.42 (1H, s) |
| 248 | 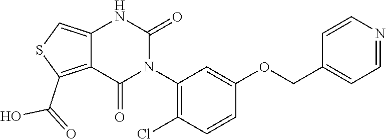 [SMILES: O=C(O)c1scc2[nH]c(=O)n(-c3cc(OCc4ccncc4)ccc3Cl)c(=O)c12] | (DMSO-d6) 5.2 (2H, s), 7.1-7.2 (2H, m), 7.3 (1H, d, J = 2.9 Hz), 7.4-7.5 (2H, m), 7.56 (1H, d, J = 8.8 Hz), 8.55-8.65 (2H, m), 1.10-13.0 (1H, br) |
| 249 | 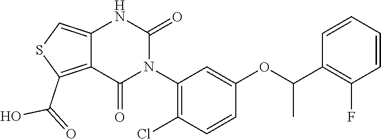 [SMILES: CC(Oc1ccc(Cl)c(-n2c(=O)[nH]c3csc(C(=O)O)c3c2=O)c1)c1ccccc1F] | (DMSO-d6) 1.6 (3H, d, J = 6.3 Hz), 5.65-5.75 (1H, m), 7.0-7.1 (1H, m), 7.15-7.3 (3H, m), 7.3-7.45 (2H, m), 7.45-7.55 (2H, m), 11.95-12.1 (1H, m), 14.42 (1H, s) |
| 250 | 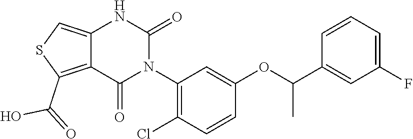 [SMILES: CC(Oc1ccc(Cl)c(-n2c(=O)[nH]c3csc(C(=O)O)c3c2=O)c1)c1cccc(F)c1] | (DMSO-d6) 1.5-1.6 (3H, m), 5.5-5.6 (1H, m), 7.0-7.15 (2H, m), 7.2-7.3 (3H, m), 7.35-7.55 (3H, m), 11.95-12.0 (1H, m), 14.41 (1H, s) |
| 251 | 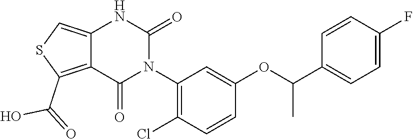 [SMILES: CC(Oc1ccc(Cl)c(-n2c(=O)[nH]c3csc(C(=O)O)c3c2=O)c1)c1ccc(F)cc1] | (DMSO-d6) 1.5-1.6 (3H, m), 5.45-5.6 (1H, m), 7.0-7.1 (1H, m), 7.15-7.25 (3H, m), 7.4 (1H, d, J = 4.1 Hz), 7.45-7.55 (3H, m), 11.95-12.05 (1H, m), 14.42 (1H, s) |
| 252 | 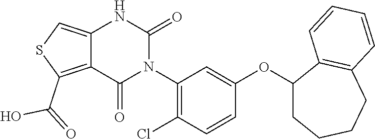 [SMILES: O=C(O)c1scc2[nH]c(=O)n(-c3cc(OC4CCCCc5ccccc54)ccc3Cl)c(=O)c12] | (DMSO-d6) 1.35-1.5 (1H, m), 1.65-2.05 (5H, m), 2.8-3.0 (2H, m), 5.45-5.6 (1H, m), 7.05-7.2 (4H, m), 7.25-7.35 (2H, m), 7.39 (1H, d, J = 3.8 Hz), 7.52 (1H, d, J = 8.8 Hz), 12.02 (1H, s), 14.43 (1H, s) |
| 253 | 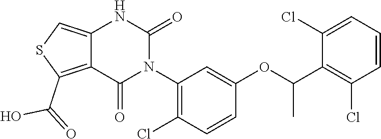 [SMILES: CC(Oc1ccc(Cl)c(-n2c(=O)[nH]c3csc(C(=O)O)c3c2=O)c1)c1c(Cl)cccc1Cl] | (DMSO-d6) 1.73 (3H, d, J = 6.6 Hz), 6.07 (1H, q, J = 6.6 Hz), 6.89 (1H, dd, J = 9.0 Hz, 3.0 Hz), 7.15-7.2 (1H, m), 7.3-7.4 (2H, m), 7.45-7.55 (3H, m), 12.0 (1H, s), 14.4 (1H, s) |
| 254 | 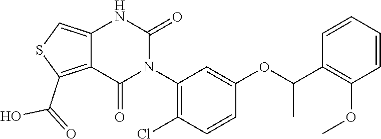 [SMILES: COc1ccccc1C(C)Oc1ccc(Cl)c(-n2c(=O)[nH]c3csc(C(=O)O)c3c2=O)c1] | (DMSO-d6) 1.53 (3H, d, J = 6.2 Hz), 5.68 (1H, q, J = 6.2 Hz), 6.85-7.0 (2H, m), 7.05 (1H, d, J = 8.4 Hz), 7.2 (1H, t, J = 3.2 Hz), 7.25-7.4 (3H, m), 7.47 (1H, d, J = 9.3 Hz), 12.01 (1H, s), 14.45 (1H, s) |
| 255 | 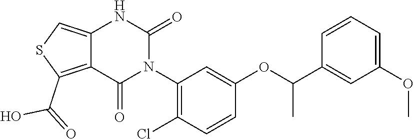 [SMILES: COc1cccc(C(C)Oc2ccc(Cl)c(-n3c(=O)[nH]c4csc(C(=O)O)c4c3=O)c2)c1] | (DMSO-d6) 1.5-1.6 (3H, m), 3.7-3.75 (3H, m), 5.4-5.5 (1H, m), 6.8-6.9 (1H, m), 6.95-7.1 (3H, m), 7.2-7.3 (2H, m), 7.4 (1H, d, J = 3.4 Hz), 7.47 (1H, dd, J = 9.0 Hz, 1.6 Hz), 11.95-12.05 (1H, m), 14.43 (1H, s) |
| 256 | 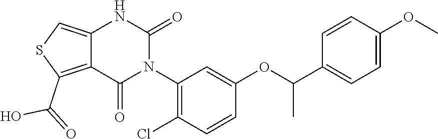 [SMILES: COc1ccc(C(C)Oc2ccc(Cl)c(-n3c(=O)[nH]c4csc(C(=O)O)c4c3=O)c2)cc1] | (DMSO-d6) 1.5-1.6 (3H, m), 3.73 (3H, s), 5.4-5.5 (1H, m), 6.85-6.95 (2H, m), 7.0-7.05 (1H, m), 7.15-7.25 (1H, m), 7.3-7.5 (4H, m), 11.95-12.05 (1H, m), 14.44 (1H, s) |
| 257 | 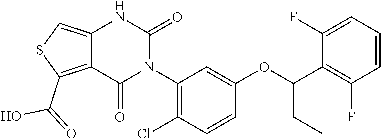 [SMILES: CCC(Oc1ccc(Cl)c(-n2c(=O)[nH]c3csc(C(=O)O)c3c2=O)c1)c1c(F)cccc1F] | (DMSO-d6) 0.93 (3H, t, J = 7.5 Hz), 1.9- 2.05 (1H, m), 2.1-2.25 (1H, m), 5.55 (1H, t, J = 7.3 Hz), 6.95-7.25 (4H, m), 7.35-7.5 (2H, m), 7.52 (1H, d, J = 8.7 Hz), 11.95-12.05 (1H, m), 14.4 (1H, s) |
| 258 | 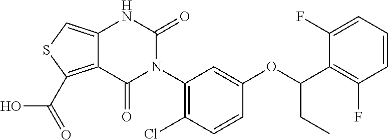 [SMILES: CCC(Oc1ccc(Cl)c(-n2c(=O)[nH]c3csc(C(=O)O)c3c2=O)c1)c1c(F)cccc1F] | (DMSO-d6) 0.93 (3H, t, J = 7.4 Hz), 1.9- 2.05 (1H, m), 2.1-2.25 (1H, m), 5.5-5.6 (1H, m), 6.95-7.25 (4H, m), 7.35-7.5 (1H, m), 7.52 (1H, d, J = 9.2 Hz), 7.95 (1H, d, J = 6.7 Hz), 12.99 (1H, s), 13.8- 14.0 (1H, m) |
| 259 | 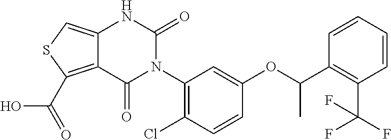 [SMILES: CC(Oc1ccc(Cl)c(-n2c(=O)[nH]c3csc(C(=O)O)c3c2=O)c1)c1ccccc1C(F)(F)F] | (DMSO-d6) 1.55-1.65 (3H, m), 5.65-5.8 (1H, m), 6.9-7.0 (1H, m), 7.2-7.3 (1H, m), 7.35-7.45 (1H, m), 7.45-7.6 (2H, m), 7.7-7.85 (3H, m), 12.03 (1H, s), 14.41 (1H, s) |
| 260 | 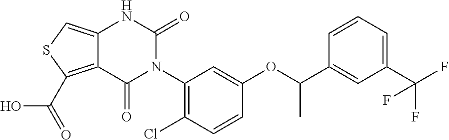 [SMILES: CC(Oc1ccc(Cl)c(-n2c(=O)[nH]c3csc(C(=O)O)c3c2=O)c1)c1cccc(C(F)(F)F)c1] | (DMSO-d6) 1.55-1.65 (3H, m), 5.6-5.7 (1H, m), 7.05-7.15 (1H, m), 7.25-7.3 (1H, m), 7.4 (1H, d, J = 5.6 Hz), 7.45- 7.55 (1H, m), 7.55-7.85 (4H, m), 11.95- 12.1 (1H, m), 14.4 (1H, s) |
| 261 | 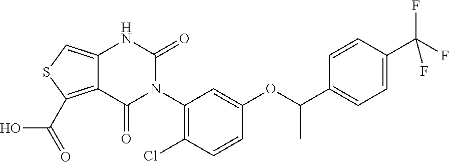 [SMILES: CC(Oc1ccc(Cl)c(-n2c(=O)[nH]c3csc(C(=O)O)c3c2=O)c1)c1ccc(C(F)(F)F)cc1] | (DMSO-d6) 1.55-1.65 (3H, m), 5.6-5.7 (1H, m), 7.0-7.1 (1H, m), 7.25 (1H, dd, J = 6.6 Hz, 3.0 Hz), 7.4 (1H, d, J = 5.3 Hz), 7.45-7.55 (1H, m), 7.6-7.7 (2H, m), 7.7-7.8 (2H, m), 11.95-12.1 (1H, m), 14.4 (1H, s) |
| 262 | 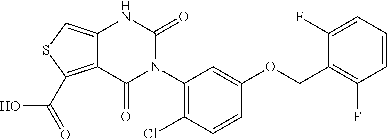 [SMILES: O=C(O)c1scc2[nH]c(=O)n(-c3cc(OCc4c(F)cccc4F)ccc3Cl)c(=O)c12] | (DMSO-d6) 5.13 (2H, s), 7.15-7.3 (3H, m), 7.34 (1H, d, J = 2.9 Hz), 7.4 (1H, s), 7.5-7.65 (2H, m), 12.03 (1H, s), 14.4 (1H, s) |
| 263 | 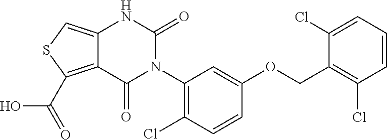 [SMILES: O=C(O)c1scc2[nH]c(=O)n(-c3cc(OCc4c(Cl)cccc4Cl)ccc3Cl)c(=O)c12] | (DMSO-d6) 5.24 (2H, s), 7.2-7.65 (7H, m), 12.02 (1H, s), 14.39 (1H, s) |
| 264 | 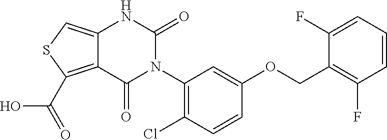 [SMILES: O=C(O)c1scc2[nH]c(=O)n(-c3cc(OCc4c(F)cccc4F)ccc3Cl)c(=O)c12] | (DMSO-d6) 5.12 (2H, s), 7.15-7.25 (3H, m), 7.32 (1H, d, J = 3.1 Hz), 7.5-7.65 (2H, m), 7.94 (1H, s), 12.8-13.2 (1H, br), 3.93 (1H, s) |
| 265 | 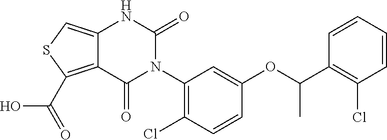 [SMILES: CC(Oc1ccc(Cl)c(-n2c(=O)[nH]c3csc(C(=O)O)c3c2=O)c1)c1ccccc1Cl] | (DMSO-d6) 1.59 (3H, d, J = 6.3 Hz), 5.71 (1H, q, J = 6.3 Hz), 6.85-6.95 (1H, m), 7.2-7.25 (1H, m), 7.3-7.45 (3H, m), 7.45-7.55 (3H, m), 11.95-12.05 (1H, m), 14.39 (1H, s) |
| 266 | 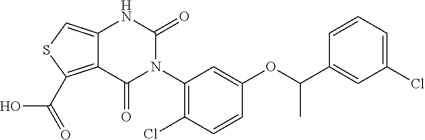 [SMILES: CC(Oc1ccc(Cl)c(-n2c(=O)[nH]c3csc(C(=O)O)c3c2=O)c1)c1cccc(Cl)c1] | (DMSO-d6) 1.56 (3H, d, J = 6.2 Hz), 5.45-5.6 (1H, m), 7.0-7.1 (1H, m), 7.2- 7.55 (7H, m), 11.95-12.05 (1H, m), 14.39 (1H, s) |
| 267 | 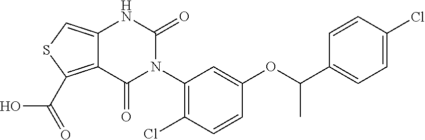 [SMILES: CC(Oc1ccc(Cl)c(-n2c(=O)[nH]c3csc(C(=O)O)c3c2=O)c1)c1ccc(Cl)cc1] | (DMSO-d6) 1.5-1.6 (3H, m), 5.45-5.6 (1H, m), 7.0-7.05 (1H, m), 7.22 (1H, dd, J = 8.9 Hz, 3.0 Hz), 7.35-7.5 (6H, m), 11.9-12.05 (1H, m), 14.39 (1H, s) |
| 268 | 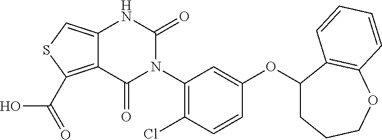 [SMILES: O=C(O)c1scc2[nH]c(=O)n(-c3cc(OC4CCCOc5ccccc54)ccc3Cl)c(=O)c12] | (DMSO-d6) 1.9-2.15 (4H, m), 3.75-3.85 (1H, m), 4.1-4.25 (1H, m), 5.45-5.55 (1H, m), 6.95-7.45 (7H, m), 7.53 (1H, d, J = 8.9 Hz), 11.95-12.05 (1H, m), 14.42 (1H, s) |
| 269 | 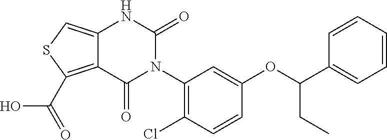 [SMILES: CCC(Oc1ccc(Cl)c(-n2c(=O)[nH]c3csc(C(=O)O)c3c2=O)c1)c1ccccc1] | (DMSO-d6) 0.91 (3H, t, J = 7.5 Hz), 1.75-2.0 (2H, m), 5.2-5.3 (1H, m), 6.95- 7.05 (1H, m), 7.15-7.5 (8H, m), 11.95- 12.05 (1H, m), 14.39 (1H, s) |
| 270 | 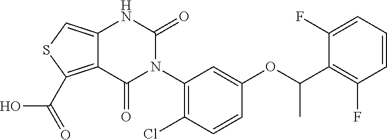 [SMILES: CC(Oc1ccc(Cl)c(-n2c(=O)[nH]c3csc(C(=O)O)c3c2=O)c1)c1c(F)cccc1F] | (DMSO-d6) 1.71 (3H, d, J = 6.5 Hz), 5.81 (1H, q, J = 6.5 Hz), 7.0-7.15 (3H, m), 7.2-7.25 (1H, m), 7.35-7.5 (2H, m), 7.53 (1H, d, J = 9.2 Hz), 11.95-12.05 (1H, m), 14.41 (1H, s) |
| 271 | 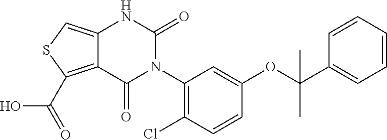 [SMILES: CC(C)(Oc1ccc(Cl)c(-n2c(=O)[nH]c3csc(C(=O)O)c3c2=O)c1)c1ccccc1] | (DMSO-d6) 1.65-1.75 (6H, m), 6.64 (1H, dd, J = 8.7 Hz, 2.8 Hz), 7.07 (1H, d, J = 2.8 Hz), 7.25-7.5 (7H, m), 11.97 (1H, s), 14.42 (1H, s) |
| 272 | 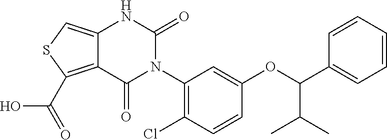 [SMILES: CC(C)C(Oc1ccc(Cl)c(-n2c(=O)[nH]c3csc(C(=O)O)c3c2=O)c1)c1ccccc1] | (DMSO-d6) 0.82 (3H, d, J = 6.4 Hz), 0.95-1.05 (3H, m), 2.05-2.15 (1H, m), 5.0-5.1 (1H, m), 6.95-7.05 (1H, m), 7.15-7.5 (8H, m), 11.95-12.05 (1H, m), 14.4 (1H, s) |
| 273 | 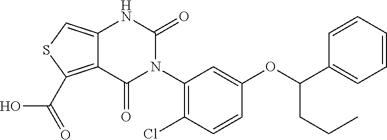 [SMILES: CCCC(Oc1ccc(Cl)c(-n2c(=O)[nH]c3csc(C(=O)O)c3c2=O)c1)c1ccccc1] | (DMSO-d6) 0.91 (3H, t, J = 7.4 Hz), 1.25-1.5 (2H, m), 1.7-1.8 (1H, m), 1.85- 2.0 (1H, m), 5.3-5.35 (1H, m), 6.95-7.05 (1H, m), 7.15-7.5 (8H, m), 11.95-12.05 (1H, m), 14.41 (1H, brs) |
| 274 | 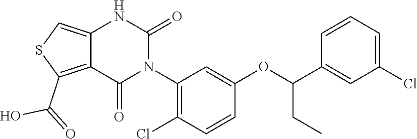 [SMILES: CCC(Oc1ccc(Cl)c(-n2c(=O)[nH]c3csc(C(=O)O)c3c2=O)c1)c1cccc(Cl)c1] | (DMSO-d6) 0.91 (3H, t, J = 7.4 Hz), 1.75-2.0 (2H, m), 5.25-5.35 (1H, m), 7.0-7.1 (1H, m), 7.2-7.25 (1H, m), 7.3- 7.55 (6H, m), 11.95-12.05 (1H, m), 14.4 (1H, s) |
| 275 | 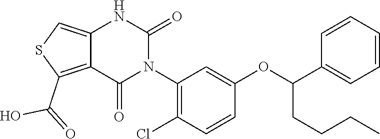 [SMILES: CCCCC(Oc1ccc(Cl)c(-n2c(=O)[nH]c3csc(C(=O)O)c3c2=O)c1)c1ccccc1] | (DMSO-d6) 0.85 (3H, t, J = 7.1 Hz), 1.2- 1.45 (4H, m), 1.7-1.85 (1H, m), 1.9-2.0 (1H, m), 5.25-5.35 (1H, m), 6.95-7.05 (1H, m), 7.15-7.5 (8H, m), 11.95-12.05 (1H, m), 14.41 (1H, s) |
| 276 | 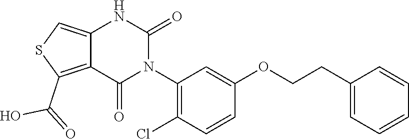 [SMILES: O=C(O)c1scc2[nH]c(=O)n(-c3cc(OCCc4ccccc4)ccc3Cl)c(=O)c12] | (DMSO-d6) 3.05 (2H, t, J = 7.0 Hz), 4.19 (2H, t, J = Hz), 7.05-7.15 (1H, m), 7.2-7.35 (6H, m), 7.4 (1H, s), 7.54 (1H, d, J = 9.2 Hz), 12.03 (1H, s), 14.45 (1H, s) |
| 277 | 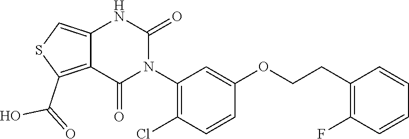 [SMILES: O=C(O)c1scc2[nH]c(=O)n(-c3cc(OCCc4ccccc4F)ccc3Cl)c(=O)c12] | (DMSO-d6) 3.09 (2H, t, J = 6.8 Hz), 4.19 (2H, t, J = 6.8 Hz), 7.05-7.35 (5H, m), 7.35-7.45 (2H, m), 7.54 (1H, d, J = 8.9 Hz), 12.03 (1H, s), 14.45 (1H, s) |
| 278 | 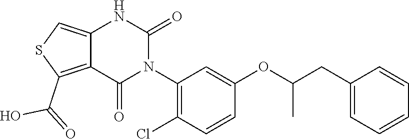 [SMILES: CC(Cc1ccccc1)Oc1ccc(Cl)c(-n2c(=O)[nH]c3csc(C(=O)O)c3c2=O)c1] | (DMSO-d6) 1.22 (3H, d, J = 6.0 Hz), 2.8-2.9 (1H, m), 2.95-3.05 (1H, m), 4.6- 4.75 (1H, m), 7.09 (1H, dd, J = 9.0 Hz, 3.0 Hz), 7.15-7.35 (6H, m), 7.35-7.45 (1H, m), 7.52 (1H, d, J = 9.2 Hz), 12.02 (1H, s), 14.46 (1H, s) |
| 279 | 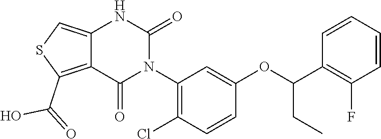 [SMILES: CCC(Oc1ccc(Cl)c(-n2c(=O)[nH]c3csc(C(=O)O)c3c2=O)c1)c1ccccc1F] | (DMSO-d6) 0.93 (3H, t, J = 7.5 Hz), 1.8- 2.05 (2H, m), 5.47 (1H, t, J = 6.1 Hz), 6.95-7.05 (1H, m), 7.15-7.25 (3H, m), 7.3-7.55 (4H, m), 11.95-12.05 (1H, m), 14.35-14.45 (1H, m) |
| 280 | 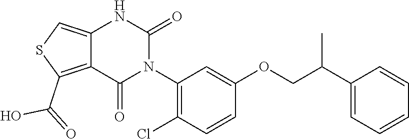 [SMILES: CC(COc1ccc(Cl)c(-n2c(=O)[nH]c3csc(C(=O)O)c3c2=O)c1)c1ccccc1] | (DMSO-d6) 1.32 (3H, d, J = 6.9 Hz), 3.15-3.3 (1H, m), 4.0-4.15 (2H, m), 7.09 (1H, dd, J = 8.9 Hz, 3.1 Hz), 7.15-7.45 (7H, m), 7.52 (1H, d, J = 8.9 Hz), 12.02 (1H, s), 14.45 (1H, s) |
| 281 | 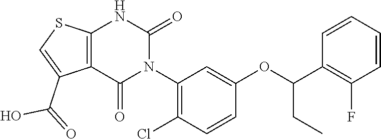 [SMILES: CCC(Oc1ccc(Cl)c(-n2c(=O)[nH]c3scc(C(=O)O)c3c2=O)c1)c1ccccc1F] | (DMSO-d6) 0.93 (3H, t, J = 7.5 Hz), 1.8- 2.05 (2H, m), 5.48 (1H, t, J = 6.5 Hz), 6.95-7.05 (1H, m), 7.15-7.25 (3H, m), 7.3-7.4 (1H, m), 7.4-7.55 (2H, m), 7.94 (1H, d, J = 3.4 Hz), 12.98 (1H, s), 13.93 (1H, s) |
| 282 | 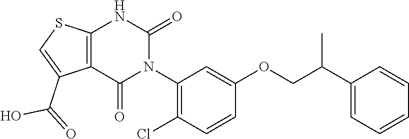 [SMILES: CC(COc1ccc(Cl)c(-n2c(=O)[nH]c3scc(C(=O)O)c3c2=O)c1)c1ccccc1] | (DMSO-d6) 1.32 (3H, d, J = 6.9 Hz), 3.15-3.3 (1H, m), 4.0-4.15 (2H, m), 7.05-7.1 (1H, m), 7.15-7.4 (6H, m), 7.52 (1H, d, J = 9.0 Hz), 7.95 (1H, s), 13.0 (1H, s), 13.98 (1H, s) |
| 283 | 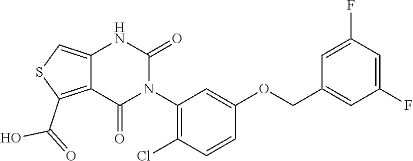 [SMILES: O=C(O)c1scc2[nH]c(=O)n(-c3cc(OCc4cc(F)cc(F)c4)ccc3Cl)c(=O)c12] | (DMSO-d6) 5.15 (2H, s), 7.15-7.3 (4H, m), 7.35 (1H, d, J = 2.8 Hz), 7.46 (1H, s), 7.59 (1H, d, J = 8.8 Hz), 12.19 (1H, s) |
| 284 | 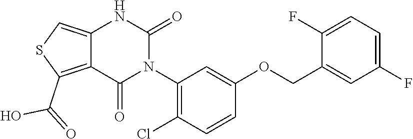 [SMILES: O=C(O)c1scc2[nH]c(=O)n(-c3cc(OCc4cc(F)ccc4F)ccc3Cl)c(=O)c12] | (DMSO-d6) 5.14 (2H, s), 7.2-7.5 (6H, m), 7.6 (1H, d, J = 8.7 Hz), 12.11 (1H, s), 14.44 (1H, s) |
| 285 | 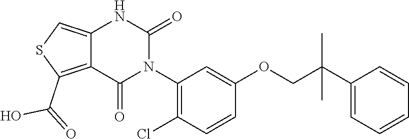 [SMILES: CC(C)(COc1ccc(Cl)c(-n2c(=O)[nH]c3csc(C(=O)O)c3c2=O)c1)c1ccccc1] | (DMSO-d6) 1.4 (6H, s), 4.01 (2H, s), 7.05-7.15 (1H, m), 7.15-7.25 (2H, m), 7.3-7.35 (2H, m), 7.39 (1H, s), 7.4-7.5 (2H, m), 7.52 (1H, d, J = 9.2 Hz), 12.0 (1H, s), 14.44 (1H, s) |
| 286 | 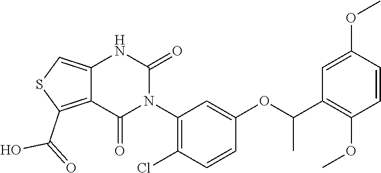 [SMILES: COc1ccc(OC)c(C(C)Oc2ccc(Cl)c(-n3c(=O)[nH]c4csc(C(=O)O)c4c3=O)c2)c1] | (DMSO-d6) 1.52 (3H, d, J = 6.3 Hz), 3.67 (3H, s), 3.75-3.85 (3H, m), 5.65 (1H, q, J = 6.3 Hz), 6.8-7.0 (4H, m), 7.15-7.25 (1H, m), 7.39 (1H, d, J = 1.9 Hz), 7.48 (1H, d, J = 8.9 Hz), 11.99 (1H, s), 14.42 (1H, s) |
| 287 | 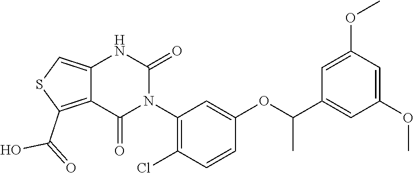 [SMILES: COc1cc(OC)cc(C(C)Oc2ccc(Cl)c(-n3c(=O)[nH]c4csc(C(=O)O)c4c3=O)c2)c1] | (DMSO-d6) 1.55 (3H, d, J = 6.3 Hz), 3.31 (3H, s), 3.7-3.75 (3H, m), 5.35-5.45 (1H, m), 6.35-6.45 (1H, m), 6.5-6.6 (2H, m), 7.0-7.1 (1H, m), 7.2-7.3 (1H, m), 7.39 (1H, d, J = 2.0 Hz), 7.45-7.5 (1H, m), 11.95-12.05 (1H, m), 14.41 (1H, s) |
| 288 | 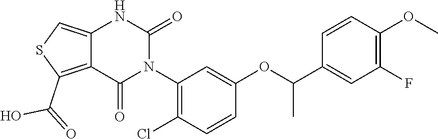 [SMILES: COc1ccc(C(C)Oc2ccc(Cl)c(-n3c(=O)[nH]c4csc(C(=O)O)c4c3=O)c2)cc1F] | (DMSO-d6) 1.5-1.6 (3H, m), 3.81 (3H, s), 5.4-5.5 (1H, m), 7.0-7.3 (5H, m), 7.4 (1H, d, J = 3.9 Hz), 7.48 (1H, dd, J = 8.9 Hz, 1.6 Hz), 11.95-12.05 (1H, m), 14.41 (1H, s) |
| 289 | 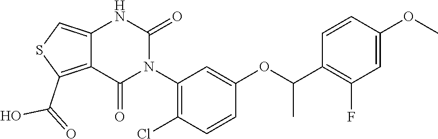 [SMILES: COc1ccc(C(C)Oc2ccc(Cl)c(-n3c(=O)[nH]c4csc(C(=O)O)c4c3=O)c2)c(F)c1] | (DMSO-d6) 1.58 (3H, d, J = 6.4 Hz), 3.75 (3H, s), 5.55-5.7 (1H, m), 6.75-6.9 (2H, m), 6.95-7.05 (1H, m), 7.15-7.55 (4H, m), 11.95-12.05 (1H, m), 14.42 (1H, s) |
| 290 | 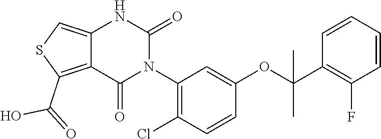 [SMILES: CC(C)(Oc1ccc(Cl)c(-n2c(=O)[nH]c3csc(C(=O)O)c3c2=O)c1)c1ccccc1F] | (DMSO-d6) 1.75 (6H, s), 6.78 (1H, dd, J = 9.0 Hz, 3.0 Hz), 7.0-7.55 (7H, m), 11.91 (1H, brs), 14.0-14.8 (1H, br) |
| 291 | 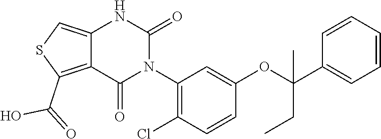 [SMILES: CCC(C)(Oc1ccc(Cl)c(-n2c(=O)[nH]c3csc(C(=O)O)c3c2=O)c1)c1ccccc1] | (DMSO-d6) 0.8 (3H, t, J = 7.3 Hz), 1.65- 1.7 (3H, m), 1.85-2.05 (2H, m), 6.55- 6.65 (1H, m), 7.08 (1H, dd, J = 6.6 Hz, 3.0 Hz), 7.25-7.45 (7H, m), 11.97 (1H, s), 14.43 (1H, s) |
| 292 | 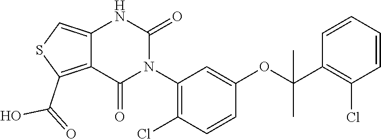 [SMILES: CC(C)(Oc1ccc(Cl)c(-n2c(=O)[nH]c3csc(C(=O)O)c3c2=O)c1)c1ccccc1Cl] | (DMSO-d6) 1.83 (6H, s), 6.69 (1H, dd, J = 9.1 Hz, 3.1 Hz), 7.06 (1H, d, J = 3.1 Hz), 7.3-7.5 (5H, m), 7.61 (1H, dd, J = 7.7 Hz, 1.8 Hz), 11.96 (1H, s), 14.42 (1H, s) |
| 293 | 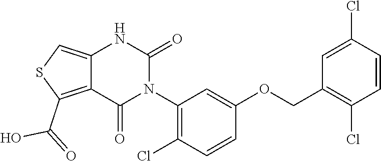 [SMILES: O=C(O)c1scc2[nH]c(=O)n(-c3cc(OCc4cc(Cl)ccc4Cl)ccc3Cl)c(=O)c12] | (DMSO-d6) 5.15 (2H, s), 7.15-7.75 (7H, m), 12.03 (1H, s), 14.41 (1H, s) |
| 294 | 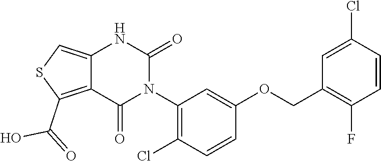 [SMILES: O=C(O)c1scc2[nH]c(=O)n(-c3cc(OCc4cc(Cl)ccc4F)ccc3Cl)c(=O)c12] | (DMSO-d6) 5.14 (2H, s), 7.23 (1H, dd, J = 8.8 Hz, 3.2 Hz), 7.3-7.45 (3H, m), 7.45-7.55 (1H, m), 7.59 (1H, d, J = 9.1 Hz), 7.65-7.7 (1H, m), 12.03 (1H, s), 14.41 (1H, s) |
| 295 | 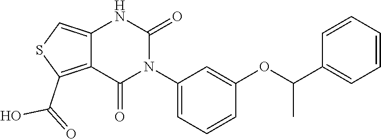 [SMILES: CC(Oc1cccc(-n2c(=O)[nH]c3csc(C(=O)O)c3c2=O)c1)c1ccccc1] | (DMSO-d6) 1.56 (3H, d, J = 6.3 Hz), 5.45-5.55 (1H, m), 6.85-6.9 (1H, m), 6.95-7.05 (2H, m), 7.25-7.45 (7H, m), 11.9 (1H, s) |
| 296 | 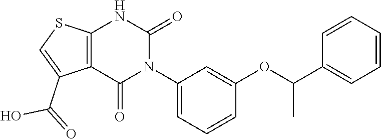 [SMILES: CC(Oc1cccc(-n2c(=O)[nH]c3scc(C(=O)O)c3c2=O)c1)c1ccccc1] | (DMSO-d6) 1.56 (3H, d, J = 6.4 Hz), 5.5 (1H, q, J = 6.4 Hz), 6.85-6.9 (1H, m), 6.9-7.05 (2H, m), 7.25-7.4 (4H, m), 7.4- 7.45 (2H, m), 7.99 (1H, s), 12.86 (1H, s), 14.43 (1H, s) |
| 297 | 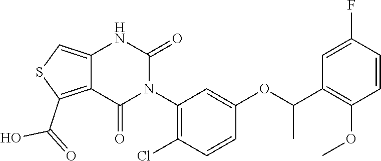 [SMILES: COc1ccc(F)cc1C(C)Oc1ccc(Cl)c(-n2c(=O)[nH]c3csc(C(=O)O)c3c2=O)c1] | (DMSO-d6) 1.52 (3H, d, J = 6.0 Hz), 3.84 (3H, s), 5.6-5.7 (1H, m), 6.85-7.55 (7H, m), 11.99 (1H, s), 14.42 (1H, s) |
| 298 | 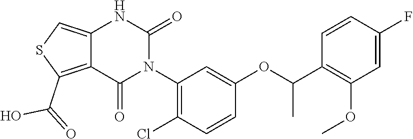 [SMILES: COc1cc(F)ccc1C(C)Oc1ccc(Cl)c(-n2c(=O)[nH]c3csc(C(=O)O)c3c2=O)c1] | (DMSO-d6) 1.52 (3H, d, J = 6.4 Hz), 3.85-3.9 (3H, m), 5.6-5.7 (1H, m), 6.75- 6.8 (1H, m), 6.85-7.0 (2H, m), 7.15-7.25 (1H, m), 7.3-7.45 (2H, m), 7.48 (1H, d, J = 9.2 Hz), 12.0 (1H, s), 14.44 (1H, s) |
| 299 | 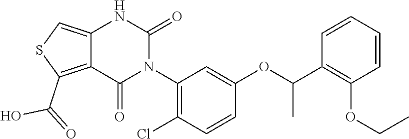 [SMILES: CCOc1ccccc1C(C)Oc1ccc(Cl)c(-n2c(=O)[nH]c3csc(C(=O)O)c3c2=O)c1] | (DMSO-d6) 1.3-1.4 (3H, m), 1.54 (3H, d, J = 6.3 Hz), 4.05-4.2 (2H, m), 5.65-5.75 (1H, m), 6.85-7.0 (2H, m), 7.0-7.05 (1H, m), 7.15-7.35 (3H, m), 7.39 (1H, d, J = 4.2 Hz), 7.48 (1H, d, J = 9.1 Hz), 11.95- 12.05 (1H, m), 14.42 (1H, s) |
| 300 | 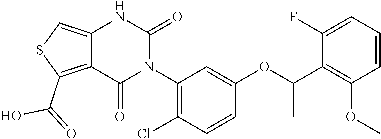 [SMILES: COc1cccc(F)c1C(C)Oc1ccc(Cl)c(-n2c(=O)[nH]c3csc(C(=O)O)c3c2=O)c1] | (DMSO-d6) 1.66 (3H, d, J = 6.6 Hz), 3.85-3.9 (3H, m), 5.8-5.9 (1H, m), 6.75- 6.85 (1H, m), 6.85-7.0 (2H, m), 7.17 (1H, d, J = 2.6 Hz), 7.25-7.4 (2H, m), 7.48 (1H, d, J = 8.6 Hz), 12.0 (1H, s), 14.42 (1H, s) |
| 301 | 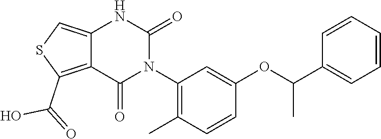 [SMILES: Cc1ccc(OC(C)c2ccccc2)cc1-n1c(=O)[nH]c2csc(C(=O)O)c2c1=O] | (DMSO-d6) 1.5-1.6 (3H, m), 1.95 (3H, s), 5.4-5.5 (1H, m), 6.85-6.95 (1H, m), 6.98 (1H, dd, J = 5.8 Hz, 2.6 Hz), 7.15- 7.45 (7H, m), 11.85-11.95 (1H, m), 14.76 (1H, s) |
| 302 | 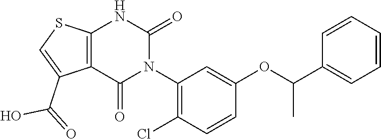 [SMILES: CC(Oc1ccc(Cl)c(-n2c(=O)[nH]c3scc(C(=O)O)c3c2=O)c1)c1ccccc1] | (DMSO-d6) 1.56 (3H, d, J = 6.3 Hz), 5.52 (1H, q, J = 6.3 Hz), 7.03 (1H, dd, J = 9.0 Hz, 3.0 Hz), 7.15-7.5 (7H, m), 7.9-8.0 (1H, m), 12.99 (1H, brs), 13.95 (1H, s) |
| 303 | 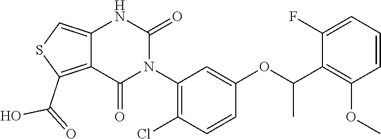 [SMILES: COc1cccc(F)c1C(C)Oc1ccc(Cl)c(-n2c(=O)[nH]c3csc(C(=O)O)c3c2=O)c1] | (DMSO-d6) 3.85 (3H, s), 5.03 (2H, s), 6.85-7.0 (2H, m), 7.21 (1H, dd, J = 8.9 Hz, 3.1 Hz), 7.32 (1H, d, J = 3.1 Hz), 7.4-7.5 (2H, m), 7.58 (1H, d, J = 8.9 Hz), 12.06 (1H, s), 14.43 (1H, s) |
| 304 | 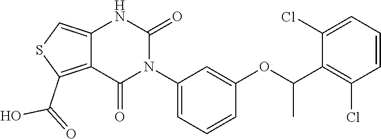 [SMILES: CC(Oc1cccc(-n2c(=O)[nH]c3csc(C(=O)O)c3c2=O)c1)c1c(Cl)cccc1Cl] | (DMSO-d6) 1.73 (3H, d, J = 6.7 Hz), 6.0-6.1 (1H, m), 6.8-6.85 (1H, m), 6.9- 7.0 (2H, m), 7.3-7.4 (3H, m), 7.45-7.5 (2H, m), 11.89 (1H, s), 14.86 (1H, s) |
| 305 | 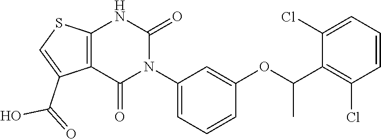 [SMILES: CC(Oc1cccc(-n2c(=O)[nH]c3scc(C(=O)O)c3c2=O)c1)c1c(Cl)cccc1Cl] | (DMSO-d6) 1.73 (3H, d, J = 6.7 Hz), 6.05 (1H, q, J = 6.7 Hz), 6.8-7.0 (3H, m), 7.3-7.4 (2H, m), 7.45-7.5 (2H, m), 7.99 (1H, s), 12.92 (1H, s), 14.43 (1H, s) |
| 306 | 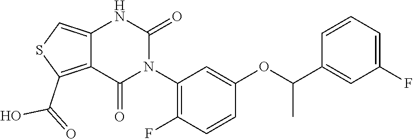 [SMILES: CC(Oc1ccc(F)c(-n2c(=O)[nH]c3csc(C(=O)O)c3c2=O)c1)c1cccc(F)c1] | (DMSO-d6) 1.56 (3H, d, J = 5.7 Hz), 5.45-5.55 (1H, m), 7.0-7.5 (8H, m), 11.95-12.05 (1H, m), 14.4 (1H, brs) |
| 307 | 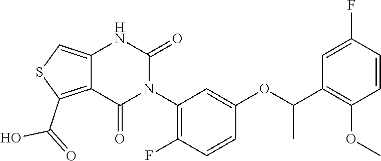 [SMILES: COc1ccc(F)cc1C(C)Oc1ccc(F)c(-n2c(=O)[nH]c3csc(C(=O)O)c3c2=O)c1] | (DMSO-d6) 1.52 (3H, d, J = 6.2 Hz), 3.84 (3H, s), 5.55-5.65 (1H, m), 6.9-7.0 (1H, m), 7.0-7.15 (4H, m), 7.2-7.3 (1H, m), 7.35-7.4 (1H, m), 11.98 (1H, s), 14.4 (1H, s) |
| 308 | 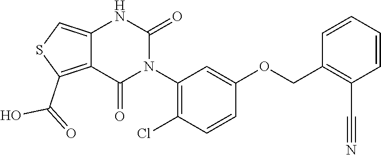 [SMILES: N#Cc1ccccc1COc1ccc(Cl)c(-n2c(=O)[nH]c3csc(C(=O)O)c3c2=O)c1] | (DMSO-d6) 5.2-5.35 (2H, m), 7.25 (1H, dd, J = 9.1 Hz, 3.0 Hz), 7.38 (1H, d, J = 3.0 Hz), 7.41 (1H, s), 7.55-7.65 (2H, m), 7.75-7.8 (2H, m), 7.94 (1H, d, J = 7.7 Hz), 12.05 (1H, s), 14.43 (1H, s) |
| 309 | 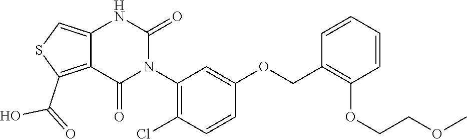 [SMILES: COCCOc1ccccc1COc1ccc(Cl)c(-n2c(=O)[nH]c3csc(C(=O)O)c3c2=O)c1] | (DMSO-d6) 3.26 (3H, s), 3.6-3.7 (2H, m), 4.1-4.2 (2H, m), 5.07 (2H, s), 6.95- 7.0 (1H, m), 7.07 (1H, d, J = 8.0 Hz), 7.15-7.2 (1H, m), 7.3-7.4 (2H, m), 7.4- 7.45 (2H, m), 7.55 (1H, d, J = 9.3 Hz), 12.04 (1H, s), 14.46 (1H, s) |
| 310 | 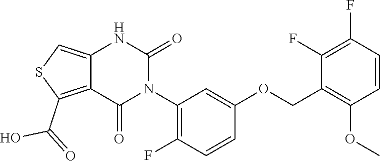 [SMILES: COc1ccc(F)c(F)c1COc1ccc(F)c(-n2c(=O)[nH]c3csc(C(=O)O)c3c2=O)c1] | (DMSO-d6) 3.83 (3H, s), 5.0-5.1 (2H, m), 6.9-6.95 (1H, m), 7.15-7.25 (2H, m), 7.3-7.4 (2H, m), 7.45-7.55 (1H, m), 12.02 (1H, s), 14.4 (1H, s) |
| 311 | 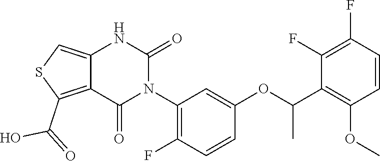 [SMILES: COc1ccc(F)c(F)c1C(C)Oc1ccc(F)c(-n2c(=O)[nH]c3csc(C(=O)O)c3c2=O)c1] | (DMSO-d6) 1.66 (3H, d, J = 6.5 Hz), 3.8-3.9 (3H, m), 5.75-5.85 (1H, m), 6.8- 7.0 (2H, m), 7.05-7.15 (1H, m), 7.2-7.4 (3H, m), 11.95-12.05 (1H, m), 14.39 (1H, s) |
| 312 | 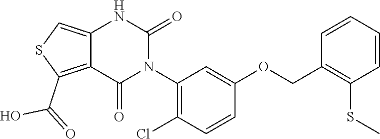 [SMILES: CSc1ccccc1COc1ccc(Cl)c(-n2c(=O)[nH]c3csc(C(=O)O)c3c2=O)c1] | (DMSO-d6) 2.48 (3H, s), 5.09 (2H, s), 7.15-7.25 (2H, m), 7.34 (1H, d, J = 2.9 Hz), 7.35-7.45 (3H, m), 7.49 (1H, d, J = 7.2 Hz), 7.58 (1H, d, J = 9.0 Hz), 12.04 (1H, s), 14.45 (1H, s) |
| 313 | 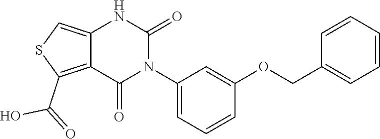 [SMILES: O=C(O)c1scc2[nH]c(=O)n(-c3cccc(OCc4ccccc4)c3)c(=O)c12] | (DMSO-d6) 5.1 (2H, s), 6.95-7.0 (1H, m), 7.05-7.15 (2H, m), 7.3-7.5 (7H, m), 11.88 (1H, s), 14.89 (1H, s) |
| 314 | 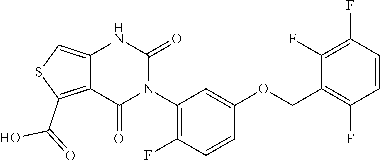 [SMILES: O=C(O)c1scc2[nH]c(=O)n(-c3cc(OCc4c(F)ccc(F)c4F)ccc3F)c(=O)c12] | (DMSO-d6) 5.14 (2H, s), 7.2-7.3 (3H, m), 7.35-7.45 (2H, m), 7.55-7.65 (1H, m), 12.02 (1H, s), 14.38 (1H, s) |
| 315 | 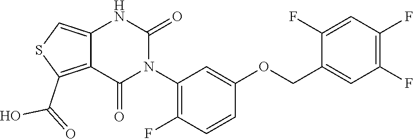 [SMILES: O=C(O)c1scc2[nH]c(=O)n(-c3cc(OCc4cc(F)c(F)cc4F)ccc3F)c(=O)c12] | (DMSO-d6) 5.08 (2H, s), 7.15-7.25 (1H, m), 7.25-7.3 (1H, m), 7.35-7.4 (2H, m), 7.55-7.7 (1H, m), 7.7-7.8 (1H, m), 12.0 (1H, s), 14.4 (1H, s) |
| 316 | 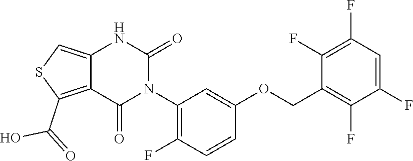 [SMILES: O=C(O)c1scc2[nH]c(=O)n(-c3cc(OCc4c(F)c(F)cc(F)c4F)ccc3F)c(=O)c12] | (DMSO-d6) 5.2 (2H, s), 7.2-7.3 (2H, m), 7.35-7.45 (2H, m), 7.95-8.05 (1H, m), 12.02 (1H, s), 14.38 (1H, s) |
| 317 | 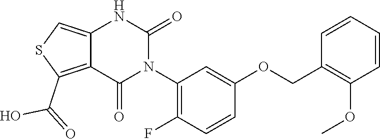 [SMILES: COc1ccccc1COc1ccc(F)c(-n2c(=O)[nH]c3csc(C(=O)O)c3c2=O)c1] | (DMSO-d6) 3.82 (3H, s), 5.02 (2H, s), 6.95-7.0 (1H, m), 7.06 (1H, d, J = 8.2 Hz), 7.1-7.2 (1H, m), 7.24 (1H, dd, J = 6.0 Hz, 3.2 Hz), 7.3-7.45 (4H, m), 12.01 (1H, s), 14.44 (1H, s) |
| 318 | 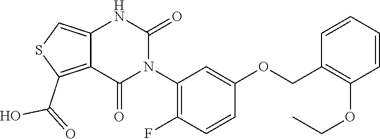 [SMILES: CCOc1ccccc1COc1ccc(F)c(-n2c(=O)[nH]c3csc(C(=O)O)c3c2=O)c1] | (DMSO-d6) 1.31 (3H, t, J = 6.9 Hz), 4.09 (2H, q, J = 6.9 Hz), 5.03 (2H, s), 6.9-7.0 (1H, m), 7.04 (1H, d, J = 8.2 Hz), 7.1-7.2 (1H, m), 7.2-7.25 (1H, m), 7.3- 7.45 (4H, m), 12.01 (1H, s), 14.43 (1H, s) |
| 319 | 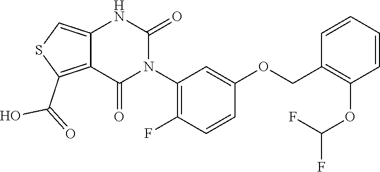 [SMILES: O=C(O)c1scc2[nH]c(=O)n(-c3cc(OCc4ccccc4OC(F)F)ccc3F)c(=O)c12] | (DMSO-d6) 5.08 (2H, s), 7.05-7.5 (7H, m), 7.55-7.65 (1H, m), 12.01 (1H, s), 14.42 (1H, s) |
| 320 | 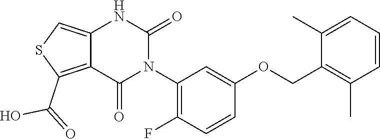 [SMILES: Cc1cccc(C)c1COc1ccc(F)c(-n2c(=O)[nH]c3csc(C(=O)O)c3c2=O)c1] | (DMSO-d6) 2.34 (6H, s), 6.03 (2H, s), 7.0-7.45 (7H, m), 12.0 (1H, s), 14.4 (1H, s) |
| 321 | 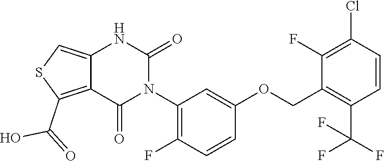 [SMILES: O=C(O)c1scc2[nH]c(=O)n(-c3cc(OCc4c(C(F)(F)F)ccc(Cl)c4F)ccc3F)c(=O)c12] | (DMSO-d6) 5.17 (2H, s), 7.2-7.3 (2H, m), 7.35-7.45 (2H, m), 7.74 (1H, d, J = 8.2 Hz), 7.9-8.0 (1H, m), 12.02 (1H, s), 14.38 (1H, s) |
| 322 | 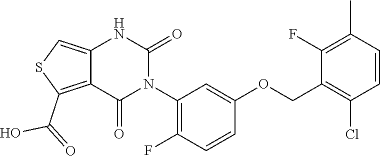 [SMILES: Cc1ccc(Cl)c(COc2ccc(F)c(-n3c(=O)[nH]c4csc(C(=O)O)c4c3=O)c2)c1F] | (DMSO-d6) 2.2-2.3 (3H, m), 5.05-5.15 (2H, m), 7.2-7.3 (2H, m), 7.32 (1H, d, J = 8.6 Hz), 7.35-7.45 (3H, m), 12.02 (1H, s), 14.39 (1H, s) |
| 323 | 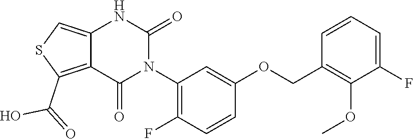 [SMILES: COc1c(F)cccc1COc1ccc(F)c(-n2c(=O)[nH]c3csc(C(=O)O)c3c2=O)c1] | (DMSO-d6) 3.85-3.95 (3H, m), 5.07 (2H, s), 7.1-7.4 (7H, m), 12.01 (1H, s), 14.42 (1H, s) |
| 324 | 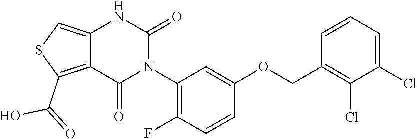 [SMILES: O=C(O)c1scc2[nH]c(=O)n(-c3cc(OCc4cccc(Cl)c4Cl)ccc3F)c(=O)c12] | (DMSO-d6) 5.18 (2H, s), 7.15-7.25 (1H, m), 7.25-7.35 (1H, m), 7.35-7.5 (3H, m), 7.55-7.7 (2H, m), 12.01 (1H, s), 14.41 (1H, s) |
| 325 | 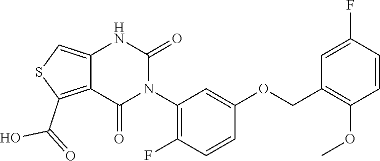 [SMILES: COc1ccc(F)cc1COc1ccc(F)c(-n2c(=O)[nH]c3csc(C(=O)O)c3c2=O)c1] | (DMSO-d6) 3.81 (3H, s), 5.02 (2H, s), 7.0-7.1 (1H, m), 7.1-7.2 (2H, m), 7.2- 7.4 (4H, m), 12.01 (1H, s), 14.42 (1H, s) |
| 326 | 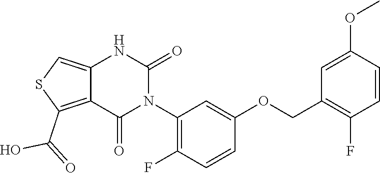 [SMILES: COc1ccc(F)c(COc2ccc(F)c(-n3c(=O)[nH]c4csc(C(=O)O)c4c3=O)c2)c1] | (DMSO-d6) 3.75 (3H, s), 5.08 (2H, s), 6.9-7.0 (1H, m), 7.1-7.25 (3H, m), 7.27 (1H, dd, J = 6.1 Hz, 3.2 Hz), 7.3-7.4 (2H, m), 12.02 (1H, s), 14.41 (1H, s) |
| 327 | 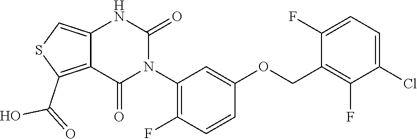 [SMILES: O=C(O)c1scc2[nH]c(=O)n(-c3cc(OCc4c(F)ccc(Cl)c4F)ccc3F)c(=O)c12] | (DMSO-d6) 5.14 (2H, s), 7.15-7.35 (3H, m), 7.35-7.45 (2H, m), 7.7-7.8 (1H, m), 12.02 (1H, s), 14.39 (1H, s) |
| 328 | 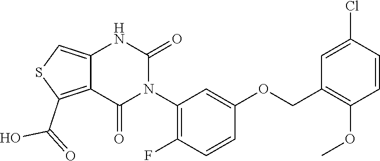 [SMILES: COc1ccc(Cl)cc1COc1ccc(F)c(-n2c(=O)[nH]c3csc(C(=O)O)c3c2=O)c1] | (DMSO-d6) 3.83 (3H, s), 5.01 (2H, s), 7.09 (1H, d, J = 8.8 Hz), 7.15-7.2 (1H, m), 7.25-7.35 (1H, m), 7.3-7.45 (3H, m), 7.48 (1H, d, J = 2.6 Hz), 12.02 (1H, s), 14.43 (1H, s) |
| 329 | 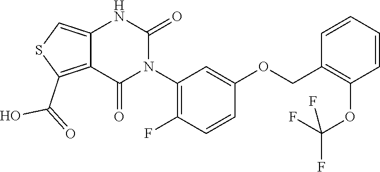 [SMILES: O=C(O)c1scc2[nH]c(=O)n(-c3cc(OCc4ccccc4OC(F)(F)F)ccc3F)c(=O)c12] | (DMSO-d6) 5.12 (2H, s), 7.15-7.25 (1H, m), 7.27 (1H, dd, J = 6.0 Hz, 3.2 Hz), 7.3-7.4 (2H, m), 7.4-7.5 (2H, m), 7.5-7.6 (1H, m), 7.7 (1H, dd, J = 7.4 Hz, 1.6 Hz), 12.01 (1H, s), 14.41 (1H, s) |
| 330 | 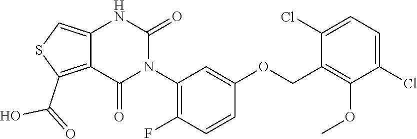 [SMILES: COc1c(Cl)ccc(Cl)c1COc1ccc(F)c(-n2c(=O)[nH]c3csc(C(=O)O)c3c2=O)c1] | (DMSO-d6) 3.85 (3H, s), 5.1 (2H, s), 7.2-7.35 (2H, m), 7.35-7.45 (3H, m), 7.63 (1H, d, J = 8.7 Hz), 12.01 (1H, s), 14.38 (1H, s) |
| 331 | 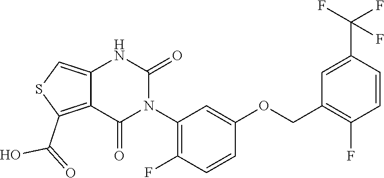 [SMILES: O=C(O)c1scc2[nH]c(=O)n(-c3cc(OCc4cc(C(F)(F)F)ccc4F)ccc3F)c(=O)c12] | (DMSO-d6) 5.21 (2H, s), 7.2-7.35 (2H, m), 7.35-7.45 (2H, m), 7.53 (1H, t, J = 9.2 Hz), 7.8-7.9 (1H, m), 8.02 (1H, d, J = 6.1 Hz), 12.02 (1H, s), 14.4 (1H, s) |
| 332 | 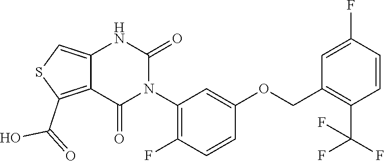 [SMILES: O=C(O)c1scc2[nH]c(=O)n(-c3cc(OCc4cc(F)ccc4C(F)(F)F)ccc3F)c(=O)c12] | (DMSO-d6) 5.21 (2H, s), 7.15-7.25 (1H, m), 7.3 (1H, dd, J = 5.8 Hz, 3.0 Hz), 7.35-7.5 (3H, m), 7.65-7.75 (1H, m), 7.89 (1H, dd, J = 8.7 Hz, 5.4 Hz), 12.01 (1H, s), 14.4 (1H, s) |
| 333 | 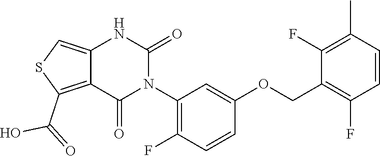 [SMILES: Cc1ccc(F)c(COc2ccc(F)c(-n3c(=O)[nH]c4csc(C(=O)O)c4c3=O)c2)c1F] | (DMSO-d6) 2.24 (3H, s), 5.08 (2H, s), 7.05-7.15 (1H, m), 7.15-7.3 (2H, m), 7.35-7.45 (3H, m), 12.02 (1H, s), 14.4 (1H, s) |
| 334 | 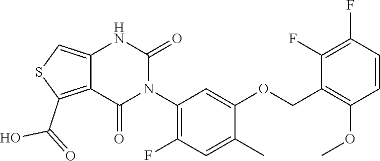 [SMILES: COc1ccc(F)c(F)c1COc1cc(-n2c(=O)[nH]c3csc(C(=O)O)c3c2=O)c(F)cc1C] | (DMSO-d6) 2.13 (3H, s), 3.82 (3H, s), 5.0 (2H, s), 6.85-6.95 (1H, m), 7.25 (1H, d, J = 10.0 Hz), 7.32 (1H, d, J = 6.4 Hz), 7.39 (1H, s), 7.4-7.55 (1H, m), 12.01 (1H, s), 14.47 (1H, s) |
| 335 | 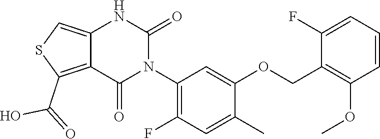 [SMILES: COc1cccc(F)c1COc1cc(-n2c(=O)[nH]c3csc(C(=O)O)c3c2=O)c(F)cc1C] | (DMSO-d6) 2.12 (3H, s), 3.84 (3H, s), 4.96 (2H, s), 6.8-7.0 (2H, m), 7.24 (1H, d, J = 10.2 Hz), 7.32 (1H, d, J = 6.3 Hz), 7.35-7.5 (2H, m), 12.04 (1H, s), 14.48 (1H, s) |
| 336 | 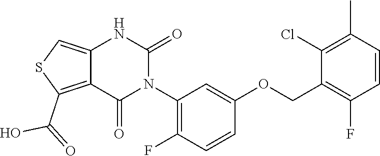 [SMILES: Cc1ccc(F)c(COc2ccc(F)c(-n3c(=O)[nH]c4csc(C(=O)O)c4c3=O)c2)c1Cl] | (DMSO-d6) 2.36 (3H, s), 5.1-5.15 (2H, m), 7.2-7.3 (3H, m), 7.35-7.45 (2H, m), 7.5 (1H, dd, J = 8.7 Hz, 6.2 Hz), 12.03 (1H, s), 14.4 (1H, s) |
| 337 | 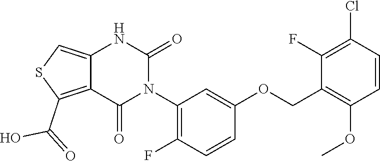 [SMILES: COc1ccc(Cl)c(F)c1COc1ccc(F)c(-n2c(=O)[nH]c3csc(C(=O)O)c3c2=O)c1] | (DMSO-d6) 3.86 (3H, s), 5.04 (2H, s), 7.0 (1H, d, J = 9.3 Hz), 7.15-7.25 (2H, m), 7.3-7.4 (2H, m), 7.55-7.65 (1H, m), 12.03 (1H, s), 14.41 (1H, s) |
| 338 | 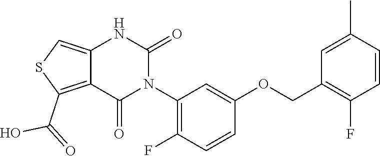 [SMILES: Cc1ccc(F)c(COc2ccc(F)c(-n3c(=O)[nH]c4csc(C(=O)O)c4c3=O)c2)c1] | (DMSO-d6) 2.3 (3H, s), 5.06 (2H, s), 7.1-7.45 (7H, m), 12.03 (1H, s), 14.43 (1H, s) |
| 339 | 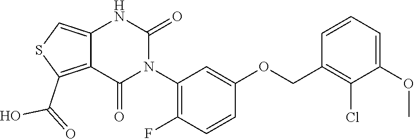 [SMILES: COc1cccc(COc2ccc(F)c(-n3c(=O)[nH]c4csc(C(=O)O)c4c3=O)c2)c1Cl] | (DMSO-d6) 3.88 (3H, s), 5.13 (2H, s), 7.15-7.25 (3H, m), 7.27 (1H, dd, J = 6.0 Hz, 3.2 Hz), 7.3-7.4 (3H, m), 12.02 (1H, s), 14.42 (1H, s) |
| 340 | 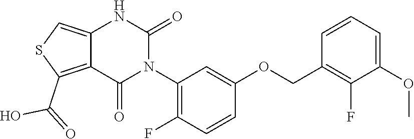 [SMILES: COc1cccc(COc2ccc(F)c(-n3c(=O)[nH]c4csc(C(=O)O)c4c3=O)c2)c1F] | (DMSO-d6) 3.85 (3H, s), 5.11 (2H, s), 7.05-7.3 (5H, m), 7.3-7.4 (2H, m), 12.02 (1H, s), 14.42 (1H, s) |
| 341 | 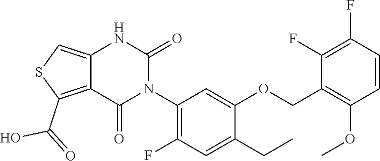 [SMILES: CCc1cc(F)c(-n2c(=O)[nH]c3csc(C(=O)O)c3c2=O)cc1OCc1c(OC)ccc(F)c1F] | (DMSO-d6) 1.1 (3H, t, J = 7.5 Hz), 2.45- 2.6 (2H, m), 3.82 (3H, s), 5.0 (2H, s), 6.9-6.85 (1H, m), 7.23 (1H, d, J = 10.3 Hz), 7.34 (1H, d, J = 6.3 Hz), 7.39 (1H, s), 7.4-7.55 (1H, m), 12.03 (1H, s), 14.47 (1H, s) |
| 342 | 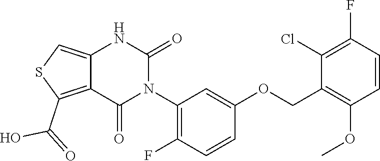 [SMILES: COc1ccc(F)c(Cl)c1COc1ccc(F)c(-n2c(=O)[nH]c3csc(C(=O)O)c3c2=O)c1] | (DMSO-d6) 3.84 (3H, s), 5.1 (2H, s), 7.13 (1H, dd, J = 9.2 Hz, 4.0 Hz), 7.15- 7.3 (2H, m), 7.3-7.4 (2H, m), 7.45-7.55 (1H, m), 12.03 (1H, s) |
| 343 | 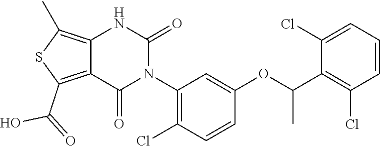 [SMILES: Cc1sc(C(=O)O)c2c(=O)n(-c3cc(OC(C)c4c(Cl)cccc4Cl)ccc3Cl)c(=O)[nH]c12] | (DMSO-d6) 1.73 (3H, d, J = 6.7 Hz), 2.5-2.55 (3H, m), 6.07 (1H, q, J = 6.7 Hz), 6.85-6.95 (1H, m), 7.15-7.2 (1H, m), 7.3-7.4 (1H, m), 7.45-7.55 (3H, m), 11.89 (1H, s), 14.37 (1H, s) |
| 344 | 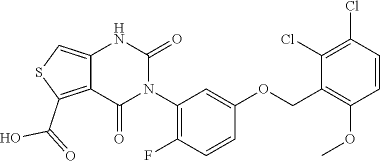 [SMILES: COc1ccc(Cl)c(Cl)c1COc1ccc(F)c(-n2c(=O)[nH]c3csc(C(=O)O)c3c2=O)c1] | (DMSO-d6) 3.86 (3H, s), 5.12 (2H, s), 7.1-7.3 (3H, m), 7.3-7.4 (2H, m), 7.69 (1H, d, J = 8.7 Hz), 12.03 (1H, s), 14.41 (1H, s) |
| 345 | 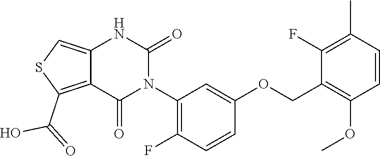 [SMILES: COc1ccc(C)c(F)c1COc1ccc(F)c(-n2c(=O)[nH]c3csc(C(=O)O)c3c2=O)c1] | (DMSO-d6) 2.15-2.2 (3H, m), 3.81 (3H, s), 5.0 (2H, s), 6.84 (1H, d, J = 8.5 Hz), 7.15-7.25 (2H, m), 7.25-7.4 (3H, m), 12.03 (1H, s), 14.42 (1H, s) |
| 346 | 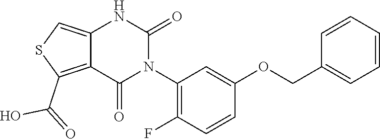 [SMILES: O=C(O)c1scc2[nH]c(=O)n(-c3cc(OCc4ccccc4)ccc3F)c(=O)c12] | (DMSO-d6) 5.08 (2H, s), 7.1-7.5 (9H, m), 12.01 (1H, s), 14.42 (1H, s) |
| 347 | 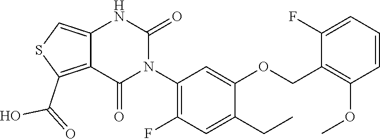 [SMILES: CCc1cc(F)c(-n2c(=O)[nH]c3csc(C(=O)O)c3c2=O)cc1OCc1c(F)cccc1OC] | (DMSO-d6) 1.1 (3H, t, J = 7.5 Hz), 2.45- 2.55 (2H, m), 3.84 (3H, s), 4.97 (2H, s), 6.85-7.0 (2H, m), 7.22 (1H, d, J = 10.2 Hz), 7.33 (1H, d, J = 6.4 Hz), 7.35-7.5 (2H, m), 12.02 (1H, s), 14.47 (1H, brs) |
| 348 | 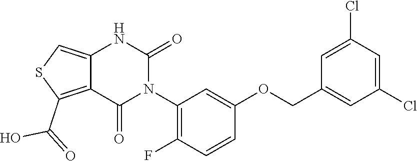 [SMILES: O=C(O)c1scc2[nH]c(=O)n(-c3cc(OCc4cc(Cl)cc(Cl)c4)ccc3F)c(=O)c12] | (DMSO-d6) 5.11 (2H, s), 7.1-7.3 (2H, m), 7.3-7.45 (2H, m), 7.5-7.65 (3H, m), 12.0 (1H, brs), 14.41 (1H, brs) |
| 349 | 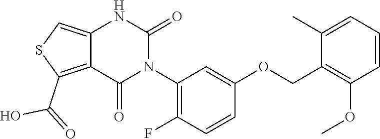 [SMILES: COc1cccc(C)c1COc1ccc(F)c(-n2c(=O)[nH]c3csc(C(=O)O)c3c2=O)c1] | (DMSO-d6) 2.33 (3H, s), 3.78 (3H, s), 5.04 (2H, s), 6.8-6.95 (2H, m), 7.1-7.3 (3H, m), 7.3-7.4 (2H, m), 12.01 (1H, s), 14.42 (1H, brs) |
| 350 | 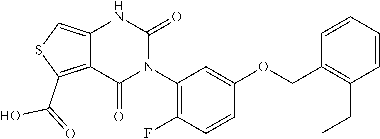 [SMILES: CCc1ccccc1COc1ccc(F)c(-n2c(=O)[nH]c3csc(C(=O)O)c3c2=O)c1] | (DMSO-d6) 1.19 (3H, t, J = 7.5 Hz), 2.68 (2H, q, J = 7.5 Hz), 5.07 (2H, s), 7.15-7.4 (7H, m), 7.4-7.5 (1H, m), 12.0 (1H, brs), 14.3-14.55 (1H, br) |
| 351 | 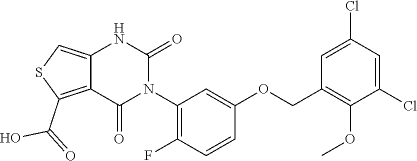 [SMILES: COc1c(Cl)cc(Cl)cc1COc1ccc(F)c(-n2c(=O)[nH]c3csc(C(=O)O)c3c2=O)c1] | (DMSO-d6) 3.84 (3H, s), 5.09 (2H, s), 7.2-7.25 (1H, m), 7.25-7.35 (1H, m), 7.35-7.45 (2H, m), 7.58 (1H, d, J = 2.7 Hz), 7.68 (1H, d, J = 2.7 Hz), 12.01 (1H, brs), 14.41 (1H, brs) |
| 352 | 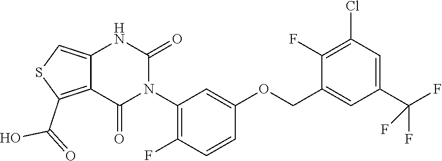 [SMILES: O=C(O)c1scc2[nH]c(=O)n(-c3cc(OCc4cc(C(F)(F)F)cc(Cl)c4F)ccc3F)c(=O)c12] | (DMSO-d6) 5.25 (2H, s), 7.2-7.35 (2H, m), 7.35-7.45 (2H, m), 7.95-8.05 (1H, m), 8.1-8.2 (1H, m), 12.01 (1H, brs), 14.4 (1H, brs) |
| 353 | 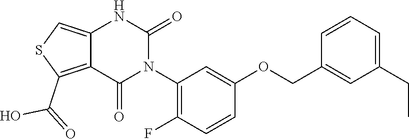 [SMILES: CCc1cccc(COc2ccc(F)c(-n3c(=O)[nH]c4csc(C(=O)O)c4c3=O)c2)c1] | (DMSO-d6) 1.19 (3H, t, J = 7.6 Hz), 2.62 (2H, q, J = 7.6 Hz), 5.05 (2H, s), 7.1-7.4 (8H, m), 12.01 (1H, brs), 14.43 (1H, brs) |
| 354 | 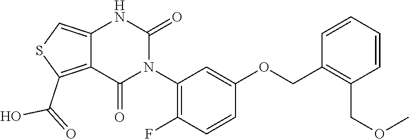 [SMILES: COCc1ccccc1COc1ccc(F)c(-n2c(=O)[nH]c3csc(C(=O)O)c3c2=O)c1] | (DMSO-d6) 3.3 (3H, s), 4.53 (2H, s), 5.13 (2H, s), 7.1-7.3 (2H, m), 7.3-7.45 (5H, m), 7.45-7.55 (1H, m), 12.01 (1H, brs), 14.42 (1H, brs) |
| 355 | 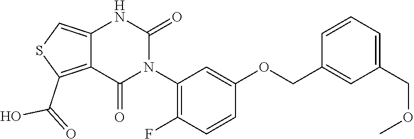 [SMILES: COCc1cccc(COc2ccc(F)c(-n3c(=O)[nH]c4csc(C(=O)O)c4c3=O)c2)c1] | (DMSO-d6) 3.29 (3H, s), 4.43 (2H, s), 5.09 (2H, s), 7.1-7.2 (1H, m), 7.2-7.45 (7H, m), 12.01 (1H, brs), 14.42 (1H, brs) |
| 356 | 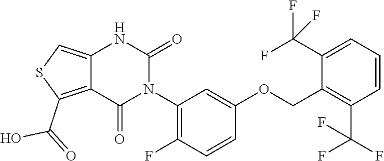 [SMILES: O=C(O)c1scc2[nH]c(=O)n(-c3cc(OCc4c(C(F)(F)F)cccc4C(F)(F)F)ccc3F)c(=O)c12] | (DMSO-d6) 5.19 (2H, s), 7.15-7.25 (1H, m), 7.25-7.3 (1H, m), 7.35-7.45 (2H, m), 7.92 (1H, t, J = 8.0 Hz), 8.21 (2H, d, J = 28.0 Hz), 12.01 (1H, s), 14.38 (1H, brs) |
| 357 | 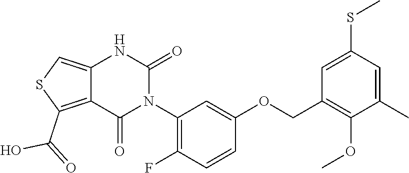 [SMILES: COc1c(C)cc(SC)cc1COc1ccc(F)c(-n2c(=O)[nH]c3csc(C(=O)O)c3c2=O)c1] | (DMSO-d6) 2.25 (3H, s), 2.44 (3H, s), 3.7 (3H, s), 5.02 (2H, s), 7.1-7.25 (3H, m), 7.25-7.3 (1H, m), 7.3-7.4 (2H, m), 12.01 (1H, brs), 14.3-14.55 (1H, br) |
| 358 | 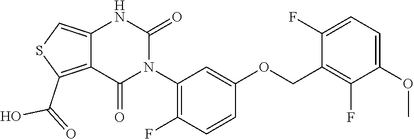 [SMILES: COc1ccc(F)c(COc2ccc(F)c(-n3c(=O)[nH]c4csc(C(=O)O)c4c3=O)c2)c1F] | (DMSO-d6) 3.85 (3H, s), 5.09 (2H, s), 7.05-7.35 (4H, m), 7.35-7.45 (2H, m), 12.02 (1H, brs), 14.39 (1H, brs) |
| 359 | 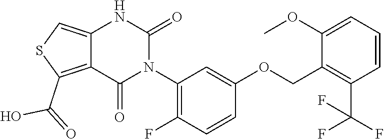 [SMILES: COc1cccc(C(F)(F)F)c1COc1ccc(F)c(-n2c(=O)[nH]c3csc(C(=O)O)c3c2=O)c1] | (DMSO-d6) 3.89 (3H, s), 5.07 (2H, s), 7.15-7.3 (2H, m), 7.3-7.5 (4H, m), 7.6- 7.7 (1H, m), 12.01 (1H, s), 14.4 (1H, brs) |
| 360 | 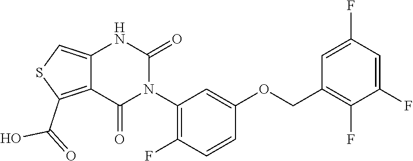 [SMILES: O=C(O)c1scc2[nH]c(=O)n(-c3cc(OCc4cc(F)cc(F)c4F)ccc3F)c(=O)c12] | (DMSO-d6) 5.18 (2H, s), 7.2-7.45 (5H, m), 7.5-7.65 (1H, m), 12.02 (1H, s), 14.4 (1H, brs) |
| 361 | 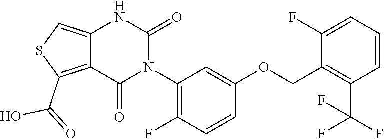 [SMILES: O=C(O)c1scc2[nH]c(=O)n(-c3cc(OCc4c(F)cccc4C(F)(F)F)ccc3F)c(=O)c12] | (DMSO-d6) 5.14 (2H, s), 7.15-7.3 (2H, m), 7.3-7.45 (2H, m), 7.6-7.8 (3H, m), 12.02 (1H, s), 14.39 (1H, s) |
| 362 | 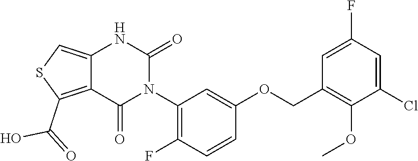 [SMILES: COc1c(Cl)cc(F)cc1COc1ccc(F)c(-n2c(=O)[nH]c3csc(C(=O)O)c3c2=O)c1] | (DMSO-d6) 3.82 (3H, s), 5.1 (2H, s), 7.15-7.45 (5H, m), 7.45-7.55 (1H, m), 12.01 (1H, s), 14.4 (1H, s) |
| 363 | 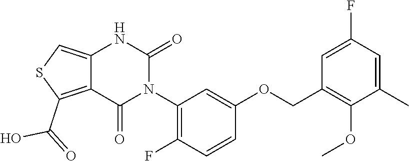 [SMILES: COc1c(C)cc(F)cc1COc1ccc(F)c(-n2c(=O)[nH]c3csc(C(=O)O)c3c2=O)c1] | (DMSO-d6) 2.27 (3H, s), 3.71 (3H, s), 5.05 (2H, s), 7.05-7.25 (3H, m), 7.25-7.3 (1H, m), 7.3-7.4 (2H, m), 12.02 (1H, s), 14.42 (1H, brs) |
| 364 | 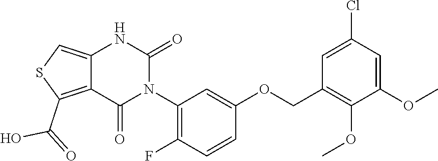 [SMILES: COc1cc(Cl)cc(COc2ccc(F)c(-n3c(=O)[nH]c4csc(C(=O)O)c4c3=O)c2)c1OC] | (DMSO-d6) 3.76 (3H, s), 3.85 (3H, s), 5.03 (2H, s), 7.05-7.3 (4H, m), 7.3-7.4 (2H, m), 12.02 (1H, brs), 14.42 (1H, brs) |
| 365 | 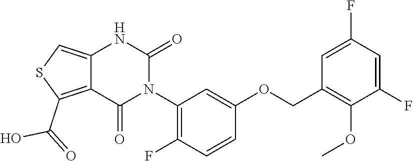 [SMILES: COc1c(F)cc(F)cc1COc1ccc(F)c(-n2c(=O)[nH]c3csc(C(=O)O)c3c2=O)c1] | (DMSO-d6) 3.8-3.9 (3H, m), 5.08 (2H, s), 7.15-7.25 (2H, m), 7.25-7.45 (4H, m), 12.02 (1H, brs), 14.41 (1H, brs) |
| 366 | 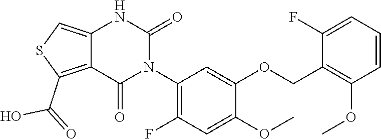 [SMILES: COc1cc(F)c(-n2c(=O)[nH]c3csc(C(=O)O)c3c2=O)cc1OCc1c(F)cccc1OC] | (DMSO-d6) 3.79 (3H, s), 3.83 (3H, s), 4.92 (2H, s), 6.8-7.0 (2H, m), 7.11 (1H, d, J = 11.2 Hz), 7.27 (1H, d, J = 27.6 Hz), 7.38 (1H, s), 7.4-7.5 (1H, m), 11.99 (1H, brs), 14.55 (1H, brs) |
| 367 | 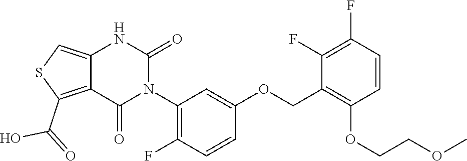 [SMILES: COCCOc1ccc(F)c(F)c1COc1ccc(F)c(-n2c(=O)[nH]c3csc(C(=O)O)c3c2=O)c1] | (DMSO-d6) 3.23 (3H, s), 3.55-3.65 (2H, m), 4.1-4.2 (2H, m), 5.09 (2H, s), 6.9-7.0 (1H, m), 7.15-7.3 (2H, m), 7.3-7.4 (2H, m), 7.4-7.5 (1H, m), 12.03 (1H, brs), 14.42 (1H, brs) |
| 368 | 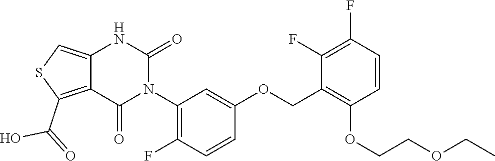 [SMILES: CCOCCOc1ccc(F)c(F)c1COc1ccc(F)c(-n2c(=O)[nH]c3csc(C(=O)O)c3c2=O)c1] | (DMSO-d6) 1.02 (3H, t, J = 6.9 Hz), 3.43 (2H, q, J = 6.9 Hz), 3.6-3.7 (2H, m), 4.1-4.2 (2H, m), 5.08 (2H, s), 6.9-7.0 (1H, m), 7.15-7.25 (2H, m), 7.3-7.4 (2H, m), 7.4-7.5 (1H, m), 12.02 (1H, s), 14.42 (1H, brs) |
| 369 | 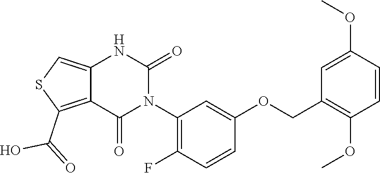 [SMILES: COc1ccc(OC)c(COc2ccc(F)c(-n3c(=O)[nH]c4csc(C(=O)O)c4c3=O)c2)c1] | (DMSO-d6) 3.71 (3H, s), 3.77 (3H, s), 4.99 (2H, s), 6.85-6.95 (1H, m), 6.95- 7.05 (2H, m), 7.1-7.2 (1H, m), 7.2-7.3 (1H, m), 7.3-7.4 (2H, m), 12.01 (1H, s), 14.44 (1H, brs) |
| 370 | 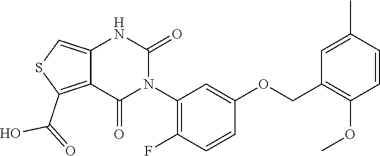 [SMILES: COc1ccc(C)cc1COc1ccc(F)c(-n2c(=O)[nH]c3csc(C(=O)O)c3c2=O)c1] | (DMSO-d6) 2.25 (3H, s), 3.78 (3H, s), 4.98 (2H, s), 6.94 (1H, d, J = 7.9 Hz), 7.1-7.2 (2H, m), 7.2-7.3 (2H, m), 7.3-7.4 (2H, m), 12.01 (1H, brs), 14.45 (1H, brs) |
| 371 | 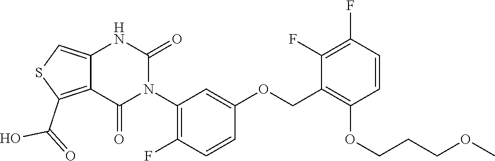 [SMILES: COCCCOc1ccc(F)c(F)c1COc1ccc(F)c(-n2c(=O)[nH]c3csc(C(=O)O)c3c2=O)c1] | (DMSO-d6) 1.85-1.95 (2H, m), 3.16 (3H, s), 3.39 (2H, t, J = 6.2 Hz), 4.08 (2H, t, J = 6.2 Hz), 5.06 (2H, s), 6.85-6.95 (1H, m), 7.15-7.3 (2H, m), 7.3-7.5 (3H, m), 12.03 (1H, s), 14.42 (1H, brs) |
| 372 | 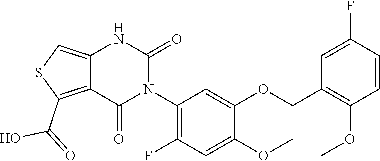 [SMILES: COc1ccc(F)cc1COc1cc(-n2c(=O)[nH]c3csc(C(=O)O)c3c2=O)c(F)cc1OC] | (DMSO-d6) 3.78 (3H, s), 3.85 (3H, s), 4.94 (2H, s), 7.0-7.1 (1H, m), 7.1-7.2 (2H, m), 7.2-7.35 (2H, m), 7.36 (1H, s), 11.96 (1H, brs), 14.56 (1H, brs) |
| 373 | 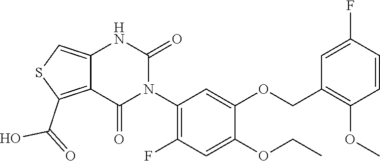 [SMILES: CCOc1cc(F)c(-n2c(=O)[nH]c3csc(C(=O)O)c3c2=O)cc1OCc1cc(F)ccc1OC] | (DMSO-d6) 1.36 (3H, t, J = 6.9 Hz), 3.78 (3H, s), 4.13 (2H, q, J = 6.9 Hz), 4.96 (2H, s), 7.0-7.1 (1H, m), 7.1-7.2 (2H, m), 7.2-7.35 (2H, m), 7.37 (1H, s), 11.97 (1H, brs), 14.56 (1H, brs) |
| 374 | 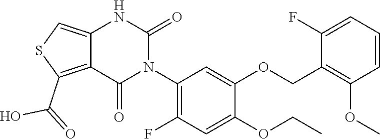 [SMILES: CCOc1cc(F)c(-n2c(=O)[nH]c3csc(C(=O)O)c3c2=O)cc1OCc1c(F)cccc1OC] | (DMSO-d6) 1.3 (3H, t, J = 7.0 Hz), 3.82 (3H, s), 4.08 (2H, q, J = 27.0 Hz), 4.94 (2H, s), 6.8-7.0 (2H, m), 7.1 (1H, d, J = 11.6 Hz), 7.26 (1H, d, J = 7.6 Hz), 7.35- 7.5 (2H, m), 11.99 (1H, s), 14.55 (1H, brs) |
| 375 | 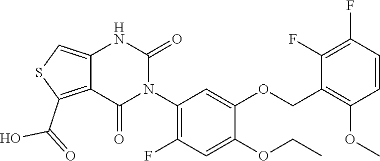 [SMILES: CCOc1cc(F)c(-n2c(=O)[nH]c3csc(C(=O)O)c3c2=O)cc1OCc1c(OC)ccc(F)c1F] | (DMSO-d6) 1.31 (3H, t, J = 7.0 Hz), 3.79 (3H, s), 4.09 (2H, q, J = 7.0 Hz), 4.95-5.05 (2H, m), 6.85-6.95 (1H, m), 7.12 (1H, d, J = 11.7 Hz), 7.23 (1H, d, J = 7.3 Hz), 7.38 (1H, s), 7.4-7.5 (1H, m), 11.99 (1H, s), 14.55 (1H, brs) |
| 376 | 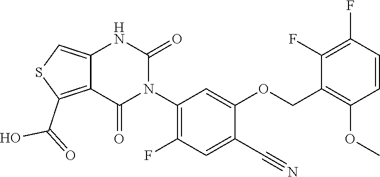 [SMILES: COc1ccc(F)c(F)c1COc1cc(-n2c(=O)[nH]c3csc(C(=O)O)c3c2=O)c(F)cc1C#N] | (DMSO-d6) 3.83 (3H, s), 5.18 (2H, s), 6.9-7.0 (1H, m), 7.38 (1H, s), 7.45-7.6 (1H, m), 7.7-7.85 (1H, m), 8.0-8.1 (1H, m), 12.08 (1H, s), 14.16 (1H, brs) |
| 377 | 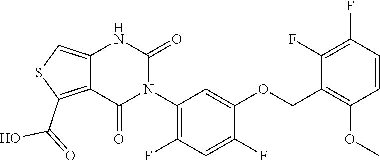 [SMILES: COc1ccc(F)c(F)c1COc1cc(-n2c(=O)[nH]c3csc(C(=O)O)c3c2=O)c(F)cc1F] | (DMSO-d6) 3.81 (3H, s), 5.07 (2H, s), 6.9-6.95 (1H, m), 7.39 (1H, s), 7.45- 7.55 (1H, m), 7.55-7.65 (2H, m), 12.06 (1H, s), 14.37 (1H, brs) |
| 378 | 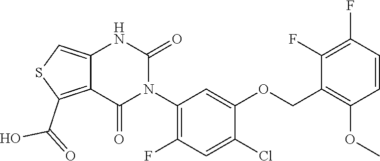 [SMILES: COc1ccc(F)c(F)c1COc1cc(-n2c(=O)[nH]c3csc(C(=O)O)c3c2=O)c(F)cc1Cl] | (DMSO-d6) 3.82 (3H, s), 5.08 (2H, s), 6.9-7.0 (1H, m), 7.39 (1H, s), 7.45-7.6 (1H, m), 7.61 (1H, d, J = 6.7 Hz), 7.73 (1H, d J = 9.1 Hz), 12.07 (1H, s), 14.33 (1H, brs) |
| 379 | 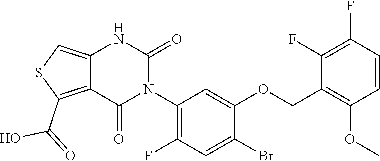 [SMILES: COc1ccc(F)c(F)c1COc1cc(-n2c(=O)[nH]c3csc(C(=O)O)c3c2=O)c(F)cc1Br] | (DMSO-d6) 3.82 (3H, s), 5.07 (2H, s), 6.9-7.0 (1H, m), 7.39 (1H, m), 7.45-7.55 (1H, m), 7.58 (1H, d, J = 6.5 Hz), 7.84 (1H, d, J = 8.8 Hz), 12.07 (1H, s), 14.33 (1H, s) |
| 380 | 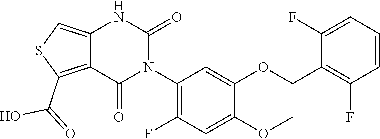 [SMILES: COc1cc(F)c(-n2c(=O)[nH]c3csc(C(=O)O)c3c2=O)cc1OCc1c(F)cccc1F] | (DMSO-d6) 3.81 (3H, s), 5.0 (2H, s), 7.1-7.25 (3H, m), 7.29 (1H, d, J = 7.4 Hz), 7.39 (1H, s), 7.45-7.6 (1H, m), 12.01 (1H, s), 14.54 (1H, s) |
| 381 | 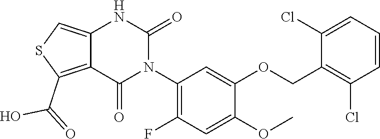 [SMILES: COc1cc(F)c(-n2c(=O)[nH]c3csc(C(=O)O)c3c2=O)cc1OCc1c(Cl)cccc1Cl] | (DMSO-d6) 3.82 (3H, s), 5.13 (2H, s), 7.15 (1H, d, J = 11.4 Hz), 7.33 (1H, d, J = 7.3 Hz), 7.39 (1H, s), 7.45-7.6 (3H, m), 12.01 (1H, s), 14.54 (1H, s) |
| 382 | 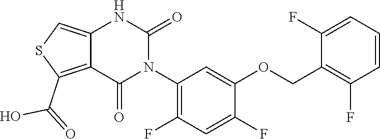 [SMILES: O=C(O)c1scc2[nH]c(=O)n(-c3cc(OCc4c(F)cccc4F)c(F)cc3F)c(=O)c12] | (DMSO-d6) 5.12 (2H, s), 7.15-7.25 (2H, m), 7.39 (1H, s), 7.5-7.7 (3H, m), 12.06 (1H, s), 14.36 (1H, brs) |
| 383 | 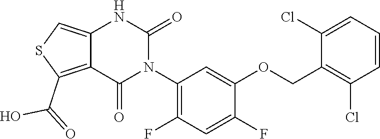 [SMILES: O=C(O)c1scc2[nH]c(=O)n(-c3cc(OCc4c(Cl)cccc4Cl)c(F)cc3F)c(=O)c12] | (DMSO-d6) 5.24 (2H, s), 7.39 (1H, s), 7.45-7.55 (1H, m), 7.55-7.45 (4H, m), 12.06 (1H, s), 14.36 (1H, brs) |
| 384 | 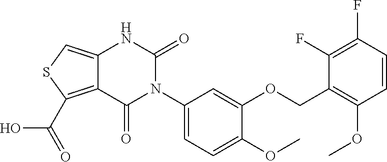 [SMILES: COc1ccc(-n2c(=O)[nH]c3csc(C(=O)O)c3c2=O)cc1OCc1c(OC)ccc(F)c1F] | (DMSO-d6) 3.78 (3H, s), 3.81 (3H, s), 4.96 (2H, s), 6.85-7.0 (2H, m), 7.07 (1H, d, J = 8.4 Hz), 7.18 (1H, d, J = 2.4 Hz), 7.37 (1H, s), 7.4-7.55 (1H, m), 11.87 (1H, s), 14.97 (1H, s) |
| 385 | 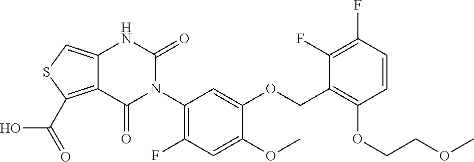 [SMILES: COCCOc1ccc(F)c(F)c1COc1cc(-n2c(=O)[nH]c3csc(C(=O)O)c3c2=O)c(F)cc1OC] | (DMSO-d6) 3.2 (3H, s), 3.5-3.6 (2H, m), 3.81 (3H, s), 4.05-4.15 (2H, m), 5.01 (2H, s); 6.85-6.95 (1H, m), 7.13 (1H, d, J = 11.2 Hz), 7.23 (1H, d, J = 7.0 Hz), 7.39 (1H, s), 7.4-7.5 (1H, m), 11.99 (1H, s), 14.52 (1H, s) |
| 386 | 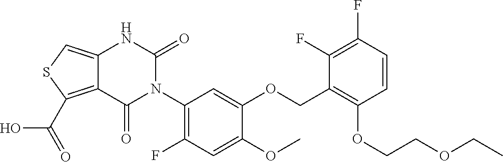 [SMILES: CCOCCOc1ccc(F)c(F)c1COc1cc(-n2c(=O)[nH]c3csc(C(=O)O)c3c2=O)c(F)cc1OC] | (DMSO-d6) 1.0 (3H, t, J = 7.0 Hz), 3.4 (2H, q, J = 7.0 Hz), 3.55-3.65 (2H, m), 3.81 (3H, s), 4.05-4.15 (2H, m), 5.0 (2H, s), 6.85-6.95 (1H, m), 7.13 (1H, d, J = 11.4 Hz), 7.23 (1H, d, J = 7.7 Hz), 7.38 (1H, s), 7.4-7.5 (1H, m), 11.99 (1H, s), 14.53 (1H, brs) |
| 387 | 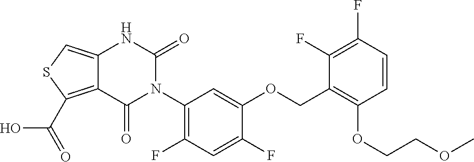 [SMILES: COCCOc1ccc(F)c(F)c1COc1cc(-n2c(=O)[nH]c3csc(C(=O)O)c3c2=O)c(F)cc1F] | (DMSO-d6) 3.19 (3H, s), 3.5-3.65 (2H, m), 4.05-4.2 (2H, m), 5.13 (2H, s), 6.9- 7.0 (1H, m), 7.38 (1H, s), 7.4-7.65 (3H, m), 12.03 (1H, s), 14.34 (1H, brs) |
| 388 | 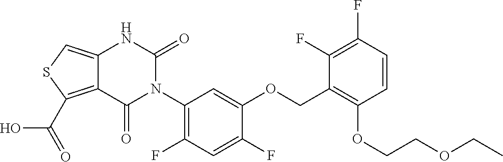 [SMILES: CCOCCOc1ccc(F)c(F)c1COc1cc(-n2c(=O)[nH]c3csc(C(=O)O)c3c2=O)c(F)cc1F] | (DMSO-d6) 0.99 (3H, t, J = 7.0 Hz), 3.4 (2H, q, J = 7.0 Hz), 3.55-3.7 (2H, m), 4.05-4.2 (2H, m), 5.11 (2H, s), 6.9-7.0 (1H, m), 7.38 (1H, s), 7.4-7.65 (3H, m), 12.03 (1H, s), 14.35 (1H, brs) |
| 389 | 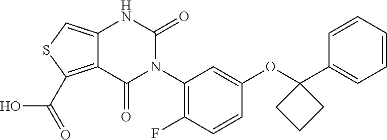 [SMILES: O=C(O)c1scc2[nH]c(=O)n(-c3cc(OC4(c5ccccc5)CCC4)ccc3F)c(=O)c12] | (DMSO-d6) 1.75-1.9 (1H, m), 1.9-2.05 (1H, m), 2.5-2.7 (4H, m), 6.55-6.6 (1H, m), 6.9-6.95 (1H, m), 7.1-7.2 (1H, m), 7.25-7.45 (4H, m), 7.45-7.55 (2H, m), 11.96 (1H, s), 14.41 (1H, s) |
| 390 | 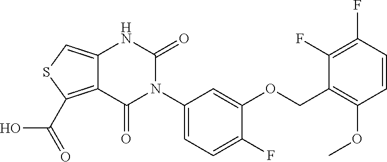 [SMILES: COc1ccc(F)c(F)c1COc1cc(-n2c(=O)[nH]c3csc(C(=O)O)c3c2=O)ccc1F] | (DMSO-d6) 3.82 (3H, s), 5.08 (2H, s), 6.9-7.0 (1H, m), 7.0-7.1 (1H, m), 7.3- 7.4 (2H, m), 7.4-7.55 (2H, m), 11.91 (1H, brs), 14.82 (1 H, brs) |
| 391 | 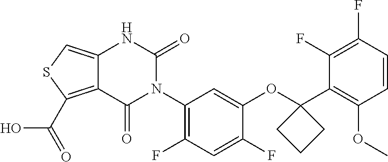 [SMILES: COc1ccc(F)c(F)c1C1(Oc2cc(-n3c(=O)[nH]c4csc(C(=O)O)c4c3=O)c(F)cc2F)CCC1] | (DMSO-d6) 1.8-1.95 (1H, m), 2.25-2.4 (1H, m), 2.6-2.8 (4H, m), 3.6 (3H, s), 6.65-6.75 (1H, m), 7.05-7.15 (1H, m), 7.25-7.35 (2H, m), 7.35-7.5 (1H, m), 11.7-12.2 (1H, br), 14.1-14.8 (1H, br) |
| 392 | 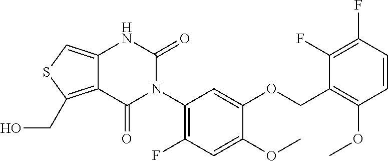 [SMILES: COc1cc(F)c(-n2c(=O)[nH]c3csc(CO)c3c2=O)cc1OCc1c(OC)ccc(F)c1F] | (DMSO-d6) 3.78 (3H, s), 3.81 (3H, s), 4.9-5.1 (4H, m), 5.95 (1H, t, J = 5.6 Hz), 6.71 (1H, s), 6.85-6.95 (1H, m), 7.05 (1H, d, J = 11.2 Hz), 7.17 (1H, d, J = 7.4 Hz), 7.4-7.55 (1H, m), 11.32 (1H, s) |
| 393 | 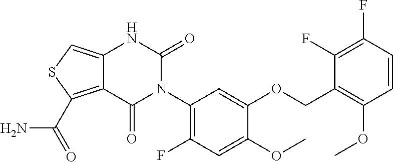 [SMILES: COc1cc(F)c(-n2c(=O)[nH]c3csc(C(N)=O)c3c2=O)cc1OCc1c(OC)ccc(F)c1F] | (DMSO-d6) 3.8 (3H, s), 3.81 (3H, s), 4.95 (2H, s), 6.85-6.95 (1H, m), 7.1 (1H, d, J = 11.5 Hz), 7.2-7.3 (2H, m), 7.4- 7.55 (1H, m), 8.05-8.15 (1H, m), 9.65- 9.75 (1H, m), 11.77 (1H, s) |
| 394 | 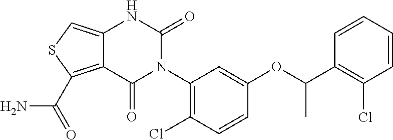 [SMILES: CC(Oc1ccc(Cl)c(-n2c(=O)[nH]c3csc(C(N)=O)c3c2=O)c1)c1ccccc1Cl] | (DMSO-d6) 1.58 (3H, d, J = 6.4 Hz), 5.65-5.75 (1H, m), 6.86-6.9 (1H, m), 7.2-7.25 (2H, m), 7.3-7.45 (2H, m), 7.45-7.55 (3H, m), 8.05-8.15 (1H, m), 9.6-9.7 (1H, m), 11.7-11.8 (1H, m) |
| 395 | 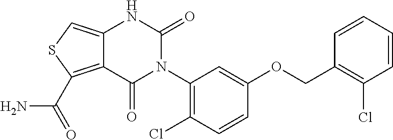 [SMILES: NC(=O)c1scc2[nH]c(=O)n(-c3cc(OCc4ccccc4Cl)ccc3Cl)c(=O)c12] | (DMSO-d6) 5.16 (2H, s), 7.19 (1H, dd, J = 8.9 Hz, 3.2 Hz), 7.24 (1H, s), 7.35- 7.45 (3H, m), 7.5-7.7 (3H, m), 8.11 (1H, d, J = 2.1 Hz), 9.65-9.7 (1H, m), 11.8 (1H, s) |
| 396 | 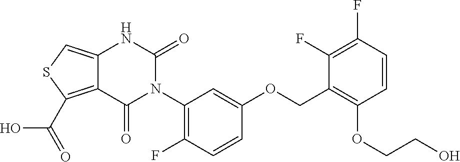 [SMILES: O=C(O)c1scc2[nH]c(=O)n(-c3cc(OCc4c(OCCO)ccc(F)c4F)ccc3F)c(=O)c12] | (DMSO-d6) 3.6-3.75 (2H, m), 4.04 (2H, t, J = 4.9 Hz), 4.8-4.95 (1H, m), 5.13 (2H, s), 6.9-6.95 (1H, m), 7.15-7.3 (2H, m), 7.3-7.4 (2H, m), 7.4-7.5 (1H, m), 12.01 (1H, s), 14.4 (1H, brs) |
| 397 | 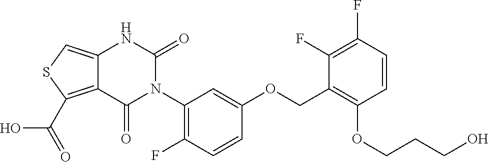 [SMILES: O=C(O)c1scc2[nH]c(=O)n(-c3cc(OCc4c(OCCCO)ccc(F)c4F)ccc3F)c(=O)c12] | (DMSO-d6) 1.75-1.9 (2H, m), 3.45-3.55 (2H, m), 4.1 (2H, t, J = 6.2 Hz), 4.9-5.0 (1H, m), 5.06 (2H, s), 6.85-6.95 (1H, m), 7.15-7.25 (2H, m), 7.3-7.4 (2H, m), 7.4- 7.5 (1H, m), 12.02 (1H, s), 14.4 (1H, brs) |
| 398 | 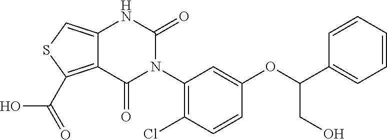 [SMILES: O=C(O)c1scc2[nH]c(=O)n(-c3cc(OC(CO)c4ccccc4)ccc3Cl)c(=O)c12] | (DMSO-d6) 3.55-3.65 (1H, m), 3.7-3.8 (1H, m), 5.15-5.25 (1H, m), 5.25-5.35 (1H, m), 7.0-7.05 (1H, m), 7.2-7.5 (8H, m), 11.95-12.05 (1H, m), 14.42 (1H, s) |
| 399 | 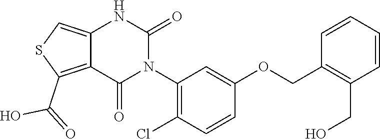 [SMILES: O=C(O)c1scc2[nH]c(=O)n(-c3cc(OCc4ccccc4CO)ccc3Cl)c(=O)c12] | (DMSO-d6) 4.61 (2H, s), 5.16 (2H, s), 5.21 (1H, brs), 7.15-7.5 (7H, m), 7.57 (1H, d, J = 8.6 Hz), 12.0 (1H, s) |
| 400 | 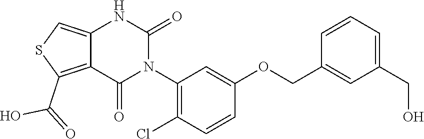 [SMILES: O=C(O)c1scc2[nH]c(=O)n(-c3cc(OCc4cccc(CO)c4)ccc3Cl)c(=O)c12] | (DMSO-d6) 4.51 (2H, J = 5.2 Hz), 5.1 (2H, s), 5.22 (1H, t, J = 5.2 Hz), 7.15-7.2 (1H, m), 7.25-7.45 (6H, m), 7.57 (1H, d, J = 8.6 Hz), 12.03 (1H, s), 14.44 (1H, s) |
| 401 | 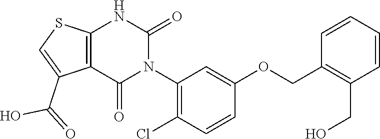 [SMILES: O=C(O)c1csc2[nH]c(=O)n(-c3cc(OCc4ccccc4CO)ccc3Cl)c(=O)c12] | (DMSO-d6) 4.61 (2H, s), 5.15 (2H, s), 7.15-7.5 (6H, m), 7.57 (1H, d, J = 9.2 Hz), 7.95 (1H, s), 13.95 (1H, s) |
| 402 | 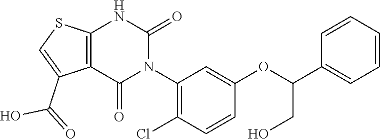 [SMILES: O=C(O)c1csc2[nH]c(=O)n(-c3cc(OC(CO)c4ccccc4)ccc3Cl)c(=O)c12] | (DMSO-d6) 3.55-3.65 (1H, m), 3.7-3.8 (1H, m), 5.0-5.4 (2H, m), 7.0-7.1 (1H, m), 7.15-7.5 (7H, m), 7.95 (1H, d, J = 5.9 Hz), 12.98 (1H, brs), 13.95 (1H, s) |
| 403 | 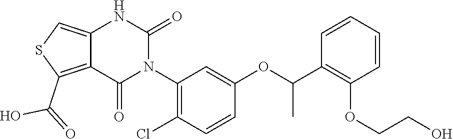 [SMILES: CC(Oc1ccc(Cl)c(-n2c(=O)[nH]c3csc(C(=O)O)c3c2=O)c1)c1ccccc1OCCO] | (DMSO-d6) 1.55 (3H, d, J = 6.3 Hz), 3.6-3.8 (2H, m), 3.95-4.15 (2H, m), 4.85-4.95 (1H, m), 5.75-5.9 (1H, m), 6.9-7.05 (3H, m), 7.15-7.5 (5H, m), 11.95-12.05 (1H, m), 14.43 (1H, s) |
| 404 | 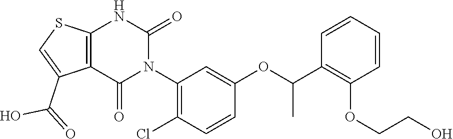 [SMILES: CC(Oc1ccc(Cl)c(-n2c(=O)[nH]c3scc(C(=O)O)c3c2=O)c1)c1ccccc1OCCO] | (DMSO-d6) 1.55 (3H, d, J = 6.3 Hz), 3.6-3.8 (2H, m), 3.95-4.15 (2H, m), 4.8- 5.0 (1H, br), 5.75-5.85 (1H, m), 6.9-7.05 (3H, m), 7.15-7.35 (3H, m), 7.43 (1H, d, J = 8.9 Hz), 7.9-8.0 (1H, m), 12.8-13.2 (1H, br), 13.99 (1H, s) |
| 405 | 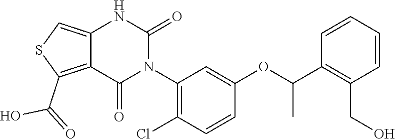 [SMILES: CC(Oc1ccc(Cl)c(-n2c(=O)[nH]c3csc(C(=O)O)c3c2=O)c1)c1ccccc1CO] | (DMSO-d6) 1.57 (3H, d, J = 6.2 Hz), 4.55-4.65 (1H, m), 4.65-4.75 (1H, m), 5.3-5.35 (1H, m), 5.7-5.8 (1H, m), 6.95- 7.05 (1H, m), 7.2-7.5 (7H, m), 11.95- 12.05 (1H, m), 14.42 (1H, s) |
| 406 | 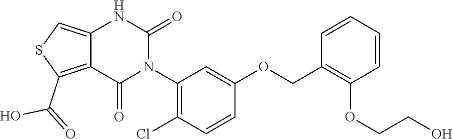 [SMILES: O=C(O)c1scc2[nH]c(=O)n(-c3cc(OCc4ccccc4OCCO)ccc3Cl)c(=O)c12] | (DMSO-d6) 3.65-3.75 (2H, m), 4.0-4.1 (2H, m), 4.87 (1H, brs), 5.12 (2H, s), 6.95-7.0 (1H, m), 7.05 (1H, d, J = 8.1 Hz), 7.19 (1H, dd, J = 9.0 Hz, 3.0 Hz), 7.25-7.45 (4H, m), 7.56 (1H, d, J = 9.0 Hz), 12.04 (1H, s), 14.46 (1H, s) |
| 407 | 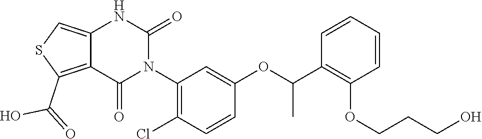 [SMILES: CC(Oc1ccc(Cl)c(-n2c(=O)[nH]c3csc(C(=O)O)c3c2=O)c1)c1ccccc1OCCCO] | (DMSO-d6) 1.54 (3H, d, J = 6.3 Hz), 1.85-1.95 (2H, m), 3.55-3.65 (2H, m), 4.05-4.2 (2H, m), 4.54 (1H, brs), 5.65- 5.75 (1H, m), 6.85-7.0 (2H, m), 7.0-7.1 (1H, m), 7.15-7.2 (1H, m), 7.2-7.35 (2H, m), 7.4 (1H, d, J = 4.6 Hz), 7.47 (1H, d, J = 9.0 Hz), 11.95-12.05 (1H, m), 14.42 (1H, s) |
| 408 | 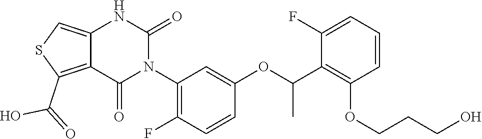 [SMILES: CC(Oc1ccc(F)c(-n2c(=O)[nH]c3csc(C(=O)O)c3c2=O)c1)c1c(F)cccc1OCCCO] | (DMSO-d6) 1.66 (3H, d, J = 6.4 Hz), 1.85-1.95 (2H, m), 3.5-3.6 (2H, m), 4.05-4.2 (2H, m), 5.75-5.85 (1H, m), 6.7-7.1 (4H, m), 7.2-7.4 (3H, 7), 11.95- 12.05 (1H, m), 14.35-14.45 (1H, m) |
| 409 | 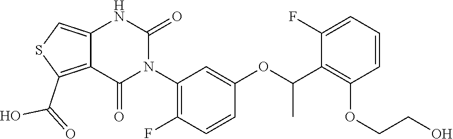 [SMILES: CC(Oc1ccc(F)c(-n2c(=O)[nH]c3csc(C(=O)O)c3c2=O)c1)c1c(F)cccc1OCCO] | (DMSO-d6) 1.66 (3H, d, J = 6.6 Hz), 3.55-3.8 (2H, m), 3.85-4.0 (1H, m), 4.05-4.2 (1H, m), 4.9-5.05 (1H, m), 5.9- 6.0 (1H, m), 6.7-6.9 (2H, m), 7.0-7.4 (5H, m), 11.95-12.05 (1H, m), 14.44 (1H, s) |
| 410 | 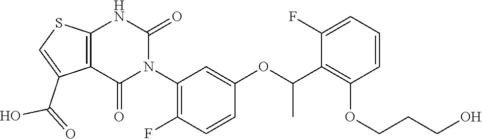 [SMILES: CC(Oc1ccc(F)c(-n2c(=O)[nH]c3scc(C(=O)O)c3c2=O)c1)c1c(F)cccc1OCCCO] | (DMSO-d6) 1.66 (3H, d, J = 6.4 Hz), 1.8-1.95 (2H, m), 3.5-3.6 (2H, m), 4.0- 4.2 (2H, m), 5.8 (1H, q, J = 6.4 Hz), 6.7- 6.8 (1H, m), 6.8-7.0 (2H, m), 7.0-7.05 (1H, m), 7.2-7.35 (2H, m), 7.9-7.95 (1H, m), 13.93 (1H, s) |
| 411 | 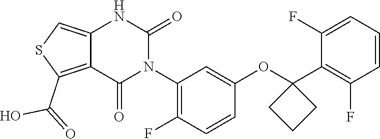 [SMILES: O=C(O)c1scc2[nH]c(=O)n(-c3cc(OC4(c5c(F)cccc5F)CCC4)ccc3F)c(=O)c12] | (DMSO-d6) 1.85-2.0 (1H, m), 2.2-2.35 (1H, m), 2.65-2.85 (4H, m), 6.75-6.85 (1H, m), 7.0-7.1 (3H, m), 7.24 (1H, t, J = 9.3 Hz), 7.3-7.45 (2H, m), 11.97 (1H, brs), 13.5-15.0 (1H, br) |
| 412 | 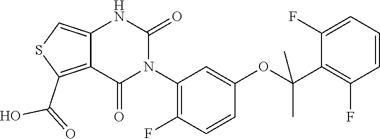 [SMILES: CC(C)(Oc1ccc(F)c(-n2c(=O)[nH]c3csc(C(=O)O)c3c2=O)c1)c1c(F)cccc1F] | (DMSO-d6) 1.820 (3H, s), 1.823 (3H, s), 6.75-6.8 (1H, m), 6.95-7.1 (3H, m), 7.15-7.25 (1H, m), 7.3-7.45 (2H, m), 11.96 (1H, brs), 14.43 (1H, brs) |
| 413 | 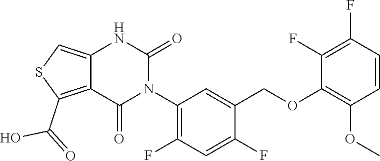 [SMILES: COc1ccc(F)c(F)c1OCc1cc(-n2c(=O)[nH]c3csc(C(=O)O)c3c2=O)c(F)cc1F] | (DMSO-d6) 3.79 (3H, s), 5.05-5.2 (2H, m), 6.8-6.9 (1H, m), 7.05-7.2 (1H, m), 7.37 (1H, s), 7.5-7.6 (1H, m), 7.65-7.75 (1H, m), 12.02 (1H, s), 14.35 (1H, s) |
| 414 | 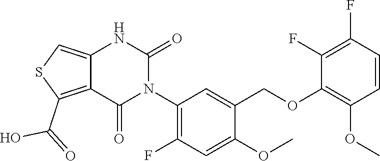 [SMILES: COc1cc(F)c(-n2c(=O)[nH]c3csc(C(=O)O)c3c2=O)cc1COc1c(OC)ccc(F)c1F] | (DMSO-d6) 3.79 (3H, s), 3.85 (3H, s), 5.0-5.1 (2H, m), 6.8-6.9 (1H, m), 7.05- 7.2 (2H, m), 7.38 (1H, s), 7.51 (1H, d, J = 8.5 Hz), 11.98 (1H, s), 14.54 (1H, brs) |
| 415 | 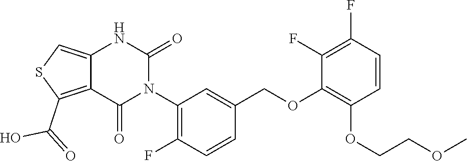 [SMILES: COCCOc1ccc(F)c(F)c1OCc1ccc(F)c(-n2c(=O)[nH]c3csc(C(=O)O)c3c2=O)c1] | (DMSO-d6) 3.27 (3H, s), 3.6-3.7 (2H, m), 4.1-4.2 (2H, m), 5.13 (2H, s), 6.85- 6.95 (1H, m), 7.05-7.2 (1H, m), 7.39 (1H, s), 7.4-7.5 (1H, m), 7.6-7.7 (2H, m), 12.05 (1H, s), 14.42 (1H, s) |
| 416 | 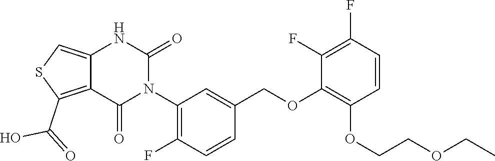 [SMILES: CCOCCOc1ccc(F)c(F)c1OCc1ccc(F)c(-n2c(=O)[nH]c3csc(C(=O)O)c3c2=O)c1] | (DMSO-d6) 1.05 (3H, t, J = 7.1 Hz), 3.46 (2H, q, J = 7.1 Hz), 3.65-3.75 (2H, m), 4.1-4.2 (2H, m), 5.14 (2H, s), 6.85- 6.95 (1H, m), 7.05-7.2 (1H, m), 7.38 (1H, s), 7.4-7.5 (1H, m), 7.55-7.7 (2H, m), 12.05 (1H, s), 14.41 (1H, s) |
| 417 | 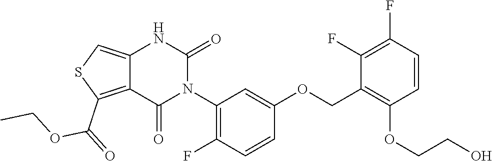 [SMILES: CCOC(=O)c1scc2[nH]c(=O)n(-c3cc(OCc4c(OCCO)ccc(F)c4F)ccc3F)c(=O)c12] | (DMSO-d6) 1.27 (3H, t, J = 7.1 Hz), 3.68 (2H, t, J = 4.8 Hz), 4.05 (2H, t, J = 4.8 Hz), 4.3 (2H, q, J = 7.1 Hz), 5.12 (2H, s), 6.85-6.95 (1H, m), 7.1-7.25 (3H, m), 7.25-7.35 (1H, m), 7.4-7.5 (1H, m), 11.63 (1H, s) |
| 418 | 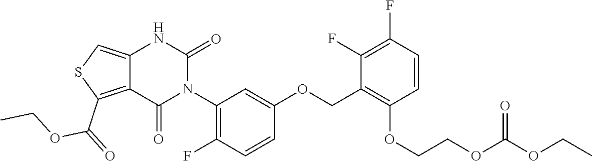 [SMILES: CCOC(=O)OCCOc1ccc(F)c(F)c1COc1ccc(F)c(-n2c(=O)[nH]c3csc(C(=O)OCC)c3c2=O)c1] | (DMSO-d6) 1.13 (3H, t, J = 7.1 Hz), 1.27 (3H, t, J = 7.1 Hz), 4.03 (2H, q, J = 7.0 Hz), 4.25-4.35 (4H, m), 4.35-4.45 (2H, m), 5.07 (2H, s), 6.9-7.0 (1H, m), 7.1-7.2 (3H, m), 7.25-7.35 (1H, m), 7.4- 7.55 (1H, m), 11.63 (1H, s) |
| 419 | 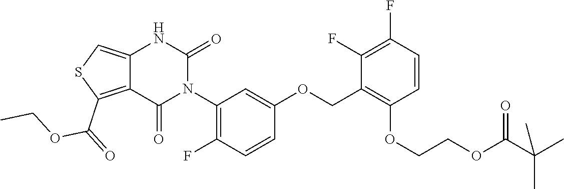 [SMILES: CCOC(=O)c1scc2[nH]c(=O)n(-c3cc(OCc4c(OCCOC(=O)C(C)(C)C)ccc(F)c4F)ccc3F)c(=O)c12] | (DMSO-d6) 1.04 (9H, s), 1.27 (3H, t, J = 7.1 Hz), 4.2-4.35 (6H, m), 5.05 (2H, s), 6.9-7.05 (1H, m), 7.05-7.2 (3H, m), 7.25-7.35 (1H, m), 7.4-7.55 (1H, m), 11.63 (1H, s) |
| 420 | 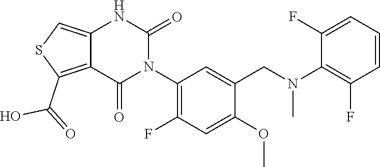 [SMILES: COc1cc(F)c(-n2c(=O)[nH]c3csc(C(=O)O)c3c2=O)cc1CN(C)c1c(F)cccc1F] | (DMSO-d6) 2.7-2.75 (3H, m), 3.71 (3H, s), 4.1-4.25 (2H, m), 6.95-7.15 (4H, m), 7.38 (1H, s), 7.41 (1H, d, J = 8.6 Hz), 11.95 (1H, s), 14.57 (1H, brs) |
| 421 | 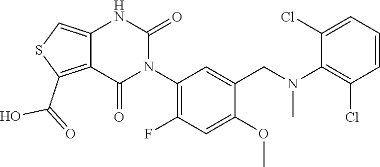 [SMILES: COc1cc(F)c(-n2c(=O)[nH]c3csc(C(=O)O)c3c2=O)cc1CN(C)c1c(Cl)cccc1Cl] | (DMSO-d6) 2.73 (3H, s), 3.74 (3H, s), 4.15-4.25 (2H, m), 7.08 (1H, d, J = 12.4 Hz), 7.15-7.2 (1H, m), 7.36 (1H, s), 7.4- 7.45 (2H, m), 7.56 (1H, d, J = 8.7 Hz), 11.96 (1H, brs), 14.61 (1H, brs) |
| 422 | 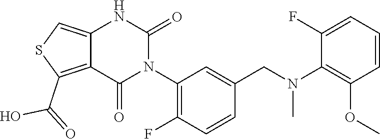 [SMILES: COc1cccc(F)c1N(C)Cc1ccc(F)c(-n2c(=O)[nH]c3csc(C(=O)O)c3c2=O)c1] | (DMSO-d6) 2.68 (3H, s), 3.81 (3H, s), 4.15 (2H, s), 6.7-6.85 (2H, m), 7.0-7.1 (1H, m), 7.3-7.4 (2H, m), 7.45-7.55 (2H, m), 11.98 (1H, s), 14.45 (1H, s) |
| 423 | 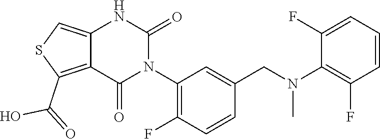 [SMILES: CN(Cc1ccc(F)c(-n2c(=O)[nH]c3csc(C(=O)O)c3c2=O)c1)c1c(F)cccc1F] | (DMSO-d6) 2.75 (3H, s), 4.22 (2H, s), 6.95-7.15 (3H, m), 7.35-7.45 (2H, m), 7.45-7.5 (2H, m), 12.02 (1H, s) |
| 424 | 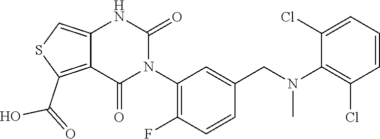 [SMILES: CN(Cc1ccc(F)c(-n2c(=O)[nH]c3csc(C(=O)O)c3c2=O)c1)c1c(Cl)cccc1Cl] | (DMSO-d6) 2.72 (3H, s), 4.3 (2H, s), 7.15-7.25 (1H, m), 7.35-7.5 (4H, m), 7.55-7.65 (2H, m), 12.03 (1H, s) |
| 425 | 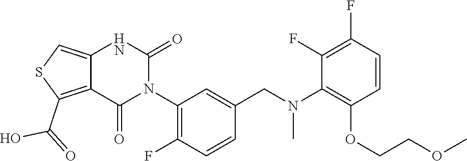 [SMILES: COCCOc1ccc(F)c(F)c1N(C)Cc1ccc(F)c(-n2c(=O)[nH]c3csc(C(=O)O)c3c2=O)c1] | (DMSO-d6) 2.7-2.75 (3H, m), 3.23 (3H, s), 3.6-3.7 (2H, m), 4.05-4.1 (2H, m), 4.22 (2H, s), 6.7-6.8 (1H, m), 7.0-7.1 (1H, m), 7.3-7.4 (2H, m), 7.45-7.55 (2H, m), 12.03 (1H, s), 14.46 (1H, brs) |
| 426 | 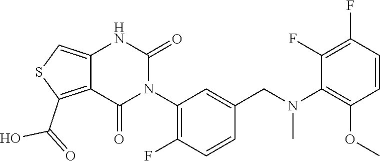 [SMILES: COc1ccc(F)c(F)c1N(C)Cc1ccc(F)c(-n2c(=O)[nH]c3csc(C(=O)O)c3c2=O)c1] | (DMSO-d6) 2.7-2.75 (3H, m), 3.79 (3H, s), 4.2 (2H, s), 6.7-6.8 (1H, m), 7.0-7.5 (1H, m), 7.3-7.4 (2H, m), 7.45-7.55 (2H, m), 12.02 (1H, s), 14.47 (1H, brs) |
| 427 | 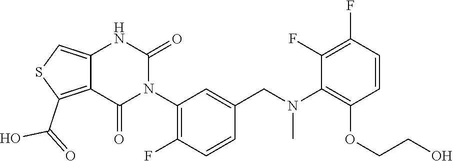 [SMILES: CN(Cc1ccc(F)c(-n2c(=O)[nH]c3csc(C(=O)O)c3c2=O)c1)c1c(OCCO)ccc(F)c1F] | (DMSO-d6) 3.7-3.8 (2H, m), 4.04 (2H, t, J = 4.6 Hz), 4.85-4.95 (1H, m), 5.17 (2H, s), 6.85-6.95 (1H, m), 7.05-7.15 (1H, m), 7.39 (1H, s), 7.4-7.5 (1H, m), 7.6-7.7 (2H, m), 12.05 (1H, s), 14.44 (1H, s) |
| 428 | 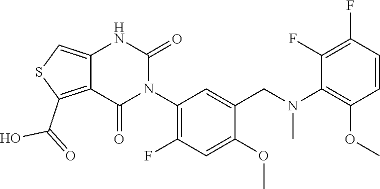 [SMILES: COc1cc(F)c(-n2c(=O)[nH]c3csc(C(=O)O)c3c2=O)cc1CN(C)c1c(OC)ccc(F)c1F] | (DMSO-d6) 2.7-2.75 (3H, m), 3.7-3.8 (6H, m), 4.1-4.2 (2H, m), 6.7-6.75 (1H, m), 6.95-7.1 (2H, m), 7.37 (1H, s), 7.41 (1H, d, J = 8.8 Hz), 11.95 (1H, s), 14.57 (1H, s) |

For example, the GnRH antagonist may be 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid, or a pharmaceutically acceptable salt thereof. The salt may be, for instance, the choline salt thereof, represented by formula (VIa), below.

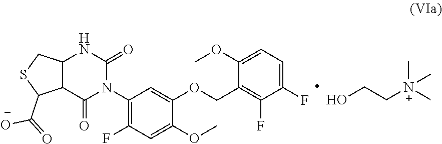

Compound (VI) and pharmaceutically acceptable salts thereof, such as the choline salt thereof (compound (VIa)), can be synthesized, for example, using the methodology described in WO 2014/042176, the disclosure of which is incorporated herein by reference in its entirety. An exemplary synthetic scheme that may be used for the preparation of compound (VI) and the choline salt thereof is shown in Scheme 1, below.

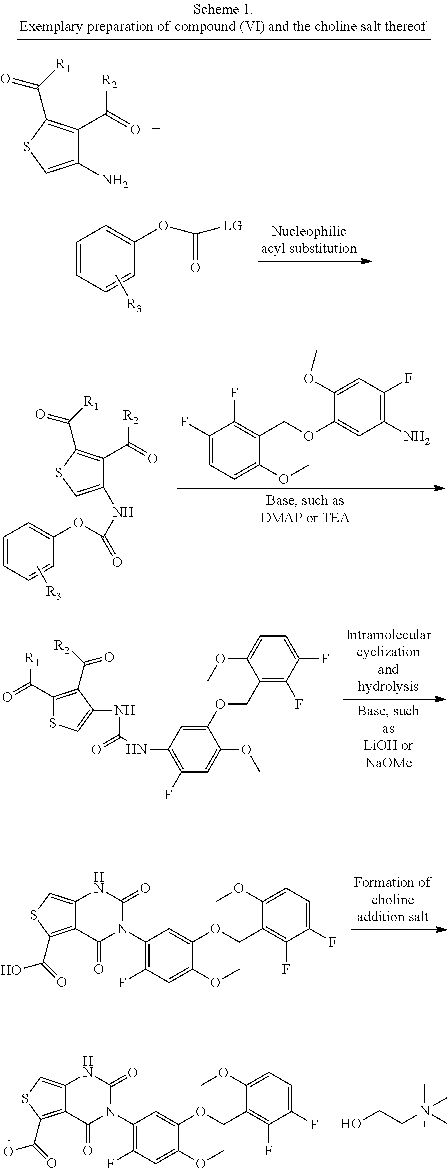

wherein R₁ and R₂ are each independently C₁₋₆ alkoxy groups; LG is a nucleofugal leaving group, such as chlorine or bromine, among others; R₃ represents an optional substituent, such as halogen, acyl group, C₁₋₆ alkyl group, or a nitro substituent; DMAP denotes N-dimethylaminopyridine; and TEA denotes trimethylamine.

Crystalline compound (VIa) has been characterized spectroscopically, for instance, in U.S. Pat. No. 9,169,266, the disclosure of which is incorporated herein by reference in its entirety. The foregoing crystalline form has been shown to exhibit characteristic X-ray powder diffraction peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ. Additionally, this crystalline form exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm. This crystalline form further exhibits ¹⁹F solid-state NMR peaks centered at about −151.8 ppm, −145.2 ppm, and −131.6 ppm.

Compound (VI), as well as pharmaceutically acceptable salts thereof, such as the choline salt thereof, exhibit a high affinity for human GnRH receptor (27.4 nM). Using the compositions and methods described herein, a patient that is presenting with or has been diagnosed as having an estrogen-dependent disease (e.g., uterine fibroids, endometriosis, such as rectovaginal endometriosis, and/or adenomyosis, among others described herein) may be administered a compound of formula (VI), or a pharmaceutically acceptable salt thereof, such as the choline salt thereof, to treat the disease or ameliorate one or more symptoms of the disease. Exemplary doses of compound (VI) and pharmaceutically acceptable salts thereof, such as the choline salt thereof, include doses of from 25 mg to 500 mg daily, such as doses of 100 mg per day and 200 mg per day. Additional dosing information is provided below.

### 3-Aminoalkyl pyrimidine-2,4(1H,3H)-diones

Additional GnRH antagonists that may be used in conjunction with the compositions and methods described herein include optionally substituted 3-aminoalkyl pyrimidine-2,4(1H,3H)-dione derivatives, such as compounds represented by formula (VII)

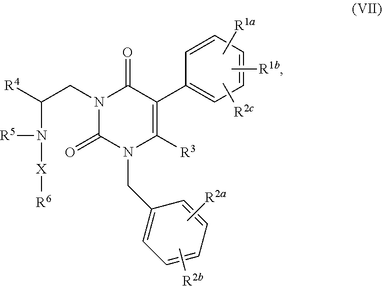

wherein R₁ₐ, R_{1b} and R_{1c} are the same or different and are each independently hydrogen, halogen, C₁₋₄alkyl, hydroxy or alkoxy, or R₁ₐ and R_{1b} taken together form —OCH₂O— or —OCH₂CH₂—;

R₂ₐ and R_{2b} are the same or different and are each independently hydrogen, halogen, trifluoromethyl, cyano or —SO₂CH₃;

R₃ is hydrogen or methyl;

R₄ is phenyl or C₃₋₇alkyl;

R₅ is hydrogen or C₁₋₄alkyl;

R₆ is —COOH or an acid isostere; and

X is C₁₋₆alkanediyl optionally substituted with from 1 to 3 C₁₋₆alkyl groups;

or a pharmaceutically acceptable salt thereof.

For example, the GnRH antagonist may be the conjugate acid of elagolix, which is represented by formula (VIII),

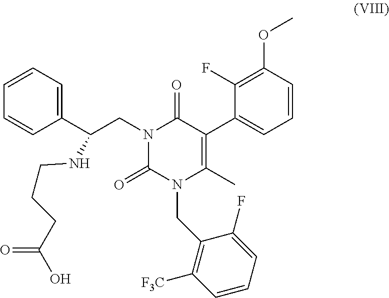 [SMILES: COc1cccc(-c2c(C)n(Cc3c(F)cccc3C(F)(F)F)c(=O)n(C[C@H](NCCCC(=O)O)c3ccccc3)c2=O)c1F]

or a pharmaceutically acceptable salt thereof. In some embodiments, the GnRH antagonist is the sodium salt of the compound represented by formula (VIII), which is represented by formula (IX), below.

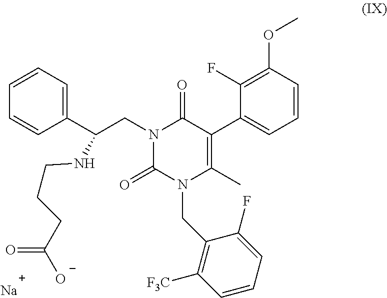 [SMILES: [Na+] ; COc1cccc(-c2c(C)n(Cc3c(F)cccc3C(F)(F)F)c(=O)n(C[C@H](NCCCC(=O)[O-])c3ccccc3)c2=O)c1F]

Compound (IX), also referred to as sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1 (2H)-yl]-1-phenylethyl}amino)butanoate, is known as elagolix. Other GnRH antagonists of this chemical class that may be used for the treatment of estrogen-dependent diseases in accordance with the compositions and methods of the disclosure include compounds described in U.S. Pat. No. 7,056,927, the contents of which are incorporated herein by reference. Exemplary GnRH antagonists of this chemical class that may be used for the treatment of estrogen-dependent diseases in accordance with the present disclosure include the compounds set forth in Table 2, below.

TABLE 2
Exemplary 3-Aminoalkyl pyrimidine-2,4(1H, 3H)-dione GnRH Antagonists
Useful for the Treatment of Estrogen-Dependent Diseases

| No. | Compound | Reported spectral properties |
|---|---|---|
| 1 | 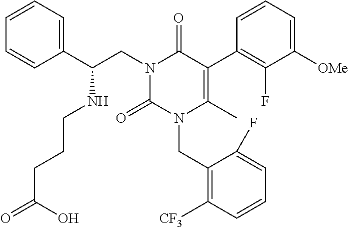 [SMILES: COc1cccc(-c2c(C)n(Cc3c(F)cccc3C(F)(F)F)c(=O)n(C[C@H](NCCCC(=O)O)c3ccccc3)c2=O)c1F] | HPLC-MS (Cl) m/z 632.2 (MH+), tR = 26.45 |
| 2 | 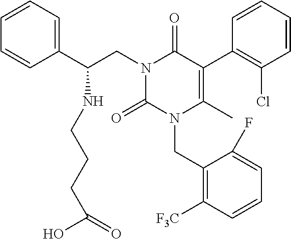 [SMILES: Cc1c(-c2ccccc2Cl)c(=O)n(C[C@H](NCCCC(=O)O)c2ccccc2)c(=O)n1Cc1c(F)cccc1C(F)(F)F] | MS (Cl) m/z 618.2 (MH+) tR = 1.005 |
| 3 | 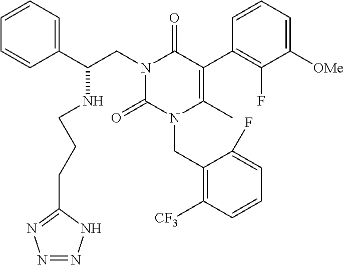 [SMILES: COc1cccc(-c2c(C)n(Cc3c(F)cccc3C(F)(F)F)c(=O)n(C[C@H](NCCCc3nnn[nH]3)c3ccccc3)c2=O)c1F] | HPLC-MS (Cl) m/z 656.2 (MH+), tR = 2.128 min |
| 4 | 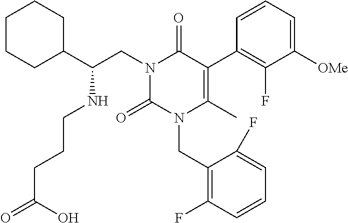 [SMILES: COc1cccc(-c2c(C)n(Cc3c(F)cccc3F)c(=O)n(C[C@H](NCCCC(=O)O)C3CCCCC3)c2=O)c1F] | MS (Cl) m/z 588.3 (MH+) |
| 5 | 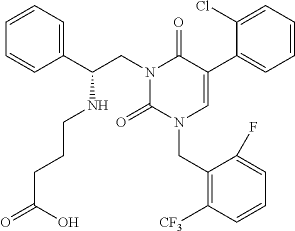 [SMILES: O=C(O)CCCN[C@@H](Cn1c(=O)c(-c2ccccc2Cl)cn(Cc2c(F)cccc2C(F)(F)F)c1=O)c1ccccc1] | HPLC-MS (Cl) m/z 604.1, 606.1 (MH+), tR = 2.511 |
| 6 | 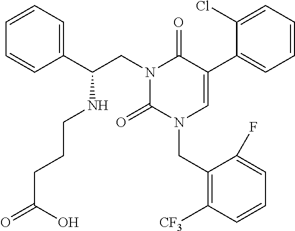 [SMILES: O=C(O)CCCN[C@@H](Cn1c(=O)c(-c2ccccc2Cl)cn(Cc2c(F)cccc2C(F)(F)F)c1=O)c1ccccc1] | HPLC-MS (Cl) m/z 604.1, 606.1 (MH+), tR = 26.98 |
| 7 | 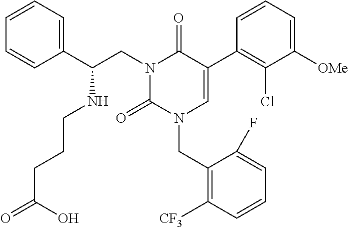 [SMILES: COc1cccc(-c2cn(Cc3c(F)cccc3C(F)(F)F)c(=O)n(C[C@H](NCCCC(=O)O)c3ccccc3)c2=O)c1Cl] | HPLC-MS (Cl) m/z 634.2, 636.2 (MH+), tR-24.925 |
| 8 | 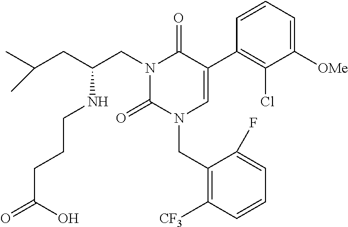 [SMILES: COc1cccc(-c2cn(Cc3c(F)cccc3C(F)(F)F)c(=O)n(C[C@@H](CC(C)C)NCCCC(=O)O)c2=O)c1Cl] | MS (Cl) m/z 614.1, 616.1 (MH+), tR = 6.550 min |
| 9 | 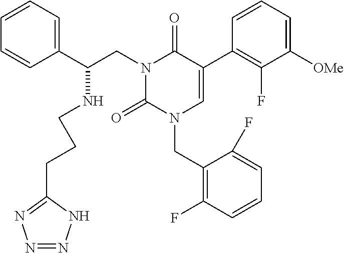 [SMILES: COc1cccc(-c2cn(Cc3c(F)cccc3F)c(=O)n(C[C@H](NCCCc3nnn[nH]3)c3ccccc3)c2=O)c1F] | HPLC-MS (Cl) m/z 592.3 (MH+), tR = 2.150 |
| 10 | 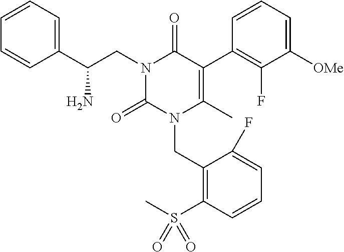 [SMILES: COc1cccc(-c2c(C)n(Cc3c(F)cccc3S(C)(=O)=O)c(=O)n(C[C@H](N)c3ccccc3)c2=O)c1F] | HPLC-MS (Cl) m/z = 556.2 (M + H+), tR = 2.354 |
| 11 | 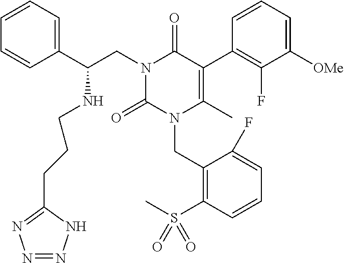 [SMILES: COc1cccc(-c2c(C)n(Cc3c(F)cccc3S(C)(=O)=O)c(=O)n(C[C@H](NCCCc3nnn[nH]3)c3ccccc3)c2=O)c1F] | |

### Thieno[2,3d]pyrimidines

Additional GnRH antagonists that may be used in conjunction with the compositions and methods described herein include optionally substituted thieno[2,3d]pyrimidine derivatives, such as compounds represented by formula (X)

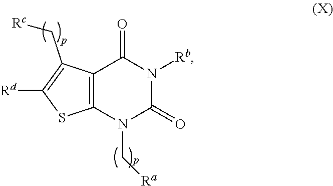

wherein R^{a} is a hydrogen atom, an optionally substituted aryl group (such as an aryl group that may have 1 to 5 substituents selected from halogen, nitro, cyano, amino, a carboxyl group that may be esterified or amidated, an alkylenedioxy, alkyl, alkoxy, alkylthio, alkylsulfinyl, and alkylsulfonyl), an optionally substituted cycloalkyl group, or an optionally substituted heterocyclic group;

R^{b} is an optionally substituted nitrogen-containing heterocyclic group;

R^{c} is an optionally substituted amino group;

R^{d} is an optionally substituted aryl group;

p is an integer from 0 to 3; and

q is an integer from 0 to 3;

or a pharmaceutically acceptable salt thereof.

In some embodiments, the GnRH antagonist is a thieno[2,3d]pyrimidine compound represented by formula (XI)

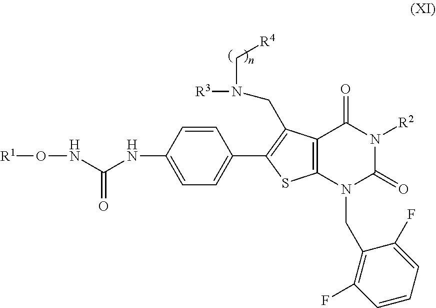

wherein R¹ is C₁₋₄alkyl;

R² is (1) a C₁₋₆alkyl which may have a substituent selected from the group consisting of (1′) a hydroxy group, (2′) a C₁₋₄alkoxy, (3′) a C₁₋₄alkoxy-carbonyl, (4′) a di-C₁₋₄alkyl-carbamoyl, (5′) a 5- to 7-membered nitrogen-containing heterocyclic group, (6′) a C₁₋₄alkyl-carbonyl and (7′) a halogen, (2) a C₃₋₈ cycloalkyl which may have (1′) a hydroxy group or (2′) a mono-C₁₋₄alkyl-carbonylamino, (3) a 5- to 7-membered nitrogen-containing heterocyclic group which may have a substituent selected from the group consisting of (1′) a halogen, (2′) a hydroxy group, (3′) a C₁₋₄alkyl and (4′) a C₁₋₄alkoxy, (4) a phenyl which may have a substituent selected from the group consisting of (1′) a halogen, (2′) a C₁₋₄alkoxy-C₁₋₄alkyl, (3′) a mono-C₁₋₄alkyl-carbamoyl-C₁₋₄alkyl, (4′) a C₁₋₄alkoxy and (5′) a mono-C₁₋₄alkylcarbamoyl-C₁₋₄alkoxy, or (5) a C₁₋₄alkoxy;

R³ is C₁₋₄alkyl;

R⁴ is (1) hydrogen, (2) C₁₋₄alkoxy, (3) C₆₋₁₀aryl, (4) N—C₁₋₄alkyl-N—C₁₋₄alkylsulfonylamino, (5) hydroxyl, or (6) a 5- to 7-membered nitrogen-containing heterocyclic group which may have a substituent selected from the group consisting of (1′) oxo, (2′) a C₁₋₄alkyl, (3′) a hydroxy-C₁₋₄alkyl, (4′) a C₁₋₄alkoxy-carbonyl, (5′) a mono-C₁₋₄alkyl-carbamoyl and (6′) a C₁₋₄alkylsulfonyl; and

n is an integer from 1 to 4;

optionally provided that when R² is a phenyl which may have a substituent, R⁴ is a 5- to 7-membered nitrogen-containing heterocyclic group which may have a substituent selected from the group consisting of (1) oxo, (2) hydroxy-C₁₋₄alkyl, (3) C₁₋₄alkoxy-carbonyl, (4) mono-C₁₋₄alkyl-carbamoyl and (5) C₁₋₄alkylsulfonyl;

or a pharmaceutically acceptable salt thereof. For example, the GnRH antagonist may be a compound represented by formula (XII), below.

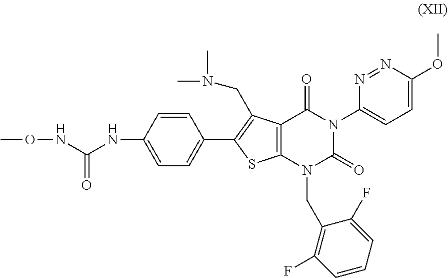

Compound (XII), also referred to as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, is known as relugolix. Other GnRH antagonists of this chemical class that may be used for the treatment of estrogen-dependent diseases in accordance with the compositions and methods of the disclosure include compounds described in U.S. Pat. No. 7,300,935, the contents of which are incorporated herein by reference. Exemplary GnRH antagonists of this chemical class that may be used for the treatment of estrogen-dependent diseases in accordance with the present disclosure include the compounds set forth in Table 3, below.

TABLE 3
Exemplary Thieno[2,3d]pyrimidine GnRH Antagonists Useful for the Treatment of Estrogen-
Dependent Diseases

| No. | Compound | Reported spectral properties |
|---|---|---|
| 1 | 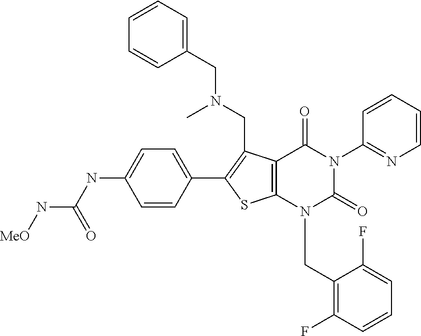 [SMILES: CONC(=O)Nc1ccc(-c2sc3c(c2CN(C)Cc2ccccc2)c(=O)n(-c2ccccn2)c(=O)n3Cc2c(F)cccc2F)cc1] | ¹H-NMR (CDCl₃) δ: 2.05 (3H, s), 3.56 (2H, s), 3.82 (3H, s), 3.89 (2H, s), 5.34 (2H, brs), 6.91 (2H, t, J = 8.0 Hz), 7.1- 7.45 (9H, m), 7.56 (2H, d, J = 8.8 Hz), 7.65 (1H, s), 7.75 (2H, d, J = 8.8 Hz), 7.91 (1H, dt, J = 2.0, 7.7 Hz), 8.7-8.75 (1H, m). |
| 2 | 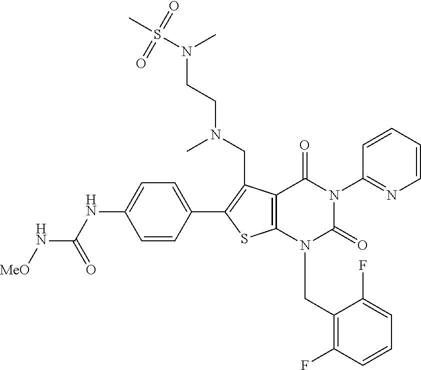 [SMILES: CONC(=O)Nc1ccc(-c2sc3c(c2CN(C)CCN(C)S(C)(=O)=O)c(=O)n(-c2ccccn2)c(=O)n3Cc2c(F)cccc2F)cc1] | ¹H-NMR (CDCl₃) δ: 2.11 (3H, s), 2.45-2.6 (2H, m), 2.70 (3H, s), 2.75 (3H, s), 3.1-3.25 (2H, m), 3.80 (2H, s), 3.83 (3H, s), 5.36 (2H, brs), 6.93 (2H, t, J = 8.2 Hz), 7.14 (1H, s), 7.2- 7.6 (7H, m), 7.65 (1H, s), 7.85- 7.95 (1H, m), 8.65-8.75 (1H, m). |
| 3 | 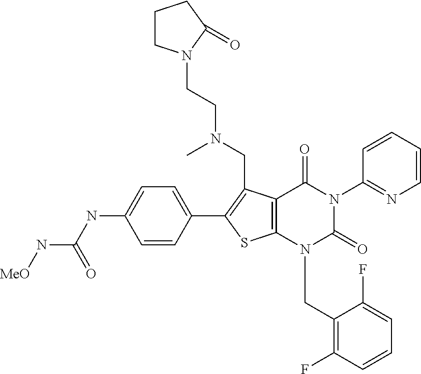 [SMILES: CONC(=O)Nc1ccc(-c2sc3c(c2CN(C)CCN2CCCC2=O)c(=O)n(-c2ccccn2)c(=O)n3Cc2c(F)cccc2F)cc1] | ¹H-NMR (CDCl₃) δ: 1.7-1.85 (2H, m), 2.17 (3H, s), 2.15-2.3 (2H, m), 2.5-2.6 (2H, m), 3.15 (2H, t, J = 7.0 Hz), 3.2-3.4 (2H, m), 3.76 (2H, s), 3.83 (3H, s), 5.36 (2H, brs), 6.93 (2H, t, J = 8.4 Hz), 7.16 (1H, s), 7.2- 7.7 (8H, m), 7.85-7.95 (1H, m), 8.65-8.75 (1H, m). |
| 4 | 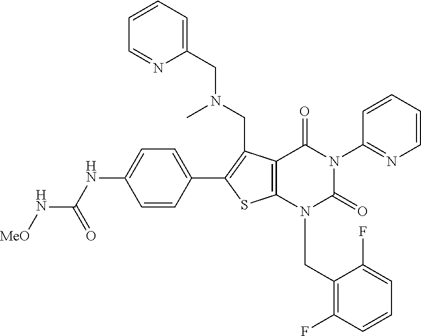 [SMILES: CONC(=O)Nc1ccc(-c2sc3c(c2CN(C)Cc2ccccn2)c(=O)n(-c2ccccn2)c(=O)n3Cc2c(F)cccc2F)cc1] | ¹H-NMR (CDCl₃) δ: 2.10 (3H, s), 3.70 (2H, s), 3.82 (3H, s), 3.96 (3H, s), 5.34 (2H, brs), 6.85-7.7 (14H, m), 7.85-7.95 (1H, m), 8.4-8.5 (1H, m), 8.65- 8.75 (1H, m). |
| 5 | 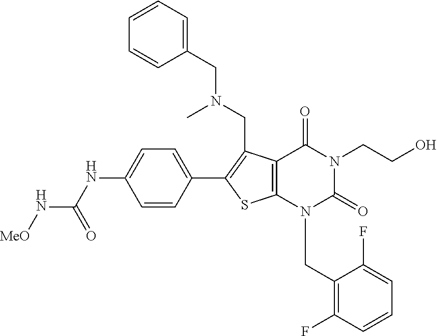 [SMILES: CONC(=O)Nc1ccc(-c2sc3c(c2CN(C)Cc2ccccc2)c(=O)n(CCO)c(=O)n3Cc2c(F)cccc2F)cc1] | ¹H-NMR (CDCl₃) δ: 2.04 (3H, s), 2.5-2.65 (1H, m), 3.58 (2H, s), 3.83 (3H, s), 3.91 (2H, s), 3.9-4.0 (2H, m), 4.37 (2H, t, J = 5.0 Hz), 5.34 (2H, s), 6.92 (2H, t, J = 8.2 Hz), 7.1-7.4 (7H, m), 7.54 (2H, d, J = 8.6 Hz), 7.66 (2H, d, J = 8.6 Hz), 7.6- 7.7 (1 H, m). |
| 6 | 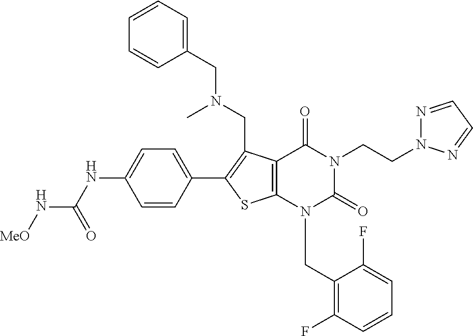 [SMILES: CONC(=O)Nc1ccc(-c2sc3c(c2CN(C)Cc2ccccc2)c(=O)n(CCn2nccn2)c(=O)n3Cc2c(F)cccc2F)cc1] | ¹H-NMR (CDCl₃) δ: 2.04 (3H, s), 3.51 (2H, s), 3.82 (3H, s), 3.86 (2H, s), 4.57 (2H, t, J = 6.2 Hz), 4.81 (2H, t, J = 6.2 Hz), 5.28 (2H, s), 6.91 (2H, t, J = 8.4 Hz), 7.15-7.35 (6H, m), 7.46 (2H, s), 7.53 (2H, d, J = 8.6 Hz), 7.62 (1H, s), 7.70 (2H, t, J = 8.6 Hz), 7.75 (1H, s). |
| 7 | 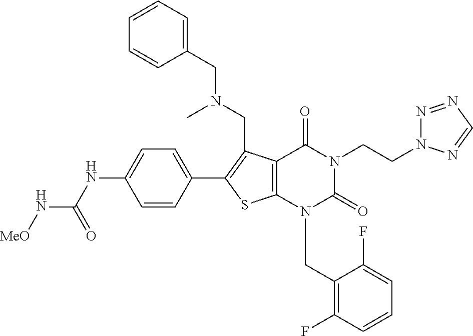 [SMILES: CONC(=O)Nc1ccc(-c2sc3c(c2CN(C)Cc2ccccc2)c(=O)n(CCn2ncnn2)c(=O)n3Cc2c(F)cccc2F)cc1] | 2-yl Form (1) ¹H-NMR (CDCl₃) δ: 2.02 (3H, s), 3.51 (2H, s), 3.83 (5H, s), 4.6-4.7 (2H, m), 5.0-5.1 (2H, m), 5.28 (2H, s), 6.92 (2H, t, J = 8.2 Hz), 7.12 (1H, s), 7.2- 7.75 (11H, m), 8.38 (1H, s). |
| | 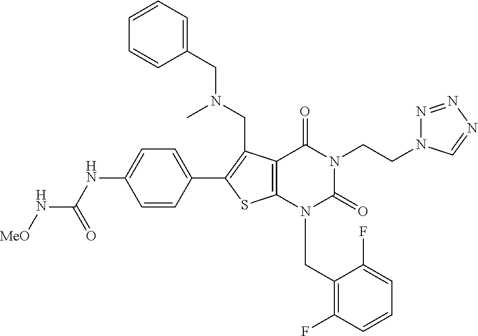 [SMILES: CONC(=O)Nc1ccc(-c2sc3c(c2CN(C)Cc2ccccc2)c(=O)n(CCn2cnnn2)c(=O)n3Cc2c(F)cccc2F)cc1] | 1-yl Form (2) ¹H-NMR (CDCl₃) δ: 2.01 (3H, s), 3.51 (2H, s), 3.83 (3H, s), 3.8-4.0 (2H, m), 4.6-4.7 (2H, m), 4.8-4.9 (2H, m), 5.30 (2H, s), 6.65-6.75 (1H, m), 6.85-7.0 (2H, m), 7.1-7.7 (11H, m), 8.68 (1H, s). |
| 8 | 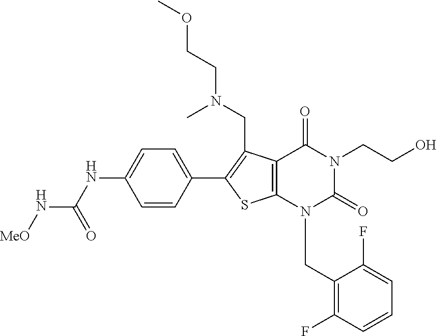 [SMILES: COCCN(C)Cc1c(-c2ccc(NC(=O)NOC)cc2)sc2c1c(=O)n(CCO)c(=O)n2Cc1c(F)cccc1F] | ¹H-NMR (CDCl₃) δ: 2.11 (3H, s), 2.65 (2H, t, J = 5.8 Hz), 3.30 (3H, s), 3.46 (2H, t, J = 5.8 Hz), 3.82 (5H, s), 3.9-4.0 (2H, m), 4.35 (2H, t, J = 5.2 Hz), 5.34 (2H, s), 6.92 (2H, t, J = 8.0 Hz), 7.14 (1H, s), 7.2-7.35 (1H, m), 7.5-7.65 (5H, m). |
| 9 | 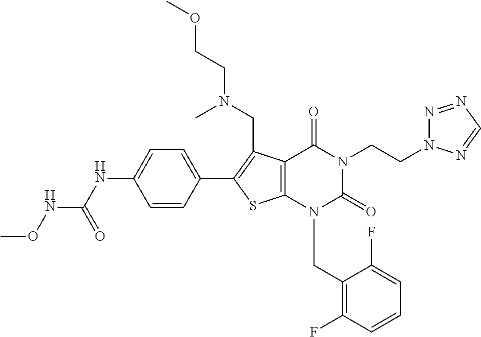 [SMILES: COCCN(C)Cc1c(-c2ccc(NC(=O)NOC)cc2)sc2c1c(=O)n(CCn1ncnn1)c(=O)n2Cc1c(F)cccc1F] | 2-yl Form (1) ¹H-NMR (CDCl₃) δ: 2.10 (3H, brs), 2.55-2.65 (2H, m), 3.31 (3H, s), 3.4-3.5 (2H, m), 3.76 (2H, s), 3.82 (3H, s), 4.62 (2H, t, J = 5.8 Hz), 5.02 (2H, t, J = 5.8 Hz), 5.27 (2H, s), 6.92 (2H, t, J = 8.2 Hz), 7.13 (1H, s), 7.25- 7.4 (1H, m), 7.5-7.65 (5H, m), 8.43 (1H, s). |
| | 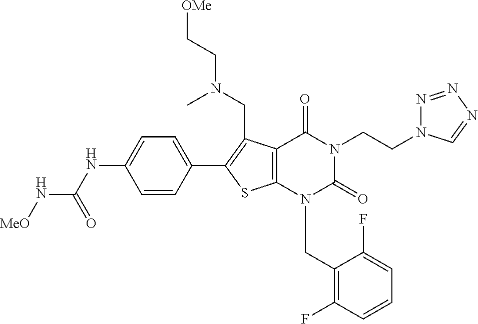 [SMILES: COCCN(C)Cc1c(-c2ccc(NC(=O)NOC)cc2)sc2c1c(=O)n(CCn1cnnn1)c(=O)n2Cc1c(F)cccc1F] | 1-yl Form (2) ¹H-NMR (CDCl₃) δ: 2.07 (3H, s), 2.55-2.65 (2H, m), 3.30 (3H, s), 3.4-3.5 (2H, m), 3.74 (2H, s), 3.82 (3H,s), 4.55-4.65 (2H, m), 4.8-4.9 (2H, m), 5.30 (2H, s), 6.93 (2H, t, J = 7.8 Hz), 7.10 (1H, s), 7.2-7.4 (1H, m), 7.5-7.65 (5H, m), 8.69 (1H,S). |
| 10 | 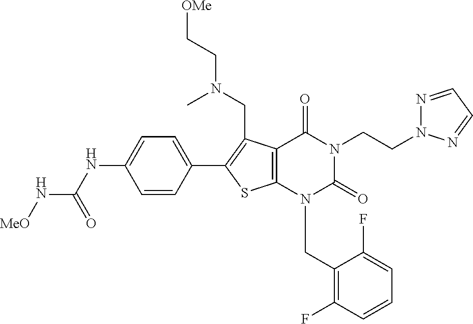 [SMILES: COCCN(C)Cc1c(-c2ccc(NC(=O)NOC)cc2)sc2c1c(=O)n(CCn1nccn1)c(=O)n2Cc1c(F)cccc1F] | ¹H-NMR (CDCl₃) δ: 2.11 (3H, s), 2.62 (2H, t, J = 5.8 Hz), 3.31 (3H, s), 3.44 (2H, t, J = 5.8 Hz), 3.78 (2H, s), 3.82 (3H, s), 4.55 (2H, t, J = 5.6 Hz), 4.79 (2H, t, J = 5.6 Hz), 5.27 (2H, s), 6.91 (2H, t, J = 8.2 Hz), 7.10 (1H, s), 7.2-7.4 (2H, m), 7.51 (2H, s), 7.45-7.65 (4H, m). |
| 11 | 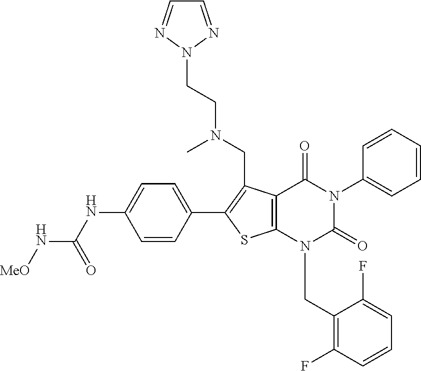 [SMILES: CONC(=O)Nc1ccc(-c2sc3c(c2CN(C)CCn2nccn2)c(=O)n(-c2ccccc2)c(=O)n3Cc2c(F)cccc2F)cc1] | 2-yl Form (1) ¹H-NMR (CDCl₃) δ: 2.23 (3H, s), 3.00 (2H, t, J = 6.3 Hz), 3.78 (2H, s), 3.82 (3H, s), 4.45 (2H, t, J = 6.3 Hz), 5.37 (2H, s), 6.92 (2H, t, J = 8.2 Hz), 6.85-6.95 (1H, m), 7.11 (1H, s), 7.2-7.6 (12H, m). |
| | 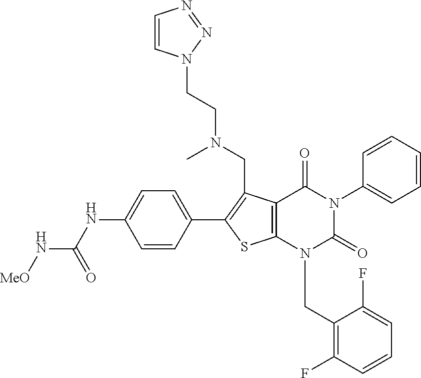 [SMILES: CONC(=O)Nc1ccc(-c2sc3c(c2CN(C)CCn2ccnn2)c(=O)n(-c2ccccc2)c(=O)n3Cc2c(F)cccc2F)cc1] | 1-yl Form (2) ¹H-NMR (CDCl₃) δ: 2.14 (3H, s), 2.82 (2H, t, J = 6.0 Hz), 3.80 (2H, s), 3.82 (3H, s), 4.39 (2H, t, J = 6.0 Hz), 5.37 (2H, s), 6.92 (2H, t, J = 8.2 Hz), 6.85-6.95 (1H, m), 7.14 (1H, s), 7.2-7.55 (11H, m), 7.63 (1H, s). |
| 12 | 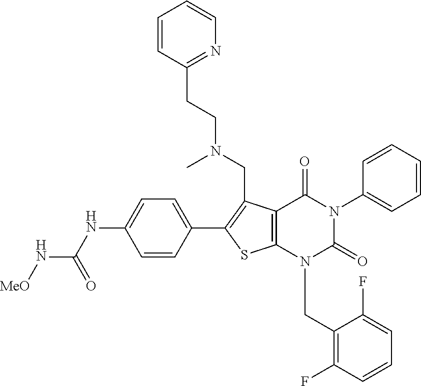 [SMILES: CONC(=O)Nc1ccc(-c2sc3c(c2CN(C)CCc2ccccn2)c(=O)n(-c2ccccc2)c(=O)n3Cc2c(F)cccc2F)cc1] | ¹H-NMR (CDCl₃) δ: 2.20 (3H, s), 2.85 (4H, s), 3.82 (5H, s), 5.37 (2H, s), 6.93 (2H, t, J = 8.2 Hz), 6.95-7.1 (2H, m),7.14 (1H, s), 7.2-7.55 (11H, m), 7.60 (1H, s), 8.43 (1H, d, J = 4.0 Hz). |
| 13 | 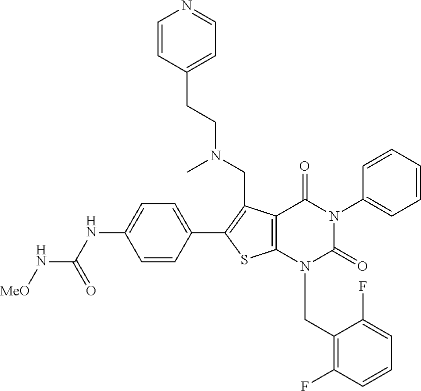 [SMILES: CONC(=O)Nc1ccc(-c2sc3c(c2CN(C)CCc2ccncc2)c(=O)n(-c2ccccc2)c(=O)n3Cc2c(F)cccc2F)cc1] | ¹H-NMR (CDCl₃) δ: 2.18 (3H, s), 2.68 (4H, s), 3.83 (5H, s), 5.37 (2H, s), 6.85-7.0 (5H, m), 7.16 (1H, s), 7.2-7.65 (10H, m), 8.35-8.4 (2H, m). |
| 14 | 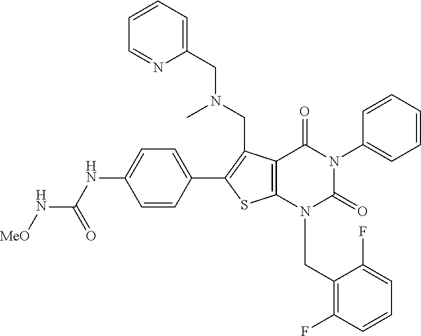 [SMILES: CONC(=O)Nc1ccc(-c2sc3c(c2CN(C)Cc2ccccn2)c(=O)n(-c2ccccc2)c(=O)n3Cc2c(F)cccc2F)cc1] | ¹H-NMR (CDCl₃) δ: 2.10 (3H, s), 3.71 (2H, s), 3.83 (3H, s), 3.99 (2H, s), 5.36 (2H, s), 6.92 (2H, t, J = 8.2 Hz), 7.0-7.1 (1H, m), 7.15 (1H, s), 7.2-7.35 (4H, m), 7.4-7.65 (9H, m), 8.4-8.5 (1H, m). |
| 15 | 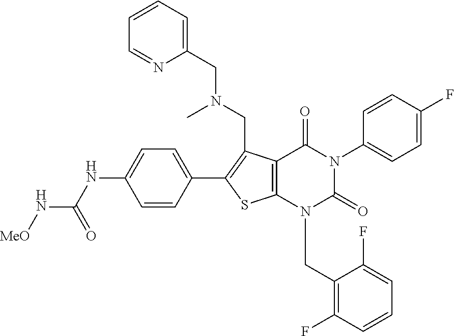 [SMILES: CONC(=O)Nc1ccc(-c2sc3c(c2CN(C)Cc2ccccn2)c(=O)n(-c2ccc(F)cc2)c(=O)n3Cc2c(F)cccc2F)cc1] | ¹H-NMR (CDCl₃) δ: 2.10 (3H, s), 3.71 (2H, s), 3.83 (3H, s), 3.97 (2H, s), 5.35 (2H, s), 6.93 (2H, t, J = 8.2 Hz), 7.0-7.7 (14H, m), 8.4-8.5 (1H, m). |
| 16 | 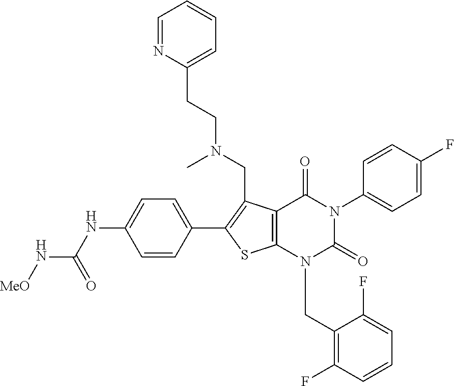 [SMILES: CONC(=O)Nc1ccc(-c2sc3c(c2CN(C)CCc2ccccn2)c(=O)n(-c2ccc(F)cc2)c(=O)n3Cc2c(F)cccc2F)cc1] | ¹H-NMR (CDCl₃) δ: 2.19 (3H, s), 2.85 (4H, s), 3.82 (5H, s), 5.36 (2H, s), 6.9-7.55 (13H, m), 7.60 (1H, s), 8.4-8.45 (1H, m). |
| 17 | 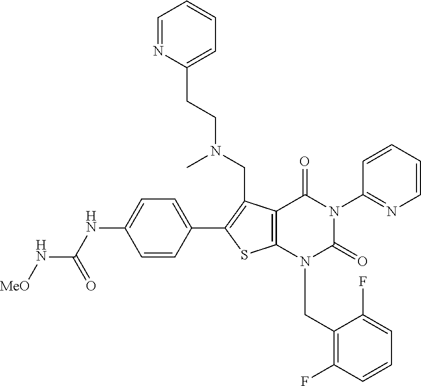 [SMILES: CONC(=O)Nc1ccc(-c2sc3c(c2CN(C)CCc2ccccn2)c(=O)n(-c2ccccn2)c(=O)n3Cc2c(F)cccc2F)cc1] | ¹H-NMR (CDCl₃) δ: 2.21 (3H, s), 2.8-2.9 (4H, m), 3.82 (5H, s), 5.34 (2H, brs), 6.85-7.75 (13H, m), 7.60 (1H, s),7.85- 7.95 (1H, m), 8.4-8.5 (1H, m), 8.65-8.75 (1H, m). |
| 18 | 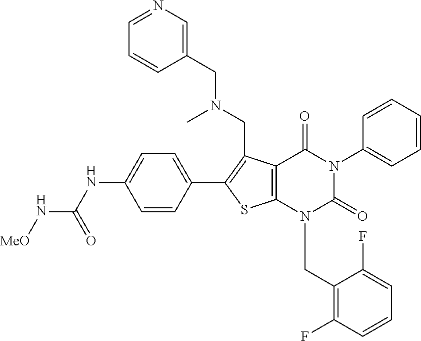 [SMILES: CONC(=O)Nc1ccc(-c2sc3c(c2CN(C)Cc2cccnc2)c(=O)n(-c2ccccc2)c(=O)n3Cc2c(F)cccc2F)cc1] | ¹H-NMR (CDCl₃) δ: 2.04 (3H, s), 3.58 (2H, s), 3.83 (3H, s), 3.92 (2H, s), 5.37 (2H, s), 6.92 (2H, t, J = 8.2 Hz), 7.05-7.7 (14H, m), 8.35-8.45 (2H, m). |
| 19 | 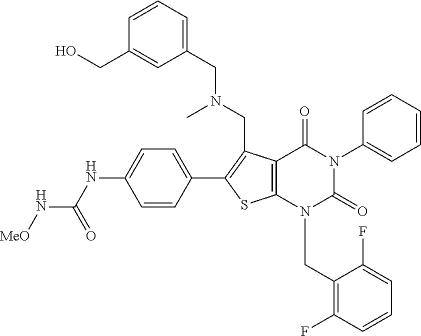 [SMILES: CONC(=O)Nc1ccc(-c2sc3c(c2CN(C)Cc2cccc(CO)c2)c(=O)n(-c2ccccc2)c(=O)n3Cc2c(F)cccc2F)cc1] | ¹H-NMR (CDCl₃) δ: 2.13 (3H, s), 3.72 (2H, s), 3.83 (3H, s), 3.97 (2H, s), 4.05-4.1 (1H, m), 4.65-4.75 (2H, m), 5.37 (2H, s), 6.93 (2H, t, J = 8.2 Hz), 6.9- 7.05 (1H, m), 7.1-7.2 (2H, m), 7.2-7.7 (12H, m). |
| 20 | 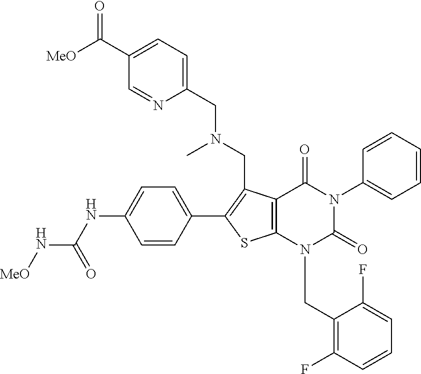 [SMILES: CONC(=O)Nc1ccc(-c2sc3c(c2CN(C)Cc2ccc(C(=O)OC)cn2)c(=O)n(-c2ccccc2)c(=O)n3Cc2c(F)cccc2F)cc1] | ¹H-NMR (CDCl₃) δ: 2.09 (3H, s), 3.76 (2H, s), 3.83 (3H, s), 3.93 (3H, s), 3.99 (2H, s), 5.35 (2H, s), 6.92 (2H, t, J = 8.2 Hz), 7.26 (1H, s), 7.2-7.6 (11H, m), 7.64 (1H, s), 8.05-8.15 (1H, m), 9.0-9.05 (1H, s). |
| 21 | 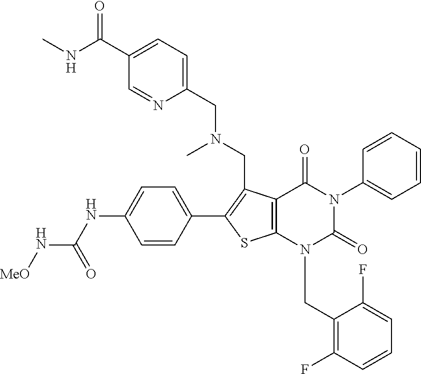 [SMILES: CNC(=O)c1ccc(CN(C)Cc2c(-c3ccc(NC(=O)NOC)cc3)sc3c2c(=O)n(-c2ccccc2)c(=O)n3Cc2c(F)cccc2F)nc1] | ¹H-NMR (CDCl₃) δ: 2.21 (3H, s), 3.00 (3H, d, J = 4.6 Hz), 3.74 (2H, s), 3.84 (3H, s), 3.92 (2H, s), 5.36 (2H, s), 6.5-6.65 (1H, m), 6.92 (2H, t, J = 8.0 Hz), 7.15-7.6 (12H, m), 7.71 (1H, s), 7.9-8.0 (1H, m), 8.80-8.85 (1H, m). |
| 22 | 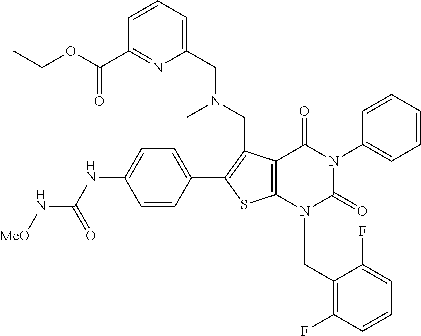 [SMILES: CCOC(=O)c1cccc(CN(C)Cc2c(-c3ccc(NC(=O)NOC)cc3)sc3c2c(=O)n(-c2ccccc2)c(=O)n3Cc2c(F)cccc2F)n1] | ¹H-NMR (CDCl₃) δ: 1.40 (3H, t, J = 7.2 Hz), 2.10 (3H, s), 3.79 (2H, s), 3.83 (3H, s), 4.01 (2H, s), 4.43 (2H, q, J = 7.2 Hz), 5.35 (2H, s), 6.92 (2H, t, J = 8.2 Hz), 7.15 (1H, s), 7.2-7.7 (13H, m), 7.88 (1H, d, J = 7.4 Hz). |
| 23 | 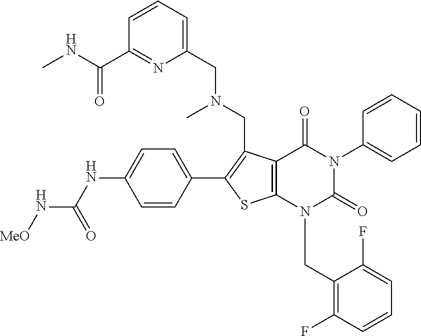 [SMILES: CNC(=O)c1cccc(CN(C)Cc2c(-c3ccc(NC(=O)NOC)cc3)sc3c2c(=O)n(-c2ccccc2)c(=O)n3Cc2c(F)cccc2F)n1] | ¹H-NMR (CDCl₃) δ: 2.13 (3H, s), 2.95 (3H, d, J = 5.2 Hz), 3.74 (2H, s), 3.84 (3H, s), 3.99 (2H, s), 5.36 (2H, s), 6.93 (2H, t, J = 8.2 Hz), 7.15-7.7 (13H, m), 7.9-8.1 (3H, m). |
| 24 | 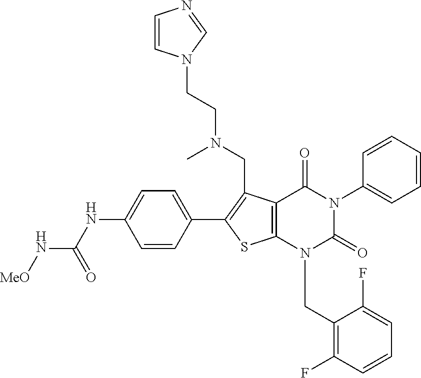 [SMILES: CONC(=O)Nc1ccc(-c2sc3c(c2CN(C)CCn2ccnc2)c(=O)n(-c2ccccc2)c(=O)n3Cc2c(F)cccc2F)cc1] | ¹H-NMR (CDCl₃) δ: 2.17 (3H, s), 2.6-2.8 (2H, m), 3.80 (2H, s), 3.83 (3H, s), 3.9-4.0 (2H, m), 5.38 (2H, s), 6.78 (1H, s), 6.85-7.0 (3H, m), 7.2-7.6 (12H m), 7.71 (1H, s). |
| 25 | 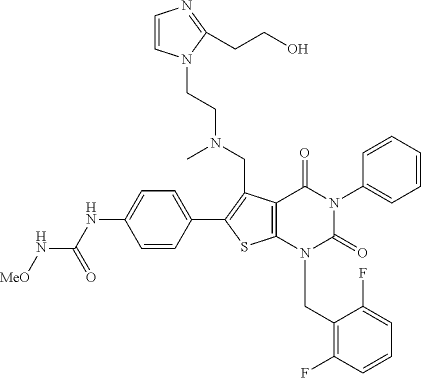 [SMILES: CONC(=O)Nc1ccc(-c2sc3c(c2CN(C)CCn2ccnc2CCO)c(=O)n(-c2ccccc2)c(=O)n3Cc2c(F)cccc2F)cc1] | ¹H-NMR (CDCl₃) δ: 1.9-2.1 (2H, m), 2.16 (3H, s), 2.6-2.8 (4H, m), 3.78 (2H, s), 3.83 (2H, s), 3.9-4.0 (2H, m), 5.38 (2H, s), 6.70 (1H, s), 6.80 (1H, s), 6.94 (2H, t, J = 8.0 Hz), 7.2- 7.6 (11H, m), 7.73 (1H, s). |
| 26 | 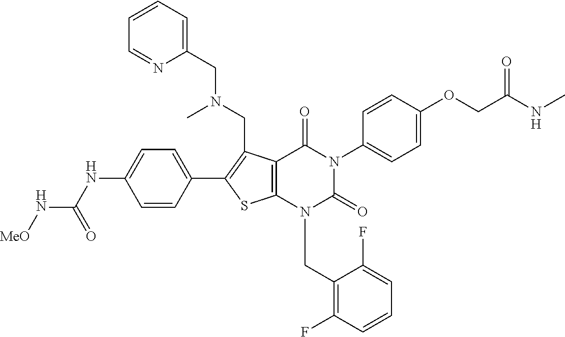 [SMILES: CNC(=O)COc1ccc(-n2c(=O)c3c(CN(C)Cc4ccccn4)c(-c4ccc(NC(=O)NOC)cc4)sc3n(Cc3c(F)cccc3F)c2=O)cc1] | ¹H-NMR (CDCl₃) δ: 2.09 (3H, s), 2.93 (3H, d, J = 4.8 Hz), 3.72 (2H, s), 3.83 (3H, s), 3.97 (2H, s), 4.55 (2H, s), 5.35 (2H, s), 6.55-6.65 (1H, m), 6.92 (2H, t, J = 8.0 Hz), 7.0-7.65 (14H, m), 8.44 (1H, d, J = 6.0 Hz). |
| 27 | 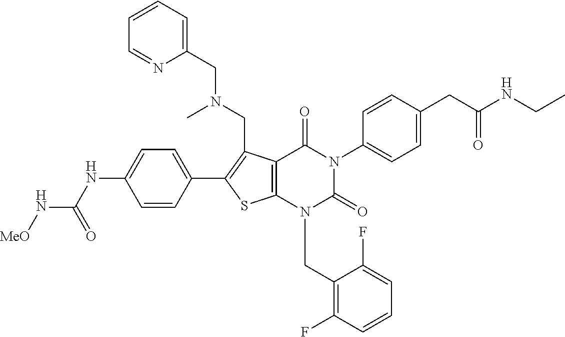 [SMILES: CCNC(=O)Cc1ccc(-n2c(=O)c3c(CN(C)Cc4ccccn4)c(-c4ccc(NC(=O)NOC)cc4)sc3n(Cc3c(F)cccc3F)c2=O)cc1] | ¹H-NMR (CDCl₃) δ: 1.10 (3H, t, J = 7.2 Hz), 2.10 (3H, s), 3.2- 3.4 (2H, m), 3.64 (2H, s), 3.72 (2H, s), 3.83 (3H, s), 3.98 (2H, s), 5.36 (2H, s), 5.45-5.55 (1H, m), 6.93 (2H, t, J = 8.0 Hz), 7.0- 7.1 (1H, m), 7.16 (1H, s), 7.25- 7.7 (12H, m), 8.44 (1H, d, J = 4.0 Hz). |
| 28 | 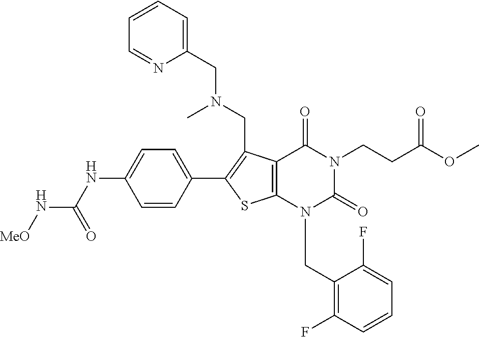 [SMILES: CONC(=O)Nc1ccc(-c2sc3c(c2CN(C)Cc2ccccn2)c(=O)n(CCC(=O)OC)c(=O)n3Cc2c(F)cccc2F)cc1] | ¹H-NMR (CDCl₃) δ: 2.10 (3H, s), 2.65-2.8 (2H, m), 3.68 (3H, s), 3.72 (2H, s), 3.83 (3H, s), 3.98 (2H, s), 4.3-4.5 (2H, m), 5.31 (2H, s), 6.91 (2H, t, J = 7.6 Hz), 7.0-7.4 (5H, m), 7.45-7.65 (5H, m), 8.4-8.5 (1H, m). |
| 29 | 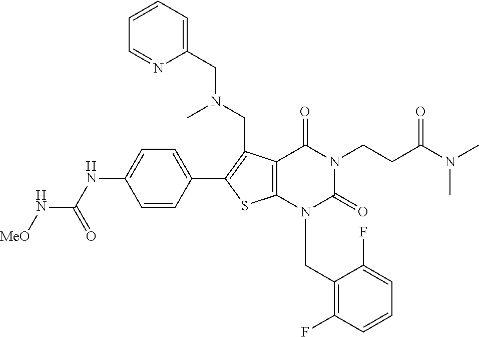 [SMILES: CONC(=O)Nc1ccc(-c2sc3c(c2CN(C)Cc2ccccn2)c(=O)n(CCC(=O)N(C)C)c(=O)n3Cc2c(F)cccc2F)cc1] | ¹H-NMR (CDCl₃) δ: 2.09 (3H, s), 2.75 (3H, d, J = 7.8 Hz), 2.94 (3H, s), 3.02 (3H, s), 3.73 (2H, s), 3.83 (3H, s), 4.07 (2H, s), 4.40 (2H, t, J = 7.8 Hz), 5.33 (2H, s), 6.91 (2H, t, J = 8.2 Hz), 7.0-7.35 (5H, m), 7.5-7.65 (5H, m), 8.45 (1H, d, J = 4.0 Hz). |
| 30 | 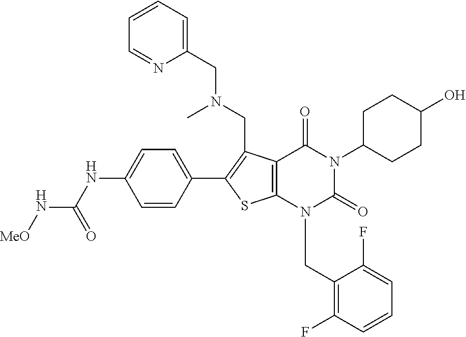 [SMILES: CONC(=O)Nc1ccc(-c2sc3c(c2CN(C)Cc2ccccn2)c(=O)n(C2CCC(O)CC2)c(=O)n3Cc2c(F)cccc2F)cc1] | ¹H-NMR (CDCl₃) δ: 1.4-1.8 (4H, m), 2.0-2.1 (2H, m), 2.12 (3H, s), 2.55-2,75 (2H, m), 3.73 (2H, s), 3.7-3.8 (1H, m), 3.82 (3H, s), 3.98 (2H, s), 4.9- 5.1 (1H, brm), 5.29 (2H, s), 6.91 (2H, t, J = 8.2 Hz), 7.0-7.4 (7H, m), 7.45-7.65 (5H, m), 8.45 (1H, d, J = 4.8 Hz). |
| 31 | 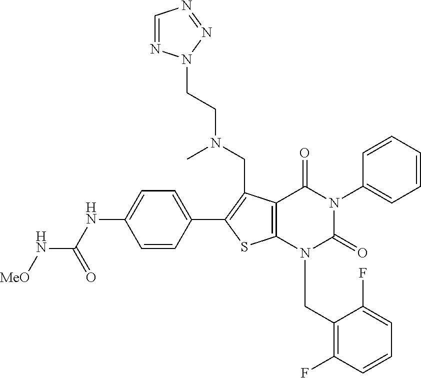 [SMILES: CONC(=O)Nc1ccc(-c2sc3c(c2CN(C)CCn2ncnn2)c(=O)n(-c2ccccc2)c(=O)n3Cc2c(F)cccc2F)cc1] | 2-yl Form (1) ¹H-NMR (CDCl₃) δ: 2.25 (3H, s), 3.04 (2H, t, J = 6.2 Hz), 3.78 (2H, s), 3.83 (3H, s), 4.66 (2H, t, J = 6.2 Hz), 5.38 (2H, s), 6.94 (2H, t, J = 8.0 Hz), 7.16 (1H, s), 7.2-7.6 (10H, m), 7.64 (1H, s), 8.30 (1H, s). |
| | 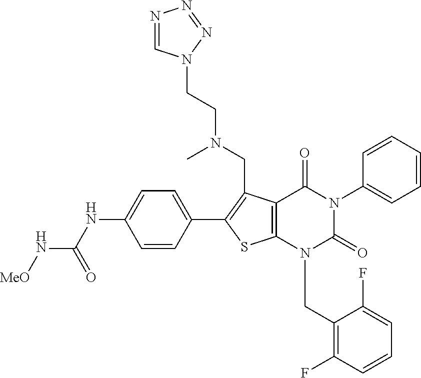 [SMILES: CONC(=O)Nc1ccc(-c2sc3c(c2CN(C)CCn2cnnn2)c(=O)n(-c2ccccc2)c(=O)n3Cc2c(F)cccc2F)cc1] | 1-yl Form (2) ¹H-NMR (CDCl₃) δ: 2.02 (3H, s), 2.7-2.8 (2H, m), 3.78 (2H, s), 3.83 (3H, s), 4.4-4.5 (2H, m), 5.38 (2H, s), 6.92 (2H, t, J = 8.0 Hz), 7.17 (1H, s), 7.25- 7.65 (10H, m), 7.66 (1H, s). |
| 32 | 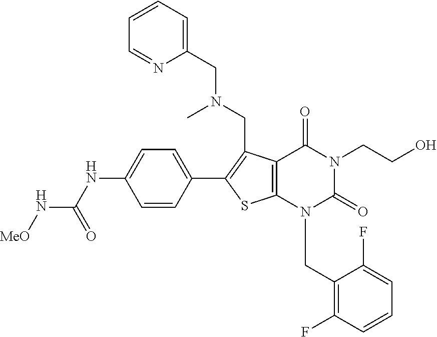 [SMILES: CONC(=O)Nc1ccc(-c2sc3c(c2CN(C)Cc2ccccn2)c(=O)n(CCO)c(=O)n3Cc2c(F)cccc2F)cc1] | ¹H-NMR (CDCl₃) δ: 2.22 (3H, s), 3.3-3.5 (1H, br), 3.78 (2H, s), 3.83 (3H, s), 3.9-4.05 (2H, m), 3.99 (2H, s), 4.35-4.4 (2H, m), 5.27 (2H, s), 6.91 (2H, t, J = 8.0 Hz), 6.9-7.1 (1H, m), 7.15 (1H, s), 7.2-7.65 (8H, m), 8.35-8.4 (1H, m). |
| 33 | 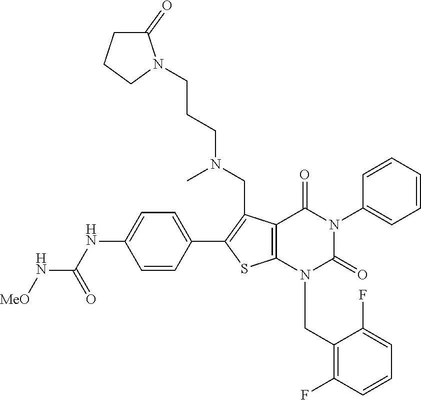 [SMILES: CONC(=O)Nc1ccc(-c2sc3c(c2CN(C)CCCN2CCCC2=O)c(=O)n(-c2ccccc2)c(=O)n3Cc2c(F)cccc2F)cc1] | ¹H-NMR (CDCl₃) δ: 1.5-1.7 (2H, m), 1.8-2.0 (2H, m),2.05 (3H, s), 2.25-2.45 (4H, m), 3.15 (2H, t, J = 7.8 Hz), 3.23 (2H, t, J = 7.2 Hz), 3.76 (2H, s), 3.83 (3H, s), 5.37 (2H, s), 6.93 (2H, t, J = 8.0 Hz), 7.15 (1H, s), 7.2-7.6 (10H, m), 7.67 (1H, s). |
| 34 | 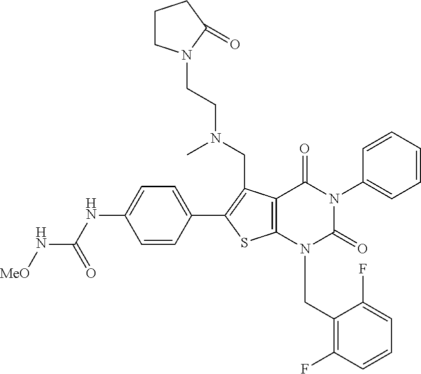 [SMILES: CONC(=O)Nc1ccc(-c2sc3c(c2CN(C)CCN2CCCC2=O)c(=O)n(-c2ccccc2)c(=O)n3Cc2c(F)cccc2F)cc1] | ¹H-NMR (CDCl₃) δ: 1.7-1.85 (2H, m), 2.14 (3H, s), 2.21 (2H, t, J = 8.1 Hz), 2.54 (2H, t, J = 6.2 Hz), 3.19 (2H, t, J = 7.0 Hz), 3.29 (2H, t, J = 6.2 Hz), 3.77 (2H, s), 3.83 (3H, s), 5.38 (2H, s), 6.93 (2H, t, J = 8.1 Hz), 7.18 (1H, s), 7.25-7.7 (11H, m). |
| 35 | 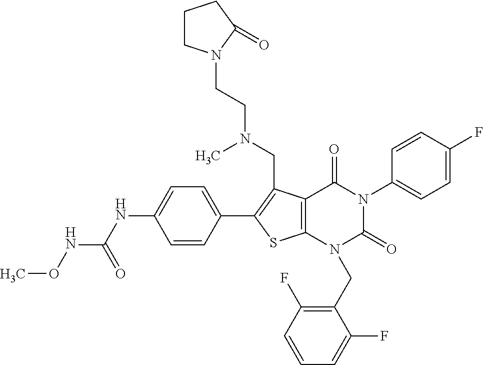 [SMILES: CONC(=O)Nc1ccc(-c2sc3c(c2CN(C)CCN2CCCC2=O)c(=O)n(-c2ccc(F)cc2)c(=O)n3Cc2c(F)cccc2F)cc1] | ¹H-NMR (CDCl₃) δ: 1.7-1.9 (2H, m), 2.15 (3H, s), 2.15-2.3 (2H, m), 2.52 (2H, t, J = 6.2 Hz), 3.20 (2H, t, J = 6.8 Hz), 3.29 (2H, t, J = 6.2 Hz), 3.77 (2H, s), 3.83 (3H, s), 5.37 (2H, s), 6.93 (2H, t, J = 8.2 Hz), 7.1-7.35 (6H, m), 7.5-7.65 (4H, m), 7.64 (1H, s). |
| 36 | 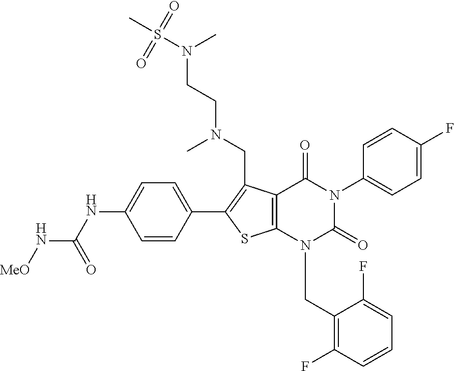 [SMILES: CONC(=O)Nc1ccc(-c2sc3c(c2CN(C)CCN(C)S(C)(=O)=O)c(=O)n(-c2ccc(F)cc2)c(=O)n3Cc2c(F)cccc2F)cc1] | ¹H-NMR (CDCl₃) δ: 2.13 (3H, s), 2.55 (2H, t, J = 6.4 Hz), 2.70 (3H, s), 2.71 (3H, s), 3.15 (2H, t, J = 6.4 Hz), 3.81 (2H, s), 3.83 (3H, s), 5.37 (2H, s), 6.93 (2H, t, J = 8.2 Hz), 7.1-7.7 (11H, m). |
| 37 | 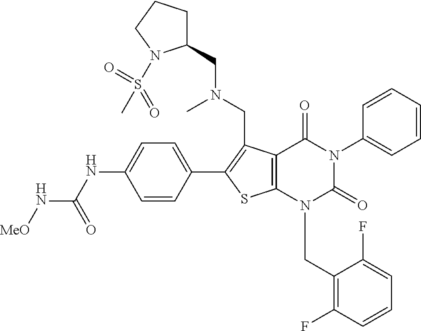 [SMILES: CONC(=O)Nc1ccc(-c2sc3c(c2CN(C)C[C@@H]2CCCN2S(C)(=O)=O)c(=O)n(-c2ccccc2)c(=O)n3Cc2c(F)cccc2F)cc1] | ¹H-NMR (CDCl₃) δ: 1.7-1.85 (4H, m), 2.08 (3H, s), 2.2-2.4 (1H, m), 2.5-2.65 (1H, m), 2.72 (3H, s), 3.15-3.3 (2H, m), 3.7- 3.9 (3H, m), 3.83 (3H, s), 5.37 (2H, s), 6.92 (2H, t, J = 8.2 Hz), 7.13 (1H, s), 7.2-7.7 (11H, m). |
| 38 | 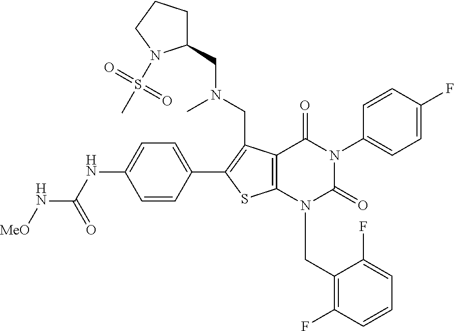 [SMILES: CONC(=O)Nc1ccc(-c2sc3c(c2CN(C)C[C@@H]2CCCN2S(C)(=O)=O)c(=O)n(-c2ccc(F)cc2)c(=O)n3Cc2c(F)cccc2F)cc1] | ¹H-NMR (CDCl₃) δ: 1.7-1.85 (4H, m), 2.09 (3H, s), 2.25- 2.35 (1H, m), 2.55-2.65 (1H, m), 2.71 (3H, s), 3.15-3.3 (2H, m), 3.65-3.7 (2H, m), 3.74 (1H, d, J = 12.0 Hz), 3.83 (3H, s), 3.87 (1H, d, J = 12.0 Hz), 5.36 (2H, s), 6.92 (2H, t, J = 8.2 Hz), 6.85-6.95 (1H, m), 7.1-7.35 (5H, m), 7.49 (2H, d, J = 8.4 Hz), 7.55 (2H, d, J = 8.4 Hz), 7.62 (1H, s). |
| 39 | 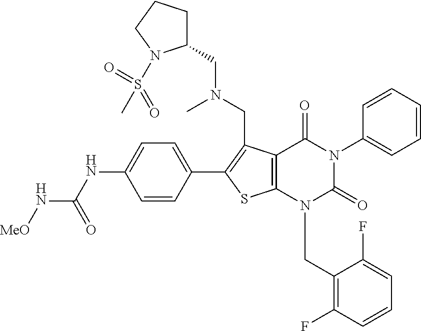 [SMILES: CONC(=O)Nc1ccc(-c2sc3c(c2CN(C)C[C@H]2CCCN2S(C)(=O)=O)c(=O)n(-c2ccccc2)c(=O)n3Cc2c(F)cccc2F)cc1] | ¹H-NMR (CDCl₃) δ: 1.7-1.85 (4H, m), 2.08 (3H, s), 2.2-2.4 (1H, m), 2.5-2.65 (1H, m), 2.72 (3H, s), 3.15-3.3 (2H, m), 3.7- 3.9 (3H, m), 3.83 (3H, s), 5.37 (2H, s), 6.92 (2H, t, J = 8.2 Hz), 7.13 (1H, s), 7.2-7.7 (11H, m). |
| 40 | 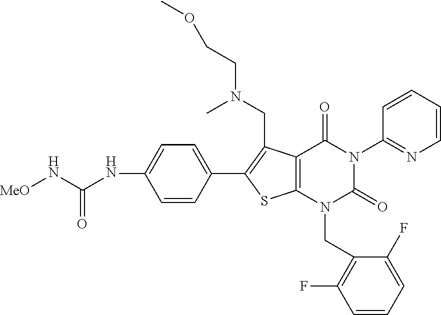 [SMILES: COCCN(C)Cc1c(-c2ccc(NC(=O)NOC)cc2)sc2c1c(=O)n(-c1ccccn1)c(=O)n2Cc1c(F)cccc1F] | ¹H-NMR (CDCl₃) δ: 2.15 (3H, s), 2.62 (2H, t, J = 5.9 Hz), 3.26 (3H, s), 3.41 (2H, t, J = 5.9 Hz), 3.80 (3H, s), 3.81 (2H, brs), 5.34 (2H, brs), 6.91 (2H, t, J = 8.1 Hz), 7.24-7.40 (4H, m), 7.53 (2H, d, J = 8.4 Hz), 7.62 (2H, d, J = 8.4 Hz), 7.65 (1H, s), 7.88 (1H, dt, J = 1.5 Hz, 7.8 Hz), 8.67-8.69 (1H, m). |
| 41 | 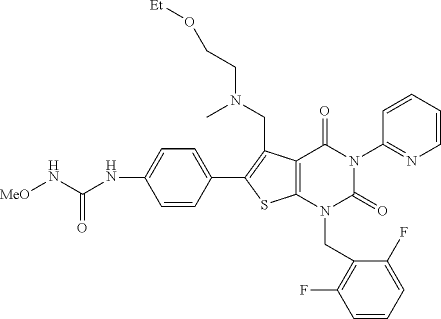 [SMILES: CCOCCN(C)Cc1c(-c2ccc(NC(=O)NOC)cc2)sc2c1c(=O)n(-c1ccccn1)c(=O)n2Cc1c(F)cccc1F] | ¹H-NMR (CDCl₃) δ: 1.13 (3H, t, J = 6.9 Hz), 2.15 (3H, s), 2.63 (2H, t, J = 6.2 Hz), 3.39 (2H, q, J = 6.9 Hz), 3.44 (2H, t, J = 6.2 Hz), 3.80 (2H, brs), 3.81 (3H, s), 5.34 (2H, brs), 6.91 (2H, t, J = 8.1 Hz), 7.19 (1 H, s), 7.27- 7.32 (1H, m), 7.35-7.41 (2H, m), 7.53 (2H, d, J = 8.4 Hz), 7.63 (1H, s), 7.64 (2H, d, J = 8.4 Hz), 7.88 (1H, dt, J = 1.2 Hz, 7.5 Hz), 8.68 (1H, dt, J = 0.9 Hz, 4.8 Hz). |
| 42 | 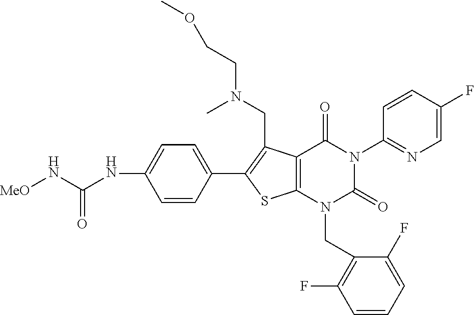 [SMILES: COCCN(C)Cc1c(-c2ccc(NC(=O)NOC)cc2)sc2c1c(=O)n(-c1ccc(F)cn1)c(=O)n2Cc1c(F)cccc1F] | ¹H-NMR (CDCl₃) δ: 2.13 (3H, s), 2.62 (2H, t, J = 5.9 Hz), 3.26 (3H, s), 3.41 (2H, t, J = 5.9 Hz), 3.80 (2H, brs), 3.82 (3H, s), 5.33 (2H, brs), 6.92 (2H, t, J = 8.3 Hz), 7.19 (1H, s), 7.28- 7.38 (2H, m), 7.52-7.63 (6H, m), 8.51 (1H, d, J = 3.0 Hz). |
| 43 | 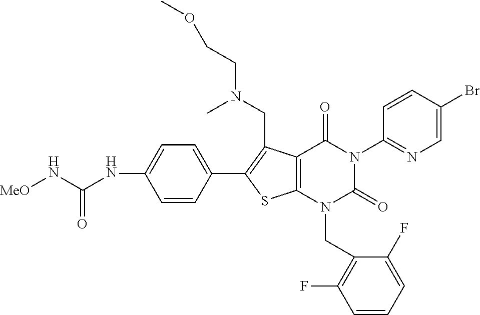 [SMILES: COCCN(C)Cc1c(-c2ccc(NC(=O)NOC)cc2)sc2c1c(=O)n(-c1ccc(Br)cn1)c(=O)n2Cc1c(F)cccc1F] | ¹H-NMR (CDCl₃) δ: 2.13 (3H, s), 2.62 (2H, t, J = 5.9 Hz), 3.26 (3H, s), 3.41 (2H, t, J = 5.9 Hz), 3.78 (2H, brs), 3.80 (3H, s), 5.32 (2H, brs), 6.92 (2H, t, J = 8.1 Hz), 7.27 (1H, d, J = 8.4 Hz), 7.27-7.33 (1H, m), 7.37 (1H, s), 7.54 (2H, d, J = 9.0 Hz), 7.60 (2H, d, J = 9.0 Hz), 7.64 (1H, s), 7.98 (1H, dd, J = 2.7 Hz, 8.4 Hz), 8.72 (1H, d, J = 2.7 Hz). |
| 44 | 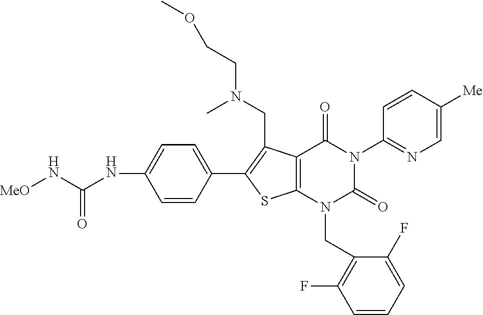 [SMILES: COCCN(C)Cc1c(-c2ccc(NC(=O)NOC)cc2)sc2c1c(=O)n(-c1ccc(C)cn1)c(=O)n2Cc1c(F)cccc1F] | ¹H-NMR (CDCl₃) δ: 2.14 (3H, s), 2.39 (3H, s), 2.62 (2H, t, J = 5.7 Hz), 3.26 (3H, s), 3.41 (2H, t, J = 5.7 Hz), 3.77 (2H, brs), 3.80 (3H, s), 5.26 (1H, brs), 5.38 (1H, brs), 6.91 (2H, t, J = 8.3 Hz), 7.23-7.34 (2H, m), 7.42 (1H, s), 7.53 (2H, d, J = 8.7 Hz), 7.62 (2H, d, J = 8.7 Hz), 7.66 (1H, s), 7.66-7.69 (1H, m), 8.48 (1H, d, J = 2.4 Hz). |
| 45 | 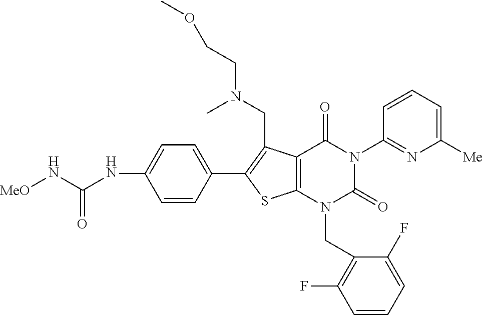 [SMILES: COCCN(C)Cc1c(-c2ccc(NC(=O)NOC)cc2)sc2c1c(=O)n(-c1cccc(C)n1)c(=O)n2Cc1c(F)cccc1F] | ¹H-NMR (CDCl₃) δ: 2.15 (3H, s), 2.60 (3H, s), 2.62 (2H, t, J = 5.8 Hz), 3.27 (3H, s), 3.41 (2H, t, J = 5.8 Hz), 3.66-3.94 (2H, m), 3.81 (3H, s), 5.15 (1H, d, J = 15.3 Hz), 5.48 (1H, d, J = 15.3 Hz), 6.91 (2H, t, J = 8.1 Hz), 7.16 (1H, d, J = 7.8 Hz), 7.21 (1H, d, J = 7.8 Hz), 7.25 (1H, s), 7.26-7.35 (1H, m), 7.53 (2H, d, J = 8.7 Hz), 7.63 (1H, s), 7.53 (2H, d, J = 8.7 Hz), 7.64 (2H, d, J = 8.7 Hz), 7.76 (1H, t, J = 7.8 Hz). |
| 46 | 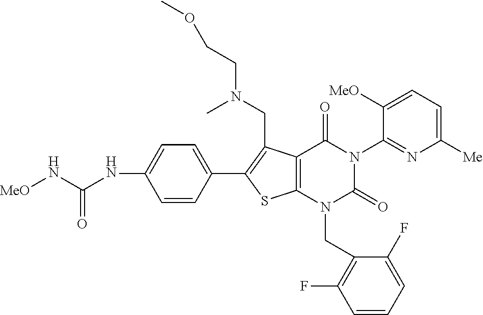 [SMILES: COCCN(C)Cc1c(-c2ccc(NC(=O)NOC)cc2)sc2c1c(=O)n(-c1nc(C)ccc1OC)c(=O)n2Cc1c(F)cccc1F] | ¹H-NMR (CDCl₃) δ: 2.13 (3H, s), 2.51 (3H, s), 2.62 (2H, t, J = 5.9 Hz), 3.26 (3H, s), 3.40 (2H, t, J = 5.9 Hz), 3.77 (1H, d, J = 12.3 Hz), 3.77 (3H, s), 3.79 (3H, s), 3.86 (1H, d, J = 12.3 Hz), 5.24 (1H, d, J = 15.6 Hz), 5.40 (1H, d, J = 15.6 Hz), 6.90 (2H, t, J = 8.1 Hz), 7.19 (1H, d, J = 8.4 Hz), 7.23-7.34 (1H, m), 7.27 (1H, d, J = 8.4 Hz), 7.51 (2H, d, J = 8.7 Hz), 7.58 (2H, d, J = 8.7 Hz), 7.65 (1H, s), 7.69 (1H, s). |
| 47 | 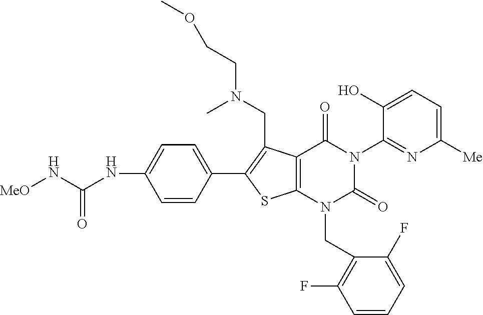 [SMILES: COCCN(C)Cc1c(-c2ccc(NC(=O)NOC)cc2)sc2c1c(=O)n(-c1nc(C)ccc1O)c(=O)n2Cc1c(F)cccc1F] | ¹H-NMR (CDCl₃) δ: 2.11 (3H, s), 2.48 (3H, s), 2.51-2.59 (2H, m), 3.20 (3H, s), 3.30-3.46 (4H, m), 3.60 (1H, d, J = 12.3 Hz), 3.79 (3H, s), 4.05 (1H, d, J = 12.3 Hz), 5.21 (1H, d, J = 15.6 Hz), 5.31 (1H, d, J = 15.6 Hz), 6.88 (2H, t, J = 8.1 Hz), 7.07 (1H, d, J = 8.1 Hz), 7.21-7.31 (2H, m), 7.43-7.51 (4H, m), 7.69 (1H, s). |
| 48 | 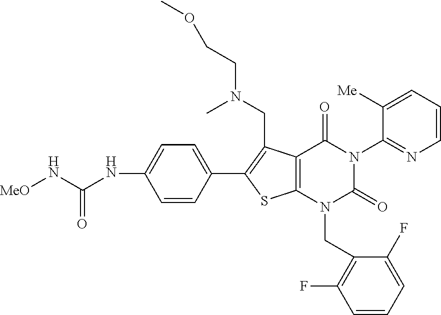 [SMILES: COCCN(C)Cc1c(-c2ccc(NC(=O)NOC)cc2)sc2c1c(=O)n(-c1ncccc1C)c(=O)n2Cc1c(F)cccc1F] | ¹H-NMR (CDCl₃) δ: 2.20 (3H, s), 2.63 (2H, t, J = 6.2 Hz), 3.28 (3H, s), 3.41 (2H, t, J = 6.2 Hz), 3.78 (1H, d, J = 12.3 Hz), 3.82 (3H, s), 3.84 (3H, s), 3.88 (1H, d, J = 12.3 Hz), 5.35 (2H, s), 6.92 (2H, t, J = 8.1 Hz), 7.17 (1H, s), 7.23-7.39 (3H, m), 7.54 (2H, d, J = 8.8 Hz), 7.57 (1H, s), 7.69 (2H, d, J = 8.8 Hz), 8.23-8.27 (1H, m). |
| 49 | 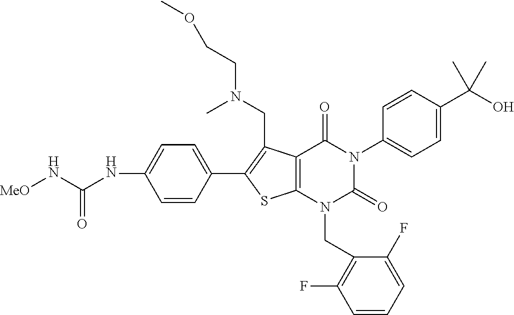 [SMILES: COCCN(C)Cc1c(-c2ccc(NC(=O)NOC)cc2)sc2c1c(=O)n(-c1ccc(C(C)(C)O)cc1)c(=O)n2Cc1c(F)cccc1F] | ¹H-NMR (CDCl₃) δ: 1.60 (6H, s), 1.79 (1H, s), 2.14 (3H, s), 2.63 (2H, t, J = 5.9 Hz), 3.27 (3H, s), 3.41 (2H, t, J = 5.9 Hz), 3.81 (3H, s), 3.82 (2H, s), 5.36 (2H, s), 6.92 (2H, t, J = 8.3 Hz), 7.20-7.34 (4H, m), 7.53 (2H, d, J = 8.7 Hz), 7.60-7.63 (5H, m). |
| 50 | 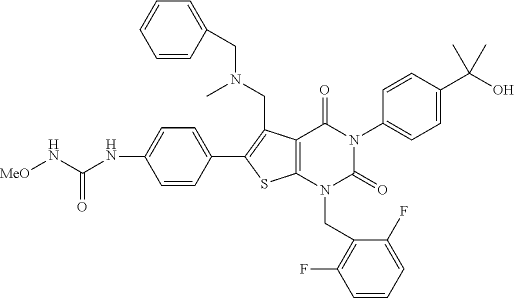 [SMILES: CONC(=O)Nc1ccc(-c2sc3c(c2CN(C)Cc2ccccc2)c(=O)n(-c2ccc(C(C)(C)O)cc2)c(=O)n3Cc2c(F)cccc2F)cc1] | ¹H-NMR (CDCl₃) δ: 1.62 (6H, s), 1.77 (1H, s), 2.05 (3H, s), 3.56 (2H, s), 3.82 (3H, s), 3.90 (2H, s), 5.36 (2H, s), 6.91 (2H, t, J = 8.1 Hz), 7.14-7.38 (9H, m), 7.55 (2H, d, J = 9.0 Hz), 7.62 (1H, s), 7.64 (2H, d, J = 8.7 Hz), 7.72 (2H, d, J = 8.4 Hz). |
| 51 | 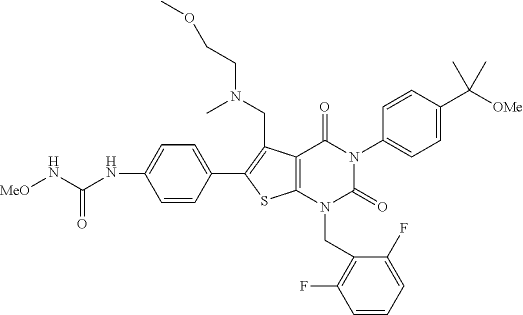 [SMILES: COCCN(C)Cc1c(-c2ccc(NC(=O)NOC)cc2)sc2c1c(=O)n(-c1ccc(C(C)(C)OC)cc1)c(=O)n2Cc1c(F)cccc1F] | ¹H-NMR (CDCl₃) δ: 1.55 (6H, s), 2.15 (3H, s), 2.64 (2H, t, J = 5.9 Hz), 3.11 (3H, s), 3.27 (3H, s), 3.41 (2H, t, J = 5.9 Hz), 3.82 (3H, s), 3.83 (2H, s), 5.36 (2H, s), 6.92 (2H, t, J = 8.3 Hz), 7.16 (1H, s), 7.24-7.36 (4H, m), 7.51-7.63 (6H, m). |
| 52 | 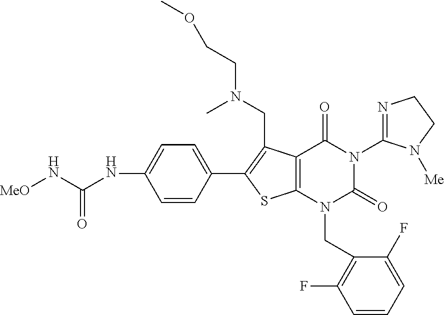 [SMILES: COCCN(C)Cc1c(-c2ccc(NC(=O)NOC)cc2)sc2c1c(=O)n(C1=NCCN1C)c(=O)n2Cc1c(F)cccc1F] | ¹H-NMR (CDCl₃) δ: 2.15 (3H, s), 2.61 (2H, dt, J = 1.8 Hz, 6.90 Hz), 3.27 (3H, s), 3.40 (2H, dt, J = 1.8 Hz, 6.0 Hz), 3.53 (3H, s), 3.75 (1H, d, J = 12.3 Hz), 3.80 (3H, s), 3.81 (1H, d, J = 12.3 Hz), 5.12 (1H, d, J = 15.9 Hz), 5.57 (1H, d, J = 15.9 Hz), 6.91 (2H, t, J = 8.1 Hz), 6.99 (1H, d, J = 1.5 Hz), 7.14 (1H, d, J = 1.5 Hz), 7.28 (1H, s), 7.25-7.34 (1H, m), 7.53 (2H, d, J = 9.0 Hz), 7.60 (2H, d, J = 9.0 Hz), 7.70 (1H, s). |
| 53 | 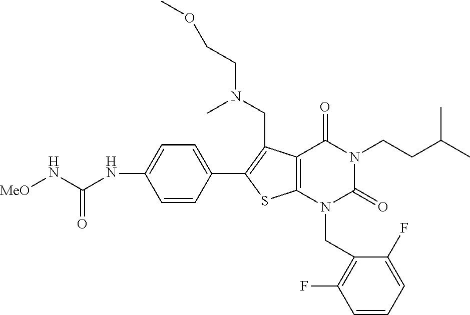 [SMILES: COCCN(C)Cc1c(-c2ccc(NC(=O)NOC)cc2)sc2c1c(=O)n(CCC(C)C)c(=O)n2Cc1c(F)cccc1F] | ¹H-NMR (CDCl₃) δ: 0.98 (6H, d, J = 6.3 Hz), 1.52-1.58 (2H, m), 1.64-1.71 (1H, m), 2.14 (3H, s), 2.66 (2H, t, J = 5.9 Hz), 3.30 (3H, s), 3.45 (2H, t, J = 5.9 Hz), 3.81 (3H, s), 3.85 (2H, s), 4.04-4.09 (2H, m), 5.33 (2H, s), 6.90 (2H, t, J = 8.3 Hz), 7.17 (1H, s), 7.24-7.35 (1H, m), 7.51 (2H, d, J = 8.7 Hz), 7.57 (2H, d, J = 8.7 Hz), 7.60 (1H, s). |
| 54 | 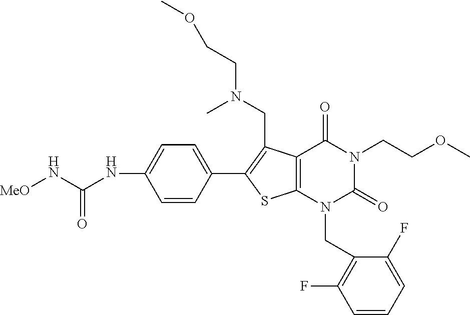 [SMILES: COCCN(C)Cc1c(-c2ccc(NC(=O)NOC)cc2)sc2c1c(=O)n(CCOC)c(=O)n2Cc1c(F)cccc1F] | ¹H-NMR (CDCl₃) δ: 2.14 (3H, s), 2.65 (2H, t, J = 5.9 Hz), 3.30 (3H, s), 3.36 (3H, s), 3.45 (2H, t, J = 5.9 Hz), 3.66 (2H, t, J = 5.9 Hz), 3.81 (3H, s), 3.84 (2H, s), 4.30 (2H, t, J = 5.9 Hz), 5.33 (2H, s), 6.90 (2H, t, J = 8.3 Hz), 7.15 (1H, s), 7.24-7.34 (1H, m), 7.51 (2H, d, J = 9.0 Hz), 7.56 (2H, d, J = 9.0 Hz), 7.60 (1H, m). |
| 55 | 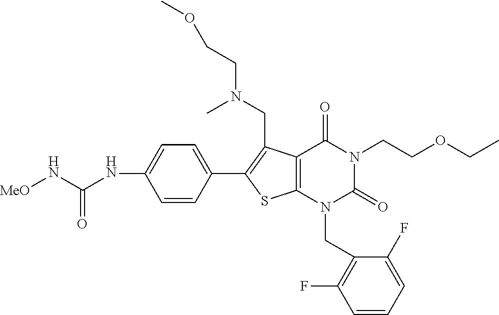 [SMILES: CCOCCn1c(=O)c2c(CN(C)CCOC)c(-c3ccc(NC(=O)NOC)cc3)sc2n(Cc2c(F)cccc2F)c1=O] | ¹H-NMR (CDCl₃) δ: 1.15 (3H, t, J = 6.9 Hz), 2.14 (3H, s), 2.66 (2H, t, J = 6.0 Hz), 3.30 (3H, s), 3.45 (2H, t, J = 6.0 Hz), 3.54 (2H, q, J = 6.9 Hz), 3.69 (2H, t, J = 6.0 Hz), 3.81 (3H, s), 3.84 (2H, s), 4.29 (2H, t, J = 6.0 Hz), 5.32 (2H, s), 6.89 (2H, t, J = 8.1 Hz), 7.17 (1H, s), 7.23-7.34 (1H, m), 7.52 (2H, d, J = 8.7 Hz), 7.57 (2H, d, J = 8.7 Hz), 7.60 (1H, m). |
| 56 | 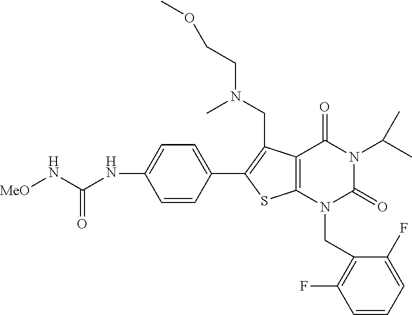 [SMILES: COCCN(C)Cc1c(-c2ccc(NC(=O)NOC)cc2)sc2c1c(=O)n(C(C)C)c(=O)n2Cc1c(F)cccc1F] | ¹H-NMR (CDCl₃) δ: 1.52 (6H, d, J = 6.9 Hz), 2.13 (3H, s), 2.66 (2H, t, J = 5.9 Hz), 3.31 (3H, s), 3.46 (2H, t, J = 5.9 Hz), 3.82 (3H, s), 3.84 (2H, s), 5.31 (2H, s), 5.34 (1H, m), 6.90 (2H, t, J = 8.1 Hz), 7.16 (1H, s), 7.24- 7.35 (1H, m), 7.52 (2H, d, J = 8.4 Hz), 7.55 (2H, d, J = 8.4 Hz), 7.60 (1H, m). |
| 57 | 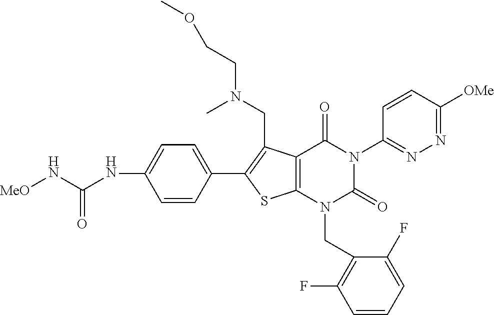 [SMILES: COCCN(C)Cc1c(-c2ccc(NC(=O)NOC)cc2)sc2c1c(=O)n(-c1ccc(OC)nn1)c(=O)n2Cc1c(F)cccc1F] | ¹H-NMR (CDCl₃) δ: 2.13 (3H, s), 2.62 (2H, t, J = 5.7 Hz), 3.26 (3H, s), 3.41 (2H, t, J = 5.7 Hz), 3.74 (2H, brs), 3.82 (3H, s), 4.18 (3H, s), 5.32 (2H, brs), 6.92 (2H, t, J = 8.3 Hz), 7.12 (1H, d, J = 9.3 Hz), 7.24 (1H, s) 7.29-7.35 (1H, m), 7.41 (2H, d, J = 9.3 Hz), 7.54 (2H, d, J = 9.0 Hz), 7.59 (2H, d, J = 8.7 Hz), 7.66 (1H, s). |
| 58 | 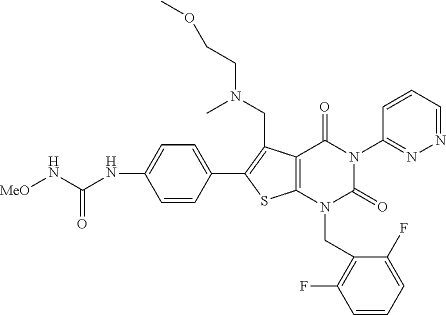 [SMILES: COCCN(C)Cc1c(-c2ccc(NC(=O)NOC)cc2)sc2c1c(=O)n(-c1cccnn1)c(=O)n2Cc1c(F)cccc1F] | ¹H-NMR (CDCl₃) δ: 2.12 (3H, s), 2.61 (2H, t, J = 5.7 Hz), 3.26 (3H, s), 3.39 (2H, t, J = 5.7 Hz), 3.78 (2H, brs), 3.82 (3H, s), 5.34 (2H, brs), 6.93 (2H, t, J = 8.1 Hz), 7.26 (1H, s), 7.29- 7.37 (1H, m), 7.53-7.61 (5H, m), 7.67 (1H, s), 7.69 (1H, dd, J = 4.8 Hz, 8.4 Hz), 9.28 (1H, dd, J = 1.8 Hz, 4.8 Hz). |
| 59 | 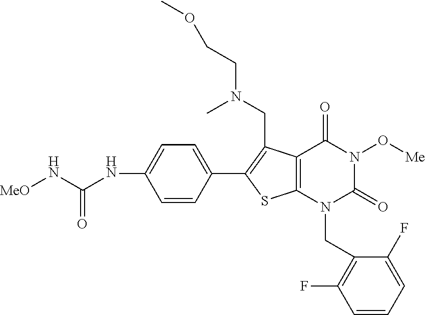 [SMILES: COCCN(C)Cc1c(-c2ccc(NC(=O)NOC)cc2)sc2c1c(=O)n(OC)c(=O)n2Cc1c(F)cccc1F] | ¹H-NMR (CDCl₃) δ: 2.14 (3H, s), 2.68 (2H, t, J = 6.0 Hz), 3.31 (3H, s), 3.47 (2H, t, J = 6.0 Hz), 3.82 (3H, s), 3.83 (2H, s), 4.06 (3H, s), 5.35 (2H, s), 6.92 (2H, t, J = 8.3 Hz), 7.20 (1H, s), 7.29-7.35 (1H, m), 7.55 (4H, s), 7.63 (1H, s). |
| 60 | 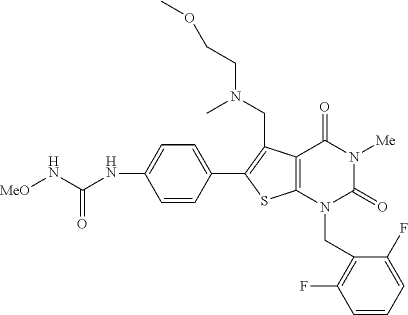 [SMILES: COCCN(C)Cc1c(-c2ccc(NC(=O)NOC)cc2)sc2c1c(=O)n(C)c(=O)n2Cc1c(F)cccc1F] | ¹H-NMR (CDCl₃) δ: 2.13 (3H, s), 2.66 (2H, t, J = 5.9 Hz), 3.31 (3H, s), 3.45 (3H, s), 3.48 (2H, t, J = 5.9 Hz), 3.82 (3H, s), 3.84 (2H, s), 5.33 (2H, s), 6.91 (2H, t, J = 8.3 Hz), 7.17 (1H, s), 7.25-7.35 (1H, m), 7.55 (4H, s), 7.62 (1H, s). |
| 61 | 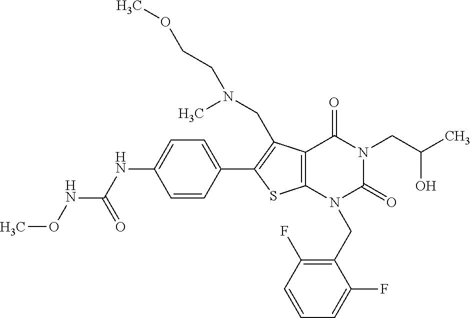 [SMILES: COCCN(C)Cc1c(-c2ccc(NC(=O)NOC)cc2)sc2c1c(=O)n(CC(C)O)c(=O)n2Cc1c(F)cccc1F] | ¹H-NMR (CDCl₃) δ: 1.27 (3H, d, J = 5.6 Hz), 2.12 (3H, s), 2.64 (2H, t, J = 5.8 Hz), 2.9-3.05 (1H, m), 3.30 (3H, s), 3.45 (2H, d, J = 5.8 Hz), 3.82 (5H, s), 4.05- 4.25 (1H, m), 4.18 (2H, s), 5.34 (2H, s), 6.91 (2H, t, J = 8.2 Hz), 7.2-7.4 (1H, m), 7.5-7.6 (3H, m), 7.63 (1H, s). |
| 62 | 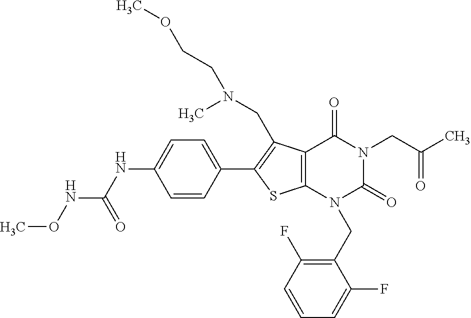 [SMILES: COCCN(C)Cc1c(-c2ccc(NC(=O)NOC)cc2)sc2c1c(=O)n(CC(C)=O)c(=O)n2Cc1c(F)cccc1F] | ¹H-NMR (CDCl₃) δ: 2.13 (3H, s), 2.27 (3H, s), 2.55-2.65 (2H, m), 3.29 (3H, s), 3.4-3.5 (2H, m), 3.82 (5H, s), 4.88 (2H, s), 5.33 (2H, s), 6.91 (2H, t, J = 8.0 Hz), 7.2-7.35 (1H, m), 7.5-7.65 (4H, m). |
| 63 | 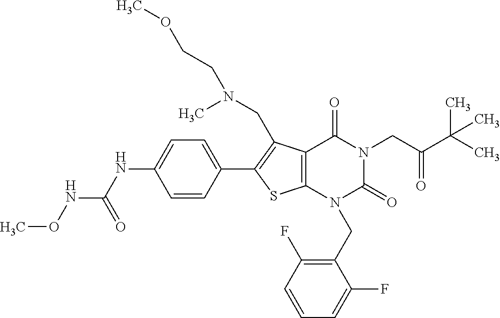 [SMILES: COCCN(C)Cc1c(-c2ccc(NC(=O)NOC)cc2)sc2c1c(=O)n(CC(=O)C(C)(C)C)c(=O)n2Cc1c(F)cccc1F] | ¹H-NMR (CDCl₃) δ: 1.30 (9H, s), 2.12 (3H, s), 2.62 (2H, t, J = 5.8 Hz), 3.29 (3H, s), 3.44 (2H, d, J = 5.8 Hz), 3.80 (2H, s), 3.82 (3H, s), 5.04 (2H, s), 5.33 (2H, s), 6.91 (2H, t, J = 8.2 Hz), 7.14 (1H, s), 7.2-7.3 (1H, m), 7.5-7.6 (4H, m), 7.61 (1H, s). |
| 64 | 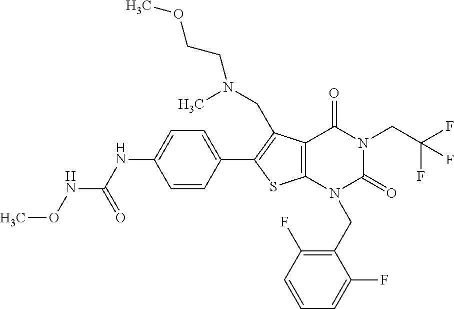 [SMILES: COCCN(C)Cc1c(-c2ccc(NC(=O)NOC)cc2)sc2c1c(=O)n(CC(F)(F)F)c(=O)n2Cc1c(F)cccc1F] | ¹H-NMR (CDCl₃) δ: 2.12 (3H, s), 2.65 (2H, t, J = 6.0 Hz), 3.31 (3H, s), 3.45 (2H, d, J = 6.0 Hz) 3.82 (5H, s), 4.75-4.85 (2H, m), 5.36 (2H, s), 6.92 (2H, t, J = 8.2 Hz), 7.13 (1H, s), 7.2- 7.35 (1H, m), 7.55-7.6 (4H, m) 7.62 (1H, s). |
| 65 | 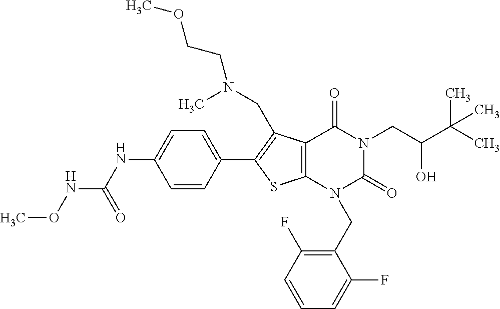 [SMILES: COCCN(C)Cc1c(-c2ccc(NC(=O)NOC)cc2)sc2c1c(=O)n(CC(O)C(C)(C)C)c(=O)n2Cc1c(F)cccc1F] | ¹H-NMR (CDCl₃) δ: 1.04 (9H, s), 2.12 (3H, s), 2.64 (2H, t, J = 6.0 Hz), 2.96 (1H, d, J = 6.0 Hz), 3.31 (3H, s), 3.46 (2H, d, J = 6.0 Hz), 3.5-3.6 (1H, m), 3.82 (3H, s), 3.75-3.9 (2H, m), 4.05-4.2 (1H, m), 4.3-4.45 (1H, m), 5.25-5.45 (2H, m), 6.91 (2H, t, J = 8.2 Hz), 7.14 (1H, s), 7.2-7.35 (1H, m), 7.5-7.6 (4H, m), 7.61 (1H, s). |
| 66 | 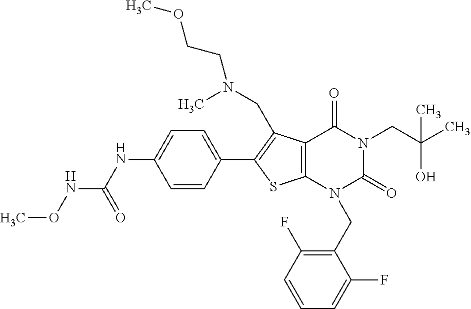 [SMILES: COCCN(C)Cc1c(-c2ccc(NC(=O)NOC)cc2)sc2c1c(=O)n(CC(C)(C)O)c(=O)n2Cc1c(F)cccc1F] | ¹H-NMR (CDCl₃) δ: 1.28 (6H, s), 2.3 (3H, s), 2.64 (2H, t, J = 5.8 Hz), 3.30 (3H, s), 3.45 (2H, t, J = 5.8 Hz), 3.82 (5H, s), 3.99 (1H, s), 4.25 (2H, s), 5.36 (2H, s), 6.91 (2H, t, J = 8.2 Hz), 7.12 (1H, s), 7.2-7.4 (1H, m), 7.5-7.6 (4H, m), 7.61 (1H, s). |
| 67 | 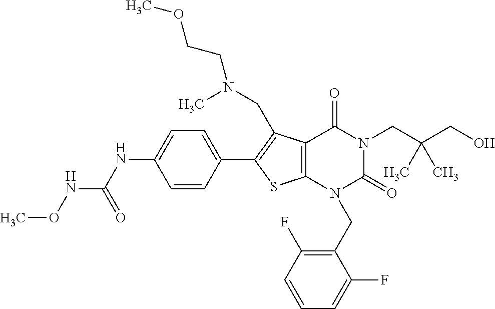 [SMILES: COCCN(C)Cc1c(-c2ccc(NC(=O)NOC)cc2)sc2c1c(=O)n(CC(C)(C)CO)c(=O)n2Cc1c(F)cccc1F] | ¹H-NMR (CDCl₃) δ: 0.96 (6H, s), 2.13 (3H, s), 2.64 (2H, t, J = 6.2 Hz), 3.13 (2H, s), 3.30 (3H, s), 3.45 (2H, t, J = 6.2 Hz) 3.82 (5H, s), 3.95-4.15 (2H, brm), 5.1-5.5 (2H, br), 6.91 (2H, t, J = 8.2 Hz), 7.14 (1H, s) 7.2-7.4 (1H, m), 7.5-7.6 (4H, m), 7.61 (1H, s). |
| 68 | 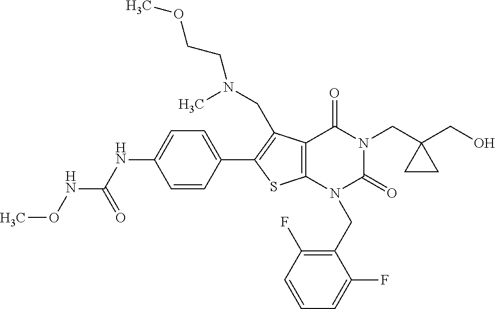 [SMILES: COCCN(C)Cc1c(-c2ccc(NC(=O)NOC)cc2)sc2c1c(=O)n(CC1(CO)CC1)c(=O)n2Cc1c(F)cccc1F] | ¹H-NMR (CDCl₃) δ: 0.46 (2H, t, J = 5.4 Hz), 0.85 (2H, t, J = 5.4 Hz), 2.11 (3H, s), 2.64 (2H, t, J = 6.0 Hz), 3.25 (2H, s), 3.31 (3H, s), 3.46 (2H, t, J = 6.0 Hz), 3.82 (5H, s), 3.95-4.15 (1H, br), 4.14 (2H, s), 5.37 (2H, s), 6.91 (2H, t, J = 8.0 Hz), 7.12 (1H, s), 7.2-7.4 (1H, m), 7.54 (4H, s), 7.61 (1H, s). |
| 69 | 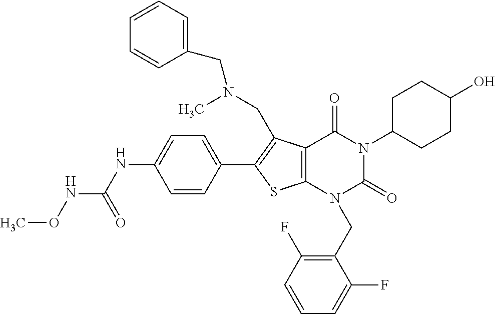 [SMILES: CONC(=O)Nc1ccc(-c2sc3c(c2CN(C)Cc2ccccc2)c(=O)n(C2CCC(O)CC2)c(=O)n3Cc2c(F)cccc2F)cc1] | ¹H-NMR (CDCl₃) δ: 1.35-1.65 (3H, m), 1.65-1.8 (2H, m), 2.07 (3H, s), 2.5-2.8 (2H, m), 3.58 (2H, s), 3.7-3.9 (1H, m), 3.82 (3H, s), 3.91 (2H, s), 4.9-5.1 (1H, m), 5.29 (2H, s), 6.90 J = 8.6 Hz), 7.61 (1H, s), 7.66 (2H, d, J = 8.6 Hz). |
| 70 | 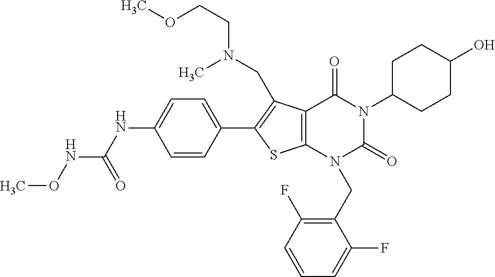 [SMILES: COCCN(C)Cc1c(-c2ccc(NC(=O)NOC)cc2)sc2c1c(=O)n(C1CCC(O)CC1)c(=O)n2Cc1c(F)cccc1F] | ¹H-NMR (CDCl₃) δ: 1.2-1.6 (3H, m), 1.6-1.8 (2H, m), 2.0- 2.1 (2H, m), 2.14 (3H, s), 2.5- 2.75 (4H, m), 3.31 (3H, s), 3.45 (2H, t, J = 5.8 Hz), 3.65- 3.85 (3H, m), 3.82 (3H, s), 4.9- 5.05 (1H, br), 5.30 (2H, s), 6.90 (2H, t, J = 8.0 Hz), 7.12 (1H, s), 7.25-7.4 (1H, m), 7.5- 7.6 (4H, m), 7.60 (1H, s). |
| 71 | 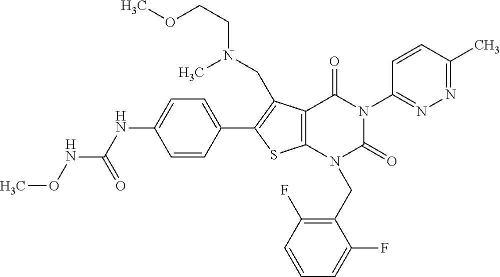 | ¹H-NMR (CDCl₃) δ: 2.13 (3H, s), 2.61 (2H, t, J = 5.8 Hz), 2.79 (3H, s), 3.26 (3H, s), 3.41 (2H, t, J = 5.8 Hz), 3.75-3.85 (2H, m), 3.82 (3H, s), 5.25-5.45 (2H, brm), 6.92 (2H, t, J = 8.2 Hz), 7.18 (1H, s), 7.2-7.7 (8H, m). |
| 72 | 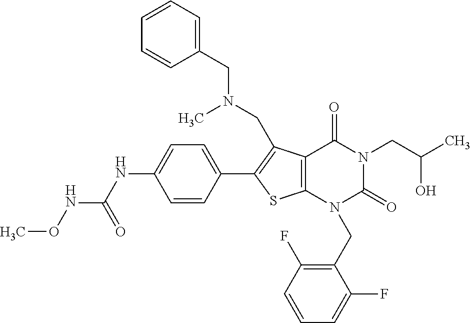 [SMILES: CONC(=O)Nc1ccc(-c2sc3c(c2CN(C)Cc2ccccc2)c(=O)n(CC(C)O)c(=O)n3Cc2c(F)cccc2F)cc1] | ¹H-NMR (CDCl₃) δ: 1.30 (3H, d, J = 6.0 Hz), 2.06 (3H, s), 2.90 (1H, d, J = 5.2 Hz), 3.57 (2H, s), 3.82 (3H, s), 3.91 (2H, s), 4.1- 4.25 (1H, m), 4.20 (2H, s), 5.34 (2H, s), 6.91 (2H, t, J = 8.0 Hz), 7.16 (1H, s), 7.2-7.4 (6H, m), 7.54 (2H, d, J = 8.8 Hz), 7.62 (1H, s), 7.67 (2H, d, J = 8.8 Hz). |
| 73 | 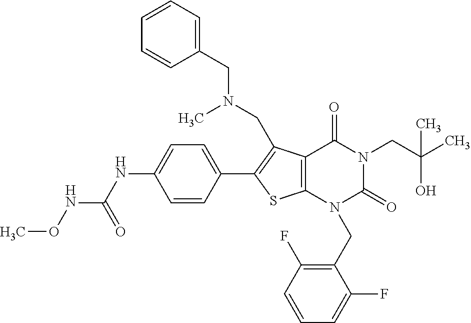 [SMILES: CONC(=O)Nc1ccc(-c2sc3c(c2CN(C)Cc2ccccc2)c(=O)n(CC(C)(C)O)c(=O)n3Cc2c(F)cccc2F)cc1] | ¹H-NMR (CDCl₃) δ: 1.29 (6H, s), 2.06 (3H, s), 3.56 (2H, s), 3.83 (3H, s), 3.91 (2H, s), 3.96 (1H, s), 4.28 (2H, s), 5.36 (2H, s), 6.91 (2H, t, J = 8.2 Hz), 7.13 (1H, s), 7.2-7.35 (6H, m), 7.54 (2H, d, J = 8.8 Hz), 7.62 (1H, s), 7.67 (2H, d, J = 8.8 Hz). |
| 74 | 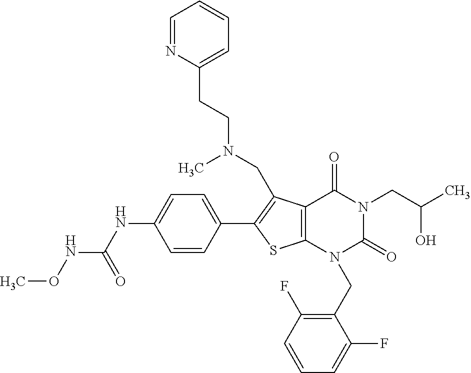 [SMILES: CONC(=O)Nc1ccc(-c2sc3c(c2CN(C)CCc2ccccn2)c(=O)n(CC(C)O)c(=O)n3Cc2c(F)cccc2F)cc1] | ¹H-NMR (CDCl₃) δ: 1.28 (3H, d, J = 5.8 Hz), 2.21 (3H, s), 2.75-2.95 (4H, m), 3.0-3.1 (1H m), 3.82 (5H, s), 4.1-4.2 (1H, m), 4.17 (2H, s), 5.34 (2H, s), 6.91 (2H, t, J = 8.2 Hz), 6.95- 7.1 (2H, m), 7.14 (1 H, s), 7.2- 7.4 (1H, m), 7.4-7.55 (5H, m), 7.59 (1H, s), 8.43 (1H, d, J = 5.0 Hz). |
| 75 | 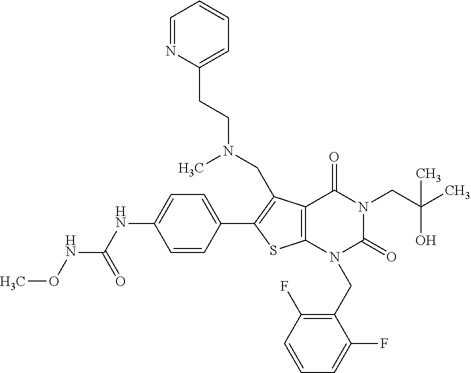 [SMILES: CONC(=O)Nc1ccc(-c2sc3c(c2CN(C)CCc2ccccn2)c(=O)n(CC(C)(C)O)c(=O)n3Cc2c(F)cccc2F)cc1] | ¹H-NMR (CDCl₃) δ: 1.26 (6H, s), 2.21 (3H, s), 2.75-2.95 (4H, m), 3.82 (5H, s), 3.99 (1H, s), 4.24 (2H, s), 5.36 (2H, s), 6.91 (2H, t, J = 8.2 Hz), 7.0-7.1 (2H, m), 7.13 (1H, s), 7.2-7.35 (1H, m), 7.45-7.55 (5H, m), 7.59 (1H, s), 8.43 (1H, d, J = 4.0 Hz). |
| 76 | 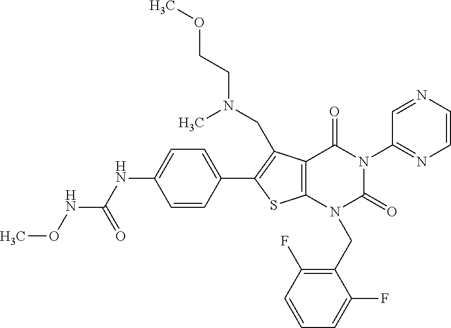 [SMILES: COCCN(C)Cc1c(-c2ccc(NC(=O)NOC)cc2)sc2c1c(=O)n(-c1cnccn1)c(=O)n2Cc1c(F)cccc1F] | ¹H-NMR (CDCl₃) δ: 2.13 (3H, s), 2.62 (2H, t, J = 6.0 Hz), 3.26 (3H, s), 3.41 (2H, t, J = 6.0 Hz), 3.79 (2H, s), 3.83 (1H, s), 5.36 (2H, s), 6.94 (2H, t, J = 8.0 Hz), 7.12 (1H, s), 7.2-7.4 (1H, m), 7.5-7.65 (5H, m), 8.65-8.7 (3H, m). |
| 77 | 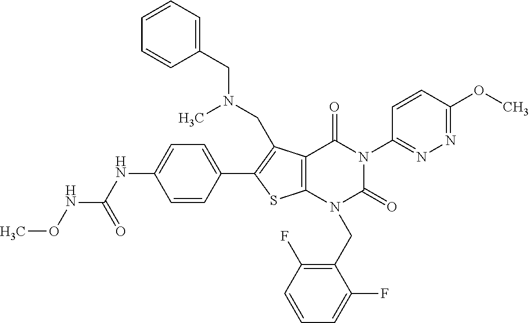 | ¹H-NMR (CDCl₃) δ: 2.06 (3H, s), 3.55 (2H, s), 3.83 (3H, s), 3.87 (2H, s), 4.19 (3H, s), 5.35 (2H, s), 6.92 (2H, t, J = 8.2 Hz), 7.1-7.45 (9H, m), 7.55 (2H, d, J = 8.4 Hz), 7.63 (1H, s), 7.72 (2H, d, J = 8.4 Hz). |
| 78 | 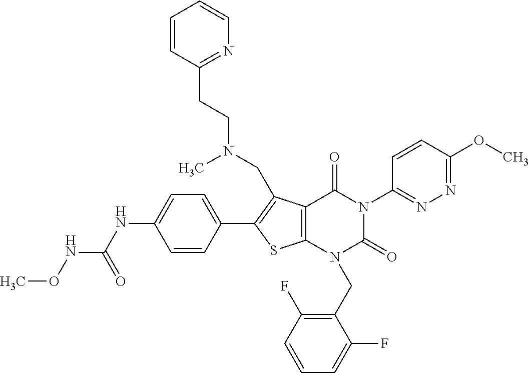 | ¹H-NMR (CDCl₃) δ: 2.20 (3H, s), 2.7-2.9 (4H, m), 3.78 (2H, s), 3.82 (3H, s), 4.19 (3H, s), 5.34 (2H, s), 6.85-7.2 (5H, m), 7.25-7.45 (2H, m), 7.45-7.7 (7H, m), 8.42 (1H, d, J = 4.0 Hz). |
| 79 | 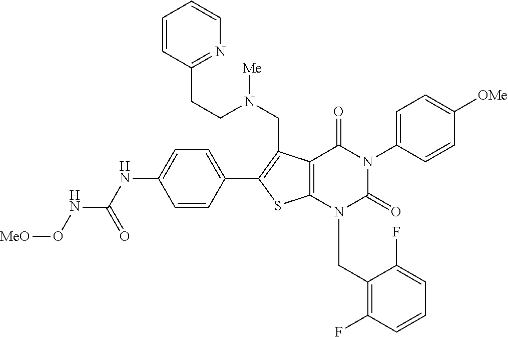 [SMILES: COONC(=O)Nc1ccc(-c2sc3c(c2CN(C)CCc2ccccn2)c(=O)n(-c2ccc(OC)cc2)c(=O)n3Cc2c(F)cccc2F)cc1] | ¹H NMR (CDCl₃) δ 2.20 (3H, s), 2.86 (4H, m), 3.82-3.84 (8H, m), 5.36 (2H, s), 6.92 (2H, d, J = 8.3 Hz), 7.00-7.06 (4H, m), 7.14-7.33 (4H, m), 7.46-7.51 (5H, m), 7.61 (1H, s), 8.42 (1H, d, J = 5.7 Hz). |
| 80 | 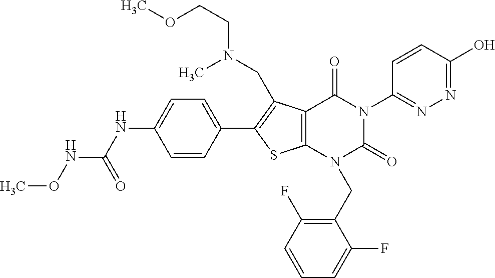 [SMILES: COCCN(C)Cc1c(-c2ccc(NC(=O)NOC)cc2)sc2c1c(=O)n(-c1ccc(O)nn1)c(=O)n2Cc1c(F)cccc1F] | ¹H-NMR (CDCl₃) δ: 2.12 (3H, s), 2.63 (2H, t, J = 5.8 Hz), 3.28 (3H, s), 3.43 (2H, t, J = 5.8 Hz), 3.79 (2H, s), 3.83 (3H, s), 5.35 (2H, s), 6.94 (2H, t, J = 8.2 Hz), 7.0-7.1 (1H, m), 7.2-7.4 (3H, m), 7.5-7.65 (4H, m), 7.63 (1H, s), 10.5-10.6 (1H, brs). |
| 81 | 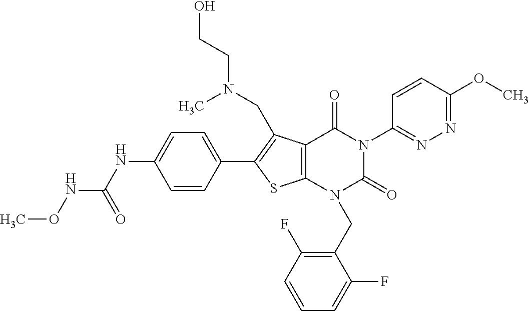 | ¹H-NMR (CDCl₃) δ: 1.98 (3H, s), 2.45-2.5 (2H, m), 2.9-3.2 (1H, m), 3.5-3.55 (2H, m), 3.65-3.85 (2H, brm), 3.82 (3H, s), 4.18 (3H, s), 5.34 (2H, s), 6.93 (2H, t, J = 8.0 Hz), 7.11 (1H, d, J = 9.0 Hz), 7.18 (1H, s), 7.25-7.35 (1H, m), 7.35-7.45 (3H, m), 7.57 (2H, d, J = 8.7 Hz), 7.66 (1H, s). |
| 82 | 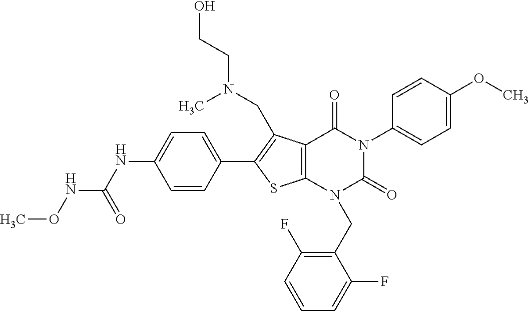 | ¹H-NMR (CDCl₃) δ: 1.99 (3H, s), 2.45-2.55 (2H, m), 3.5-3.6 (2H, m), 3.79 (2H, s), 3.82 (3H, s), 3.83 (3H, s), 5.36 (2H, s), 6.92 (2H, t, J = 8.0 Hz), 6.99 (2H, d, J = 8.8 Hz), 7.1-7.3 (4H, m), 7.39 (2H, d, J = 8.8 Hz), 7.56 (2H, d, J = 8.8 Hz), 7.64 (1H, s). |
| 83 | 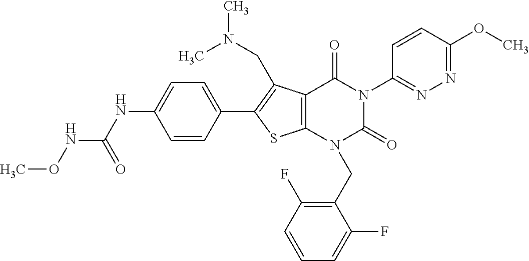 | ¹H-NMR (CDCl₃) δ: 2.15 (6H s), 3.6-3.8 (2H, m), 3.82 (3H, s), 4.18 (3H, s), 5.35 (2H), 6.92 (2H, t, J = 8.2 Hz), 7.12 (1H, d, J = 8.8 Hz), 7.2-7.65 (7H, m), 7.69 (1H, s). |
| 84 | 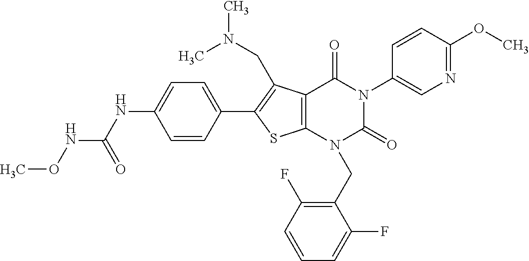 | ¹H-NMR (CDCl₃) δ: 2.13 (6H, s), 3.68 (2H, s), 3.83 (3H, s), 3.96 (3H, s), 5.36 (2H, s), 6.8- 7.0 (3H, m), 7.13 (1H, s), 7.2- 7.4 (1H, m), 7.45-7.65 (6H, m) 8.10 (1H, d, J = 2.6 Hz). |

### Propane-1,3-diones

Additional GnRH antagonists that may be used in conjunction with the compositions and methods described herein include optionally substituted propane-1,3-dione derivatives, such as (2R)—N-{5-[3-(2,5-difluorophenyl)-2-(1,3-dihydro-2H-benzimidazol-2-ylidene)-3-oxopropanoyl]-2-fluorobenzene-1-sulfonyl}-2-hydroxypropanimidamide, also referred to as opigolix or ASP-1707. Other GnRH antagonists of this chemical class that may be used for the treatment of estrogen-dependent diseases in accordance with the compositions and methods of the disclosure include compounds described in U.S. Pat. No. 6,960,591, the contents of which are incorporated herein by reference.

### Add-Back Therapy

Among the potential side-effects of GnRH antagonist therapy is a reduction in bone mineral density due to excessive depletion of estrogen (Newhall-Perry et al., American Journal of Obstetrics and Gynecology 173:824-829 (1995)). To combat this potential side effect, a patient undergoing GnRH antagonist therapy using the compositions and methods described herein can be administered add-back therapy. Add-back therapy may contain an estrogen (such as β17-estradiol, ethinyl estradiol, or a conjugated estrogen, such as a conjugated equine estrogen) optionally in combination with a progestin (such as norethindrone or an ester thereof, e.g., norethindrone acetate, or another agent such as progesterone, norgestimate, medroxyprogesterone, or drospirenone).

Endogenous estrogens are largely responsible for the development and maintenance of the female reproductive system and secondary sexual characteristics. Although circulating estrogens exist in a dynamic equilibrium of metabolic interconversions, estradiol is the principal intracellular human estrogen and is substantially more potent than its metabolites, estrone and estriol, at the receptor level. The primary source of estrogen in normally cycling adult women is the ovarian follicle, which secretes 70 to 500 μg of estradiol daily, depending on the phase of the menstrual cycle. After menopause, most endogenous estrogen is produced by conversion of androstenedione, secreted by the adrenal cortex, to estrone by peripheral tissues. Thus, estrone and the sulfate conjugated form, estrone sulfate, are the most abundant circulating estrogens in postmenopausal women. Circulating estrogens modulate the pituitary secretion of the gonadotropins, LH and FSH, through a negative feedback mechanism. Estrogens act to reduce the elevated levels of these hormones seen in postmenopausal women.

Progestin compounds, such as norethindrone and esters thereof (e.g., norethindrone acetate), as well as progesterone, norgestimate, medroxyprogesterone, and drospirenone, enhance cellular differentiation and generally oppose the actions of estrogens by decreasing estrogen receptor levels, increasing local metabolism of estrogens to less active metabolites, or inducing gene products that blunt cellular responses to estrogen. Progestins exert their effects in target cells by binding to specific progesterone receptors that interact with progesterone response elements in target genes. Progesterone receptors have been identified in the female reproductive tract, breast, pituitary, hypothalamus, and central nervous system. Progestins produce similar endometrial changes to those of the naturally occurring hormone progesterone. Progestins may be included in combination with estrogen in add-back therapy. For instance, according to the methods described herein, one can administer estrogen (e.g., E2) in conjunction with a progestin (e.g., norethindrone or an ester thereof, such as norethindrone acetate) to a patient undergoing GnRH antagonist therapy as to counteract the hypoestrogenemia that may be induced by the antagonist. In this way, add-back therapy can be used to mitigate or prevent potentially deleterious side effects, such as a reduction in bone mineral density.

Add-back therapy may be formulated for oral administration. For instance, add-back therapy administered in conjunction with the compositions and methods described herein may be formulated as a tablet, capsule, gel cap, powder, liquid solution, or liquid suspension. In some embodiments, the add-back therapy includes both an estrogen, such as β17-estradiol, and a progestin, such as norethindrone or norethindrone acetate. The estrogen and progestin may be administered separately or admixed in a single composition, such as a single tablet, capsule, gel cap, powder, liquid solution, or liquid suspension. For example, add-back therapy may feature a co-formulation containing estrogen (e.g., in the form of E2) and an additional agent such as a progestin (e.g., norethindrone or a compound that is metabolized in vivo to produce norethindrone, such as an ester of norethindrone that is de-esterified in vivo to produce norethindrone, for instance, norethindrone acetate). In some embodiments, add-back therapy is administered to a patient in the form of a single tablet, capsule, gel cap, powder, liquid solution, or liquid suspension that contains both estrogen (e.g., in the form of E2) and a progestin (e.g., norethindrone or a compound that is metabolized in vivo to produce norethindrone, such as an ester of norethindrone that is de-esterified in vivo to produce norethindrone, for instance, norethindrone acetate). In some embodiments, add-back therapy is administered as a fixed dose combination containing a GnRH antagonist, estrogen, and one or more additional agents, such as a progestin, in a single pharmaceutical composition. For instance, add-back therapy may be administered as a fixed dose combination of a GnRH antagonist, estrogen (e.g., in the form of E2) and a progestin (e.g., norethindrone or a compound that is metabolized in vivo to produce norethindrone, such as an ester of norethindrone that is de-esterified in vivo to produce norethindrone, for instance, norethindrone acetate) in the form of a single pharmaceutical composition, such as a single tablet, capsule, gel cap, powder, liquid solution, or liquid suspension.

Despite the utility of add-back therapy, periodic administration of an estrogen and/or progestin can be associated with adverse side effects (see, for example, Activella® Physician Insert, Novo Nordisk Inc. (Princeton, N.J.), December 2006, the disclosure of which is incorporated herein by reference in its entirety. The present disclosure is based, in part, on the surprising and advantageous discovery that add-back therapy can be safely administered to patients, such as patients suffering from an estrogen-dependent disease described herein, over the course of extended treatment periods when administered in combination with a GnRH antagonist (particularly a thieno[3,4d]pyrimidine compound, such as 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid or a pharmaceutically acceptable salt thereof).

### Effects of GnRH Antagonist Therapy on Serum Lipids

The present disclosure is based, at least in part, on the discovery that GnRH antagonists, particularly thieno[3,4d]pyrimidine compounds, such as 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid or a pharmaceutically acceptable salt thereof (e.g., the choline salt thereof) can be administered to a patient having an estrogen-dependent disease over extended periods of time without inducing a significant increase in serum lipid levels. The compositions and methods of the disclosure thus provide the benefit of being able to alleviate the symptoms and the underlying pathology of an estrogen-dependent disease without causing an elevation in total cholesterol and low-density lipoprotein-cholesterol.

In addition, the compositions and methods of the disclosure can also be used to monitor a patient's concentration one or more endogenous serum lipids over the course of a treatment period during which the patient is periodically administered a GnRH antagonist. For example, in some embodiments of the disclosure, a method is provided in which a patient is periodically treated with a GnRH antagonist over the course of a treatment period (e.g., a treatment period having a duration of at least 1 year) so as to treat, or alleviate the symptoms of, an estrogen-dependent disease, such as endometriosis, uterine fibroids, adenomyosis, or rectovaginal endometriosis. In some embodiments, the method includes monitoring the patient's level of total cholesterol during the treatment period. The method may further include determining that the patient does not exhibit an increase (e.g., a significant increase) in total cholesterol relative to a measurement of the patient's total cholesterol obtained prior to the treatment period, and continuing to administer (e.g., periodically) the GnRH antagonist to the patient during the treatment period (e.g., for the remainder of the treatment period). For example, the method may include monitoring the patient's level of total cholesterol after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in total cholesterol (e.g., of greater than 20% (e.g., determining that the patient does not exhibit an increase in total cholesterol of greater than 20%, 19%, 18%, 17%, 16%, 15%, 14%, 13%, 12%, 11%, 10%, 9%, 8%, 7%, 6%, 5%, 4%, 3%, 2%, or 1%)) relative to a measurement of the patient's total cholesterol obtained prior to the treatment period, and continuing to administer (e.g., periodically) the GnRH antagonist to the patient during the treatment period (e.g., for the remainder of the treatment period).

In some embodiments, the method includes monitoring the patient's low-density lipoprotein-cholesterol level during the treatment period. The method may further include determining that the patient does not exhibit an increase (e.g., a significant increase) in low-density lipoprotein-cholesterol relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period). For example, the method may include monitoring the patient's low-density lipoprotein-cholesterol level after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in low-density lipoprotein-cholesterol level (e.g., of greater than 40% (e.g., determining that the patient does not exhibit an increase in low-density lipoprotein-cholesterol level of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%)) relative to a measurement of the patient's low-density lipoprotein-cholesterol level obtained prior to the treatment period, and continuing to administer (e.g., periodically) the GnRH antagonist to the patient during the treatment period (e.g., for the remainder of the treatment period).

In some embodiments, the method includes monitoring the patient's low-density lipoprotein-cholesterol level after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in low-density lipoprotein-cholesterol level to a level greater than 160 mg/dl (e.g., determining that the patient does not exhibit an increase in low-density lipoprotein-cholesterol level to a level greater than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl), and continuing to administer (e.g., periodically) the GnRH antagonist to the patient during the treatment period (e.g., for the remainder of the treatment period).

In some embodiments, the method includes monitoring the patient's low-density lipoprotein-cholesterol level after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in low-density lipoprotein-cholesterol level from a level less than 160 mg/dl (e.g., from a level less than 160 mg/dl, 159 mg/dl, 158 mg/dl, 157 mg/dl, 156 mg/dl, 155 mg/dl, 154 mg/dl, 153 mg/dl, 152 mg/dl, 151 mg/dl, 150 mg/dl, 149 mg/dl, 148 mg/dl, 147 mg/dl, 146 mg/dl, 145 mg/dl, 144 mg/dl, 143 mg/dl, 142 mg/dl, 141 mg/dl, 140 mg/dl, 139 mg/dl, 138 mg/dl, 137 mg/dl, 136 mg/dl, 135 mg/dl, 134 mg/dl, 133 mg/dl, 132 mg/dl, 131 mg/dl, or 130 mg/dl) to a level greater than 190 mg/dl, and continuing to administer (e.g., periodically) the GnRH antagonist to the patient during the treatment period (e.g., for the remainder of the treatment period).

In some embodiments, the method includes monitoring the patient's ratio of low-density lipoprotein-cholesterol to high-density lipoprotein-cholesterol during the treatment period. The method may further include determining that the patient does not exhibit an increase (e.g., a significant increase) in their ratio of low-density lipoprotein-cholesterol to high-density lipoprotein-cholesterol relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period). In some embodiments, the method includes monitoring the patient's ratio of low-density lipoprotein-cholesterol to high-density lipoprotein-cholesterol after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in their ratio of low-density lipoprotein-cholesterol to high-density lipoprotein-cholesterol (e.g., of greater than 40% (e.g., determining that the patient does not exhibit an increase in their ratio of low-density lipoprotein-cholesterol to high-density lipoprotein-cholesterol of greater than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%)) relative to a measurement of their ratio of low-density lipoprotein-cholesterol to high-density lipoprotein-cholesterol obtained prior to the treatment period, and continuing to administer (e.g., periodically) the GnRH antagonist to the patient during the treatment period (e.g., for the remainder of the treatment period).

In some embodiments, the method includes monitoring the patient's serum triglyceride level during the treatment period. The method may further include determining that the patient does not exhibit an increase (e.g., a significant increase) in serum triglyceride level relative to a measurement of the patient's triglyceride level obtained prior to the treatment period, and continuing to administer (e.g., periodically) the GnRH antagonist to the patient during the treatment period (e.g., for the remainder of the treatment period). In some embodiments, the method includes monitoring the patient's serum triglyceride level after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in serum triglyceride level (e.g., of greater than 50% (e.g., determining that the patient does not exhibit an increase in serum triglyceride level of greater than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%)) relative to a measurement of the patient's triglyceride level obtained prior to the treatment period, and continuing to administer (e.g., periodically) the GnRH antagonist to the patient during the treatment period (e.g., for the remainder of the treatment period).

In some embodiments, the method includes monitoring the patient's serum triglyceride level during the treatment period, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period). In some embodiments, the method includes monitoring the patient's serum triglyceride level after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in serum triglyceride level to a level greater than 200 mg/dl, and continuing to administer (e.g., periodically) the GnRH antagonist to the patient during the treatment period (e.g., for the remainder of the treatment period).

In some embodiments, the method includes monitoring the patient's serum triglyceride level during the treatment period, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period). In some embodiments, the method includes monitoring the patient's serum triglyceride level after from about 6 weeks to about 52 weeks (e.g., after about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks) of treatment with the GnRH antagonist, determining that the patient does not exhibit an increase in serum triglyceride level from a level less than 300 mg/dl to a level greater than 500 mg/dl, and continuing to administer (e.g., periodically) the GnRH antagonist to the patient during the treatment period (e.g., for the remainder of the treatment period).

Assays that can be used to assess the level of total cholesterol, low-density lipoprotein-cholesterol, high-density lipoprotein-cholesterol, and/or triglycerides in a patient (e.g., in a sample, such as a blood serum sample, obtained from the patient are described, for example, in U.S. Pat. Nos. 9,051,599; 5,814,472; and 5,215,886, the disclosures of each of which are incorporated herein by reference as they pertain to compositions and methods for lipid detection.

### Methods of Treating Estrogen-Dependent Diseases

Using the compositions and methods described herein, a patient having an estrogen-dependent diseases, such as uterine fibroids, endometriosis (e.g., rectovaginal endometriosis), and/or adenomyosis, among others, may be administered a GnRH antagonist as described herein. Additional examples of estrogen-dependent diseases that may be treated using the compositions and methods of the disclosure include, without limitation, benign prostatic hypertrophy, precocious puberty, premenstrual syndrome, polycystic ovary syndrome, hirsutism, short stature, a sleep disorder, acne, baldness, and irritable bowel syndrome.

A variety of methods known in the art and described herein can be used to determine whether a patient is responding favorably to GnRH antagonist treatment. For instance, beneficial clinical results in response to GnRH antagonist therapy include, without limitation, alleviation of symptoms of the endometrial growth disorder. Indications of successful treatment of a uterine fibroids patient include, for example, (i) a reduction in serum concentration of FSH, LH, and/or E2 following administration of the GnRH antagonist to the patient, (ii) a reduction in uterine blood loss following administration of the GnRH antagonist to the patient, (iii) elimination of heavy menstrual bleeding following administration of the GnRH antagonist to the patient, and (iv) induction of amenorrhea in the patient following administration of the GnRH antagonist to the patient.

Similarly, clinical indicators of successful treatment of an endometriosis patient (e.g., a rectovaginal endometriosis patient) that is administered a GnRH antagonist include, without limitation, (i) a reduction in serum concentration of FSH, LH, and/or E2 following administration of the GnRH antagonist to the patient, (ii) a reduction in the volume of one or more endometriosis nodes (e.g., rectovaginal endometriosis nodes) following administration of the GnRH antagonist to the patient, (iii) a reduction in bowel involvement of one or more type III endometriosis nodes following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine bleeding following administration of the GnRH antagonist to the patient; (ix) achievement of amenorrhea following administration of the GnRH antagonist to the patient; and (x) an improvement in the patient's overall well-being as determined by an improvement in the patient's Endometriosis Health Profile questionnaire (EHP-30) score following administration of the GnRH antagonist to the patient and/or by way of a positive Patient Global Impression of Change (PGIC) score following administration of the GnRH antagonist to the patient.

Exemplary indicia of successful treatment of an adenomyosis patient that is administered a gonadotropin-releasing hormone (GnRH) antagonist include, without limitation, (i) a reduction in serum concentration of FSH, LH, and/or E2 following administration of the GnRH antagonist to the patient, (ii) a reduction in uterine volume following administration of the GnRH antagonist to the patient, (iii) a reduction in the thickness of the anterior and/or posterior region of the uterine myometrium following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine tenderness following administration of the GnRH antagonist to the patient; (ix) a reduction in uterine bleeding following administration of the GnRH antagonist to the patient; (x) achievement of amenorrhea following administration of the GnRH antagonist to the patient; (xi) a reduction in the diameter of a junctional zone of adenomyosis following administration of the GnRH antagonist to the patient; and (xii) an improvement in the patient's overall well-being as determined by an improvement in the patient's EHP-30 score following administration of the GnRH antagonist to the patient and/or by way of a positive PGIC score following administration of the GnRH antagonist to the patient.

### Modified Biberoglu and Behrman Symptom Severity Scale

Exemplary methods for assessing a patient's response to GnRH antagonist therapy for the treatment of estrogen-dependent diseases, such as uterine fibroids, endometriosis (e.g., rectovaginal endometriosis) and/or adenomyosis, among others described herein, include administration of a modified Biberoglu and Behrman questionnaire, as described herein. An exemplary mB&B questionnaire for use in conjunction with the compositions and methods described herein is shown in Table 4, below.

### Endometriosis Health Profile Questionnaire

Additional methods for assessing patient respond to GnRH antagonist therapy for the treatment of estrogen-dependent diseases, such as uterine fibroids, endometriosis (e.g., rectovaginal endometriosis) and/or adenomyosis, among others described herein, include analyzing the patient's score on an Endometriosis Health Profile questionnaire. An exemplary Endometriosis Health Profile questionnaire for use in conjunction with the compositions and methods described herein is the EHP-30 questionnaire shown in Table 5, below.

### Patient Global Impression of Change Score

Additional methods for assessing patient response to GnRH antagonist therapy for the treatment of an estrogen-dependent disease described herein, such as uterine fibroids, endometriosis (e.g., rectovaginal endometriosis, and/or adenomyosis, among others) include analyzing the patient's score on a Patient Global Impression of Change (PGIC) scale. An exemplary PGIC questionnaire for use in conjunction with the compositions and methods described herein is shown in Table 6, below.

### Quantitation of Uterine Blood Loss by the Alkaline Hematin Method

Techniques for quantifying uterine blood loss are known in the art and include, for instance, the alkaline hematin method, as described, for instance, in Hallberg et al., Scand. J. Clin. Lab. Invest. 16:244-248 (1964), the disclosure of which is incorporated herein by reference as it pertains to techniques for assessing the volume of blood lost by a patient. In the alkaline hematin approach, uterine blood soaked into, for example, a sanitary napkin, vaginal tampon, or cotton pad, is reconstituted in a basic aqueous solution, such as a solution of 5% (w/v) sodium hydroxide. This incubation enables (i) extraction of the iron-containing porphyrin of hemoglobin and (ii) oxidation of the ferrous ion to a hydroxy-coordinated ferric ion in each chelate, thus forming hematin. Hematin is a detectable chromophore, absorbing light at between 550 and 546 nm. By comparing the concentration of hematin obtained from incubation of a soaked menstrual blood sample with aqueous sodium hydroxide to the concentration of hematin obtained from incubation of a sample of venous blood with aqueous sodium hydroxide, one can stoichiometrically determine the volume of menstrual blood lost by a patient, such as a patient having an estrogen-dependent disease. Improvements to the original alkaline hematin method are known in the art and are described, for example, in Newton et al., Contraception 16:269-282 (1977), and in van Eijkeren et al., Eur. J. Obstet. Gynecol. Reprod. Biol. 22:345-351 (1986), the disclosures of each of which are incorporated herein by reference as they pertain to methods of determining the volume of blood lost by a patient.

### Routes of Administration and Dosing of GnRH Antagonists

The GnRH antagonists described herein may be administered to a patient in need thereof (e.g., a patient suffering from an estrogen-dependent disease, such as uterine fibroids, endometriosis (e.g., rectovaginal endometriosis) and/or adenomyosis, among others described herein) by a variety of routes of administration. For instance, the GnRH antagonists described herein may be formulated for oral administration, among other routes. Exemplary non-oral routes of administration of the GnRH antagonists described herein include, without limitation, intravenous administration, parenteral administration, subcutaneous administration, intramuscular administration, and intradermal administration, among others.

In some embodiments, the GnRH antagonist is a compound of any one of formulas (I)-(VIa), above, and is administered to the patient in an amount of from about 25 mg to about 500 mg per dose, and may be administered in one or more doses per day, for example, in accordance with a dosing schedule described above. For instance, the GnRH antagonist may a compound of any one of formulas (I)-(VIa), above, and may be administered to the patient in an amount of from 25 mg to 500 mg, 30 mg to 495 mg, 35 mg to 490 mg, 40 mg to 485 mg, 45 mg to 480 mg, 50 mg to 475 mg, 55 mg to 470 mg, 60 mg to 465 mg, 65 mg to 460 mg, 70 mg to 455 mg, 75 mg to 450 mg, 80 mg to 445 mg, 85 mg to 440 mg, 90 mg to 435 mg, 95 mg to 430 mg, 100 mg to 425 mg, 105 mg to 420 mg, 110 mg to 415 mg, 115 mg to 410 mg, 120 mg to 405 mg, 125 mg to 400 mg, 130 mg to 395 mg, 135 mg to 390 mg, 140 mg to 385 mg, 145 mg to 380 mg, or 150 mg to 375 mg, per dose, and may be administered in one or more doses per day (e.g., in a single daily dose). In some embodiments, the GnRH antagonist is a compound of any one of formulas (I)-(VIa), above, and is administered to the patient once daily in an amount of from 100 mg to 300 mg, per dose, such as from 105 mg to 295 mg, 110 mg to 290 mg, 115 mg to 285 mg, 120 mg to 280 mg, 125 mg to 275 mg, 130 mg to 270 mg, 135 mg to 265 mg, 140 mg to 260 mg, 145 mg to 255 mg, 150 mg to 250 mg, 155 mg to 245 mg, 160 mg to 240 mg, 165 mg to 235 mg, 170 mg to 230 mg, 175 mg to 225 mg, 180 mg to 220 mg, 185 mg to 215 mg, 190 mg to 210 mg, or 195 mg to 205 mg, per dose. In some embodiments, the GnRH antagonist is a compound of any one of formulas (I)-(VIa), above, and is administered to the patient once daily in an amount of about 50 mg, 75 mg, 100 mg, or 200 mg per dose.

The GnRH antagonists described herein may be administered to a patient a plurality of times over the course of a treatment period, such as a treatment period of at least 52 weeks, as described herein.

### Pharmaceutical Compositions

GnRH antagonists suitable for use with the compositions and methods described herein can be formulated into a pharmaceutical composition for administration to a patient, such as a female human patient, in a biologically compatible form suitable for administration in vivo. A pharmaceutical composition containing a GnRH antagonist, such as a compound described herein (e.g., 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid or a pharmaceutically acceptable salt thereof, such as the choline salt thereof), may additionally contain a suitable diluent, carrier, or excipient. GnRH antagonists can be administered to a patient, for example, orally or by intravenous injection. Under ordinary conditions of storage and use, a pharmaceutical composition may contain a preservative, e.g., to prevent the growth of microorganisms. Conventional procedures and ingredients for the selection and preparation of suitable formulations are described, for example, in Remington: The Science and Practice of Pharmacy (2012, 22^{nd} ed.) and in The United States Pharmacopeia: The National Formulary (2015, USP 38 NF 33).

Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a patient in need of treatment.

A pharmaceutical composition may be administered to a patient, e.g., a human patient, alone or in combination with one or more pharmaceutically acceptable carriers, e.g., as described herein, the proportion of which may be determined by the solubility and/or chemical nature of the compound, chosen route of administration, and standard pharmaceutical practice.

### Compound for Use

In another aspect, the disclosure provides a GnRH antagonist (e.g., a GnRH antagonist described herein) for use in any of the methods described herein. For example, the disclosure features a GnRH antagonist, such as a GnRH antagonist described herein, for use in a method of treating an estrogen-dependent disease, such as uterine fibroids, endometriosis, adenomyosis, or rectovaginal endometriosis. The method may feature, for example, any one or more of the method steps recited herein.

### Medicament

In another aspect, the disclosure provides a GnRH antagonist (e.g., a GnRH antagonist described herein) for use in the manufacture of a medicament for performing any of the methods described herein. For example, the disclosure features a GnRH antagonist, such as a GnRH antagonist described herein, for use in the manufacture of a medicament for use in a method of treating an estrogen-dependent disease, such as uterine fibroids, endometriosis, adenomyosis, or rectovaginal endometriosis. The method may feature, for example, any one or more of the method steps recited herein.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regards as their invention.

### Example 1. GnRH Antagonist Therapy Alleviates Estrogen-Dependent Disease Symptoms without Inducing Significant Bone Mineral Density Loss, Even when Administered Over an Extended Treatment Period

### Objective

Endometriosis, an estrogen-dependent gynecological condition defined as the presence of endometrial-like tissue outside the uterus, is one of the most common gynecological diseases, affecting about 1 in 10 women during their reproductive years. A chronic, inflammatory reaction, induced by the ectopic endometrial cells, results in a variety of pain symptoms including dysmenorrhea (DYS), non-menstrual pelvic pain (NMPP), overall pelvic pain (OPP), dyspareunia, and dyschezia. Complete suppression of estrogen (E2) can reduce estrogen-associated pain (EAP), but may also result in substantial bone mineral density (BMD) loss. The purpose of the experiments described in this example was to evaluate the effects of prolonged GnRH antagonist treatment on patients suffering from an estrogen-dependent disease, such as endometriosis.

### Methods

This example describes the results of a Phase 2b, double-blind, randomized, placebo-controlled, multicenter, human clinical trial undertaken to evaluate the efficacy of a GnRH antagonist represented by formula (VI) in a series of patients suffering from an estrogen-dependent disease. In this trial, a series of human female patients diagnosed as having endometriosis were administered the choline salt of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid periodically over the course of a 52-week treatment period. After 52 weeks of treatment, the patients were monitored for severity of disease symptoms and bone mineral density.

Participants were women aged 18 to 45 years with surgically confirmed endometriosis and moderate to severe EAP. Women with a history of osteoporosis or other metabolic bone disease were excluded. Certain subjects completing an initial 24-week treatment period were invited to continue in a treatment extension phase, resulting in a total of up to 52 weeks of treatment. Subjects that completed 52 weeks of treatment constituted the treatment extension analysis set (TEAS) of patients.

Patients were randomized to either (i) 50 mg/day, 75, mg/day, 100 mg/day, or 200 mg/day of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid, administered as a choline salt, or (ii) matching placebo. Patients assigned to the placebo cohort were switched to 100 mg/day of 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid at week 12.

Patients that received 75 mg/day of compound (VI) were divided into two groups: those that received a fixed dose (FD) of compound (VI) at 75 mg/day throughout the 52-week treatment period, and those that received a titrated dose (TD) of 75 mg/day of compound (VI) for the first 12 weeks of the treatment period, followed by a daily dose of either 50 mg, 75 mg, or 100 mg of the compound depending on each patient's serum E2 concentration after 4 weeks or 8 weeks of treatment. Dosages from week 12 onward for patients in the 75 mg/day TD cohort were determined according to the following schedule:

TABLE 7
Dosing schedule for patients in the 75 mg/day TD cohort

| | Mean Serum E2 | |
|---|---|---|
| Initial Daily | Concentration | New Daily Dosage of |
| Dosage of | at Week 4 or 8 of the | Compound (VI) from |
| Compound (VI) | Treatment Period | Week 12 Onward |
| 75 mg/day | <20 pg/ml | 50 mg/day |
| 75 mg/day | 20-50 pg/ml | 75 mg/day |
| 75 mg/day | >50 pg/ml | 100 mg/day |

Patients that were treated with 200 mg/day of the compound at the 24-week timepoint in the treatment period were switched to 100 mg/day for the final 24 weeks of the study.

EAP was assessed using questionnaires in a daily electronic diary. Mean 28-day scores for pelvic pain were calculated at each month up to 12 months and compared to baseline. Efficacy was assessed as the proportion of patients who had at least 30% reduction in OPP at 12 weeks. DYS, NMPP, dyspareunia, and dyschezia were also assessed.

Quality of life endpoints included a measurement of each patient's difficulty of doing daily activities, Patient Global Impression of Change (PGIC), and Endometriosis Health Profile-30 (EHP-30) score.

Another goal of this study was to monitor changes in patients' levels of total cholesterol, low-density lipoprotein-cholesterol (LDL-C), high-density lipoprotein-cholesterol (HDL-C), and serum triglycerides, as well as patients' ratios of low-density lipoprotein-cholesterol to high-density lipoprotein-cholesterol (LDL-C/HDL-C) throughout the duration of the treatment period. Accordingly, patients were evaluated for changes in the foregoing serum lipids at various timepoints throughout the course of the study. Patients were not required to be fasted for serum lipid analysis except at baseline.

Patients were not required to take calcium or vitamin D supplements. Patients were allowed to use analgesics for treatment of pelvic pain.

BMD of the femoral neck, total hip and lumbar spine was assessed by DXA at baseline and after 12 and 24 weeks of treatment. Reading of DXA scans and monitoring of scan quality, including cross-calibration between sites, was centralized.

### Results

A series of n=327 human female patients having clinically-diagnosed endometriosis were randomly assigned to, and treated in, cohorts in which patients were administered placebo or the GnRH antagonist in a once-daily amount of 50 mg/day, 100 mg/day, 75 mg/day (FD or TD), or 200 mg/day. Of these, 253 subjects (77.1%) completed the initial 24-week treatment, of which 176 subjects participated in the treatment extension phase, resulting in a total of 52 weeks of continuous treatment. 108 subjects (60.0%) completed the full treatment period of 52 weeks.

Baseline characteristics of randomized and treated subjects are shown in Table 8, below.

TABLE 8
Demographics and baseline characteristics

| | Placebo | 50 mg | 75 mg FD | 75 mg TD | 100 mg | 200 mg |
|---|---|---|---|---|---|---|
| | (N = 53) | (N = 49) | (N = 56) | (N = 58) | (N = 51) | (N = 56) |
| Median age- | 32 (22-45) | 31 (22-43) | 33 (18-45) | 31.5 (21-45) | 33 (22-44) | 31 (19-45) |
| years (range) | | | | | | |
| Mean BMI- | 27 (7.6) | 26 (7.7) | 26 (6.0) | 27 (7.2) | 26 (6.0) | 25 (5.5) |
| kg/m² (SD) | | | | | | |
| Race: % White, | 92.5, 7.5 | 95.9, 4.1 | 91.1, 10.7 | 86.2, 13.8 | 94.1, 7.8 | 94.6, 7.1 |
| % Black | | | | | | |
| Mean OPP score | 1.8 (0.46) | 1.7 (0.49) | 1.7 (0.47) | 1.7 (0.49) | 1.8 (0.46) | 1.7 (0.51) |
| (0-3 VRS) (SD) | | | | | | |
| Mean DYS score | 2.1 (0.44) | 2.1 (0.39) | 2.1 (0.43) | 2.1 (0.45) | 2.1 (0.40) | 2.1 (0.42) |
| (0-3 VRS) (SD) | | | | | | |
| Mean NMPP | 1.7 (0.50) | 1.5 (0.56) | 1.6 (0.53) | 1.5 (0.56) | 1.7 (0.50) | 1.6 (0.58) |
| score (0-3 VRS) | | | | | | |
| (SD) | | | | | | |
| Mean | 1.7 (0.80) | 1.4 (0.82) | 1.7 (0.86) | | 1.7 (0.89) | 1.6 (0.84) |
| dyspareunia | | | | | | |
| score (0-3 VRS) | | | | | | |
| (SD) | | | | | | |
| Mean dyschezia | 5.9 (2.40) | 4.6 (2.84) | 5.8 (2.51) | | 5.9 (2.65) | 5.4 (2.56) |
| score (0-10 NRS) | | | | | | |
| (SD) | | | | | | |
| Mean HDL-C | 59 (15) | 67 (19) | 67 (19) | 66 (15) | 64 (15) | 65 (15) |
| level (mg/dl) (SD) | | | | | | |
| Mean LDL-C | 109 (31) | 103 (24) | 107 (30) | 106 (29) | 104 (25) | 102 (22) |
| level (mg/dl) (SD) | | | | | | |
| Mean | 105 (64) | 101 (97) | 96 (47) | 105 (67) | 107 (72) | 96 (60) |
| triglyceride level | | | | | | |
| (mg/dl) (SD) | | | | | | |
| Ratio of mean | 2.0 (0.82) | 1.7 (0.68) | 1.8 (0.74) | 1.7 (0.76) | 1.7 (0.59) | 1.6 (0.51) |
| LDL-C level to | | | | | | |
| mean HDL-C | | | | | | |
| level | | | | | | |

Therapeutic Effects of GnRH Antagonist Treatment

After 52 weeks of continuous, once-daily treatment, administration of the GnRH antagonist ceased. Patients treated with 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid exhibited a sustained reductions in overall pelvic pain, dysmenorrhea, non-menstrual pelvic pain, dyspareunia, and dyschezia, as well as improvements in quality of life. For example, GnRH antagonist-treated patients exhibited an overall reduction in overall pelvic pain, as shown in Table 9, below, for patients treated with 75 mg/day or 200 mg/day of the GnRH antagonist. This table depicts the proportion of patients that exhibited an overall pelvic pain score reduction of greater than 30% from baseline using a VRS scale following the cessation of GnRH antagonist treatment.

TABLE 9
Overall Pelvic Pain Responder Rates (Defined as
patients exhibiting an overall pelvic pain VRS
score reduction of >30% from baseline VRS score)
Overall Pelvic Pain Responder Rate (%)

| Dose (Once-daily) | 75 mg | 200 mg |
|---|---|---|
| After 52 weeks of GnRH | 69.2% | 82.4% |
| antagonist treatment | | |

In addition to exhibiting a sustained reduction in disease symptomology, patients that were treated with the GnRH antagonist over the full 52-week treatment period did not exhibit a significant decrease in bone mineral density. Table 10, below, depicts the mean change in bone mineral density from a baseline, pre-treatment measurement following completion of the 52-week treatment period for patients treated with 75 mg/day or 200 mg/day of the GnRH antagonist. All bone mineral density measurements shown below were obtained by assessing bone mineral density at each patient's spine.

TABLE 10
Bone mineral density following 52-week treatment period
Bone Mineral Density (Mean % change from baseline)

| Dose (Once-daily) | 75 mg | 200 mg |
|---|---|---|
| After 52 weeks of GnRH | −1.139% | −2.188% |
| antagonist treatment | | |

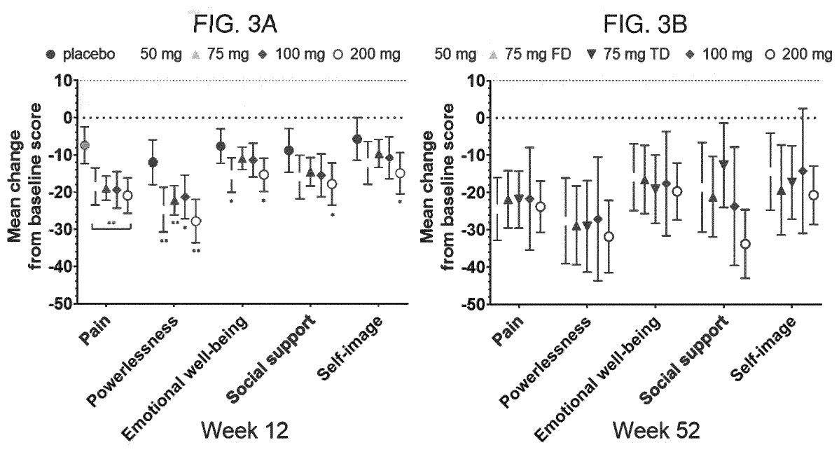

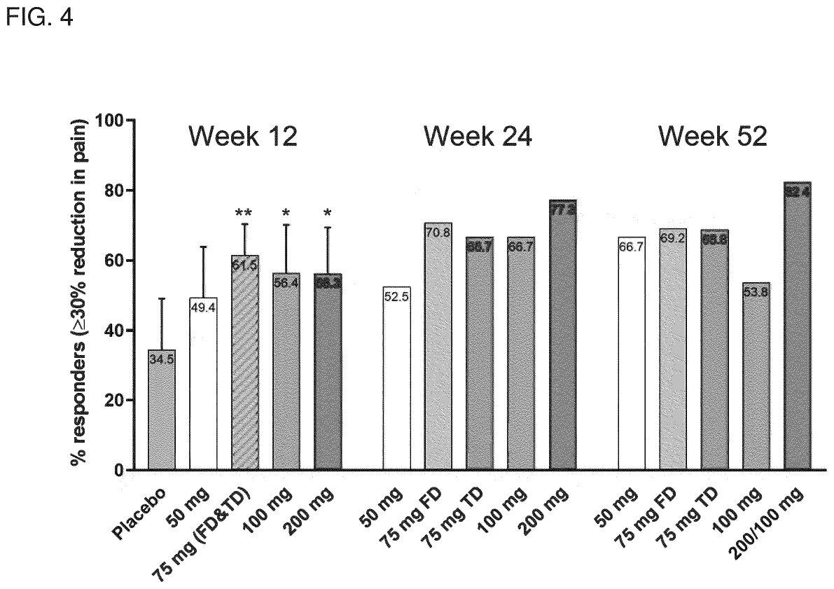

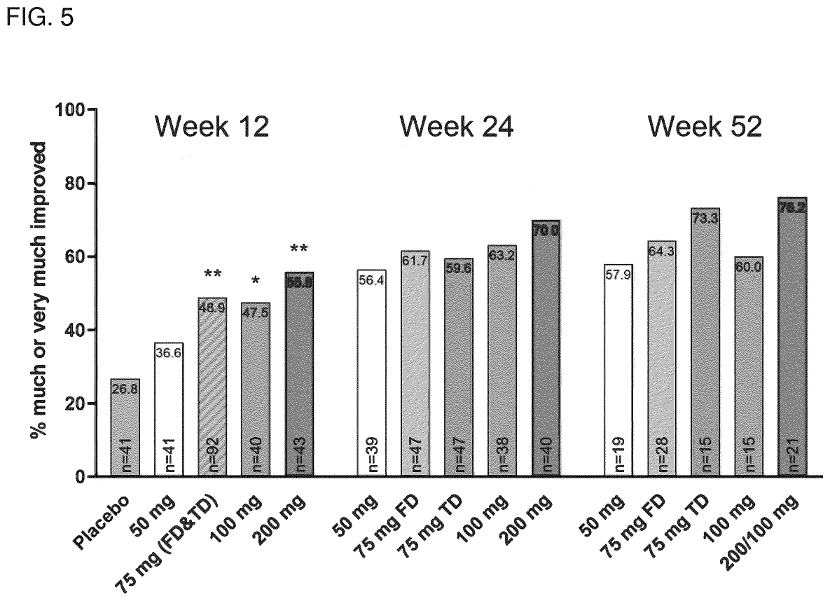

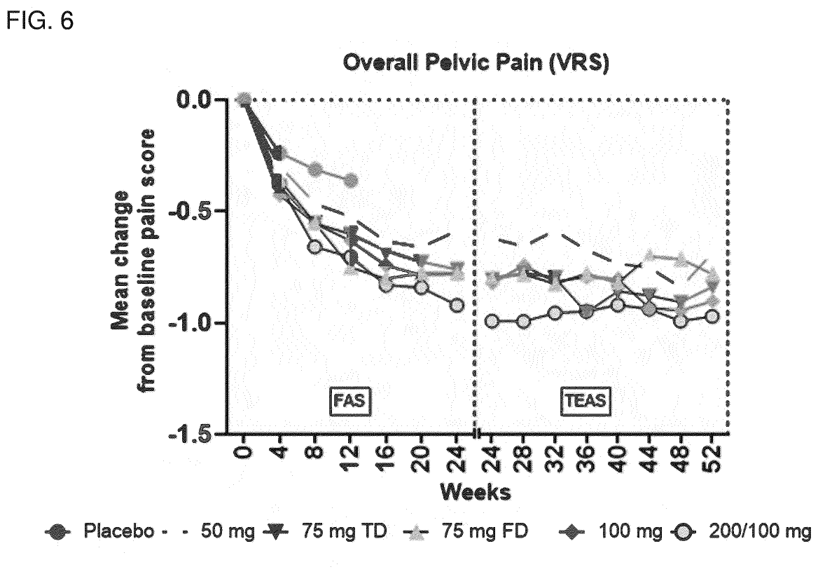

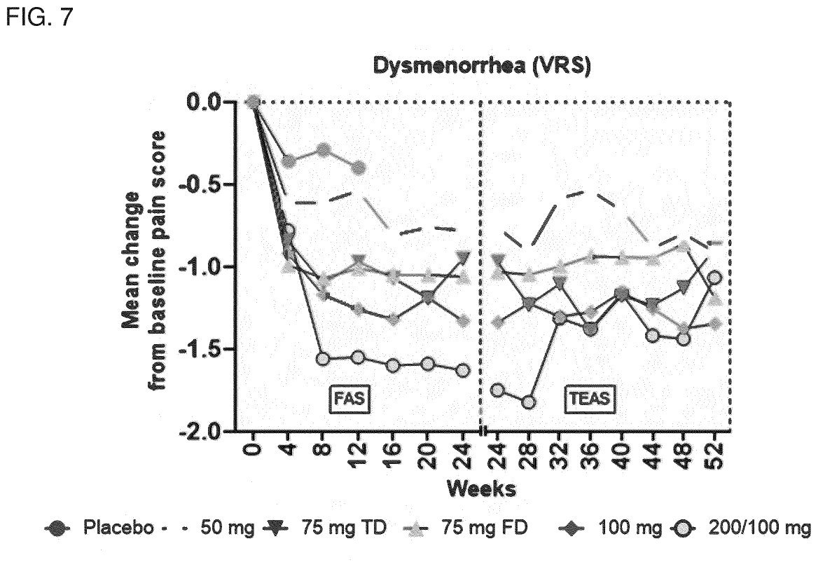

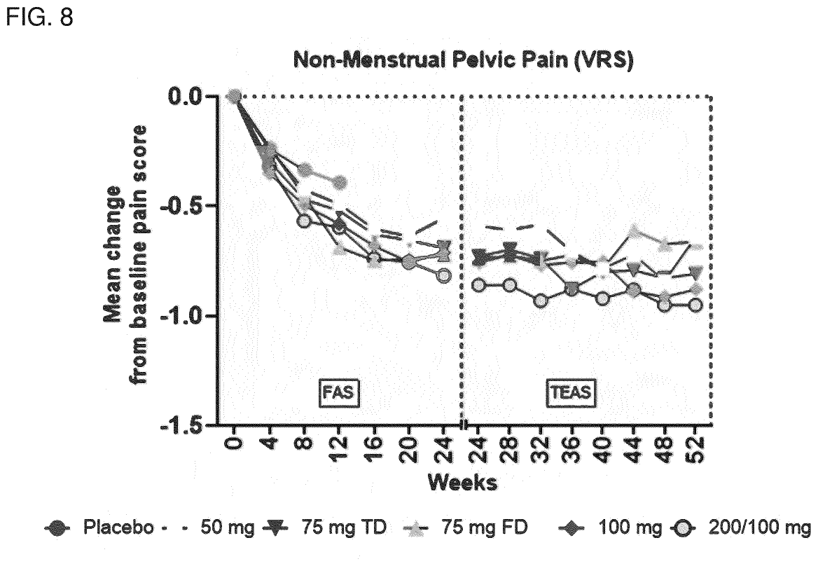

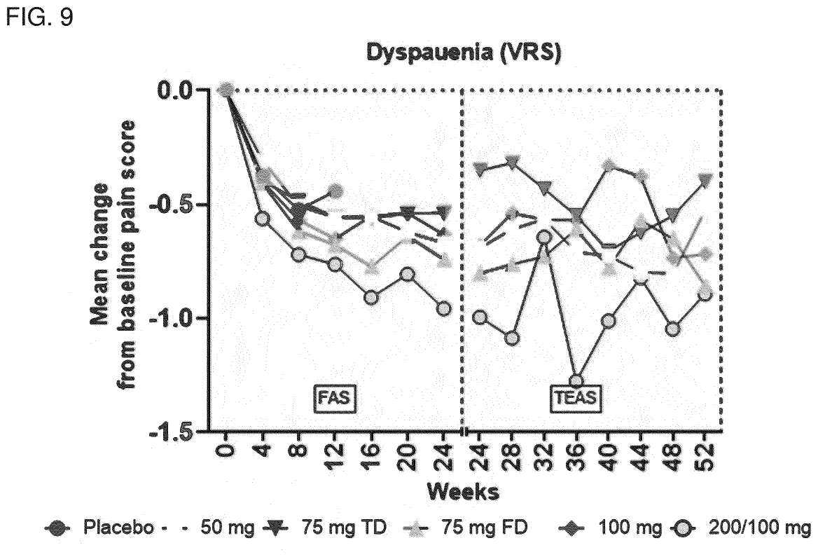

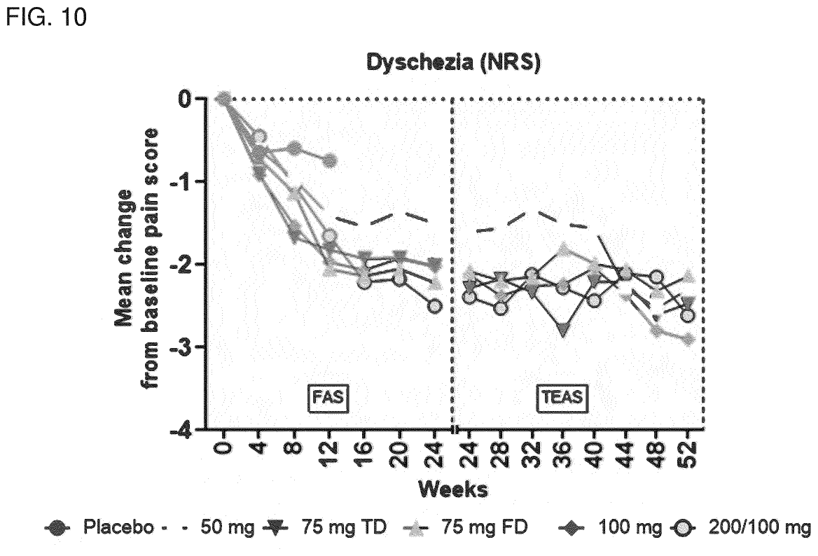

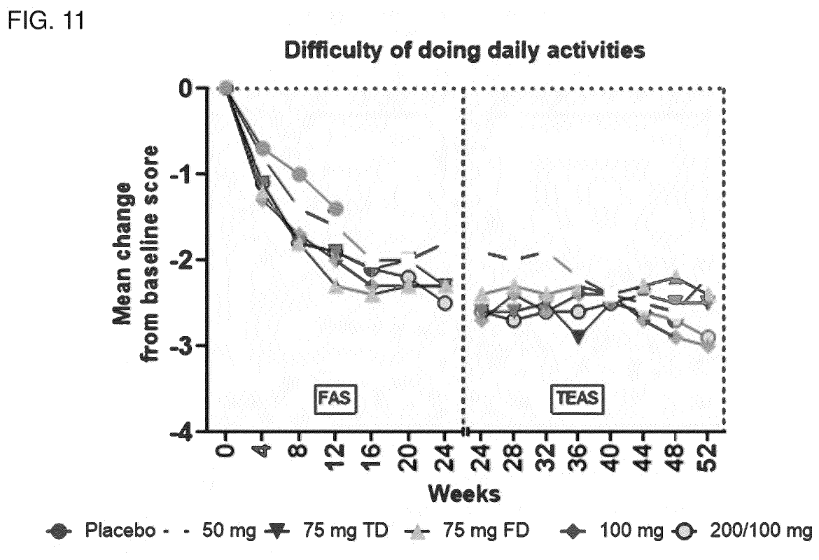

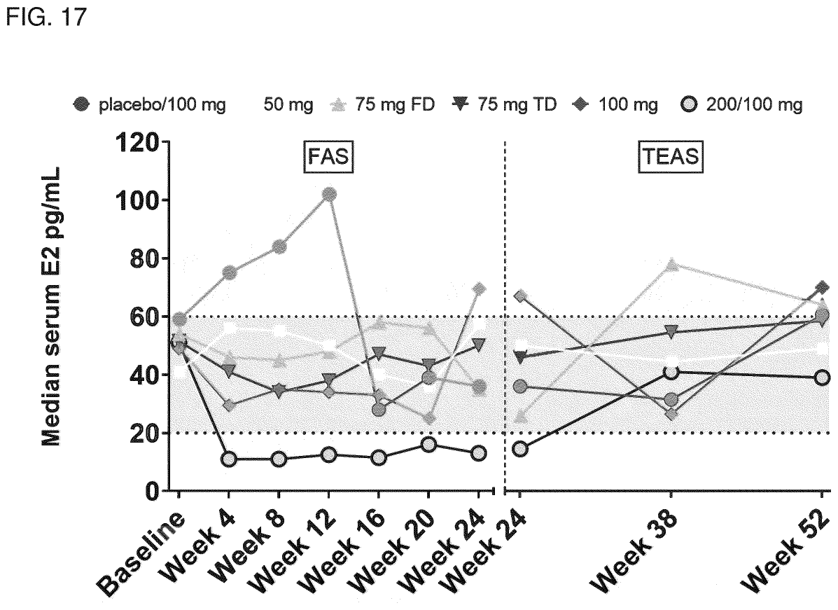

Table 11, below, and FIGS. 3-11 and 17 provide a summary of the effects of 52 weeks of continuous treatment with 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid on patients from all treatment groups. As shown in Table 11, after 12 weeks of treatment, patients exhibited a substantial reduction in symptomology, including OPP, DYS, and NMPP, particularly for doses of 75 mg/day and above compared to placebo. These effects were generally maintained or increased at the 24-weeks and 52-weeks timepoints. Patients in the 200 mg/day treatment group exhibited particularly strong improvements in dyspareunia and dyschezia scores at the 12-week timepoint, which were maintained or increased at 24 and 52 weeks.

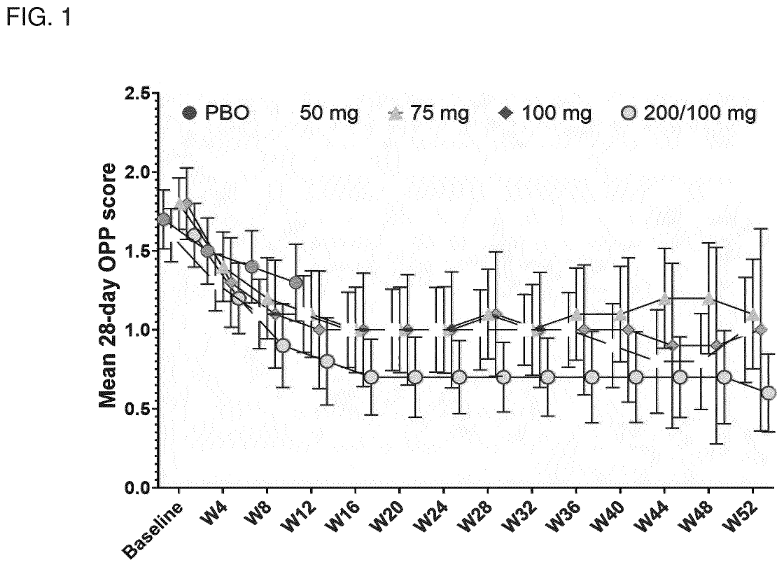

Mean OPP 4-week scores from baseline to week 52 are shown in FIG. 1. As this figure shows, pain scores generally decreased over time, with the highest decreases in the 200 mg/day group and the lowest in placebo. Decreases in pain scores continued after week 12, and were relatively stable after week 24.

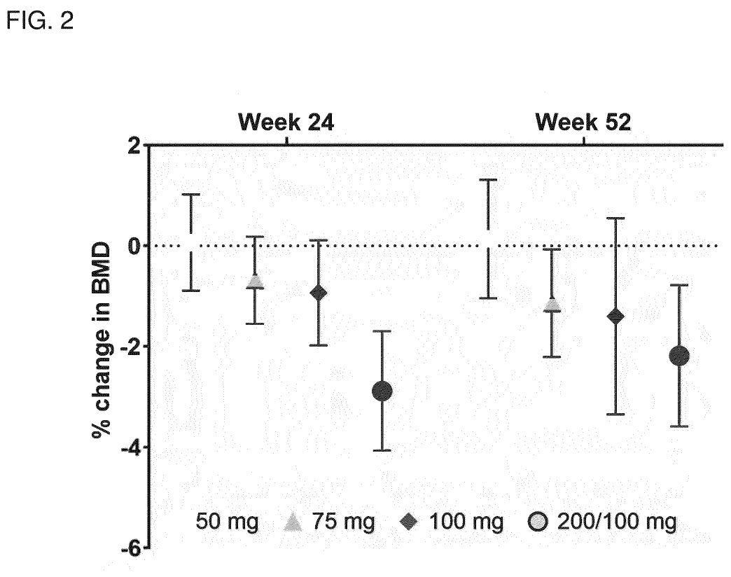

Mean BMD losses, as assessed in the lumbar spine, for each treatment group after 24 weeks and 52 weeks of continuous GnRH antagonist treatment are reported in FIG. 2. As shown in the figure, after 23 weeks of treatment, BMD losses were generally less than 1% at doses of 50 mg/day, 75 mg/day, and 100 mg/day, and were 2.6% for the 200 mg/day treatment group, within the safe limit of 5%. A similar pattern was observed after 52 weeks of continuous treatment. BMD changes in the femur and hip were similar to those observed in the lumbar spine, but were generally smaller.

TABLE 11
Pain assessments after 12 weeks (12 w), 24 weeks (24 w), and 52 weeks (52 w)
of continuous GnRH antagonist treatment

| | | Placebo^{‡} | 50 mg | 75 mg | 100 mg | 200^{†}/100 mg |
|---|---|---|---|---|---|---|
| N | 12 w | 53 | 49 | 56 | 51 | 56 |
| | 24 w | | 40 | 48 | 39 | 44 |
| | 52 w | | 30 | 36 | 22 | 30 |
| % | 12 w | 34.5 | 49.4 | 61.5* | 56.4* | 56.3* |
| responders | 24 w | | 52.5 | 70.8 | 66.7 | 77.3 |
| OPP | 52 w | | 66.7 | 69.2 | 53.8 | 82.4 |
| % | 12 w | 28.5 | 43.3 | 68.2* | 68.6* | 78.9* |
| responders | 24 w | | 47.5 | 58.3 | 82.1 | 84.1 |
| DYS | 52 w | | 50 | 69.2 | 69.2 | 64.7 |
| % | 12 w | 37.1 | 46.2 | 58.5* | 61.5* | 47.7 |
| responders | 24 w | | 50 | 72.9 | 64.1 | 72.7 |
| NMPP | 52 w | | 66.7 | 69.2 | 53.8 | 76.5 |
| Dyspareunia | 12 w | −0.4 (0.9) | −0.6 (0.7) | −0.7 (0.8) | −0.7 (0.9) | −0.8 (1.1)* |
| Mean (SD) | 24 w | | −0.7 (0.8) | −0.7 (0.8) | −0.6 (0.8) | −1.0 (1.0) |
| Change | 52 w | | −0.5 (1.1) | −0.9 (0.8) | −0.7 (1.1) | −0.9 (0.9) |
| from | | | | | | |
| baseline | | | | | | |
| (CFB) | | | | | | |
| Dyschezia | 12 w | −0.7 (1.7) | −1.4 (1.7) | −2.1 (2.5)* | −2.0 (1.8)* | −1.7 (2.3)* |
| Mean (SD) | 24 w | | −1.5 (2.1) | −2.2 (2.5) | −2.0 (2.3) | −2.5 (2.6) |
| CFB | 52 w | | −2.3 (1.8) | −2.1 (2.9) | −2.9 (3.1) | −2.6 (2.7) |

^{‡}PBO only to 12 w;
^{†}Subjects randomized to 200 mg received 100 mg from 24 to 52 w;
*p < 0.05 compared to PBO.

As shown in Tables 9-11, patients that were treated with the GnRH antagonist over the year-long treatment period exhibited a reduction in overall pelvic pain symptoms without incurring a significant loss of bone mineral density, as average bone mineral density losses were less than 5%.

Serum Lipid Levels

Patients treated with the GnRH antagonist exhibited minor percentage increases in LDL-C level, HDL-C level, LDL-C/HDL-C ratio, and serum triglyceride levels, which did not increase further after 24 weeks and 52 weeks of continuous treatment. Except for LDL-C/HDL-C ratio, the largest increases were observed in the 200 mg/day cohort. In this group, after 24 weeks of treatment, patients, on average, exhibited approximately an 8% increase in HDL-C level, a 10% increase in LDL-C level, and a 24% increase in serum triglyceride level relative to baseline. After 52 weeks of treatment, patients exhibited, on average, approximately a 4% increase in HDL-C level, a 10% increase in LDL-C level, and a 32% increase in serum triglyceride level relative to baseline.

Notably, a fasting state was not required for blood sampling except at screening. This may have impacted the triglyceride results, as shown by a 20% increase in the placebo group at Week 12.

In all treatment groups, the percentage of women with LDL-C levels exceeding 160 mg/dL or serum triglyceride levels exceeding 200 mg/dL was less than 10% at most time points.

There were no shifts in LDL-C levels from ≤130 mg/dL at baseline to 190 mg/dL at week 24 or 52. A shift in LDL-C level from ≤160 mg/dL at baseline to 190 mg/dL was reported for one subject in the 100 mg/day cohort at week 24, and for one subject in the placebo/100 mg cohort at week 52. One subject in the 75 mg TD cohort exhibited a shift in serum triglyceride level from ≤300 mg/dL at baseline to >500 mg/dL at Week 24. There were no such increases in serum triglyceride level observed at week 52.

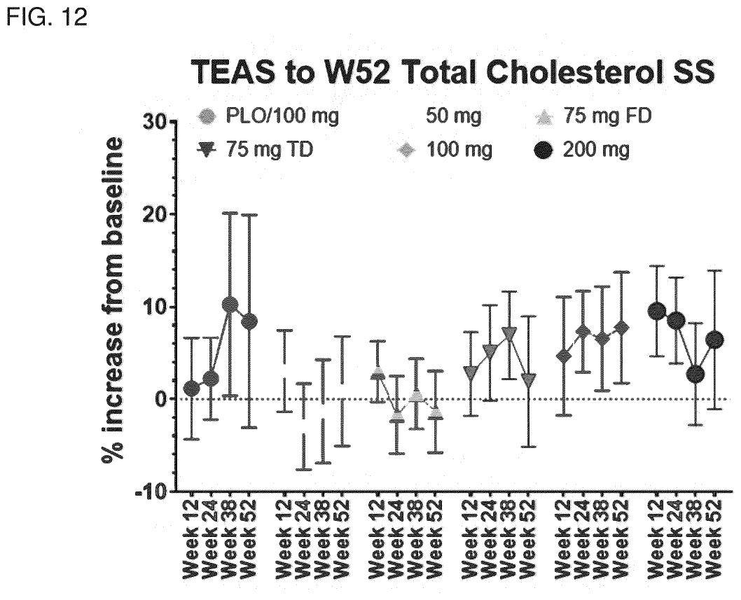

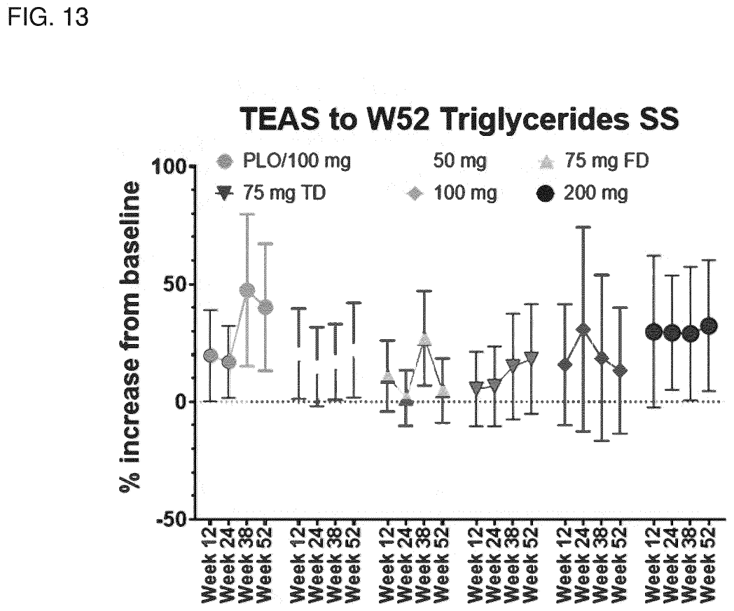

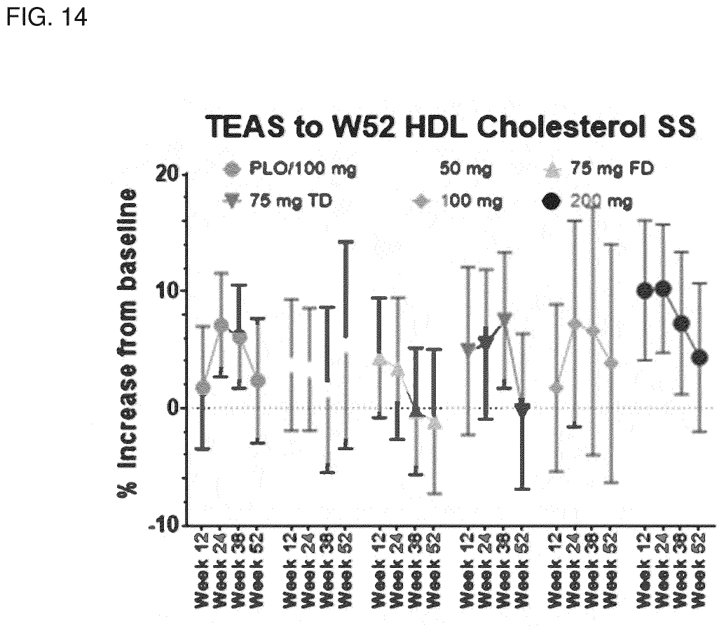

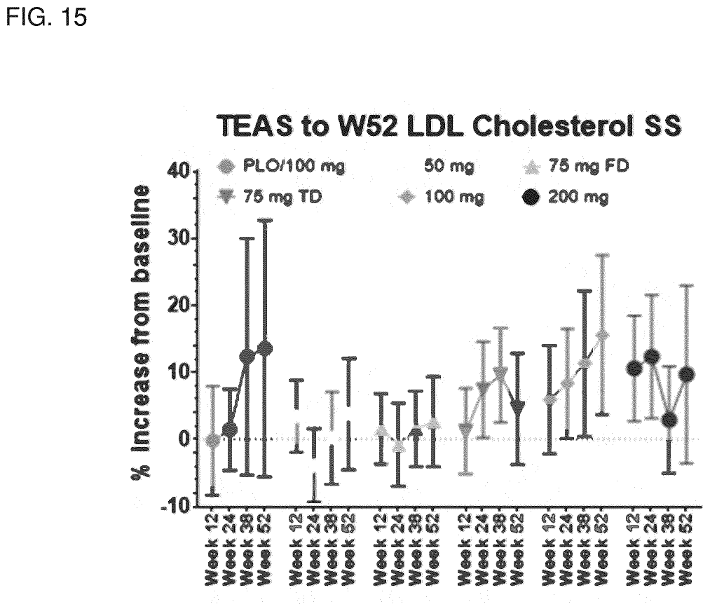

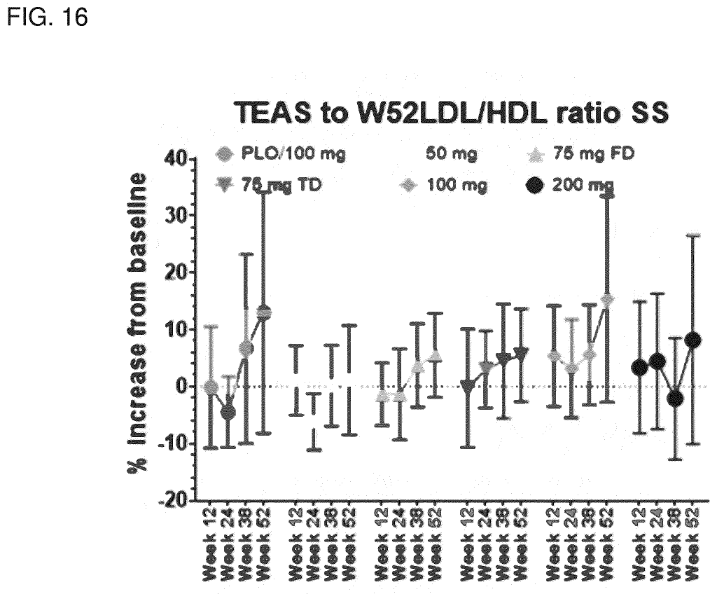

The overall effects of GnRH antagonist treatment on patients' serum lipid levels over the course of this study are summarized in Table 12, below, and in FIGS. 12-16.

TABLE 12
Effects of GnRH antagonist treatment on serum lipid levels

| | | | | | |
|---|---|---|---|---|---|
| Placebo | 50 mg | 75 mg FD | 75 mg TD | 100 mg | 200 mg |
| Cohort | Cohort | Cohort | Cohort | Cohort | Cohort |

Patients with LDL-C level > 160 mg/dl-N (%)

| | | | | | | |
|---|---|---|---|---|---|---|
| Baseline | 3 (5.5) | 0 (0) | 2 (3.4) | 4 (7.1) | 2 (3.8) | 0 (0) |
| Week 24 | 4 (7.3) | 1 (2.0) | 2 (3.4) | 4 (7.1) | 3 (5.8) | 1 (1.8) |

Patients showing an increase in LDL-C level from ≤ 130-160 mg/dl to ≥ 190 mg/dl-N (%)

| | | | | | | |
|---|---|---|---|---|---|---|
| Week 24 | 0 (0) | 0 (0) | 0 (0) | 0 (0) | 1 (1.9) | 0 (0) |

Patients with serum triglyceride level > 200 mg/dl-N (%)

| | | | | | | |
|---|---|---|---|---|---|---|
| Baseline | 4 (7.3) | 2 (4.1) | 2 (3.4) | 6 (10.7) | 5 (9.6) | 4 (7.0) |
| Week 24 | 4 (7.3) | 5 (10.2) | 2 (3.4) | 4 (7.1) | 5 (9.6) | 3 (5.3) |

Patients showing an increase in serum triglyceride level from < 300 mg/dl to > 500 mg/dl-N (%)

| | | | | | | |
|---|---|---|---|---|---|---|
| Week 24 | 0 (0) | 0 (0) | 0 (0) | 1 (1.8) | 0 (0) | 0 (0) |
| TEAS | N = 29 | N = 30 | N = 36 | N = 29 | N = 22 | N = 30 |

Patients with LDL-C level > 160 mg/dl-N (%)

| | | | | | | |
|---|---|---|---|---|---|---|
| Week 52 | 5 (17.2) | 1 (3.3) | 4 (11.1) | 2 (6.9) | 0 (0) | 0 (0) |

Patients showing an increase in LDL-C level from < 160 mg/dl to > 190 mg/dl-N (%)

| | | | | | | |
|---|---|---|---|---|---|---|
| Week 52 | 1 (3.4) | 0 (0) | 0 (0) | 0 (0) | 0 (0) | 0 (0) |

Patients with serum triglyceride level > 200 mg/dl-N (%)

| | | | | | | |
|---|---|---|---|---|---|---|
| Week 52 | 6 (20.7) | 3 (10.0) | 1 (2.8) | 1 (3.4) | 2 (9.1) | 1 (3.3) |

Patients showing an increase in serum triglyceride level from < 300 mg/dl to > 500 mg/dl-N (%)

| | | | | | | |
|---|---|---|---|---|---|---|
| Week 52 | 0 (0) | 0 (0) | 0 (0) | 0 (0) | 0 (0) | 0 (0) |

### CONCLUSIONS

Taken together, the results of these experiments demonstrate that patients administered a GnRH antagonist represented by formula (VI) over the course of an extended, year-long treatment period exhibit a sustained alleviation in symptoms of an estrogen-dependent disease. Patients that were administered the compound at a dose of from 75 mg/day to 200 mg/day exhibited particularly substantial reductions in overall pelvic pain, dysmenorrhea, dyspareunia, dyschezia, and non-menstrual pelvic pain.

Patients that underwent 52 weeks of treatment with compound (VI) generally exhibited a reduction in endometriosis-associated pain after 24 weeks of treatment, and the reduction was maintained with continued treatment through the conclusion of the one-year treatment period. Likewise, quality of life measurements, as assessed by way of patients' PGIC scores, difficulty in performing daily activities, and EHP-30 scores showed maintained improvements after 52 weeks of treatment.

Advantageously, the patients treated with the GnRH antagonist over the lengthy treatment period did not exhibit a significant reduction in bone mineral density, as average bone mineral density losses at the end of the 52-week treatment period remained well within the safe limit of 5%.

### Example 2. Use of a GnRH Antagonist for the Treatment of a Patient Having Uterine Fibroids or Endometriosis

Using the compositions and methods described herein, a patient may be administered a GnRH antagonist so as to treat, and/or ameliorate the symptoms of, uterine fibroids or endometriosis, among other estrogen-dependent diseases. The GnRH antagonist (e.g., a compound of formula (I), above, such as compound (VI) or the choline salt thereof) may be administered to the patient in an amount sufficient to reduce the serum concentration of LH, FSH, and/or E2 in circulation. The GnRH antagonist may be administered, for example, in an amount of from about 25 mg to about 500 mg per dose. Exemplary doses of the GnRH antagonist include, without limitation, an amount of from 25 mg to 500 mg, 30 mg to 495 mg, 35 mg to 490 mg, 40 mg to 485 mg, 45 mg to 480 mg, 50 mg to 475 mg, 55 mg to 470 mg, 60 mg to 465 mg, 65 mg to 460 mg, 70 mg to 455 mg, 75 mg to 450 mg, 80 mg to 445 mg, 85 mg to 440 mg, 90 mg to 435 mg, 95 mg to 430 mg, 100 mg to 425 mg, 105 mg to 420 mg, 110 mg to 415 mg, 115 mg to 410 mg, 120 mg to 405 mg, 125 mg to 400 mg, 130 mg to 395 mg, 135 mg to 390 mg, 140 mg to 385 mg, 145 mg to 380 mg, or 150 mg to 375 mg, per dose. For example, when the GnRH antagonist is compound (VI) or the choline salt thereof, the compound may be administered at a dose of 200 mg.

The GnRH antagonist may be administered to the patient periodically over a treatment period of, for example, 52 or more weeks (e.g., a treatment period lasting 52 weeks, 53 weeks, 54 weeks, 55 weeks, 56 weeks, 57 weeks, 58 weeks, 59 weeks, 60 weeks, 61 weeks, 62 weeks, 63 weeks, 64 weeks, 65 weeks, 66 weeks, 67 weeks, 68 weeks, 69 weeks, 70 weeks, 71 weeks, 72 weeks, 73 weeks, 74 weeks, 75 weeks, 76 weeks, 77 weeks, 78 weeks, 79 weeks, 80 weeks, 81 weeks, 82 weeks, 83 weeks, 84 weeks, 85 weeks, 86 weeks, 87 weeks, 88 weeks, 89 weeks, 90 weeks, 91 weeks, 92 weeks, 93 weeks, 94 weeks, 95 weeks, 96 weeks, 97 weeks, 98 weeks, 99 weeks, 100 weeks, 101 weeks, 102 weeks, 103 weeks, 104 weeks, 105 weeks, 106 weeks, 107 weeks, 108 weeks, 109 weeks, 110 weeks, 111 weeks, 112 weeks, 113 weeks, 114 weeks, 115 weeks, 116 weeks, 117 weeks, 118 weeks, 119 weeks, 120 weeks, 121 weeks, 122 weeks, 123 weeks, 124 weeks, 125 weeks, 126 weeks, 127 weeks, 128 weeks, 129 weeks, 130 weeks, 131 weeks, 132 weeks, 133 weeks, 134 weeks, 135 weeks, 136 weeks, 137 weeks, 138 weeks, 139 weeks, 140 weeks, 141 weeks, 142 weeks, 143 weeks, 144 weeks, 145 weeks, 146 weeks, 147 weeks, 148 weeks, 149 weeks, 150 weeks, or more). The GnRH antagonist may be provided to the patient in combination with add-back therapy. The patient may then be monitored for bone mineral density loss, for example, using dual energy X-ray absorptiometry. The physician may then determine that the patient does not exhibit a substantial reduction in bone mineral density. For example, the physician may determine that, at the conclusion of the treatment period, the patient exhibits a loss of bone mineral density of no greater than 5% (e.g., a reduction in bone mineral density of from 0.1% to 5%, such as a loss of bone mineral density of 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5%).

To determine the responsiveness of the patient to the GnRH antagonist, a physician may monitor the time required for the patient to achieve a reduction in uterine blood loss in the case of a patient having uterine fibroids. For example, the physician may observe one or more of the following responses of the patient as an indicator of successful treatment: (i) a reduction in menstrual blood loss to less than 80 ml per menstrual cycle, such that the patient no longer exhibits heavy menstrual blood loss, the volume of one or more rectovaginal endometriosis nodes following administration of the GnRH antagonist to the patient, and/or (ii) an improvement in the patient's overall well-being as determined by an improvement in the patient's Endometriosis Health Profile questionnaire (EHP-30) score following administration of the GnRH antagonist to the patient and/or by way of a positive Patient Global Impression of Change (PGIC) score following administration of the GnRH antagonist to the patient.

In the case of a patient having endometriosis, the physician may monitor the patient to assess whether the patient exhibits a reduction in pain, such as overall pelvic pain, dysmenorrhea, dyspareunia, or dyschezia. For example, the physician may observe one or more of the following responses of the patient as an indicator of successful treatment: (i) a reduction in serum concentration of FSH, LH, and/or E2 following administration of the GnRH antagonist to the patient, (ii) a reduction in the volume of one or more endometriosis nodes (e.g., rectovaginal endometriosis nodes) following administration of the GnRH antagonist to the patient, (iii) a reduction in bowel involvement of one or more type III endometriosis nodes following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine bleeding following administration of the GnRH antagonist to the patient; (ix) achievement of amenorrhea following administration of the GnRH antagonist to the patient; and/or (x) an improvement in the patient's overall well-being as determined by an improvement in the patient's Endometriosis Health Profile questionnaire (EHP-30) score following administration of the GnRH antagonist to the patient and/or by way of a positive Patient Global Impression of Change (PGIC) score following administration of the GnRH antagonist to the patient.

### Example 3. The GnRH Antagonist Represented by Formula (VI) Effectuates a Sustained Reduction in Uterine Fibroids Symptomology and can Safely be Administered to Patients Over Extended Treatment Periods

### Objective

Uterine fibroids is a common estrogen-dependent disease characterized by the growth of benign tumors of the muscular tissue of the uterus. Uterine fibroids affect women of childbearing age and can vary in size from undetectable to a large bulky mass. The symptoms of uterine fibroids are wide-ranging and include heavy menstrual bleeding, anemia, pelvic pressure and bloating, urinary frequency, and pain that can be extremely debilitating with a significant impact on quality of life. These symptoms can also have an impact on mental health, creating the additional burden of anxiety and distress. The purpose of the experiments described in this example was to evaluate the safety and efficacy of the GnRH antagonist represented by formula (VI), herein, in patients suffering from uterine fibroids.

### Methods

This example describes the results of two Phase 3, double-blind, randomized, placebo-controlled, multicenter, human clinical trials undertaken to evaluate the efficacy of the GnRH antagonist represented by formula (VI) in a series of patients suffering from uterine fibroids. In these trials, referred to herein as “PRIMROSE 1” and “PRIMROSE 2,” human female patients diagnosed as having uterine fibroids were periodically administered compound (VI), 3-[2-fluoro-5-(2,3-difluoro-6-methoxybenzyloxy)4-methoxyphenyl]-2,4-dioxo-1,2,3,4-tetrahydrothieno[3,4d]pyrimidine-5-carboxylic acid, in the form of a choline salt over the course of a 12-month treatment period. Patients were administered compound (VI) once daily in an amount of either 100 mg or 200 mg. Patients receiving compound (VI) in a daily amount of 200 mg also received once daily hormonal add-back therapy (ABT), which consisted of 1.0 mg of β17-estradiol (E2) per day and 0.5 mg of norethindrone acetate (NETA) per day. Patients participating in the PRIMROSE 1 and PRIMROSE 2 studies did not receive Vitamin D or calcium supplementation at any point during the treatment period.

The PRIMROSE 1 study, conducted in the United States, enrolled 526 women. The PRIMROSE 2 study, conducted in the United States and Europe, enrolled 535 women. Baseline characteristics of randomized and treated patients are shown in Table 13, below.

TABLE 13
Demographics and baseline characteristics

| | PRIMROSE 1 | | | PRIMROSE 2 | | |
|---|---|---|---|---|---|---|
| | | | 200 mg + | | | 200 mg + |
| | Placebo | 100 mg | ABT | Placebo | 100 mg | ABT |
| | n = 103 | n = 94 | n = 102 | n = 102 | n = 97 | n = 98 |
| Age (years): | 42.0 (5.7) | 41.3 (5.9) | 41.8 (5.9) | 42.9 (5.3) | 43.4 (5.4) | 43.1 (4.8) |
| mean (SD) | | | | | | |
| White/Black/ | 33/63/4 | | | 95/5/0 | | |
| Other (%) | | | | | | |
| BMI (kg/m²): | 32.2 (6.8) | 33.3 (7.4) | 33.0 (7.3) | 26.8 (5.4) | 27.4 (5.7) | 26.8 (5.5) |
| mean (SD) | | | | | | |
| Weight (kg): | 87.4 (18.9) | 90.4 (22.4) | 88.6 (20.2) | 74.4 (16.0) | 75.2 (15.6) | 72.7 (15.2) |
| mean (SD) | | | | | | |
| Hb < 10 | 26 (25.2) | 31 (33.0) | 31 (30.4) | 14 (13.7) | 21 (21.6) | 24 (24.5) |
| g/dL: n (%) | | | | | | |
| Hb < 12 | 76 (73.8) | 68 (72.3) | 72 (70.6) | 51 (50.0) | 61 (62.9) | 56 (57.1) |
| g/dL: n (%)* | | | | | | |
| MBL** (mL) | 195 (110) | 197 (110) | 195 (117) | 218 (128) | 247 (162) | 213 (143) |
| mean (SD) | | | | | | |

*Patients were characterized as anemic if they exhibited a hemoglobin (Hb) value of less than 12.0 g/dL
**Menstrual blood loss (MBL) was characterized as “heavy” if total blood lost per menstrual cycle exceeded 80 mL, as assessed by way of the alkaline hematin method, described herein.

### Dosing Regimens

Patients were randomized to one of several cohorts: (i) placebo, (ii) 100 mg/day of compound (VI), or (iii) 200 mg/day of compound (VI). Patients receiving 200 mg/day of compound (VI) also received once-daily ABT containing 1.0 mg E2 and 0.5 mg NETA.

### Endpoints

Patients were assessed for uterine fibroid symptomology at baseline, after 24 weeks of treatment, and again after 52 weeks of treatment. Particularly, to monitor the efficacy of compound (VI), patients were evaluated to determine the extent to which they exhibited a reduction in menstrual blood loss relative to their menstrual blood loss patterns at baseline. Patients were also assessed in order to determine the number of days of uterine bleeding during their last 28-day interval prior to week 24 of treatment with placebo or compound (VI).

Apart from analyzing the ability of compound (VI) to engender a reduction in uterine blood loss, patients were also assessed to determine the proportion of subjects that exhibited sustained amenorrhea after 24 weeks of treatment with compound (VI). Patients that exhibited anemia at baseline were evaluated to determine whether they exhibited an improvement in their condition, particularly by assessing whether they exhibited an increase in hemoglobin level during the studies. Pelvic pain is yet another symptom of uterine fibroids; accordingly, patients were also assessed to determine the extent to which they exhibited a reduction in pain during the studies. Pain levels were measured using a Verbal Rating Score (VRS), as described herein. Patients were also assessed to determine the proportion that reported an improvement in quality of life using the Uterine Fibroids Symptoms and Health-Related Quality of Life questionnaire.

To evaluate the safety of compound (VI), patients' bone mineral density levels were measured at baseline and at various timepoints during the studies.

### Results

Patients treated with compound (VI), either in an amount of 100 mg/day without ABT or in an amount of 200 mg/day with once-daily ABT, exhibited a sustained reduction in menstrual blood loss and pain symptoms throughout the duration of the PRIMROSE 1 and PRIMROSE 2 studies. Specifically, in the PRIMROSE 1 study, after 24 weeks of treatment, patients receiving compound (VI) experienced a clinically and statistically significant reduction in menstrual bleeding, defined as 580 mL of menstrual blood loss and a ≥50% reduction in menstrual blood loss from baseline. Among women receiving 200 mg of compound (VI) per day in combination with ABT, 75.5% (p<0.001) achieved a reduction in menstrual blood loss to a level of ≤80 mL and a 50% reduction in menstrual blood loss from baseline. Among patients receiving 100 mg of compound (VI) without ABT, 56.4% (p=0.003) achieved a reduction in menstrual blood loss to a level of ≤80 mL and a ≥50% reduction in menstrual blood loss from baseline. In contrast, among patients receiving placebo, 35% exhibited a reduction in menstrual blood loss to a level of ≤80 mL and a ≥50% reduction in menstrual blood loss from baseline.

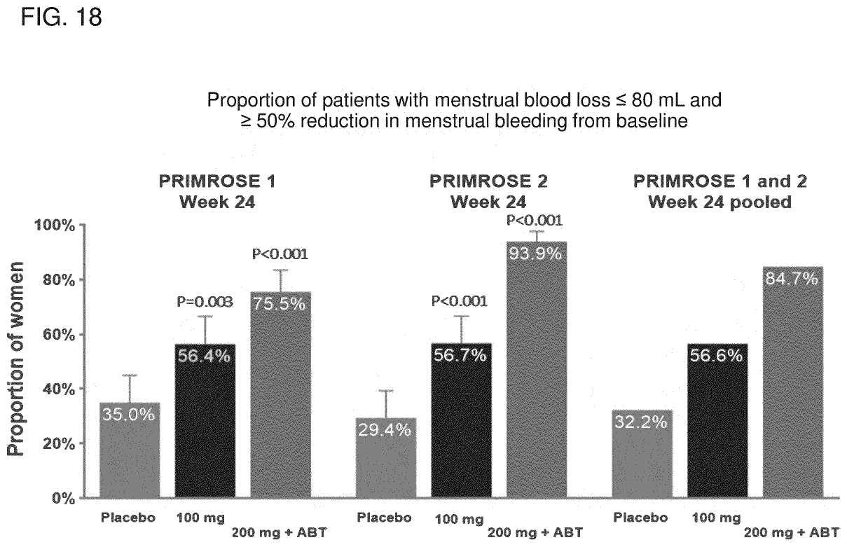

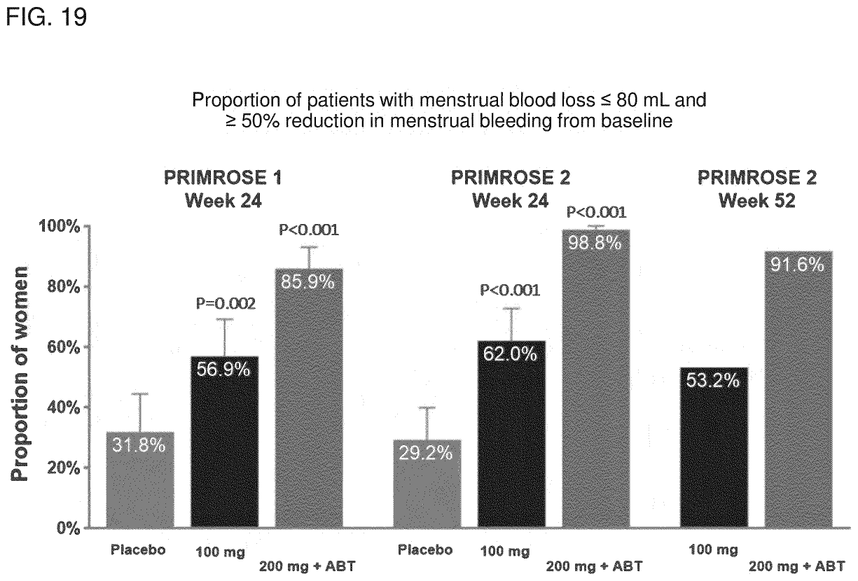

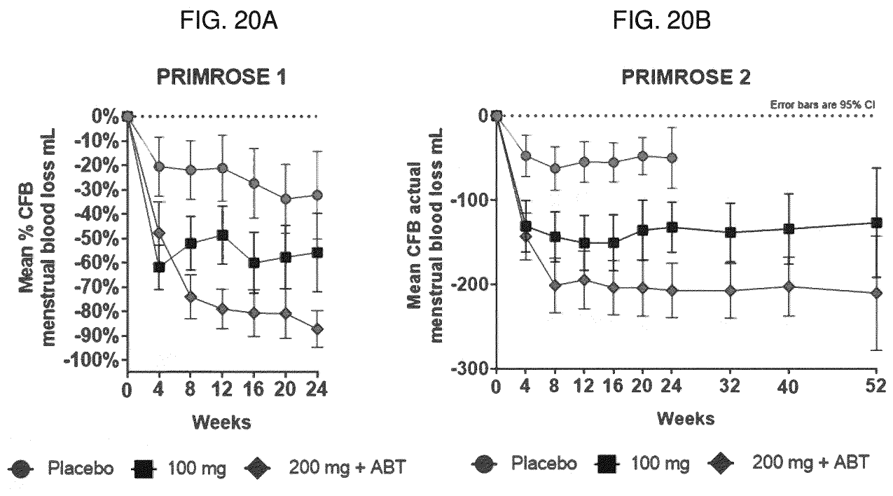

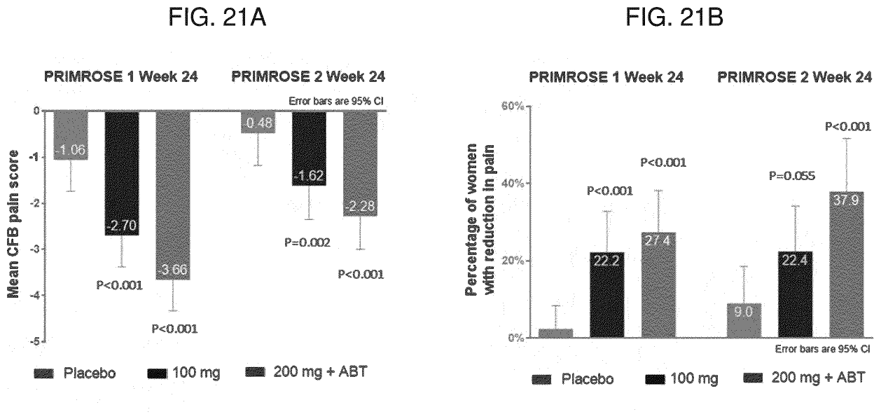

Importantly, the therapeutic effects of compound (VI) were sustained for up to 52 weeks, as demonstrated by the data obtained from the PRIMROSE 2 study. In the PRIMROSE2 trial, among patients being administered 200 mg of compound (VI) per day in combination with ABT for 52 weeks, 91.6% achieved a reduction in menstrual blood loss to a level of ≤80 mL and a ≥50% reduction in menstrual blood loss from baseline. Similarly, among patients receiving 100 mg of compound (VI) without ABT for 52 weeks, 53.2% achieved a reduction in menstrual blood loss to a level of ≤80 mL and a ≥50% reduction in menstrual blood loss from baseline. Both of these values mirror the response rates observed after 24 weeks of treatment in the PRIMROSE 2 study. These results are shown graphically in FIGS. 18-21.

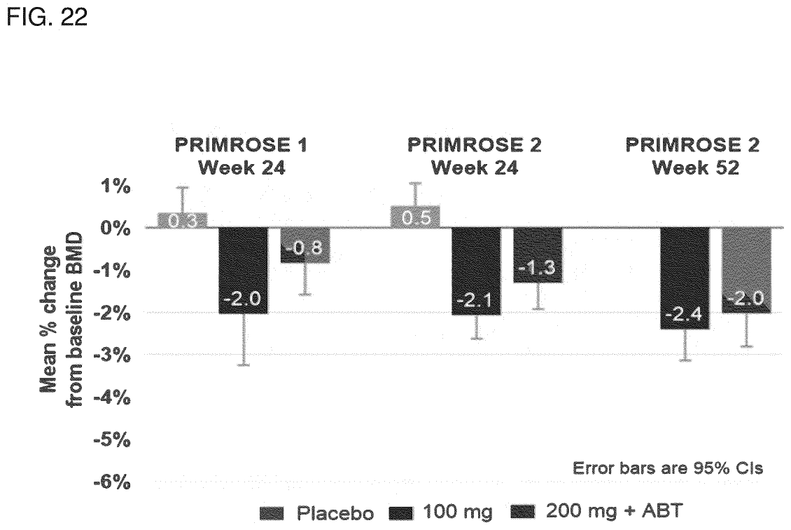

Not only was compound (VI) capable of attenuating uterine blood loss, reducing pain, and improving patients' quality of life, the data from the PRIMROSE 1 and PRIMROSE 2 studies demonstrate that compound (VI) does not induce a substantial decrease in bone mineral density. This is shown, for example, in FIG. 22, which depicts the lumbar spine bone mineral density of patients treated with compound (VI) after 24 weeks and 52 weeks of once-daily treatment with compound (VI).

The efficacy of compound (VI) in the PRIMROSE 1 and PRIMROSE 2 studies is summarized in Table 14, below.

TABLE 14
Efficacy of compound (VI) in reducing uterine
fibroids symptomology and improving quality of
life in PRIMROSE 1 and PRIMROSE 2 studies

| | | | 200 mg/day of | |
|---|---|---|---|---|
| | | | compound (VI) + | |
| | | | once-daily ABT | |
| | 100 mg/day of | | (1.0 mg E2 + 0.5 | |
| | compound (VI) | | mg NETA) | |
| Endpoint | PRIM- | PRIM- | PRIM- | PRIM- |
| | ROSE 1 | ROSE 2 | ROSE 1 | ROSE 2 |
| Reduction in | p = 0.002 | p < 0.001 | p < 0.001 | p < 0.001 |
| menstrual blood | p = 0.001 | p < 0.001 | p < 0.001 | p < 0.001 |
| loss* | | | | |
| Time to | | | | |
| reduced | | | | |
| menstrual | | | | |
| blood loss (≤80 | | | | |
| mL and ≥50% | | | | |
| reduction from | | | | |
| baseline) up to | | | | |
| Week 24 | | | | |
| Number of days | | | | |
| of uterine | | | | |
| bleeding for the | | | | |
| last 28-day | | | | |
| interval prior to | | | | |
| Week 24 | | | | |
| Amenorrhea | p = 0.009 | p < 0.001 | p < 0.001 | p < 0.001 |
| Percentage at | p = 0.007 | p < 0.001 | p < 0.001 | p < 0.001 |
| Week 24 | | | | |
| Time to | | | | |
| amenorrhea up | | | | |
| to Week 24 | | | | |
| Improvement in | p = 0.019 | p = 0.002 | p < 0.001 | p < 0.001 |
| anemia | | | | |
| Hemoglobin level | | | | |
| at week 24 in | | | | |
| anemic subjects⁺ | | | | |
| Reduction in pain | p < 0.001 | p = 0.002 | p < 0.001 | p < 0.001 |
| Change from | | | | |
| baseline pain score | | | | |
| at week 24 | | | | |
| Reduction in | p = 0.149 | p = 0.055 | p = 0.671 | p = 0.008 |
| volume | p = 0.014 | p = 0.003 | p = 0.223 | p < 0.001 |
| Fibroid volume | | | | |
| change from | | | | |
| baseline at | | | | |
| Week 24 | | | | |
| Uterine volume | | | | |
| change from | | | | |
| baseline at | | | | |
| Week 24 | | | | |
| Improvement in | p = 0.002 | p = 0.004 | p < 0.001 | p < 0.001 |
| quality of life | | | | |
| Change from | | | | |
| baseline in quality | | | | |
| of life (QoL) | | | | |
| symptom severity | | | | |
| score (LS mean) | | | | |

⁺Defined as subjects with Hb < 12 g/dL at baseline

### CONCLUSION

As the results of the PRIMROSE 1 and PRIMROSE 2 studies show, compound (VI) can safely be administered to patients over the course of extended treatment periods, such as those having a duration of one year or longer, so as to reduce estrogen-dependent disease symptomology and treat the underlying etiology of the disease. The therapeutic effects of compound (VI) that are observed after initial treatment periods (e.g., of 24 weeks) are observed over extended periods of time (e.g., for 52 weeks or longer). Advantageously, compound (VI) achieves these desirable treatment outcomes without inducing a substantial reduction in bone mineral density.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating an estrogen-dependent disease in a human patient in need thereof, the method comprising periodically administering to the patient a therapeutically effective amount of a gonadotropin-releasing hormone (GnRH) antagonist over the course of a treatment period having a duration of at least 52 weeks.

2. The method of claim 1, wherein the estrogen-dependent disease is uterine fibroids.

3. A method of reducing the volume of menstrual blood loss in a human patient diagnosed as having uterine fibroids, the method comprising periodically administering to the patient a therapeutically effective amount of a GnRH antagonist over the course of a treatment period having a duration of at least 52 weeks.

4. A method of inducing amenorrhea in a human patient diagnosed as having uterine fibroids, the method comprising periodically administering to the patient a therapeutically effective amount of a GnRH antagonist over the course of a treatment period having a duration of at least 52 weeks.

5. A method of reducing the volume of one or more uterine fibroids in a human patient diagnosed as having uterine fibroids, the method comprising periodically administering to the patient a therapeutically effective amount of a GnRH antagonist over the course of a treatment period having a duration of at least 52 weeks.

6. A method of preserving or increasing hemoglobin in a human patient diagnosed as having uterine fibroids, the method comprising periodically administering to the patient a therapeutically effective amount of a GnRH antagonist over the course of a treatment period having a duration of at least 52 weeks.

7. The method of claim 1, wherein the estrogen-dependent disease is endometriosis.

8. A method of reducing the volume of one or more endometriosis nodes in a human patient diagnosed as having endometriosis, the method comprising periodically administering to the patient a therapeutically effective amount of a GnRH antagonist over the course of a treatment period having a duration of at least 52 weeks.

9. A method of reducing pelvic pain in a human patient diagnosed as having endometriosis, the method comprising periodically administering to the patient a therapeutically effective amount of a GnRH antagonist over the course of a treatment period having a duration of at least 52 weeks.

10. A method of reducing dysmenorrhea in a human patient diagnosed as having endometriosis, the method comprising periodically administering to the patient a therapeutically effective amount of a GnRH antagonist over the course of a treatment period having a duration of at least 52 weeks.

11. A method of reducing dyspareunia in a human patient diagnosed as having endometriosis, the method comprising periodically administering to the patient a therapeutically effective amount of a GnRH antagonist over the course of a treatment period having a duration of at least 52 weeks.

12. A method of reducing dyschezia in a human patient diagnosed as having endometriosis, the method comprising periodically administering to the patient a therapeutically effective amount of a GnRH antagonist over the course of a treatment period having a duration of at least 52 weeks.

13. A method of reducing uterine bleeding in a human patient diagnosed as having endometriosis, the method comprising periodically administering to the patient a therapeutically effective amount of a GnRH antagonist over the course of a treatment period having a duration of at least 52 weeks.

14. A method of inducing amenorrhea in a human patient diagnosed as having endometriosis, the method comprising periodically administering to the patient a therapeutically effective amount of a GnRH antagonist over the course of a treatment period having a duration of at least 52 weeks.

15. The method of claim 1, wherein the estrogen-dependent disease is adenomyosis.

16. A method of reducing uterine volume in a human patient diagnosed as having adenomyosis, the method comprising periodically administering to the patient a therapeutically effective amount of a GnRH antagonist over the course of a treatment period having a duration of at least 52 weeks.

17. A method of reducing the thickness of the anterior and/or posterior region of the uterine myometrium in a human patient diagnosed as having adenomyosis, the method comprising periodically administering to the patient a therapeutically effective amount of a GnRH antagonist over the course of a treatment period having a duration of at least 52 weeks.

18. A method of reducing pelvic pain in a human patient diagnosed as having adenomyosis, the method comprising periodically administering to the patient a therapeutically effective amount of a GnRH antagonist over the course of a treatment period having a duration of at least 52 weeks.

19. A method of reducing dysmenorrhea in a human patient diagnosed as having adenomyosis, the method comprising periodically administering to the patient a therapeutically effective amount of a GnRH antagonist over the course of a treatment period having a duration of at least 52 weeks.

20. A method of reducing dyspareunia in a human patient diagnosed as having adenomyosis, the method comprising periodically administering to the patient a therapeutically effective amount of a GnRH antagonist over the course of a treatment period having a duration of at least 52 weeks.

21. A method of reducing dyschezia in a human patient diagnosed as having adenomyosis, the method comprising periodically administering to the patient a therapeutically effective amount of a GnRH antagonist over the course of a treatment period having a duration of at least 52 weeks.

22. A method of reducing uterine tenderness in a human patient diagnosed as having adenomyosis, the method comprising periodically administering to the patient a therapeutically effective amount of a GnRH antagonist over the course of a treatment period having a duration of at least 52 weeks.

23. A method of reducing uterine bleeding in a human patient diagnosed as having adenomyosis, the method comprising periodically administering to the patient a therapeutically effective amount of a GnRH antagonist over the course of a treatment period having a duration of at least 52 weeks.

24. A method of inducing amenorrhea in a human patient diagnosed as having adenomyosis, the method comprising periodically administering to the patient a therapeutically effective amount of a GnRH antagonist over the course of a treatment period having a duration of at least 52 weeks.

25. The method of claim 1, wherein the estrogen-dependent disease is rectovaginal endometriosis.

26. A method of reducing the volume of one or more rectovaginal endometriosis nodes in a human patient diagnosed as having rectovaginal endometriosis, the method comprising periodically administering to the patient a therapeutically effective amount of a GnRH antagonist over the course of a treatment period having a duration of at least 52 weeks.

27. A method of reducing the volume of one or more type Ill rectovaginal endometriosis nodes in a human patient diagnosed as having rectovaginal endometriosis, the method comprising periodically administering to the patient a therapeutically effective amount of a GnRH antagonist over the course of a treatment period having a duration of at least 52 weeks.

28. A method of reducing pelvic pain in a human patient diagnosed as having rectovaginal endometriosis, the method comprising periodically administering to the patient a therapeutically effective amount of a GnRH antagonist over the course of a treatment period having a duration of at least 52 weeks.

29. A method of reducing dysmenorrhea in a human patient diagnosed as having rectovaginal endometriosis, the method comprising periodically administering to the patient a therapeutically effective amount of a GnRH antagonist over the course of a treatment period having a duration of at least 52 weeks.

30. A method of reducing dyspareunia in a human patient diagnosed as having rectovaginal endometriosis, the method comprising periodically administering to the patient a therapeutically effective amount of a GnRH antagonist over the course of a treatment period having a duration of at least 52 weeks.

31. A method of reducing dyschezia in a human patient diagnosed as having rectovaginal endometriosis, the method comprising periodically administering to the patient a therapeutically effective amount of a GnRH antagonist over the course of a treatment period having a duration of at least 52 weeks.

32. A method of reducing uterine bleeding in a human patient diagnosed as having rectovaginal endometriosis, the method comprising periodically administering to the patient a therapeutically effective amount of a GnRH antagonist over the course of a treatment period having a duration of at least 52 weeks.

33. A method of inducing amenorrhea in a human patient diagnosed as having rectovaginal endometriosis, the method comprising periodically administering to the patient a therapeutically effective amount of a GnRH antagonist over the course of a treatment period having a duration of at least 52 weeks.

34. The method of any one of claims 1-33, wherein the GnRH antagonist is a compound represented by formula (I) 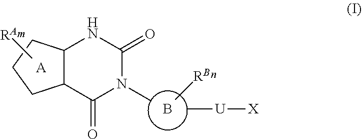
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof.

35. The method of claim 34, wherein the ring A is a thiophene ring represented by formula (IIa)

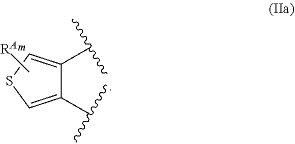

36. The method of claim 34 or 35, wherein m is 1.

37. The method of claim 36, wherein the ring A is an optionally substituted thiophene ring represented by formula (IIb)

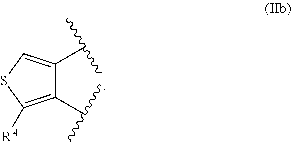

38. The method of any one of claims 34-37, wherein each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

39. The method of claim 38, wherein each R^{A} is COOH or pharmaceutically acceptable salt thereof.

40. The method of any one of claims 34-39, wherein the ring B is an optionally substituted benzene ring, pyridine ring, or thiophene ring.

41. The method of claim 40, wherein the ring B is represented by a formula selected from the group consisting of:

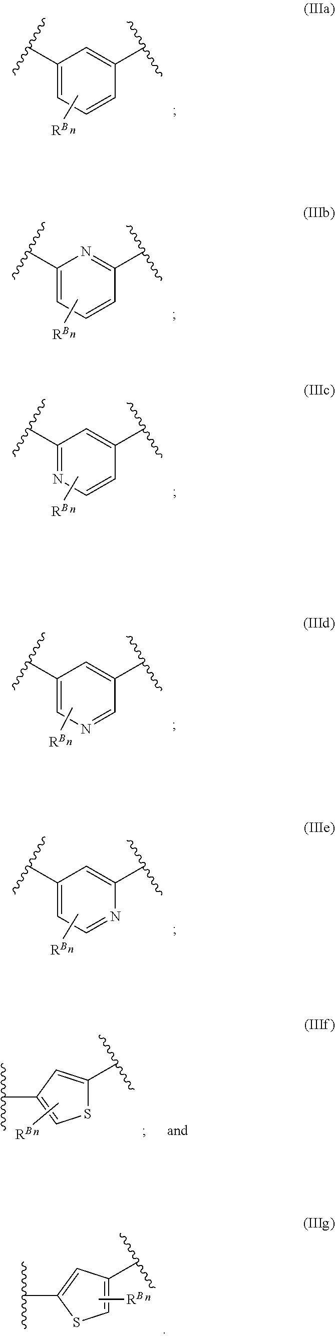

42. The method of any one of claims 34-41, wherein n is 2.

43. The method of claim 42, wherein the ring B is represented by a formula selected from the group consisting of:

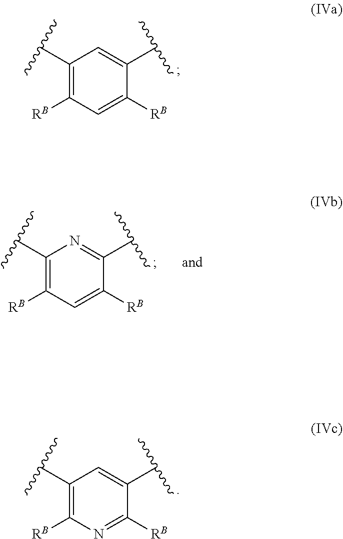

44. The method of any one of claims 34-43, wherein each R^{B} is independently a halogen atom, an optionally substituted lower alkyl group, or OW⁴, wherein each W⁴ is independently a hydrogen atom or an optionally substituted lower alkyl group.

45. The method of claim 44, wherein each R^{B} is independently a fluorine atom, chlorine atom, bromine atom, methyl group, or methoxy group.

46. The method of any one of claims 34-45, wherein U is a single bond.

47. The method of any one of claims 34-46, wherein X is a group represented by —O-L-Y.

48. The method of any one of claims 34-47, wherein L is a methylene group.

49. The method of any one of claims 34-48, wherein Y is an optionally substituted benzene ring represented by formula (V) 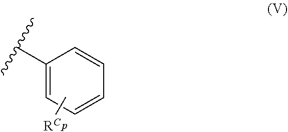
wherein each R^{C} is independently a halogen atom, an optionally substituted lower alkyl group, or OW⁹, wherein each W⁹ is independently a hydrogen atom or an optionally substituted lower alkyl group; and
p is an integer from 0 to 3.

50. The method of claim 49, wherein Y is a substituted benzene ring represented by formula (Va)

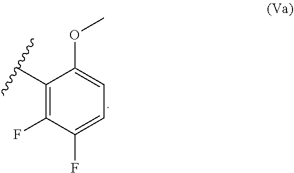 [SMILES: *c1c(OC)ccc(F)c1F]

51. The method of claim 34, wherein the compound is represented by formula (Ia) 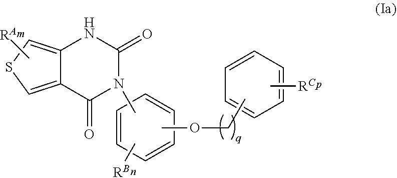
wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
q is an integer from 0 to 3;
each R^{C} is independently a halogen atom, an optionally substituted lower alkyl group, or OW⁹, wherein each W⁹ is independently a hydrogen atom or an optionally substituted lower alkyl group; and
p is an integer from 0 to 3;
or a pharmaceutically acceptable salt thereof.

52. The method of claim 35, wherein the compound is represented by formula (Ib)

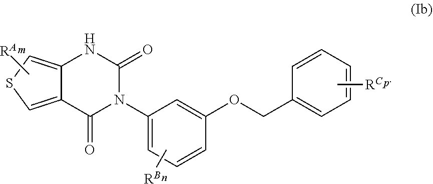

53. The method of claim 36, wherein the compound is represented by formula (Ic) 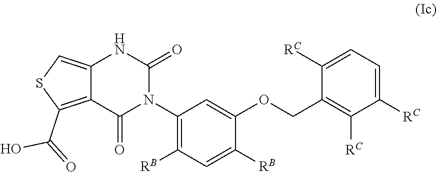
or a pharmaceutically acceptable salt thereof.

54. The method of claim any one of claims 34-53, wherein the compound is represented by formula (VI) 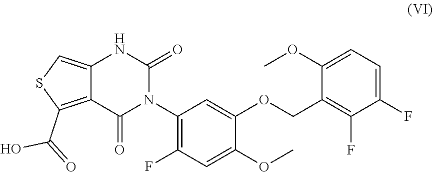 [SMILES: COc1cc(F)c(-n2c(=O)[nH]c3csc(C(=O)O)c3c2=O)cc1OCc1c(OC)ccc(F)c1F]
or a pharmaceutically acceptable salt thereof.

55. The method of claim 54, wherein the compound is the choline salt of the compound represented by formula (VI).

56. The method of claim 55, wherein the compound is in a crystalline state.

57. The method of claim 56, wherein the compound exhibits characteristic X-ray powder diffraction (XRPD) peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ.

58. The method of claim 56 or 57, wherein the compound exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm.

59. The method of any one of claims 56-58, wherein the compound exhibits ¹⁹F solid-state NMR peaks centered at about −151.8 ppm, −145.2 ppm, and −131.6 ppm.

60. The method of any one of claims 34-59, wherein the compound is orally administered to the patient.

61. The method of any one of claims 34-60, wherein the compound is administered to the patient one or more times per day, week, or month during the treatment period.

62. The method of claim 61, wherein the compound is administered to the patient one or more times daily during the treatment period.

63. The method of claim 62, wherein the compound is administered to the patient once daily during the treatment period.

64. The method of any one of claims 61-63, wherein the compound is administered to the patient in an amount of from about 25 mg per day to about 400 mg per day during the treatment period.

65. The method of claim 64, wherein the compound is administered to the patient in an amount of from about 35 mg to about 65 mg per day during the treatment period.

66. The method of claim 65, wherein the compound is administered to the patient in an amount of about 50 mg per day during the treatment period.

67. The method of claim 64, wherein the compound is administered to the patient in an amount of from about 60 mg per day to about 90 mg per day during the treatment period.

68. The method of claim 67, wherein the compound is administered to the patient in an amount of about 75 mg per day during the treatment period.

69. The method of claim 64, wherein the compound is administered to the patient in an amount of from about 85 mg to about 115 mg per day during the treatment period.

70. The method of claim 69, wherein the compound is administered to the patient in an amount of about 100 mg per day during the treatment period.

71. The method of claim 64, wherein the compound is administered to the patient in an amount of from about 185 mg to about 215 mg per day during the treatment period.

72. The method of claim 71, wherein the compound is administered to the patient in an amount of about 200 mg per day during the treatment period.

73. The method of any one of claims 1-33, wherein the GnRH antagonist is elagolix, relugolix, opigolix (ASP1707), BAY-784, or SK-2706.

74. The method of claim 73, wherein the GnRH antagonist is elagolix.

75. The method of claim 74, wherein the GnRH antagonist is orally administered to the patient.

76. The method of claim 74 or 75, wherein the GnRH antagonist is administered to the patient one or more times per day, week, or month during the treatment period.

77. The method of claim 76, wherein the GnRH antagonist is administered to the patient one or more times daily during the treatment period.

78. The method of claim 77, wherein the GnRH antagonist is administered to the patient once daily during the treatment period.

79. The method of any one of claims 74-78, wherein the GnRH antagonist is administered to the patient in an amount of from about 100 mg to about 600 mg per day during the treatment period.

80. The method of claim 79, wherein the GnRH antagonist is administered to the patient in an amount of about 150 mg per day during the treatment period.

81. The method of claim 79, wherein the GnRH antagonist is administered to the patient in an amount of about 300 mg per day during the treatment period.

82. The method of claim 79, wherein the GnRH antagonist is administered to the patient in an amount of about 400 mg per day during the treatment period, optionally wherein the GnRH antagonist is administered to the patient in two daily doses of 200 mg per dose during the treatment period.

83. The method of claim 79, wherein the GnRH antagonist is administered to the patient in an amount of about 600 mg per day during the treatment period, optionally wherein the GnRH antagonist is administered to the patient in two daily doses of 300 mg per dose during the treatment period.

84. The method of claim 73, wherein the GnRH antagonist is relugolix.

85. The method of claim 84, wherein the GnRH antagonist is orally administered to the patient.

86. The method of claim 84 or 85, wherein the GnRH antagonist is administered to the patient one or more times per day, week, or month during the treatment period.

87. The method of claim 86, wherein the GnRH antagonist is administered to the patient one or more times daily during the treatment period.

88. The method of claim 87, wherein the GnRH antagonist is administered to the patient once daily during the treatment period.

89. The method of any one of claims 84-88, wherein the GnRH antagonist is administered to the patient in an amount of from about 10 mg to about 60 mg per day during the treatment period.

90. The method of claim 89, wherein the GnRH antagonist is administered to the patient in an amount of from about 20 mg to about 50 mg per day during the treatment period.

91. The method of claim 90, wherein the GnRH antagonist is administered to the patient in an amount of about 40 mg per day during the treatment period.

92. The method of any one of claims 1-91, wherein the treatment period has a duration of at least 13 months.

93. The method of claim 92, wherein the treatment period has a duration of at least 14 months.

94. The method of claim 93, wherein the treatment period has a duration of at least 15 months.

95. The method of claim 94, wherein the treatment period has a duration of at least 16 months.

96. The method of claim 95, wherein the treatment period has a duration of at least 17 months.

97. The method of claim 96, wherein the treatment period has a duration of at least 18 months.

98. The method of claim 97, wherein the treatment period has a duration of at least 19 months.

99. The method of claim 98, wherein the treatment period has a duration of at least 20 months.

100. The method of claim 99, wherein the treatment period has a duration of at least 21 months.

101. The method of claim 100, wherein the treatment period has a duration of at least 22 months.

102. The method of claim 101, wherein the treatment period has a duration of at least 23 months.

103. The method of claim 102, wherein the treatment period has a duration of at least 24 months.

104. The method of any one of claims 1-91, wherein the treatment period has a duration of from about 12 months to about 60 months.

105. The method of claim 104, wherein the treatment period has a duration of from about 12 months to about 48 months.

106. The method of claim 105, wherein the treatment period has a duration of from about 12 months to about 36 months.

107. The method of claim 106, wherein the treatment period has a duration of from about 12 months to about 24 months.

108. The method of any one of claims 1-91, wherein the treatment period has a duration of about 12 months.

109. The method of any one of claims 1-91, wherein the treatment period has a duration of about 18 months.

110. The method of any one of claims 1-91, wherein the treatment period has a duration of about 24 months.

111. The method of any one of claims 1-91, wherein the treatment period has a duration of about 30 months.

112. The method of any one of claims 1-91, wherein the treatment period has a duration of about 36 months.

113. The method of any one of claims 1-91, wherein the treatment period has a duration of about 42 months.

114. The method of any one of claims 1-91, wherein the treatment period has a duration of about 48 months.

115. The method of any one of claims 1-91, wherein the treatment period has a duration of about 54 months.

116. The method of any one of claims 1-91, wherein the treatment period has a duration of about 60 months.

117. The method of any one of claims 1-116, wherein add-back therapy is periodically administered to the patient during the treatment period.

118. The method of claim 117, wherein the add-back therapy is administered to the patient one or more times daily during the treatment period.

119. The method of claim 118, wherein the add-back therapy is administered to the patient once daily, concurrently with the GnRH antagonist, during the treatment period.

120. The method of claim 118, wherein the add-back therapy is administered to the patient once daily, prior to administration of the GnRH antagonist, during the treatment period.

121. The method of claim 118, wherein the add-back therapy is administered to the patient once daily, following administration of the GnRH antagonist, during the treatment period.

122. The method of claim 121, wherein the add-back therapy is administered to the patient in the form of a pharmaceutical composition comprising the GnRH antagonist.

123. The method of any one of claims 117-122, wherein the add-back therapy comprises an estrogen.

124. The method of claim 123, wherein the estrogen is selected from the group consisting of β17-estradiol, ethinyl estradiol, and conjugated estrogens.

125. The method of claim 124, wherein the estrogen is β17-estradiol.

126. The method of claim 125, wherein the β17-estradiol is administered to the patient in an amount of about 1.0 mg/day during the treatment period.

127. The method of claim 125, wherein the β17-estradiol is administered to the patient in an amount of about 0.5 mg/day during the treatment period.

128. The method of claim 124, wherein the estrogen is ethinyl estradiol.

129. The method of claim 128, wherein the ethinyl estradiol is administered to the patient in an amount of about 5.0 μg/day during the treatment period.

130. The method of claim 128, wherein the ethinyl estradiol is administered to the patient in an amount of about 2.5 μg/day during the treatment period.

131. The method of claim 124, wherein the estrogen is a conjugated estrogen.

132. The method of claim 131, wherein the conjugated estrogen is administered to the patient in an amount of about 0.625 mg/day during the treatment period.

133. The method of claim 131, wherein the conjugated estrogen is administered to the patient in an amount of about 0.45 mg/day during the treatment period.

134. The method of claim 131, wherein the conjugated estrogen is administered to the patient in an amount of about 0.3 mg/day during the treatment period.

135. The method of any one of claims 117-134, wherein the add-back therapy comprises a progestin.

136. The method of claim 135, wherein the progestin is selected from the group consisting of norethindrone or an ester thereof, progesterone, norgestimate, medroxyprogesterone, and drospirenone.

137. The method of claim 136, wherein the progestin is norethindrone or norethindrone acetate.

138. The method of claim 137, wherein the norethindrone or norethindrone acetate is administered to the patient in an amount of about 1.0 mg/day during the treatment period.

139. The method of claim 137, wherein the norethindrone or norethindrone acetate is administered to the patient in an amount of about 0.5 mg/day during the treatment period.

140. The method of claim 137, wherein the norethindrone or norethindrone acetate is administered to the patient in an amount of about 0.1 mg/day during the treatment period.

141. The method of claim 136, wherein the progestin is progesterone.

142. The method of claim 141, wherein the progesterone is administered to the patient in an amount of about 200 mg/day during the treatment period.

143. The method of claim 141, wherein the progesterone is administered to the patient in an amount of about 100 mg/day during the treatment period.

144. The method of claim 136, wherein the progestin is norgestimate.

145. The method of claim 144, wherein the norgestimate is administered to the patient in an amount of about 0.09 mg/day during the treatment period.

146. The method of claim 136, wherein the progestin is medroxyprogesterone.

147. The method of claim 146, wherein the medroxyprogesterone is administered to the patient in an amount of about 5 mg/day during the treatment period.

148. The method of claim 146, wherein the medroxyprogesterone is administered to the patient in an amount of about 2.5 mg/day during the treatment period.

149. The method of claim 146, wherein the medroxyprogesterone is administered to the patient in an amount of about 1.5 mg/day during the treatment period.

150. The method of claim 136, wherein the progestin is drospirenone.

151. The method of claim 150, wherein the drospirenone is administered to the patient in an amount of about 0.5 mg/day during the treatment period.

152. The method of claim 150, wherein the drospirenone is administered to the patient in an amount of about 0.25 mg/day during the treatment period.

153. The method of any one of claims 117-152, wherein the add-back therapy comprises about 1.0 mg of β17-estradiol and about 0.5 mg of norethindrone acetate.

154. The method of any one of claims 117-152, wherein the add-back therapy comprises about 0.5 mg of β17-estradiol and about 0.1 mg of norethindrone acetate.

155. The method of any one of claims 1-154, wherein the patient is a pre-menopausal female of from about 18 to about 48 years of age.

156. The method of any one of claims 1-155, wherein the patient has been determined to exhibit a serum concentration of follicle-stimulating hormone (FSH) of about 20 IU/L or less prior to administration of the GnRH antagonist to the patient.

157. The method of any one of claims 1-156, wherein the patient has been determined to exhibit a rectal (type II) and/or vaginal (type Ill) endometriosis node of at least 2 cm prior to administration of the GnRH antagonist to the patient.

158. The method of claim 157, wherein the length of the type II and/or type Ill endometriosis node is assessed by way of magnetic resonance imaging (MRI).

159. The method of any one of claims 1-158, wherein the patient has been determined to exhibit a junctional-zone width of about 12 mm or more prior to administration of the GnRH antagonist to the patient.

160. The method of claim 159, wherein the junctional-zone width is assessed by way of MRI.

161. The method of any one of claims 1-160, wherein the patient exhibits a reduction in serum concentration of luteinizing hormone (LH), follicle-stimulating hormone (FSH), and/or β17-estradiol (E2) following administration of the GnRH antagonist to the patient.

162. The method of claim 161, wherein the patient exhibits the reduction in serum concentration of LH, FSH, and/or E2 within from about one day to about 36 weeks of the onset of administration of the GnRH antagonist to the patient.

163. The method of any one of claims 1, 2, 5-12, 15-22, 25-31, and 34-162, wherein the patient exhibits a reduction in uterine bleeding following administration of the GnRH antagonist to the patient.

164. The method of any one of claims and 3, 13, 23, 32, and 163, wherein the patient exhibits the reduction in uterine bleeding within from about one day to about 36 weeks of the onset of administration of the GnRH antagonist to the patient.

165. The method of any one of claims 3, 13, 23, 32, 163, and 164, wherein the reduction in uterine bleeding is assessed by way of an alkaline hematin method.

166. The method of any one of claims 1-3, 5-13, 15-23, 25-32, and 34-165, wherein the patient exhibits amenorrhea following administration of the GnRH antagonist to the patient.

167. The method of any one of claims 4, 14, 24, 33, and 166, wherein the patient exhibits the amenorrhea within from about one day to about 36 weeks of the onset of administration of the GnRH antagonist to the patient.

168. The method of any one of claims 1-7, 9-25, and 28-167, wherein the patient exhibits a reduction in the volume of one or more rectovaginal endometriosis nodes following administration of the GnRH antagonist to the patient.

169. The method of any one of claims 8, 26, 27, and 168, wherein the patient exhibits the reduction in volume of the one or more rectovaginal endometriosis nodes within from about one day to about 36 weeks of the onset of administration of the GnRH antagonist to the patient.

170. The method of any one of claims 8, 26, 27, 168, and 169, wherein the reduction in volume of the one or more rectovaginal endometriosis nodes is assessed by way of MRI or TVUS.

171. The method of any one of claims 1-170, wherein the patient exhibits a reduction in bowel involvement of one or more type Ill endometriosis nodes following administration of the GnRH antagonist to the patient.

172. The method of claim 171, wherein the patient exhibits the reduction in bowel involvement of the one or more type Ill endometriosis nodes within from about one day to about 36 weeks of the onset of administration of the GnRH antagonist to the patient.

173. The method of any one of claims 1-8, 10-17, 19-27, and 29-172, wherein the patient exhibits a reduction in pelvic pain following administration of the GnRH antagonist to the patient.

174. The method of any one of claims 9, 18, 28, and 173, wherein the patient exhibits the reduction in pelvic pain within from about one day to about 36 weeks of the onset of administration of the GnRH antagonist to the patient.

175. The method of any one of claims 9, 18, 28, 173, and 174, wherein the reduction in pelvic pain is assessed by way of a modified Biberoglu & Behrman (mB&B) score, Numerical Rating Scale (NRS) score, or Verbal Rating Scale (VRS) score.

176. The method of any one of claims 1-9, 11-18, 20-28, and 39-175, wherein the patient exhibits a reduction in dysmenorrhea following administration of the GnRH antagonist to the patient.

177. The method of any one of claims 10, 19, 29, and 176, wherein the patient exhibits the reduction in dysmenorrhea within from about one day to about 36 weeks of the onset of administration of the GnRH antagonist to the patient.

178. The method of any one of claims 10, 19, 29, 176, and 177, wherein the reduction in dysmenorrhea is assessed by way of an mB&B score, NRS score, or VRS score.

179. The method of any one of claims 1-10, 12-19, 21-29, and 31-178, wherein the patient exhibits a reduction in dyspareunia following administration of the GnRH antagonist to the patient.

180. The method of any one of claims 11, 20, 30, and 179, wherein the patient exhibits the reduction in dyspareunia within from about one day to about 36 weeks commencement of the onset of administration of the GnRH antagonist to the patient.

181. The method of any one of claims 11, 20, 30, 179, and 180, wherein the reduction in dyspareunia is assessed by way of an mB&B score, NRS score, or VRS score.

182. The method of any one of claims 1-11, 13-20, 22-30, and 32-181, wherein the patient exhibits a reduction in dyschezia following administration of the GnRH antagonist to the patient.

183. The method of any one of claims 12, 21, 31, and 182, wherein the patient exhibits the reduction in dyschezia within from about one day to about 36 weeks of the onset of administration of the GnRH antagonist to the patient.

184. The method of any one of claims 12, 21, 31, 182, and 183, wherein the reduction in dyschezia is assessed by way of an mB&B score, NRS score, or VRS score.

185. The method of any one of claims 1-15 and 17-184, wherein the patient exhibits a reduction in uterine volume following administration of the GnRH antagonist to the patient.

186. The method of any one of claims 16 and 185, wherein the patient exhibits the reduction in uterine volume within from about one day to about 36 weeks of the onset of administration of the GnRH antagonist to the patient.

187. The method of any one of claims 16, 185, and 186, wherein the reduction in uterine volume is assessed by way of MRI or transvaginal ultrasound (TVUS).

188. The method of any one of claims 1-16 and 18-187, wherein the patient exhibits a reduction in the thickness of the anterior and/or posterior region of the uterine myometrium following administration of the GnRH antagonist to the patient.

189. The method of any one of claims 17 and 188, wherein the patient exhibits the reduction in thickness of the anterior and/or posterior region of the uterine myometrium within from about one day to about 36 weeks of the onset of administration of the GnRH antagonist to the patient.

190. The method of any one of claims 1-21 and 23-189, wherein the patient exhibits a reduction in uterine tenderness following administration of the GnRH antagonist to the patient.

191. The method of any one of claims 22 and 190, wherein the patient exhibits the reduction in uterine tenderness within from about one day to about 36 weeks of the onset of administration of the GnRH antagonist to the patient.

192. The method of any one of claims 1-191, wherein the patient exhibits a reduction in the diameter of a junctional zone of adenomyosis following administration of the GnRH antagonist to the patient.

193. The method of claim 192, wherein the patient exhibits the reduction in the diameter of the junctional zone of adenomyosis within from about one day to about 36 weeks of the onset of administration of the GnRH antagonist to the patient.

194. The method of any one of claims 1-193, wherein the patient exhibits an improvement in Endometriosis Health Profile questionnaire (EHP-30) score following administration of the GnRH antagonist to the patient.

195. The method of claim 194, wherein the patient exhibits the improvement in the EHP-30 score within from about one day to about 36 weeks of the onset of administration of the GnRH antagonist to the patient.

196. The method of any one of claims 1-195, wherein the patient exhibits a positive Patient Global Impression of Change (PGIC) score core following administration of the GnRH antagonist to the patient.

197. The method of claim 196, wherein the patient exhibits the positive PGIC score within from about one day to about 36 weeks of the onset of administration of the GnRH antagonist to the patient.

198. The method of any one of claims 1-197, the method further comprising monitoring the patient's bone mineral density (BMD) at the end of the treatment period.

199. The method of claim 198, the method further comprising determining that the patient does not exhibit a reduction in BMD of greater than 5% at the end of the treatment period relative to a measurement of the patient's BMD obtained prior to, or during, the treatment period.

200. The method of any one of claims 1-199, wherein the patient does not exhibit a reduction in BMD of greater than 5% at the end of the treatment period relative to a measurement of the patient's BMD obtained prior to, or during, the treatment period.

201. The method of claim 200, wherein the patient does not exhibit a reduction in BMD of greater than 4% at the end of the treatment period relative to a measurement of the patient's BMD obtained prior to, or during, the treatment period.

202. The method of claim 201, wherein the patient does not exhibit a reduction in BMD of greater than 3% at the end of the treatment period relative to a measurement of the patient's BMD obtained prior to, or during, the treatment period.

203. The method of claim 202, wherein the patient does not exhibit a reduction in BMD of greater than 2% at the end of the treatment period relative to a measurement of the patient's BMD obtained prior to, or during, the treatment period.

204. The method of claim 203, wherein the patient does not exhibit a reduction in BMD of greater than 1% at the end of the treatment period relative to a measurement of the patient's BMD obtained prior to, or during, the treatment period.

205. The method of any one of claims 198-204, wherein the BMD is assessed by dual energy X-ray absorptiometry.

206. The method of claim 205, wherein the BMD is assessed in the spine or femur of the patient.

207. The method of any one of claims 198-204, wherein the BMD is assessed by comparing the concentration of bone specific alkaline phosphatase (BAP) in a sample isolated from the patient following the administration of the GnRH antagonist to the concentration of BAP in a sample isolated from the patient prior to the administration of the GnRH antagonist.

208. The method of any one of claims 198-204, wherein the BMD is assessed by comparing the concentration of deoxypyridinoline (DPD) in a sample isolated from the patient following the administration of the GnRH antagonist to the concentration of DPD in a sample isolated from the patient prior to the administration of the GnRH antagonist.

209. The method of any one of claims 198-204, wherein the BMD is assessed by comparing the concentration of type I collagen C-terminal telopeptide (CTX) in a sample isolated from the patient following the administration of the GnRH antagonist to the concentration of CTX in a sample isolated from the patient prior to the administration of the GnRH antagonist.

210. The method of any one of claims 198-204, wherein the BMD is assessed by comparing the concentration of procollagen 1 N-terminal peptide (P1NP) in a sample isolated from the patient following the administration of the GnRH antagonist to the concentration of P1NP in a sample isolated from the patient prior to the administration of the GnRH antagonist.

211. The method of any one of claims 1-210, wherein the patient does not exhibit an increase in total cholesterol level of greater than 10% after from about 6 weeks to about 52 weeks of being administered the GnRH antagonist during the treatment period relative to a measurement of the patient's total cholesterol level obtained prior to the treatment period.

212. The method of any one of claims 1-211, wherein the patient does not exhibit an increase in low-density lipoprotein-cholesterol level of greater than 10% after from about 6 weeks to about 52 weeks of being administered the GnRH antagonist during the treatment period relative to a measurement of the patient's low-density lipoprotein-cholesterol level obtained prior to the treatment period.

213. The method of any one of claims 1-212, wherein the patient does not exhibit an increase in their ratio of low-density lipoprotein-cholesterol to high-density lipoprotein-cholesterol of greater than 10% after from about 6 weeks to about 52 weeks of being administered the GnRH antagonist during the treatment period relative to a measurement of the patient's ratio of low-density lipoprotein-cholesterol to high-density lipoprotein-cholesterol obtained prior to the treatment period.

214. The method of any one of claims 1-213, wherein the patient does not exhibit an increase in low-density lipoprotein-cholesterol level to a level greater than 160 mg/dl after from about 6 weeks to about 52 weeks of being administered the GnRH antagonist during the treatment period.

215. The method of any one of claims 1-214, wherein the patient does not exhibit an increase in low-density lipoprotein-cholesterol level from a level less than 160 mg/dl (e.g., from a level less than 130 mg/dl) to a level greater than 190 mg/dl after from about 6 weeks to about 52 weeks of being administered the GnRH antagonist during the treatment period.

216. The method of any one of claims 1-215, wherein the patient does not exhibit an increase in serum triglyceride level of greater than 30% after from about 6 weeks to about 52 weeks of being administered the GnRH antagonist during the treatment period relative to a measurement of the patient's serum triglyceride level obtained prior to the treatment period.

217. The method of any one of claims 1-216, wherein the patient does not exhibit an increase in serum triglyceride level to a level greater than 200 mg/dl after from about 6 weeks to about 52 weeks of being administered the GnRH antagonist during the treatment period.

218. The method of any one of claims 1-217, wherein the patient does not exhibit an increase in serum triglyceride level from a level less than 300 mg/dl to a level greater than 500 mg/dl after from about 6 weeks to about 52 weeks of being administered the GnRH antagonist during the treatment period.

219. The method of any one of claims 1-218, the method comprising:
a) monitoring the patient's total cholesterol level after from about 6 weeks to about 52 weeks of being administered the GnRH antagonist during the treatment period;
b) determining that the patient does not exhibit an increase in total cholesterol level of greater than 10% relative to a measurement of the patient's total cholesterol level obtained prior to the treatment period; and
c) continuing to administer the GnRH antagonist to the patient for the remainder of the treatment period.

220. The method of any one of claims 1-219, the method comprising:
a) monitoring the patient's low-density lipoprotein-cholesterol level after from about 6 weeks to about 52 weeks of being administered the GnRH antagonist during the treatment period;
b) determining that the patient does not exhibit an increase in low-density lipoprotein-cholesterol level of greater than 10% relative to a measurement of the patient's low-density lipoprotein-cholesterol level obtained prior to the treatment period; and
c) continuing to administer the GnRH antagonist to the patient for the remainder of the treatment period.

221. The method of any one of claims 1-220, the method comprising:
a) monitoring the patient's ratio of low-density lipoprotein-cholesterol to high-density lipoprotein-cholesterol after from about 6 weeks to about 52 weeks of being administered the GnRH antagonist during the treatment period;
b) determining that the patient does not exhibit an increase in their ratio of low-density lipoprotein-cholesterol to high-density lipoprotein-cholesterol of greater than 10% relative to a measurement of the patient's ratio of low-density lipoprotein-cholesterol to high-density lipoprotein-cholesterol obtained prior to the treatment period; and
c) continuing to administer the GnRH antagonist to the patient for the remainder of the treatment period.

222. The method of any one of claims 1-221, the method comprising:
a) monitoring the patient's low-density lipoprotein-cholesterol level after from about 6 weeks to about 52 weeks of being administered the GnRH antagonist during the treatment period;
b) determining that the patient does not exhibit an increase in low-density lipoprotein-cholesterol level to a level greater than 160 mg/dl; and
c) continuing to administer the GnRH antagonist to the patient for the remainder of the treatment period.

223. The method of any one of claims 1-222, the method comprising:
a) monitoring the patient's low-density lipoprotein-cholesterol level after from about 6 weeks to about 52 weeks of being administered the GnRH antagonist during the treatment period;
b) determining that the patient does not exhibit an increase in low-density lipoprotein-cholesterol level from a level less than 160 mg/dl (e.g., from a level less than 130 mg/dl) to a level greater than 190 mg/dl; and
c) continuing to administer the GnRH antagonist to the patient for the remainder of the treatment period.

224. The method of any one of claims 1-223, the method comprising:
a) monitoring the patient's serum triglyceride level after from about 6 weeks to about 52 weeks of being administered the GnRH antagonist during the treatment period;
b) determining that the patient does not exhibit an increase in serum triglyceride level of greater than 30% relative to a measurement of the patient's serum triglyceride level obtained prior to the treatment period; and
c) continuing to administer the GnRH antagonist to the patient for the remainder of the treatment period.

225. The method of any one of claims 1-224, the method comprising:
a) monitoring the patient's serum triglyceride level after from about 6 weeks to about 52 weeks of being administered the GnRH antagonist during the treatment period;
b) determining that the patient does not exhibit an increase in serum triglyceride level to a level greater than 200 mg/dl; and
c) continuing to administer the GnRH antagonist to the patient for the remainder of the treatment period.

226. The method of any one of claims 1-224, the method comprising:
a) monitoring the patient's serum triglyceride level after from about 6 weeks to about 52 weeks of being administered the GnRH antagonist during the treatment period;
b) determining that the patient does not exhibit an increase in serum triglyceride level from a level less than 300 mg/dl to a level greater than 500 mg/dl; and
c) continuing to administer the GnRH antagonist to the patient for the remainder of the treatment period.

227. A kit comprising a GnRH antagonist and a package insert instructing a user of the kit to administer the GnRH antagonist to a human patient in accordance with the method of any one of claims 1-226.

228. The kit of claim 227, wherein the GnRH antagonist is a compound represented by formula (I) 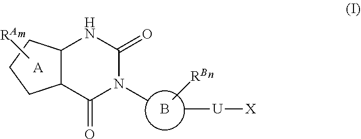
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof.

229. The kit of claim 228, wherein the ring A is a thiophene ring represented by formula (IIa)

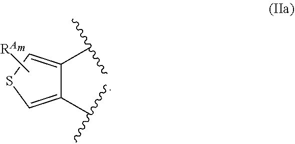

230. The kit of claim 228 or 229, wherein m is 1.

231. The kit of claim 230, wherein the ring A is an optionally substituted thiophene ring represented by formula (IIb)

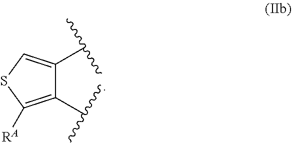

232. The kit of any one of claims 228-231, wherein each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

233. The kit of claim 232, wherein each R^{A} is COOH or pharmaceutically acceptable salt thereof.

234. The kit of any one of claims 228-233, wherein the ring B is an optionally substituted benzene ring, pyridine ring, or thiophene ring.

235. The kit of claim 234, wherein the ring B is represented by a formula selected from the group consisting of:

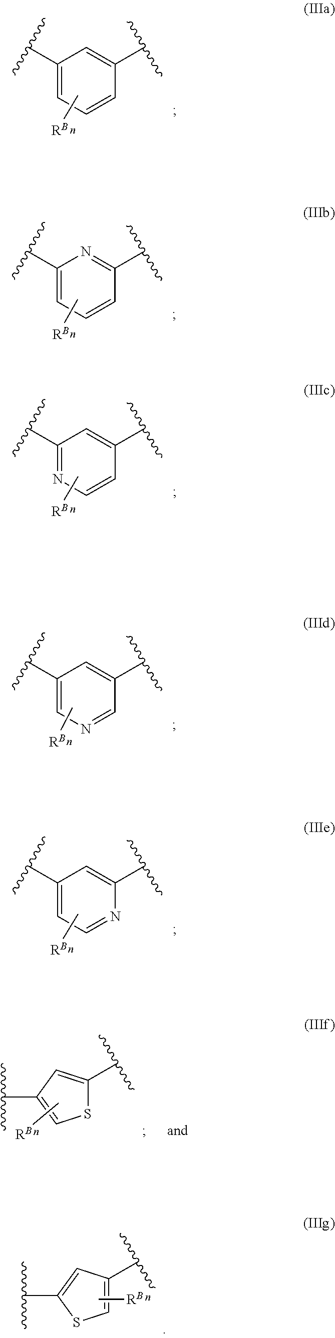

236. The kit of any one of claims 228-235, wherein n is 2.

237. The kit of claim 236, wherein ring B is represented by a formula selected from the group consisting of:

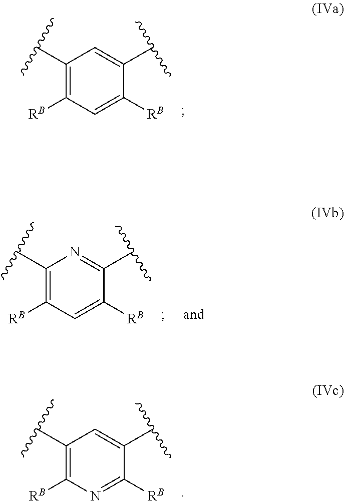

238. The kit of any one of claims 228-237, wherein each R^{B} is independently a halogen atom, an optionally substituted lower alkyl group, or OW⁴, wherein each W⁴ is independently a hydrogen atom or an optionally substituted lower alkyl group.

239. The kit of claim 238, wherein each R^{B} is independently a fluorine atom, chlorine atom, bromine atom, methyl group, or methoxy group.

240. The kit of any one of claims 228-239, wherein U is a single bond.

241. The kit of any one of claims 228-240, wherein X is a group represented by —O-L-Y.

242. The kit of any one of claims 228-241, wherein L is a methylene group.

243. The kit of any one of claims 228-242, wherein Y is an optionally substituted benzene ring represented by formula (V) 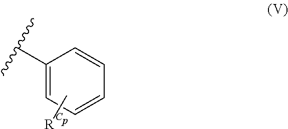
wherein each R^{C} is independently a halogen atom, an optionally substituted lower alkyl group, or OW⁹, wherein each W⁹ is independently a hydrogen atom or an optionally substituted lower alkyl group; and
p is an integer from 0 to 3.

244. The kit of claim 243, wherein Y is a substituted benzene ring represented by formula (Va)

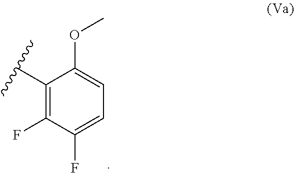 [SMILES: *c1c(OC)ccc(F)c1F]

245. The kit of claim 228, wherein the compound is represented by formula (Ia) 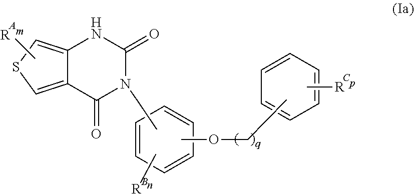
wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
q is an integer from 0 to 3;
each R^{C} is independently a halogen atom, an optionally substituted lower alkyl group, or OW⁹, wherein each W⁹ is independently a hydrogen atom or an optionally substituted lower alkyl group; and
p is an integer from 0 to 3;
or a pharmaceutically acceptable salt thereof.

246. The kit of claim 245, wherein the compound is represented by formula (Ib)

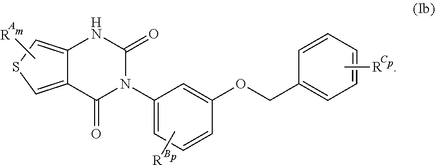

247. The kit of claim 246, wherein the compound is represented by formula (Ic) 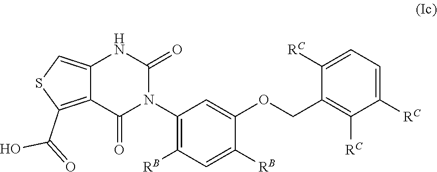
or a pharmaceutically acceptable salt thereof.

248. The kit of any one of claims 228-247, wherein the compound is represented by formula (VI) 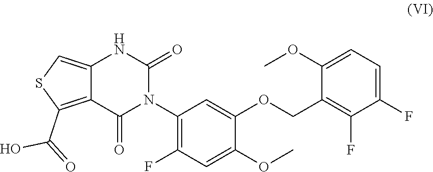 [SMILES: COc1cc(F)c(-n2c(=O)[nH]c3csc(C(=O)O)c3c2=O)cc1OCc1c(OC)ccc(F)c1F]
or a pharmaceutically acceptable salt thereof.

249. The kit of claim 248, wherein the compound is the choline salt of the compound represented by formula (VI).

250. The kit of claim 227, wherein the GnRH antagonist is elagolix, relugolix, opigolix (ASP1707), BAY-784, or SK-2706.

251. A method of treating an estrogen-dependent disease or alleviating one or more symptoms of an estrogen-dependent disease in a human patient in need thereof, the method comprising:
a) periodically administering to the patient a therapeutically effective amount of a GnRH antagonist during a treatment period,
b) determining that the patient does not exhibit a significant increase in total cholesterol level relative to a measurement of the patient's total cholesterol level obtained prior to the treatment period, optionally wherein the determining occurs from about 6 weeks to about 52 weeks after the onset of the treatment period, and
c) continuing to periodically administer the GnRH antagonist to the patient for the remainder of the treatment period.

252. A method of treating an estrogen-dependent disease or alleviating one or more symptoms of an estrogen-dependent disease in a human patient in need thereof, the method comprising:
a) periodically administering to the patient a therapeutically effective amount of a GnRH antagonist during a treatment period,
b) determining that the patient does not exhibit a significant increase in low-density lipoprotein-cholesterol level relative to a measurement of the patient's low-density lipoprotein-cholesterol level obtained prior to the treatment period, optionally wherein the determining occurs from about 6 weeks to about 52 weeks after the onset of the treatment period, and
c) continuing to periodically administer the GnRH antagonist to the patient for the remainder of the treatment period.

253. A method of treating an estrogen-dependent disease or alleviating one or more symptoms of an estrogen-dependent disease in a human patient in need thereof, the method comprising:
a) periodically administering to the patient a therapeutically effective amount of a GnRH antagonist during a treatment period,
b) determining that the patient does not exhibit a significant increase in their ratio of ratio of low-density lipoprotein-cholesterol to high-density lipoprotein-cholesterol relative to a measurement of the patient's ratio of low-density lipoprotein-cholesterol to high-density lipoprotein-cholesterol obtained prior to the treatment period, optionally wherein the determining occurs from about 6 weeks to about 52 weeks after the onset of the treatment period, and
c) continuing to periodically administer the GnRH antagonist to the patient for the remainder of the treatment period.

254. A method of treating an estrogen-dependent disease or alleviating one or more symptoms of an estrogen-dependent disease in a human patient in need thereof, the method comprising:
a) periodically administering to the patient a therapeutically effective amount of a GnRH antagonist during a treatment period,
b) determining that the patient does not exhibit an increase in low-density lipoprotein-cholesterol level to a level greater than 160 mg/dl, optionally wherein the determining occurs from about 6 weeks to about 52 weeks after the onset of the treatment period, and
c) continuing to periodically administer the GnRH antagonist to the patient for the remainder of the treatment period.

255. A method of treating an estrogen-dependent disease or alleviating one or more symptoms of an estrogen-dependent disease in a human patient in need thereof, the method comprising:
a) periodically administering to the patient a therapeutically effective amount of a GnRH antagonist during a treatment period,
b) determining that the patient does not exhibit an increase in low-density lipoprotein-cholesterol level from a level less than 160 mg/dl (e.g., from a level less than 130 mg/dl) to a level greater than 190 mg/dl, optionally wherein the determining occurs from about 6 weeks to about 52 weeks after the onset of the treatment period, and
c) continuing to periodically administer the GnRH antagonist to the patient for the remainder of the treatment period.

256. A method of treating an estrogen-dependent disease or alleviating one or more symptoms of an estrogen-dependent disease in a human patient in need thereof, the method comprising:
a) periodically administering to the patient a therapeutically effective amount of a GnRH antagonist during a treatment period,
b) determining that the patient does not exhibit an increase in serum triglyceride level of greater than 30% relative to a measurement of the patient's serum triglyceride level obtained prior to the treatment period, optionally wherein the determining occurs from about 6 weeks to about 52 weeks after the onset of the treatment period, and
c) continuing to periodically administer the GnRH antagonist to the patient for the remainder of the treatment period.

257. A method of treating an estrogen-dependent disease or alleviating one or more symptoms of an estrogen-dependent disease in a human patient in need thereof, the method comprising:
a) periodically administering to the patient a therapeutically effective amount of a GnRH antagonist during a treatment period,
b) determining that the patient does not exhibit an increase in serum triglyceride level to a level greater than 200 mg/dl, optionally wherein the determining occurs from about 6 weeks to about 52 weeks after the onset of the treatment period, and
c) continuing to periodically administer the GnRH antagonist to the patient for the remainder of the treatment period.

258. A method of treating an estrogen-dependent disease in a human patient in need thereof, the method comprising:
a) periodically administering to the patient a therapeutically effective amount of a GnRH antagonist during a treatment period,
b) determining that the patient does not exhibit an increase in serum triglyceride level from a level less than 300 mg/dl to a level greater than 500 mg/dl, optionally wherein the determining occurs from about 6 weeks to about 52 weeks after the onset of the treatment period, and
c) continuing to periodically administer the GnRH antagonist to the patient for the remainder of the treatment period.

259. The method of any one of claims 251-258, wherein the estrogen-dependent disease is uterine fibroids, endometriosis, adenomyosis, or rectovaginal endometriosis.

260. The method of any one of claims 251-259, wherein the GnRH antagonist is a compound represented by formula (I) 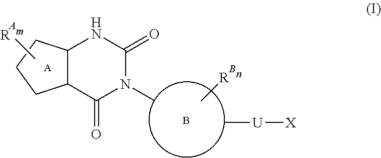
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁵, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof.

261. The method of claim 260, wherein the ring A is a thiophene ring represented by formula (IIa)

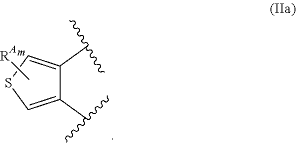

262. The method of claim 260 or 261, wherein m is 1.

263. The method of claim 262, wherein the ring A is an optionally substituted thiophene ring represented by formula (IIb)

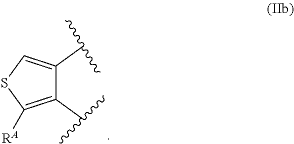

264. The method of any one of claims 260-263, wherein each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

265. The method of claim 264, wherein each R^{A} is COOH or pharmaceutically acceptable salt thereof.

266. The method of any one of claims 260-265, wherein the ring B is an optionally substituted benzene ring, pyridine ring, or thiophene ring.

267. The method of claim 266, wherein the ring B is represented by a formula selected from the group consisting of:

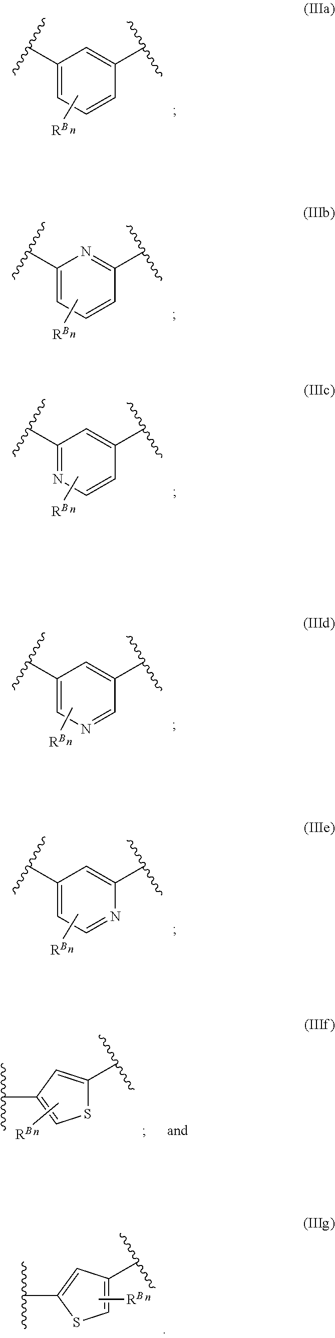

268. The method of any one of claims 260-267, wherein n is 2.

269. The method of claim 268, wherein the ring B is represented by a formula selected from the group consisting of:

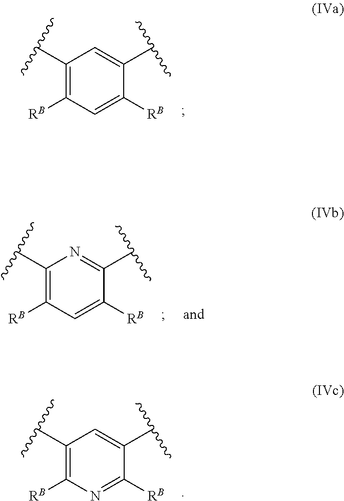

270. The method of any one of claims 260-269, wherein each R^{B} is independently a halogen atom, an optionally substituted lower alkyl group, or OW⁴, wherein each W⁴ is independently a hydrogen atom or an optionally substituted lower alkyl group.

271. The method of claim 270, wherein each R^{B} is independently a fluorine atom, chlorine atom, bromine atom, methyl group, or methoxy group.

272. The method of any one of claims 260-271, wherein U is a single bond.

273. The method of any one of claims 260-272, wherein X is a group represented by —O-L-Y.

274. The method of any one of claims 260-273, wherein L is a methylene group.

275. The method of any one of claims 260-274, wherein Y is an optionally substituted benzene ring represented by formula (V) 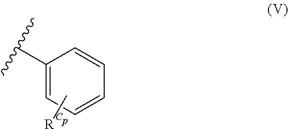
wherein each R^{C} is independently a halogen atom, an optionally substituted lower alkyl group, or OW⁹, wherein each W⁹ is independently a hydrogen atom or an optionally substituted lower alkyl group; and
p is an integer from 0 to 3.

276. The method of claim 275, wherein Y is a substituted benzene ring represented by formula (Va)

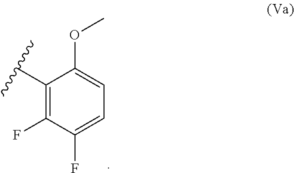 [SMILES: *c1c(OC)ccc(F)c1F]

277. The method of claim 260, wherein the compound is represented by formula (Ia) 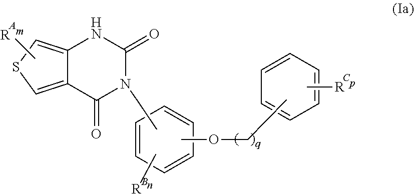
wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
q is an integer from 0 to 3;
each R^{C} is independently a halogen atom, an optionally substituted lower alkyl group, or OW⁹, wherein each W⁹ is independently a hydrogen atom or an optionally substituted lower alkyl group; and
p is an integer from 0 to 3;
or a pharmaceutically acceptable salt thereof.

278. The method of claim 277, wherein the compound is represented by formula (Ib)

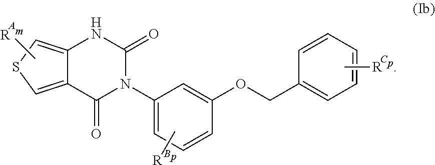

279. The method of claim 278, wherein the compound is represented by formula (Ic) 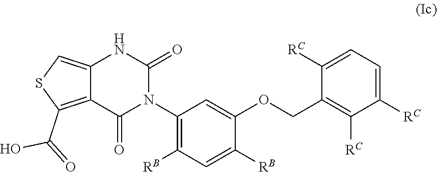
or a pharmaceutically acceptable salt thereof.

280. The method of claim any one of claims 260-279, wherein the compound is represented by formula (VI) 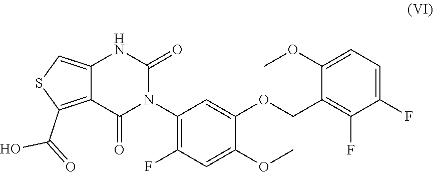 [SMILES: COc1cc(F)c(-n2c(=O)[nH]c3csc(C(=O)O)c3c2=O)cc1OCc1c(OC)ccc(F)c1F]
or a pharmaceutically acceptable salt thereof.

281. The method of claim 280, wherein the compound is the choline salt of the compound represented by formula (VI).

282. The method of claim 281, wherein the compound is in a crystalline state.

283. The method of claim 282, wherein the compound exhibits characteristic X-ray powder diffraction (XRPD) peaks at about 7.1° 2θ, about 11.5° 2θ, about 19.4° 2θ, about 21.5° 2θ, about 22.0° 20, about 22.6° 2θ, about 23.5° 2θ, and about 26.2° 2θ.

284. The method of claim 282 or 283, wherein the compound exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm.

285. The method of any one of claims 282-284, wherein the compound exhibits ¹⁹F solid-state NMR peaks centered at about −151.8 ppm, −145.2 ppm, and −131.6 ppm.

286. The method of any one of claims 260-285, wherein the compound is orally administered to the patient.

287. The method of any one of claims 260-286, wherein the compound is administered to the patient one or more times per day, week, or month during the treatment period.

288. The method of claim 287, wherein the compound is administered to the patient one or more times daily during the treatment period.

289. The method of claim 288, wherein the compound is administered to the patient once daily during the treatment period.

290. The method of any one of claims 287-289, wherein the compound is administered to the patient in an amount of from about 25 mg per day to about 400 mg per day during the treatment period.

291. The method of claim 290, wherein the compound is administered to the patient in an amount of from about 35 mg to about 65 mg per day during the treatment period.

292. The method of claim 291, wherein the compound is administered to the patient in an amount of about 50 mg per day during the treatment period.

293. The method of claim 290, wherein the compound is administered to the patient in an amount of from about 60 mg per day to about 90 mg per day during the treatment period.

294. The method of claim 293, wherein the compound is administered to the patient in an amount of about 75 mg per day during the treatment period.

295. The method of claim 290, wherein the compound is administered to the patient in an amount of from about 85 mg to about 115 mg per day during the treatment period.

296. The method of claim 295, wherein the compound is administered to the patient in an amount of about 100 mg per day during the treatment period.

297. The method of claim 290, wherein the compound is administered to the patient in an amount of from about 185 mg to about 215 mg per day during the treatment period.

298. The method of claim 297, wherein the compound is administered to the patient in an amount of about 200 mg per day during the treatment period.

299. The method of any one of claims 251-298, wherein the determining occurs from about 6 weeks to about 52 weeks after the onset of the treatment period.

300. The method of any one of claims 251-298, wherein the determining occurs from about 6 weeks to about 48 weeks after the onset of the treatment period.

301. The method of any one of claims 251-298, wherein the determining occurs from about 6 weeks to about 44 weeks after the onset of the treatment period.

302. The method of any one of claims 251-298, wherein the determining occurs from about 6 weeks to about 42 weeks after the onset of the treatment period.

303. The method of any one of claims 251-298, wherein the determining occurs from about 6 weeks to about 38 weeks after the onset of the treatment period.

304. The method of any one of claims 251-298, wherein the determining occurs from about 6 weeks to about 36 weeks after the onset of the treatment period.

305. The method of any one of claims 251-298, wherein the determining occurs from about 6 weeks to about 32 weeks after the onset of the treatment period.

306. The method of any one of claims 251-298, wherein the determining occurs from about 6 weeks to about 28 weeks after the onset of the treatment period.

307. The method of any one of claims 251-298, wherein the determining occurs from about 6 weeks to about 24 weeks after the onset of the treatment period.

308. The method of any one of claims 251-298, wherein the determining occurs from about 6 weeks to about 18 weeks after the onset of the treatment period.

309. The method of any one of claims 251-298, wherein the determining occurs from about 6 weeks to about 16 weeks after the onset of the treatment period.

310. The method of any one of claims 251-298, wherein the determining occurs from about 6 weeks to about 12 weeks after the onset of the treatment period.

311. The method of any one of claims 251-298, wherein the determining occurs from about 12 weeks to about 36 weeks after the onset of the treatment period.

312. The method of any one of claims 251-298, wherein the determining occurs from about 16 weeks to about 32 weeks after the onset of the treatment period.

313. The method of any one of claims 251-298, wherein the determining occurs from about 20 weeks to about 28 weeks after the onset of the treatment period.

314. The method of any one of claims 251-298, wherein the determining occurs from about 21 weeks to about 27 weeks after the onset of the treatment period.

315. The method of any one of claims 251-298, wherein the determining occurs from about 22 weeks to about 26 weeks after the onset of the treatment period.

316. The method of any one of claims 251-298, wherein the determining occurs from about 23 weeks to about 25 weeks after the onset of the treatment period.

317. The method of any one of claims 251-298, wherein the determining occurs about 6 weeks after the onset of the treatment period.

318. The method of any one of claims 251-298, wherein the determining occurs about 8 weeks after the onset of the treatment period.

319. The method of any one of claims 251-298, wherein the determining occurs about 10 weeks after the onset of the treatment period.

320. The method of any one of claims 251-298, wherein the determining occurs about 12 weeks after the onset of the treatment period.

321. The method of any one of claims 251-298, wherein the determining occurs about 14 weeks after the onset of the treatment period.

322. The method of any one of claims 251-298, wherein the determining occurs about 16 weeks after the onset of the treatment period.

323. The method of any one of claims 251-298, wherein the determining occurs about 18 weeks after the onset of the treatment period.

324. The method of any one of claims 251-298, wherein the determining occurs about 20 weeks after the onset of the treatment period.

325. The method of any one of claims 251-298, wherein the determining occurs about 22 weeks after the onset of the treatment period.

326. The method of any one of claims 251-298, wherein the determining occurs about 24 weeks after the onset of the treatment period.

327. The method of any one of claims 251-298, wherein the determining occurs about 26 weeks after the onset of the treatment period.

328. The method of any one of claims 251-298, wherein the determining occurs about 28 weeks after the onset of the treatment period.

329. The method of any one of claims 251-298, wherein the determining occurs about 30 weeks after the onset of the treatment period.

330. The method of any one of claims 251-298, wherein the determining occurs about 32 weeks after the onset of the treatment period.

331. The method of any one of claims 251-298, wherein the determining occurs about 34 weeks after the onset of the treatment period.

332. The method of any one of claims 251-298, wherein the determining occurs about 36 weeks after the onset of the treatment period.

333. The method of any one of claims 251-298, wherein the determining occurs about 38 weeks after the onset of the treatment period.

334. The method of any one of claims 251-298, wherein the determining occurs about 40 weeks after the onset of the treatment period.

335. The method of any one of claims 251-298, wherein the determining occurs about 42 weeks after the onset of the treatment period.

336. The method of any one of claims 251-298, wherein the determining occurs about 44 weeks after the onset of the treatment period.

337. The method of any one of claims 251-298, wherein the determining occurs about 46 weeks after the onset of the treatment period.

338. The method of any one of claims 251-298, wherein the determining occurs about 48 weeks after the onset of the treatment period.

339. The method of any one of claims 251-298, wherein the determining occurs about 50 weeks after the onset of the treatment period.

340. The method of any one of claims 251-298, wherein the determining occurs about 52 weeks after the onset of the treatment period.
